(19) United States
(12) Patent Application Publication
(10) Pub. No.: US 2025/0034197 A1
(43) Pub. Date: Jan. 30, 2025

(54) NUCLEOSIDE-DIPHOSPHATE-HEPTOSE COMPOUNDS FOR TREATING CONDITIONS ASSOCIATED WITH ALPK1 ACTIVITY

(71) Applicant: PYROTECH (BEIJING) BIOTECHNOLOGY CO., LTD., Beijing (CN)

(72) Inventors: Nana Du, Beijing (CN); Counde O'Yang, Sunnyvale, CA (US); Hexiang Wang, Beijing (CN); Zhihong Li, Millbrae, CA (US)

(21) Appl. No.: 18/696,715

(22) PCT Filed: Sep. 29, 2022
(86) PCT No.: PCT/CN2022/122516

§ 371 Date: Mar. 28, 2024

(30) Foreign Application Priority Data
Sep. 30, 2021 (WO) PCT/CN2021/122250
Dec. 16, 2021 (WO) PCT/CN2021/138786
Jun. 17, 2022 (WO) PCT/CN2022/099495
Sep. 15, 2022 (WO) PCT/CN2022/118981

(51) Int. Cl. C07H 19/207; A61K 31/7072; A61K 31/7076; A61K 39/00; A61K 39/39; C07H 19/10

(52) U.S. Cl. CPC C07H 19/207; A61K 31/7072; A61K 31/7076; A61K 39/39; C07H 19/10; A61K 2039/55511

(57) ABSTRACT

The invention concerns compounds of Formula (X) which modulate, especially agonize alpha kinase 1 (ALPK1) and are useful for treating a condition, disease or disorder in which a decrease or increase in ALPK1 activity contributes to the pathology or symptoms or progression of the condition, disease or disorder such as cancer, immune or inflammatory related diseases.

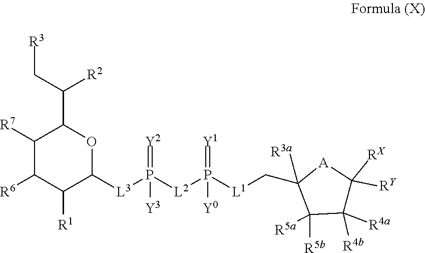

### CROSS REFERENCE TO RELATED APPLICATIONS

The present application is a national stage entry under 35 USC § 371 of International Application No. PCT/CN2022/122516 filed on Sep. 29, 2022, which claims priority to the PCT Application No. PCT/CN2021/122250 filed on Sep. 30, 2021, the PCT Application No. PCT/CN2021/138786 filed on Dec. 16, 2021, the PCT Application No. PCT/CN2022/099495 filed on Jun. 17, 2022, and the PCT Application No. PCT/CN2022/118981 filed on Sep. 15, 2022, the contents of which are hereby incorporated by reference in their entirety.

### TECHNICAL FIELD

This disclosure features chemical entities (e.g., a compound that modulates (e.g., agonizes) alpha kinase 1 (ALPK1), or a pharmaceutically acceptable salt, and/or hydrate, and/or cocrystal, and/or tautomer, and/or stereoisomer, and/or stable isotope, and/or prodrug, and/or drug combination of the compound) that are useful, e.g., for treating a condition, disease or disorder in which a decrease or increase in ALPK1 activity (e.g., a decrease, e.g., a condition, disease or disorder associated with repressed or impaired ALPK1 signaling) contributes to the pathology and/or symptoms and/or progression of the condition, disease or disorder (e.g., cancer; or e.g., immune and/or inflammatory related diseases (e.g., IBD)) in a subject (e.g., a human). This disclosure also features compositions as well as other methods of using and making the same.

### BACKGROUND

Alpha kinase 1 (ALPK1) has previously been identified as having a critical role in the immune system with respect to responding to bacterial infection. ALPK1 is a host cytosolic protein serving as the receptor for ADP-Heptose, a natural bacterial product, during biosynthesis of LPS. When ADP-Heptose binds ALPK1, the kinase activity of ALPK1 is activated, inducing TRAF-interacting protein with a forkhead-associated domain (TIFA) phosphorylation. Ultimately, the NF-κB pathway is activated and enhances cytokine transcription, leading to the activation of host immune system. (Gaudet et al., 2015; Milivojevic et al., 2017; Zimmermann et al., 2017; Zhou et al., 2018; Pfannkuch et al., 2019)

A variety of studies and clinical trials have demonstrated that boosting immune responses benefits patients suffering from diseases, such as cancer and immune and/or inflammatory diseases. Although several immune activating agents been approved for clinical use, new treatment options are still needed.

WO 2019/238024, US 2019/0367553, WO 2020/216327, and WO 2019/080898 (each incorporated herein by reference in its entirety) disclose modulators of ALPK1.

### SUMMAR^{Y}

This disclosure features chemical entities (e.g., a compound that modulates (e.g., agonizes) alpha kinase 1 (ALPK1), or a pharmaceutically acceptable salt, and/or hydrate, and/or cocrystal, and/or tautomer, and/or stereoisomer, and/or stable isotope, and/or prodrug, and/or drug combination of the compound) that are useful, e.g., for treating a condition, disease or disorder in which a decrease or increase in ALPK1 activity (e.g., a decrease, e.g., a condition, disease or disorder associated with repressed or impaired ALPK1 signaling) contributes to the pathology and/or symptoms and/or progression of the condition, disease or disorder (e.g., cancer; or e.g., immune and/or inflammatory related diseases (e.g., IBD)) in a subject (e.g., a human). This disclosure also features compositions as well as other methods of using and making the same.

In one aspect, this disclosure features compounds of Formula (X):

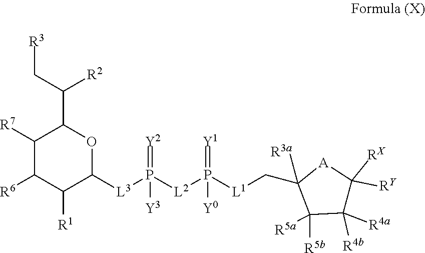

or a pharmaceutically acceptable salt thereof, or a stereoisomer, a stable isotope, a prodrug, or a tautomer thereof, wherein:

R^{X}, R^{Y}, R¹, R², R³, R^{3a}, R^{4a}, R^{4b}, R^{5a}, R^{5b}, R⁶, R⁷, A, L¹, L², L³, Y⁰, Y¹, Y², and Y³ can be as defined anywhere herein.

In one aspect, pharmaceutical compositions are featured that include a chemical entity described herein (e.g., a compound described generically or specifically herein or a pharmaceutically acceptable salt thereof or compositions containing the same) and one or more pharmaceutically acceptable excipients.

In one aspect, methods for modulating (e.g., agonizing) ALPK1 activity are featured that include contacting ALPK1 with a chemical entity described herein (e.g., a compound described generically or specifically herein or a pharmaceutically acceptable salt thereof or compositions containing the same). Methods include in vitro methods, e.g., contacting a sample that includes one or more cells comprising ALPK (e.g., innate immune cells, e.g., mast cells, macrophages, dendritic cells (DCs), and natural killer cells) with the chemical entity. The contacting can, in some cases, induce an immune response sufficient to kill at least one of the one or more cancer cells. Methods can also include in vivo methods; e.g., administering the chemical entity to a subject (e.g., a human) having a disease in which repressed or impaired ALPK signaling contributes to the pathology and/or symptoms and/or progression of the disease (e.g., cancer; e.g., a refractory cancer).

In another aspect, methods of treating immune and/or inflammatory related disease are featured that include administering to a subject in need of such treatment an effective amount of a chemical entity described herein (e.g., a compound described generically or specifically herein or a pharmaceutically acceptable salt thereof or compositions containing the same). In some embodiments, immune and/or inflammatory related disease is inflammatory bowel disease. In some embodiments, the immune and/or inflammatory related disease is ulcerative colitis. In some embodiments, the immune and/or inflammatory related disease is Crohn's disease.

In a further aspect, methods of treating cancer are featured that include administering to a subject in need of such treatment an effective amount of a chemical entity described herein (e.g., a compound described generically or specifically herein or a pharmaceutically acceptable salt thereof or compositions containing the same). In some embodiments, the cancer is selected from the group consisting of: brain cancer, skin cancer, bladder cancer, ovarian cancer, breast cancer, gastric cancer, pancreatic cancer, hepatocellular cancer, prostate cancer, colorectal cancer, blood cancer, lung cancer, and bone cancer. In certain embodiments, the cancer is selected from the group consisting of: small cell lung cancer, non-small cell lung cancer, colorectal cancer, melanoma, renal cell carcinoma, head and neck cancer, Hodgkin's lymphoma, and bladder cancer.

In another aspect, methods of enhancing the efficacy of a vaccine are featured that include administering to a subject in need of such treatment an effective amount of a chemical entity described herein (e.g., a compound described generically or specifically herein or a pharmaceutically acceptable salt thereof or compositions containing the same). In some embodiments, the vaccine is a cancer vaccine. In some embodiments, the vaccine is a bacterial vaccine. In some embodiments, the vaccine is a viral vaccine. In some embodiments, the vaccine is a parasite vaccine. In some embodiments, the chemical entity described herein is an adjuvant.

In a further aspect, methods of enhancing innate immunity are featured that include administering to a subject in need of such treatment an effective amount of a chemical entity described herein (e.g., a compound described generically or specifically herein or a pharmaceutically acceptable salt thereof or compositions containing the same).

In another aspect, methods of inducing an immune response (e.g., an innate immune response) in a subject in need thereof are featured that include administering to the subject an effective amount of a chemical entity described herein (e.g., a compound described generically or specifically herein or a pharmaceutically acceptable salt thereof or compositions containing the same).

In a further aspect, methods of promoting systemic immune responses in a subject in need thereof are featured that include administering to the subject an effective amount of a chemical entity described herein (e.g., a compound described generically or specifically herein or a pharmaceutically acceptable salt thereof or compositions containing the same).

In another aspect, methods of inducing cytokine production and/or NF-κB pathway activation in a subject in need thereof are featured that include administering to the subject an effective amount of a chemical entity described herein (e.g., a compound described generically or specifically herein or a pharmaceutically acceptable salt thereof or compositions containing the same).

In a further aspect, methods of treatment of a disease in which repressed or impaired ALPK1 signaling contributes to the pathology and/or symptoms and/or progression of the disease are featured that include administering to a subject in need of such treatment an effective amount of a chemical entity described herein (e.g., a compound described generically or specifically herein or a pharmaceutically acceptable salt thereof or compositions containing the same).

In another aspect, methods of treatment are featured that include administering to a subject having a disease in which repressed or impaired ALPK1 signaling contributes to the pathology and/or symptoms and/or progression of the disease an effective amount of a chemical entity described herein (e.g., a compound described generically or specifically herein or a pharmaceutically acceptable salt thereof or compositions containing the same).

In a further aspect, methods of treatment that include administering to a subject a chemical entity described herein (e.g., a compound described generically or specifically herein or a pharmaceutically acceptable salt thereof or compositions containing the same), wherein the chemical entity is administered in an amount effective to treat a disease in which repressed or impaired ALPK1 signaling contributes to the pathology and/or symptoms and/or progression of the disease, thereby treating the disease.

Embodiments can include one or more of the following features.

The chemical entities disclosed herein can be administered in combination with one or more additional therapeutic agents. For example, the chemical entities disclosed herein can be administered with one or more immunotherapeutic agents. The one or more immunotherapeutic agents can comprise a small molecule, an antibody, and/or a cytokine. In some embodiments, the immunotherapeutic agent is an inhibitor/antagonist of an inhibitory (including co-inhibitory) immune checkpoint. In some embodiments, the immunotherapeutic agent is an antagonist of an inhibitory/co-inhibitory immune checkpoint. In some embodiments, the immunotherapeutic agent is an agonist of a stimulatory/co-stimulatory receptor.

Non-limiting examples of immune checkpoints include PD-1 and PD-L¹. In some embodiments, the immunotherapeutic is a therapeutic monoclonal antibody. In some embodiments, the antibody is selected from the group consisting nivolumab, pembrolizumab, pidilizumab, cemiplimab, camrelizumab, tislelizumab, BMS-936559, atezolizumab, durvalumab, and avelumab. In some embodiments, the antibody is nivolumab or pembrolizumab. In some embodiments, the immune checkpoint is CTLA-4. In some embodiments, the antibody is ipilimumab. In some embodiments, the immune checkpoint is TIGIT. In some embodiments, the antibody is an inhibitory antibody of TIGIT.

In some embodiments, the immunotherapeutic agent is an activator/agonist of a stimulatory (including co-stimulatory) signal on immune cells, (e.g., T cells). The stimulatory/co-stimulatory proteins for the combination therapy of the invention are noted herein. In some embodiments, the stimulatory proteins include, but are not limited to, 4-1BB or OX40. In some embodiments, the agonist is a therapeutic monoclonal antibody specific for the activation 4-1BB or OX40.

The subject can have cancer, e.g., the subject has undergone and/or is undergoing and/or will undergo one or more cancer therapies.

Non-limiting examples of cancer include melanoma, cervical cancer, breast cancer, ovarian cancer, hepatocellular cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma. In certain embodiments, the cancer can be a refractory cancer.

The chemical entity can be administered via administering comprises intramuscular, intraperitoneal, or intravenous administration.

The chemical entity can be administered intratumorally.

The methods can further include identifying the subject.

Other embodiments include those described in the Detailed Description and/or in the claims.

### Additional Definitions

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art, for example, the nomenclature can be generated by using the software ChemDraw. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

The term “comprising”, “including”, “having”, or “containing” means “including but not limited to” as well as “consisting of′, e.g. a composition “comprising” X may consist exclusively of X or may include something additional e.g. X+Y. Additionally, whenever “comprising” or another open-ended term is used in an embodiment, it is to be understood that the same embodiment can be more narrowly claimed using the intermediate term “consisting essentially of′ or the closed term “consisting of”. As used herein, the articles “a” and “an” refer to one or to more than one (e.g., to at least one) of the grammatical object of the article. The term “or” is used herein to mean, and is used interchangeably with, the term “and/or”, unless context clearly indicates otherwise.

As used herein, the term “ALPK1 agonists” refers to any compound that can activate the kinase activity of ALPK1, and consequently increase and/or stimulate an immune response. ALPK1 kinase activity is measured by the TIFA (TRAF-interacting protein with fork head-associated domain) phosphorylation assay as described herein. Non-limiting examples include: UDPS-Heptose, ADPS-Heptose, or CDPS-Heptose. In some embodiments, the compound is selected from the group consisting of compounds of Formula (X), Formula (I-h), Formula (I-h-1), Formula (I-h-2), Formula (I-h-3), Formula (I-h-4), Formula (I-h-5), Formula (I-k), Formula (I-k-1), Formula (I-k-2), Formula (I-k-3), Formula (I-k-4), or Formula (I-k-5) (hereinafter referred to as “Formulae disclosed herein”).

As used herein, the “immunotherapy agent” or “immune modulator” refers to a small molecule drug, antibody, or other biologic molecules. In some embodiments, the modulator is used to inhibit an inhibitory immune receptor signal on T-cells, and/or other immune cells, such as dendritic cells. In some embodiments, the modulator is used to enhance and/or stimulate a co-stimulatory immune receptor signal on T-cells, and/or other immune cells, such as dendritic cells. In some embodiments, the biologic immune modulator includes, but is not limited to, cancer vaccines, antibodies, and cytokines. In some embodiments, the antibody is a monoclonal antibody. In another aspect, the monoclonal antibody is humanized.

The terms “treat,” “treating,” and “treatment,” in the context of treating a disease, disorder, or condition are meant to include alleviating or abrogating a disorder, disease, or condition, or one or more of the symptoms associated with the disorder, disease, or condition; or slowing the progression, spread or worsening of a disease, disorder or condition or of one or more symptoms thereof. Often, the beneficial effects that a subject derives from a therapeutic agent do not result in a complete cure of the disease, disorder or condition. In some embodiments, the terms “treat,” “treating,” and “treatment,” include virologically curing a viral disorder, disease, or condition; reducing viral shedding; decreasing viral RNA load (e.g., a measured by PCR); reducing the length of stay in a hospital; reducing the length of stay in an infectious disease unit and/or intensive care unit; or slowing (including stopping) the progression/development of respiratory (or other serious) symptoms.

The “treatment of cancer”, refers to one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

The term “therapeutically effective amount” refers to the amount of a drug or other pharmaceutical agent (e.g., a compound disclosed herein), that will elicit the biological and/or medical response of a tissue, system, animal or human (e.g., subject or patient) that is being sought, for instance, by a researcher or clinician. Furthermore, the term “therapeutically effective amount” means any amount, as compared to a corresponding subject (e.g., patient) who has not received such amount, which is sufficient to decrease the rate of advancement of, prevent development of, or alleviate to some extent, one or more of the symptoms of the condition or disorder being treated. The therapeutically effective amount will vary depending on the compound, the disease and its severity and the age, weight, etc., of the mammal to be treated. In addition, the therapeutically effective amount can be administered in one or more administrations, applications or dosages and is not intended to be limited to a particular formulation or administration route.

As used herein, the term “subject or patient” used interchangeably herein refers to an animal, including, but not limited to, a primate (e.g., human), monkey, cow, pig, sheep, goat, horse, dog, cat, rabbit, rat, or mouse. In some embodiments, the subject is a human to be treated by the methods and compositions of the present disclosure. In some embodiments, the methods described herein further include the step of identifying a subject (e.g., a patient) in need of such treatment (e.g., by way of biopsy, endoscopy, or other conventional method known in the art). In some embodiments, the chemical entities, methods, and compositions described herein can be administered to certain treatment-resistant patient populations (e.g., patients resistant to checkpoint inhibitors; e.g., patients having one or more cold tumors, e.g., tumors lacking T-cells or exhausted T-cells).

The term “vaccine” refers to a biological preparation administered to a human or animal in order to elicit or enhance a specific immune response and/or protection against one or more antigens in that human or animal. In some embodiments, the vaccine is a cancer vaccine against one or more antigens of cancer cell.

The term “adjuvant” refers to a secondary therapeutic substance that is administered together (either sequentially in any order, or concurrently) with a primary therapeutic substance to achieve some kind of complimentary, synergic or otherwise beneficial effect that could not be achieved through use of the primary therapeutic substance alone. An adjuvant can be used together with a vaccine, chemotherapy, or some other therapeutic substance. Adjuvants can enhance the efficacy of the primary therapeutic substance, reduce the toxicity or side effects of the primary therapeutic substance, or provide some kind of protection to the subject that receives the primary therapeutic substance, such as, but not limited to, improved functioning of the immune system.

As used herein, the term “cancer” refers to the physiological condition in subjects that is characterized by unregulated or dysregulated cell growth or death. The term “cancer” includes solid tumors and blood-born tumors, whether malignant or benign.

The term “acceptable” with respect to a formulation, composition or ingredient, as used herein, means having no persistent detrimental effect on the general health of the subject being treated.

“API” refers to an active pharmaceutical ingredient.

The term “excipient” or “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRc Press LLC: Boca Raton, FL, 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described hereinform with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer (such as cis- and trans-isomer), or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses.

In addition, prodrugs are also included within the context of the present disclosure. The term “prodrug” as used herein refers to a compound which is converted in vivo to an active form thereof having a medical effect by, for example, hydrolysis in blood. Pharmaceutically acceptable prodrugs are described in T. Higuchi and V. Stella, Prodrugs as Novel Delivery Systems, A.C.S. Symposium Series, Vol. 14, Edward B. Roche, ed., Bioreversible Carriers in Drug Design, American Pharmaceutical Association and Pergamon Press, 1987, and D. Fleisher, S. Ramon and H. Barbra “Improved oral drug delivery: solubility limitations overcome by the use of prodrugs”, Advanced Drug Delivery Reviews (1996) 19 (2) 115-130, each is incorporated herein by reference.

A prodrug is any covalently bonded carrier which, when administered to a patient, releases the compound of formula (I) in vivo. Prodrugs are typically prepared by modifying functional groups in such a way that the modifications can be cleaved by routine manipulation or in vivo to yield the parent compound. Prodrugs include, for example, compounds disclosed herein wherein a hydroxy, amine or sulfhydryl group is bonded to any group which, when administered to a patient, can be cleaved to form a hydroxy, amine or sulfhydryl group. Thus, representative examples of prodrugs include, but are not limited to, acetate, formate and benzoate derivatives of alcohol, mercapto and amine functional groups of the compounds of formula (I). Further, in the case of a carboxylic acid (—COOH), an ester such as a methyl ester, an ethyl ester or the like can be used. The ester itself may be active and/or may hydrolyze under conditions in human bodies. Suitable pharmaceutically acceptable hydrolysable in vivo ester groups include those groups which readily decompose in the human body to release the parent acid or a salt thereof.

Also disclosed herein are all suitable isotopical derivertives of the compounds disclosed herein. An isotope derivative of a compound disclosed herein is defined as wherein at least one atom is replaced by an atom having the same atomic number but differing in atomic mass from the atomic mass typically found in nature. Examples of isotopes that can be listed as compounds disclosed herein include hydrogen, carbon, nitrogen, oxygen, fluorine, and chlorine isotopes, such as ²H, ³H, ¹³C, ¹⁴C, ¹⁵N, ¹⁷O, ¹⁸O, ¹⁸F, ³¹P, ³²P, ³⁵S and ³⁶Cl, respectively. Certain isotopical derivertives of the compounds disclosed herein, such as the radioisotopes of 3H and ¹⁴C, are also among them and are useful in the tissue distribution experiments of drugs and substrates. Tritium, i.e., ³H, and carbon-14, i.e., ¹⁴C, are easier to prepare and detect and are the first choice for isotopes. In addition, substitution with isotopes such as deuterium, i.e., ²H, has advantages in some therapies due to its good metabolic stability, for example, increased half-life in vivo or reduced dosage, and thus priority may be given in some cases. Isotopical derivertives of the compounds disclosed herein can be prepared by conventional procedures, for example by descriptive methods or by the preparations described in the Examples below, using appropriate isotopic derivatives of the appropriate reagents. The term “stable isotope” refers to those exist stably in nature.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: rectal, oral, intravenous, aerosol, parenteral, ophthalmic, pulmonary, and topical administration.

The term “halo” refers to fluoro (F), chloro (Cl), bromo (Br), or iodo (I).

The term “alkyl” refers to a saturated acyclic hydrocarbon radical that may be a straight chain or branched chain, containing the indicated number of carbon atoms. For example, C1-10 indicates that the group may have from 1 to 10 (inclusive) carbon atoms in it. Alkyl groups can either be unsubstituted or substituted with one or more substituents. Non-limiting examples include methyl, ethyl, iso-propyl, tert-butyl, n-hexyl. The term “saturated” as used in this context means only single bonds present between constituent carbon atoms and other available valences occupied by hydrogen and/or other substituents as defined herein.

The term “haloalkyl” refers to an alkyl, in which one or more hydrogen atoms is/are replaced with an independently selected halo.

The term “alkoxy” refers to an -O-alkyl radical (e.g., —OCH₃).

The term “alkylene” refers to a divalent alkyl (e.g., —CH₂—).

The term “alkenyl” refers to an acyclic hydrocarbon chain that may be a straight chain or branched chain having one or more carbon-carbon double bonds. The alkenyl moiety contains the indicated number of carbon atoms. For example, C₂₋₆ indicates that the group may have from 2 to 6 (inclusive) carbon atoms in it. Alkenyl groups can either be unsubstituted or substituted with one or more substituents. The term “alkenyl” also includes an acyclic hydrocarbon chain with cumulated diene, i.e., two adjacent carbon-carbon double bonds are present and one carbon atom is common to two carbon-carbon double bonds, for example,

The term “alkynyl” refers to an acyclic hydrocarbon chain that may be a straight chain or branched chain having one or more carbon-carbon triple bonds. The alkynyl moiety contains the indicated number of carbon atoms. For example, C₂₋₆ indicates that the group may have from 2 to 6 (inclusive) carbon atoms in it. Alkynyl groups can either be unsubstituted or substituted with one or more substituents.

The term “aryl” refers to a 6-20 carbon mono-, bi-, tri- or polycyclic group wherein at least one ring in the system is aromatic (e.g., 6-carbon monocyclic, 10-carbon bicyclic, or 14-carbon tricyclic aromatic ring system); and wherein 0, 1, 2, 3, or 4 atoms of each ring may be substituted by a substituent. Examples of aryl groups include phenyl, naphthyl, tetrahydronaphthyl, dihydro-1H-indenyl and the like.

The term “cycloalkyl” as used herein refers to cyclic saturated hydrocarbon groups having, e.g., 3 to 20 ring carbons, preferably 3 to 16 ring carbons, and more preferably 3 to 12 ring carbons or 3-10 ring carbons or 3-6 ring carbons, wherein the cycloalkyl group may be optionally substituted. Examples of cycloalkyl groups include, without limitation, cyclopropyl, cyclobutyl, cyclopentyl, cyclohexyl, cycloheptyl, and cyclooctyl. Cycloalkyl may include multiple fused and/or bridged rings. Non-limiting examples of fused/bridged cycloalkyl includes: bicyclo[1.1.0]butanyl, bicyclo[2.1.0]pentanyl, bicyclo[1.1.1]pentanyl, bicyclo[3.1.0]hexanyl, bicyclo[2.1.1]hexanyl, bicyclo[3.2.0]heptanyl, bicyclo[4.1.0]heptanyl, bicyclo[2.2.1]heptanyl, bicyclo[3.1.1]heptanyl, bicyclo[4.2.0]octanyl, bicyclo[3.2.1]octanyl, bicyclo[2.2.2]octanyl, and the like. Cycloalkyl also includes spirocyclic rings (e.g., spirocyclic bicycle wherein two rings are connected through just one atom). Non-limiting examples of spirocyclic cycloalkyls include spiro[2.2]pentanyl, spiro[2.5]octanyl, spiro[3.5]nonanyl, spiro[3.5]nonanyl, spiro[3.5]nonanyl, spiro[4.4]nonanyl, spiro[2.6]nonanyl, spiro[4.5]decanyl, spiro[3.6]decanyl, spiro[5.5]undecanyl, and the like. The term “saturated” as used in this context means only single bonds present between constituent carbon atoms.

The term “cycloalkenyl” as used herein means partially unsaturated cyclic hydrocarbon groups having 3 to 20 ring carbons, preferably 3 to 16 ring carbons, and more preferably 3 to 12 ring carbons or 3-10 ring carbons or 3-6 ring carbons, wherein the cycloalkenyl group may be optionally substituted. Examples of cycloalkenyl groups include, without limitation, cyclopentenyl, cyclohexenyl, cycloheptenyl, and cyclooctenyl. As partially unsaturated cyclic hydrocarbon groups, cycloalkenyl groups may have any degree of unsaturation provided that one or more double bonds is present in the ring, none of the rings in the ring system are aromatic, and the cycloalkenyl group is not fully saturated overall. Cycloalkenyl may include multiple fused and/or bridged and/or spirocyclic rings.

The term “heteroaryl”, as used herein, means a mono-, bi-, tri- or polycyclic group having 5 to 20 ring atoms, alternatively 5, 6, 9, 10, or 14 ring atoms; and having 6, 10, or 14 pi electrons shared in a cyclic array; wherein at least one ring in the system is aromatic, and at least one ring in the system contains one or more heteroatoms independently selected from the group consisting of N, O, and S (but does not have to be a ring which contains a heteroatom, e.g. tetrahydroisoquinolinyl, e.g., tetrahydroquinolinyl). Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridinyl, pyrazolo[4,3-b]pyridinyl, tetrazolyl, chromanyl, 2,3-dihydrobenzo[b][1,4]dioxinyl, benzo[d][1,3]dioxolyl, benzo[d]thiazolyl, 2,3-dihydrobenzofuran, tetrahydroquinolinyl, 2,3-dihydrobenzo[b][1,4]oxathiinyl, indolinyl, isoindolinyl, and others. In some embodiments, the heteroaryl is selected from thienyl, pyridinyl, furyl, pyrazolyl, imidazolyl, isoindolinyl, pyranyl, pyrazinyl, and pyrimidinyl.

The term “heterocyclyl” refers to a mon-, bi-, tri-, or polycyclic saturated ring system with 3-16 ring atoms (e.g., 5-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, and the like. Heterocyclyl may include multiple fused and bridged rings. Non-limiting examples of fused/bridged heteorocyclyl includes: 2-azabicyclo[1.1.0]butanyl, 2-azabicyclo[2.1.0]pentanyl, 2-azabicyclo[1.1.1]pentanyl, 3-azabicyclo[3.1.0]hexanyl, 5-azabicyclo[2.1.1]hexanyl, 3-azabicyclo[3.2.0]heptanyl, octahydrocyclopenta[c]pyrrolyl, 3-azabicyclo[4.1.0]heptanyl, 7-azabicyclo[2.2.1]heptanyl, 6-azabicyclo[3.1.1]heptanyl, 7-azabicyclo[4.2.0]octanyl, 2-azabicyclo[2.2.2]octanyl, 3-azabicyclo[3.2.1]octanyl, 2-oxabicyclo[1.1.0]butanyl, 2-oxabicyclo[2.1.0]pentanyl, 2-oxabicyclo[1.1.1]pentanyl, 3-oxabicyclo[3.1.0]hexanyl, 5-oxabicyclo[2.1.1]hexanyl, 3-oxabicyclo[3.2.0]heptanyl, 3-oxabicyclo[4.1.0]heptanyl, 7-oxabicyclo[2.2.1]heptanyl, 6-oxabicyclo[3.1.1]heptanyl, 7-oxabicyclo[4.2.0]octanyl, 2-oxabicyclo[2.2.2]octanyl, 3-oxabicyclo[3.2.1]octanyl, and the like. Heterocyclyl also includes spirocyclic rings (e.g., spirocyclic bicycle wherein two rings are connected through just one atom). Non-limiting examples of spirocyclic heterocyclyls include 2-azaspiro[2.2]pentanyl, 4-azaspiro[2.5]octanyl, 1-azaspiro[3.5]nonanyl, azaspiro[4.4]nonanyl, 2-azaspiro[3.5]nonanyl, 7-azaspiro[3.5]nonanyl, 2-6-azaspiro[2.6]nonanyl, 1,7-diazaspiro[4.5]decanyl, 7-azaspiro[4.5]decanyl, 2,5-diazaspiro[3.6]decanyl, 3-azaspiro[5.5]undecanyl, 2-oxaspiro[2.2]pentanyl, 4-oxaspiro[2.5]octanyl, 1-oxaspiro[3.5]nonanyl, 2-oxaspiro[3.5]nonanyl, 7-oxaspiro[3.5]nonanyl, 2-oxaspiro[4.4]nonanyl, 6-oxaspiro[2.6]nonanyl, 1,7-dioxaspiro[4.5]decanyl, 2,5-dioxaspiro[3.6]decanyl, 1-oxaspiro[5.5]undecanyl, 3-oxaspiro[5.5]undecanyl, 3-oxa-9-azaspiro[5.5]undecanyl and the like. The term “saturated” as used in this context means only single bonds present between constituent ring atoms and other available valences occupied by hydrogen and/or other substituents as defined herein.

The term “heterocycloalkenyl” as used herein means partially unsaturated cyclic ring system with 3-16 ring atoms (e.g., 5-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocycloalkenyl groups include, without limitation, tetrahydropyridyl, dihydropyrazinyl, dihydropyridyl, dihydropyrrolyl, dihydrofuranyl, dihydrothiophenyl. As partially unsaturated cyclic groups, heterocycloalkenyl groups may have any degree of unsaturation provided that one or more double bonds is present in the ring, none of the rings in the ring system are aromatic, and the heterocycloalkenyl group is not fully saturated overall. Heterocycloalkenyl may include multiple fused and/or bridged and/or spirocyclic rings.

As used herein, when a ring is described as being “aromatic”, it means said ring has a continuous, delocalized π-electron system. Typically, the number of out of plane π-electrons corresponds to the Hückel rule (4n+2). Examples of such rings include: benzene, pyridine, pyrimidine, pyrazine, pyridazine, pyridone, pyrrole, pyrazole, oxazole, thioazole, isoxazole, isothiazole, and the like.

As used herein, when a ring is described as being “partially unsaturated”, it means said ring has one or more additional degrees of unsaturation (in addition to the degree of unsaturation attributed to the ring itself; e.g., one or more double or triple bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

For the avoidance of doubt, and unless otherwise specified, for rings and cyclic groups (e.g., aryl, heteroaryl, heterocyclyl, heterocycloalkenyl, cycloalkenyl, cycloalkyl, and the like described herein) containing a sufficient number of ring atoms to form bicyclic or higher order ring systems (e.g., tricyclic, polycyclic ring systems), it is understood that such rings and cyclic groups encompass those having fused rings, including those in which the points of fusion are located (i) on adjacent ring atoms (e.g., [x.x.0] ring systems, in which 0 represents a zero atom bridge

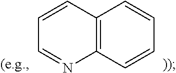

(ii) a single ring atom (spiro-fused ring systems)

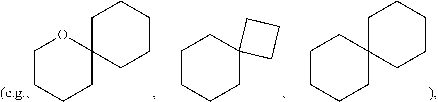

or (iii) a contiguous array of ring atoms (bridged ring systems having all bridge lengths>0)

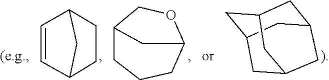

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In addition, the compounds generically or specifically disclosed herein are intended to include all tautomeric forms. Thus, by way of example, a compound containing the moiety:

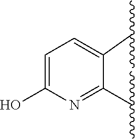 [SMILES: *c1ccc(O)nc1*]

encompasses the tautomeric form containing the moiety:

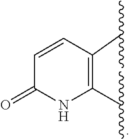 [SMILES: *c1ccc(=O)[nH]c1*]

Similarly, a pyridinyl or pyrimidinyl moiety that is described to be optionally substituted with hydroxyl encompasses pyridone or pyrimidone tautomeric forms. As a further non-limiting example, a compound containing the moiety:

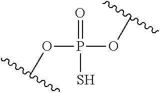 [SMILES: *OP(=O)(S)O*]

encompasses the tautomeric form containing the moiety:

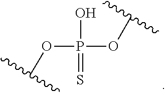 [SMILES: *OP(O)(=S)O*]

The details of one or more embodiments of this disclosure are set forth in the description below. Other features and advantages of the invention will be apparent from the description, and from the claims.

### DETAILED DESCRIPTION

This disclosure features chemical entities (e.g., a compound that modulates (e.g., agonizes) alpha kinase 1 (ALPK1), or a pharmaceutically acceptable salt, and/or hydrate, and/or cocrystal, and/or tautomer, and/or stereoisomer, and/or stable isotope, and/or prodrug, and/or drug combination of the compound) that are useful, e.g., for treating a condition, disease or disorder in which a decrease or increase in ALPK1 activity (e.g., a decrease, e.g., a condition, disease or disorder associated with repressed or impaired ALPK1 signaling) contributes to the pathology and/or symptoms and/or progression of the condition, disease or disorder (e.g., cancer; or e.g., immune and/or inflammatory related diseases (e.g., IBD)) in a subject (e.g., a human). This disclosure also features compositions as well as other methods of using and making the same.

The chemical entities described herein can promote systemic immune response and/or cytokine production. In addition, the chemical entities can serve as vaccine adjuvant with promotion of OVA (ovalbumin)-specific immunoglobulin (IgG).

### DETAILED DESCRIPTION OF THE EMBODIMENTS

In one aspect, the present disclosure provides compounds represented by Formula (X):

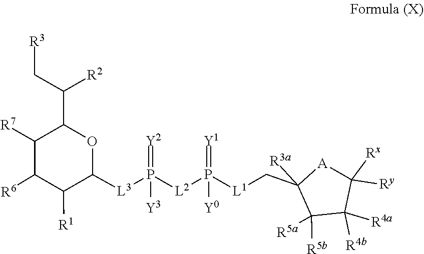

or a pharmaceutically acceptable salt, a stereoisomer, stable isotope, prodrug, or a tautomer thereof, wherein:
R^{X} is:
(A) a moiety having formula (X-Ia), (X-Ib), or (X-Ic):

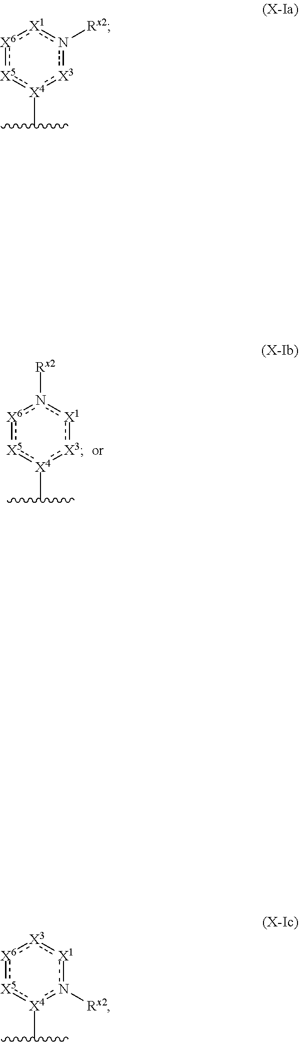

wherein:
X¹ is selected from the group consisting of: C(═O), C—OH, C═S, C—SH, C—NH₂, and C(═NH);
X³, X⁵, and X⁶ are each independently selected from the group consisting of: N, NH, N(R^{Xn}), CH, CR^{Xc}, C(═O), C(═S), C(═NH), and C(═NR^{Xn});
X⁴ is N or C;
R^{X2} is —H, R^{Xn}, or is absent when a double bond is present between NR^{X2} and an adjacent ring atom; and
each occurrence of [symbol] is independently a single bond or a double bond;
   provided that formulas (X-Ia), (X-Ib), and (X-Ic) each include from 1-2 endocyclic double bonds;
   provided that when X⁴ is C, then a double bond is present between X⁴ and an adjacent ring atom; and
   provided that when formulas (X-Ia), (X-Ib), and (X-Ic) each include only 1 endocyclic double bond, then X⁴ is N and/or one or more of X³, X⁵, and X⁶ are each independently selected from the group consisting of: N, NH, N(R^{Xn}), C(═O), C(═S), C(═NH), and C(═NR^{Xn});
(B) pyridinyl, pyrmidinyl, pyrazinyl, pyridazinyl, or triazinyl each of which is optionally substituted with from 1-3 R^{Xc}, provided that any R^{Xc} group that is ortho or para to a ring nitrogen of (B) is other than —OH, —SH, or NH₂;
(C) a moiety having formula (X-II):

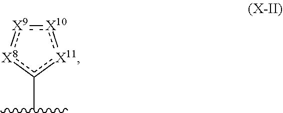

wherein:
X⁷ is C or N;
X⁸, X⁹, X¹⁰, and X¹¹ are each independently selected from the group consisting of: CH, C(R^{Xc}), N, N(H), N(R^{Xn}), O, S, C(═O), C(═S), C(═NH), and C(═NR^{Xn}); and
each [symbol] is independently a single bond or a double bond,
provided that from 1-4 of X⁷—X¹¹ is independently selected from group consisting of C, CH, C(R^{Xc}), C(═O), C(═S), C(═NH), and C(═NR^{Xn}), and (X-II) is aromatic;
(D) C₆₋₁₀ aryl optionally substituted with from 1-4 R^{Xc}; or
(E) bicyclic heteroaryl having 8-12 ring atoms, wherein from 1-5 ring atoms are heteroatoms each independently selected from the group consisting of: N, N(H), N(R^{Xn}), O, and S(═O)₀₋₂, and wherein one or more ring carbon atoms of the heteroaryl is optionally substituted with from 1-4 substituents each independently selected from the group consisting of oxo and R^{Xc}.
each occurrence of R^{Xc} is independently selected from the group consisting of: R^{c}, R^{b}, and -(L^{b})_{b}-R^{b};
each occurrence of R^{Xn} is independently selected from the group consisting of: R^{d}, R^{b}, and -(L^{b})_{b}-R^{b};
R^{Y}, R^{4a}, R^{4b}, R^{5a}, and R^{5b} are each independently selected from the group consisting of:
   H, —OH, —SH, -halo, cyano, or azido;
   C₁₋₆ alkyl, C₁₋₆ haloalkyl, C₂₋₆ alkenyl, C₂₋₆ haloalkenyl, C₂₋₆ alkynyl, or C₂-6 haloalkynyl, each of which is optionally substituted with from 1-6 R^{a};
   C₁₋₄ alkoxy or C₁₋₄ thioalkoxy, each of which is optionally substituted with from 1-6 R^{a};
   —OR⁹, —NR^{e}R^{f};
   —R^{b} or -(L^{b})_{b}-R^{b};
   —OP(═O)(OR′)(OR″); and
   —OC(═O) (C₁₋₆ alkyl) optionally substituted with from 1-6 R^{a}; or
L¹, L², L³ and A are each independently selected from the group consisting of: —O—, —S—, —NR^{L1}—, and —C(R^{L2}) (R^{L2})—;
Y¹ and Y² are each independently selected from the group consisting of: O and S;
Y⁰ and Y³ are each independently selected from the group consisting of: —OH, —OR⁹, —SH, and —SR⁹,
R¹, R², R⁶, and R⁷ are each independently selected from the group consisting of: H, D, -halo, —OH, —SH, cyano, —OR⁹, —OC(═O)R⁹, —NR^{e}R^{f}, —NR^{e}C(═O)R⁹, —OP(═O)(OR′)(OR″), —OS(═O)₁₋₂R⁹, C₁₋₆ alkyl, C₁₋₆ haloalkyl, and —OR⁸;
R³ is selected from the group consisting of: H, D, -halo, —OH, —SH, cyano, —C(═O)OH, —C(═O)O(C₁₋₄ alkyl), —C(═O)NR′R″, —OR¹⁰, —OC(═O)R¹⁰, —NR^{e}R^{f}, —NR^{e}C(═O)R¹⁰, —OP(═O)(OR′)(OR″), —OS(═O)₁₋₂R¹⁰, C₁₋₆ alkyl, C₁₋₆ haloalkyl, and —OR⁸;
R^{3a} is selected from the group consisting of: —OH, —SH, —H, -halo, cyano, C₁₋₆ alkyl, C₁₋₆ haloalkyl, —C(═O)OH, —C(═O)O(C₁₋₄ alkyl), —C(═O)NR′R″, —OP(═O)(OR′)(OR″), C₁₋₄ alkoxy, C₁₋₄ haloalkoxy, —OR⁸, and —NR^{e}R^{f};
each occurrence of R⁸ is independently selected from the group consisting of:
   —C(═O) C₁₋₂₀ alkyl optionally substituted with from 1-10 substituents independently selected from the group consisting of: R^{a}, R^{b}, and -(L^{b})_{b}-R^{b};
   C(═O)—(R^{b2})ₘ₁—R^{8b}, wherein each R^{b2} is independently a divalent R^{b} group, m1 is an integer from 1 to 6, and R^{8b} is —H or R^{c};

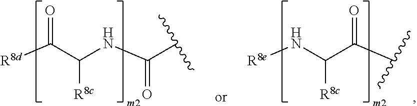

wherein:
m2 is an integer from 1 to 10;
each R^{8c} is independently selected from the group consisting of: —H; C₁₋₆ alkyl, which is optionally substituted with from 1-4 R^{a}; —R^{b}; and —(C₁₋₆ alkylene)-R^{b};
R^{8d} is selected from the group consisting of: —H, —OH, —C₁₋₄ alkoxy, and NR^{e}R^{f}; and
R^{8e} is selected from the group consisting of: —H, C₁₋₄ alkyl, C(═O) C₁₋₄ alkyl, and C(═O)OC₁₋₄ alkyl;
each occurrence of R⁹ is independently selected from the group consisting of: C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl, 3- to 7-membered heterocyclyl, C₆₋₁₀ aryl, or 5- to 10-membered heteroaryl;
each occurrence of R¹⁰ is independently selected from the group consisting of: C₁₋₂₀ alkyl, C₁₋₂₀ haloalkyl, C₂₋₂₀ alkenyl, C₂₋₂₀ haloalkenyl, C₂₋₂₀ alkynyl, C₂₋₂₀ haloalkynyl, C₃₋₇ cycloalkyl, 3- to 7-membered heterocyclyl, C₆₋₁₀ aryl, or 5- to 10-membered heteroaryl;
each occurrence of R^{L1} is independently selected from the group consisting of: —H; C₁₋₆ alkyl, C₁₋₆ haloalkyl, C₂₋₆ alkenyl, C₂₋₆ haloalkenyl, C₂₋₆ alkynyl, or C₂₋₆ haloalkynyl, which is optionally substituted with 1-3 substituents each independently selected from the group consisting of NR′R″, —OH, C₁₋₄ alkoxy, and C₁₋₄ haloalkoxy; and —C(═O)R′;
each occurrence of R^{L2} is independently selected from the group consisting of: —H; -halo; —OH; —OR⁹; C₁₋₆ alkyl, C₁₋₆ haloalkyl, C₂₋₆ alkenyl, C₂₋₆ haloalkenyl, C₂₋₆ alkynyl, or C₂₋₆ haloalkynyl, which is optionally substituted with 1-3 substituents each independently selected from the group consisting of NR′R″, —OH, C₁₋₄ alkoxy, and C₁₋₄ haloalkoxy; and —C(═O)R′;
each occurrence of R^{a} is independently selected from the group consisting of: —H, —OH, -halo, —NR^{e}R^{f}, C₁₋₄ alkoxy, C₁₋₄ haloalkoxy, —C(═O)O(C₁₋₄ alkyl), —C(═O) (C₁₋₄ alkyl), —C(═O)OH, —C(═O)NR′R″, —S(═O)₁₋₂NR′R″, —S(═O)₁₋₂ (C₁₋₄ alkyl), and cyano;
each occurrence of R^{b} is independently selected from the group consisting of:
C₃₋₁₀ cycloalkyl or C₃₋₁₀ cycloalkenyl, each of which is optionally substituted with 1-4 R^{c};
heterocyclyl or heterocycloalkenyl of 3-10 ring atoms, wherein 1-3 ring atoms are heteroatoms, each independently selected from the group consisting of N, N(H), N(R^{d}), O, and S(═O)₀₋₂, and wherein the heterocyclyl or heterocycloalkenyl is optionally substituted with 1-4 R^{c};
heteroaryl of 5-10 ring atoms, wherein 1-3 ring atoms are heteroatoms, each independently selected from the group consisting of N, N(H), N(R^{d}), O, and S(═O)₀₋₂, and wherein the heteroaryl is optionally substituted with 1-4 R^{c}; and
C₆₋₁₀ aryl optionally substituted with 1-4 R^{c};
each occurrence of L^{b} is independently selected from the group consisting of: —O—, —NH—, —NR^{d}, —S(═O)₀₋₂, C(═O), and C₁₋₃ alkylene optionally substituted with 1-3 R^{a};
each occurrence of b is independently 1, 2, 3, or 4;
each occurrence of R^{e} is independently selected from the group consisting of: halo; cyano; C₁₋₁₀ alkyl, which is optionally substituted with 1-6 independently selected R^{a}; C₂₋₆ alkenyl; C₂₋₆ alkynyl; C₁₋₄ alkoxy; C₁₋₄ haloalkoxy; —S(═O)₁₋₂ (C₁₋₄ alkyl); —NR^{e}R^{f}; —OH; —SH; —S(═O)₁₋₂NR′R″; —C₁₋₄ thioalkoxy; —NO₂; —OC(═O) (C₁₋₄ alkyl); —OC(═O) H; —C(═O) (C₁₋₄ alkyl); —C(═O) H; —C(═O)O(C₁₋₄ alkyl); —C(═O)OH; and —C(═O)NR′R″;
each occurrence of R^{d} is independently selected from the group consisting of: C₁₋₆ alkyl optionally substituted with 1-3 independently selected R^{a}; —C(═O) (C₁₋₄ alkyl); —C(═O)O(C₁₋₄ alkyl); —C(═O)NR′R″; —S(═O)₁₋₂NR′R″; —S(═O)₁₋₂ (C₁₋₄ alkyl); —OH; and C₁₋₄ alkoxy;
each occurrence of R^{e} and R^{f} is independently selected from the group consisting of: —H; C₁₋₆ alkyl or C₁₋₆ haloalkyl, which is optionally substituted with 1-3 substituents each independently selected from the group consisting of NR′R″, —OH, halo, C₁₋₄ alkoxy, and C₁₋₄ haloalkoxy; —C(═O)R′; —C(═O)OR′; —C(═O)NR′R″; C(═NR″)NR′R″; —C(═O) C(═O)R′; —S(═O)₁₋₂NR′R″; —S(═O)₁₋₂R′; —OH; and C₁₋₄ alkoxy; or
R^{e}, and R^{f} taken together with the N atom connecting them form a saturated or unsaturated 3- to 7-membered heterocyclyl; and
each occurrence of R′ and R″ is independently selected from the group consisting of: —H; C₁₋₄ alkyl or C₁₋₄ haloalkyl, which is optionally substituted with 1-3 substituents each independently selected from the group consisting of: halo, cyano, C₁₋₄ alkoxy, C₁₋₄ haloalkoxy, and —OH;
provided that at least one of the following is true:
a) R^{4a} is selected from the group consisting of: C₂₋₆ alkenyl, C₂₋₆ haloalkenyl, C₂₋₆ alkynyl, and C₂₋₆ haloalkynyl;
b) R^{4b} is NR^{e}R^{f}.

### Variable R^{X}

In some embodiments as mentioned above, R^{X} is (A) a moiety having formula

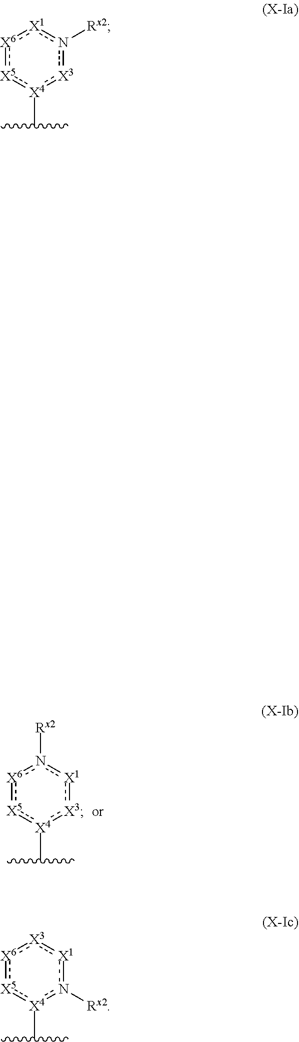

In some embodiments as mentioned above, R^{X} is

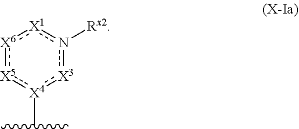

In some embodiments as mentioned above (when R^{X} is (X-Ia) or Formula (I-1), X¹ is C(═O) or C—OH. For example, X¹ can be C(═O).

In some embodiments as mentioned above (when R^{X} is (X-Ia) or Formula (I-1), X¹ is C(═NH) or C—NH₂. For example, X¹ can be C—NH₂.

In some embodiments as mentioned above (when R^{X} is (X-Ia) or Formula (I-1), X³ is C(═O).

In some embodiments as mentioned above (when R^{X} is (X-Ia) or Formula (I-1), X⁴ is N. In certain embodiments of Formula (I) (when R^{X} is (X-Ia) or Formula (I-1), X³ is C(═O); and X⁴ is N.

In some embodiments as mentioned above (when R^{X} is (X-Ia) or Formula (I-1), R^{X2} is —H or absent. In certain embodiments of Formula (I) (when R^{X} is (X-Ia) or Formula (I-1), X¹ is C(═O); and R^{X2} is —H. In certain embodiments, X¹ is C—NH₂; and R^{X2} is absent. In certain of these embodiments, X³ is C(═O); and X⁴ is N.

In some embodiments as mentioned above (when R^{X} is (X-Ia) or Formula (I-1), X⁵ and X⁶ are each independently CH or CR^{Xc}, such as CH or CR^{c}.

In some embodiments as mentioned above (when R^{X} is (X-Ia) or Formula (I-1), R^{X} is selected from the group consisting of:

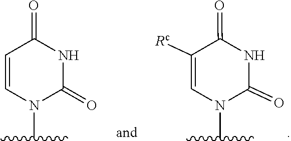

In certain of these embodiments, R^{X} is selected from the group consisting of:

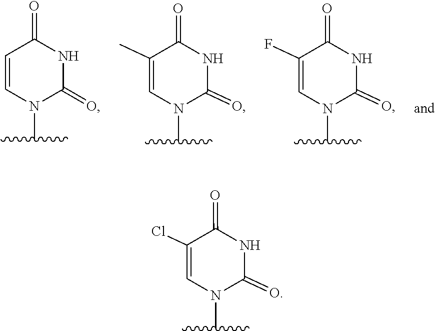

In some embodiments as mentioned above (when R^{X} is (X-Ia) or Formula (I-1), R^{X} is selected from the group consisting of:

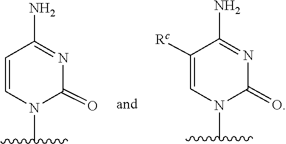

In certain of these embodiments, R^{X} is selected from the group consisting of:

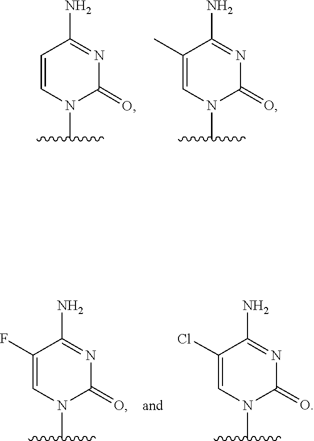

In some embodiments as mentioned above, R^{X} is: (B) pyridinyl, pyrmidinyl, pyrazinyl, pyridazinyl, or triazinyl each of which is optionally substituted with from 1-3 R^{Xc}, provided that any R^{Xc} group that is ortho or para to a ring nitrogen of (B) is other than —OH, —SH, or NH₂.

In certain embodiments, R^{X} is

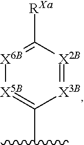

wherein R^{Xa} is selected from the group consisting of:
—OH, —C₁₋₄ alkoxy, —C₁₋₄ haloalkoxy, —OR^{b}, or —O—(C₁₋₃ alkylene)-R^{b},
—NR^{e}R^{f}, —NHR^{b}, or —NH—(C₁₋₃ alkylene)-R^{b};
—C(O)NR′R″, —C(O)NHR^{b}, or —C(O)NH—(C₁₋₃ alkylene)-R^{b};
—C(O)OC₁₋₄ alkyl, —C(O)OH, —C(═O)OR^{b}, or —C(═O)O—(C₁₋₃ alkylene)-R^{b};
—OC(O) C₁₋₄ alkyl, —OC(═O)R^{b}, or —OC(═O)—(C₁₋₃ alkylene)-R^{b}; and
—NHC(═O)R^{b} or —NHC(═O)—(C₁₋₃ alkylene)-R^{b};
X^{2B}, X^{3B}, X^{5B}, and X^{6B} are each independently N, CH, or CR^{Xc}, provided that from 1-3 of X^{2B}, X^{3B}, X^{5B}, and X^{6B} is CH; and from 1-2 of X^{2B}, X^{3B}, X^{5B}, and X^{6B} is N, further provided that when one or both of X^{2B} and X^{6B} is N, then R^{Xa} is other than —OH or NH₂.

In some embodiments as mentioned above, R^{X} is

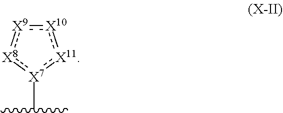

In certain of these embodiments, X¹⁰ is CR^{Xc}.

In certain of the foregoing embodiments, R^{X} is

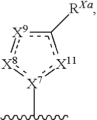

wherein: X⁷ is N or C; each of X⁸, X⁹, and X¹¹ is independently selected from the group consisting of: N, N(H), N(R^{Xn}), CH, CR^{Xc}, O, and S; and
R^{Xa} is selected from the group consisting of:
OH, —C₁₋₄ alkoxy, —C₁₋₄ haloalkoxy, -ORD, or —O—(C₁₋₃ alkylene)-R^{b};
—NR^{e}R^{f}, —NHR^{b}, or —NH—(C₁₋₃ alkylene)-R^{b};
—C(O)NR′R″, —C(O)NHR^{b}, or —C(O)NH—(C₁₋₃ alkylene)-R^{b};
—C(O)OH, —C(O)OC₁₋₄ alkyl, —C(═O)OR^{b}, or —C(═O)O—(C₁₋₃ alkylene)-R^{b};
—OC(O) C₁₋₄ alkyl, —OC(═O)R^{b}, or —OC(═O)—(C₁₋₃ alkylene)-R^{b}; and
—NHC(═O)R^{b}, or —NHC(═O)—(C₁₋₃ alkylene)-R^{b}.

In certain embodiments (when R^{X} is (X-II)), X⁷ is N.

In certain embodiments (when R^{X} is (X-II)), X¹¹ is N or CH.

In certain other embodiments (when R^{X} is (X-II)), X¹¹ is CR^{Xc}, wherein X¹¹ is other than C—NH₂.

R^{Xa} is selected from the group consisting of: —C(O)NR′R″, —C(O)NHR^{b}, and —C(O)NH—(C₁₋₃ alkylene)-R^{b}.

In certain of the foregoing embodiments, R^{Xa} is C(O)NR′R″, such as wherein R^{Xa} is —C(O)NH₂.

As non-limiting examples when R^{X} is (X-II), R^{X} can be selected from the group consisting of:

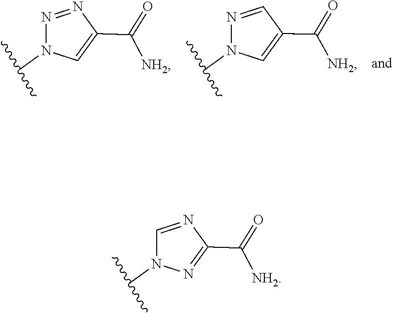

In some embodiments as mentioned above, R^{X} is C₆₋₁₀ aryl optionally substituted with from 1-4 R^{Xc}. In certain of these embodiments, R^{X} is phenyl which is substituted with from 1-4R^{Xc}.

In certain of the foregoing embodiments, R^{X} is phenyl that is substituted with R^{Xa} and further optionally substituted with from 1-2 R^{c}, wherein:
R^{Xa} is selected from the group consisting of:
—OH, —C₁₋₄ alkoxy, —C₁₋₄ haloalkoxy, —OR^{b}, or —O—(C₁₋₃ alkylene)-R^{b};
—NR^{e}R^{f}, —NHR^{b}, or —NH—(C₁₋₃ alkylene)-R^{b};
—C(O)NR′R″, —C(O)NHR^{b}, or —C(O)NH—(C₁₋₃ alkylene)-R^{b};
—C(O)OH, —C(O)OC₁₋₄ alkyl, —C(═O)OR^{b}, or —C(═O)O—(C₁₋₃ alkylene)-R^{b};
—OC(O) C₁₋₄ alkyl, —OC(═O)R′, or —OC(═O)—(C₁₋₃ alkylene)-R²; and
—NHC(═O)R′, or —NHC(═O)—(C₁₋₃ alkylene)-R′.

In certain embodiments of Formula (I), R^{X} is

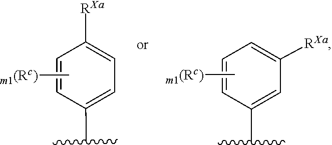

wherein m1 is 0, 1, or 2.

In certain embodiments of Formula (I), R^{X} is

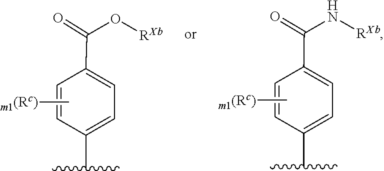

wherein R^{Xb} is —H, C₁₋₄ alkyl, R^{b}, or —(C₁₋₃ alkylene)-R^{b}; and m1 is 0, 1, or 2.

In certain embodiments of Formula (I), R^{X} is

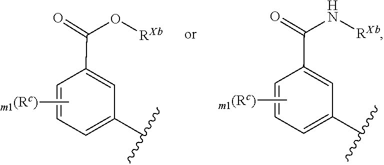

wherein R^{Xb} is —H, C₁₋₄ alkyl, R^{b}, or —(C₁₋₃ alkylene)-R^{b}; and m1 is 0, 1, or 2.

In certain embodiments of Formula (I), R^{X} is

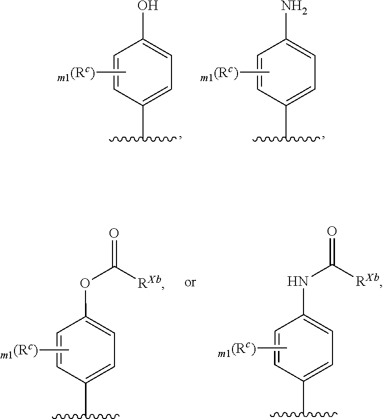

wherein R^{Xb} is —H, C₁₋₄ alkyl, R^{b}, or —(C₁₋₃ alkylene)-R^{b}; and m1 is 0, 1, or 2.

In certain embodiments of Formula R^{X} is

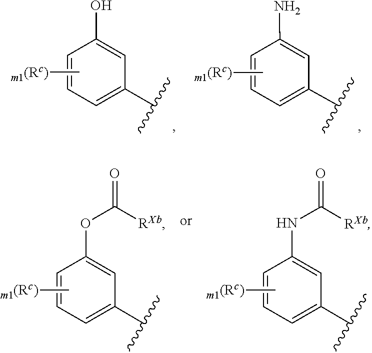

wherein R^{Xb} is —H, C₁₋₄ alkyl, R^{b}, or —(C₁₋₃ alkylene)-R^{b}; and m1 is 0, 1, or 2.

In some embodiments as mentioned above, R^{X} is bicyclic heteroaryl having 8-12 ring atoms, wherein from 1-5 ring atoms are heteroatoms each independently selected from the group consisting of: N, N(H), N(R^{Xn}), O, and S(O)₀₋₂, and wherein one or more ring carbon atoms of the heteroaryl is optionally substituted with from 1-4 substituents each independently selected from the group consisting of oxo and R^{Xc}.

In certain of these embodiments, R^{X} is bicyclic heteroaryl having 9-10 (e.g., 9) ring atoms, wherein from 1-5 ring atoms are heteroatoms each independently selected from the group consisting of: N, N(H), N(R^{Xn}), O, and S(O)₀₋₂, and wherein one or more ring carbon atoms of the heteroaryl is optionally substituted with from 1-4 substituents each independently selected from the group consisting of oxo and R^{Xc}.

In some embodiments as mentioned above, R^{X} is

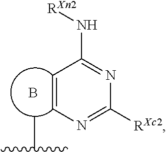

wherein: Ring B is heteroaryl having 5 ring atoms wherein from 1-3 ring atoms are heteroatoms each independently selected from the group consisting of: N, N(H), N(R^{Xn}), O, and S, and wherein Ring B is optionally substituted with R^{Xc};
R^{Xn2} is —H or R^{Xn} (e.g., —H); and
R^{Xc2} is —H or R^{Xc} (e.g., —H).

In certain of these embodiments, R^{Xn2} is —H. In certain of the foregoing embodiments, R^{Xc2} is —H.

In certain of the foregoing embodiments, R^{X} is selected from the group consisting of:

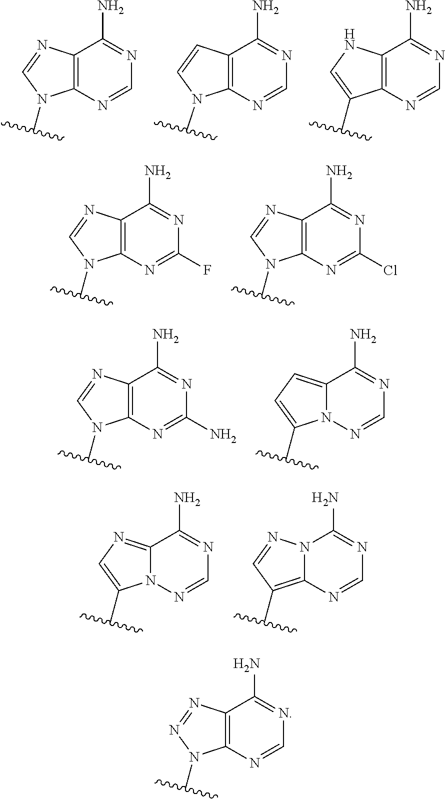

For example, R^{X} can be

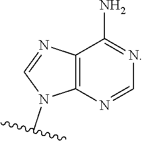 [SMILES: *n1cnc2c(N)ncnc21]

In some embodiments as mentioned above, R^{X} is selected from the group consisting:

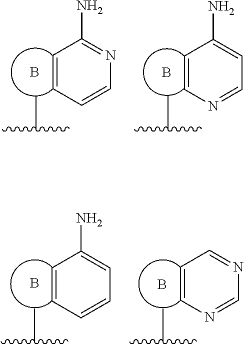

wherein: Ring B is heteroaryl having 5 ring atoms wherein from 1-2 ring atoms are heteroatoms each independently selected from the group consisting of: N, N(H), N(R^{Xn}), O, and S, and wherein Ring B is optionally substituted with R^{Xc}.

For example, R^{X} can be selected from the group consisting of:

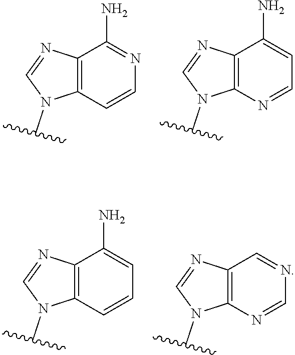

In some embodiments as mentioned above, R^{X} is selected from the group consisting:

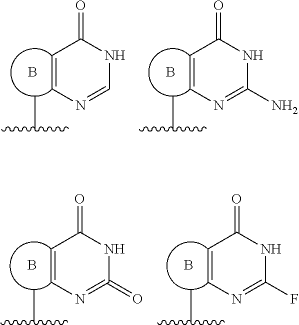

wherein: Ring B is heteroaryl having 5 ring atoms wherein from 1-2 ring atoms are heteroatoms each independently selected from the group consisting of: N, N(H), N(R^{Xn}), O, and S, and wherein Ring B is optionally substituted with R^{Xc}.

For example, R^{X} can be selected from the group consisting of:

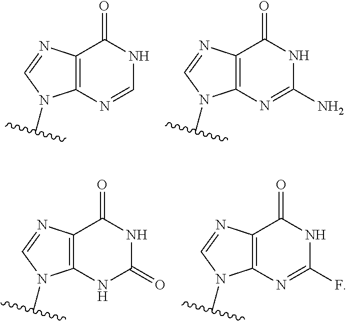 [SMILES: *n1cnc2c(=O)[nH]cnc21 ; *n1cnc2c(=O)[nH]c(N)nc21 ; *n1cnc2c(=O)[nH]c(=O)[nH]c21 ; *n1cnc2c(=O)[nH]c(F)nc21]

Non-limiting examples of R^{X} include:

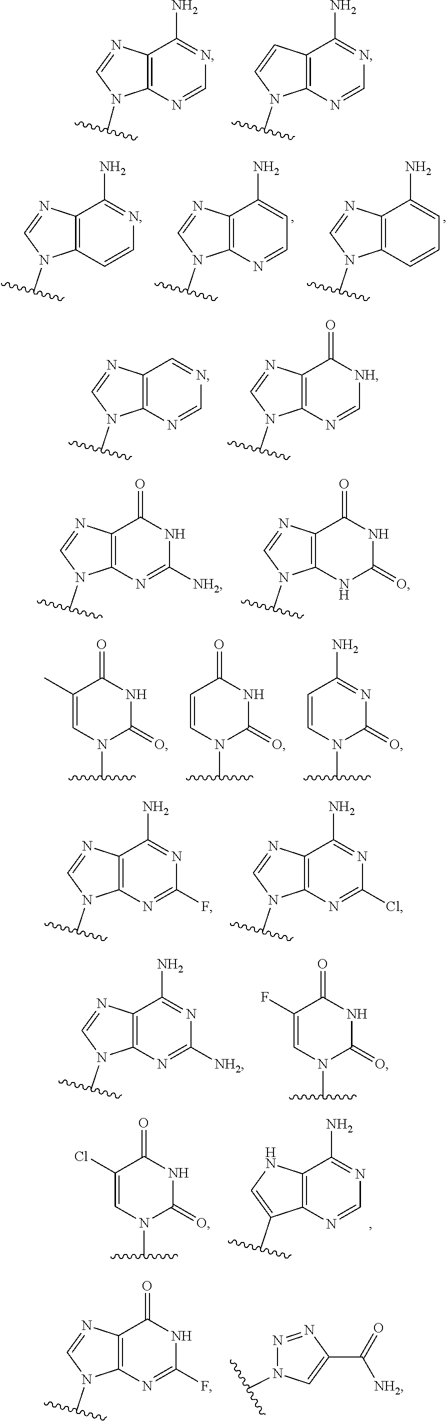

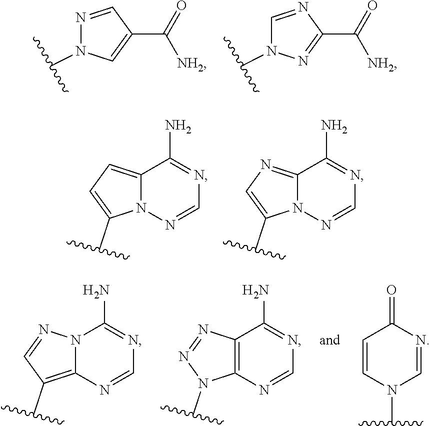

In a specific embodiment of R^{Y}, R^{X} is selected from the group consisting of:

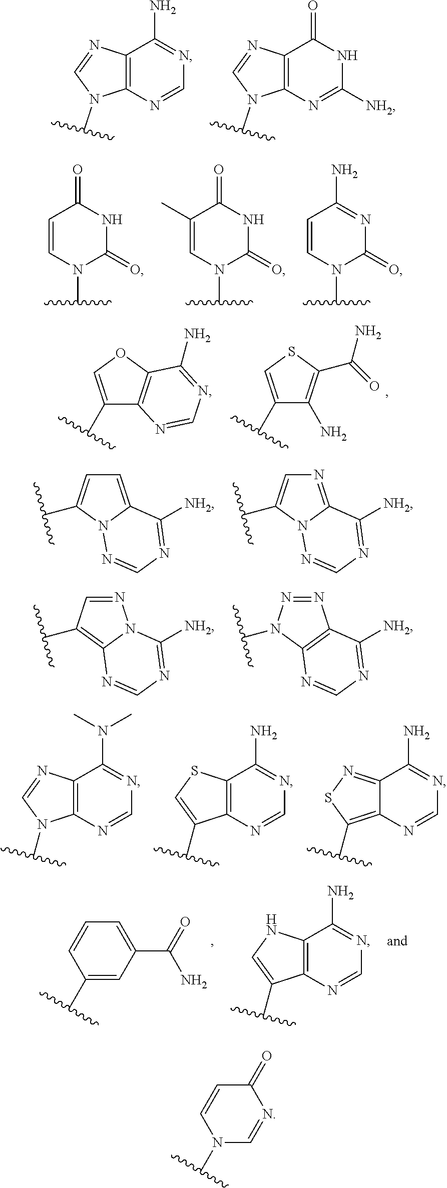

In another specific embodiment of R^{X}, R^{X} is selected from the group consisting of:

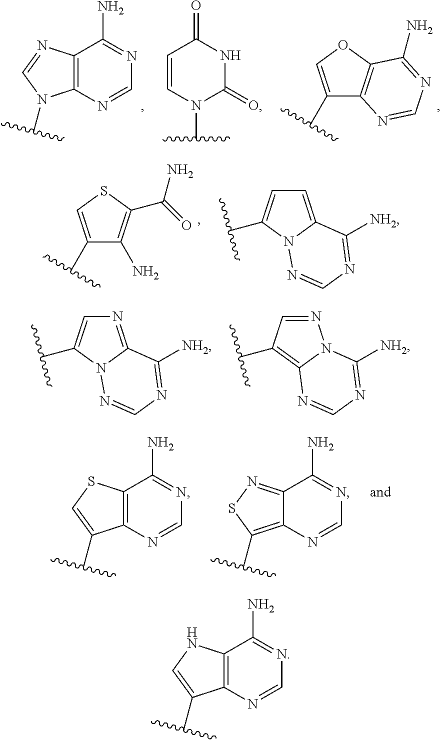

In another specific embodiment of R^{X}, R^{X} is selected from the group consisting of:

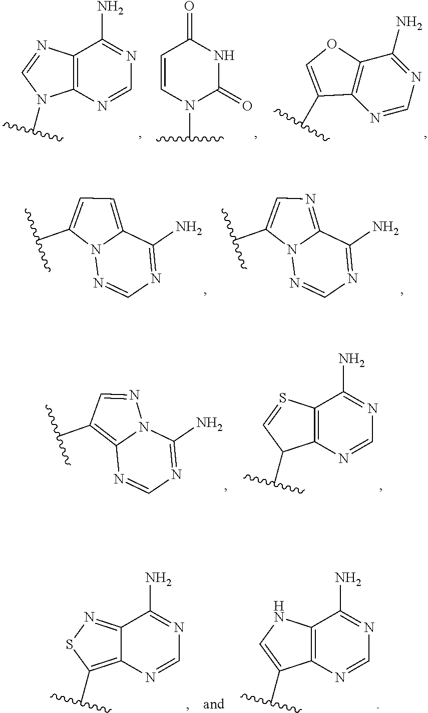

In another specific embodiment of R^{X}, R^{X} is selected from the group consisting of:

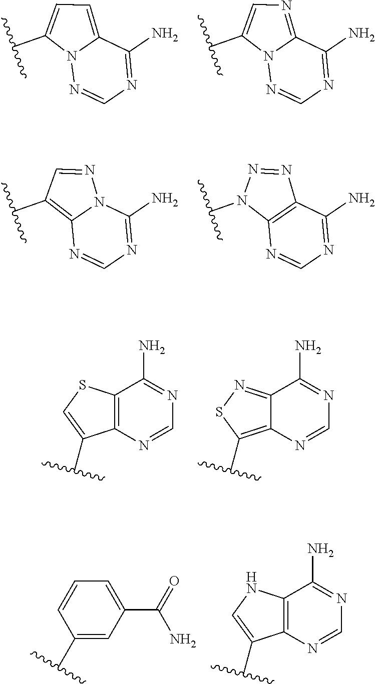

In another specific embodiment of R^{X}, R^{X} is selected from the group consisting of:

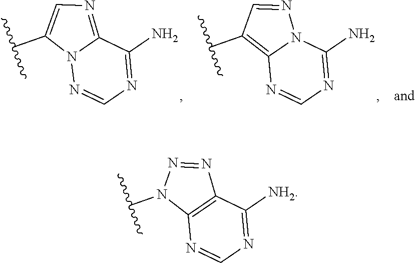 [SMILES: *c1cnc2c(N)ncnn12 ; *c1cnn2c(N)ncnc12 ; *n1nnc2c(N)ncnc21]

In another specific embodiment of R^{X}, R^{X} is selected from the group consisting of:

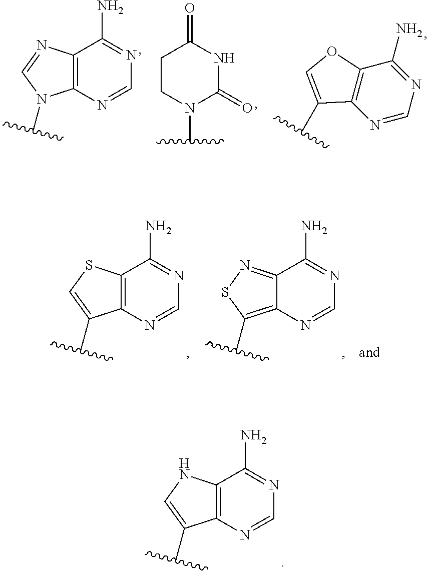

In another specific embodiment of R^{X}, R^{X} is selected from the group consisting of:

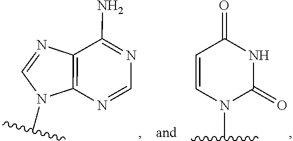 [SMILES: *n1cnc2c(N)ncnc21 ; *n1ccc(=O)[nH]c1=O]

preferably

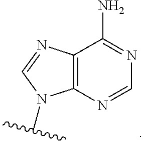 [SMILES: *n1cnc2c(N)ncnc21]

In another specific embodiment of R^{X}, R^{X} is selected from the group consisting of:

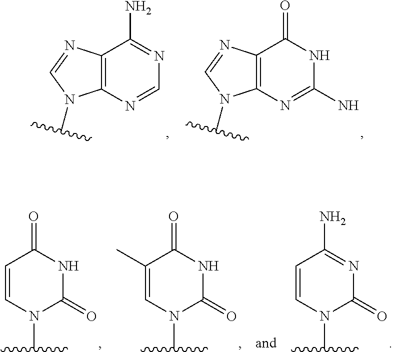

In another specific embodiment of R^{X}, R^{X} is selected from the group consisting of:

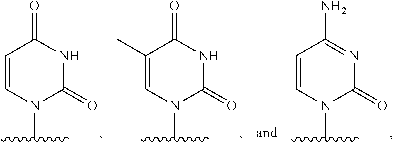

preferably

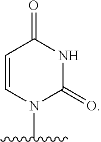 [SMILES: *n1ccc(=O)[nH]c1=O]

In another specific embodiment of R^{X}, R^{X} is

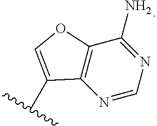 [SMILES: *c1coc2c(N)ncnc12]

In another specific embodiment of R^{X}, R^{X} is

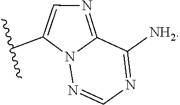 [SMILES: *c1cnc2c(N)ncnn12]

In another specific embodiment of R^{X}, R^{X} is

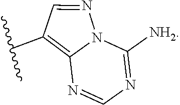 [SMILES: *c1cnn2c(N)ncnc12]

### Variable R^{Y}

In some embodiments as mentioned above, R^{Y} is —H. In some embodiments as mentioned above, R^{Y} is —H, —OH, —SH, -halo, cyano, or azido. In some embodiments as mentioned above, R^{Y} is C₁₋₆ alkyl, C₁₋₆ haloalkyl, C₂₋₆ alkenyl, C₂₋₆ haloalkenyl, C₂₋₆ alkynyl, or C₂₋₆ haloalkynyl, each of which is optionally substituted with from 1-6 R^{a}. In some embodiments as mentioned above, R^{Y} is -halo. In some embodiments as mentioned above, R^{Y} is C₁₋₆ alkyl, or C₁₋₆ haloalkyl. In some embodiments as mentioned above, R^{Y} is C₁₋₆ alkyl.

Variables L¹, L², and L³

In some embodiments as mentioned above, L¹ is —O—.

In some embodiments as mentioned above, L³ is —O—.

In some embodiments as mentioned above, L² is —O—. In some embodiments as mentioned above, L² is —S—. In some embodiments as mentioned above, L² is —NR^{L1}—. In some embodiments as mentioned above, L² is —C(R^{L2}) (R^{L2})—.

### Variable Y⁰

In some embodiments as mentioned above, Y⁰ is —SH.

Variable Y¹, Y², Y³

In some embodiments as mentioned above, Y¹, Y², are O, and Y³ is —OH.

Variable R^{4a}, R^{4b}, R^{5a} and R^{5b}

In some embodiments as mentioned above, R^{4a} is selected from the group consisting of: C₂₋₆ alkenyl, C₂₋₆ haloalkenyl, C₂₋₆ alkynyl, and C₂₋₆ haloalkynyl; and
R^{4b} is selected from the group consisting of:
—H, —OH, —SH, -halo, cyano, or azido;
C₁₋₆ alkyl, C₁₋₆ haloalkyl, C₂₋₆ alkenyl, C₂₋₆ haloalkenyl, C₂₋₆ alkynyl, or C₂₋₆ haloalkynyl, each of which is optionally substituted with from 1-6 R^{a}; C₁₋₄ alkoxy or C₁₋₄ thioalkoxy, each of which is optionally substituted with from 1-6 R^{a};
—OR⁹, —NR^{e}R^{f};
—R^{b} or -(L^{b})_{b}-R^{b};
—OP(═O)(OR′)(OR″); and
—OC(═O) (C₁₋₆ alkyl) optionally substituted with from 1-6 R^{a}.

In some embodiments as mentioned above, R^{4a} is selected from the group consisting of: C₂₋₆ alkenyl, C₂₋₆ haloalkenyl, C₂₋₆ alkynyl, and C₂₋₆ haloalkynyl that containing one to three double bond or triple bond.

In some embodiments as mentioned above, R^{4a} is selected from the group consisting of: C₂₋₆ alkenyl, and C₂₋₆ haloalkenyl that containing cumulated double bonds.

In some embodiments as mentioned above, R^{4a} is selected from the group consisting of: C₂₋₆ alkenyl, and C₂₋₆ haloalkenyl that containing conjugate double bonds.

In some embodiments as mentioned above, R^{4a} is selected from the group consisting of: C₂₋₆ alkenyl, and C₂₋₆ haloalkenyl that containing independent double bonds.

In some embodiments as mentioned above, R^{4a} is selected from the group consisting of: ethenyl, propenyl, ethynyl, and propynyl.

In some embodiments as mentioned above, R^{4a} is selected from the group consisting of: ethenyl, and ethynyl.

In some embodiments as mentioned above, R^{4b} is selected from the group consisting of: —H, —OH, —OR⁹, —OC(═O)R⁹, —NR^{e}R^{f}, and -halo.

In some embodiments as mentioned above, R^{4b} is selected from the group consisting of: —F, —OH, —OR⁹, and —NR^{e}R^{f}.

In some embodiments as mentioned above, R^{4b} is selected from the group consisting of: —F, —OH, —OMe, and —NH₂.

In some embodiments as mentioned above, R^{4b} is selected from the group consisting of: —F, —OH, and —OMe.

In some embodiments as mentioned above, R^{4a} is selected from the group consisting of: C₂₋₆ alkenyl, C₂₋₆ haloalkenyl, C₂₋₆ alkynyl, and C₂₋₆ haloalkynyl; and R^{4b} is selected from the group consisting of: —H, —OH, —OR⁹, —OC(═O)R⁹, —NR^{e}R^{f}, and -halo.

In some embodiments as mentioned above, R^{4a} is selected from the group consisting of: C₂₋₆ alkenyl, C₂₋₆ haloalkenyl, C₂₋₆ alkynyl, and C₂₋₆ haloalkynyl; and R^{4b} is selected from the group consisting of: —OH, —OR⁹, —OC(═O)R⁹, —NR^{e}R^{f}, and -halo.

In some embodiments as mentioned above, R^{4a} is selected from the group consisting of: C₂₋₆ alkenyl, C₂₋₆ haloalkenyl, C₂₋₆ alkynyl, and C₂₋₆ haloalkynyl; and R^{4b} is selected from the group consisting of: —OH, and -halo.

In some embodiments as mentioned above, R^{4a} is C₂₋₆ alkenyl, C₂₋₆ haloalkenyl, C₂₋₆ alkynyl, and C₂₋₆ haloalkynyl; and R^{4b} is selected from the group consisting of: —OH, and —F.

In some embodiments as mentioned above, R^{4b} is —NR^{e}R^{f}; and
R^{4a} and R^{5b} are independently selected from the group consisting of:
H, —OH, —SH, -halo, cyano, or azido;
C₁₋₆ alkyl, C₁₋₆ haloalkyl, C₂₋₆ alkenyl, C₂₋₆ haloalkenyl, C₂₋₆ alkynyl, or C₂₋₆ haloalkynyl, each of which is optionally substituted with from 1-6 R^{a};
C₁₋₄ alkoxy or C₁₋₄ thioalkoxy, each of which is optionally substituted with from 1-6 R^{a};
OR⁹, —NR^{e}R^{f};
—R^{b} or -(L^{b})_{b}-R^{b};
OP(═O)(OR′)(OR″); and
—OC(═O) (C₁₋₆ alkyl) optionally substituted with from 1-6 R^{a}.

In some embodiments as mentioned above, R^{4b} is —NR^{e}R^{f}, and
R^{4a} and R^{5b} are independently selected from the group consisting of:
—H, —OH, —SH, -halo, cyano, or azido;
C₁₋₆ alkyl, C₁₋₆ haloalkyl, C₂₋₆ alkenyl, C₂₋₆ haloalkenyl, C₂₋₆ alkynyl, or C₂₋₆ haloalkynyl, each of which is optionally substituted with from 1-6 R^{a};
C₁₋₄ alkoxy or C₁₋₄ thioalkoxy, each of which is optionally substituted with from 1-6 R^{a};
—OR⁹, —NR^{e}R^{f};
OC(═O) (C₁₋₆ alkyl) optionally substituted with from 1-6 R^{a}.

In some embodiments as mentioned above, R^{4b} is —NR^{e}R^{f}; R^{4a} is selected from the group consisting of: —H, -halo, C₁₋₆ alkyl, C₁₋₆ haloalkyl, C₂₋₆ alkenyl, C₂₋₆ haloalkenyl, C₂₋₆ alkynyl, and C₂₋₆ haloalkynyl; and R^{5b} is independently selected from the group consisting of:
—H, —OH, —SH, -halo, cyano, or azido;
C₁₋₆ alkyl, C₁₋₆ haloalkyl, C₂₋₆ alkenyl, C₂₋₆ haloalkenyl, C₂₋₆ alkynyl, or C₂₋₆ haloalkynyl, each of which is optionally substituted with from 1-6 R^{a};
—OR⁹, —NR^{e}R^{f};

In some embodiments as mentioned above, R^{4b} is —NR^{e}R^{f}; R^{4a} is selected from the group consisting of: —H, -halo, C₁₋₆ alkyl, C₁₋₆ haloalkyl, C₂₋₆ alkenyl, C₂₋₆ haloalkenyl, C₂₋₆ alkynyl, and C₂₋₆ haloalkynyl; and R^{5b} is selected from the group consisting of: —OH, —OR⁹, —NR^{e}R^{f}, and -halo.

In some embodiments as mentioned above, R^{4b} is —NR^{e}R^{f}; R^{4a} is selected from the group consisting of: —H, -halo, C₁₋₆ alkyl, C₁₋₆ haloalkyl, C₂₋₆ alkenyl, C₂₋₆ haloalkenyl, C₂₋₆ alkynyl, and C₂₋₆ haloalkynyl; and R^{5b} is selected from the group consisting of: —OH, —OR⁹, and —NR^{e}R^{f}.

In some embodiments as mentioned above, R^{4b} is —NR^{e}R^{f}; R^{4a} is —H, or Me, preferably —H; and R^{5b} is selected from the group consisting of: —OH, —NH₂, —NHMe, —NMe₂, and —NHAc.

In some embodiments as mentioned above, R^{5b} is —NR^{e}R^{f}; and R^{4b} and R^{5a} are independently selected from the group consisting of:
—H, —OH, —SH, -halo, cyano, or azido;
C₁₋₆ alkyl, C₁₋₆ haloalkyl, C₂₋₆ alkenyl, C₂₋₆ haloalkenyl, C₂₋₆ alkynyl, or C₂₋₆ haloalkynyl, each of which is optionally substituted with from 1-6 R^{a};
C₁₋₄ alkoxy or C₁₋₄ thioalkoxy, each of which is optionally substituted with from 1-6 R^{a};
—OR⁹, —NR^{e}R^{f};
—R^{b} or -(L^{b})_{b}-R^{b};
OP(═O)(OR′)(OR″); and
—OC(═O) (C₁₋₆ alkyl) optionally substituted with from 1-6 R^{a}.
   Variable R¹, R², R³, R⁶, and R⁷

In a specific embodiment, R² is selected from the group consisting of: H; D; -halo; —OH; —SH; cyano; —OR⁹; —OC(═O)R⁹; —NR^{e}R^{f}; —NR^{e}C(═O)R⁹; —OP(═O)(OR′)(OR″); —OS(═O)₁₋₂R⁹; C₁₋₆ alkyl; C₁₋₆ haloalkyl; and —OR⁸.

In another specific embodiment, R² is selected from the group consisting of: H, D, -halo, —OH, —SH, cyano, —OR⁹, —OC(═O)R⁹, —NR^{e}R^{f}, —NR^{e}C(═O)R⁹, —OP(═O)(OR′)(OR″), —OS(═O)₁-2R⁹, C₁₋₆ alkyl, and C₁₋₆ haloalkyl.

In another specific embodiment, R² is selected from the group consisting of: -halo, —OH, —OR⁹, and —OC(═O)R⁹.

In another specific embodiment, R² is selected from the group consisting of: -halo, —OH, or —OC(═O)R⁹.

In another specific embodiment, R² is selected from the group consisting of: —OH, or —OC(═O)R⁹.

In another specific embodiment, R² is selected from the group consisting of: —OH, or —OC(═O) C₁₋₆ alkyl.

In another specific embodiment, R² is selected from the group consisting of: —F, —OH, and —OAc.

In another specific embodiment, R² is selected from the group consisting of: —OH, and OAc.

In another specific embodiment, R² is selected from the group consisting of: —OH; -halo; and —NR^{e}R^{f}.

In another specific embodiment, R² is —OH or NR^{e}R^{f}.

In another specific embodiment, R² is —OH.

In another specific embodiment, R³ is selected from the group consisting of: H; D; -halo; —OH; —SH; cyano; —C(═O)OH; —C(═O)O(C₁₋₄ alkyl); —C(═O)NR′R″; —OR¹⁰; —OC(═O)R¹⁰; —NR^{e}R^{f}; —NR^{e}C(═O)R¹⁰; —OP(═O)(OR′)(OR″); —OS(═O)₁₋₂R¹⁰; C₁₋₆ alkyl; C₁₋₆ haloalkyl; and —OR⁸

In another specific embodiment, R³ is selected from the group consisting of: —OH; —SH; —H; -halo; cyano; —C(═O)OH; —C(═O)O(C₁₋₄ alkyl); —C(═O)NR′R″; —OP(═O)(OR′)(OR″); C₁₋₄ alkoxy; C₁₋₄ haloalkoxy; —OR⁸; and —NR^{e}R^{f}.

In another specific embodiment, R³ is selected from the group consisting of: —OH, halo (e.g., —F), —OP(═O)(OR′)(OR″) (e.g., —OP(═O)(OH) 2), C(═O)OH, NR^{e}R^{f} (e.g., NH₂), —C(═O)NR′R″, and —OR⁸ (e.g., —OC(═O) (C₁₋₄ alkyl).

In another specific embodiment, R³ is selected from the group consisting of: H, D, -halo, —OH, —SH, cyano, —OR¹⁰, —OC(═O)R¹⁰, —NR^{e}R^{f}, —NR^{e}C(═O)R¹⁰, —OP(═O)(OR′)(OR″), —OS(═O)₁₋₂R¹⁰, C₁₋₆ alkyl, and C₁₋₆ haloalkyl.

In another specific embodiment, R³ is selected from the group consisting of: —OH, —OR¹⁰ and —OC(═O)R¹⁰.

In another specific embodiment, R³ is —OH or —OR⁸.

In another specific embodiment, R³ is —OH, or —OC(═O) C₁₋₂₀ alkyl,

In another specific embodiment, R³ is —OH, or —OAc.

In another specific embodiment, R³ is —OH.

In another specific embodiment, R¹, R⁶, and R⁷ are each independently selected from the group consisting of: —OH; —SH; —H; halo; cyano; —NR^{e}Rf; C₁₋₄ alkoxy; C₁₋₄ haloalkoxy; —OP(═O)(OR′)(OR″); and —OR⁸.

In another specific embodiment, R¹, R⁶, and R⁷ are each independently selected from the group consisting of: —OH, —OR⁹, and —OC(═O)R⁹.

In another specific embodiment, R¹, R⁶, and R⁷ are each —OH, or —OC(═O)R⁹.

In another specific embodiment, R¹, R⁶, and R⁷ are each —OH, or —OC(═O) C₁₋₆ alkyl,

In another specific embodiment, R¹, R⁶, and R⁷ are each —OH, or —OAc.

In another specific embodiment, R¹, R⁶, and R⁷ are —OH.

In embodiments, the compound disclosed herein is a compound described in the Examples of this application, such as in Table 1.

The compound of the present disclosure can be prepared using the general processes describes in Schemes 1 to 11 as well as the techniques described in the exemplary embodiments.

In embodiments, the disclosure provides a pharmaceutical composition comprising a compound as described herein, and a pharmaceutically acceptable carrier.

In more detailed embodiments, the disclosure relates to the following technical solutions:

1. A compound of Formula (X):

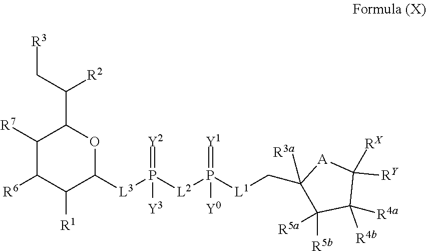

or a pharmaceutically acceptable salt, a stereoisomer, stable isotope, prodrug, or a tautomer thereof, wherein:
R^{X} is:
(A) a moiety having formula (X-Ia), (X-Ib), or (X-Ic):

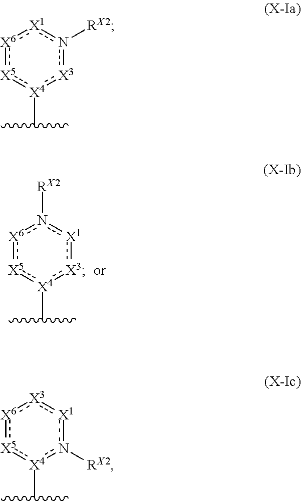

wherein:
X¹ is selected from the group consisting of: C(═O), C—OH, C═S, C—SH, C—NH₂, and C(═NH);
X³, X⁵, and X⁶ are each independently selected from the group consisting of: N, NH, N(R^{Xn}), CH, CR^{Xc}, C(═O), C(═S), C(═NH), and C(═NR^{Xn});
X⁴ is N or C;
R^{X2} is —H, RAN, or is absent when a double bond is present between NR^{X2} and an adjacent ring atom; and
each occurrence of [symbol] is independently a single bond or a double bond;
   provided that formulas (X-Ia), (X-Ib), and (X-Ic) each include from 1-2 endocyclic double bonds;
   provided that when X⁴ is C, then a double bond is present between X⁴ and an adjacent ring atom; and
   provided that when formulas (X-Ia), (X-Ib), and (X-Ic) each include only 1 endocyclic double bond, then X⁴ is N and/or one or more of X³, X⁵, and X⁶ are each independently selected from the group consisting of: N, NH, N(R^{Xn}), C(═O), C(═S), C(═NH), and C(═NR^{Xn});
(B) pyridinyl, pyrmidinyl, pyrazinyl, pyridazinyl, or triazinyl each of which is optionally substituted with from 1-3 R^{Xc}, provided that any R^{Xc} group that is ortho or para to a ring nitrogen of (B) is other than —OH, —SH, or NH₂;
(C) a moiety having formula (X-II):

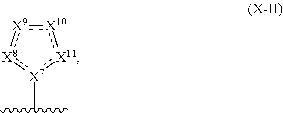

wherein:
X⁷ is C or N;
X⁸, X⁹, X¹⁰, and X¹¹ are each independently selected from the group consisting of: CH, C(R^{Xc}), N, N(H), N(R^{Xn}), O, S, C(═O), C(═S), C(═NH), and C(═NR^{Xn}); and
each [symbol] is independently a single bond or a double bond, provided that from 1-4 of X⁷—X¹¹ is independently selected from group consisting of C, CH, C(R^{Xc}), C(═O), C(═S), C(═NH), and C(═NR^{Xn}), and (X-II) is aromatic;
(D) C₆₋₁₀ aryl optionally substituted with from 1-4 R^{Xc}; or
(E) bicyclic heteroaryl having 8-12 ring atoms, wherein from 1-5 ring atoms are heteroatoms each independently selected from the group consisting of: N, N(H), N(R^{Xn}), O, and S(—O)₀₋₂, and wherein one or more ring carbon atoms of the heteroaryl is optionally substituted with from 1-4 substituents each independently selected from the group consisting of oxo and R^{Xc}.

each occurrence of R^{Xc} is independently selected from the group consisting of: R^{c}, R^{b}, and -(L^{b})_{b}-R^{b};
each occurrence of R^{Xn} is independently selected from the group consisting of: R^{d}, R^{b}, and -(L^{b})_{b}-R^{b};
R^{Y}, R^{a}, R^{4b}, R^{5a}, and R^{5b} are each independently selected from the group consisting of:
   H, —OH, —SH, -halo, cyano, or azido;
   C₁₋₆ alkyl, C₁₋₆ haloalkyl, C₂₋₆ alkenyl, C₂₋₆ haloalkenyl, C₂₋₆ alkynyl, or C₂₋₆ haloalkynyl, each of which is optionally substituted with from 1-6 R^{a};
   C₁₋₄ alkoxy or C₁₋₄ thioalkoxy, each of which is optionally substituted with from 1-6 R^{a};
   —OR⁹, —NR^{e}R^{f};
   —R^{b} or -(L^{b})_{b}-R^{b};
   —OP(═O)(OR′)(OR″); and
   —OC(═O) (C₁₋₆ alkyl) optionally substituted with from 1-6 R^{a}; or
L¹, L², L³ and A are each independently selected from the group consisting of: —O—, —S—, —NR^{L1}, and —C(R^{L2}) (R^{L2})—;
Y¹ and Y² are each independently selected from the group consisting of: O and S; Y⁰ and Y³ are each independently selected from the group consisting of: —OH, —OR⁹, —SH, and —SR′,
R¹, R², R⁶, and R⁷ are each independently selected from the group consisting of: H, D, -halo, OH, —SH, cyano, —OR⁹, —OC(═O)R⁹, —NR^{e}R^{f}, —NR^{e}C(═O)R⁹, —OP(═O)(OR′)(OR″), —OS(═O)₁₋₂R⁹, C₁₋₆ alkyl, C₁₋₆ haloalkyl, and —OR⁸;
R³ is selected from the group consisting of: H, D, -halo, —OH, —SH, cyano, —C(═O)OH, —C(═O)O(C₁₋₄ alkyl), —C(═O)NR′R″, —OR¹⁰, —OC(═O)R¹⁰, —NR^{e}R^{f}, —NR^{e}C(═O)R¹⁰, —OP(═O)(OR′)(OR″), —OS(═O)₁₋₂R¹⁰, C₁₋₆ alkyl, C₁₋₆ haloalkyl, and —OR⁸;
R^{3a} is selected from the group consisting of: —OH, —SH, —H, -halo, cyano, C₁₋₆ alkyl, C₁₋₆ haloalkyl, —C(═O)OH, —C(═O)O(C₁₋₄ alkyl), —C(═O)NR′R″, —OP(═O)(OR′)(OR″), C₁₋₄ alkoxy, C₁₋₄ haloalkoxy, —OR⁸, and —NR^{e}R^{f};
each occurrence of R⁸ is independently selected from the group consisting of:
   —C(═O) C₁₋₂₀ alkyl optionally substituted with from 1-10 substituents independently selected from the group consisting of: R^{a}, R^{b}, and -(L^{b})_{b}-R^{b};
   —C(═O)—(R^{b2})ₘ₁—R^{8b}, wherein each R^{b2} is independently a divalent R^{b} group, m1 is an integer from 1 to 6, and R^{8b} is —H or R^{c};

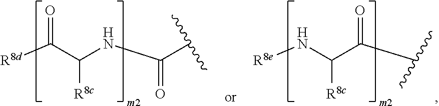

wherein:
0 m2 is an integer from 1 to 10;
each R^{8c} is independently selected from the group consisting of: —H; C₁₋₆ alkyl, which is optionally substituted with from 1-4 R^{a}; —R^{b}; and —(C₁₋₆ alkylene)-R^{b};
R^{8d} is selected from the group consisting of: —H, —OH, —C₁₋₄ alkoxy, and NR^{e}R^{f}; and
R^{8e} is selected from the group consisting of: —H, C₁₋₄ alkyl, C(═O) C₁₋₄ alkyl, and C(═O)OC₁₋₄ alkyl;
each occurrence of R⁹ is independently selected from the group consisting of: C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl, 3- to 7-membered heterocyclyl, C₆₋₁₀ aryl, or 5- to 10-membered heteroaryl;
each occurrence of R¹⁰ is independently selected from the group consisting of: C₁₋₂₀ alkyl, C₁₋₂₀ haloalkyl, C₂₋₂₀ alkenyl, C₂₋₂₀ haloalkenyl, C₂₋₂₀ alkynyl, C₂₋₂₀ haloalkynyl, C₃₋₇ cycloalkyl, 3- to 7-membered heterocyclyl, C₆₋₁₀ aryl, or 5- to 10-membered heteroaryl;
each occurrence of R^{L1} is independently selected from the group consisting of: —H; C₁₋₆ alkyl, C₁₋₆ haloalkyl, C₂₋₆ alkenyl, C₂₋₆ haloalkenyl, C₂₋₆ alkynyl, or C₂₋₆ haloalkynyl, which is optionally substituted with 1-3 substituents each independently selected from the group consisting of NR′R″, —OH, C₁₋₄ alkoxy, and C₁₋₄ haloalkoxy; and —C(═O)R′;
each occurrence of R^{L2} is independently selected from the group consisting of: —H; -halo; —OH; —OR⁹; C₁₋₆ alkyl, C₁₋₆ haloalkyl, C₂₋₆ alkenyl, C₂₋₆ haloalkenyl, C₂₋₆ alkynyl, or C₂₋₆ haloalkynyl, which is optionally substituted with 1-3 substituents each independently selected from the group consisting of NR′R″, —OH, C₁₋₄ alkoxy, and C₁₋₄ haloalkoxy; and —C(═O)R′;
each occurrence of R^{a} is independently selected from the group consisting of: —H, —OH, -halo, —NR^{e}R^{f}, C₁₋₄ alkoxy, C₁₋₄ haloalkoxy, —C(═O)O(C₁₋₄ alkyl), —C(═O) (C₁₋₄ alkyl), —C(═O)OH, —C(═O)NR′R″, —S(═O)₁₋₂NR′R″, —S(—O)₁₋₂ (C₁₋₄ alkyl), and cyano;
each occurrence of R^{b} is independently selected from the group consisting of:
C₃₋₁₀ cycloalkyl or C₃₋₁₀ cycloalkenyl, each of which is optionally substituted with 1-4 R^{c};
heterocyclyl or heterocycloalkenyl of 3-10 ring atoms, wherein 1-3 ring atoms are heteroatoms, each independently selected from the group consisting of N, N(H), N(R^{d}), O, and S(═O)₀₋₂, and wherein the heterocyclyl or heterocycloalkenyl is optionally substituted with 1-4 R^{c};
heteroaryl of 5-10 ring atoms, wherein 1-3 ring atoms are heteroatoms, each independently selected from the group consisting of N, N(H), N(R^{d}), O, and S(═O)₀₋₂, and wherein the heteroaryl is optionally substituted with 1-4 R^{e}; and
C₆₋₁₀ aryl optionally substituted with 1-4 R^{e};
each occurrence of L^{b} is independently selected from the group consisting of: —O—, —NH—, —NR^{d}, —S(═O)₀₋₂, C(═O), and C₁₋₃ alkylene optionally substituted with 1-3 R^{a};
each occurrence of b is independently 1, 2, 3, or 4;
each occurrence of R^{e} is independently selected from the group consisting of: halo; cyano; C₁₋₁₀ alkyl, which is optionally substituted with 1-6 independently selected R^{a}; C₂₋₆ alkenyl; C₂₋₆ alkynyl; C₁₋₄ alkoxy; C₁₋₄ haloalkoxy; —S(═O)₁₋₂ (C₁₋₄ alkyl); —NR^{e}R^{f}; —OH; —SH; —S(═O)₁₋₂NR′R″; —C₁₋₄ thioalkoxy; —NO₂; —OC(═O) (C₁₋₄ alkyl); —OC(═O) H; —C(═O) (C₁₋₄ alkyl); —C(═O) H; —C(═O)O(C₁₋₄ alkyl); —C(═O)OH; and —C(═O)NR′R″;
each occurrence of R^{d} is independently selected from the group consisting of: C₁₋₆ alkyl optionally substituted with 1-3 independently selected R^{a}; —C(═O) (C₁₋₄ alkyl); —C(═O)O(C₁₋₄ alkyl); —C(═O)NR′R″; —S(═O)₁₋₂NR′R″; —S(═O)₁₋₂ (C₁₋₄ alkyl); —OH; and C₁₋₄ alkoxy;
each occurrence of R^{e} and R^{f} is independently selected from the group consisting of: —H; C₁₋₆ alkyl or C₁₋₆ haloalkyl, which is optionally substituted with 1-3 substituents each independently selected from the group consisting of NR′R″, —OH, halo, C₁₋₄ alkoxy, and C₁₋₄ haloalkoxy; —C(═O)R′; —C(═O)OR′; —C(═O)NR′R″; C(═NR″)NR′R″; —C(═O) C(═O)R′; —S(═O)₁₋₂NR′R″; —S(═O)₁₋₂R′; —OH; and C₁₋₄ alkoxy; or
R^{e}, and R^{f} taken together with the N atom connecting them form a saturated or unsaturated 3- to 7-membered heterocyclyl; and
each occurrence of R′ and R″ is independently selected from the group consisting of: —H; C₁₋₄ alkyl or C₁₋₄ haloalkyl, which is optionally substituted with 1-3 substituents each independently selected from the group consisting of: halo, cyano, C₁₋₄ alkoxy, C₁₋₄ haloalkoxy, and —OH;
provided that at least one of the following is true:
a) R^{4a} is selected from the group consisting of: C₂₋₆ alkenyl, C₂₋₆ haloalkenyl, C₂₋₆ alkynyl, and C₂₋₆ haloalkynyl;
b) R^{4b} is NR^{e}R^{f}.

2. The compound of technical solution 1, wherein R^{X} is bicyclic heteroaryl having 8-12 ring atoms, wherein from 1-5 ring atoms are heteroatoms each independently selected from the group consisting of: N, N(H), N(R^{Xn}), O, and S(═O)₀₋₂, and wherein one or more ring carbon atoms of the heteroaryl is optionally substituted with from 1-4 substituents each independently selected from the group consisting of oxo and R^{Xc}.

3. The compound of any one of technical solutions 1-2, wherein R^{X} is bicyclic heteroaryl having 9-10 (e.g., 9) ring atoms, wherein from 1-5 ring atoms are heteroatoms each independently selected from the group consisting of: N, N(H), N(R^{Xn}), O, and S(═O)₀₋₂, and wherein one or more ring carbon atoms of the heteroaryl is optionally substituted with from 1-4 substituents each independently selected from the group consisting of oxo and R^{Xc}.

4. The compound of any one of technical solutions 1-3, wherein R^{X} is

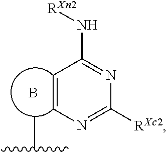

wherein: Ring B is heteroaryl having 5 ring atoms wherein from 1-3 ring atoms are heteroatoms each independently selected from the group consisting of: N, N(H), N(R^{X1}), O, and S, and wherein Ring B is optionally substituted with R^{Xc}; R^{Xn2} is —H or R^{Xn} (e.g., —H); and R^{Xc2} is —H or R^{Xc} (e.g., —H).

5. The compound of any one of technical solutions 1-4, wherein R^{Xn2} is —H; preferably, R^{Xc2} is —H.

6. The compound of any one of technical solutions 1-5, wherein R^{X} is selected from the group consisting of:

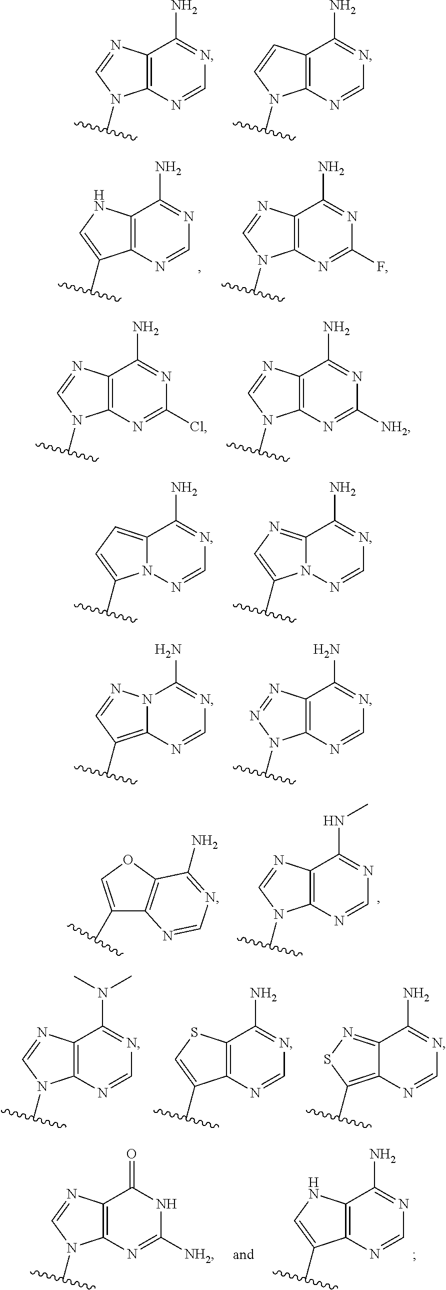

preferably

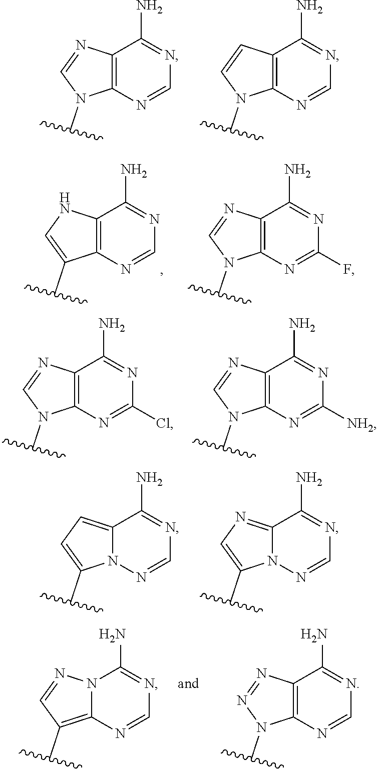

7. The compound of any one of technical solutions 1-6, wherein R^{Y} is H.

8. The compound of any one of technical solutions 1-7, wherein L¹ is —O—.

9. The compound of any one of technical solutions 1-8, wherein L² is —O—.

10. The compound of any one of technical solutions 1-9, wherein L³ is —O—.

11. The compound of any one of technical solutions 1-10, wherein Y⁰ is —SH.

12. The compound of any one of technical solutions 1-11, wherein R¹ is selected from the group consisting of: —OH, -halo (e.g., —F), —OP(═O)(OR′)(OR″), and —OR⁸; preferably is —OR⁸.

13. The compound of any one of technical solutions 1-12, wherein R¹ is —OH.

14. The compound of any one of technical solutions 1-13, wherein R⁶ and R⁷ are independently selected from the group consisting of: —OH, —SH, -halo (e.g., —F), —NR^{e}R^{f} (e.g., NH₂), —OP(═O)(OR′)(OR″), and —OR⁸; preferably is —OR⁸.

15. The compound of any one of technical solutions 1-14, wherein R⁶ and R⁷ are each —OH

16. The compound of any one of technical solutions 1-15, wherein R² is —OH, -halo (e.g., —F), —OP(═O)(OR′)(OR″), —OR⁸ or NR^{e}R^{f}; preferably is —OR⁸.

17. The compound of any one of technical solutions 1-16, wherein R² is —OH.

18. The compound of any one of technical solutions 1-17, wherein the carbon to which R² is attached has(S)-stereochemical configuration.

19. The compound of any one of technical solutions 1-18, wherein R³ is selected from the group consisting of: —OH, halo (e.g., —F), —OP(═O)(OR′)(OR″) (e.g., —OP(═O)(OH) 2), C(═O)OH, NR^{e}R^{f} (e.g., NH₂), —C(═O)NR′R″, and —OR⁸ (e.g., —OC(═O) (C₁₋₄ alkyl).

20. The compound of any one of technical solutions 1-19, wherein R³ is —OH or —OR⁸; preferably is —OR⁸.

21. The compound of any one of technical solutions 1-20, wherein R³ is —OH.

22. The compound of any one of technical solutions 1-21, wherein the moiety

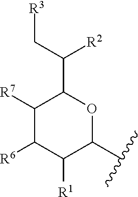

is selected from the group consisting of:

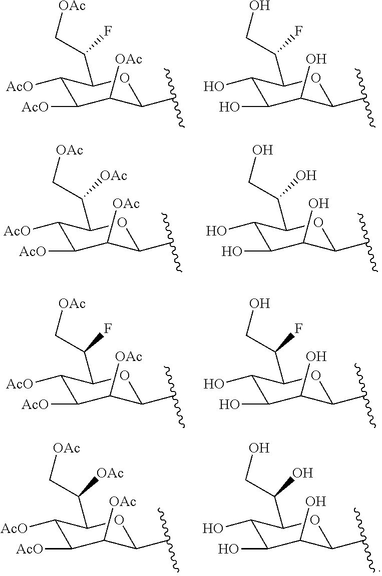

23. The compound of any one of technical solutions 1-22, wherein Y¹ and Y² are O.

24. The compound of any one of technical solutions 1-23, wherein Y³ is —OH.

25. The compound of any one of technical solutions 1-24, wherein R^{4a} is selected from the group consisting of: C₂₋₆ alkenyl, C₂₋₆ haloalkenyl, C₂₋₆ alkynyl, and C₂₋₆ haloalkynyl; R^{4b} is selected from the group consisting of: —OH, —OR⁹, and -halo.
26. The compound of any one of technical solutions 1-25, wherein the moiety

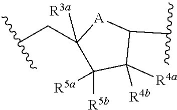

is selected from the group consisting of:

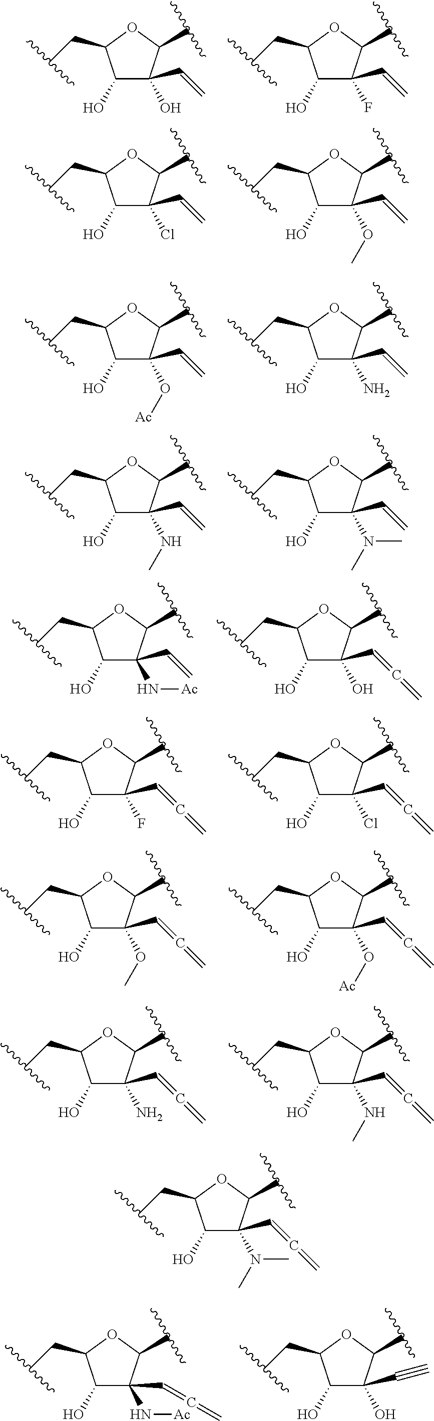

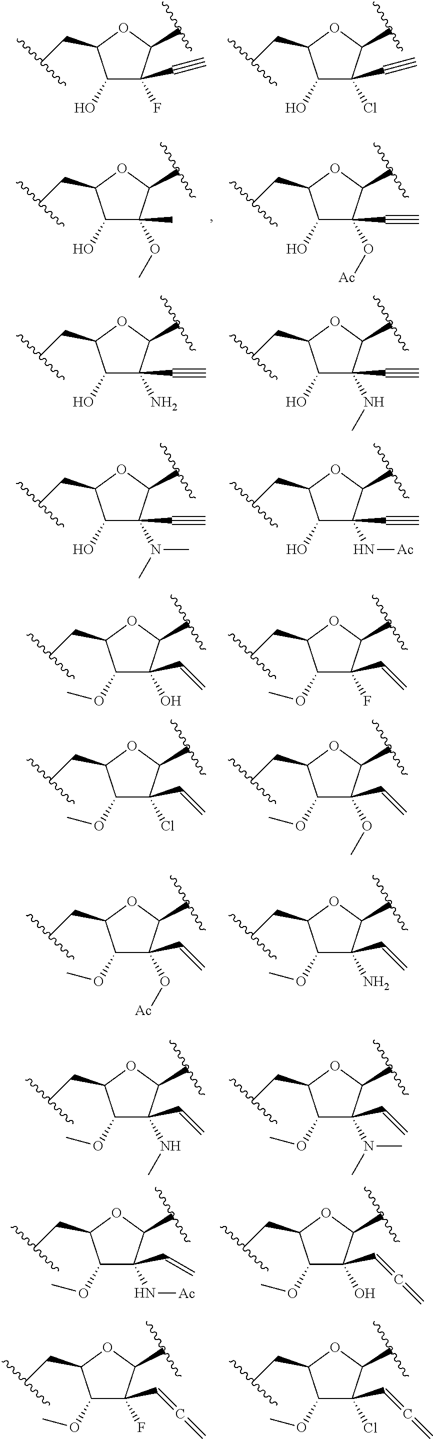

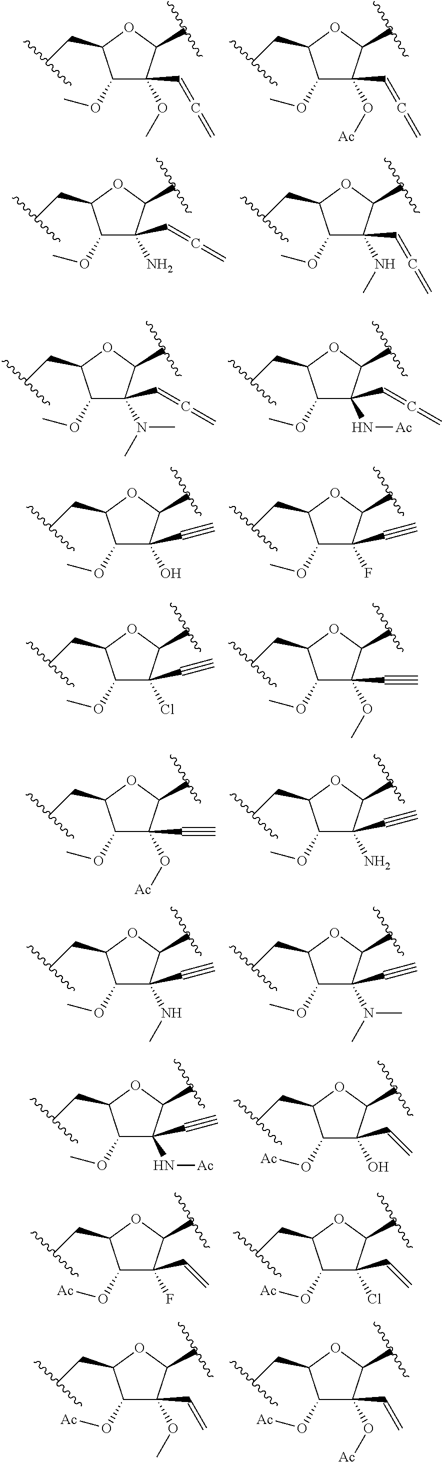

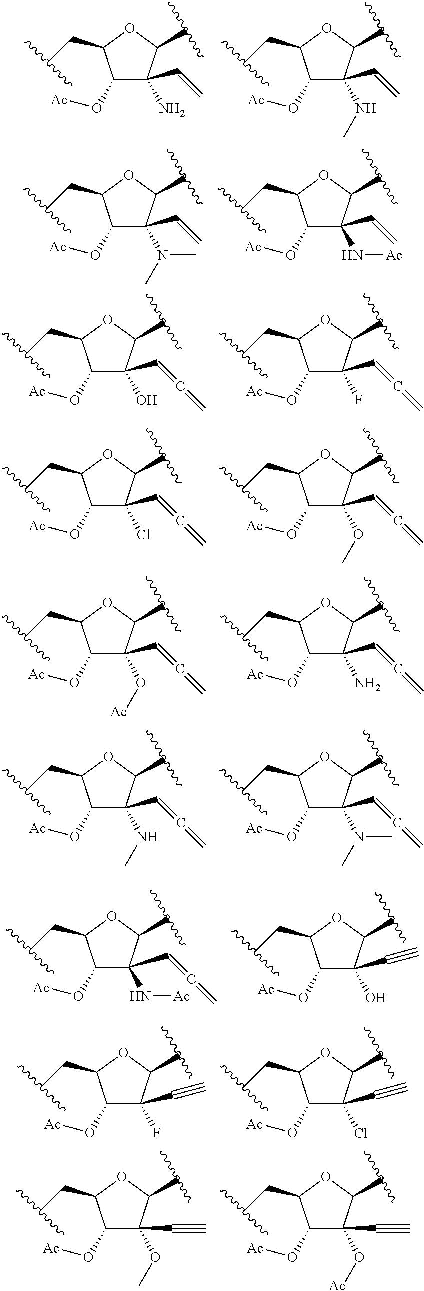

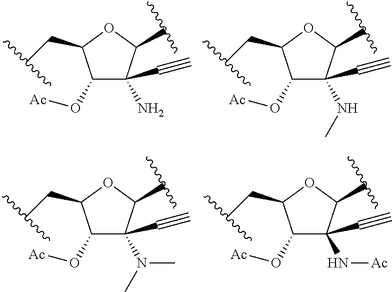

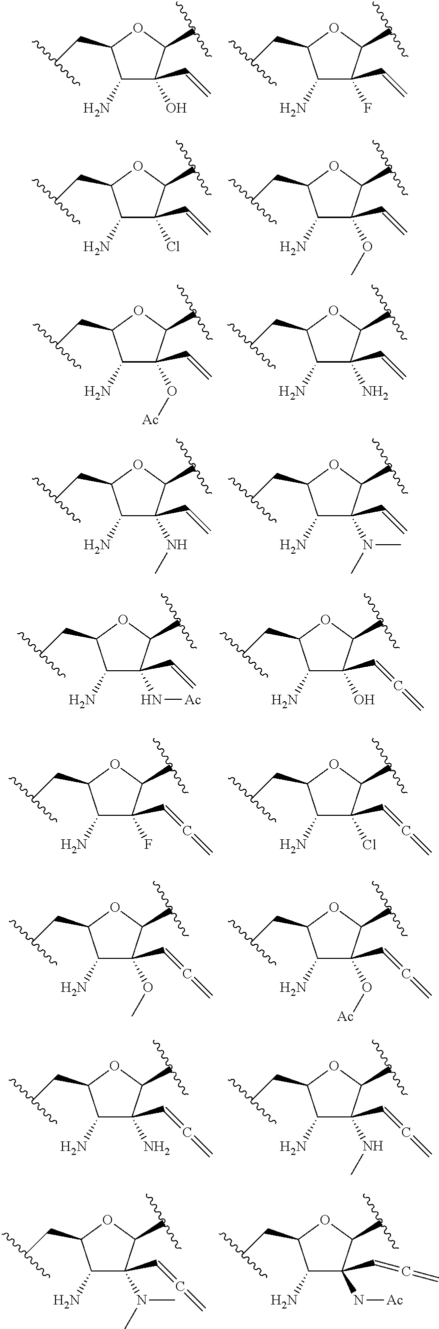

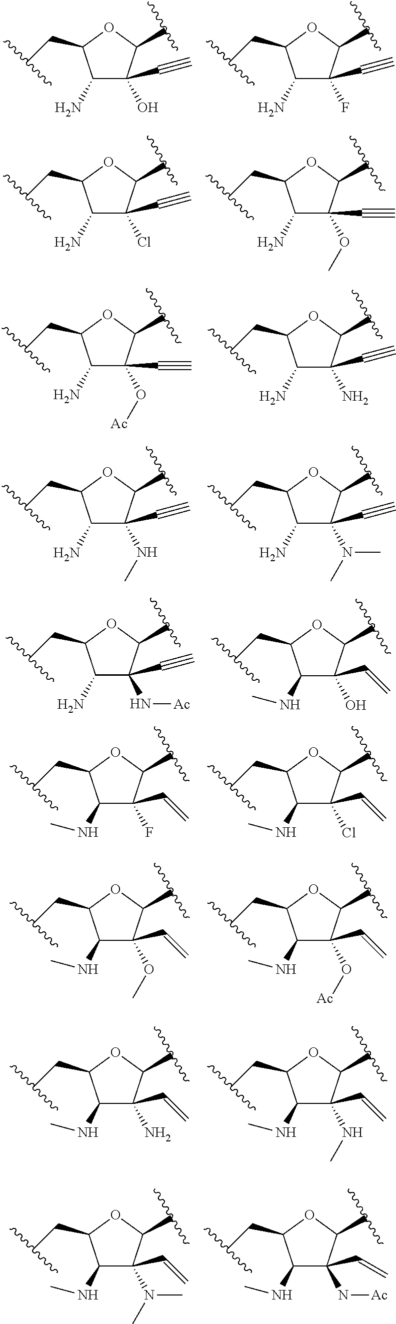

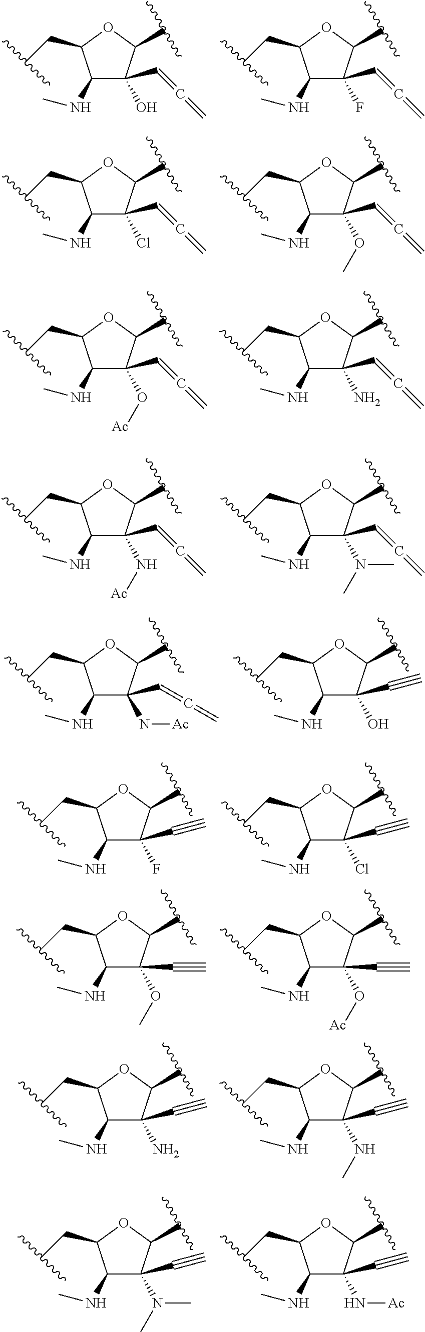

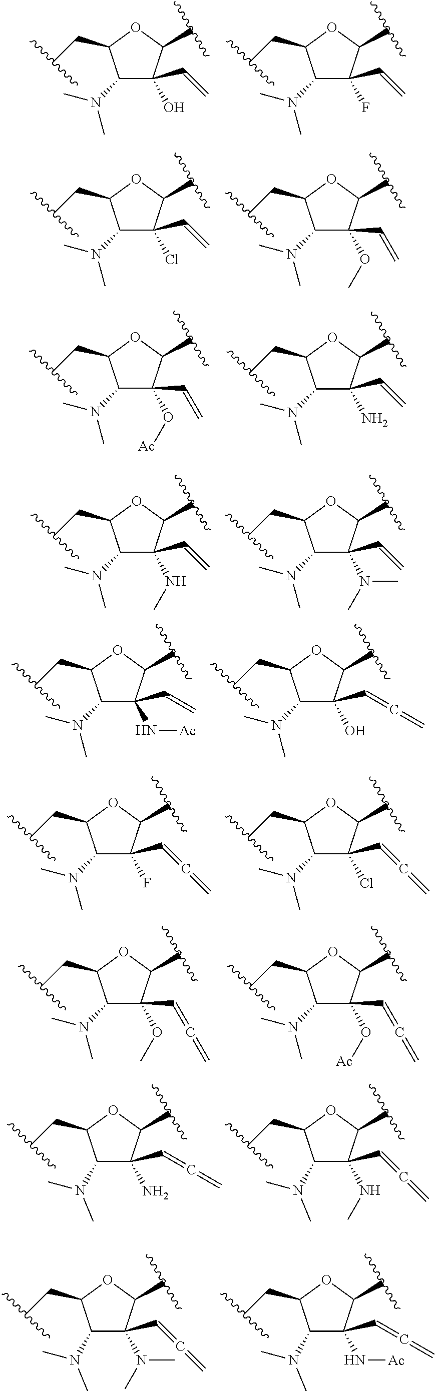

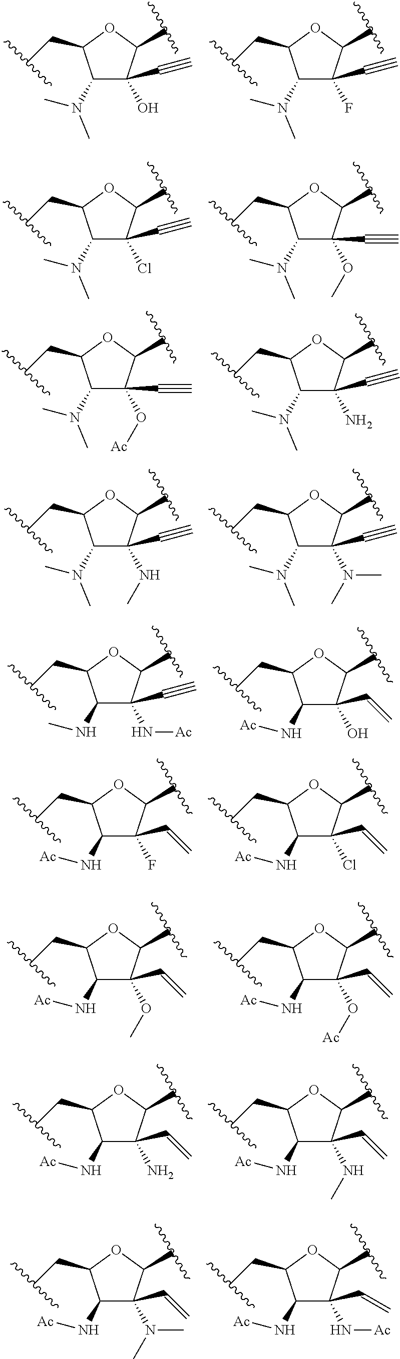

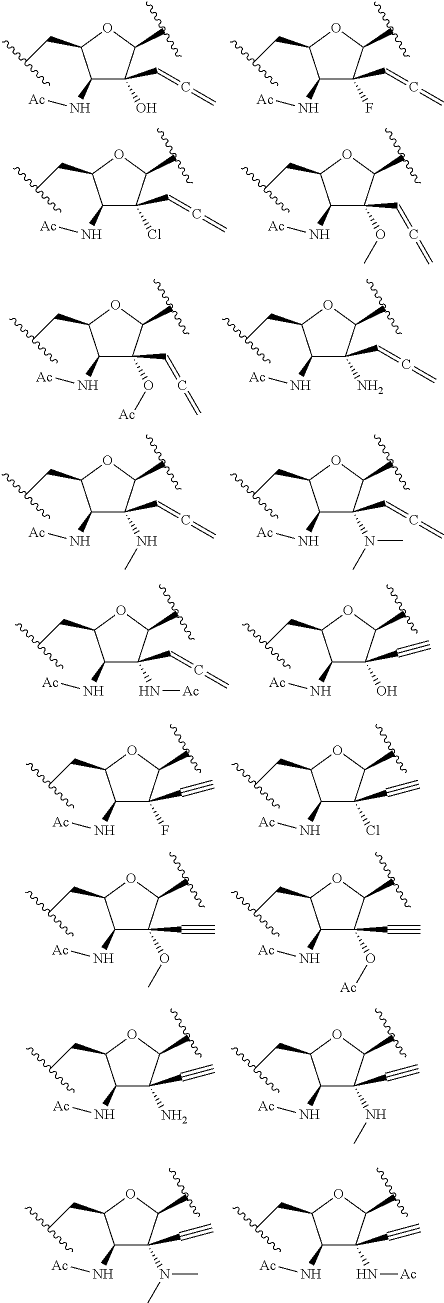

27. The compound of any one of technical solutions 1-26, wherein the moiety

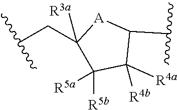

is selected from the group consisting of:

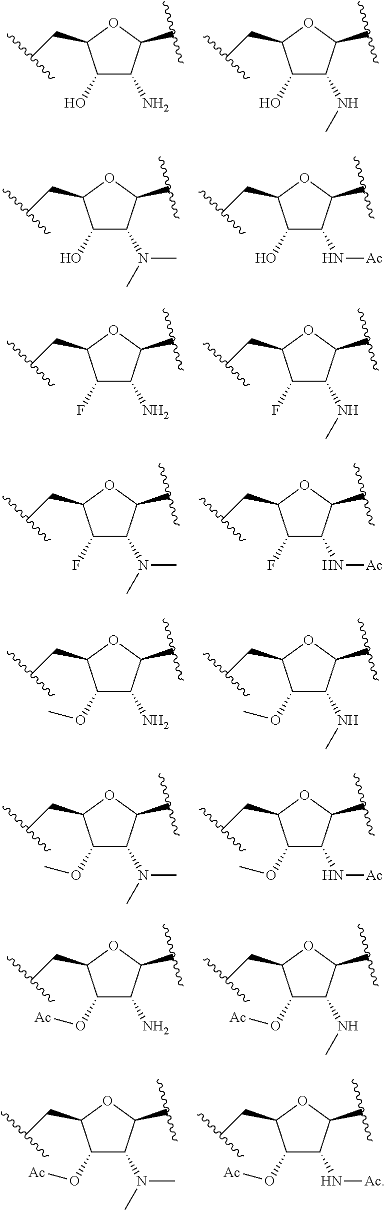

28. A compound of Formula (I-h), (I-h-1), (I-h-2), (I-h-3), (I-h-4) or (I-h-5):

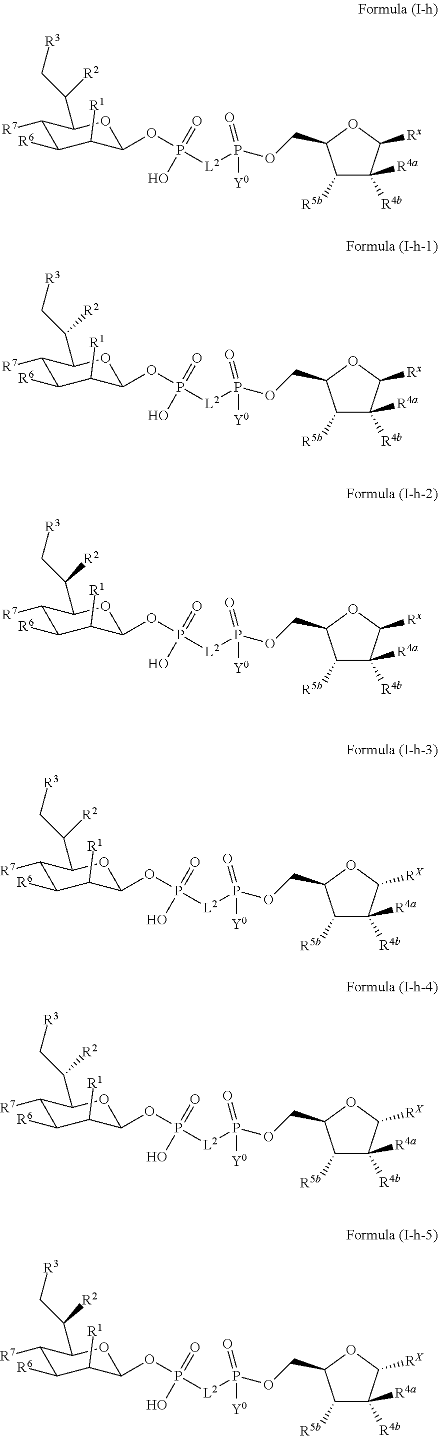

or a pharmaceutically acceptable salt, a stereoisomer, a stable isotope, a prodrug, or a tautomer thereof, wherein:
R^{X} is:
(A) a moiety having formula (X-la), (X-Ib), or (X-Ic):

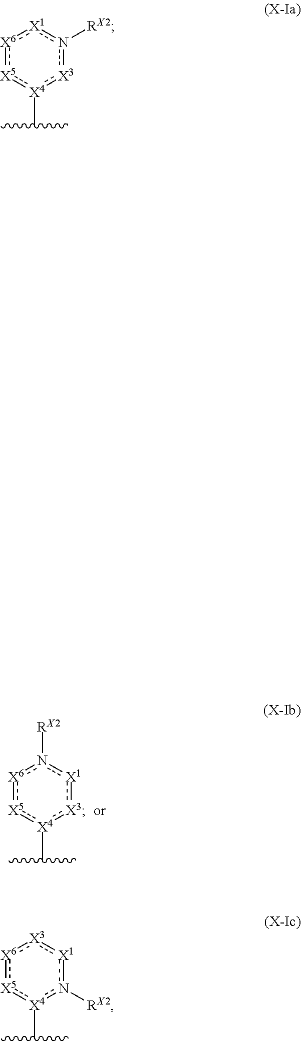

wherein:
X¹ is selected from the group consisting of: C(═O), C—OH, C═S, C—SH, C—NH₂, and C(═NH);
X³, X⁵, and X⁶ are each independently selected from the group consisting of: N, NH, N(R^{Xn}), CH, CR^{Xc}, C(═O), C(═S), C(═NH), and C(═NR^{Xn});
X⁴ is N or C;
R^{X2} is —H, R^{Xn}, or is absent when a double bond is present between NR^{X2} and an adjacent ring atom; and
each occurrence of [symbol] is independently a single bond or a double bond;
   provided that formulas (X-Ia), (X-Ib), and (X-Ic) each include from 1-2 endocyclic double bonds;
   provided that when X⁴ is C, then a double bond is present between X⁴ and an adjacent ring atom; and
   provided that when formulas (X-Ia), (X-Ib), and (X-Ic) each include only 1 endocyclic double bond, then X⁴ is N and/or one or more of X³, X⁵, and X⁶ are each independently selected from the group consisting of: N, NH, N(R^{Xn}), C(═O), C(═S), C(═NH), and C(═NR^{Xn});
   (B) pyridinyl, pyrmidinyl, pyrazinyl, pyridazinyl, or triazinyl each of which is optionally substituted with from 1-3 R^{Xc}, provided that any R^{Xc} group that is ortho or para to a ring nitrogen of (B) is other than —OH, —SH, or NH₂;
   (C) a moiety having formula (X-II):

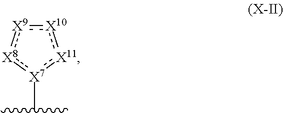

wherein:
X⁷ is C or N;
X⁸, X⁹, X¹⁰, and X¹¹ are each independently selected from the group consisting of: CH, C(R^{Xc}), N, N(H), N(R^{Xn}), O, S, C(═O), C(═S), C(═NH), and C(═NR^{Xn}); and
each [symbol] is independently a single bond or a double bond,
provided that from 1-4 of X⁷—X¹¹ is independently selected from group consisting of C, CH, C(R^{Xc}), C(═O), C(═S), C(═NH), and C(═NR^{Xn}), and (X-II) is aromatic;
   (D) C₆₋₁₀ aryl optionally substituted with from 1-4 R^{Xc}; or
   (E) bicyclic heteroaryl having 8-12 ring atoms, wherein from 1-5 ring atoms are heteroatoms each independently selected from the group consisting of: N, N(H), N(R^{Xn}), O, and S(—O)₀₋₂, and wherein one or more ring carbon atoms of the heteroaryl is optionally substituted with from 1-4 substituents each independently selected from the group consisting of oxo and R^{Xc};
each occurrence of R^{Xc} is independently selected from the group consisting of: R^{c}, R^{b}, and -(L^{b})_{b}-R^{b};
each occurrence of R^{Xn} is independently selected from the group consisting of: R^{d}, R^{b}, and -(L^{b})_{b}-R^{b};
R^{4a} is selected from the group consisting of: C₂₋₆ alkenyl, C₂₋₆ haloalkenyl, C₂₋₆ alkynyl, and C₂₋₆ haloalkynyl;
R^{4b} and R^{5b} are each independently selected from the group consisting of:
   H, —OH, —SH, -halo, cyano, or azido;
   C₁₋₆ alkyl, C₁₋₆ haloalkyl, C₂₋₆ alkenyl, C₂₋₆ haloalkenyl, C₂₋₆ alkynyl, or C₂₋₆ haloalkynyl, each of which is optionally substituted with from 1-6 R^{a};
   C₁₋₄ alkoxy or C₁₋₄ thioalkoxy, each of which is optionally substituted with from 1-6 R^{a};
   —OR⁹, —NR^{e}R^{f};
   —R^{b} or -(L^{b})_{b}-R^{b};
   —OP(═O)(OR′)(OR″); and
   —OC(═O) (C₁₋₆ alkyl) optionally substituted with from 1-6 R^{a}; or
L² is selected from the group consisting of: —O—, —S—, —NR^{L1}—, and —C(R^{L2}) (R^{L2})—;
Y⁰ is selected from the group consisting of: —OH and —SH;
R³ is selected from the group consisting of: H, D, -halo, —OH, —SH, cyano, —OR¹⁰, —OC(═O)R¹⁰, —NR^{e}R^{f}, —NR^{e}C(═O)R¹⁰, —OP(═O)(OR′)(OR″), —OS(—O)₁₋₂R¹⁰, C₁₋₆ alkyl, and C₁₋₆ haloalkyl;
R¹, R², R⁶, and R⁷ are each independently selected from the group consisting of: H, D, -halo, —OH, —SH, cyano, —OR⁹, —OC(═O)R⁹, —NR^{e}R^{f}, —NR^{e}C(═O)R⁹, —OP(═O)(OR′)(OR″), —OS(═O)₁₋₂R⁹, C₁₋₆ alkyl, and C₁₋₆ haloalkyl;
each occurrence of R⁹ is independently selected from the group consisting of: C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl, 3- to 7-membered heterocyclyl, C₆₋₁₀ aryl, and 5- to 10-membered heteroaryl;
each occurrence of R¹⁰ is independently selected from the group consisting of: C₁₋₂₀ alkyl, C₁₋₂₀ haloalkyl, C₂₋₂₀ alkenyl, C₂₋₂₀ haloalkenyl, C₂₋₂₀ alkynyl, C₂₋₂₀ haloalkynyl, C₃₋₇ cycloalkyl, 3- to 7-membered heterocyclyl, C₆₋₁₀ aryl, and 5- to 10-membered heteroaryl;
each occurrence of R^{L1} is independently selected from the group consisting of: —H; C₁₋₆ alkyl, C₁₋₆ haloalkyl, C₂₋₆ alkenyl, C₂₋₆ haloalkenyl, C₂₋₆ alkynyl, or C₂₋₆ haloalkynyl, which is optionally substituted with 1-3 substituents each independently selected from the group consisting of NR′R″, —OH, C₁₋₄ alkoxy, and C₁₋₄ haloalkoxy; and —C(═O)R′;
each occurrence of R^{L2} is independently selected from the group consisting of: —H; -halo; —OH; —OR⁹; C₁₋₆ alkyl, C₁₋₆ haloalkyl, C₂₋₆ alkenyl, C₂₋₆ haloalkenyl, C₂₋₆ alkynyl, or C₂₋₆ haloalkynyl, which is optionally substituted with 1-3 substituents each independently selected from the group consisting of NR′R″, —OH, C₁₋₄ alkoxy, and C₁₋₄ haloalkoxy; and —C(═O)R′;
each occurrence of R^{a} is independently selected from the group consisting of: —H, —OH, -halo, —NR^{e}R^{f}, C₁₋₄ alkoxy, C₁₋₄ haloalkoxy, —C(═O)O(C₁₋₄ alkyl), —C(═O) (C₁₋₄ alkyl), —C(═O)OH, —C(═O)NR′R″, —S(═O)₁₋₂NR′R″, —S(—O)₁₋₂ (C₁₋₄ alkyl), and cyano;
each occurrence of R^{b} is independently selected from the group consisting of:
C₃₋₁₀ cycloalkyl or C₃₋₁₀ cycloalkenyl, each of which is optionally substituted with 1-4 R^{c};
heterocyclyl or heterocycloalkenyl of 3-10 ring atoms, wherein 1-3 ring atoms are heteroatoms, each independently selected from the group consisting of N, N(H), N(R^{d}), O, and S(—O)₀₋₂, and wherein the heterocyclyl or heterocycloalkenyl is optionally substituted with 1-4 R^{c};
heteroaryl of 5-10 ring atoms, wherein 1-3 ring atoms are heteroatoms, each independently selected from the group consisting of N, N(H), N(R^{d}), O, and S(═O)₀₋₂, and wherein the heteroaryl is optionally substituted with 1-4 R^{c}; and
C₆₋₁₀ aryl optionally substituted with 1-4 R^{e};
each occurrence of L^{b} is independently selected from the group consisting of: —O—, —NH—, —NR^{d}, —S(═O)₀₋₂, C(═O), and C₁₋₃ alkylene optionally substituted with 1-3 R^{a};
each occurrence of b is independently 1, 2, 3, or 4;
each occurrence of R^{e} is independently selected from the group consisting of: halo;
cyano; C₁₋₁₀ alkyl, which is optionally substituted with 1-6 independently selected R^{a}; C₂₋₆ alkenyl; C₂₋₆ alkynyl; C₁₋₄ alkoxy; C₁₋₄ haloalkoxy; —S(—O)₁₋₂ (C₁₋₄ alkyl); —NR^{e}R^{f}; OH; —SH; —S(═O)₁₋₂NR′R″; —C₁₋₄ thioalkoxy; —NO₂; —OC(═O) (C₁₋₄ alkyl); —OC(═O) H; —C(═O) (C₁₋₄ alkyl); —C(═O) H; —C(═O)O(C₁₋₄ alkyl); —C(═O)OH; and —C(═O)NR′R″;
each occurrence of R^{d} is independently selected from the group consisting of: C₁₋₆ alkyl optionally substituted with 1-3 independently selected R^{a}; —C(═O) (C₁₋₄ alkyl); —C(═O)O(C₁₋₄ alkyl); —C(═O)NR′R″; —S(═O)₁₋₂NR′R″; —S(═O)₁₋₂ (C₁₋₄ alkyl); —OH; and C₁₋₄ alkoxy;
each occurrence of R^{e} and R^{f} is independently selected from the group consisting of: —H; C₁₋₆ alkyl or C₁₋₆ haloalkyl, which is optionally substituted with 1-3 substituents each independently selected from the group consisting of NR′R″, —OH, halo, C₁₋₄ alkoxy, and C₁₋₄ haloalkoxy; —C(═O)R′; —C(═O)OR′; —C(═O)NR′R″; C(═NR″)NR′R″; —C(═O) C(═O)R′; —S(═O)₁₋₂NR′R″; —S(═O)₁₋₂R′; —OH; and C₁₋₄ alkoxy; and
each occurrence of R′ and R″ is independently selected from the group consisting of: —H; C₁₋₄ alkyl or C₁₋₄ haloalkyl, which is optionally substituted with 1-3 substituents each independently selected from the group consisting of: halo, cyano, C₁₋₄ alkoxy, C₁₋₄ haloalkoxy, and —OH.

29. The compound of technical solution 28, wherein:

R^{X} is selected from the group consisting of:

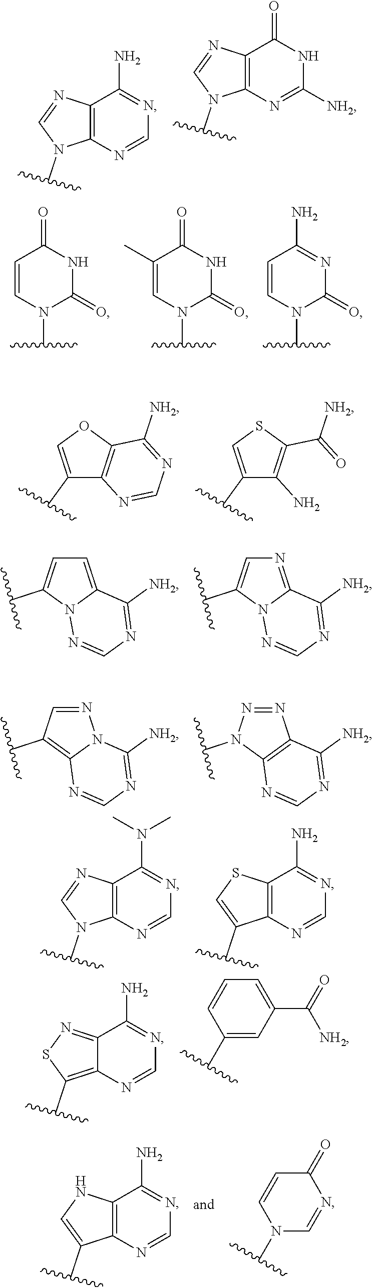

preferably,

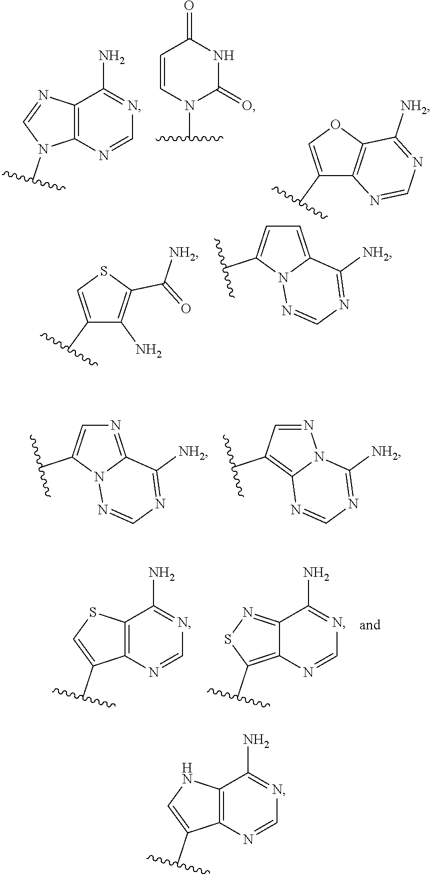

preferably,

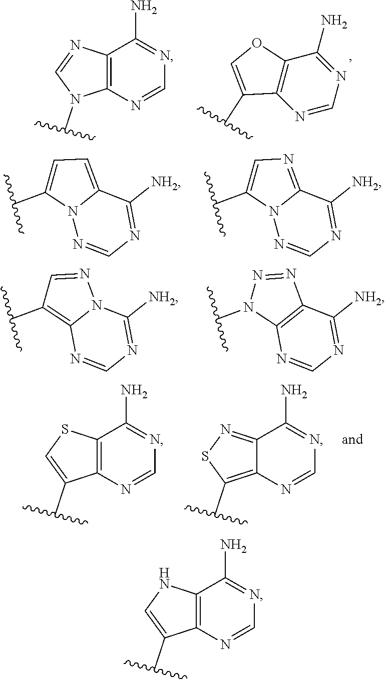

preferably,

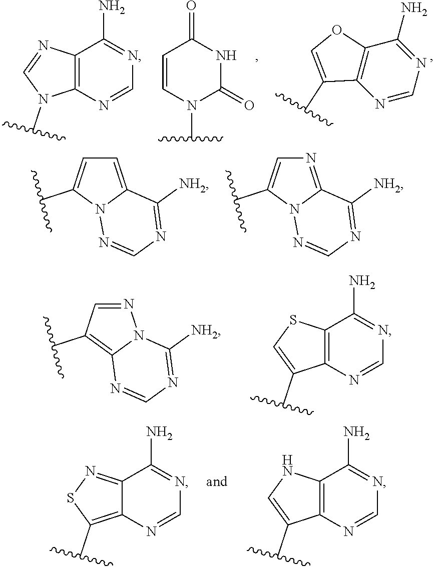

preferably,

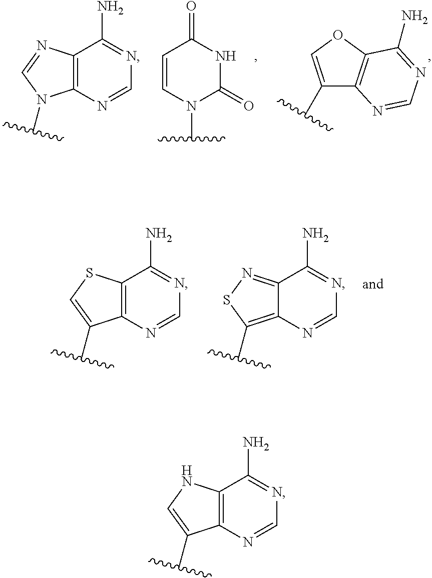

preferably,

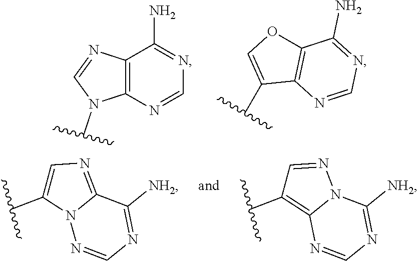

preferably,

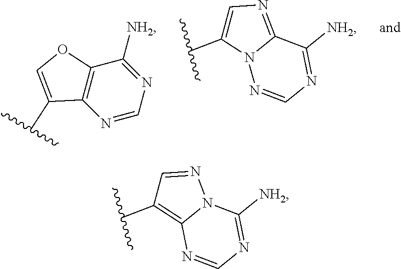 [SMILES: *c1coc2c(N)ncnc12 ; *c1cnc2c(N)ncnn12 ; *c1cnn2c(N)ncnc12]

preferably,

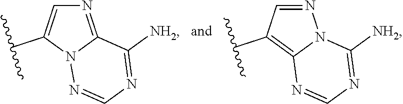

preferably,

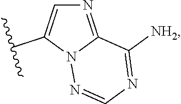 [SMILES: *c1cnc2c(N)ncnn12]

preferably,

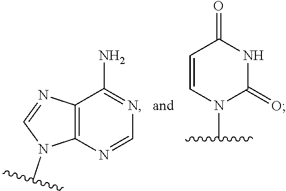

R^{4a} is selected from the group consisting of: C₂₋₆ alkenyl, C₂₋₆ haloalkenyl, C₂₋₆ alkynyl, and C₂₋₆ haloalkynyl, preferably, C₂₋₆ alkenyl, C₂₋₆ haloalkenyl, C₂₋₆ alkynyl, and C₂₋₆ haloalkynyl that containing one to three double bond or triple bond, preferably C₂₋₆ alkenyl, and C₂₋₆ haloalkenyl that containing cumulated double bonds, preferably C₂₋₆ alkenyl, and C₂₋₆ haloalkenyl that containing conjugate double bonds, preferably C₂₋₆ alkenyl, and C₂₋₆ haloalkenyl that containing independent double bonds, preferably, ethenyl, propenyl, ethynyl, and propynyl, preferably, ethenyl, and ethynyl;
R^{4b} is selected from the group consisting of: —H, —OH, —OR⁹, —OC(═O)R⁹, —NR^{e}R^{f}, and -halo, preferably, —F, —OH, —OR⁹, and —NR^{e}R^{f}, preferably, —F, —OH, —OMe, and —NH₂, preferably, —F, —OH, and —OMe;
R^{5b} is independently selected from the group consisting of:
   H, —OH, —SH, -halo, cyano, or azido;
   C₁₋₆ alkyl, C₁₋₆ haloalkyl, C₂₋₆ alkenyl, C₂₋₆ haloalkenyl, C₂₋₆ alkynyl, or C₂₋₆ haloalkynyl, each of which is optionally substituted with from 1-6 R^{a};
   C₁₋₄ alkoxy or C₁₋₄ thioalkoxy, each of which is optionally substituted with from 1-6 R^{a};
   —OR⁹, —NR^{e}R^{f};
   —OC(═O) (C₁₋₆ alkyl) optionally substituted with from 1-6 R^{a};
preferably, R^{5b} is —OH;
L² is selected from the group consisting of: —O—, —S—, —NR^{L1}—, and —C(R^{L2}) (R^{L2})—, preferably, —O—;
Y⁰ is selected from the group consisting of: OH and —SH, preferably, —SH;
R³ is selected from the group consisting of: H, D, -halo, —OH, —SH, cyano, —OR¹⁰, —OC(═O)R¹⁰, —NR^{e}R^{f}, —NR^{e}C(═O)R¹⁰, —OP(—O)(OR′)(OR″), —OS(═O)₁₋₂R¹⁰, C₁₋₆ alkyl, and C₁₋₆ haloalkyl;
R¹, R², R⁶, and R⁷ are each independently selected from the group consisting of: H, D, -halo, —OH, —SH, cyano, —OR⁹, —OC(═O)R⁹, —NR^{e}R^{f}, —NR^{e}C(═O)R⁹, —OP(═O)(OR′)(OR″), —OS(═O)₁₋₂R⁹, C₁₋₆ alkyl, and C₁₋₆ haloalkyl;
preferably, R² is -halo, —OH, or —OC(═O)R⁹, preferably, —OH, or —OC(═O)R⁹, preferably, —OH, or —OC(═O) C₁₋₆ alkyl, preferably, —OH;
preferably, R³ is selected from the group consisting of: —OH, —OR¹⁰, and —OC(═O)R¹⁰, preferably, —OH, or —OC(═O) C₁₋₂₀ alkyl, preferably, —OH;
preferably, R¹, R⁶, and R⁷ are each independently —OH, or —OC(═O)R⁹, preferably, —OH, or —OC(═O) C₁₋₆ alkyl, preferably, —OH;
each occurrence of R⁹ is independently selected from the group consisting of: C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl, 3- to 7-membered heterocyclyl, C₆₋₁₀ aryl, or 5- to 10-membered heteroaryl;
each occurrence of R¹⁰ is independently selected from the group consisting of: C₁₋₂₀ alkyl, C₁₋₂₀ haloalkyl, C₂₋₂₀ alkenyl, C₂₋₂₀ haloalkenyl, C₂₋₂₀ alkynyl, C₂₋₂₀ haloalkynyl, C₃₋₇ cycloalkyl, 3- to 7-membered heterocyclyl, C₆₋₁₀ aryl, or 5- to 10-membered heteroaryl;
each occurrence of R^{L1} is independently selected from the group consisting of: —H; C₁₋₆ alkyl, C₁₋₆ haloalkyl, C₂₋₆ alkenyl, C₂₋₆ haloalkenyl, C₂₋₆ alkynyl, or C₂₋₆ haloalkynyl, which is optionally substituted with 1-3 substituents each independently selected from the group consisting of NR′R″, —OH, C₁₋₄ alkoxy, and C₁₋₄ haloalkoxy; and —C(═O)R′;
each occurrence of R^{L2} is independently selected from the group consisting of: —H; -halo; —OH; —OR⁹; C₁₋₆ alkyl, C₁₋₆ haloalkyl, C₂₋₆ alkenyl, C₂₋₆ haloalkenyl, C₂₋₆ alkynyl, or C₂₋₆ haloalkynyl, which is optionally substituted with 1-3 substituents each independently selected from the group consisting of NR′R″, —OH, C₁₋₄ alkoxy, and C₁₋₄ haloalkoxy; and —C(═O)R′;
each occurrence of R^{a} is independently selected from the group consisting of: —H; —OH; -halo; —NR^{e}R^{f}; C₁₋₄ alkoxy; C₁₋₄ haloalkoxy; —C(═O)O(C₁₋₄ alkyl); —C(═O) (C₁₋₄ alkyl); —C(═O)OH; —C(═O)NR′R″; —S(═O)₁₋₂NR′R″; —S(═O)₁₋₂ (C₁₋₄ alkyl); and cyano;
each occurrence of R^{e} and R^{f} is independently selected from the group consisting of: —H; C₁₋₆ alkyl or C₁₋₆ haloalkyl, which is optionally substituted with 1-3 substituents each independently selected from the group consisting of NR′R″, —OH, halo, C₁₋₄ alkoxy, and C₁₋₄ haloalkoxy; —C(═O)R′; —C(═O)OR′; —C(═O)NR′R″; C(═NR″)NR′R″; —C(═O) C(═O)R′; —S(═O)₁₋₂NR′R″; —S(═O)₁₋₂R′; —OH; and C₁₋₄ alkoxy; and
each occurrence of R′ and R″ is independently selected from the group consisting of: —H; C₁₋₄ alkyl or C₁₋₄ haloalkyl, which is optionally substituted with 1-3 substituents each independently selected from the group consisting of: halo, cyano, C₁₋₄ alkoxy, C₁₋₄ haloalkoxy, and —OH.

30. The compound of technical solution 28, wherein:
R^{X} is as defined in technical solution 29;
R^{4a} is selected from the group consisting of: C₂₋₆ alkenyl, C₂₋₆ haloalkenyl, C₂₋₆ alkynyl, and C₂₋₆ haloalkynyl, preferably, C₂₋₆ alkenyl, C₂₋₆ haloalkenyl, C₂₋₆ alkynyl, and C₂₋₆ haloalkynyl that containing one to three double bond or triple bond, preferably C₂₋₆ alkenyl, and C₂₋₆ haloalkenyl that containing cumulated double bonds, preferably C₂₋₆ alkenyl, and C₂₋₆ haloalkenyl that containing conjugate double bonds, preferably C₂₋₆ alkenyl, and C₂₋₆ haloalkenyl that containing independent double bonds, preferably, ethenyl, propenyl, ethynyl, and propynyl, preferably, ethenyl, and ethynyl;
R^{4b} is selected from the group consisting of: —H, —OH, —OR⁹, —OC(═O)R⁹, —NR^{e}R^{f}, and -halo, preferably, —F, —OH, —OR⁹, and —NR^{e}R^{f}, preferably, —F, —OH, —OMe, and —NH₂, preferably, —F, —OH, and —OMe;
R^{5b} is independently selected from the group consisting of:
   H, —OH, —SH, -halo, cyano, or azido;
   C₁₋₆ alkyl, C₁₋₆ haloalkyl, C₂₋₆ alkenyl, C₂₋₆ haloalkenyl, C₂₋₆ alkynyl, or C₂₋₆ haloalkynyl, each of which is optionally substituted with from 1-6 R^{a};
   —OR⁹, —NR^{e}R^{f};
preferably, R^{5b} is —OH;
L² is selected from the group consisting of: —O—, —S—, —NH—, —N(C₁₋₃ alkyl)-, —CH₂—, —CF₂—, —CHF—, —CH(C₁₋₃ alkyl)-, and —C(C₁₋₃ alkyl)OH—, preferably, —O—;
Y⁰ is selected from the group consisting of: —OH and —SH, preferably, —SH;
R² is selected from the group consisting of: -halo, —OH, —OR⁹, and —OC(═O)R⁹, preferably, -halo, —OH, or —OC(═O)R′, preferably, —OH, or —OC(═O)R⁹, preferably, —OH, or —OC(═O) C₁₋₆ alkyl, preferably, —OH;
R³ is selected from the group consisting of: —OH, —OR¹⁰, and —OC(═O)R¹⁰, preferably, —OH, or —OC(═O) C₁₋₂₀ alkyl, preferably, —OH;
R¹, R⁶, and R⁷ are each independently selected from the group consisting of: —OH, —OR⁹, and —OC(═O)R⁹, preferably, —OH, or —OC(═O) C₁₋₆ alkyl, preferably, —OH;
each occurrence of R⁹ is independently selected from the group consisting of: C₁₋₆ alkyl, and C₁₋₆ haloalkyl;
each occurrence of R¹⁰ is independently selected from the group consisting of: C₁₋₂₀ alkyl, C₁₋₂₀ haloalkyl, C₂₋₂₀ alkenyl, C₂₋₂₀ haloalkenyl, C₂₋₂₀ alkynyl, C₂₋₂₀ haloalkynyl, C₃₋₇ cycloalkyl, 3- to 7-membered heterocyclyl, C₆₋₁₀ aryl, and 5- to 10-membered heteroaryl;
each occurrence of R^{a} is independently selected from the group consisting of: —H; —OH; -halo; —NR^{e}R^{f}; C₁₋₄ alkoxy; C₁₋₄ haloalkoxy; —C(═O)O(C₁₋₄ alkyl); —C(═O) (C₁₋₄ alkyl); —C(═O)OH; —C(═O)NR′R″; —S(═O)₁₋₂NR′R″; —S(═O)₁₋₂ (C₁₋₄ alkyl); and cyano;
each occurrence of R^{e} and R^{f} is independently selected from the group consisting of: —H; C₁₋₆ alkyl or C₁₋₆ haloalkyl, which is optionally substituted with 1-3 substituents each independently selected from the group consisting of NR′R″, —OH, halo, C₁₋₄ alkoxy, and C₁₋₄ haloalkoxy; —C(═O)R′; —C(═O)OR′; —C(═O)NR′R″; C(═NR″)NR′R″; —C(═O) C(═O)R′; —S(═O)₁₋₂NR′R″; —S(═O)₁₋₂R′; —OH; and C₁₋₄ alkoxy; and
each occurrence of R′ and R″ is independently selected from the group consisting of: —H; C₁₋₄ alkyl or C₁₋₄ haloalkyl, which is optionally substituted with 1-3 substituents each independently selected from the group consisting of: halo, cyano, C₁₋₄ alkoxy, C₁₋₄ haloalkoxy, and —OH.

31. The compound of technical solution 28, wherein:
R″ is as defined in technical solution 29;
R^{4a} is selected from the group consisting of: C₂₋₆ alkenyl, C₂₋₆ haloalkenyl, C₂₋₆ alkynyl, and C₂₋₆ haloalkynyl, preferably, C₂₋₆ alkenyl, C₂₋₆ haloalkenyl, C₂₋₆ alkynyl, and C₂₋₆ haloalkynyl that containing one to three double bond or triple bond, preferably C₂₋₆ alkenyl, and C₂₋₆ haloalkenyl that containing cumulated double bonds, preferably C₂₋₆ alkenyl, and C₂₋₆ haloalkenyl that containing conjugate double bonds, preferably C₂₋₆ alkenyl, and C₂₋₆ haloalkenyl that containing independent double bonds, preferably, ethenyl, propenyl, ethynyl, and propynyl, preferably, ethenyl, and ethynyl;
R^{4b} is selected from the group consisting of: —OH, —OR⁹, —OC(═O)R⁹, —NR^{e}R^{f}, and -halo;
R^{5b} is selected from the group consisting of: —OH, —OR⁹, —NR^{e}R^{f}, and -halo;
L² is —O—;
Y⁰ is selected from the group consisting of: —OH and —SH;
R³ is selected from the group consisting of: H, D, -halo, —OH, —SH, cyano, —OR¹⁰, —OC(═O)R¹⁰, —NR^{e}R^{f}, —NR^{e}C(═O)R¹⁰, —OP(═O)(OR′)(OR″), —OS(═O)₁₋₂R¹⁰, C₁₋₆ alkyl, and C₁-6 haloalkyl;
R¹, R², R⁶, and R⁷ are each independently selected from the group consisting of: H, D, -halo, —OH, —SH, cyano, —OR⁹, —OC(═O)R⁹, —NR^{e}R^{f}, —NR^{e}C(═O)R⁹, —OP(═O)(OR′)(OR″), —OS(═O)₁₋₂R⁹, C₁₋₆ alkyl, and C₁₋₆ haloalkyl;
each occurrence of R⁹ is independently selected from the group consisting of: C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl, 3- to 7-membered heterocyclyl, C₆₋₁₀ aryl, and 5- to 10-membered heteroaryl;
each occurrence of R¹⁰ is independently selected from the group consisting of: C₁₋₂₀ alkyl, C₁₋₂₀ haloalkyl, C₂₋₂₀ alkenyl, C₂₋₂₀ haloalkenyl, C₂₋₂₀ alkynyl, C₂₋₂₀ haloalkynyl, C₃₋₇ cycloalkyl, 3- to 7-membered heterocyclyl, C₆₋₁₀ aryl, and 5- to 10-membered heteroaryl;
each occurrence of R^{e} and R^{f} is independently selected from the group consisting of: —H; C₁₋₆ alkyl or C₁₋₆ haloalkyl, which is optionally substituted with 1-3 substituents each independently selected from the group consisting of NR′R″, —OH, halo, C₁₋₄ alkoxy, and C₁₋₄ haloalkoxy; and —C(═O)R^{b}; and
each occurrence of R′ and R″ is independently selected from the group consisting of: —H; C₁₋₄ alkyl or C₁₋₄ haloalkyl, which is optionally substituted with 1-3 substituents each independently selected from the group consisting of: halo, cyano, C₁₋₄ alkoxy, C₁₋₄ haloalkoxy, and —OH.

32. The compound of technical solution 28, wherein:
R″ is as defined in technical solution 29;
R^{4a} is selected from the group consisting of: C₂₋₆ alkenyl, C₂₋₆ haloalkenyl, C₂₋₆ alkynyl, and C₂₋₆ haloalkynyl, preferably, C₂₋₆ alkenyl, C₂₋₆ haloalkenyl, C₂₋₆ alkynyl, and C₂₋₆ haloalkynyl that containing one to three double bond or triple bond, preferably C₂₋₆ alkenyl, and C₂₋₆ haloalkenyl that containing cumulated double bonds, preferably C₂₋₆ alkenyl, and C₂₋₆ haloalkenyl that containing conjugate double bonds, preferably C₂₋₆ alkenyl, and C₂₋₆ haloalkenyl that containing independent double bonds, preferably, ethenyl, propenyl, ethynyl, and propynyl, preferably, ethenyl, and ethynyl;
R^{4b} is selected from the group consisting of: —OH, —OR⁹, —NR^{e}R^{f}, and -halo, preferably —OH, and -halo;
R^{5b} is selected from the group consisting of: —OH, —OR⁹, and —NR^{e}R^{f};
L² is —O—;
Y⁰ is selected from the group consisting of: —OH and —SH;
R² is selected from the group consisting of: -halo, —OH, —OR⁹, and —OC(═O)R⁹;
R³ is selected from the group consisting of: —OH, —OR¹⁰, and —OC(═O)R¹⁰;
R¹, R⁶, and R⁷ are each independently selected from the group consisting of: —OH, —OR⁹, and —OC(═O)R⁹;
each occurrence of R⁹ is independently selected from the group consisting of: C₁₋₆ alkyl, and C₁₋₆ haloalkyl;
each occurrence of R¹⁰ is independently selected from the group consisting of: C₁₋₂₀ alkyl, C₁₋₂₀ haloalkyl, C₂₋₂₀ alkenyl, C₂₋₂₀ haloalkenyl, C₂₋₂₀ alkynyl, C₂₋₂₀ haloalkynyl, C₃₋₇ cycloalkyl, 3- to 7-membered heterocyclyl, C₆₋₁₀ aryl, and 5- to 10-membered heteroaryl;
each occurrence of R^{e} and R^{f} is independently selected from the group consisting of: —H; C₁₋₆ alkyl or C₁₋₆ haloalkyl, which is optionally substituted with 1-3 substituents each independently selected from the group consisting of NR′R″, —OH, halo, C₁₋₄ alkoxy, and C₁₋₄ haloalkoxy; and —C(═O)R^{b}; and
each occurrence of R′ and R″ is independently selected from the group consisting of: —H; C₁₋₄ alkyl or C₁₋₄ haloalkyl, which is optionally substituted with 1-3 substituents each independently selected from the group consisting of: halo, cyano, C₁₋₄ alkoxy, C₁₋₄ haloalkoxy, and —OH.

33. The compound of technical solution 28, wherein:
R^{X} is as defined in technical solution 29;
R^{4a} is C₂₋₆ alkenyl, C₂₋₆ haloalkenyl, C₂₋₆ alkynyl, or C₂₋₆ haloalkynyl, preferably, C₂₋₆ alkenyl, C₂₋₆ haloalkenyl, C₂₋₆ alkynyl, or C₂₋₆ haloalkynyl that containing one to three double bond or triple bond, preferably C₂₋₆ alkenyl, or C₂₋₆ haloalkenyl that containing cumulated double bonds, preferably C₂₋₆ alkenyl, or C₂₋₆ haloalkenyl that containing conjugate double bonds, preferably C₂₋₆ alkenyl, or C₂₋₆ haloalkenyl that containing independent double bonds, preferably, ethenyl, propenyl, ethynyl, or propynyl, preferably, ethenyl, or ethynyl;
R^{4b} is selected from the group consisting of: —OH, —OMe, —NH₂, and —F, preferably, —F;
R^{5b} is selected from the group consisting of: —OH, —NH₂, —NHMe, —NMe₂, and —NHAc, preferably, —OH;
L² is —O—;
Y⁰ is selected from the group consisting of: —OH and —SH, preferably-SH;
R² is selected from the group consisting of: —F, —OH, and —OAc, preferably —OH, or —OAc, preferably, —OH;
R³ is selected from the group consisting of: —OH, and —OC(═O) C₁₋₂₀ alkyl, preferably —OH, and —OAc, preferably, —OH;
R¹, R⁶, and R⁷ are each independently selected from the group consisting of: —OH, and —OAc, preferably, —OH.

34. The compound of technical solution 28, wherein:
R^{X} is selected from the group consisting of:

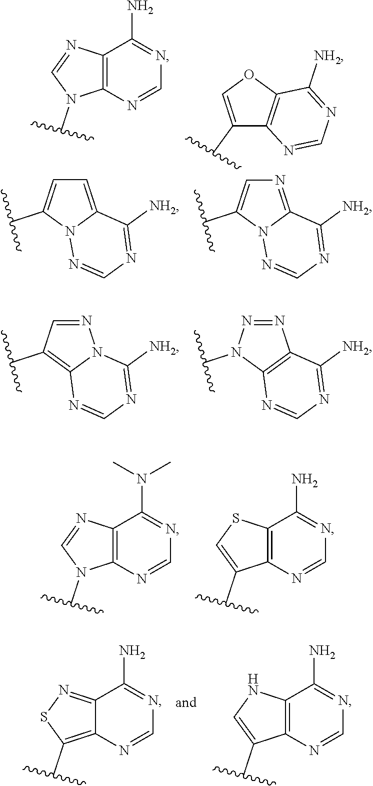

preferably,

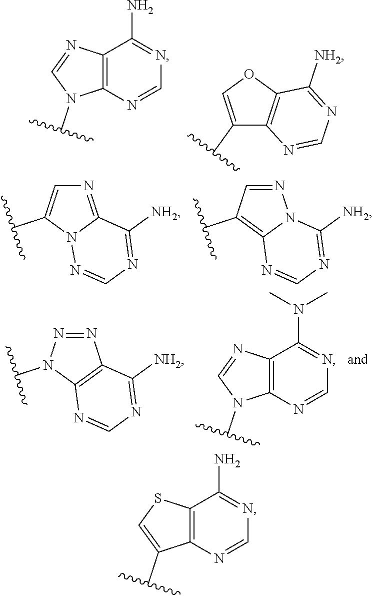

preferably,

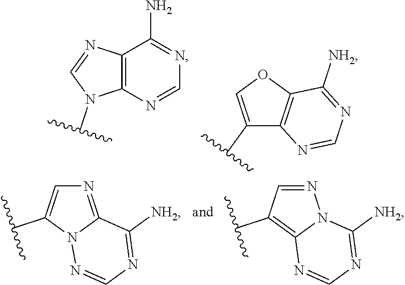

preferably,

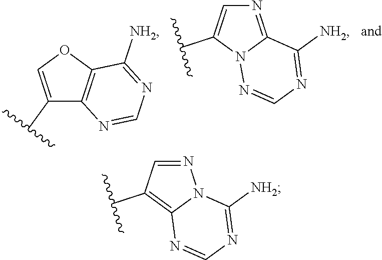

R^{4a} is C₂₋₆ alkenyl, or C₂₋₆ haloalkenyl, preferably, ethenyl, propenyl, ethynyl, or propynyl, preferably, ethenyl, or ethynyl, preferably, ethenyl;
R^{4b} is selected from the group consisting of: H, —OH, —OMe, —NH₂, and —F, preferably, —OH, and —F, preferably, —OH;
R^{5b} is selected from the group consisting of: —OH, —NH₂, —NHMe, —NMe₂, and —NHAc, preferably, —OH;
L² is —O—;
Y⁰ is selected from the group consisting of: —OH and —SH, preferably-SH;
R² is selected from the group consisting of: —F, —OH, and —OC(═O) C₁₋₆ alkyl, preferably —OH, or —OAc, preferably, —OAc;
R³ is selected from the group consisting of: —OH, and —OC(═O) C₁₋₂₀ alkyl, preferably —OH, and —OAc, preferably, —OAc;
R¹, R⁶, and R⁷ are each independently selected from the group consisting of: —OH, and —OAc, preferably, —OAc.

35. A compound of Formula (I-k), (I-k-1), (I-k-2), (I-k-3), (I-k-4) or (I-k-5):

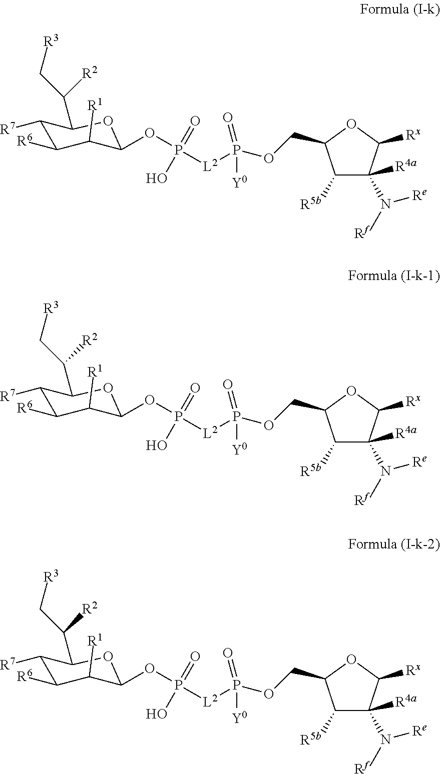

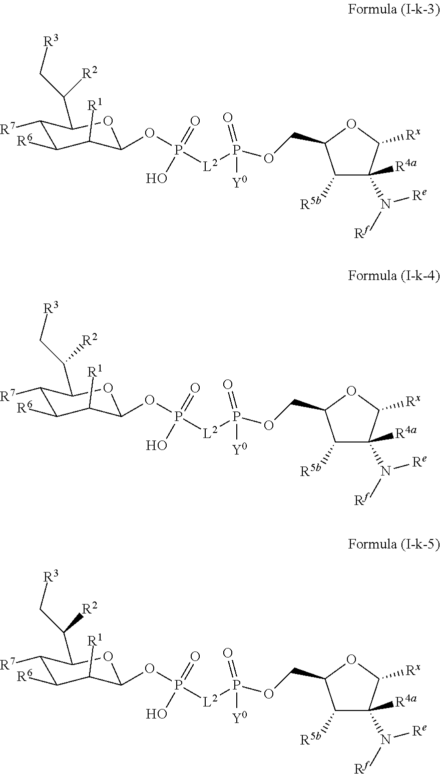

or a pharmaceutically acceptable salt, a stereoisomer, a stable isotope, a prodrug, or a tautomer thereof, wherein:
R^{X} is:
(A) a moiety having formula (X-la), (X-Ib), or (X-Ic):

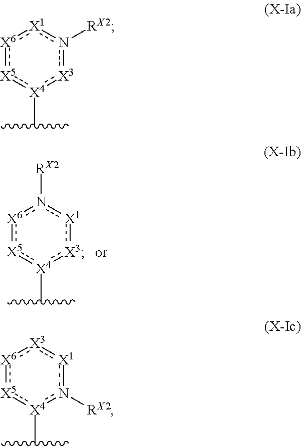

wherein:
X¹ is selected from the group consisting of: C(═O), C—OH, C═S, C—SH, C—NH₂, and C(═NH);
X³, X⁵, and X⁶ are each independently selected from the group consisting of: N, NH, N(R^{Xn}), CH, CR^{Xc}, C(═O), C(═S), C(═NH), and C(═NR^{Xn});
X⁴ is N or C;
R^{X2} is —H, R^{Xn}, or is absent when a double bond is present between NR^{X2} and an adjacent ring atom; and
each occurrence of [symbol] is independently a single bond or a double bond;
   provided that formulas (X-Ia), (X-Ib), and (X-Ic) each include from 1-2 endocyclic double bonds;
   provided that when X⁴ is C, then a double bond is present between X⁴ and an adjacent ring atom; and
   provided that when formulas (X-Ia), (X-Ib), and (X-Ic) each include only 1 endocyclic double bond, then X⁴ is N and/or one or more of X³, X⁵, and X⁶ are each independently selected from the group consisting of: N, NH, N(R^{Xn}), C(═O), C(═S), C(═NH), and C(═NR^{Xn});
   (B) pyridinyl, pyrmidinyl, pyrazinyl, pyridazinyl, or triazinyl each of which is optionally substituted with from 1-3 R^{Xc}, provided that any R^{Xc} group that is ortho or para to a ring nitrogen of (B) is other than —OH, —SH, or NH₂;
   (C) a moiety having formula (X-II):

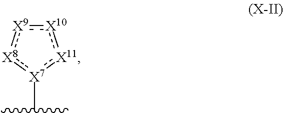

wherein:
X⁷ is C or N;
X⁸, X⁹, X¹⁰, and X¹¹ are each independently selected from the group consisting of: CH, C(R^{Xc}), N, N(H), N(R^{Xn}), O, S, C(═O), C(═S), C(═NH), and C(—NR^{Xn}); and
each [symbol] is independently a single bond or a double bond,
provided that from 1-4 of X⁷—X¹¹ is independently selected from group consisting of C, CH, C(R^{Xc}), C(═O), C(═S), C(═NH), and C(═NR^{Xn}), and (X-II) is aromatic;
   (D) C₆₋₁₀ aryl optionally substituted with from 1-4 R^{Xc}; or
   (E) bicyclic heteroaryl having 8-12 ring atoms, wherein from 1-5 ring atoms are heteroatoms each independently selected from the group consisting of: N, N(H), N(R^{Xn}), O, and S(—O)₀₋₂, and wherein one or more ring carbon atoms of the heteroaryl is optionally substituted with from 1-4 substituents each independently selected from the group consisting of oxo and R^{Xc};
each occurrence of R^{Xc} is independently selected from the group consisting of: R^{c}, R^{b}, and -(L^{b})_{b}-R^{b};
each occurrence of R^{Xn} is independently selected from the group consisting of: R^{d}, R^{b}, and -(L^{b})_{b}-R^{b};
R^{4a} and R^{5b} are independently selected from the group consisting of:
   H, —OH, —SH, -halo, cyano, or azido;
   C₁₋₆ alkyl, C₁₋₆ haloalkyl, C₂₋₆ alkenyl, C₂₋₆ haloalkenyl, C₂₋₆ alkynyl, or C₂₋₆ haloalkynyl, each of which is optionally substituted with from 1-6 R^{a};
   C₁₋₄ alkoxy or C₁₋₄ thioalkoxy, each of which is optionally substituted with from 1-6 R^{a};
   —OR⁹, —NR^{e}R^{f};
   —R^{b} or -(L^{b})_{b}-R^{b};
   —OP(═O)(OR′)(OR″); and
   —OC(═O) (C₁₋₆ alkyl) optionally substituted with from 1-6 R^{a};
L² is selected from the group consisting of: —O—, —S—, —NR^{L1}., and —C(R^{L2}) (R^{L2})—;
Y⁰ is selected from the group consisting of: —OH and —SH;
R³ is selected from the group consisting of: H, D, -halo, —OH, —SH, cyano, —OR¹⁰, —OC(═O)R¹⁰, —NR^{e}R^{f}, —NR^{e}C(═O)R¹⁰, —OP(=O)(OR′)(OR″), —OS(═O)₁₋₂R¹⁰, C₁₋₆ alkyl, and C₁₋₆ haloalkyl;
R¹, R², R⁶, and R⁷ are each independently selected from the group consisting of: H, D, -halo, —OH, —SH, cyano, —OR⁹, —OC(═O)R⁹, —NR^{e}R^{f}, —NR^{e}C(═O)R⁹, —OP(═O)(OR′)(OR″), —OS(═O)₁₋₂R⁹, C₁₋₆ alkyl, and C₁₋₆ haloalkyl;
each occurrence of R′ is independently selected from the group consisting of: C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl, 3- to 7-membered heterocyclyl, C₆₋₁₀ aryl, and 5- to 10-membered heteroaryl;
each occurrence of R¹⁰ is independently selected from the group consisting of: C₁₋₂₀ alkyl, C₁₋₂₀ haloalkyl, C₂₋₂₀ alkenyl, C₂₋₂₀ haloalkenyl, C₂₋₂₀ alkynyl, C₂₋₂₀ haloalkynyl, C₃₋₇ cycloalkyl, 3- to 7-membered heterocyclyl, C₆₋₁₀ aryl, and 5- to 10-membered heteroaryl;
each occurrence of R^{L1} is independently selected from the group consisting of: —H; C₁₋₆ alkyl, C₁₋₆ haloalkyl, C₂₋₆ alkenyl, C₂₋₆ haloalkenyl, C₂₋₆ alkynyl, or C₂₋₆ haloalkynyl, which is optionally substituted with 1-3 substituents each independently selected from the group consisting of NR′R″, —OH, C₁₋₄ alkoxy, and C₁₋₄ haloalkoxy; and —C(═O)R′;
each occurrence of R^{L2} is independently selected from the group consisting of: —H; -halo; —OH; —OR⁹; C₁₋₆ alkyl, C₁₋₆ haloalkyl, C₂₋₆ alkenyl, C₂₋₆ haloalkenyl, C₂₋₆ alkynyl, or C₂₋₆ haloalkynyl, which is optionally substituted with 1-3 substituents each independently selected from the group consisting of NR′R″, —OH, C₁₋₄ alkoxy, and C₁₋₄ haloalkoxy; and —C(═O)R′;
each occurrence of R^{a} is independently selected from the group consisting of: —H; —OH; -halo; —NR^{e}R^{f}; C₁₋₄ alkoxy; C₁₋₄ haloalkoxy; —C(═O)O(C₁₋₄ alkyl); —C(═O) (C₁₋₄ alkyl); —C(═O)OH; —C(═O)NR′R″; —S(═O)₁₋₂NR′R″; —S(═O)₁₋₂ (C₁₋₄ alkyl); and cyano;
each occurrence of R^{b} is independently selected from the group consisting of:
C₃₋₁₀ cycloalkyl or C₃₋₁₀ cycloalkenyl, each of which is optionally substituted with 1-4 R^{c};
heterocyclyl or heterocycloalkenyl of 3-10 ring atoms, wherein 1-3 ring atoms are heteroatoms, each independently selected from the group consisting of N, N(H), N(R^{d}), O, and S(═O)₀₋₂, and wherein the heterocyclyl or heterocycloalkenyl is optionally substituted with 1-4 R^{c};
heteroaryl of 5-10 ring atoms, wherein 1-3 ring atoms are heteroatoms, each independently selected from the group consisting of N, N(H), N(R^{d}), O, and S(═O)₀₋₂, and wherein the heteroaryl is optionally substituted with 1-4 R^{e}; and
C₆₋₁₀ aryl optionally substituted with 1-4 R^{c};
each occurrence of L^{b} is independently selected from the group consisting of: —O—, —NH—, —NR^{d}, —S(═O)₀₋₂, C(═O), and C₁₋₃ alkylene optionally substituted with 1-3 R^{a};
each occurrence of b is independently 1, 2, 3, or 4;
each occurrence of R^{e} is independently selected from the group consisting of: halo; cyano; C₁₋₁₀ alkyl which is optionally substituted with 1-6 independently selected R^{a}; C₂₋₆ alkenyl; C₂₋₆ alkynyl; C₁₋₄ alkoxy; C₁₋₄ haloalkoxy; —S(═O)₁₋₂ (C₁₋₄ alkyl); —NR^{e}R^{f}; —OH; —SH; —S(═O)₁₋₂NR′R″; —C₁₋₄ thioalkoxy; —NO₂; —OC(═O) (C₁₋₄ alkyl); —OC(═O) H; —C(═O) (C₁₋₄ alkyl); —C(═O) H; —C(═O)O(C₁₋₄ alkyl); —C(═O)OH; and —C(═O)NR′R″;
each occurrence of R^{d} is independently selected from the group consisting of: C₁₋₆ alkyl optionally substituted with 1-3 independently selected R^{a}; —C(═O) (C₁₋₄ alkyl); —C(═O)O(C₁₋₄ alkyl); —C(═O)NR′R″; —S(═O)₁₋₂NR′R″; —S(═O)₁₋₂ (C₁₋₄ alkyl); —OH; and C₁₋₄ alkoxy;
each occurrence of R^{e} and R^{f} is independently selected from the group consisting of: —H; C₁₋₆ alkyl or C₁₋₆ haloalkyl, which is optionally substituted with 1-3 substituents each independently selected from the group consisting of NR′R″, —OH, halo, C₁₋₄ alkoxy, and C₁₋₄ haloalkoxy; —C(═O)R′; —C(═O)OR′; —C(═O)NR′R″; C(═NR″)NR′R″; —C(═O) C(═O)R′; —S(═O)₁₋₂NR′R″; —S(═O)₁₋₂R′; —OH; and C₁₋₄ alkoxy; or
R^{e}, and R^{f} taken together with the N atom connecting them form a saturated or unsaturated 3- to 7-membered heterocyclyl; and
each occurrence of R′ and R″ is independently selected from the group consisting of: —H; C₁₋₄ alkyl or C₁₋₄ haloalkyl, which is optionally substituted with 1-3 substituents each independently selected from the group consisting of: halo, cyano, C₁₋₄ alkoxy, C₁₋₄ haloalkoxy, and —OH.

36. The compound of technical solution 35, wherein:

R^{X} is selected from the group consisting of:

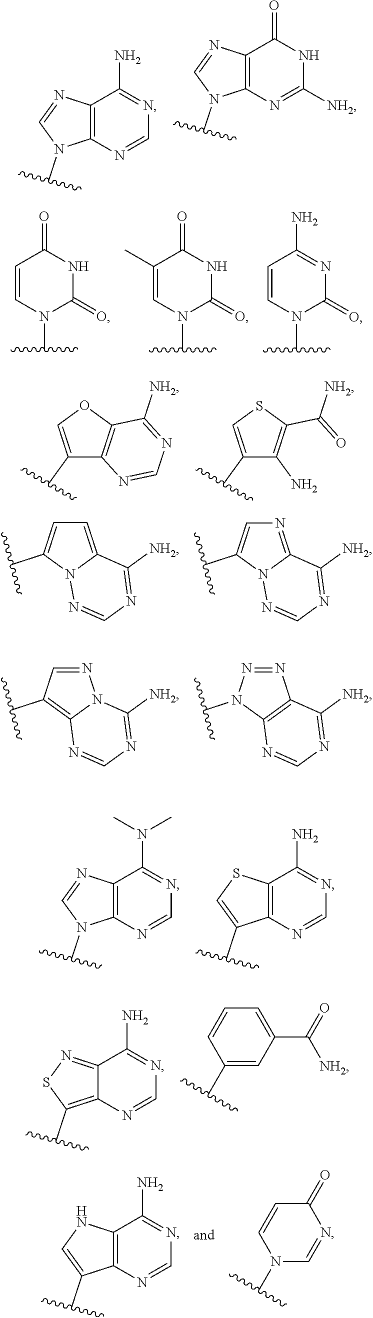

preferably,

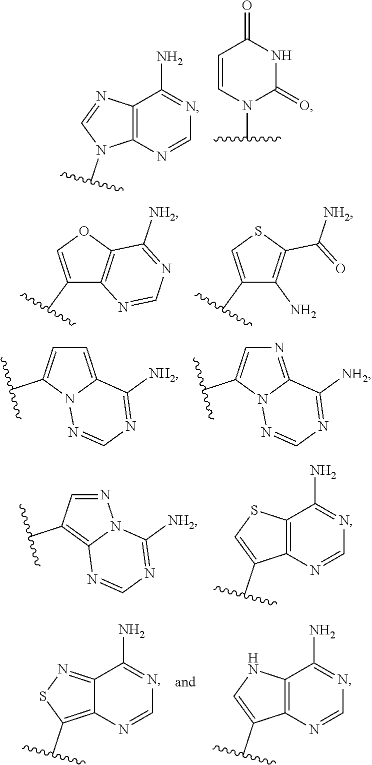

preferably,

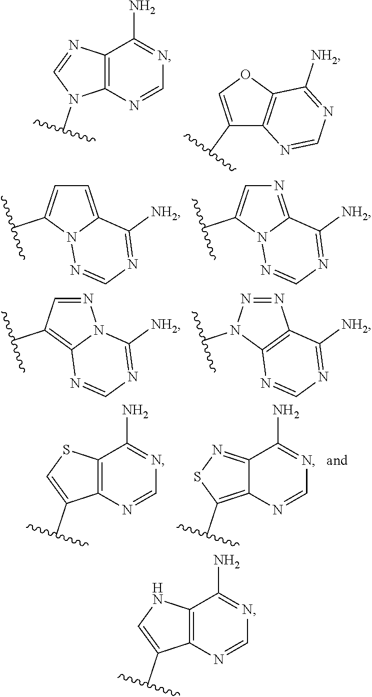

preferably,

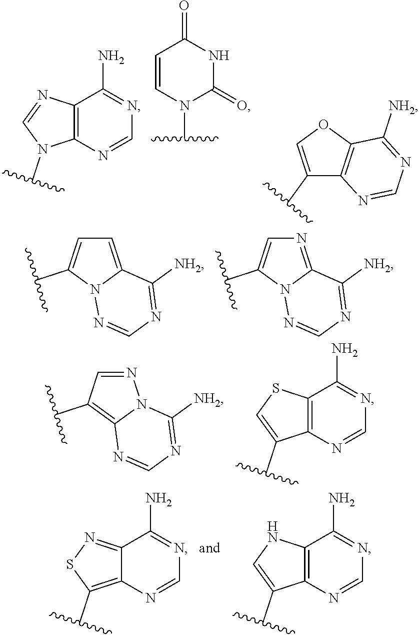

preferably,

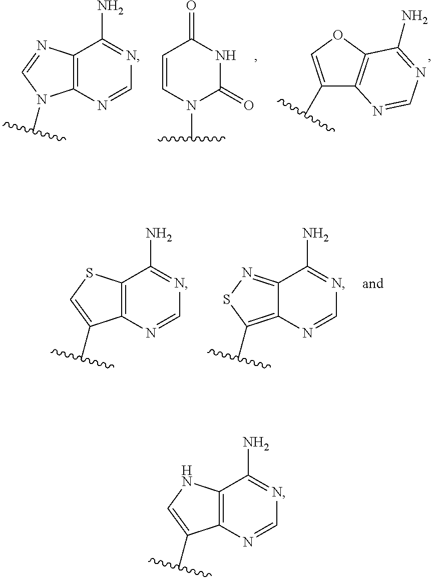

preferably,

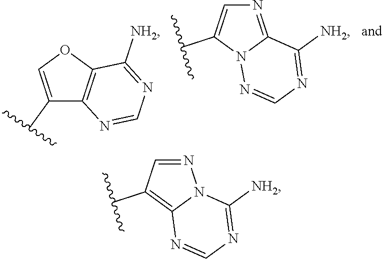

preferably,

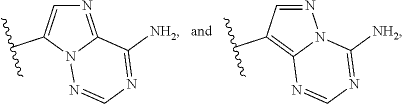

preferably,

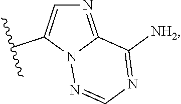 [SMILES: *c1cnc2c(N)ncnn12]

preferably,

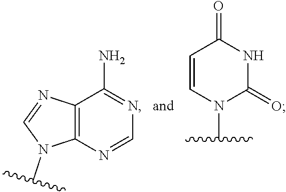

R^{4a} and R^{5b} are independently selected from the group consisting of:
   H, —OH, —SH, -halo, cyano, or azido;
   C₁₋₆ alkyl, C₁₋₆ haloalkyl, C₂₋₆ alkenyl, C₂₋₆ haloalkenyl, C₂₋₆ alkynyl, or C₂₋₆ haloalkynyl, each of which is optionally substituted with from 1-6 R^{a};
   C₁₋₄ alkoxy or C₁₋₄ thioalkoxy, each of which is optionally substituted with from 1-6 R^{a};
   —OR⁹, —NR^{e}R^{f};
   —OC(═O) (C₁₋₆ alkyl) optionally substituted with from 1-6 R^{a};
preferably, R^{4a} is —H, and R^{5b} is —OH;
L² is selected from the group consisting of: —O—, —S—, —NR^{L1}—, and —C(R^{L2}) (R^{L2})—;
Y⁰ is selected from the group consisting of: —OH and —SH;
R³ is selected from the group consisting of: H, D, -halo, —OH, —SH, cyano, —OR¹⁰, —OC(═O)R¹⁰, —NR^{e}R^{f}, —NR^{e}C(═O)R¹⁰, —OP(═O)(OR′)(OR″), —OS(═O)₁₋₂R¹⁰, C₁₋₆ alkyl, and C₁-6 haloalkyl;
R¹, R², R⁶, and R⁷ are each independently selected from the group consisting of: H, D, -halo, —OH, —SH, cyano, —OR⁹, —OC(═O)R⁹, —NR^{e}R^{f}, —NR^{e}C(═O)R′, —OP(═O)(OR′)(OR″), —OS(═O)₁₋₂R⁹, C₁₋₆ alkyl, and C₁₋₆ haloalkyl;
each occurrence of R⁹ is independently selected from the group consisting of: C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl, 3- to 7-membered heterocyclyl, C₆₋₁₀ aryl, or 5- to 10-membered heteroaryl;
each occurrence of R¹⁰ is independently selected from the group consisting of: C₁₋₂₀ alkyl, C₁₋₂₀ haloalkyl, C₂₋₂₀ alkenyl, C₂₋₂₀ haloalkenyl, C₂₋₂₀ alkynyl, C₂₋₂₀ haloalkynyl, C₃₋₇ cycloalkyl, 3- to 7-membered heterocyclyl, C₆₋₁₀ aryl, or 5- to 10-membered heteroaryl;
each occurrence of R^{L1} is independently selected from the group consisting of: —H; C₁₋₆ alkyl, C₁₋₆ haloalkyl, C₂₋₆ alkenyl, C₂₋₆ haloalkenyl, C₂₋₆ alkynyl, or C₂₋₆ haloalkynyl, which is optionally substituted with 1-3 substituents each independently selected from the group consisting of NR′R″, —OH, C₁₋₄ alkoxy, and C₁₋₄ haloalkoxy; and —C(═O)R′;
each occurrence of R^{L2} is independently selected from the group consisting of: —H; -halo; —OH; —OR⁹; C₁₋₆ alkyl, C₁₋₆ haloalkyl, C₂₋₆ alkenyl, C₂₋₆ haloalkenyl, C₂₋₆ alkynyl, or C₂₋₆ haloalkynyl, which is optionally substituted with 1-3 substituents each independently selected from the group consisting of NR′R″, —OH, C₁₋₄ alkoxy, and C₁₋₄ haloalkoxy; and —C(═O)R′;
each occurrence of R^{a} is independently selected from the group consisting of: —H; —OH; -halo; —NR^{e}R^{f}; C₁₋₄ alkoxy; C₁₋₄ haloalkoxy; —C(═O)O(C₁₋₄ alkyl); —C(═O) (C₁₋₄ alkyl); —C(═O)OH; —C(═O)NR′R″; —S(═O)₁₋₂NR′R″; —S(═O)₁₋₂ (C₁₋₄ alkyl); and cyano;
each occurrence of R^{e} and R^{f} is independently selected from the group consisting of: —H; C₁₋₆ alkyl or C₁₋₆ haloalkyl, which is optionally substituted with 1-3 substituents each independently selected from the group consisting of NR′R″, —OH, halo, C₁₋₄ alkoxy, and C₁₋₄ haloalkoxy; —C(═O)R′; —C(═O)OR′; —C(═O)NR′R″; C(═NR″)NR′R″; —C(═O) C(═O)R′; —S(═O)₁₋₂NR′R″; —S(═O)₁₋₂R′; —OH; and C₁₋₄ alkoxy; or
R^{e}, and R^{f} taken together with the N atom connecting them form a saturated or unsaturated 3- to 7-membered heterocyclyl; and
each occurrence of R′ and R″ is independently selected from the group consisting of: —H; C₁₋₄ alkyl or C₁₋₄ haloalkyl, which is optionally substituted with 1-3 substituents each independently selected from the group consisting of: halo, cyano, C₁₋₄ alkoxy, C₁₋₄ haloalkoxy, and —OH;
preferably, both R^{e} and R^{f} are C₁₋₆ alkyl, such as -Me.

37. The compound of technical solution 35, wherein:
R^{X} is as defined in technical solution 36;
R^{a} is selected from the group consisting of: —H, -halo, C₁₋₆ alkyl, C₁₋₆ haloalkyl, C₂₋₆ alkenyl, C₂₋₆ haloalkenyl, C₂₋₆ alkynyl, and C₂₋₆ haloalkynyl;
R^{5b} is independently selected from the group consisting of:
   H, —OH, —SH, -halo, cyano, or azido;
   C₁₋₆ alkyl, C₁₋₆ haloalkyl, C₂₋₆ alkenyl, C₂₋₆ haloalkenyl, C₂₋₆ alkynyl, or C₂₋₆ haloalkynyl, each of which is optionally substituted with from 1-6 R^{a};
   —OR⁹, —NR^{e}R^{f};
preferably, R^{4a} is —H, and R^{5b} is —OH;
L² is selected from the group consisting of: —O—, —S—, —NH—, —N(C₁₋₃ alkyl)-, —CH₂—, —CF₂—, —CHF—, —CH(C₁₋₃ alkyl)-, and —C(C₁₋₃ alkyl)OH—;
Y⁰ is selected from the group consisting of: —OH and —SH;
R² is selected from the group consisting of: -halo, —OH, —OR⁹, and —OC(═O)R⁹;
R³ is selected from the group consisting of: —OH, —OR¹⁰, and —OC(═O)R¹⁰;
R¹, R⁶, and R⁷ are each independently selected from the group consisting of: —OH, —OR⁹, and —OC(═O)R⁹;
each occurrence of R⁹ is independently selected from the group consisting of: C₁₋₆ alkyl, and C₁₋₆ haloalkyl;
each occurrence of R¹⁰ is independently selected from the group consisting of: C₁₋₂₀ alkyl, C₁₋₂₀ haloalkyl, C₂₋₂₀ alkenyl, C₂₋₂₀ haloalkenyl, C₂₋₂₀ alkynyl, C₂₋₂₀ haloalkynyl, C₃₋₇ cycloalkyl, 3- to 7-membered heterocyclyl, C₆₋₁₀ aryl, and 5- to 10-membered heteroaryl;
each occurrence of R^{a} is independently selected from the group consisting of: —H; —OH; -halo; —NR^{e}R^{f}; C₁₋₄ alkoxy; C₁₋₄ haloalkoxy; —C(═O)O(C₁₋₄ alkyl); —C(═O) (C₁₋₄ alkyl); —C(═O)OH; —C(═O)NR′R″; —S(═O)₁₋₂NR′R″; —S(—O)₁₋₂ (C₁₋₄ alkyl); and cyano;
each occurrence of R^{e} and R^{f} is independently selected from the group consisting of: —H; C₁₋₆ alkyl or C₁₋₆ haloalkyl, which is optionally substituted with 1-3 substituents each independently selected from the group consisting of NR′R″, —OH, halo, C₁₋₄ alkoxy, and C₁₋₄ haloalkoxy; —C(═O)R′; —C(═O)OR′; —C(═O)NR′R″; C(═NR″)NR′R″; —C(═O) C(═O)R′; —S(═O)₁₋₂NR′R″; —S(═O)₁₋₂R′; —OH; and C₁₋₄ alkoxy; and
each occurrence of R′ and R″ is independently selected from the group consisting of: —H; C₁₋₄ alkyl or C₁₋₄ haloalkyl, which is optionally substituted with 1-3 substituents each independently selected from the group consisting of: halo, cyano, C₁₋₄ alkoxy, C₁₋₄ haloalkoxy, and —OH;
preferably, both R^{e} and R^{f} are C₁₋₆ alkyl, such as -Me.

38. The compound of technical solution 35, wherein:
R^{X} is as defined in technical solution 36;
R^{4a} is selected from the group consisting of: —H, -halo, C₁₋₆ alkyl, C₁₋₆ haloalkyl, C₂₋₆ alkenyl, C₂₋₆ haloalkenyl, C₂₋₆ alkynyl, and C₂₋₆ haloalkynyl;
R^{5b} is selected from the group consisting of: —OH, —OR⁹, —NR^{e}R^{f}, and -halo;
L² is —O—;
Y⁰ is selected from the group consisting of: —OH and —SH;
R³ is selected from the group consisting of: H, D, -halo, —OH, —SH, cyano, —OR¹⁰, —OC(═O)R¹⁰, —NR^{e}R^{f}, —NR^{e}C(═O)R¹⁰, —OP(═O)(OR′)(OR″), —OS(—O)₁₋₂R¹⁰, C₁₋₆ alkyl, and C₁₋₆ haloalkyl;
R¹, R², R⁶, and R⁷ are each independently selected from the group consisting of: H, D, -halo, —OH, —SH, cyano, —OR⁹, —OC(═O)R⁹, —NR^{e}R^{f}, —NR^{e}C(═O)R⁹, —OP(—O)(OR′)(OR″), —OS(═O)₁₋₂R⁹, C₁₋₆ alkyl, and C₁₋₆ haloalkyl;
each occurrence of R⁹ is independently selected from the group consisting of: C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl, 3- to 7-membered heterocyclyl, C₆₋₁₀ aryl, and 5- to 10-membered heteroaryl;
each occurrence of R¹⁰ is independently selected from the group consisting of: C₁₋₂₀ alkyl, C₁₋₂₀ haloalkyl, C₂₋₂₀ alkenyl, C₂₋₂₀ haloalkenyl, C₂₋₂₀ alkynyl, C₂₋₂₀ haloalkynyl, C₃₋₇ cycloalkyl, 3- to 7-membered heterocyclyl, C₆₋₁₀ aryl, and 5- to 10-membered heteroaryl;
each occurrence of R^{e} and R^{f} is independently selected from the group consisting of: —H; C₁₋₆ alkyl or C₁₋₆ haloalkyl, which is optionally substituted with 1-3 substituents each independently selected from the group consisting of NR′R″, —OH, halo, C₁₋₄ alkoxy, and C₁₋₄ haloalkoxy; and —C(═O)R^{b}; and
each occurrence of R′ and R″ is independently selected from the group consisting of: —H; C₁₋₄ alkyl or C₁₋₄ haloalkyl, which is optionally substituted with 1-3 substituents each independently selected from the group consisting of: halo, cyano, C₁₋₄ alkoxy, C₁₋₄ haloalkoxy, and —OH;
preferably, both R^{e} and R^{f} are C₁₋₆ alkyl, such as -Me.

39. The compound of technical solution 35, wherein:
R^{X} is as defined in technical solution 36;
R^{4a} is selected from the group consisting of: —H, -halo, C₁₋₆ alkyl, C₁₋₆ haloalkyl, C₂₋₆ alkenyl, C₂₋₆ haloalkenyl, C₂₋₆ alkynyl, and C₂₋₆ haloalkynyl;
R^{5b} is selected from the group consisting of: —OH, —OR⁹, and —NR^{e}R^{f};
L² is —O—;
Y⁰ is selected from the group consisting of: —OH and —SH;
R² is selected from the group consisting of: -halo, —OH, —OR⁹, and —OC(═O)R⁹;
R³ is selected from the group consisting of: —OH, —OR¹⁰, and —OC(═O)R¹⁰;
R¹, R⁶, and R⁷ are each independently selected from the group consisting of: —OH, —OR⁹, and —OC(═O)R⁹;
each occurrence of R⁹ is independently selected from the group consisting of: C₁₋₆ alkyl, and C₁₋₆ haloalkyl;
each occurrence of R¹⁰ is independently selected from the group consisting of: C₁₋₂₀ alkyl, C₁₋₂₀ haloalkyl, C₂₋₂₀ alkenyl, C₂₋₂₀ haloalkenyl, C₂₋₂₀ alkynyl, C₂₋₂₀ haloalkynyl, C₃₋₇ cycloalkyl, 3- to 7-membered heterocyclyl, C₆₋₁₀ aryl, and 5- to 10-membered heteroaryl;
each occurrence of R^{e} and R^{f} is independently selected from the group consisting of: —H; C₁₋₆ alkyl or C₁₋₆ haloalkyl, which is optionally substituted with 1-3 substituents each independently selected from the group consisting of NR′R″, —OH, halo, C₁₋₄ alkoxy, and C₁₋₄ haloalkoxy; and —C(═O)R^{b}; and
each occurrence of R′ and R″ is independently selected from the group consisting of: —H; C₁₋₄ alkyl or C₁₋₄ haloalkyl, which is optionally substituted with 1-3 substituents each independently selected from the group consisting of: halo, cyano, C₁₋₄ alkoxy, C₁₋₄ haloalkoxy, and —OH;
preferably, both R^{e} and R^{f} are C₁₋₆ alkyl, such as -Me.

40. The compound of technical solution 35, wherein:
R^{X} is as defined in technical solution 36;
R^{4a} is —H, or Me, preferably-H;
R^{5b} is selected from the group consisting of: —OH, —NH₂, —NHMe, —NMe₂, and —NHAc, preferably —OH;
L² is —O—;
Y⁰ is selected from the group consisting of: —OH and —SH, preferably-SH;
R² is selected from the group consisting of: —F, —OH, and —OAc, preferably —OH;
R¹, R³, R⁶, and R⁷ are each independently selected from the group consisting of: —OH, and —OAc, preferably —OH;
each occurrence of R^{e} and R^{f} is-H; C₁₋₆ alkyl or —C(═O) C₁₋₄ alkyl, preferably-H or C₁₋₆ alkyl; preferably, both R^{e} and R^{f} are C₁₋₆ alkyl, such as -Me.

41. The compound of technical solution 35, wherein:

R′ is selected from the group consisting of:

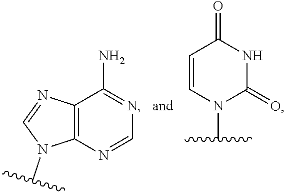

preferably

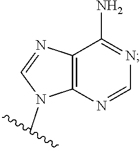 [SMILES: *n1cnc2c(N)ncnc21]

R^{4a} is —H, or Me, preferably-H;
R^{5b} is selected from the group consisting of: —OH, —NH₂, —NHMe, —NMe₂, and —NHAc, preferably —OH;
L² is —O—;
Y⁰ is selected from the group consisting of: —OH and —SH, preferably-SH;
R² is selected from the group consisting of: —F, —OH, and —OAc, preferably —OAc;
R³ is selected from the group consisting of: —OH, and —OC(═O) C₁₋₂₀ alkyl, preferably —OH, and —OAc, preferably, —OAc;
R¹, R⁶, and R⁷ are each independently selected from the group consisting of: —OH, and —OAc, preferably-OAc;
each occurrence of R^{e} and R^{f} is-H; C₁₋₆ alkyl or —C(═O) C₁₋₄ alkyl, preferably-H or C₁₋₆ alkyl; preferably, both R^{e} and R^{f} are C₁₋₆ alkyl, such as -Me.

42. A compound selected from the group consisting of the compounds delineated in Table 1, or a pharmaceutically acceptable salt, a stereoisomer, a stable isotope, a prodrug, or a tautomer thereof.

43. A pharmaceutical composition, comprising:
the compound, or the pharmaceutically acceptable salt, the stereoisomer, the stable isotope, the prodrug, or the tautomer thereof according to any one of technical solutions 1-42;
pharmaceutically acceptable excipient(s); and
optionally, one or more other therapeutic agents.

44. A kit, comprising:
a first container which contains the compound, or the pharmaceutically acceptable salt, the stereoisomer, the stable isotope, the prodrug, or the tautomer thereof according to any one of technical solutions 1-42; and
optionally, a second container which contains one or more other therapeutic agents; and
optionally, a third container which contains pharmaceutically acceptable excipient(s) for diluting or suspending the said compound and/or other therapeutic agent(s).

45. Use of a compound, or a pharmaceutically acceptable salt, a stereoisomer, a stable isotope, a prodrug, or a tautomer thereof according to any one of technical solutions 1-42, in the manufacture of a medicament for treating an immune and/or inflammatory related disease.

46. A compound, or a pharmaceutically acceptable salt, a stereoisomer, a stable isotope, a prodrug, or a tautomer thereof according to any one of technical solutions 1-42, for use in treating an immune and/or inflammatory related disease.

47. A method of treating an immune and/or inflammatory related disease in a subject in need thereof, comprising administering to the subject a therapeutically effective amount of a compound, or a pharmaceutically acceptable salt, a stereoisomer, a stable isotope, a prodrug, or a tautomer thereof according to any one of technical solutions 1-42.

48. The use of technical solution 45 or the compound for use of technical solution 46 or the method of technical solution 47, wherein the immune and/or inflammatory related disease is inflammatory bowel disease.

49. The use of technical solution 45 or the compound for use of technical solution 46 or the method of technical solution 47, wherein the immune and/or inflammatory related disease is ulcerative colitis.

50. The use of technical solution 45 or the compound for use of technical solution 46 or the method of technical solution 47, wherein the immune and/or inflammatory related disease is Crohn's disease.

51. Use of a compound, or a pharmaceutically acceptable salt, a stereoisomer, a stable isotope, a prodrug, or a tautomer thereof according to any one of technical solutions 1-42, in the manufacture of a medicament for treating a cancer.

52. A compound, or a pharmaceutically acceptable salt, a stereoisomer, a stable isotope, a prodrug, or a tautomer thereof according to any one of technical solutions 1-42, for use in treating a cancer.

53. A method of treating a cancer in a subject in need thereof, comprising administering to the subject a therapeutically effective amount of a compound, or a pharmaceutically acceptable salt, a stereoisomer, a stable isotope, a prodrug, or a tautomer thereof according to any one of technical solutions 1-42.

54. The use of technical solution 51 or the compound for use of technical solution 52 or the method of technical solution 53, wherein the cancer is selected from the group consisting of brain cancer, skin cancer, bladder cancer, ovarian cancer, breast cancer, gastric cancer, pancreatic cancer, hepatocellular cancer, prostate cancer, colorectal cancer, blood cancer, lung cancer, and bone cancer.

55. The use of technical solution 51 or the compound for use of technical solution 52 or the method of technical solution 53, wherein the cancer is selected from the group consisting of: small cell lung cancer, non-small cell lung cancer, colorectal cancer, melanoma, renal cell carcinoma, head and neck cancer, Hodgkin's lymphoma, and bladder cancer.

56. Use of a compound, or a pharmaceutically acceptable salt, a stereoisomer, a stable isotope, a prodrug, or a tautomer thereof according to any one of technical solutions 1-42, in the manufacture of a medicament for enhancing the efficacy of a vaccine.

57. A compound, or a pharmaceutically acceptable salt, a stereoisomer, a stable isotope, a prodrug, or a tautomer thereof according to any one of technical solutions 1-42, for use in enhancing the efficacy of a vaccine.

58. A method of enhancing the efficacy of a vaccine in a subject in need thereof, comprising administering to the subject a therapeutically effective amount of a compound, or a pharmaceutically acceptable salt, a stereoisomer, a stable isotope, a prodrug, or a tautomer thereof according to any one of technical solutions 1-42.

59. The use of technical solution 56 or the compound for use of technical solution 57 or the method of technical solution 58, wherein the vaccine is a cancer vaccine.

60. The use of technical solution 56 or the compound for use of technical solution 57 or the method of technical solution 58, wherein the vaccine is a bacterial vaccine.

61. The use of technical solution 56 or the compound for use of technical solution 57 or the method of technical solution 58, wherein the vaccine is a viral vaccine.

62. The use of technical solution 56 or the compound for use of technical solution 57 or the method of technical solution 58, wherein the vaccine is a parasite vaccine.

63. The use of technical solution 56 or the compound for use of technical solution 57 or the method of technical solution 58, wherein the compound is an adjuvant.

64. Use of a compound, or a pharmaceutically acceptable salt, a stereoisomer, a stable isotope, a prodrug, or a tautomer thereof according to any one of technical solutions 1-42, in the manufacture of a medicament for enhancing innate immunity.

65. A compound, or a pharmaceutically acceptable salt, a stereoisomer, a stable isotope, a prodrug, or a tautomer thereof according to any one of technical solutions 1-42, for use in enhancing innate immunity.

66. A method of enhancing innate immunity in a subject in need thereof, comprising administering to the subject a therapeutically effective amount of a compound, or a pharmaceutically acceptable salt, a stereoisomer, a stable isotope, a prodrug, or a tautomer thereof according to any one of technical solutions 1-42.

67. The use of technical solution 64 or the compound for use of technical solution 65 or the method of technical solution 66, wherein administering comprises intramuscular, intraperitoneal, intratumoral, or intravenous administration.

68. The use of technical solution 64 or the compound for use of technical solution 65 or the method of technical solution 66, wherein administering further comprises one or more immunotherapeutic agents.

69. The use of technical solution 64 or the compound for use of technical solution 65 or the method of technical solution 66, wherein the one or more immunotherapeutic agents comprises a small molecule, an antibody, or a cytokine.

### Pharmaceutical Compositions and Administration

### General

In some embodiments, a chemical entity (e.g., a compound that modulates (e.g., agonizes) ALPK1, or a pharmaceutically acceptable salt, and/or hydrate, and/or cocrystal, and/or stable isotope, and/or prodrug, and/or drug combination thereof) is administered as a pharmaceutical composition that includes the chemical entity and one or more pharmaceutically acceptable excipients, and optionally one or more additional therapeutic agents as described herein.

In some embodiments, the chemical entities can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22nd Edition (Pharmaceutical Press, London, U K. 2012).

### Routes of Administration and Composition Components

In some embodiments, the chemical entities described herein or a pharmaceutical composition thereof can be administered to subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In certain embodiments, a preferred route of administration is parenteral (e.g., intratumoral).

Compositions can be formulated for parenteral administration, e.g., formulated for injection via the intravenous, intramuscular, sub-cutaneous, or even intraperitoneal routes.

Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Intratumoral injections are discussed, e.g., in Lammers, et al., “Effect of Intratumoral Injection on the Biodistribution and the Therapeutic Potential of HPMA Copolymer-Based Drug Delivery Systems” Neoplasia. 2006, 10, 788-795.

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the chemical entities described herein with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In other embodiments, the compounds described herein or a pharmaceutical composition thereof are suitable for local delivery to the digestive or GI tract by way of oral administration (e.g., solid or liquid dosage forms.).

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain embodiments, solid oral dosage forms can further include one or more components that chemically and/or structurally predispose the composition for delivery of the chemical entity to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802, which is incorporated herein by reference in its entirety.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

In any of the foregoing embodiments, pharmaceutical compositions described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

### Dosages

The dosages may be varied depending on the requirement of the patient, the severity of the condition being treating and the particular compound being employed. Determination of the proper dosage for a particular situation can be determined by one skilled in the medical arts. The total daily dosage may be divided and administered in portions throughout the day or by means providing continuous delivery.

In some embodiments, the compounds described herein are administered at a dosage of from about 0.001 mg/Kg to about 500 mg/Kg (e.g., from about 0.001 mg/Kg to about 200 mg/Kg; from about 0.01 mg/Kg to about 200 mg/Kg; from about 0.01 mg/Kg to about 150 mg/Kg; from about 0.01 mg/Kg to about 100 mg/Kg; from about 0.01 mg/Kg to about 50 mg/Kg; from about 0.01 mg/Kg to about 10 mg/Kg; from about 0.01 mg/Kg to about 5 mg/Kg; from about 0.01 mg/Kg to about 1 mg/Kg; from about 0.01 mg/Kg to about 0.5 mg/Kg; from about 0.01 mg/Kg to about 0.1 mg/Kg; from about 0.1 mg/Kg to about 200 mg/Kg; from about 0.1 mg/Kg to about 150 mg/Kg; from about 0.1 mg/Kg to about 100 mg/Kg; from about 0.1 mg/Kg to about 50 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg).

### Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a therapeutic compound is administered to an individual for a period of time followed by a separate period of time. In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, months, 6 months, 7 months, 8 months, 9 months, 10 months, 11 months, 12 months, or more.

### Immunogenic Compositions

In another aspect, this disclosure provides immunogenic compositions (e.g., vaccines) that include (i) one or more agents (e.g., one or more antigens) that elicit an immunological response in a subject (e.g., a human or animal subject) and (ii) one or more adjuvants having Formulae described herein.

In another aspect, this disclosure provides immunogenic combinations as one or more kits or packs. In certain embodiments, the kit or pack includes two or more separately contained/packaged components, e.g. two components, which when mixed, provide the desired immunogenic compositions as described herein. In certain of these embodiments, the two-component system includes a first component and a second component, in which: (i) the first component is a vaccine and (ii) the second component includes one or more adjuvants having Formulae described herein. 15 In some embodiments, the immunological response observed is greater than the immunological response observed in the absence of the one or more adjuvants.

In some embodiments, immunological response stimulates the subject's (e.g., a human or animal subject's) immune system to produce immunity to a specific disease or condition

In some embodiments, the immunological response can be a cellular and/or antibody-mediated immune response to the immunogenic compositions described herein. For example, an “immunological response” includes but is not limited to one or more of the following effects: the production or activation of antibodies, B cells, helper T cells, suppressor T cells, and/or cytotoxic T cells and/or gamma-delta T cells, directed specifically to an antigen or antigens included in the immunogenic compositions described herein. Preferably, the subject will display either a protective immunological response or a therapeutically effective response.

A “protective immunological response” can be demonstrated by either a reduction or lack of clinical signs normally displayed by an infected host, a quicker recovery time and/or a lowered duration of infectivity or lowered pathogen titer in the tissues or body fluids or excretions of the infected host.

In some embodiments, the one or more agents that elicit an immunological response in a subject are one or more antigens.

In some embodiments, the “vaccine” is a pharmaceutical preparation used for the purpose of the prevention of infection and contains a deactivated or attenuated antigen. The vaccine can induce the immune response when administered to a human or animal subject and prevent the infection (including allergic reactions) with the antigen contained in the vaccine and the aggravation of the infection after the induction. A vaccine typically contains an agent resembling a disease-causing microorganism and typically made from weakened or killed forms of the microbe, its toxins, or one of its surface proteins. While not wishing to be bound by theory, it is believed that the agent stimulates the body's immune system to recognize the agent as a threat, destroy it, and to further recognize and destroy any of the microorganisms associated with that agent that it may encounter in the future. Vaccines can be prophylactic (to prevent or ameliorate the effects of a future infection by a natural or “wild” pathogen), or therapeutic (to fight a disease that has already occurred, such as cancer).

As used herein, the term “adjuvant” means a substance that is administered with an antigen and thereby increases the antigenicity of the antigen to facilitate the induction of immune response.

As used herein, the term “antigen” refers to a generic term for foreign substances, or a part thereof, that enter the living body from the outside and cause the immune response in the living body (e.g., a toxin or other foreign substance, which induces an immune response in the body, especially the production of antibodies.). The antigens include exogenous pathogens such as bacteria and viruses that cause various infections as well as allergens, which cause the allergic reaction among pollens, foods, and the like.

### Antigens

When administered to a subject, antigens generally specifically interact with an antigen recognition molecule of the immune system such as, e.g., an immunoglobulin (antibody) or a T cell antigen receptor (TCR) to elicit an immune response leading to the generation of a cell response (e.g., memory cells (e.g., memory B- and T-cells) or cytotoxic cells) and/or a humoral (antibody) response.

An “antigen” as used herein refers to, but is not limited to, components which elicit an immunological response in a host to an immunogenic composition or vaccine of interest comprising such antigen or an immunologically active component thereof. The antigen or immunologically active component can be a whole microorganism (in inactivated or modified live form), or any fragment or fraction thereof, which, if administered to a host, can elicit an immunological response in the host. The antigen can be or can comprise complete live organisms in either its original form or as attenuated organisms in a so called modified live vaccine (MLV). The antigen can further comprise appropriate elements of said organisms (subunit vaccines) whereby these elements are generated either by destroying the whole organism or the growth cultures of such organisms and subsequent purification steps yielding in the desired structure(s), or by synthetic processes induced by an appropriate manipulation of a suitable system like, but not restricted to bacteria, insects, mammalian or other species, and optionally by subsequent isolation and purification procedures, or by induction of said synthetic processes in the animal needing a vaccine by direct incorporation of genetic material using suitable pharmaceutical compositions (polynucleotide vaccination). The antigen can comprise whole organisms inactivated by appropriate methods in a so called killed vaccine (KV). If the organism is a bacterium, the killed vaccine is called a bacterin.

The compositions, combinations, and methods described herein may be used with any type of antigens such as, without limitation, whole pathogens (such as cells, viruses) or fragments or fractions thereof (such as proteins, polypeptides, peptides, nucleic acids, lipids, etc.). The pathogen may be any agent capable of infecting an animal, for example, a human, avian (e.g., chicken, turkey, duck, pigeon, etc.), canine, feline, bovine, porcine, or equine. The antigen may be e.g., a whole pathogen, a “surface antigen” expressed naturally e.g., on the surface of a pathogen or of an infected or diseased (e.g. tumor) cell.

More particularly, the antigen may be any pathogenic, or not, microorganisms, such as viruses, bacteria, any other parasites, or antigens. These may be live, attenuated, inactivated, or killed microorganisms, either whole microorganisms or microorganisms' subunits, inactivated chimeric or recombinant microorganisms, disrupted microorganisms, mutant microorganisms, defective microorganisms, or combinations thereof. The antigen may also be or include one or more epitopes or antigenic parts of the whole microorganism structure, e.g., virus, bacteria or parasite, such as preparations of antigenic proteins from pathogens, recombinant proteins, preferably viral antigen, such as viral capsid proteins, cell wall proteins, peptides, or parts of bacterial or parasite structure, such as polysaccharides, lipopolysaccharides and glycoproteins. The antigen may also be a DNA or recombinant DNA. Antigens may be provided in a purified or an unpurified form.

When the antigen is an attenuated microorganism, such as a virus, bacterium or other pathogens, the attenuated pathogen retains immunogenic properties and is essentially devoid of pathogenic properties. Attenuation can come from natural or artificial attenuation processes such as passages in living animals or various natural media including organs, cells, embryonated eggs, etc. Artificial attenuation can also be obtained by chemical treatment, drying, aging, adaptation to low temperatures or particular conditions of culture, genetic deletions, etc.

The antigen may also comprise killed inactivated microorganisms. Preparation of inactivated viruses for vaccination is generally achieved via chemical or physical means. Chemical inactivation can be effected by treating viruses for example with enzymes, formaldehyde, .beta.-propiolactone, Binary ethylene-imine or a derivative thereof. Inactivated virus so obtained may be neutralized or stabilized afterwards. Physical inactivation may be carried out by subjecting viruses to energy-rich radiation, such as UV-light, X-radiation or .gamma.-radiation.

Bacteria, including spores, can be inactivated e.g., by heat, pressure and/or the use of chemical agents often referred to as bacteriocides. For example, corrosive compositions, e.g., formaldehyde and sodium hypochlorite (bleach), have been used to inactivate bacteria. Alternatively, inactivation of bacteria may be obtained by ethylene oxide exposure, g-irradiation, steam sterilization, or by using near- and supercritical carbon dioxide treatment. The bacteria may also by inactivated or rendered avirulant by genetic modification of one or several genes involved in pathogenicity. Examples of such genetic modifications are disclosed for instance in WO2012/092226.

Such attenuated or inactivated microorganisms, e.g., viruses, bacteria or other avian parasites may also be purchased from commercial sources.

The antigen may be homologous or heterologous types.

Vaccines or compositions of the invention may comprise a combination of live antigens, synthetic antigens, fragments or fractions thereof. The compositions may also comprise antigens from various pathogens, to provide broad immune response.

Antigens may be (derived from) viruses responsible for common diseases as described by G. D. Butcher, J. P. Jacob, and F. B. Mather (PS47, Veterinary Medicine-Large Animal Clinical Sciences Department, Florida Cooperative Extension Service, Institute of Food and Agricultural Sciences, University of Florida; May 1999) such as Avian Pox, Newcastle Disease, Infectious Bronchitis, Quail Bronchitis, Lymphoid Leukosis, Marek's Disease, Infectious Bursal Disease, Infectious Laryngo tracheitis, Egg Drop Syndrome, Reovirosis, Infectious Tenosynovitis, Avian Encephalomyelitis, Swollen Head Syndrome, Turkey Rhinotracheitis or Avian Influenza, from bacteria responsible for mycoplasmosis, pasteurellosis, salmonellosis, bordetellosis, etc., and/or from other avian parasites responsible for coccidiosis, campylobacteriosis. Preferred vaccine used in the vaccine composition of the present invention comprises whole attenuated live virus strain.

Non-limiting examples of the viral antigens include an inactivated or attenuated preparation(s) of at least one virus selected from the group consisting of influenza virus, norovirus, rotavirus, human papillomavirus, varicella virus, measles virus, mumps virus, poliovirus, adenovirus, herpesvirus, human coronavirus, rubella virus, HIV, smallpox virus, Ebola virus, hepatitis virus, Japanese encephalitis virus, parvovirus, coronavirus, Zika and cowpox virus, or a part or a component thereof.

In some embodiments, the antigen is an antigen from at least one virus that causes hand, foot, and mouth disease in humans, such as EV71, CA6, and CA16. Advantageously, the antigens may include at least one adaptation mutation that allows for production in cultured non-human cell lines such as Vero cells. Moreover, as disclosed herein, the vaccines and immunogenic compositions of the present disclosure have been demonstrated to induce a protective immune response against viruses that cause hand, foot, and mouth disease in humans.

In some embodiments, the antigen is a PPV viral protein 2 (VP2) antigen.

In some embodiments, the antigen is a coronavirus antigen. MERS-COV antigens include viral antigens encoded by the structural protein genes Spike(S), Envelope (E), Membrane (M) and nucleopcapside (N). MERS-COV also expresses a polymerase. Spike(S) protein is assembled into trimers which form peplomers on the surface of the viral particle that give the Coronaviridae family its name. Typically, an immunogen or vaccine containing a CD40-targeted polypeptide (a polypeptide that is directed or targeted to CD40 on antigen presenting cells) of the invention will only contain viral S protein, or only S1 protein epitopes, for example, it will omit epitopes from other MERS-COV antigens and a S1-specific immunogen will omit S2 epitopes. However, in some embodiments, such a vaccine may substitute or include one or more other antigens or epitopes of non-S1 MERS-COV antigens either as part of a CD40-targeted polypeptide or as a separate ingredient of an immunogenic composition or vaccine.

In addition to MERS-COV, other types of human coronaviruses are known. These are 229E (alpha coronavirus), NL63 (alpha coronavirus), OC43 (beta coronavirus), HKU1 (beta coronavirus) and SARS-COV (the beta coronavirus that causes severe acute respiratory syndrome, or SARS), and SARS-COV-2 (the coronavirus that causes COVID-19). Viral proteins involved in recognition, attachment and invasion of human host cells from these other coronaviruses, such as coronavirus S proteins, may be substituted for the S or S1 protein of MERS-COV in the polypeptide according to the invention. In combination with a CD40 ligand, these polypeptides may provide substantial immunity against coronaviruses and reduce the severity of side-effects associated with vaccination, such as vaccine-induced inflammation or immunological hypersensitivity to an exogenous antigen.

Animal coronaviruses include Infectious bronchitis virus (IBV) which causes avian infectious bronchitis; Porcine coronavirus (transmissible gastroenteritis coronavirus of pigs, TGEV); Bovine coronavirus (BCV), responsible for severe profuse enteritis in of young calves; Feline coronavirus (FCOV) causes mild enteritis in cats as well as severe Feline infectious peritonitis (other variants of the same virus); two types of canine coronavirus (CCoV) (one causing enteritis, the other found in respiratory diseases); Turkey coronavirus (TCV) causes enteritis in turkeys; Ferret enteric coronavirus causes epizootic catarrhal enteritis in ferrets; Ferret systemic coronavirus causes FIP-like systemic syndrome in ferrets; Pantropic canine coronavirus; porcine epidemic diarrhea virus (PED or PEDV), has emerged around the world. Its economic importance is as yet unclear, but shows high mortality in piglets. In some embodiments, the invention is directed to immunogenic polypeptides containing a ligand targeting CD40 and an S1 protein analog from another coronavirus which replaces the MERS-CoV S1 determinants in a CD40-targeted MERS-COV S1 fusion proteins.

Other viral antigen or fragment thereof, or variant thereof include but are not limited to a virus from one of the following families: Adenoviridae, Arenaviridae, Bunyaviridae, Caliciviridae, Coronaviridae, Filoviridae, Hepadnaviridae, Herpesviridae, Orthomyxoviridae, Papovaviridae, Paramyxoviridae, Parvoviridae, Picornaviridae, Poxviridae, Reoviridae, Retroviridae, Rhabdoviridae, or Togaviridae. The viral antigen can be from human papillomoa virus (HPV), human immunodeficiency virus (HIV), polio virus, hepatitis B virus, hepatitis C virus, smallpox virus (Variola major and minor), vaccinia virus, influenza virus, rhinoviruses, dengue fever virus, equine encephalitis viruses, rubella virus, yellow fever virus, Norwalk virus, hepatitis A virus, human T-cell leukemia virus (HTLV-I), hairy cell leukemia virus (HTLV-II), California encephalitis virus, Hanta virus (hemorrhagic fever), rabies virus, Ebola fever virus, Marburg virus, measles virus, mumps virus, respiratory syncytial virus (RSV), herpes simplex 1, herpes simplex 2, varicella-zoster virus, cytomegalovirus (CMV), Epstein-Barr virus (EBV), flavivirus, foot and mouth disease virus, chikungunya virus, lassa virus, arenavirus, Nipah virus, Lassa virus or cancer causing virus.

Influenza virus strains for use in vaccines change from season to season. In the current inter-pandemic period, vaccines typically include two influenza A strains (H1N1 and H3N2) and one influenza B strain, and trivalent vaccines are typical. The invention may also use viruses from pandemic strains (i.e. strains to which the vaccine recipient and the general human population are immunologically naive), such as H2, H5, H7 or H9 subtype strains (in particular of influenza A virus), and influenza vaccines for pandemic strains may be monovalent or may be based on a normal trivalent vaccine supplemented by a pandemic strain. Depending on the season and on the nature of the antigen included in the vaccine, however, the invention may protect against one or more of influenza A virus hemagglutinin subtypes H1, H2, H3, H4, H5, H6, H7, H8, H9, H10, H11, H12, H13, H14, H15 or H16. The invention may protect against one or more of influenza A virus NA subtypes N1, N2, N3, N4, N5, N6, N7, N8 or N9.

As well as being suitable for immunizing against inter-pandemic strains, the adjuvanted compositions of the invention are useful for immunizing against pandemic strains. The characteristics of an influenza strain that give it the potential to cause a pandemic outbreak are:
(a) it contains a new hemagglutinin compared to the hemagglutinins in currently-circulating human strains, i.e. one that has not been evident in the human population for over a decade (e.g. H2), or has not previously been seen at all in the human population (e.g. H5, H6 or H9, that have generally been found only in bird populations), such that the human population will be immunologically naive to the strain's hemagglutinin; (b) it is capable of being transmitted horizontally in the human population; and (c) it is pathogenic to humans. A virus with H5 haemagglutinin type is preferred for immunising against pandemic influenza, such as a H5N1 strain. Other possible strains include H5N3, H9N2, H2N2, H7N1 and H7N7, and any other emerging potentially pandemic strains. Within the H5 subtype, a virus may fall into HA clade 1, HA clade 1′, HA clade 2 or HA clade 3, with clades 1 and 3 being particularly relevant.

Other strains that can usefully be included in the compositions are strains which are resistant to antiviral therapy (e.g. resistant to oseltamivir and/or zanamivir), including resistant pandemic strains.

Compositions of the invention may include antigen(s) from one or more (e.g. 1, 2, 3, 4 or more) influenza virus strains, including influenza A virus and/or influenza B virus. Monovalent vaccines are not preferred, and where a vaccine includes more than one strain of influenza, the different strains are typically grown separately and are mixed after the viruses have been harvested and antigens have been prepared. Thus a process of the invention may include the step of mixing antigens from more than one influenza strain. A trivalent vaccine is preferred, including two influenza A virus strains and one influenza B virus strain.

In some embodiments of the invention, the compositions may include antigen from a single influenza A strain. In some embodiments, the compositions may include antigen from two influenza A strains, provided that these two strains are not H1N1 and H3N2. In some embodiments, the compositions may include antigen from more than two influenza A strains.

The influenza virus may be a reassortant strain, and may have been obtained by reverse genetics techniques. Reverse genetics techniques[e.g. 24-28] allow influenza viruses with desired genome segments to be prepared in vitro using plasmids. Typically, it involves expressing (a) DNA molecules that encode desired viral RNA molecules e.g. from poll promoters, and (b) DNA molecules that encode viral proteins e.g. from polII promoters, such that expression of both types of DNA in a cell leads to assembly of a complete intact infectious virion. The DNA preferably provides all of the viral RNA and proteins, but it is also possible to use a helper virus to provide some of the RNA and proteins. Plasmid-based methods using separate plasmids for producing each viral RNA are preferred [29-31], and these methods will also involve the use of plasmids to express all or some (e.g. just the PB1, PB2, PA and NP proteins) of the viral proteins, with 12 plasmids being used in some methods.

As well as including diphtheria toxoid, a tetanus toxoid, a pertussis toxoid and/or poliovirus antigens, immunogenic compositions of the invention may include antigens from further pathogens. For example, these antigens may be HBsAg, conjugated Hib capsular saccharide, conjugated N.meningitidis capsular saccharide (one or more of serogroups A, C, W135 and/or Y) or conjugated S. pnetimonide capsular saccharide. For example, any of the suitable antigen components of PEDIARIX, MENVEO, MENACTRA, NIMENRIX, PREVNAR, or SYNFLORIX can be used.

Antigens are or derive from cellular pathogens, particularly from bacteria or fungi such as Actinobaccilus pleuropneumoniae, Pasteurella multocida, Streptococcus pneumonia, Streptococcus pyogenes, E. coli, Salmonella, Shigella, Yersinia, Campylobacter, Clostridium, Vibrio and Giardia, Entamoeba, and Cryptosporidium.

In a particular embodiment, the at least one antigen comprises a bacterial cell, preferably a live, attenuated, or inactivated bacterium. Within the context of the present invention the bacterial cell can comprise whole cells, cell sub-fractions or debris or pellets thereof.

In an embodiment, the bacterial cell is a salmonella bacterium, preferably selected from strains of Salmonella enteritidis, Salmonella kentucky, Salmonella typhimurium, Salmonella heidelberg, or a combination thereof. More particularly, the antigen comprises a combination of several different bacterial cells, more preferably of different strains of Salmonella, and/or sub-fractions thereof. In a preferred embodiment, the antigen comprises at least two different Salmonella cells selected from Salmonella enteritidis, Salmonella typhimurium, and Salmonella kentucky.

Non-limiting examples of the bacterial antigens include an inactivated or attenuated preparation(s) of at least one bacterium selected from the group consisting of Haemophilus influenzae, Streptococcus pneumoniae, Bordetella pertussis, tetanus bacilli, Corynebacterium diphtheriae, Tubercle bacilli, Escherichia coli such as enterohemorrhagic Escherichia coli, Vibrio cholerae, salmonellae, and methicillin-resistant Staphylococcus aureus or a part or a component thereof.

Non-limiting examples of the allergens include pollen (cedar pollen, Poaceae pollen, Compositae pollen, and the like), fungi, insects, foods (soybean, egg, milk, and the like), and drugs (penicillin and the like).

According to a further embodiment of the invention the antigen is originating from a pathogen selected from the group consisting of bacteria as Chlamydia, Clostridia, Brucella, Yersinia or virus, specifically selected from the group consisting of outer membrane protein 2 (OMP2), class I accessible protein 1 (Cap1), cysteine-rich protein A (CrpA), Chlamydia polymorphic membrane proteins (Pmps), specifically PmpA to PmpI, Chlamydia heat shock protein 60 (HSP60), Chlamydia heat shock protein 10 (HSP10), Chlamydia protease-like activity factor (CPAF), Yersinia pseudotuberculosis (YopD) or a homolog thereof, enolase, arginine binding protein (ArtJ), V-type ATP synthase subunit A (AtpA), peptidyl-prolyl cis-trans isomerase (Mip), glycogen synthase (GIgA), iron binding protein (YtgA), Vtype ATP synthase subunit E (AtpE), type III secretion chaperone (SycD), type III secretion proteins SctC or SctJ, tetanus toxoid, herpes simplex virus, varicella zoster virus or any combinations, fragments or derivatives thereof.

Cancer vaccines are designed to treat cancers by boosting the body's natural ability to protect itself, through the immune system. It has always represented a very attractive therapeutic approach, especially in light of the many shortcomings of conventional surgery, radiation and chemotherapies in the management of cancer. However, due to the low immunogenicity of the cancer carbohydrate antigen and the fact that many synthetic vaccines induce mainly IgM and to a lesser extent IgG antibody, the effectiveness of such cancer vaccine is still low. Various approaches have been explored, such as the use of an adjuvant, to aid immune recognition and activation.

Of the tumor associated glycans reported, the glycolipid antigen Globo H (Fuc.alpha.1.fwdarw.2 Gal.beta.1.fwdarw.3 GalNAc .beta.1.fwdarw.3 Gal.alpha.1.fwdarw.4 Gal.beta. 1.fwdarw.4 Glc) was first isolated and identified in 1984 by Hakomori et al. from breast cancer MCF-7 cells. (Bremer E G, et al. (1984) J Biol Chem 259:14773-14777.) Further studies with anti-Globo H monoclonal antibodies showed that Globo H was present on many other cancers, including prostate, gastric, pancreatic, lung, ovarian and colon cancers and only minimal expression on luminal surface of normal secretory tissue which is not readily accessible to immune system. (Ragupathi G, et al. (1997) Angew Chem Int Ed 36:125-128.) In addition, it has been established that the serum of breast cancer patient contains high level of anti-Globo H antibody. (Gilewski T et al. (2001) Proc Natl Acad Sci USA 98:3270-3275; Huang C—Y, et al. (2006) Proc Natl Acad Sci USA 103:15-20; Wang C-C, et al. (2008) Proc Natl Acad Sci USA 105 (33): 11661-11666) and patients with Globo H-positive tumors showed a shorter survival in comparison to patients with Globo H-negative tumors. (Chang, Y-J, et al. (2007) Proc Natl Acad Sci USA 104 (25): 10299-10304.) These findings render Globo H, a hexasaccharide epitope, an attractive tumor marker and a feasible target for cancer vaccine development.

Other vaccines and antigens contained therein that can be used in the compositions, combinations and methods described herein include:

| | |
|---|---|
| Adenovirus | Pertussis |
| Anthrax | DTaP (Daptacel, Infanrix) |
| AVA (BioThrax) | Tdap (Adacel, Boostrix) |
| Cholera | DTaP-IPV (Kinrix, Quadracel) |
| Vaxchora | DTaP-HepB-IPV (Pediarix) |
| Diphtheria | DTaP-IPV/Hib (Pentacel) |
| DTaP (Daptacel, Infanrix) | Pneumococcal |
| Td (Tenivac, generic) | PCV13 (Prevnar13) |
| DT (-generic-) | PPSV23 (Pneumovax 23) |
| Tdap (Adacel, Boostrix) | Pollo |
| DTaP-IPV (Kinrix, Quadracel) | Polio (Ipol) |
| DTaP-HepB-IPV (Pediarix) | DTaP-IPV (Kinrix, Quadracel) |
| DTaP-IPV/Hib (Pentacel) | DTaP-HepB-IPV (Pediarix) |
| Hepatitis A | DTaP-IPV/Hib (Pentacel) |
| HepA (Havrix, Vaqts) | Rabies |
| HepA-HepB (Twinrix) | Rabies (Imovax Rabies, RabAvert) |
| Hepatitis B | Rotavirus |
| HepB (Engerix-B, Recombivex HB, Heplisav-B) | RV1 (Rotarix) |
| DTaP-HepB-IPV (Pediarix) | RV5 (RotaTeq) |
| HepA-HepB (Twinrix) | Rubella |
| Haemophilus influenzae type b (Hib) | MMR (M-M-R II) |
| Hib (ActHIB, PedvaxHIB, Hiberix) | MMRV (ProQuad) |
| DTaP-IPV/Hib (Pentacel) | Shingles |
| Human Papillomavirus (HPV) | RZV (Shingrix) |
| HPV9 (Gardasil 9) (For scientific papers, the | Smallpox |
| preferred abbreviation is 9vHPV) | Vaccinia (ACAM2000): |
| Seasonal Influenza (Flu) only | Tetanus |
| IIV* (Afluria, Fluad, Flublok, Flucelvax, | DTaP (Daptacel, Infanrix) |
| FluLaval, Fluarix, Fluvirin, Fluzone, | Td (Tenivac, generic) |
| Fluzone High-Dose, Fluzone Intradermal) | DT (-generic-) |
| *There are various acronyms for inactivated flu | Tdap (Adacel, Boostrix) |
| vaccines-IIV3, IIVA, RIV3, RIV4 and cellV4. | DTaP-IPV (Kinrix, Quadracel) |
| LAIV (FluMist) | DTaP-HepB-IPV (Pediarix) |
| Japanese Encephalitis | DTaP-IPV/Hib (Pentacel) |
| JE (Ixiaro) | Tuberculosis |
| Measles | Typhoid Fever |
| MMR (M-M-R II) | Typhoid Oral (Vivotif) |
| MMRV (ProQuad) | Typhoid Polysaccharide (Typhim Vi) |
| Meningococcal | Varicella |
| MenACWY (Menactra, Menveo) | VAR (Varivax) |
| MenB (Bexsero, Trumenba) | MMRV (ProQuad): |
| Mumps | Yellow Fever |
| MMR (M-M-R II) | YF (YF-Vax) |
| MMRV (ProQuad) | |

### Other Adjuvants and Ingredients

Other Adjuvants can be used in conjunction with the chemical entities described herein and having, e.g., formula I include aluminum hydroxide and aluminum phosphate, saponins e.g., Quil A, QS-21 (Cambridge Biotech Inc., Cambridge Mass.), GPI-0100 (Galenica Pharmaceuticals, Inc., Birmingham, Ala.), water-in-oil emulsion, oil-in-water emulsion, water-in-oil-in-water emulsion. The emulsion can be based in particular on light liquid paraffin oil (European Pharmacopea type); isoprenoid oil such as squalane or squalene; oil resulting from the oligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers are preferably nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the Pluronic products, especially L121. See Hunter et al., The Theory and Practical Application of Adjuvants (Ed.Stewart-Tull, D. E. S.). JohnWiley and Sons, NY, pp 51-94 (1995) and Todd et al., Vaccine 15:564-570 (1997). For example, it is possible to use the SPT emulsion described on page 147 of “Vaccine Design, The Subunit and Adjuvant Approach” edited by M. Powell and M. Newman, Plenum Press, 1995, and the emulsion MF59 described on page 183 of this same book. Further suitable adjuvants include, but are not limited to, the RIBI adjuvant system (Ribi Inc.), Block co-polymer (Cytx, Atlanta GA), SAF-M (Chiron, Emeryville Calif.), monophosphoryl lipid A, Avridine lipid-amine adjuvant, heat-labile enterotoxin from E. coli (recombinant or otherwise), cholera toxin, IMS 1314 or muramyl dipeptide among many others. Among the copolymers of maleic anhydride and alkenyl derivative, the copolymers EMA (Monsanto), which are copolymers of maleic anhydride and ethylene, are included. The dissolution of these polymers in water leads to an acid solution that will be neutralized, preferably to physiological pH, in order to give the adjuvant solution into which the immunogenic, immunological or vaccine composition itself will be incorporated.

In one aspect of the present invention the pharmaceutical-acceptable carrier is an adjuvant selected from the group consisting of aluminum hydroxide, aluminum phosphate, saponins, water-in-oil emulsion, oil-in-water emulsion, water-in-oil-in-water emulsion, polymers of acrylic or methacrylic acid, copolymers of maleic anhydride and alkenyl derivative, the RIBI adjuvant system, Block co-polymer, SAF-M, monophosphoryl lipid A, Avridine lipid-amine, heat-labile enterotoxin from E. coli (recombinant or otherwise), cholera toxin, IMS 1314, muramyl dipeptide, and combinations thereof. Thus, according to one aspect, the present application provides an immunogenic composition comprising a) one or more antigens of M. hyorhinis; and one or more antigens of M. hyosynoviae; and b) a pharmaceutically acceptable carrier, wherein the pharmaceutical-acceptable carrier is an adjuvant selected from the group consisting of aluminum hydroxide, aluminum phosphate, saponins, water-in-oil emulsion, oil-in-water emulsion, water-in-oil-in-water emulsion, polymers of acrylic or methacrylic acid, copolymers of maleic anhydride and alkenyl derivative, the RIBI adjuvant system, Block co-polymer, SAF-M, monophosphoryl lipid A, Avridine lipid-amine, heat-labile enterotoxin from E. coli (recombinant or otherwise), cholera toxin, IMS 1314, muramyl dipeptide, and combinations thereof. Such vaccine can also comprise one or more antigens of M. hyopneumoniae. Furthermore, one or more of the mycoplasma antigens of such mycoplasma species can be provided as whole inactivated bacterin as described herein above.

A further example of an adjuvant is a compound chosen from the polymers of acrylic or methacrylic acid and the copolymers of maleic anhydride and alkenyl derivative. Advantageous adjuvant compounds are the polymers of acrylic or methacrylic acid which are cross-linked, especially with polyalkenyl ethers of sugars or polyalcohols. These compounds are known by the term carbomer (Pharmeuropa Vol. 8, No. 2, June 1996). Persons skilled in the art can also refer to U.S. Pat. No. 2,909,462 which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms, e.g. vinyls, allyls and other ethylenically unsaturated groups. The unsaturated radicals may themselves contain other substituents, such as methyl. The products sold under the name CARBOPOL.RTM.; (BF Goodrich, Ohio, USA) are particularly appropriate. They are polymers of acrylic acid cross-linked with polyalkenyl ethers or divinyl glycol or cross-linked with an allyl sucrose or with allyl pentaerythritol. Among them, there may be mentioned CARBOPOL.RTM. 974P, 934P and 971P. Most preferred is the use of CARBOPOL.RTM. 971P.

Surfactant(s) are typically selected, or combined, or used under conditions providing a proper Hydrophilic-Lipophilic Balance (HLB) to the formulation. The HLB of a surfactant or of a combination of surfactants is a measure of the degree to which it is hydrophilic or lipophilic, determined by calculating values for the different regions of the molecules as described by Griffin (Journal of the Society of Cosmetic Chemists, 1949, 1 (5), 311-26 and Journal of the Society of Cosmetic Chemists, 1954, 5 (4), 249-56).

Examples of surfactant used in emulsion vaccines include, without limitation, sorbitan monooleate (Span 80), polyoxyethylene sorbitan monooleate (Tween 80), sorbitan sesquioleate (Span 83), Lecithin, and mannide monooleate, or mixtures thereof.

Vaccines and compositions of the invention optionally further comprise one or several salts. The addition of a salt can inhibit osmosis of water into the oily particles and further stabilize the oily particles. Examples of such salts include, without limitation, sodium chloride, magnesium chloride, sodium sulfate or magnesium sulfate. In a particular embodiment, the salt is sodium chloride.

The compositions of the invention may further comprise one or more preservatives that are acceptable in the veterinary field. Without limitation, examples of suitable preservatives include: acids, such as benzoic acid, sorbic acids and sodium or potassium salts thereof; esters, such as methylparaben, ethylparaben and propylaparaben; alcohols, such as chlorobutanol, benzyl alcohol, phenyl ethyl alcohol, phenoxyethanol, phenols such as chlorocresol and o-phenyl phenol; mercurial compounds such as thimerosal, nitromersol, phenylmercuric nitrate and phenylmercuric acetate; quaternary ammonium compounds such as benalkonium chloride and cetyl pyridium chloride. In a preferred embodiment, the preservative is a thimerosal solution, and typically a 10% thimerosal solution.

### Methods of Treatment

In some embodiments, this disclosure provides methods for promoting an immune responses in a subject in need thereof, comprising administering to the subject a therapeutically effective amount of a composition comprising chemical entities described herein (e.g., compounds of Formulae disclosed herein), or pharmaceutically acceptable salts, hydrates, solvates, or prodrugs thereof. In some embodiments, the compound is selected from the group consisting of: UDPS-Heptose, CDPS-Heptose, and ADPS-Heptose.

Peritumoral injection of chemical entities described herein (e.g., UDPS-Heptose) rejected tumor growth in several mouse tumor models. Chemical entities described herein (e.g., UDPS-Heptose) have also exhibited systemic immune promotion function. ALPK1 is widely expressed in human (https://www.proteinatlas.org/ENSG00000073331-ALPK1/tissue). As such, without wishing to be bound by theory, it is believed that through activation of ALPK1, chemical entities disclosed herein can have beneficial effects in the treatment of multiple types of cancer.

Accordingly, in some embodiments, the present disclosure provides methods of using chemical entities described in e.g., compounds of Formulae disclosed herein, including UDPS-Heptose, ADPS-Heptose, and CDPS-Heptose (e.g., serving as ALPK1 agonists) to treat a cancer. The method comprising administering to a patient in need thereof a therapeutically effective amount of ALPK1 agonist, selected from the chemical entities described herein (e.g., compounds of Formulae disclosed herein), or a pharmaceutically acceptable salt or prodrug thereof. In some embodiments, the ALPK1 agonist is selected from the group consisting of UDPS-Heptose, ADPS-Heptose, and CDPS-Heptose.

Administration of the chemical entities disclosed herein (e.g., compounds of Formulae disclosed herein) or the pharmaceutically acceptable salts thereof can be via any of the accepted modes of administration, including, but not limited to, orally, subcutaneously, intravenously, intranasally, topically, transdermally, intraperitoneally, intramuscularly, intrapulmonarilly, vaginally, rectally, ontologically, neuro-otologically, intraocularly, subconjuctivally, via anterior eye chamber injection, intravitreally, intraperitoneally, intrathecally, intracystically, intrapleurally, via wound irrigation, intrabuccally, intra-abdominally, intra-articularly, intra-aurally, intrabronchially, intracapsularly, intrameningeally, via inhalation, via endotracheal or endobronchial instillation, via direct instillation into pulmonary cavities, intraspinally, intrasynovially, intrathoracically, via thoracostomy irrigation, epidurally, intratympanically, intracisternally, intravascularly, intraventricularly, intraosseously, via irrigation of infected bone, or via application as part of any admixture with a prosthetic devices. In some embodiments, the administration method includes oral or parenteral administration.

Provided herein are methods for treating cancer in a subject in need thereof, including administering to the subject a therapeutically effective amount of a chemical entity described herein (e.g., a compound of Formulae disclosed herein), or pharmaceutically acceptable salts, hydrates, solvates, or prodrugs thereof, in combination with one or more cancer immunotherapy agents/immune modulators. The cancer immunotherapy agents used herein, are effective to enhance, stimulate, and/or up-regulate immune responses in a subject. Administration of a compound of the present disclosure with a cancer immunotherapy agent has can have a synergistic effect in cancer treatment.

In some embodiments, the immunotherapy agent is an agonist of a stimulatory (including a co-stimulatory) receptor or an antagonist of an inhibitory (including a co-inhibitory) signal on immune cells, including but not limited to T-cells, dendritic cells, and natural killer cells, both of which result in amplifying antigen-specific T cell responses (often referred to as immune checkpoint regulators).

In some embodiments, the immunotherapy agents include, but are not limited to, a small molecule drug, antibody, or other biologic molecules. In some embodiments, the biologic immunotherapy agents include, but are not limited to, cancer vaccines, antibodies, therapeutic engineered-immune cells. In some embodiments, the therapeutic engineered-immune cell is a chimeric antigen receptor T cell (CAR-T), a chimeric antigen receptor natural killer cell (CAR-NK), or a T cell receptor engineered-T cell (TCR-T). In some embodiments, the biologic immunotherapy agent is an antibody. In some embodiments, the antibody is a monoclonal antibody. In some embodiments, the monoclonal antibody is humanized.

In some embodiments, the antibody is an agonist of a stimulatory (including a co-stimulatory) ligand/receptor on immune cells. In some embodiments, the antibody is an antagonist of an inhibitory (including a co-inhibitory) ligand/receptor on immune cells.

In some embodiments, the stimulatory or inhibitory ligands/receptors include, but are not limited to, members of B7 family, which includes B7-1, B7-2, B7-H1 (PD-L1), B7-DC (PD-L2), B7-H2 (ICOS-L), B7-H3, B7-H4, B7-H5 (VISTA), B7-H6, and B7-H7.

In some embodiments, the stimulatory or inhibitory ligands/receptors include, but are not limited to, members of the TNF/TNF receptor family, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fnl4, TWEAK, BAFFR, EDAR, XEDAR, TACI, APRIL, BCMA, LIGHT, DCR3, HVEM, VEGI/TL1A, TRAMP/DR3, EDAR, EDA1, XEDAR, EDA2, TNF-R¹, Lymphotoxin a/TNFβ, TNFR2, TNFa, LTBR, Lymphotoxin a1β2, FAS, FASL, RELT, DR6, TROY, NGFR.

T cell responses can be stimulated by a combination of anti-CD40 antibodies described herein, e.g., 3C3 and 3G5, and one or more of an antagonist (inhibitor or blocking agent) of a protein that inhibits T cell activation (e.g., immune checkpoint inhibitors), such as CTLA-4, PD-1, PD-L1, PD-L2, and LAG-3, as described above, and any of the following proteins: TIM-3, Galectin 9, CEACAM-1, BTLA, CD69, Galectin-1, TIGIT, CD113, GPR56, VISTA, B7-H3, B7-H4, 2B4, CD48, GARP, PDIH, LAIR1, TIM-1, and TIM4-4, and/or one or more of an agonist of a protein that stimulates T cell activation, such as B7-1, B7-2, CD28, 4-1BB (CD137), 4-1BBL, ICOS, ICOS-L, OX40, OX40L, CD70, CD27, CD40, DR3 and CD28H.

In some embodiments, the inhibitory ligand/receptor is selected from PD-1, PD-L1, PD-L2, CTLA4, LAG-3, TIM-3, VISTA, and TIGIT. In some embodiments, the inhibitory ligand/receptor is selected from PD-1, PD-L1, and CTLA4. In certain embodiments, the inhibitory ligand/receptor is PD-1 or PD-L1.

In some embodiments, the stimulatory ligand/receptor is selected from B7-1, B7-2, CD28, 4-1BB (CD137), 4-1BBL, ICOS, ICOS-L, OX40, OX40L, GITR, GITRL, CD70, CD27, CD40, DR3 and CD28H. In certain embodiments, the stimulatory ligand/receptor is 4-1BB (CD137), 4-1BBL, OX40 or OX40L.

In some embodiments, the antibody is selected from the group consisting nivolumab, pembrolizumab, pidilizumab, cemiplimab, camrelizumab, tislelizumab, BMS-936559, atezolizumab, durvalumab, and avelumab. In some embodiments, the antibody is nivolumab or pembrolizumab. In some embodiments, the immune checkpoint is CTLA-4. In some embodiments, the antibody is ipilimumab. In some embodiments, the immune checkpoint is TIGIT.

In some embodiments, the immunotherapy agent is a therapeutic engineered-immune cell is a chimeric antigen receptor T-cell (CAR-T), a chimeric antigen receptor natural killer cell (CAR-NK), or a T-cell receptor engineered T-cell (TCR-T). In some embodiments, the CAR-T therapy is Kymriah (tisagenlecleucel), Yescarta (axicabtagene ciloleucel), or Tecartus (brexucabtagene autoleucel).

Exemplary immunothepray agents that modulate one of the above proteins and may be combined those described herein, for treating cancer, include: Yervoy™ (ipilimumab) or Tremelimumab (to CTLA-4), galiximab (to B7.1), BMS-936558/nivolumab (to PD-1), MK-3475/pembrolizumab (to PD-1), AMP224 (to B7DC), BMS-936559 (to B7-H1), MPDL3280A/atezolizumab (to B7-H1), MEDI-570 (to ICOS), AMG557 (to B7H2), MGA271 (to B7H3), IMP321 (to LAG-3), BMS-663513 (to CD137), PF-05082566 (to CD137), CDX-1127 (to CD27), anti-OX40 (Providence Health Services), huMAbOX40L (to OX40L), Atacicept (to TACI), CP-870893 (to CD40), Lucatumumab (to CD40), Dacetuzumab (to CD40), Muromonab-CD3 (to CD3), Ipilumumab (to CTLA-4).

In some embodiments, the compound is selected from the group consisting of a compound of Formulae disclosed herein), or pharmaceutically acceptable salts, hydrates, solvates, or prodrugs thereof. In some embodiments, the ALPK1 agonist is selected from the group consisting of: UDPS-Heptose, ADPS-Heptose, and CDPS-Heptose.

Accordingly, in some embodiments, types of cancer include, but are not limited to:

1) Breast cancers, including, for example ER breast cancer, ER-breast cancer, her2-breast cancer, her2 breast cancer, stromal tumors such as fibroadenomas, phyllodes tumors, and sarcomas, and epithelial tumors such as large duct papillomas; carcinomas of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma; and miscellaneous malignant neoplasms. Further examples of breast cancers can include luminal A, luminal B, basal A, basal B, and triple negative breast cancer, which is estrogen receptor negative (ER″), progesterone receptor negative, and her2 negative (her2″). In some embodiments, the breast cancer may have a high risk Oncotype score.

2) Cardiac cancers, including, for example sarcoma, e.g., angiosarcoma, fibrosarcoma, rhabdomyosarcoma, and liposarcoma; myxoma; rhabdomyoma; fibroma; lipoma and teratoma.

3) Lung cancers, including, for example, bronchogenic carcinoma, e.g., squamous cell, undifferentiated small cell, undifferentiated large cell, and adenocarcinoma; alveolar and bronchiolar carcinoma; bronchial adenoma; sarcoma; lymphoma; chondromatous hamartoma; and mesothelioma.

4) Gastrointestinal cancer, including, for example, cancers of the esophagus, e.g., squamous cell carcinoma, adenocarcinoma, leiomyosarcoma, and lymphoma; cancers of the stomach, e.g., carcinoma, lymphoma, and leiomyosarcoma; cancers of the pancreas, e.g., ductal adenocarcinoma, insulinoma, glucagonoma, gastrinoma, carcinoid tumors, and vipoma; cancers of the small bowel, e.g., adenocarcinoma, lymphoma, carcinoid tumors, Kaposi's sarcoma, leiomyoma, hemangioma, lipoma, neurofibroma, and fibroma; cancers of the large bowel, e.g., adenocarcinoma, tubular adenoma, villous adenoma, hamartoma, and leiomyoma.

5) Genitourinary tract cancers, including, for example, cancers of the kidney, e.g., adenocarcinoma, Wilm's tumor (nephroblastoma), lymphoma, and leukemia; cancers of the bladder and urethra, e.g., squamous cell carcinoma, transitional cell carcinoma, and adenocarcinoma; cancers of the prostate, e.g., adenocarcinoma, and sarcoma; cancer of the testis, e.g., seminoma, teratoma, embryonal carcinoma, teratocarcinoma, choriocarcinoma, sarcoma, interstitial cell carcinoma, fibroma, fibroadenoma, adenomatoid tumors, and lipoma.

6) Liver cancers, including, for example, hepatoma, e.g., hepatocellular carcinoma; cholangiocarcinoma; hepatoblastoma; angiosarcoma; hepatocellular adenoma; and hemangioma.

7) Bone cancers, including, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors.

8) Nervous system cancers, including, for example, cancers of the skull, e.g., osteoma, hemangioma, granuloma, xanthoma, and osteitis deformans; cancers of the meninges, e.g., meningioma, meningiosarcoma, and gliomatosis; cancers of the brain, e.g., astrocytoma, medulloblastoma, glioma, ependymoma, germinoma (pinealoma), glioblastoma multiform, oligodendroglioma, schwannoma, retinoblastoma, and congenital tumors; and cancers of the spinal cord, e.g., neurofibroma, meningioma, glioma, and sarcoma.

9) Gynecological cancers, including, for example, cancers of the uterus, e.g., endometrial carcinoma; cancers of the cervix, e.g., cervical carcinoma, and pre tumor cervical dysplasia; cancers of the ovaries, e.g., ovarian carcinoma, including serous cystadenocarcinoma, mucinous cystadenocarcinoma, unclassified carcinoma, granulosa theca cell tumors, Sertoli Leydig cell tumors, dysgerminoma, and malignant teratoma; cancers of the vulva, e.g., squamous cell carcinoma, intraepithelial carcinoma, adenocarcinoma, fibrosarcoma, and melanoma; cancers of the vagina, e.g., clear cell carcinoma, squamous cell carcinoma, botryoid sarcoma, and embryonal rhabdomyosarcoma; and cancers of the fallopian tubes, e.g., carcinoma.

10) Hematologic cancers, including, for example, cancers of the blood, e.g., acute myeloid leukemia, chronic myeloid leukemia, acute lymphoblastic leukemia, chronic lymphocytic leukemia, myeloproliferative diseases, multiple myeloma, and myelodysplastic syndrome, Hodgkin's lymphoma, non-Hodgkin's lymphoma (malignant lymphoma) and Waldenström's macroglobulinemia.

11) Skin cancers and skin disorders, including, for example, malignant melanoma and metastatic melanoma, basal cell carcinoma, squamous cell carcinoma, Kaposi's sarcoma, moles dysplastic nevi, lipoma, angioma, dermatofibroma, keloids, and scleroderma.

12) Adrenal gland cancers, including, for example, neuroblastoma.

Cancers may also occur, as in leukemia, as a diffuse tissue. Thus, the term “tumor cell,” as provided herein, includes a cell afflicted by any one of the above identified disorders.

In certain embodiments, the cancer is metastatic. In certain embodiments, the cancer is refractory.

In certain embodiments, the cancer is selected from the group consisting of neuroblastoma, intestinal carcinoma such as rectal carcinoma, colon carcinomas, familiar adenomatous polyposis carcinoma and hereditary non-polyposis colorectal cancer, esophageal carcinoma, labial carcinoma, larynx carcinoma, nasopharyngeal cancers, oral cavity cancers, salivary gland carcinoma, peritoneal cancers, soft tissue sarcoma, urothelial cancers, sweat gland carcinoma, gastric carcinoma, adenocarcinoma, medullary thyroid carcinoma, papillary thyroid carcinoma, renal carcinoma, kidney parenchymal carcinoma, ovarian carcinoma, cervical carcinoma, uterine corpus carcinoma, endometrial carcinoma, pancreatic carcinoma, hepatocellular cancer, prostate carcinoma, testis carcinoma, breast cancers including HER2 Negative, urinary carcinoma, melanoma, brain tumors such as glioblastoma, astrocytoma, meningioma, medulloblastoma and peripheral neuroectodermal tumors, Hodgkin's lymphoma, non-Hodgkin's lymphoma, Burkitt lymphoma, acute lymphatic leukemia (ALL), chronic lymphatic leukemia (CLL), acute myeloid leukemia (AML), chronic myeloid leukemia (CML), adult T-cell leukemia lymphoma, diffuse large B-cell lymphoma (DLBCL), hepatocellular carcinoma, multiple myeloma, seminoma, osteosarcoma, chondrosarcoma, anal canal cancers, adrenal cortex carcinoma, chordoma, fallopian tube cancer, gastrointestinal stromal tumors, myeloproliferative diseases, mesothelioma, biliary tract cancers, Ewing sarcoma and other rare tumor types.

In certain embodiments, the cancer is selected from the group consisting of: brain cancers, skin cancers, bladder cancers, ovarian cancers, breast cancers, gastric cancers, pancreatic cancers, hepatocellular cancer, prostate cancers, colorectal cancers, blood cancers, lung cancers and bone cancers. In certain embodiments, the cancer is selected from the following group: small cell lung cancer, non-small cell lung cancer, colorectal cancer, melanoma, renal cell carcinoma, head and neck cancer, Hodgkin's lymphoma or bladder cancer.

In certain embodiments, the methods described herein can further include administering one or more additional cancer therapies. The one or more additional cancer therapies can include, without limitation, surgery, radiotherapy, chemotherapy, toxin therapy, immunotherapy, cryotherapy, cancer vaccines (e.g., HPV vaccine, hepatitis B vaccine, Oncophage, Provenge) and gene therapy, as well as combinations thereof. Immunotherapy, including, without limitation, adoptive cell therapy, the derivation of stem cells and/or dendritic cells, blood transfusions, lavages, and/or other treatments, including, without limitation, freezing a tumor.

In some embodiments, the present disclosure provides methods for the treatment of immune or inflammatory related diseases in a subject in need thereof, comprising administering to the subject a therapeutically effective amount of a composition comprising chemical entities described herein (e.g., compounds of Formulae disclosed herein), or pharmaceutically acceptable salts, hydrates, solvates, or prodrugs thereof (e.g., UDPS-Heptose, ADPS-Heptose, CDPS-Heptose, TDPS-Heptose or derivatives thereof). In some embodiments, the compound is selected from the group consisting of UDPS-Heptose, CDPS-Heptose, and ADPS-Heptose.

Non-limiting examples of immune or inflammatory related disease include rheumatoid arthritis, systemic lupus erythematosus, multiple sclerosis, inflammatory bowel diseases (IBDs) comprising Crohn disease (CD) and ulcerative colitis (UC), which are chronic inflammatory conditions with polygenic susceptibility. In certain embodiments, the disease is an inflammatory bowel disease (IBD). In certain embodiments, the disease is Crohn's disease, autoimmune colitis, iatrogenic autoimmune colitis, ulcerative colitis, colitis induced by one or more chemotherapeutic agents, colitis induced by treatment with adoptive cell therapy, colitis associated by one or more alloimmune diseases (such as graft-vs-host disease, e.g., acute graft vs. host disease and chronic graft vs. host disease), radiation enteritis, collagenous colitis, lymphocytic colitis, microscopic colitis, and radiation enteritis. In certain of these embodiments, the condition is alloimmune disease (such as graft-vs-host disease, e.g., acute graft vs. host disease and chronic graft vs. host disease), celiac disease, irritable bowel syndrome, rheumatoid arthritis, lupus, scleroderma, psoriasis, cutaneous T-cell lymphoma, uveitis, and mucositis (e.g., oral mucositis, esophageal mucositis or intestinal mucositis).

In certain embodiments, the immune or inflammatory related disease is an autoimmune disease. Non-limiting examples of autoimmune diseases include: arthritis (including rheumatoid arthritis, juvenile rheumatoid arthritis, osteoarthritis, psoriatic arthritis), multiple sclerosis, myasthenia gravis, systemic lupus erythematosis, autoimmune thyroiditis (e.g., Hashimoto's thyroiditis), dermatitis (including atopic dermatitis and eczematous dermatitis), psoriasis, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves ophthalmopathy, sarcoidosis, primary biliary cirrhosis, uveitis posterior, and interstitial lung fibrosis.

In some embodiments, this disclosure provides methods of promoting systemic immune responses in a subject in need thereof comprising administering to the subject an effective amount of a chemical entity described herein (e.g., compounds of Formulae disclosed herein), or pharmaceutically acceptable salts, hydrates, solvates, or prodrugs thereof (e.g., UDPS-Heptose, ADPS-Heptose, CDPS-Heptose, TDPS-Heptose or derivatives thereof).

In some embodiments, this disclosure provides methods of inducing cytokine production and/or NF-κB pathway activation in a subject in need thereof comprising administering to the subject an effective amount of a chemical entity described herein (e.g., compounds of Formulae disclosed herein), or pharmaceutically acceptable salts, hydrates, solvates, or prodrugs thereof (e.g., UDPS-Heptose, ADPS-Heptose, CDPS-Heptose, TDPS-Heptose or derivatives thereof).

Accordingly, in some embodiments, this disclosure provides methods of treating a disease or disorder associated with NF-κB, p38, and/or JNK cell signaling pathways in a subject in need thereof. In certain embodiments, repressed or impaired NF-κB pathway, p38, and JNK cell signaling contributes to the pathology and/or symptoms and/or progression of the disease.

In certain embodiments, the disease or disorder is selected from the group consisting of: autoimmune diseases such as chronic rheumatism, osteoarthritis, systematic lupus erythematosus, systematic scleroderma, polymyositis, Sjogren's syndrome, vasculitis syndrome, antiphospholipid syndrome, Still's disease, Behcet's disease, periarteritis nodosa, ulcerative colitis, Crohn's disease, active chronic hepatitis, glomerulonephritis, and chronic nephritis, chronic pancreatitis, gout, atherosclerosis, multiple sclerosis, arteriosclerosis, endothelial hypertrophy, psoriasis, psoriatic arthritis, contact dermatitis, atopic dermatitis, allergic disease such as pollinosis, asthma, bronchitis, interstitial pneumonia, lung disease involving granuloma, chronic obstructive lung disease, chronic pulmonary thromboembolism, inflammatory colitis, insulin resistance, obesity, diabetes and its complications (nephropathy, retinopathy, neurosis, hyperinsulinemia, arteriosclerosis, hypercentiona, peripheral vessel obstruction, etc.) diseases involving abnormal vascular proliferation such as hyperlipemia, retinopathy, and pneumonia, Alzheimer's disease, encephalomyelitis, acute hepatitis, chronic hepatitis, drug induced toxic hepatopathy, alcoholic hepatitis, viral hepatitis, icterus, cirrhosis, hepatic insufficiency, atrial myxoma, Caslemann's syndrome, mesangial nephritis, kidney cancer, lung cancer, liver cancer, breast cancer, uterine cancer, pancreatic cancer, other solid cancer, sarcoma, osteosarcoma, metastatic invasion of cancer, carceration of inflammatory focus, cancerous cachexia, metastasis of cancer, leukemia, such as acute myeloblastic leukemia, multiple myeloma, Lennert's lymphoma, malignant lymphoma, development of carcinostatic resistance of cancer, carciration of foci such as viral hepatitis and cirrhosis, carciration from polyp of colon, brain tumor, nervous tumor, endotoxic shock, sepsis, cytome, galoviral pneumonia, cytomegaloviral retinopathy, adenoviral cold, adenoviral pool fever, adenoviral ophthalmia, conjunctivitis, AIDS, uveitis, diseases or complications provoked by infections of other bacteria, viruses, and mycetes, complications after surgery such as generalized inflammatory symptoms, restenosis after percutaneous tubal coronary artery plastic surgery, reperfusion disorders after vascular occulusion opening such as ischemia reperfusion disorders, organ transplantation rejection and perfusion disorders of heart, liver, kidney, or the like, itch, anorexia, malaise, and chronic fatigue syndrome.

In certain embodiments, the disease or disorder is selected from the group consisting of tuberculosis, meningitis, pneumonia, ulcer, sepsis, rhinitis, asthma, allergy, COPD, inflammatory bowel disease, arthritis, obesity, radiation-induced inflammation, psoriasis, atopic dermatitis, non-alcoholic steatohepatitis (NASH), Alzheimer's disease, systemic lupus, erythematosus (SLE), autoimmune thyroiditis (Grave's disease), multiple sclerosis, ankylosing spondylitis bullous diseases, actinic keratoses, ulcerative colitis, Crohn's disease, alopecia areata, and diseases and disorders caused by the hepatitis C virus (HCV), the hepatitis B virus (HBV), or the human immunodeficiency virus (HIV).

In some embodiments, this disclosure provides methods of treatment of a disease in which repressed or impaired ALPK1 signaling contributes to the pathology and/or symptoms and/or progression of the disease comprising administering to a subject in need of such treatment an effective amount of a chemical entity described herein (e.g., compounds of Formulae disclosed herein), or pharmaceutically acceptable salts, hydrates, solvates, or prodrugs thereof (e.g., UDPS-Heptose, ADPS-Heptose, CDPS-Heptose, TDPS-Heptose or derivatives thereof). Non-limiting examples of the diseases include cancers or immune or inflammatory related diseases as described anywhere herein.

In some embodiments, this disclosure provides methods of treatment comprising administering to a subject having a disease in which repressed or impaired ALPK1 signaling contributes to the pathology and/or symptoms and/or progression of the disease an effective amount of a chemical entity described herein (e.g., compounds of Formulae disclosed herein), or pharmaceutically acceptable salts, hydrates, solvates, or prodrugs thereof (e.g., UDPS-Heptose, ADPS-Heptose, CDPS-Heptose, TDPS-Heptose or derivatives thereof). Non-limiting examples of the diseases include cancers or immune or inflammatory related diseases as described anywhere herein.

In some embodiments, this disclosure provides methods of treatment comprising administering to a subject a chemical entity described (e.g., compounds of Formulae disclosed herein), or pharmaceutically acceptable salts, hydrates, solvates, or prodrugs thereof (e.g., UDPS-Heptose, ADPS-Heptose, CDPS-Heptose, TDPS-Heptose or derivatives thereof), wherein the chemical entity is administered in an amount effective to treat a disease in which repressed or impaired ALPK1 signaling contributes to the pathology and/or symptoms and/or progression of the disease, thereby treating the disease. Non-limiting examples of the diseases include cancers or immune or inflammatory related diseases as described anywhere herein.

### Enhancing the Efficacy of a Vaccine

In another aspect, the present disclosure provides methods for enhancing the efficacy of a vaccine, comprising administering a therapeutically effective amount of chemical entities described herein (e.g., compounds of Formulae disclosed herein), or pharmaceutically acceptable salts, hydrates, solvates, or prodrugs thereof. In some embodiments, the compound is selected from the group consisting of: UDPS-Heptose, CDPS-Heptose, and ADPS-Heptose. In some embodiments, the vaccines are cancer vaccines. In some embodiments, the vaccines are bacterial vaccines. In some embodiments, the vaccines are viral vaccines. In some embodiments, the vaccines are parasite vaccines.

Also provided herein are methods of enhancing innate immunity in a subject in need thereof, including administering to the subject a therapeutically effective amount of chemical entities described herein (e.g., compounds Formulae disclosed herein), or pharmaceutically acceptable salts, hydrates, solvates, or prodrugs thereof. In some embodiments, the compound is selected from the group consisting of: UDPS-Heptose, CDPS-Heptose, and ADPS-Heptose.

Also provided herein are methods of enhancing innate immunity in a subject in need thereof, including administering to the subject a therapeutically effective amount of chemical entities described herein (e.g., compounds Formulae disclosed herein), or pharmaceutically acceptable salts, hydrates, solvates, or prodrugs thereof. In some embodiments, the compound is selected from the group consisting of: UDPS-Heptose, CDPS-Heptose, and ADPS-Heptose.

In some embodiments, the vaccine is a composition including, but not limited to an antigen of infectious agents, such as infectious bacterial, viral or parasitic pathogens, including Gram-negative bacterial pathogens belonging to the genus Neisseria (including Neisseria meningitidis, Neisseria gonorrohoeae), Escherichia (including Escherichia coli), Klebsiella (including Klebsiella pneumoniae), Salmonella (including Salmonella typhimurium), Shigella (including Shigella dysenteriae, Shigella flexneri, Shigella sonnei), Vibrio (including Vibrio cholerae), Helicobacter (including Helicobacter pylori), Pseudomonas (including Pseudo onas aeruginosa), Burkholderia (including Burkholderia multivorans), Haemophilus (including Haemophilus influenzae), Moraxella (including Moraxella catarrhalis), Bordetella (including Bordetella pertussis), Francisella (including Francisella tularensis), Pasteurella (including Pasteurella multocida), Legionella (including Legionella pneumophila), Borrelia (including Borrelia burgdorferi), Campylobacter (including Campylobacter jejuni), Yersinia (including Yersinia pestis and Yersinia enterocolitica), Rickettsia (including Rickettsia rickettsii), Treponema (including Treponema pallidum), Chlamydia (including Chlamydia trachomatis, Chlamydia pneumoniae) and Brucella spp., and including Gram positive bacterial pathogens belonging to the genus Staphylococcus (including Staphylococcus aureus), Streptococcus (including Streptococcus pneumoniae, Streptococcus pyogenes), Listeria (including Listeria monocytogenes), Corynebacterium (including Corynebacterium diphtheriae), Enterococcus (including Enterococcus faecalis), Clostridium spp., and Mycobacterium (including Mycobacterium tuberculosis, Mycobacterium leprae, Mycobacterium avium).

In some embodiments, the vaccine is a composition including, but not limited to an antigen of infectious agents, such as pathogenic viruses including Adenoviridae (including Adenovirus), Herpesviridae (including Epstein-Barr virus, Herpes Simplex Viruses, Cytomegalovirus, Varicella Zoster virus), Papillomviridae, Poxvi idae (including Papillomavirus), Hepadnaviridae (including Hepatitis B virus), Parvoviridae, Astroviridae, Caliciviridae, Picornaviridae (including Coxsackievirus, Hepatitis A virus, Poliovirus), Coronaviridae, Flaviviridae (including Hepatitis C virus, Dengue virus), Togaviridae (including Rubella virus), Hepeviridae, Retroviridae (including HIV), Orthomyxoviridae (including influenza virus, Arenaviridae, Bunyaviridae, Filoviridae, Paramyxoviridae (including Measles virus, Mumps virus, Parainfluenza virus, Respiratory Syncytial virus), Rhabdoviridae (including Rabies virus) or Reoviridae.

In some embodiments, a compound of Formulae disclosed herein, acts as a vaccine adjuvant for a vaccine in the treatment or prevention of anthrax, caries, pneumococcal disease, polio, rabies, rubella, Chagas disease, severe acute respiratory syndrome (SARS), shingles, smallpox, syphilis dengue, diphtheria, ehrlichiosis, hepatits A or B, herpes, seasonal influenza, Japanese encephalitis, leprosy, lyme disease, malaria, measles, mumps, meningococcal disease, including meningitis and septicemia, Onchocerciasis river blindness, pertussis (whooping cough), schistosomiasis, tetanus, tuberculosis, tularemia, tick-bome encephalitis virus, typhoid fever, trypanosomiasis, yellow fever, or visceral leishmaniasis.

In accordance with any of these embodiments, a compound of Formulae disclosed herein, and prodrugs, analogs and derivatives thereof, can serve as an adjuvant to a vaccine composition for the treatment or prevention of a disease or disorder caused by an infectious agent, or for the treatment of cancer as described herein, or for the treatment of another disease or disorder that may be treated with a vaccine composition, including, for example, Alzheimer's disease. In embodiments, the antigen is selected from amyloid protein in the treatment of Alzheimer's disease. In embodiments, the antigen is selected from glycoprotein 100 (gp100), mucin 1 (MUC1), and melanoma-associated antigen 3 (MAGEA3) in the treatment of cancer. In embodiments, the cancer is selected from breast, ovarian, hepatocellular cancer, or prostate cancer. In embodiments, the cancer is HTLV-1 T-lymphotropic leukemia.

In some embodiments, the vaccine is a composition including, but not limited to an antigen of infectious agents, such as pathogenic fungal infections including those caused by Candida, Aspergillus, Cryptococcus, Histoplasma, Pneumocystis, or Coccidioides.

In some embodiments for the treatment or prevention of an infectious disease, the compound of Formulae described herein, and prodrugs, analogs and derivatives thereof, serve as an adjuvant to a vaccine composition for the treatment or prevention of a disease or disorder caused by adenovirus, Coxsackie B virus, Haemophilus influenzae type b (Hib), hepatictis C virus (HCV), herpes virus, cytomegalovirus, eastern equine encephalitis virus, hookworm, Marburg virus, norovirus, respiratory syncytial virus (RSV), rotavirus, Ebola virus, enterovirus 71, Epstein-Barr virus, human immunodeficiency virus (HIV), human papillomavirus (HPV), Salmonella typhi, Staphylococcus aureus, Streptococcus pyogenes, varicella, West Nile virus, Yersinia pestis, and Zika virus.

### Combination Therapy

This disclosure contemplates both monotherapy regimens as well as combination therapy regimens. In some embodiments, the methods described herein can further include administering one or more additional therapies (e.g., one or more additional therapeutic agents or regimens (e.g., one or more immunotherapeutic agents and/or one or more immunotherapeutic regimens)) in combination with administration of the compounds described herein. The one or more additional therapeutic agents and/or regimens (e.g., immunotherapeutic agents and/or one or more immunotherapeutic regimens) can include examples generically or specifically described anywhere herein.

In certain embodiments, the methods described herein can further include administering one or more additional cancer therapies.

The one or more additional cancer therapies can include, without limitation, surgery, radiotherapy, chemotherapy, toxin therapy, immunotherapy, cryotherapy, cancer vaccines (e.g., HPV vaccine, hepatitis B vaccine, Oncophage, Provenge) and gene therapy, as well as combinations thereof. Immunotherapy, including, without limitation, adoptive cell therapy, the derivation of stem cells and/or dendritic cells, blood transfusions, lavages, and/or other treatments, including, without limitation, freezing a tumor.

In some embodiments, the one or more additional cancer therapies is chemotherapy, which can include administering one or more additional chemotherapeutic agents. In some embodiments, the one or more additional cancer therapies is immunotherapy, which can include administering one or more additional immunotherapeutic agents.

In certain embodiments, the additional immunotherapeutic agent is an immunomodulatory moiety, e.g., an immune checkpoint inhibitor. In certain of these embodiments, the immune checkpoint inhibitor targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine-TIM3, lymphocyte activation gene 3 protein (LAG3), MHC class II-LAG3, 4-1BB-4-1BB ligand, OX40-OX40 ligand, GITR, GITR ligand -GITR, CD27, CD70-CD27, TNFRSF25, TNFRSF25-TL1A, CD40L, CD40-CD40 ligand, HVEM-LIGHT-LTA, HVEM, HVEM-BTLA, HVEM-CD160, HVEM-LIGHT, HVEM-BTLA-CD160, CD80, CD80-PDL-1, PDL2-CD80, CD244, CD48-CD244, CD244, ICOS, ICOS—ICOS ligand, B7-H3, B7-H4, VISTA, TMIGD2, HHLA2, TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine-TIM3, SIRPA-CD47, VEGF, Neuropilin, CD160, CD30, and CD155; e.g., CTLA-4 or PD1 or PD-L1). See, e.g., Postow, M. J. Clin. Oncol. 2015, 33, 1.

In certain of these embodiments, the immune checkpoint inhibitor is selected from the group consisting of: Urelumab, PF-05082566, MEDI6469, TRX518, Varlilumab, CP-870893, Pembrolizumab (PD1), Nivolumab (PD1), Atezolizumab (formerly MPDL3280A) (PDL1), MEDI4736 (PD-L1), Avelumab (PD-L1), PDR001 (PD1), BMS-986016, MGA271, Lirilumab, IPH2201, Emactuzumab, INCB024360, Galunisertib, Ulocuplumab, BKT140, Bavituximab, CC-90002, Bevacizumab, and MNRP1685A, and MGA271.

In certain embodiments, the additional chemotherapeutic agent is an alkylating agent. Alkylating agents are so named because of their ability to alkylate many nucleophilic functional groups under conditions present in cells, including, but not limited to cancer cells. In a further embodiment, an alkylating agent includes, but is not limited to, Cisplatin, carboplatin, mechlorethamine, cyclophosphamide, chlorambucil, ifosfamide and/or oxaliplatin. In an embodiment, alkylating agents can function by impairing cell function by forming covalent bonds with the amino, carboxyl, sulfhydryl, and phosphate groups in biologically important molecules or they can work by modifying a cell's DNA. In a further embodiment an alkylating agent is a synthetic, semisynthetic or derivative.

In certain embodiments, the additional chemotherapeutic agent is an anti-metabolite. Anti-metabolites masquerade as purines or pyrimidines, the building-blocks of DNA and in general, prevent these substances from becoming incorporated in to DNA during the “S” phase (of the cell cycle), stopping normal development and division. Anti-metabolites can also affect RNA synthesis. In an embodiment, an antimetabolite includes, but is not limited to azathioprine and/or mercaptopurine. In a further embodiment an anti-metabolite is a synthetic, semisynthetic or derivative.

In certain embodiments, the additional chemotherapeutic agent is a plant alkaloid and/or terpenoid. These alkaloids are derived from plants and block cell division by, in general, preventing microtubule function. In an embodiment, a plant alkaloid and/or terpenoid is a vinca alkaloid, a podophyllotoxin and/or a taxane. Vinca alkaloids, in general, bind to specific sites on tubulin, inhibiting the assembly of tubulin into microtubules, generally during the M phase of the cell cycle. In an embodiment, a vinca alkaloid is derived, without limitation, from the Madagascar periwinkle, Catharanthus roseus (formerly known as Vinca rosea). In an embodiment, a vinca alkaloid includes, without limitation, Vincristine, Vinblastine, Vinorelbine and/or Vindesine. In an embodiment, a taxane includes, but is not limited, to Taxol, Paclitaxel and/or Docetaxel. In a further embodiment a plant alkaloid or terpernoid is a synthetic, semisynthetic or derivative. In a further embodiment, a podophyllotoxin is, without limitation, an etoposide and/or teniposide. In an embodiment, a taxane is, without limitation, docetaxel and/or ortataxel. In an embodiment, a cancer therapeutic is a topoisomerase. Topoisomerases are essential enzymes that maintain the topology of DNA. Inhibition of type I or type II topoisomerases interferes with both transcription and replication of DNA by upsetting proper

DNA supercoiling. In a further embodiment, a topoisomerase is, without limitation, a type I topoisomerase inhibitor or a type II topoisomerase inhibitor. In an embodiment a type I topoisomerase inhibitor is, without limitation, a camptothecin. In another embodiment, a camptothecin is, without limitation, exatecan, irinotecan, lurtotecan, topotecan, BNP 1350, CKD 602, DB 67 (AR67) and/or ST 1481. In an embodiment, a type II topoisomerase inhibitor is, without limitation, epipodophyllotoxin. In a further embodiment an epipodophyllotoxin is, without limitation, an amsacrine, etoposid, etoposide phosphate and/or teniposide. In a further embodiment a topoisomerase is a synthetic, semisynthetic or derivative, including those found in nature such as, without limitation, epipodophyllotoxins, substances naturally occurring in the root of American Mayapple (Podophyllum peltatum).

In certain embodiments, the additional chemotherapeutic agent is a stilbenoid. In a further embodiment, a stilbenoid includes, but is not limited to, Resveratrol, Piceatannol, Pinosylvin, Pterostilbene, Alpha-Viniferin, Ampelopsin A, Ampelopsin E, Diptoindonesin C, Diptoindonesin F, Epsilon-Vinferin, Flexuosol A, Gnetin H, Hemsleyanol D, Hopeaphenol, Trans-Diptoindonesin B, Astringin, Piceid and Diptoindonesin A. In a further embodiment a stilbenoid is a synthetic, semisynthetic or derivative.

In certain embodiments, the additional chemotherapeutic agent is a cytotoxic antibiotic. In an embodiment, a cytotoxic antibiotic is, without limitation, an actinomycin, an anthracenedione, an anthracycline, thalidomide, dichloroacetic acid, nicotinic acid, 2-deoxyglucose and/or chlofazimine. In an embodiment, an actinomycin is, without limitation, actinomycin D, bacitracin, colistin (polymyxin E) and/or polymyxin B. In another embodiment, an antracenedione is, without limitation, mitoxantrone and/or pixantrone. In a further embodiment, an anthracycline is, without limitation, bleomycin, doxorubicin (Adriamycin), daunorubicin (daunomycin), epirubicin, idarubicin, mitomycin, plicamycin and/or valrubicin. In a further embodiment a cytotoxic antibiotic is a synthetic, semisynthetic or derivative.

In certain embodiments, the additional chemotherapeutic agent is selected from abiraterone acetate, altretamine, anhydrovinblastine, auristatin, bexarotene, bicalutamide, BMS 184476, 2,3,4,5,6-pentafluoro-N-(3-fluoro-4-methoxyphenyl)benzene sulfonamide, bleomycin, N,N-dimethyl-L-valyl-L-valyl-N-methyl-L-valyl-L-proly-1-Lproline-t-butylamide, cachectin, cemadotin, chlorambucil, cyclophosphamide, 3′,4′-didehydro-4′-deoxy-8′-norvin-caleukoblastine, docetaxol, doxetaxel, cyclophosphamide, carboplatin, carmustine, cisplatin, cryptophycin, cyclophosphamide, cytarabine, dacarbazine (DTIC), dactinomycin, daunorubicin, decitabine dolastatin, doxorubicin (adriamycin), etoposide, 5-fluorouracil, finasteride, flutamide, hydroxyurea and hydroxyureataxanes, ifosfamide, liarozole, lonidamine, lomustine (CCNU), MDV3100, mechlorethamine (nitrogen mustard), melphalan, mivobulin isethionate, rhizoxin, sertenef, streptozocin, mitomycin, methotrexate, taxanes, nilutamide, onapristone, paclitaxel, prednimustine, procarbazine, RPR109881, stramustine phosphate, tamoxifen, tasonermin, taxol, tretinoin, vinblastine, vincristine, vindesine sulfate, and vinflunine.

In certain embodiments, the additional chemotherapeutic agent is platinum, cisplatin, carboplatin, oxaliplatin, mechlorethamine, cyclophosphamide, chlorambucil, azathioprine, mercaptopurine, vincristine, vinblastine, vinorelbine, vindesine, etoposide and teniposide, paclitaxel, docetaxel, irinotecan, topotecan, amsacrine, etoposide, etoposide phosphate, teniposide, 5-fluorouracil, leucovorin, methotrexate, gemcitabine, taxane, leucovorin, mitomycin C, tegafur-uracil, idarubicin, fludarabine, mitoxantrone, ifosfamide and doxorubicin. Additional agents include inhibitors of mTOR (mammalian target of rapamycin), including but not limited to rapamycin, everolimus, temsirolimus and deforolimus.

In still other embodiments, the additional chemotherapeutic agent can be selected from those delineated in U.S. Pat. No. 7,927,613, which is incorporated herein by reference in its entirety.

In certain embodiments, the additional therapeutic agent is a chemotherapeutic and/or immunotherapeutic agent which is selected from the group consisting of endostatin, angiogenin, angiostatin, chemokines, angioarrestin, angiostatin (plasminogen fragment), basement-membrane collagen-derived anti-angiogenic factors (tumstatin, canstatin, or arrestin), anti-angiogenic antithrombin III, signal transduction inhibitors, cartilage-derived inhibitor (CDI), CD59 complement fragment, fibronectin fragment, gro-beta, heparinases, heparin hexasaccharide fragment, human chorionic gonadotropin (hCG), interferon alpha/beta/gamma, interferon inducible protein (IP-10), interleukin-12, kringle 5 (plasminogen fragment), metalloproteinase inhibitors (TIMPs), 2-methoxyestradiol, placental ribonuclease inhibitor, plasminogen activator inhibitor, platelet factor-4 (PF4), prolactin 16 kD fragment, proliferin-related protein (PRP), various retinoids, tetrahydrocortisol-S, thrombospondin-1 (TSP-1), transforming growth factor-beta (TGF-β), vasculostatin, vasostatin (calreticulin fragment) and the like.

In certain embodiments, the additional therapeutic agent is an anti-cancer antibody. Non-limiting examples include those described generically or specifically in the table infra.

| Human Antigen | Antibody ( commercial or scientific name) |
|---|---|
| CD2 | Siplizumab |
| CD3 | UCHT1 |
| CD4 | HuMax-CD4 |
| CD19 | SAR3419, MEDI-551 |
| CD19 and CD3 or CD22 | Bispecific antibodies such as Blinatumomab, DT2219ARL |
| CD20 | Rituximab, Veltuzumab, Tositumomab, Ofatumumab, |
| | Ibritumomab, Obinutuzumab, |
| CD22 (SIGLEC2) | Inotuzumab, tetraxetan, CAT-8015, DCDT2980S, |
| | Bectumomab |
| CD30 | Brentuximab vedotin |
| CD33 | Gemtuzumab ozogamicin (Mylotarg) |
| CD37 | TRU-016 |
| CD38 | Daratumumab |
| CD40 | Lucatumumab |
| CD52 | Alemtuzumab (Campath) |
| CD56 (NCAM1) | Lorvotuzumab |
| CD66e (CEA) | Labetuzumab |
| CD70 | SGN-75 |
| CD74 | Milatuzumab |
| CD138 (SYND1) | BT062 |
| CD152 (CTLA-4) | Ipilimumab |
| CD221 (IGF1R) | AVE1642, IMC-A12, MK-0646, R150, CP 751871 |
| CD254 (RANKL) | Denosumab |
| CD26 1 (TRAILR1) | Mapatumumab |
| CD262 (TRAILR2) | HGS-ETR2, CS-1008 |
| CD326 (Epcam) | Edrecolomab, 17-1A, IGN101, Catumaxomab, |
| | Adecatumumab |
| CD309 (VEGFR2) | IM-2C6, CDP791 |
| CD319 (SLAMF7) | HuLuc63 |
| CD340 (HER2) | Trastuzumab, Pertuzumab, Ado-trastuzumab emtansine |
| CAIX (CA9) | cG250 |
| EGFR | Cetuximab, Panitumumab, nimotuzumab and 806 |
| EPHA3 (HEK) | KB004, IIIA4 |
| Episialin | Epitumomab |
| FAP | Sibrotuzumab and F19 |
| HLA-DR beta | Apolizumab |
| FOLR-1 | Farletuzumab |
| 5T4 | Anatumomab |
| GD3/GD2 | 3F8, ch14.18, KW-2871 |
| gpA33 | huA33 |
| GPNMB | Glembatumumab |
| HER3 | MM-121 |
| Integrin αVβ3 | Etaracizumab |
| Integrin α5β1 | Volociximab |
| Lewis-Y antigen | hu3S193, 1gN311 |
| MET(HGFR) | AMG 102, METMAB |
| Mucin-1/CanAg | Pemtumomab, oregovomab, Cantuzumab |
| PSMA | ADC J591 |
| Phosphatidylserine | Bavituximab |
| TAG-72 | Minretumomab |
| Tenascin | 81C6 |
| VEGF | Bevacizumab |
| PD-L1 | Avelumab |
| CD274 | Durvalumab |

In certain embodiments, the additional therapeutic agent or regimen is administered to the subject prior to contacting with or administering the chemical entity (e.g., about one hour prior, or about 6 hours prior, or about 12 hours prior, or about 24 hours prior, or about 48 hours prior, or about 1 week prior, or about 1 month prior).

In other embodiments, the additional therapeutic agent or regimen is administered to the subject at about the same time as contacting with or administering the chemical entity. By way of example, the additional therapeutic agent or regimen and the chemical entity are provided to the subject simultaneously in the same dosage form. As another example, the additional therapeutic agent or regimen and the chemical entity are provided to the subject concurrently in separate dosage forms.

In still other embodiments, the additional therapeutic agent or regimen is administered to the subject after contacting with or administering the chemical entity (e.g., about one hour after, or about 6 hours after, or about 12 hours after, or about 24 hours after, or about 48 hours after, or about 1 week after, or about 1 month after).

### Patient Selection

In some embodiments, the methods described herein further include the step of identifying a subject (e.g., a patient) in need of such treatment (e.g., by way of biopsy, endoscopy, or other conventional method known in the art). In certain embodiments, the ALPK1 protein can serve as a biomarker for certain types of cancer, e.g., hepatocellular cancer, colon cancer and prostate cancer. In other embodiments, identifying a subject can include assaying the patient's tumor microenvironment for the absence of T-cells and/or presence of exhausted T-cells, e.g., patients having one or more cold tumors. Such patients can include those that are resistant to treatment with checkpoint inhibitors. In certain embodiments, such patients can be treated with a chemical entity herein, e.g., to recruit T-cells into the tumor, and in some cases, further treated with one or more checkpoint inhibitors, e.g., once the T-cells become exhausted.

In some embodiments, the chemical entities, methods, and compositions described herein can be administered to certain treatment-resistant patient populations (e.g., patients resistant to checkpoint inhibitors; e.g., patients having one or more cold tumors, e.g., tumors lacking T-cells or exhausted T-cells).

### Compound Preparation

As can be appreciated by the skilled artisan, methods of synthesizing the compounds of the formulae described herein will be evident to those of ordinary skill in the art. For example, the compounds described herein can be synthesized, e.g., using one or more of the methods described herein. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and RGM. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof. The starting materials used in preparing the compounds described herein are known, made by known methods, or are commercially available. The skilled artisan will also recognize that conditions and reagents described herein that can be interchanged with alternative art-recognized equivalents. For example, in many reactions, triethylamine can be interchanged with other bases, such as non-nucleophilic bases (e.g. diisopropylethylamine, 1,8-diazabicycloundec-7-ene, 2,6-di-tert-butylpyridine, or tetrabutylphosphazene).

The skilled artisan will recognize a variety of analytical methods that can be used to characterize the compounds described herein, including, for example, ¹H NMR, heteronuclear NMR, mass spectrometry, liquid chromatography, and infrared spectroscopy. The foregoing list is a subset of characterization methods available to a skilled artisan and is not intended to be limiting.

### General Synthetic Schemes

For example, the compounds of Formula (X), and sub-Formulae can be synthesized as illustrated in Schemes 1 to 11.

### Preparation of Compounds of Formula I and Exemplary Compounds

The compounds of formula I (compound I) can be made by general synthetic method as illustrated in Scheme 1. Compound I-b (R refers to a protection group) can be obtained by reacting compound I-a with protected phosphorochloridate under basic condition or appropriate protected phosphate under Mitsunobu reaction condition. Compound I-b can be obtained as a mixture of alpha and beta isomers which can be separated on silica gel chromatography. The beta isomer of compound I-b is deprotected under 1-4 atm of H2 catalyzed by Pd/C or PtO₂ to give compound I-c. Coupling of compound I-c with 1,1′-carbonyldiimidazole (CDI) in an appropriate solution such as N,N-dimethylformamide (DMF) to give compound I-d. Coupling of compound I-e and compound I-d in an appropriate solvent such as DMF with an appropriate catalyst such as zinc (II) chloride under room temperature provides compound I. The compound I where R² has a different stereochemistry can be obtained starting from the stereoisomer of compound I-a.

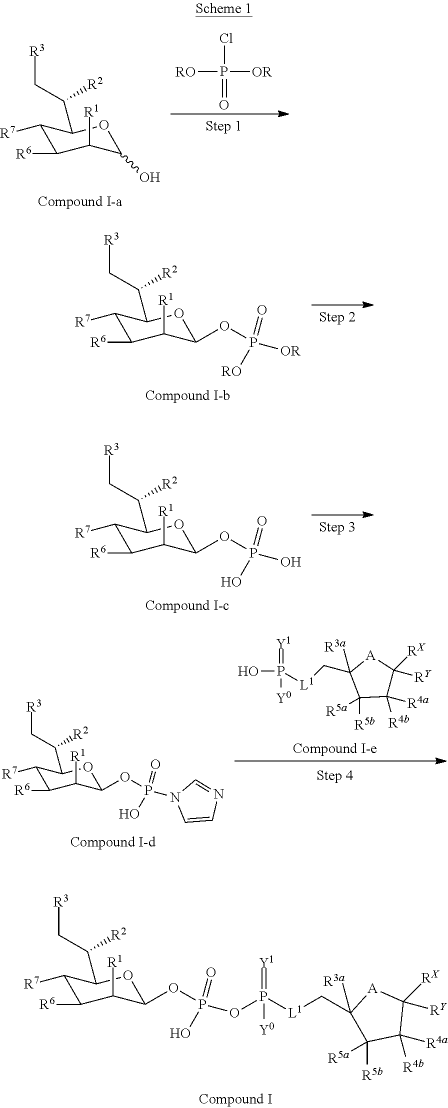

The compounds of formula I (compound II) can be made by general synthetic method as illustrated in Scheme 2. The unprotected hydroxy group in compound 1 can be oxidated in the presence of oxidation reagent to give compound 2, which is treated with Grignard reagent to form compound 3. The resulting hydroxy group can be protected with BzCl to form compound 4, which is coupled with HR^{X} wherein R^{X} is for example a base group to form compound 5. And the protection group in compound 5 can be removed in the basic condition to get compound 6, which is treated with PSCl₃ to form phosphorothioatecompound 7. And finally coupling of compound 7 and compound 8 in an appropriate solvent such as DMF with an appropriate catalyst such as zinc (II) chloride under room temperature provides compound II.

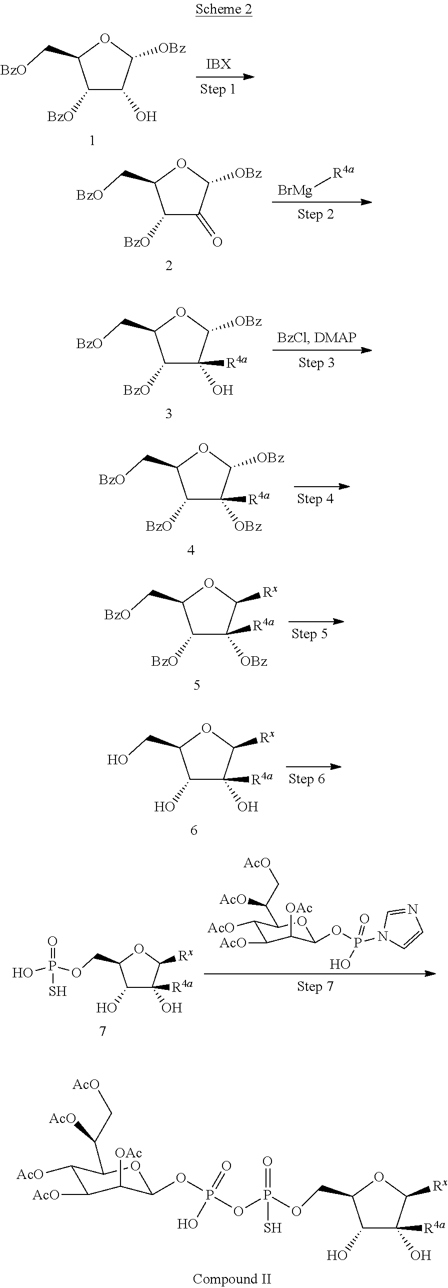

The compounds of formula I (compound III) can be made by general synthetic method as illustrated in Scheme 3. Two hydroxy groups in compound 1 can be protected to form compound 2, which was treated with Tf₂O in the presence of DMAP to produce compound 3. The OTf group in compound 3 was treated with sodium azide to form compound 4, and then the protection group was removed in the presence of fluorine reagent to get compound 5. Phosphorothioatecompound 6 can be formed from compound 5 and PSCl₃ in low temperature, which was coupled with compound 8 to form compound 7. And the azide group was reduced to amino to form final product compound III.

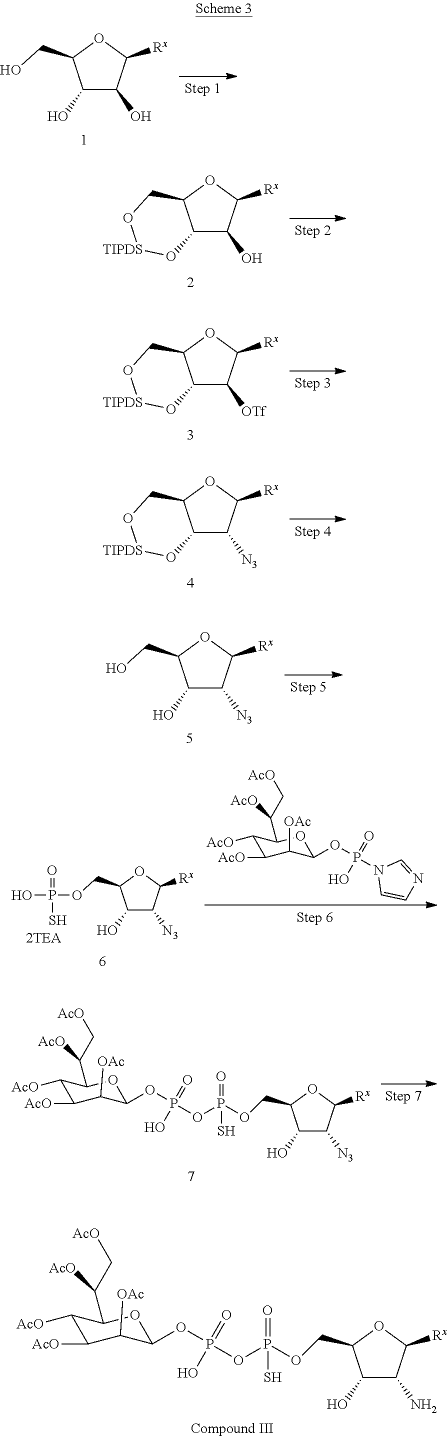

The compounds of formula I (compound IV) can be made by general synthetic method as illustrated in Scheme 4. The commercially available compound 1 was treated with PSCl₃ in low temperature to form compound 2, which coupled with compound 8 in the presence catalyst to form final compound IV.

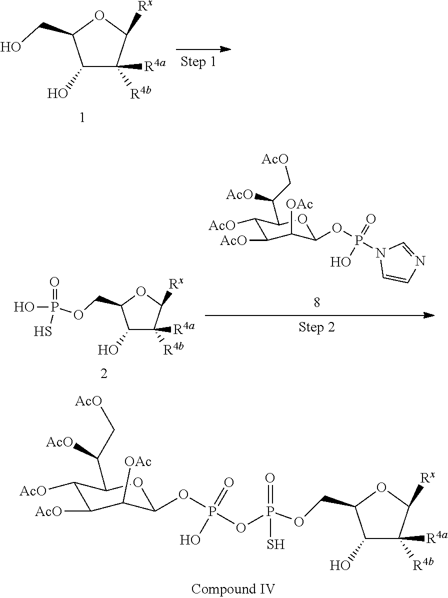

The preparation of compounds with 2′-F di-substitutions (compound V), 2′-OMe di-substitutions (compound VI), 3′-substitutions (compound VII), 4′-F and 2′-di-substitutions (compound VIII), 4′-Me and 2′-di-substitutions (compound IX), 2′-disubstitutions (compound X), and 2′-disubstitutions and 3′-substitutions (compound XI) are shown in the Schemes 5 to 11 below.

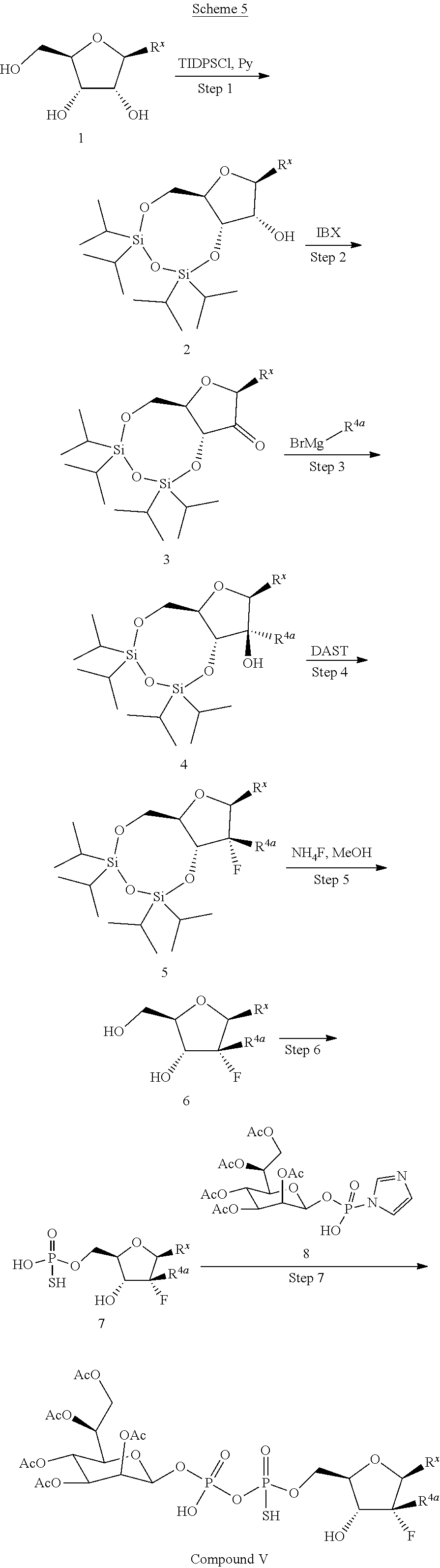

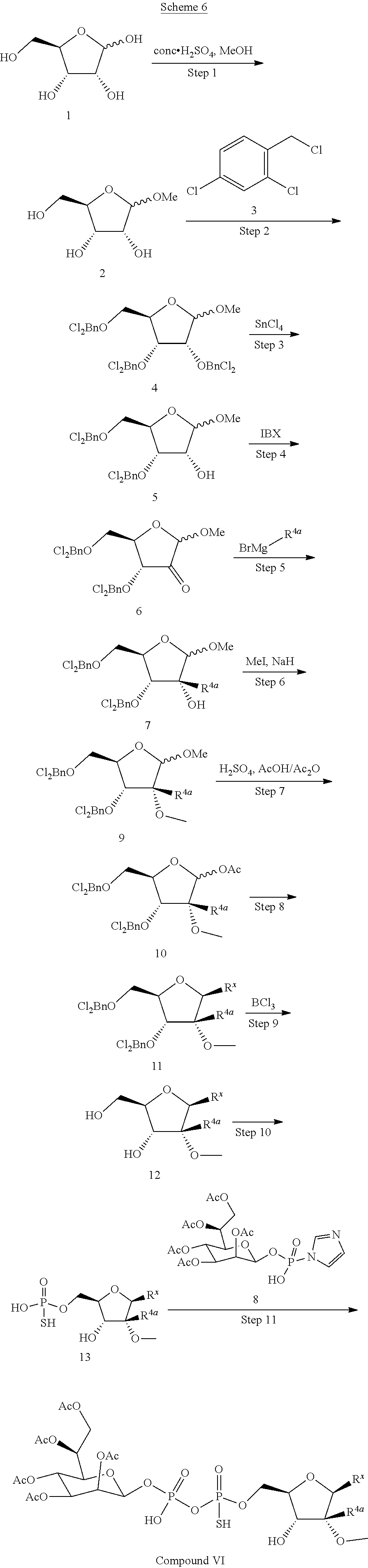

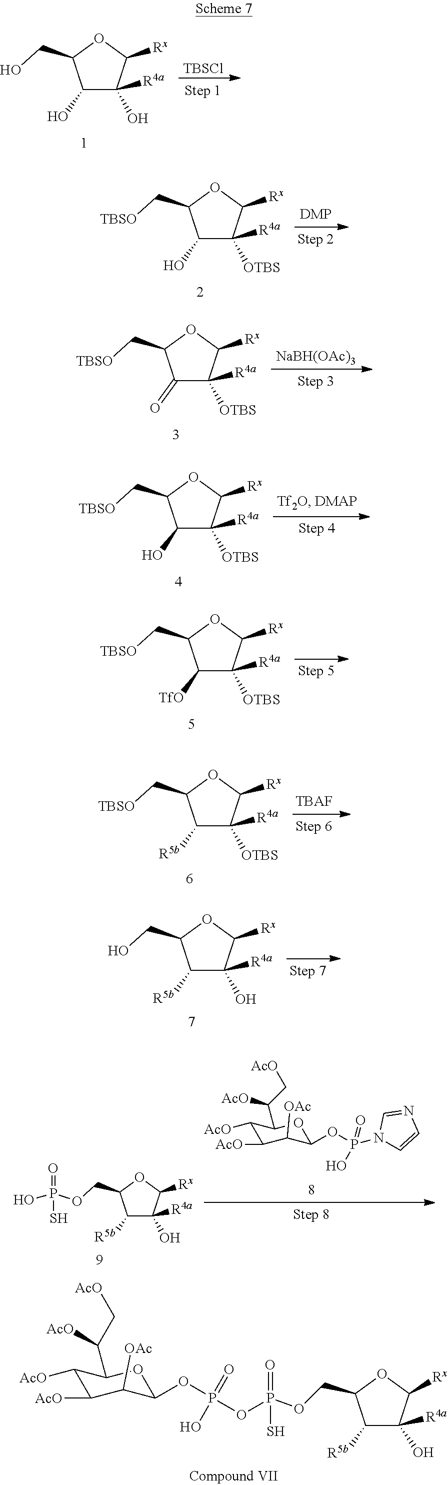

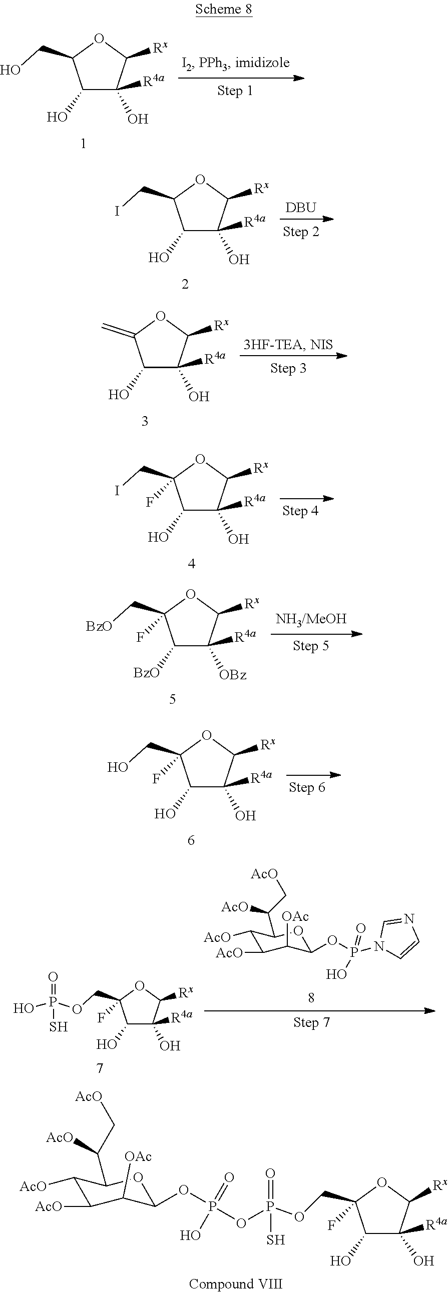

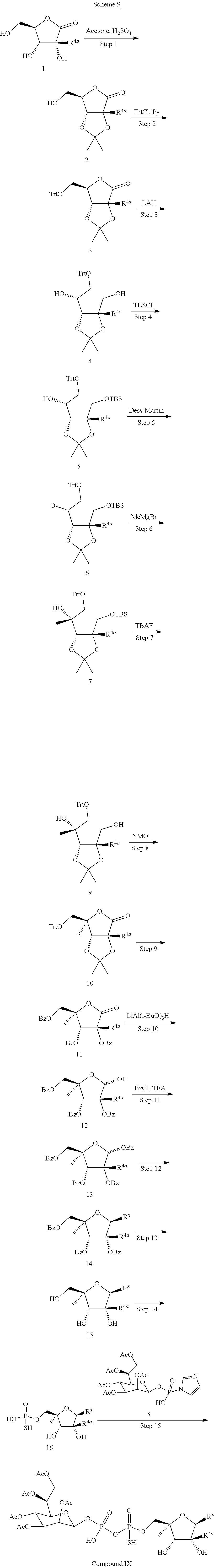

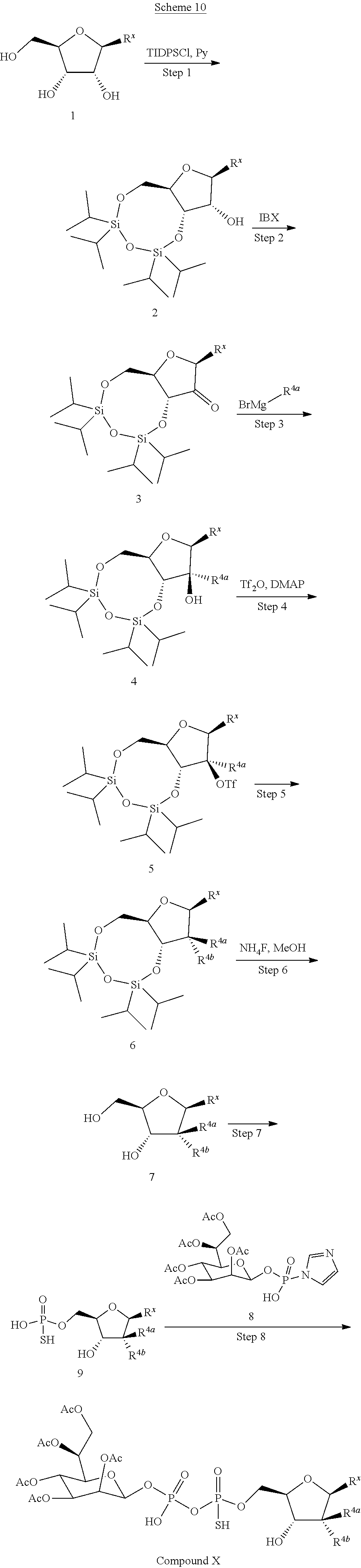

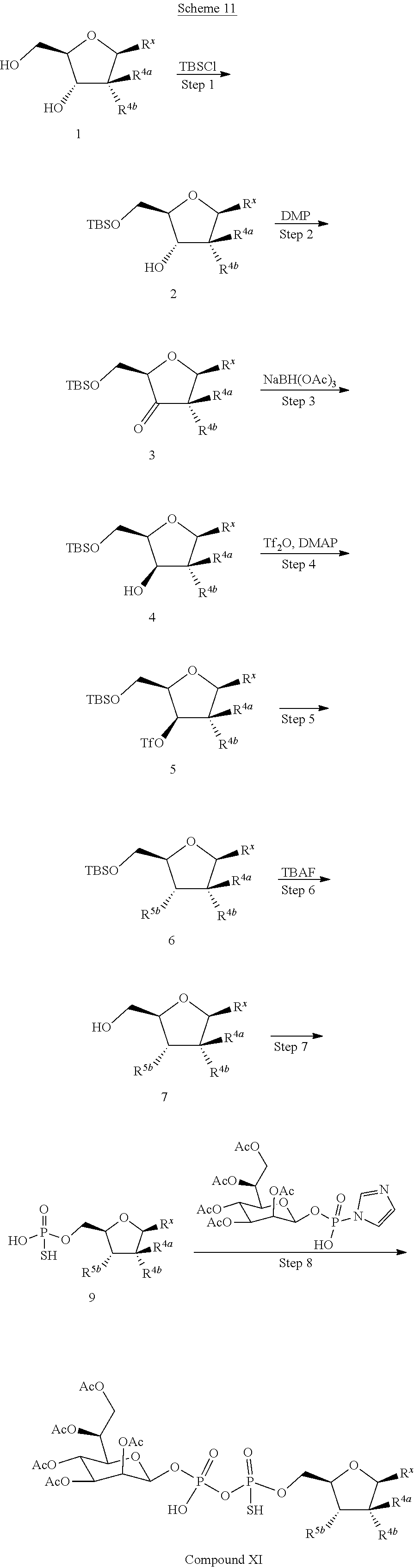

### Preparative Examples

¹H NMR spectra were recorded on a Varian instrument operating at 400 MHz. ¹HNMR spectra were obtained using CDCl₃, CD₂Cl₂, CD₃OD, D20, de-DMSO, de-acetone or (CD₃)₂CO as solvent and tetramethylsilane (0.00 ppm) or residual solvent (CDCl₃:7.25 ppm; CD₃OD: 3.31 ppm; D20:4.79 ppm; d6-DMSO: 2.50 ppm; de-acetone or (CD₃)₂CO: 2.05) as the reference standard. When peak multiplicities are reported, the following abbreviations are used: s (singlet), d (doublet), t (triplet), q (quartet), qn (quintuplet), sx (sextuplet), m (multiplet), br (broadened), dd (doublet of doublets), dt (doublet of triplets). Coupling constants, when given, are reported in Hertz (Hz). All compound names except the reagents were generated by Chemdraw version 12.0.

In the following examples, the abbreviations below are used:
AcOH Acetic acid
aq Aqueous
Brine Saturated aqueous sodium chloride solution
CH₂Cl₂ Dichloromethane
DMF N,N-Dimethylformamide
Dppf 1,1′-bis(diphenylphosphino) ferrocene
DBU 1,8-diazabicyclo[5.4.0]undec-7-ene
DIEA N,N-diisopropylethylamine
DMAP 4-(N,N-dimethylamino) pyridine
DMF N,N-dimethylformamide
DMSO Dimethyl sulfoxide
eq Equivalent
EtOAc Ethyl acetate
EtOH Ethanol

Et₂O or ether Diethyl ether
g grams
h or hr hour
HATU 2-(1H-7-Azabenzotriazol-1-yl)-1,1,3,3-tetramethyl uronium hexafluorophosphate Methanaminium
HCl Hydrochloric acid
HPLC High-performance liquid chromatography
IPA or i-PrOH 2-propanol
mg milligrams
mL or ml milliliters
mmol millimole
MeCN Acetonitrile
MeOH Methanol
Min minutes
ms or MS Mass spectrum
Na₂SO₄ Sodium sulfate
PPA Polyphosphoric acid
PPh₃ Triphenylphosphine
PSCl₃ Thiophosphoryl trichloride
Rt Retention time
rt Room temperature
TEAB Triethylamonium bicarbonate
TFA Trifluoroacetic acid
THF tetrahydrofuran
TLC thin layer chromatography
TMSCl Trimethylsilyl chloride
μL or μl Microliters

### Example 1C

### Synthesis of Example 1C

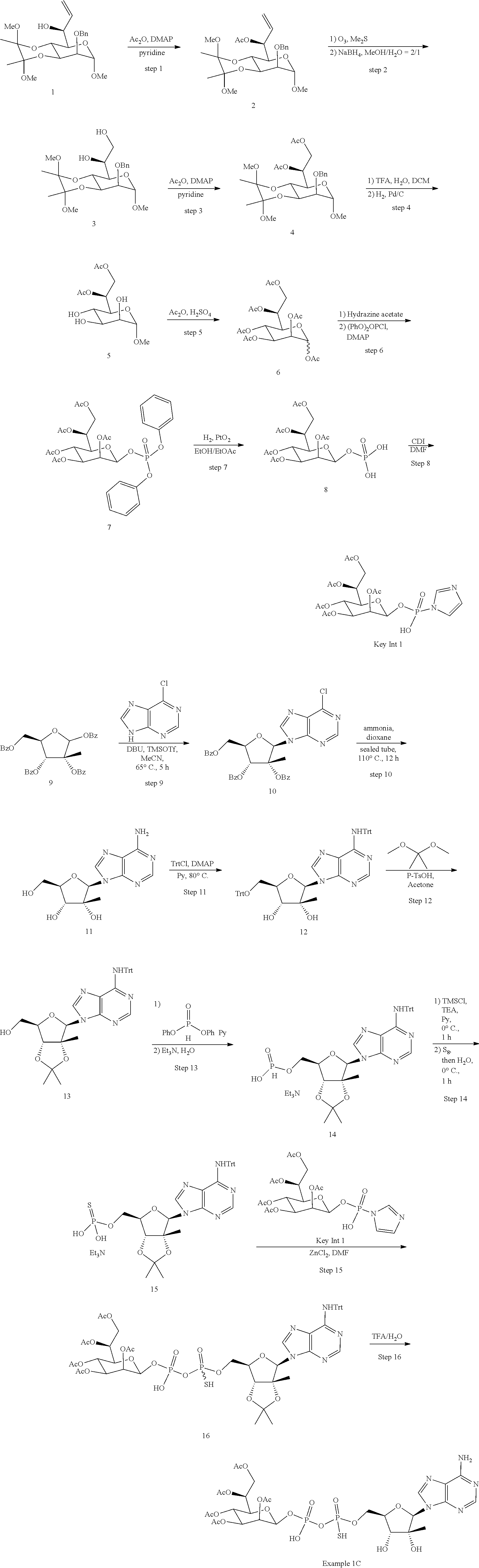

### Step 1: Synthesis of Compound 2

To a solution of compound 1 (13 g, 30.63 mmol) in pyridine (65 mL) was added DMAP (374 mg, 3.06 mmol), followed by Ac₂O (6.25 g, 61.25 mmol), the mixture was stirred at rt for 2 h, the desired product was found based on LCMS. EA (100 mL) was added to the reaction, which was washed with 1N HCl (100 mL×2), dried, concentrated and purified with column chromatography (PE/EA=3/1) to give the title compound (14 g, 93%) as a white solid. ¹H NMR (400 MHz, DMSO-d₆) δ 7.41 (d, J=7.0 Hz, 2H), 7.38-7.33 (m, 2H), 7.31-7.26 (m, 1H), 5.96-5.86 (m, 1H), 5.44-5.39 (m, 1H), 5.27-5.17 (m, 2H), 4.79-4.71 (m, 2H), 4.64 (d, J=12.2 Hz, 1H), 4.03-3.96 (m, 1H), 3.87 (dd, J=10.2, 3.0 Hz, 1H), 3.67-3.61 (m, 2H), 3.23 (s, 3H), 3.15 (s, 3H), 3.06 (s, 3H), 2.06 (s, 3H), 1.23 (s, 3H), 1.16 (s, 3H).

### Step 2: Synthesis of Compound 3

A mixture of compound 2 (14 g, 30 mmol) in DCM/MeOH=1:1 (280 mL) was stirred at −78° C. for 40 min under O₃ atmosphere, then quenched by (CH₃) 2S (11.2 mL), and the mixture was stirred at rt overnight. The mixture was concentrated and dissolved in MeOH: H₂O-2:1 (240 mL), NaBH₄ (4.5 g, 120 mmol) was added at 0° C., then warmed to rt, stirred for 2 h, the desired product was found based on LCMS, the reaction was concentrated and extracted with DCM (100 mL×2), the combined organic layer was dried and concentrated to give the title compound (13 g, 90%) as a colorless oil. MS (ESI) m/z [M+H]⁺450.9.

### Step 3: Synthesis of Compound 4

To a solution of compound 3 (13.6 g, 31.74 mmol) in Pyridine (100 mL) was added DMAP (388 mg, 3.17 mmol), followed by Ac₂O (9.7 g, 95.22 mmol). The mixture was stirred at rt for 3 h, the desired product was found based on TLC(PE/EA=1:1), EA (100 mL) was added, washed with 1 N HCl (100 mL×2), and the organic layer was dried over Na₂SO₄, filtered, concentrated and the crude product (16 g) was used in the next step without further purification.

### Step 4: Synthesis of Compound 5

The crude product from step 3 (16 g) was dissolved in DCM (250 mL), to which TFA (50 mL) and water (5 mL) was added, stirred for 2h, concentrated and the residue was dissolved in EtOH (200 mL), Pd/C(1.6 g, 10% wt) was added and the mixture was stirred under H₂ atmosphere at rt overnight, filtered and concentrated, and the residue was purified with CombiFlash (40 g, EA in PE 0˜40%) to give the title compound (5.6 g, 54% for 2 steps) as a colorless oil. ¹H NMR (400 MHz, CDCl₃) δ 5.45-5.37 (m, 1H), 4.77-4.74 (m, 1H), 4.40-4.28 (m, 2H), 3.99-3.94 (m, 1H), 3.87-3.79 (m, 1H), 3.72-3.67 (m, 1H), 3.58-3.51 (m, 1H), 3.36 (s, 3H), 2.20 (s, 3H), 2.07 (s, 3H).

### Step 5: Synthesis of Compound 6

To a solution of compound 5 (5.6 g, 18.16 mmol) in Ac₂O (25 mL) was added con. H2SO₄ (0.25 mL) at 0° C., then the mixture was stirred at rt for 3h, monitored by TLC(PE/EA=2/3), water (50 mL) was added, extracted with EA (50 mL×2), the organic layers were combined and washed with brine, dried, concentrated and used in the next step without further purification. ¹H NMR (400 MHz, CDCl₃) δ 6.10 (d, J=1.7 Hz, 1H), 5.36-5.31 (m, 2H), 5.28-5.22 (m, 2H), 4.27 (dd, J=11.6, 5.1 Hz, 1H), 4.21-4.14 (m, 2H), 2.20 (s, 3H), 2.17 (s, 3H), 2.14 (s, 3H), 2.03 (d, J=1.6 Hz, 6H), 2.00 (s, 3H).

### Step 6: Synthesis of Compound 7

To a solution of compound 6 (8 g, 17.3 mmol) in DMF (50 mL) was added Hydrazine acetate (2.4 g, 25.95 mmol). The mixture was stirred at rt for 2 h. The reaction was quenched with water (100 mL), extracted with EA (50 mL×2). The combined organic layer was washed with brine, dried and concentrated. The crude product was dissolved in DCM (140 mL), DMAP (8.4 g, 68.51 mmol) in DCM (140 mL) was added, followed by addition of diphenyl chlorophosphonate (4.8 g, 17.98 mmol) in DCM (100 mL) over 6 h. Then the mixture was stirred at rt overnight, monitored by TLC, concentrated and purified with Flash (80 g, EA in PE 0˜30%) to give the title compound (900 mg, 8%) as a colorless oil. ¹H NMR (400 MHz, CDCl₃) δ 7.39-7.27 (m, 6H), 7.25-7.19 (m, 2H), 7.18-7.13 (m, 2H), 5.57 (dd, J=7.0, 1.1 Hz, 1H), 5.52 (d, J=3.2 Hz, 1H), 5.35-5.30 (m, 1H), 5.28-5.24 (m, 1H), 5.06 (dd, J=10.1, 3.3 Hz, 1H), 4.26 (dd, J=11.5, 5.2 Hz, 1H), 4.15-4.11 (m, 1H), 3.83 (dd, J=10.0, 2.4 Hz, 1H), 2.13 (d, J=0.7 Hz, 6H), 2.02 (d, J=2.0 Hz, 6H), 1.98 (s, 3H).

### Step 7: Synthesis of Compound 8

To a solution of compound 7 (900 mg, 1.38 mmol) in EtOH/EA=1/1 (18 mL) was added PtO₂ (156 mg), the mixture was stirred at rt overnight under H₂ atmosphere, filtered and concentrated to give the title compound (600 mg) as a white solid. ¹H NMR (400 MHz, CDCl₃) δ 5.58-5.55 (m, 1H), 5.52-5.48 (m, 1H), 5.35-5.32 (m, 1H), 5.30-5.26 (m, 1H), 5.20-5.15 (m, 1H), 4.48-4.42 (m, 1H), 4.23-4.17 (m, 1H), 3.91 (dd, J=9.9, 2.3 Hz, 1H), 2.24 (s, 3H), 2.13 (s, 3H), 2.08 (s, 3H), 2.03 (s, 3H), 1.99 (s, 3H).

### Step 8: Synthesis of Key Int 1

To a solution of compound 8 (600 mg, 0.93 mmol) in DMF (5 mL) charged with N₂ was added CDI (1.5 g, 9.32 mmol), the mixture was stirred at rt for 3 h, the desired product was found based on LCMS with complete conversion. The reaction was quenched with MeOH (1 mL) and stirred at rt for 10 min, concentrated and the crude product was used in the next step without further purification. MS (ESI) m/z [M+H]⁺550.9; [M−H]-549.1.

### Step 9: Synthesis of Compound 10

To a solution of compound 9 (5 g, 8.6 mmol) and 6-chloro-9H-purine (1.3 g, 8.6 mmol) in ACN (80 mL) was added N,O-Bis(trimethylsilyl) acetamide (5.6 g, 27.5 mmol) at 25° C. To this solution was added TMSOTf (8.23 g, 37 mmol) at 0° C. The mixture was stirred for 1 h at 25° C. and then 1 h at 60° C., the desired product was found based on LCMS. The reaction was quenched with aqueous sodium bicarbonate (20 mL), extracted with EA (30 mL×3). The combined organic layer was dried over Na₂SO₄, filtered, concentrated and purified with column chromatography (EA in PE 0˜50%) to give the title compound (3 g, 51.1%) as a yellow oil. MS (ESI) m/z [M+H]⁺612.6.

### Step 10: Synthesis of Compound 11

To a solution of compound 10 (1.4 g, 2.28 mmol) in dioxane (15 mL) was added NH₃H2O 2O (45 mL). The mixture was stirred at 110° C. in sealed tube overnight. The mixture was concentrated to give the crude, which was washed with DCM to give the title compound (1.2 g, 93%) as a yellow solid. ¹H NMR (400 MHz, DMSO-d₆) δ 8.44 (s, 1H), 8.10 (s, 1H), 7.30-7.23 (m, 2H), 5.91 (s, 1H), 4.04 (d, J=9.1 Hz, 1H), 3.91-3.85 (m, 1H), 3.82-3.77 (m, 1H), 3.69-3.63 (m, 1H), 0.73 (s, 3H). MS (ESI) m/z [M+H]⁺282.0.

### Step 11: Synthesis of Compound 12

To a solution of compound 11 (630 mg, 2.23 mmol) in pyridine (10 mL) was added TrtCl (1.56 g, 5.6 mmol) and DMAP (219 mg, 1.79 mmol). The reaction was stirred at 80° C. for 16 h. Then it was concentrated and purified with column chromatography (EA in PE 0˜100%) to give the title compound (950 mg, 47%) as a colorless oil. ¹H NMR (400 MHz, DMSO-d₆) δ 8.33 (s, 1H), 7.80 (s, 1H), 7.50 (s, 1H), 7.43-7.17 (m, 30H), 5.99 (s, 1H), 5.33-5.17 (m, 2H), 4.28-4.16 (m, 1H), 4.14-4.05 (m, 1H), 0.84 (s, 3H). MS (ESI) m/z [M+H]⁺765.7.

### Step 12: Synthesis of Compound 13

To a solution of compound 12 (950 mg, 1.24 mmol) in acetone (45 mL) was added 2,2-dimethoxypropane (9 mL) and p-TsOH·H2O 2O (282.8 mg, 1.45 mmol). The reaction mixture was stirred at 25° C. overnight. Then it was diluted with brine and carefully quenched with saturated NaHCO₃ (30 mL), extracted with EtOAc (50 mL×3). The combined organic layer was died over MgSO₄, filtered and the filtrate was concentrated and purified with column chromatography (EA in PE 0˜80%) to give the title compound (550 mg, 70%) as a colorless oil. ¹H NMR (400 MHz, MeOD) δ 8.30 (s, 1H), 7.77 (s, 1H), 7.29-7.08 (m, 15H), 6.19 (s, 1H), 4.55 (d, J=2.2 Hz, 1H), 4.29-4.17 (m, 1H), 3.85-3.75 (m, 1H), 3.72-3.66 (m, 1H), 1.52 (s, 3H), 1.31 (s, 3H), 1.12-1.04 (m, 3H). MS (ESI) m/z [M+H]⁺563.8.

### Step 13: Synthesis of Compound 14

To a solution of compound 13 (320 mg, 0.56 mmol) in Pyridine (5 mL) was added diphenyl phosphonate (532 mg, 2.27 mmol). The reaction was stirred at 25° C. for 2 h. Then TEA (344 mg, 3.4 mmol) and H₂O 2O (122.7 mg, 2.8 mmol) was added and stirred at 25° C. for 0.5h. The resulting mixture was concentrated to give the crude. The residue was applied onto a silica gel column eluting with DCM/MeOH (10/1) to give the title compound (1.5 g, purity 20%, 84%) as a colorless oil. ¹H NMR (400 MHz, DMSO-d₆) δ 8.55 (s, 1H), 7.92 (s, 1H), 7.32-7.16 (m, 15H), 6.22 (s, 1H), 4.67-4.56 (m, 1H), 4.36-4.29 (m, 1H), 4.03-3.85 (m, 2H), 1.54 (s, 3H), 1.35 (s, 3H), 1.27-1.20 (m, 3H). MS (ESI) m/z [M+H]⁺627.8.

### Step 14: Synthesis of Compound 15

To a solution of compound 14 (600 mg, 0.95 mol) in Pyridine (6 mL) and TEA (6 mL) was added TMSCl (830.8 mg, 7.65 mmol). The reaction was stirred at 0° C. for 2 h. Then S8 (290.6 mg, 9.08 mmol) was added. The mixture was stirred at 0° C. for 1h. It was quenched with H2O, then concentrated and purified by prep-HPLC(water with 0.5% TFA in MeCN=75% to 40%) to give the title compound (300 mg, 45%) as a white solid. ¹H NMR (400 MHz, DMSO-d₆) δ 7.88 (s, 1H), 7.42 (s, 1H), 7.32-7.10 (m, 15H), 6.20 (s, 1H), 4.67 (s, 1H), 4.35 (s, 1H), 4.02-3.84 (m, 2H), 1.49 (s, 3H), 1.30 (s, 3H), 1.19-1.09 (m, 3H). MS (ESI) m/z [M+H]⁺659.6.

### Step 15: Synthesis of Compound 16

To a mixture of compound 15 (30 mg, 0.04 mmol) and Key Int 1 (28 mg, 0.05 mmol) in DMF (1.5 mL) was added ZnCl₂ (78.1 mg, 0.57 mmol). The mixture was stirred at 25° C. overnight. Then it was concentrated and purified with Prep-HPLC(10 mM aqueous solution of NH₄HCO₃ in MeCN=70% to 40%) to give the title compound (40 mg, 68%) as a white solid.

¹H NMR (400 MHz, DMSO-d₆) δ 8.70-8.55 (m, 1H), 7.90 (s, 1H), 7.47-7.02 (m, 15H), 6.22 (s, 1H), 5.75-5.55 (m, 1H), 5.48-5.29 (m, 1H), 5.22-4.95 (m, 3H), 4.88-4.62 (m, 1H), 4.44-4.27 (m, 2H), 4.23-3.90 (m, 4H), 2.14-1.80 (m, 12H), 1.52 (s, 3H), 1.36-1.00 (m, 6H). MS (ESI) m/z [M+H]⁺1141.7.

### Step 16: Synthesis of Example 1C

To a solution of compound 16 (40 mg, 0.035 mmol) in H2O 2O (2 mL) was added TFA (3 mL). The mixture was stirred at rt for 1 h. The reaction solution was adjusted to pH 7 with TEA, purified with prep-HPLC(10 mM NH₄HCO₃ aqueous solution in MeCN=90% to 70%) to give Example 1C(10 mg, 30%) as a white solid. ¹H NMR (400 MHz, MeOD) δ 8.81-8.64 (m, 1H), 8.17 (s, 1H), 6.15-6.05 (m, 1H), 5.71-5.49 (m, 2H), 5.33-5.20 (m, 1H), 5.20-5.06 (m, 2H), 4.61-4.49 (m, 1H), 4.48-4.35 (m, 2H), 4.33-4.21 (m, 2H), 4.20-4.11 (m, 1H), 4.02-3.78 (m, 1H), 2.24-2.07 (m, 3H), 2.07-1.99 (m, 3H), 1.98-1.85 (m, 9H), 0.96-0.84 (m, 3H). MS (ESI) m/z [M+H]⁺860.0.

### Example 1D

### Synthesis of Example 1D

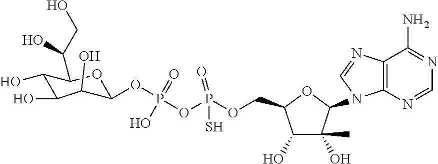 [SMILES: C[C@@]1(O)[C@H](O)[C@@H](COP(=O)(S)OP(=O)(O)OC2OC([C@@H](O)CO)C(O)C(O)C2O)O[C@H]1n1cnc2c(N)ncnc21]

A solution of Example 1C(5 mg, 0.0044 mmol) in 0.1M TEAB/MeOH/TEA=4:3:0.05 (1.5 mL) was stirred at 25° C. for 6 h. The solution was lyophilized to give the crude, then purified by prep-HLPC (0.1% FA in water/MeCN=98% to 95%) to give Example 1D (2.7 mg, 88.6%) as a white solid. ¹H NMR (400 MHz, D₂O) 8 8.59 (s, 1H), 8.22 (s, 1H), 6.10 (s, 1H), 5.18 (d, J=8.5 Hz, 1H), 4.41-4.34 (m, 1H), 4.31-4.23 (m, 2H), 4.20-4.13 (m, 1H), 4.04-3.98 (m, 1H), 3.84-3.80 (m, 1H), 3.72-3.66 (m, 1H), 3.67-3.55 (m, 3H), 3.28-3.23 (m, 1H), 0.86 (s, 3H). MS (ESI) m/z [M−H]⁻647.6.

### Example 1A

### Synthesis of Example 1A

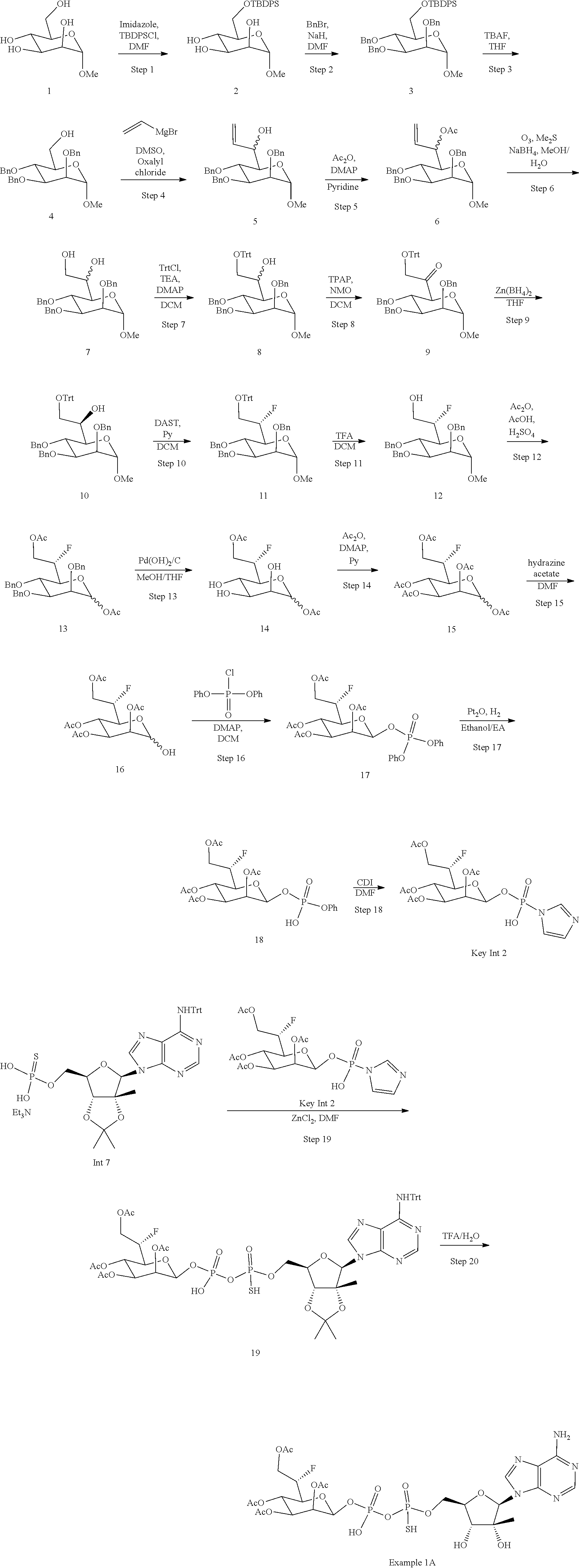

### Step 1: Synthesis of Compound 2

To a solution of compound 1 (450 g, 2.32 mol) in DMF (4.5 L) was added 1H-imidazole (346.7 g, 5.1 mol), followed by TBDPSCl (764 g, 2.78 mol) at 0° C. The mixture was stirred at rt overnight, the desired product was found based on LCMS, water (5 L) was added, and extracted with EA (5 L×2). The combined organic layer was dried over Na₂SO₄, and concentrated and purified with column chromatography (PE/EA=from 5/1 to 2/1) to give the title compound (700 g, 69%) as a colorless oil. ¹H NMR (400 MHz, MeOD) δ 7.80-7.72 (m, 4H), 7.46-7.37 (m, 6H), 4.70 (d, J=1.4 Hz, 1H), 4.05 (dd, J=10.8, 1.8 Hz, 1H), 3.87-3.81 (m, 2H), 3.70-3.64 (m, 2H), 3.61-3.54 (m, 1H), 3.43 (s, 3H), 1.05 (s, 9H).

### Step 2: Synthesis of Compound 3

To a mixture of compound 2 (700 g, 1.62 mol) and (bromomethyl)benzene (1.1 L, 9.72 mmol) in DMF (7 L) was added NaH (388.8 g, 60%) at 0° C. The mixture was stirred at rt overnight, the desired product was found based on LCMS, water (10 L) was added, and extracted with EA (1 L×2). The combined organic layer was washed with brine, dried, concentrated and purified with column chromatography (EA in PE 0˜9%) to give the title compound (700 g, 59.7%) as a colorless oil. ¹H NMR (400 MHz, MeOD) δ 7.72-7.64 (m, 4H), 7.41-7.37 (m, 4H), 7.35-7.24 (m, 13H), 7.21-7.18 (m, 2H), 7.12-7.10 (m, 2H), 4.85-4.78 (m, 2H), 4.73-4.66 (m, 2H), 4.60-4.52 (m, 3H), 4.04-3.96 (m, 1H), 3.87-3.80 (m, 4H), 3.59-3.53 (m, 1H), 3.31 (s, 3H), 1.01 (s, 9H).

### Step 3: Synthesis of Compound 4

To a solution of compound 3 (700 g, 0.99 mol) in THF (7 L) was added 1M TBAF (1.99 L, 1.99 mol). The mixture was stirred at rt overnight, monitored by TLC, concentrated and purified with column chromatography (EA in PE 0˜30%) to give the title compound (350 g, 71.87%) as a colorless oil. MS (ESI) m/z [M+Na]⁺486.8.

### Step 4: Synthesis of Compound 5

To a solution of oxalyl chloride (205 g, 1.6 mol) in THF (2.2 L) was added DMSO (252 g, 3.2 mol) in THF (540 mL) at −70° C., stirred at this temperature for 15 min, compound 4 (300 g, 0.65 mol) in THF (1.5 L) was added, stirred at −60° C. for 1h. Trimethylamine (654 g, 6.5 mmol) was added, the mixture was warmed to rt, stirred for 1h. Cooled to −70° C., Vinylmagnesium bromide (3.23 L, 3.23 mol) was added slowly, stirred for 2 h at −70° C. Saturated NH₄Cl (3000 mL) was added, extracted with EA (3 L×2). The combined organic layer was dried, concentrated and purified with column chromatography (PE/EA=10/1 to 5/1) to give the title compound (190 g, 56.97%) as a yellow oil. ¹H NMR (400 MHz, CDCl₃) δ 7.38-7.28 (m, 15H), 6.04-5.96 (m, 1H), 5.37 (d, J=17.2 Hz, 1H), 5.20 (d, J=10.5 Hz, 1H), 4.98 (d, J=10.8 Hz, 1H), 4.77-4.65 (m, 4H), 4.63 (s, 2H), 4.42 (d, J=2.7 Hz, 1H), 4.17-4.10 (m, 1H), 3.90 (dd, J=9.4, 3.0 Hz, 1H), 3.78 (dd, J=2.5, 2.1 Hz, 1H), 3.56 (dd, J=9.7, 1.3 Hz, 1H), 3.26 (s, 3H).

### Step 5: Synthesis of Compound 6

To a solution of compound 5 (220 g, 0.45 mol) in Pyridine (2.2 L) was added Ac₂O (91.5 g, 0.9 mol), followed by DMAP (5.5 g, 45 mmol). The mixture was stirred at rt overnight, the desired product was found based on LCMS. The reaction was concentrated and water (3 L) was added, extracted with EA (1.5 L×2). The combined organic layer was dried, concentrated and purified with column chromatography (PE/EA=4/1) to give the title compound (220 g, 87.5%) as a colorless oil. ¹H NMR (400 MHz, CDCl₃) δ 7.35-7.26 (m, 15H), 6.02-5.94 (m, 1H), 5.78 (dd, J=6.5, 1.3 Hz, 1H), 5.39-5.34 (m, 1H), 5.29-5.25 (m, 1H), 4.86 (dd, J=27.7, 5.8 Hz, 2H), 4.79-4.71 (m, 2H), 4.58 (s, 2H), 4.48 (d, J=10.0 Hz, 1H), 3.92-3.86 (m, 2H), 3.81-3.77 (m, 1H), 3.67 (dd, J=9.3, 1.7 Hz, 1H), 3.27 (s, 3H), 2.16 (s, 3H).

### Step 6: Synthesis of Compound 7

A mixture of compound 6 (22 g×10, 0.41 mol) in DCM/MeOH=1/1 (220 mL×10) was stirred at −78° C. for 40 min under O₃ atmosphere, then quenched by (CH₃) 2S, and the mixture was stirred at rt overnight. Then it was concentrated and dissolved in MeOH/H2O=2/1 (2.1 L), NaBH₄ (62.50 g, 1.65 mol) was added at 0° C., then warmed to rt, and stirred for 3 h. The mixture was concentrated and extracted with DCM (2 L×2). The combined organic layer was dried, concentrated and purified with column chromatography (PE/EA=2/1) to give the title compound (206 g, 95%) as a colorless oil. MS (ESI) m/z [M+Na]⁺512.2.

### Step 7: Synthesis of Compound 8

To a mixture of compound 7 (190 g, 0.38 mol) in DCM (2 L) was added TEA (78 g, 0.76 mmol) and DMAP (24 g, 0.19 mol), followed by TriphenylMethyl chloride (214 g, 0.76 mol). The mixture was stirred at 50° C. for 24 h. Then it was concentrated and purified with column chromatography (1% TEA in PE to PE/EA=10/1 to 4/1) to give the title compound (200 g, 67.1%) as a yellow solid. ¹H NMR (400 MHz, CDCl₃) δ 7.36-7.19 (m, 30H), 4.97 (d, J=11.0 Hz, 1H), 4.73 (dd, J=11.7, 5.7 Hz, 2H), 4.67-4.59 (m, 4H), 4.13-4.09 (m, 2H), 3.90 (dd, J=9.4, 3.1 Hz, 1H), 3.75-3.70 (m, 2H), 3.37 (dd, J=9.1, 6.4 Hz, 1H), 3.10-3.06 (m, 1H), 3.06 (s, 3H).

### Step 8: Synthesis of Compound 9

To a solution of compound 8 (200 g, 0.28 mol) in DCM (2 L) was added 4A molecular sieve (200 g) and NMO (158 g, 1.4 mol), stirred at rt for 0.5 h, TPAP (9.54 g, 0.028 mol) was added at 0° C., the mixture was stirred at rt for 2 h. Then it was filtered, concentrated and purified with column chromatography (1% TEA in PE/EA=20/1 to 5/1) to give the title compound (140 g, 70%) as a colorless oil. ¹H NMR (400 MHz, CDCl₃) δ 7.33-7.22 (m, 30H), 4.74-4.64 (m, 4H), 4.62-4.50 (m, 3H), 4.22 (d, J=8.5 Hz, 1H), 4.10-4.05 (m, 1H), 4.02-3.98 (m, 2H), 3.82 (dd, J=8.3, 3.0 Hz, 1H), 3.68 (t, J=2.9 Hz, 1H), 3.22 (s, 3H).

### Step 9: Synthesis of Compound 10

To a solution of compound 9 (140 g, 0.19 mol) in THF (1.4 L) was added Zn(BH₄)₂ (1 M, 209 mL) at 0° C., the mixture was stirred at 0° C. for 1 h, the desired product was found based on LCMS, water (1.5 L) was added, and extracted with EA (1 L×2). The combined organic layer was dried, concentrated and purified with column chromatography (EA in PE=0˜20%) to give the title compound (100 g, 70%) as a colorless oil. ¹H NMR (400 MHz, CDCl₃) δ 7.44-7.39 (m, 4H), 7.28-7.20 (m, 26H), 4.87 (d, J=10.8 Hz, 1H), 4.70-4.59 (m, 3H), 4.55 (d, J=1.7 Hz, 2H), 4.40 (d, J=10.8 Hz, 1H), 4.16-4.08 (m, 1H), 3.95-3.84 (m, 2H), 3.73-3.69 (m, 1H), 3.66 (dd, J=9.2, 4.1 Hz, 1H), 3.36 (dd, J=9.9, 7.0 Hz, 1H), 3.29-3.24 (m, 1H), 3.19 (s, 3H).

### Step 10: Synthesis of Compound 11

To a solution of compound 10 (100 g, 0.135 mol) in DCM (1 L) was added DAST (109 g, 0.67 mol) and Pyridine (106.6 g, 1.35 mmol) at 0° C. The mixture was stirred at rt overnight. Then it was concentrated and purified with column chromatography (EA in PE with 1% TEA=0˜80%) to give the title compound (50 g, 49.6%) as a colorless oil. MS (ESI) m/z [M+Na]⁺761.3.

### Step 11: Synthesis of Compound 12

To a solution of compound 11 (50 g, 67.6 mmol) in DCM (500 mL) was added TFA (100 mL). The mixture was stirred at rt for 1 h. Then it was concentrated and purified with column chromatography (PE/EA=10/1 to 1/1) to give the title compound (21 g, 62.5%) as a yellow oil. MS (ESI) m/z [M+H₂O+H]⁺514.2.

### Step 12: Synthesis of Compound 13

To a solution of compound 12 (21 g, 42.29 mmol) in AcOH (105 mL) and Ac₂O (105 mL) was added Con. H₂SO₄ (9.4 mL) at 0° C. The mixture was stirred at 0° C. for 1 h, monitored by LCMS, EA (200 mL) was added, poured into ice water, and extracted with EA (200 mL×2). The combined organic layer was washed with brine, dried, concentrated and purified with column chromatography (PE/EA=10/1 to 5/1) to give the title compound (12 g, 47%) as a colorless oil. MS (ESI) m/z [M+H₂O+H]⁺584.2.

### Step 13: Synthesis of Compound 14

To a solution of compound 13 (700 mg, 1.24 mmol) in MeOH/THF/H₂O/AcOH=10:5:1:0.25 (10 ml) was added Pd(OH) 2/C(700 mg). The mixture was stirred at 40° C. for 48 h under H₂ atmosphere. Then it was filtered and concentrated to give the title compound (500 mg, crude) as a colorless oil. MS (ESI) m/z [M+H₂O+H]⁺314.1.

### Step 14: Synthesis of Compound 15

To a solution of compound 14 (500 mg, 1.69 mmol) in pyridine (5 mL) was added Ac₂O (861 mg, 8.44 mmol) and DMAP (103 mg, 0.84 mmol). The mixture was stirred at rt for 30 min, water (10 mL) was added, and extracted with EA (10 mL×2). The combined organic layer was washed with brine, dried, concentrated and purified with Flash (12 g, EA in PE=0˜25%) to give the title compound (500 mg, 66%) as a yellow oil. ¹H NMR (400 MHz, CDCl₃) δ 6.10 (d, J=1.9 Hz, 1H), 5.60-5.50 (m, 1H), 5.39-5.31 (m, 1H), 5.26-5.24 (m, 1H), 4.75-4.55 (m, 1H), 4.44-4.35 (m, 1H), 4.34-4.22 (m, 1H), 4.01-3.92 (m, 1H), 2.20-2.16 (m, 6H), 2.09 (dd, J=6.6, 3.6 Hz, 6H), 2.01 (s, 3H).

### Step 15: Synthesis of Compound 16

To a solution of compound 15 (500 mg, 1.18 mmol) in DMF (5 mL) was added Hydrazine acetate (164 mg, 1.78 mmol). The mixture was stirred at rt for 30 min, water (10 mL) was added, and extracted with EA (10 mL×2). The combined organic layer was dried, concentrated and purified with Flash (EA in PE 0˜60%) to give the title compound (230 mg, 48%) as a yellow oil. ¹H NMR (400 MHz, CDCl₃) δ 5.52-5.47 (m, 1H), 5.44-5.39 (m, 1H), 5.30-5.22 (m, 2H), 4.78-4.60 (m, 1H), 4.43-4.25 (m, 2H), 4.11-4.04 (m, 1H), 2.16 (s, 3H), 2.10 (s, 3H), 2.06 (s, 3H), 1.99 (s, 3H).

### Step 16: Synthesis of Compound 17

To a mixture of compound 15 (230 mg, 0.6 mmol) and 4-Dimethylaminopyridine (362 mg, 2.96 mmol) in DCM (12 mL) was added diphenyl chlorophosphonate (481 mg, 1.79 mmol) in DCM (12 mL) over 30 min. Then the mixture was warmed to rt and stirred overnight. The reaction was concentrated and purified with Flash (EA in PE 0˜30%) to give the title compound (160 mg, 41%) as a colorless oil. MS (ESI) m/z [M+Na]⁺635.1.

### Step 17: Synthesis of Compound 18

To a solution of compound 17 (160 mg, 0.26 mmol) in EA/EtOH=1/1 (5 mL) was added PtO₂ (44 mg, 0.6 eq). The mixture was stirred at rt for 48 h under H₂ atmosphere. The resulting mixture was filtered and concentrated to give (2S,3S,5S,6S)-2-((S)-2-acetoxy-1-fluoroethyl)-6-(phosphonooxy)tetrahydro-2H-pyran-3,4,5-triyl triacetate (120 mg, crude) as a colorless oil, which was used in the next step without further purification.

### Step 18: Synthesis of key Int 2

To a solution of compound 18 (120 mg, 0.26 mmol) in DMF (1 mL) was added CDI (420 mg, 2.6 mmol). The mixture was stirred at rt for 1 h, the desired product was found based on LCMS, and MeOH (0.13 mL) was added slowly. The mixture was concentrated and used in the next step without further purification. MS (ESI) m/z [M−H]⁻509.0.

### Step 19 and 20: Synthesis of Example 1A

The title compound was obtained from key Int 2 with similar procedure to those described for Example 1C(24 mg, 50%) as a white solid. ¹H NMR (400 MHz, MeOD) δ 8.63 (d, J=34.0 Hz, 1H), 8.11 (s, 1H), 6.03 (s, 1H), 5.66 (t, J=9.5 Hz, 1H), 5.58 (dd, J=6.7, 3.1 Hz, 1H), 5.27 (t, J=10.0 Hz, 1H), 5.13 (dd, J=10.1, 3.2 Hz, 1H), 4.65 (d, J=8.4 Hz, 1H), 4.53-4.27 (m, 4H), 4.25-4.19 (m, 1H), 4.09 (d, J=6.7 Hz, 1H), 3.82-3.73 (m, 1H), 2.06 (d, J=2.3 Hz, 3H), 1.94 (dd, J=6.1, 3.7 Hz, 6H), 1.84 (s, 3H), 0.83 (d, J=5.6 Hz, 3H). MS (ESI) m/z [M+H]⁺820.0.

### Example 8C

### Synthesis of Example 8C

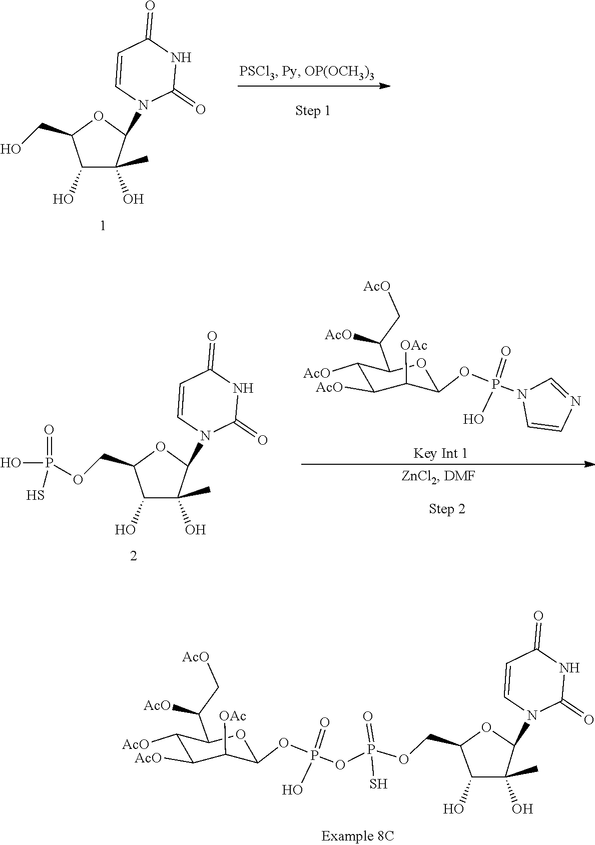 [SMILES: C[C@@]1(O)[C@H](O)[C@@H](CO)O[C@H]1n1ccc(=O)[nH]c1=O ; C[C@@]1(O)[C@H](O)[C@@H](COP(=O)(O)S)O[C@H]1n1ccc(=O)[nH]c1=O ; CC(=O)OC[C@H](OC(C)=O)C1OC(OP(=O)(O)n2ccnc2)C(OC(C)=O)C(OC(C)=O)C1OC(C)=O ; CC(=O)OC[C@H](OC(C)=O)C1OC(OP(=O)(O)OP(=O)(S)OC[C@H]2O[C@@H](n3ccc(=O)[nH]c3=O)[C@](C)(O)[C@@H]2O)C(OC(C)=O)C(OC(C)=O)C1OC(C)=O]

### Step 1: Synthesis of Compound 2

A solution of compound 1 (0.2 g, 0.78 mmol) in trimethyl phosphate (3 mL) was charged with argon for 3 times and cooled to 0° C. with ice-water. Pyridine (0.123 g, 1.56 mmol) and PSCl₃ (0.39 g, 2.34 mmol) were added successively. Then the mixture was stirred at 0° C. for 2 h. The mixture was quenched with H₂O, extracted with DCM, dried and concentrated. The crude product was purified by prep-HPLC(Daisogel-C18-5-100, 100% water, retention time: 10-20 min) followed by lyophilization to afford the desired product (0.2 g, 73%) as a light yellow solid. ¹H NMR (400 MHz, D₂O) 8=8.06 (d, J=8.1 Hz, 1H), 5.91 (s, 1H), 5.86 (d, J=8.1 Hz, 1H), 4.16-4.09 (m, 1H), 4.03 (d, J=9.3 Hz, 1H), 4.00-3.93 (m, 2H), 1.09 (s, 3H). MS (ESI) m/z [M−H]⁻353.0.

### Step 2: Synthesis of Example 8C

A solution of compound 2 (0.15 g, 0.42 mmol) and Key Int 1 (0.3 g crude, 0.42 mmol) in DMF (2 mL) was charged with argon for 3 times. ZnCl₂ (4.2 mL, 1M in THF, 4.2 mmol) was added dropwise and the mixture was stirred at rt for 16 h. The mixture was quenched with H₂O, purified by prep-HPLC(Daisogel-C18-5-100, 25% acetonitrile in water, retention time: 25-35 min) followed by lyophilization to afford the desired product (80 mg, 22.6%) as a white solid. ¹H NMR (400 MHz, D₂O) 8 7.94 (t, J=8.1 Hz, 1H), 5.95-5.82 (m, 2H), 5.51 (d, J=14.3 Hz, 2H), 5.27-5.12 (m, 2H), 5.00 (dd, J=19.9 Hz, 9.8, 1H), 4.36 (ddd, J=23.2 Hz, 15.8, 6.4, 2H), 4.26-4.09 (m, 2H), 4.08-3.98 (m, 2H), 3.90 (d, J=9.2 Hz, 1H), 2.13 (s, 3H), 2.04 (s, 3H), 1.95 (s, 6H), 1.87 (s, 3H), 1.09 (s, 3H). MS (ESI) m/z [M−H]⁻834.8.

### Example 59C

### Synthesis of Example 59C

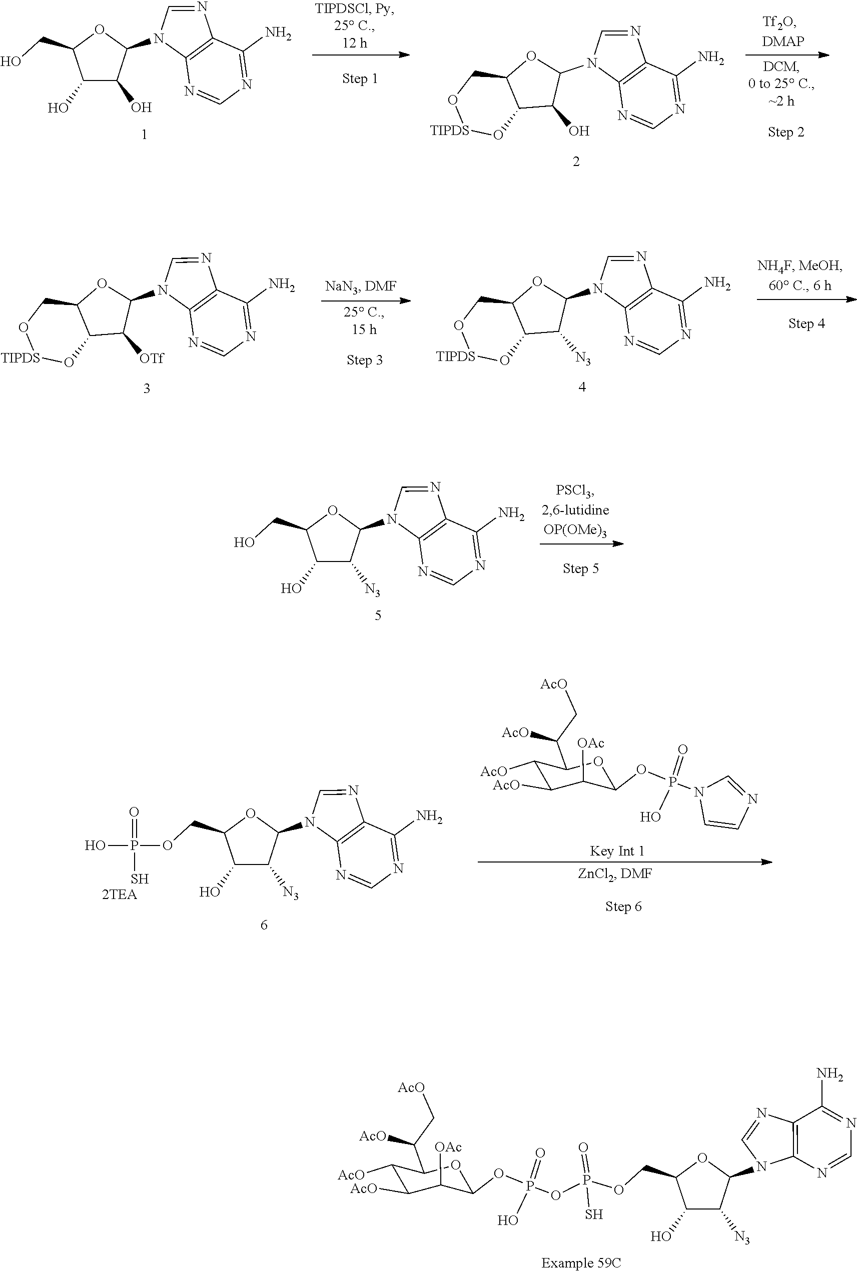

### Step 1: Synthesis of Compound 2

To a solution of compound 1 (44.0 g, 165 mmol) in Pyridine (300 mL) was added TIPDSCI (57.1 g, 181 mmol, 57.9 mL) at 25° C., the reaction mixture was stirred at 25° C. for 14 hrs. TLC(petroleum ether/ethyl acetate=1/2) showed consumption of starting material and one main new spot formed. The reaction mixture was diluted with Sat. NaHCO₃ (600 mL) and EtOAc (900 mL), the organic phase was separated, washed with water (300 mL×2) and brine (300 mL), dried over Na₂SO₄, filtered and concentrated under reduced pressure to afford the title compound (65.0 g, 72.9%) as a yellow solid. The crude product was used in the next step without further purification. ¹H NMR (400 MHz, CDCl₃) δ 8.59 (d, J=4.0 Hz, 1H), 8.18 (s, 1H), 8.11 (s, 1H), 6.20 (d, J=6.0 Hz, 1H), 6.09 (br s, 2H), 4.62 (d, J=7.6 Hz, 2H), 4.04 (d, J=3.2 Hz, 2H), 3.90-3.80 (m, 1H), 1.20-1.00 (m, 28H).

### Step 2: Synthesis of Compound 3

To a solution of compound 2 (56.5 g, 111 mmol) in DCM (600 mL) was added DMAP (40.6 g, 333 mmol) at −30 to −50° C., after stirred for 30 mins, then Tf₂O (39.1 g, 139 mmol, 22.9 mL) in DCM (100 mL) was added at −30 to −50° C., and the reaction mixture was stirred at 25° C. for 5.5 h. TLC(petroleum ether/ethyl acetate=1/2) showed one main new spot formed. The reaction mixture was washed with 20% of Citric acid solution (100 mL×5) and organic layer was washed with brine (50.0 mL), dried over Na₂SO₄, filtered and concentrated under reduced pressure to give the title compound (70.0 g, 69.8%) as a yellow oil. The crude product was used in the next step without further purification. ¹H NMR (400 MHz, CDCl₃) δ 8.33 (s, 1H), 7.93 (s, 1H), 6.40 (d, J=6.0 Hz, 1H), 5.69 (br s, 2H), 5.51-5.46 (m, 1H), 5.41 (d, J=7.2 Hz, 1H), 4.27-4.19 (m, 1H), 4.09 (dd, J=3.3, 12.3 Hz, 1H), 3.97 (br dd, J=3.6, 6.8 Hz, 1H), 1.12-1.04 (m, 28H).

### Step 3: Synthesis of Compound 4

To a solution of compound 3 (23.0 g, 35.8 mmol) in DMF (140 mL) was added NaN₃ (4.14 g, 63.7 mmol) at 25° C., then the reaction mixture was stirred at 25° C. for 28 hrs. TLC (petroleum ether/ethyl acetate=2/1) showed one main new spot formed. The reaction mixture was diluted with water (2.50 L) and EtOAc (1.00 L), extracted with EtOAc (600 mL×3). The combined organic layer was washed with brine (500 mL), dried over Na₂SO₄, filtered and concentrated under reduced pressure to give a residue. The residue was purified by column chromatography (SiO₂, petroleum ether/ethyl acetate=50/1 to 5/1, petroleum ether/ethyl acetate=2/1) to afford the title compound (25.6 g, 44.5%) as a white solid. ¹H NMR (400 MHz, DMSO-d₆) δ 8.22 (s, 1H), 8.06 (s, 1H), 7.38 (br s, 2H), 5.83 (d, J=1.2 Hz, 1H), 5.44 (dd, J=6.0, 8.4 Hz, 1H), 5.01 (dd, J=1.2, 6.0 Hz, 1H), 4.08-3.90 (m, 3H), 1.15-0.98 (m, 28H).

### Step 4: Synthesis of Compound 5

To a solution of compound 4 (25.6 g, 47.9 mmol) in MeOH (250 mL) was added NH₄F (21.3 g, 574 mmol) at 25° C., then the reaction mixture was stirred at 60° C. for 5 h. TLC (dichloromethane/methanol=10/1) showed one main new a pot formed. The reaction mixture was concentrated under reduced pressure to remove solvent. The crude product was triturated with petroleum ether (100 mL) at 25° C. for 1 h, the mixture was filtered and filter cake was triturated with water (100 mL) at 25° C. for 1 h, the mixture was filtered to give the title compound (10.5 g, 35.9 mmol, 75.0%) as a white solid. The crude product was used in the next step without further purification. ¹H NMR (400 MHz DMSO-d₆) δ 8.39 (s, 1H), 8.15 (s, 1H), 7.37 (s, 2H), 6.09-5.99 (m, 2H), 5.29 (t, J=5.6 Hz, 1H), 4.64 (t, J=5.6 Hz, 1H), 4.58-4.48 (m, 1H), 3.99 (q, J=3.6 Hz, 1H), 3.74-3.63 (m, 1H), 3.58 (br dd, J=4.0, 6.4 Hz, 1H).

### Step 5: Synthesis of Compound 6

To a solution of compound 5 (5.00 g, 17.1 mmol) and 2,6-dimethylpyridine (7.40 g, 68.4 mmol) in trimethyl phosphate (35.0 mL) was added PSCl₃ (5.80 g, 34.2 mmol) dropwise at 0° C. The reaction mixture was stirred at 25° C. for 4 h. LCMS showed one main product peak was formed. The reaction mixture was quenched with ice water (5.00 mL). The reaction mixture was adjusted to pH=6 with saturated NaHCO₃. The residue was purified by prep-HPLC(column: Nano 100*250*10 um; mobile phase: [H₂O-1M TEAB]; B %: 1%-100%,20 min) to give the title compound (1.70 g, containing 2 equiv. of TEA, 16.2%) as a white solid. ¹H NMR (400 MHz, D₂O) δ 8.64 (s, 1H), 8.16 (s, 1H), 6.08 (d, J=6.4 Hz, 1H), 4.80-4.73 (m, 2H), 4.32 (br s, 1H), 4.01 (br dd, J=2.4, 5.2 Hz, 2H). ³¹P NMR (162 MHz, D₂O) δ 43.37. MS (ESI) m/z [M−H]⁻387.0.

### Step 6: Synthesis of Example 59C

The title compound was obtained with similar procedures to those described for Example 8C(10.6 mg, 5%). ¹H NMR (400 MHz, D₂O) δ 8.75-8.72 (m, 1H), 8.45 (s, 1H), 6.23-6.21 (m, 1H), 5.68-5.60 (m, 2H), 5.32-5.26 (m, 2H), 5.15-5.09 (m, 1H), 4.48-4.40 (m, 2H), 4.37-4.24 (m, 3H), 4.17-4.12 (m, 1H), 2.21 (s, 3H), 2.15-2.14 (m, 3H), 2.06-2.05 (m, 3H), 2.03-2.01 (m, 3H), 1.97 (s, 3H). ³¹P NMR (162 MHz, D2O) δ 44.04, −15.22. MS (ESI) m/z [M−H]⁻868.8.

### Example 59A

### Synthesis of Example 59A

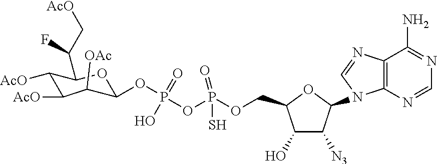 [SMILES: CC(=O)OC[C@H](F)C1OC(OP(=O)(O)OP(=O)(S)OC[C@H]2O[C@@H](n3cnc4c(N)ncnc43)[C@H](N=[N+]=[N-])[C@@H]2O)C(OC(C)=O)C(OC(C)=O)C1OC(C)=O]

The title compound was obtained from key Int 2 with similar procedures to those described for Example 59C(35 mg, 41.4%). ¹H NMR (400 MHz, D₂O) δ 8.71-8.68 (m, 1H), 8.41 (s, 1H), 6.17 (d, J=5.2 Hz, 1H), 5.65-5.54 (m, 2H), 5.35-5.22 (m, 2H), 4.90-4.85 (m, 1H), 4.81-4.77 (m, 2H), 4.46-4.33 (m, 3H), 4.26-4.14 (m, 2H), 4.08-3.94 (m, 1H), 2.16-2.15 (m, 3H), 2.08-2.03 (m, 6H), 1.95 (s, 3H). ³¹P NMR (162 MHz, D₂O) δ 43.71, −15.29. ¹⁹F NMR (376 MHz, D₂O) 8-206.38. MS (ESI) m/z [M−H]⁻828.8.

### Example 104C

### Synthesis of Example 104C

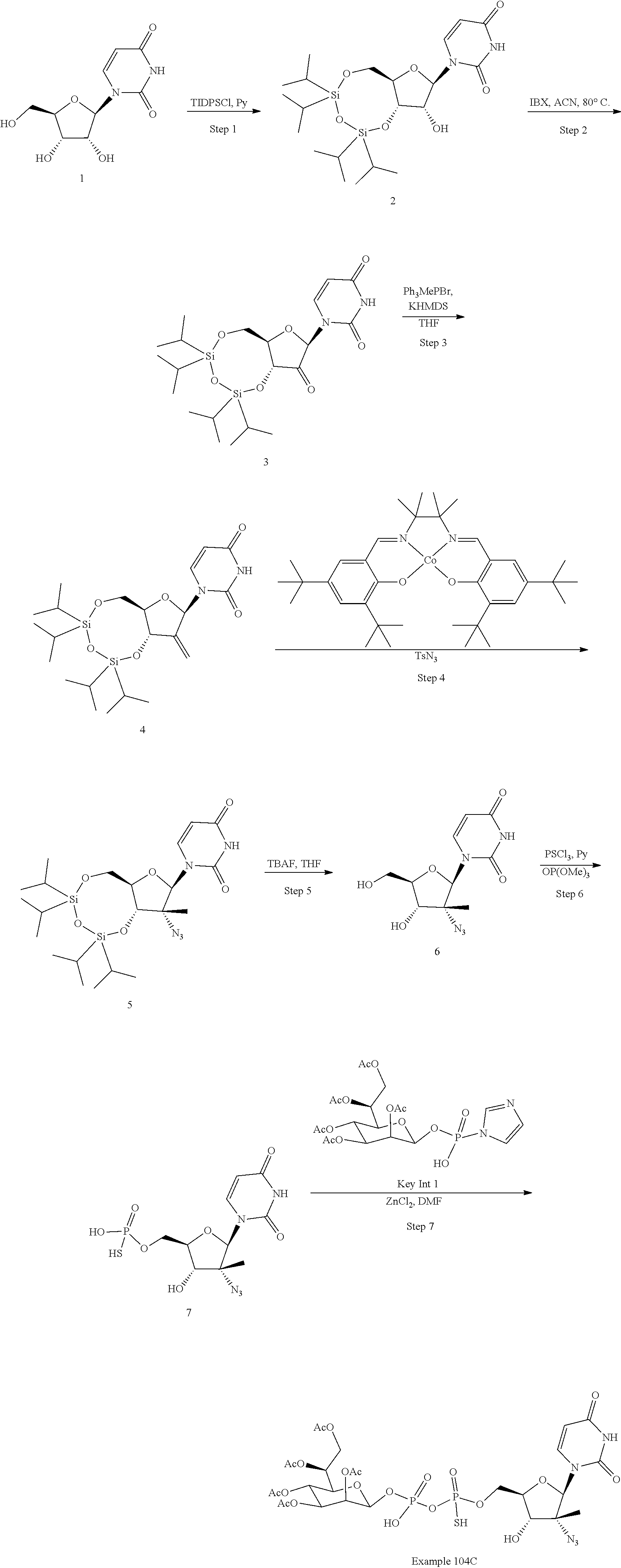

Compound 3 was obtained with the same procedures as described in Example 110C.

### Step 3: Synthesis of Compound 4

A solution of Methyltriphenylphosphonium bromide (3.57 g, 9.99 mmol) in THF (50 mL) was charged with N₂ for 3 times, potassium bis(trimethylsilyl) azanide (1 M, 9.99 mL) was added dropwise at 0° C. and stirred for 20 min, a solution of compound 3 (2.42 g, 4.99 mmol) in THF was added. The mixture was stirred at rt for 2 h, then at 35° C. for 3 h. Product was observed as main peak on LCMS. The mixture was quenched with aq. NH₄Cl, extracted with EA, the organic layer was isolated and dried over anhydrous Na₂SO₄. Filtered, concentrated, the residue was purified by silica-gel Combiflash (EA/PE=0˜40%) to give the title compound as white foam (1.2 g, 49.8%). MS (ESI) m/z [M+Na]⁺505.2.

### Step 4: Synthesis of Compound 5

To a solution of compound 4 (1 g, 2.07 mmol) in N-diazo-4-methyl-benzenesulfonamide (4.90 g, 24.86 mmol) was added 4,6,17,19-tetratert-butyl-11,11,12,12-tetramethyl-2,21-dioxa-10,13-diaza-1-cobaltapentacyclo[11.8.0.01,10.03,8.015,20]henicosa-3 (8),4,6,9,13,15 (20), 16,18-octaene (62.74 mg, 0.10 mmol). The mixture was stirred at rt for min, a solution of triethylsilane (1.20 g, 10.36 mmol, 1.65 mL) in Ethanol (5 mL) was added. The resulting mixture was stirred at 50° C. for 16 h. EA (50 mL) was added to the mixture, washed with aq. Na₂CO₃ (40 mL), water (50 mL) and brine (50 mL), dried over anhydrous Na₂SO₄, filtered and concentrated. The crude product was purified by silica gel CombiFlash (EA/PE=0˜50%) to give the title compound as yellow solid (235 mg, 21.6%). MS (ESI) m/z [M+H]⁺526.3

### Step 5: Synthesis of Compound 6

Into 40 mL vial was evacuated and flushed three times with nitrogen, added compound (200 mg, 0.38 mmol), TBAF (298.39 mg, 1.14 mmol, 330.44 μL) and THF (1.83 mL). The resulting solution was stirred at 25° C. for 1 h. The resulting solution was concentrated under reduced pressure and the residue was purified by flash chromatography on silica gel (MeOH/EA=0˜25%) to give the title compound (90 mg, 83.5%) as white solid. MS (ESI) m/z [M+H]⁺284.0.

### Step 6 and 7: Synthesis of Example 104C

The title compound was obtained with similar procedures to those described for Example 8C(32 mg, 11.7% for two steps). ¹H NMR (400 MHz, D₂O) δ 7.96 (d, J=8.1Z Hz, 1H), 5.91 (s, 2H), 5.58-5.47 (m, 2H), 5.24-5.16 (m, 2H), 5.03 (t, J=9.9 Hz, 1H), 4.42-4.35 (m, 1H), 4.34-4.28 (m, 1H), 4.27-4.21 (m, 1H), 4.18-4.12 (m, 1H), 4.09-4.02 (m, 3H), 2.15 (s, 3H), 2.05 (s, 3H), 1.97 (s, 3H), 1.95-1.94 (m, 3H), 1.90 (s, 3H), 1.31 (s, 3H). MS (ESI) m/z [M−H]⁻860.0.

### Example 21C

### Synthesis of Example 21C

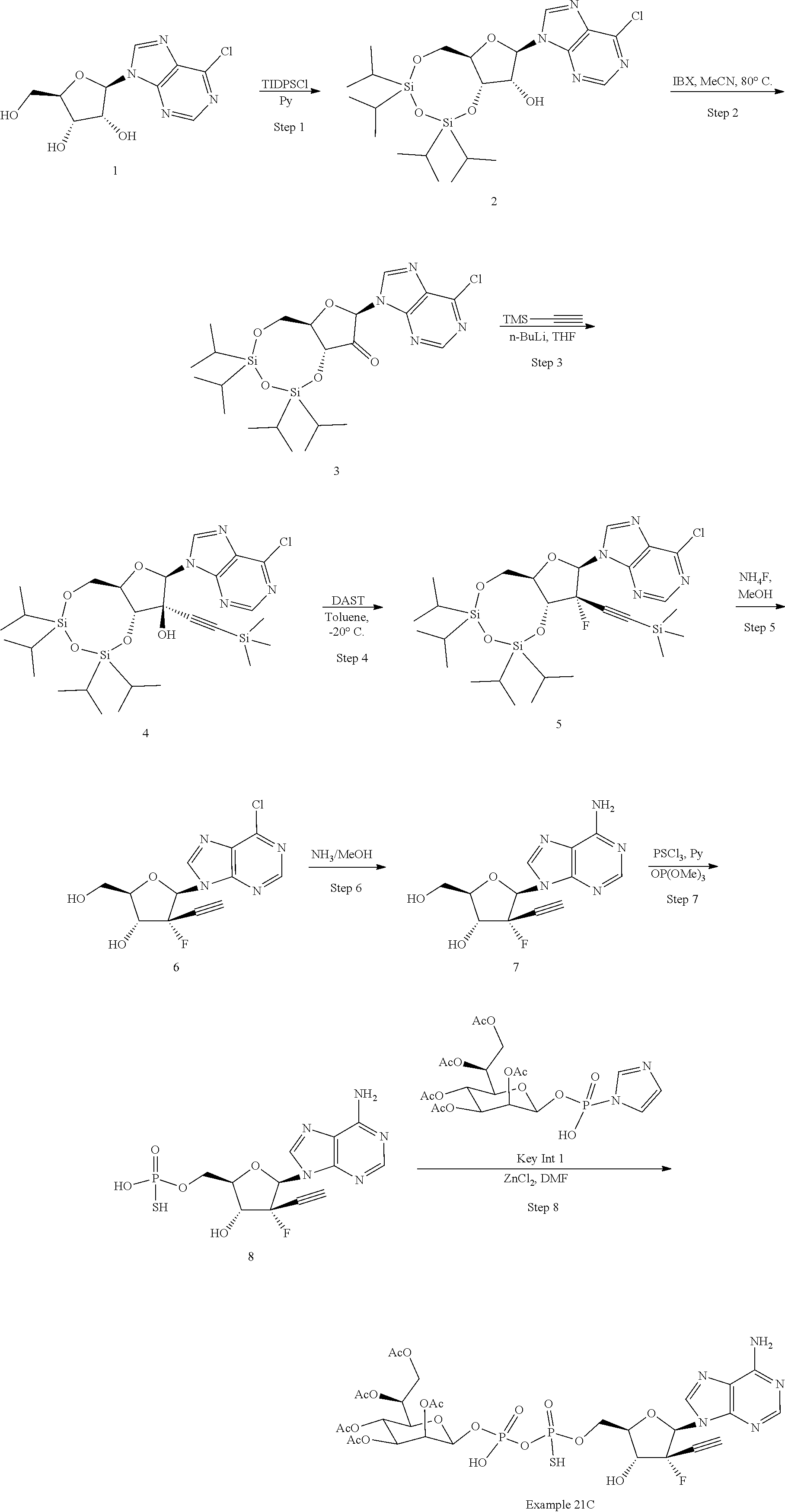

### Step 1: Synthesis of Compound 2

To a solution of compound 1 (5 g, 17.44 mmol) in Pyridine (50 mL) was added 1,3-Dichloro-1,1,3,3-tetraisopropyldisiloxane (5.5 g, 17.44 mmol), the mixture was stirred at rt for 4h, desired product was found based on LCMS. Then the solvent was removed in vacuo. The crude product was purified by silica gel column chromatography (EtOAc/PE=0˜60%) to give the title compound (5 g, 54%) as an off-white solid. MS (ESI) m/z [M+H]⁺528.9.

### Step 2: Synthesis of Compound 3

To a solution of compound 2 (5 g, 9.45 mmol) in MeCN (50 mL) was added IBX (5.82 g, 20.66 mmol), then the mixture was stirred at 80° C. for 5h, TLC(PE/EA=3/1) showed complete conversion. Filtered and the filtrate was concentrated to give the title compound (4.5 g, 90%) as a white solid. MS (ESI) m/z [M+H]⁺526.7.

### Step 3: Synthesis of Compound 4

To a solution of Trimethylsilylacetylene (4.2 g, 42.8 mmol) in THF (75 mL) was added n-BuLi (17.8 mL, 2.4 M in hexane) at −78° C., the solution was stirred at −78° C. for 30 min and then was warmed to −55° C. and stirred for 20 min, then cooled to −78° C., compound 3 (4.5 g, 8.56 mmol) in THF (20 mL) was added, the mixture was stirred at −78° C. for 1 h, then warmed to −30° C. and stirred for 2h, monitored by TLC until complete conversion of the starting material. The reaction was cooled to −78° C., saturated NH₄Cl (100 mL) was added slowly, extracted with EA (100 mL×2), the combined organic layers was washed with brine, dried over anhydrous Na₂SO₄, filtered and concentrated, the residue was purified with CombiFlash (silica gel 40 g, EA/PE=0˜12%) to give the title compound (3 g, 85%) as an off-white solid. ¹H NMR (400 MHz, CDCl₃) δ 8.73 (s, 1H), 8.54 (s, 1H), 6.35 (s, 1H), 4.45 (d, J=7.5 Hz, 1H), 4.17-4.05 (m, 3H), 1.17-1.03 (m, 28H), 0.20-0.16 (m, 9H). MS (ESI) m/z [M+H]⁺625.2.

### Step 4: Synthesis of Compound 5

To a solution of compound 4 (3 g, 4.8 mmol) in toluene (60 mL) was added DAST (4.6 g, 28.4 mmol) at −20° C., then the mixture was stirred at rt for 1.5 h, TLC showed complete conversion. EA (60 mL) was added, the mixture was poured into NaHCO₃ (60 mL), stirred for 5 min, extracted with EA (60 mL×2), the combined organic layer was washed with brine, dried over anhydrous Na₂SO₄, filtered and concentrated, and the residue was purified with CombiFlash (40 g, EA/PE=0˜10%) to give the title compound (1.6 g, 53%) as a yellow oil. MS (ESI) m/z [M+H]⁺626.8.

### Step 5: Synthesis of Compound 6

To a solution of compound 5 (1.6 g, 2.56 mmol) in MeOH (12 mL) was added NH₄F (1.2 g, 33.28 mmol), the mixture was stirred at 70° C. for 2h, desired product was found based on LCMS, concentrated and the residue was purified with CombiFlash (24 g, MeOH/DCM=0˜9%) to give the title compound (700 mg, 87%) as a yellow solid. MS (ESI) m/z [M+H]⁺312.8.

### Step 6: Synthesis of Compound 7

A solution of compound 6 (760 mg, 2.43 mmol) in 7M NH₃ in MeOH (15 mL) was stirred at 90° C. for 4h in a sealed tube, desired product was found based on LCMS, concentrated and the residue was purified with CombiFlash (4 g, MeOH/DCM=0˜15%) to give the title compound (200 mg, 32%) as an off-white solid. ¹H NMR (400 MHz, DMSO-d₆) δ 8.39 (s, 1H), 8.17 (d, J=5.4 Hz, 1H), 7.37 (s, 2H), 6.34 (d, J=17.1 Hz, 1H), 6.22 (d, J=7.6 Hz, 1H), 5.31 (t, J=5.3 Hz, 1H), 4.73-4.59 (m, 1H), 3.96 (d, J=9.2 Hz, 1H), 3.85 (ddd, J=12.4, 4.8, 1.9 Hz, 1H), 3.77 (d, J=5.4 Hz, 1H), 3.75-3.68 (m, 1H). ¹⁹F NMR (376 MHz, DMSO-d₆) δ-158.34. MS (ESI) m/z [M+H]⁺294.1.

### Steps 7 and 8: Synthesis of Example 21C

The title compound was obtained with similar procedures to those described for Example 8C(42.5 mg, 14% for two steps). ¹H NMR (400 MHz, D₂O) δ 8.63-8.60 (m, 1H), 8.37 (s, 1H), 6.49-6.44 (m, 1H), 5.57-5.52 (m, 2H), 5.21-5.15 (m, 2H), 5.02 (t, J=10.0 Hz, 1H), 4.46-4.33 (m, 2H), 4.33-4.18 (m, 3H), 4.06-4.02 (m, 1H), 2.95-2.93 (m, 1H), 2.13 (s, 3H), 2.05 (s, 3H), 1.95 (s, 3H), 1.91-1.90 (m, 3H), 1.87 (s, 3H). ³¹P NMR (162 MHz, D₂O) δ 44.07,43.69;-15.13, −15.29. ¹⁹F NMR (376 MHz, D₂O) δ-159.90, −160.05. MS (ESI) m/z [M−H]⁻828.1.

### Example 21D

### Synthesis of Example 21D

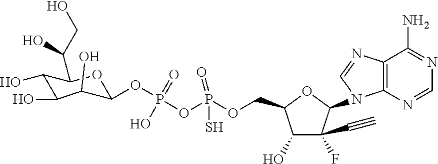 [SMILES: C#C[C@@]1(F)[C@H](O)[C@@H](COP(=O)(S)OP(=O)(O)OC2OC([C@@H](O)CO)C(O)C(O)C2O)O[C@H]1n1cnc2c(N)ncnc21]

The title compound was obtained from Example 21C with similar procedures to those described for Example 1D (1.6 mg, 10.8%). ¹H NMR (400 MHz, D₂O) δ 8.84-8.73 (m, 1H), 8.50 (s, 1H), 6.67-6.62 (m, 1H), 5.41-5.31 (m, 1H), 5.03-4.88 (m, 1H), 4.59-4.52 (m, 1H), 4.47-4.39 (m, 2H), 4.18-4.15 (m, 1H), 3.98 (t, J=6.5 Hz, 1H), 3.90-3.66 (m, 4H), 3.42-3.39 (m, 1H), 3.11-3.08 (m, 1H). ¹⁹F NMR (376 MHz, D₂O) δ-159.66. ³¹P NMR (162 MHz, D₂O) δ 43.41, −14.22. MS (ESI) m/z [M−H]⁻660.0.

### Example 22C

### Synthesis of Example 22C

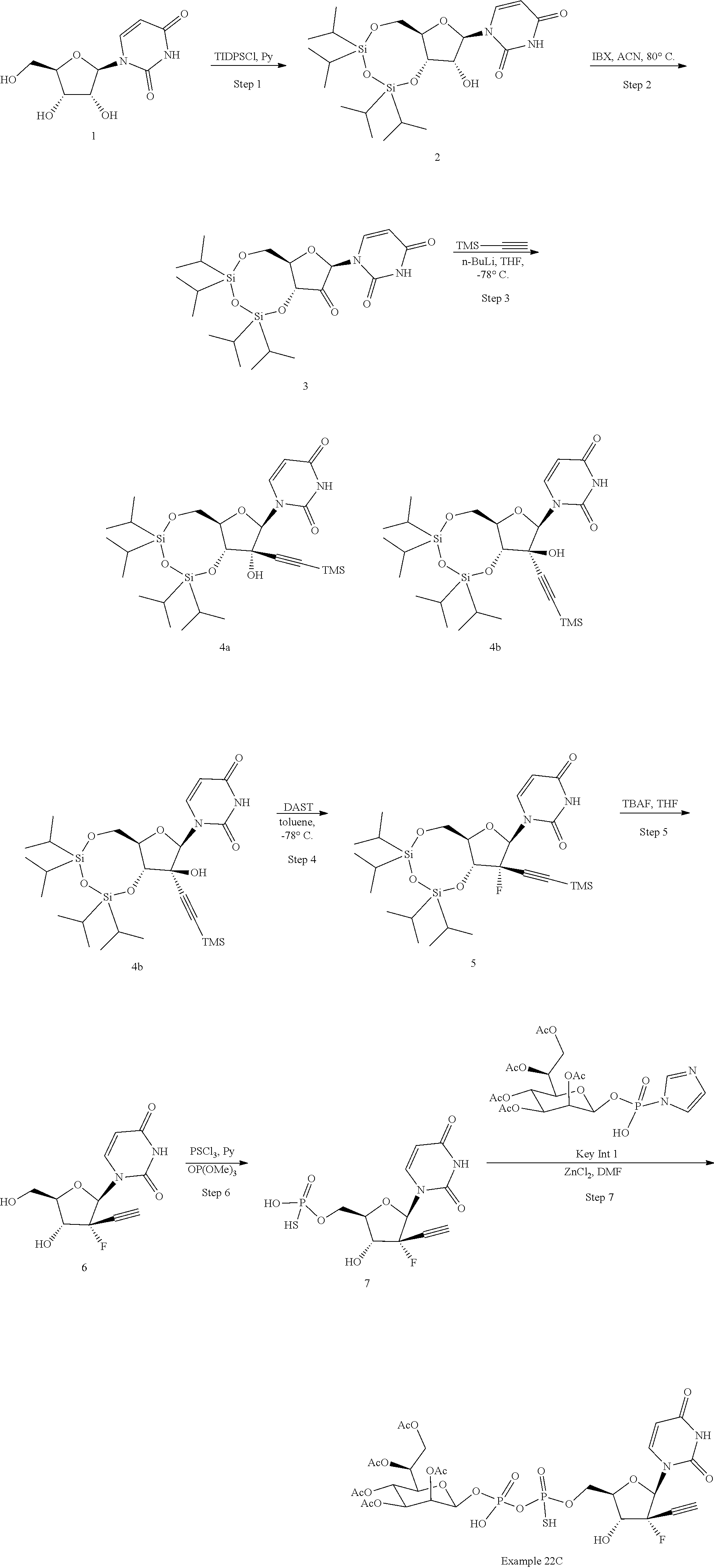

### Step 1: Synthesis of Compound 2

To a solution of compound 1 (25 g, 0.102 mol) in dry Pyridine (100 mL) was added 1,3-dichloro-1,1,3,3-tetraisopropyldisoxane (32.3 g, 0.102 mol). The resulting solution was stirred at rt for 4 h. Then the solvent was removed in vacuo. The crude product was purified by silica gel column chromatography (EA/PE=0˜40%) to give the title compound (33 g, 62.8%) as a white solid. MS (ESI) m/z [M+H]⁺486.9.

### Step 2: Synthesis of Compound 3

To a solution of compound 2 (33 g, 67.8 mmol) in dry ACN (160 ml) was added IBX (37.9 g, 135.6 mmol). The resulting solution was stirred at 80° C. for 5 h. Cooled to r.t., the solid was filtered out. The filtrate was concentrated under vacuum to give the title compound (33g crude) which was used for the next step without further purification. MS (ESI) m/z [M+H]⁺485.0.

### Step 3: Synthesis of Compound 4a and Compound 4b

To a solution of ethynyltrimethylsilane (10.1 g, 20.8 mmol) in THF (100 mL) was added n-BuLi (43 mL, 2.4M) at −78° C., stirred for 30 min at −78° C., then stirred for 30 min at −55° C. A solution of compound 3 (10 g, 20.63 mmol) in THF (40 mL) was added dropwise, stirred for 2 h. Sat. aqueous NH₄Cl (200 mL) was added to the mixture and extracted with EA (300 mL). The organic layer was dried over anhydrous Na₂SO₄, filtered, the filtrate was concentrated and purified by flash chromatography (EA/PE=0˜60%) to give compound 4a (400 mg, 3.3%) as a brown oil and compound 4b (5 g, 41.2%) as off-white solid. MS (ESI) m/z [M+H]⁺583.3.

### Step 4: Synthesis of Compound 5

A solution of compound 4b (0.5 g, 0.86 mmol) in Toluene (9.73 mL) was charged with N2 for 3 times, then cooled to −78° C. DAST (414.79 mg, 2.57 mmol) was then added dropwise and stirred for 2 h. The mixture was quenched with aq. Na₂CO₃ (30 mL) at −78° C., then extracted with EA (50 mL), the organic layer was dried over anhydrous Na₂SO₄, filtered and concentrated. The crude product was purified by silica-gel CombiFlash (EA/PE=0˜40%) to give the title compound as yellow oil (0.4 g, 79.7%). MS (ESI) m/z [M+H]⁺585.3, [M+Na]⁺607.3.

### Step 5: Synthesis of Compound 6

To a solution of compound 5 (0.4 g, 0.68 mmol) in THF was added Tetrabutylammonium fluoride trihydrate (431.5 mg, 1.37 mmol). The mixture was stirred at r.t. for 3 h and then was concentrated and the residue was purified by silica gel column (MeOH/EA=1˜10%) to give the title compound (0.1 g, 54.11%) as yellow oil. MS (ESI) m/z [M+H]⁺271.1, [M+Na]⁺293.1.

### Step 6 and 7: Synthesis of Example 22C

The title compound was obtained with similar procedures to those described for Example 8C(35 mg, 11% for two steps). ¹H NMR (400 MHz, D₂O) δ 7.87-7.85 (m, 1H), 6.19-6.15 (m, 1H), 5.87-5.85 (m, 1H), 5.51-5.47 (m, 2H), 5.19-5.11 (m, 2H), 5.01-4.96 (m, 1H), 4.38-4.26 (m, 3H), 4.22-4.17 (m, 1H), 4.16-4.06 (m, 2H), 4.00-3.98 (m, 1H), 3.25-3.24 (m, 1H), 2.09 (s, 3H), 2.01 (s, 3H), 1.93-1.89 (m, 6H), 1.85 (s, 3H). MS (ESI) m/z [M−H]⁻847.1.

### Example 22D

### Synthesis of Example 22D

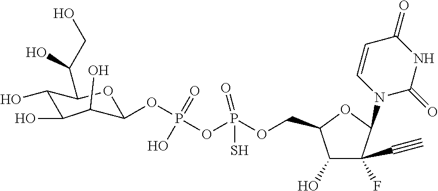 [SMILES: C#C[C@@]1(F)[C@H](O)[C@@H](COP(=O)(S)OP(=O)(O)OC2OC([C@@H](O)CO)C(O)C(O)C2O)O[C@H]1n1ccc(=O)[nH]c1=O]

The title compound was obtained from Example 22C with similar procedures to those described for Example 1D (1 mg, 8%). ¹H NMR (400 MHz, D₂O) δ 7.87-7.85 (m, 1H), 6.20-6.16 (m, 1H), 5.87-5.85 (m, 1H), 5.15-5.14 (m, 1H), 4.40-4.32 (m, 2H), 4.17-4.11 (m, 2H), 4.00-3.98 (m, 1H), 3.81-3.79 (m, 1H), 3.69-3.53 (m, 4H), 3.26-3.23 (m, 2H). MS (ESI) m/z [M−H]⁻637.1.

### Example 25C

### Synthesis of Example 25C

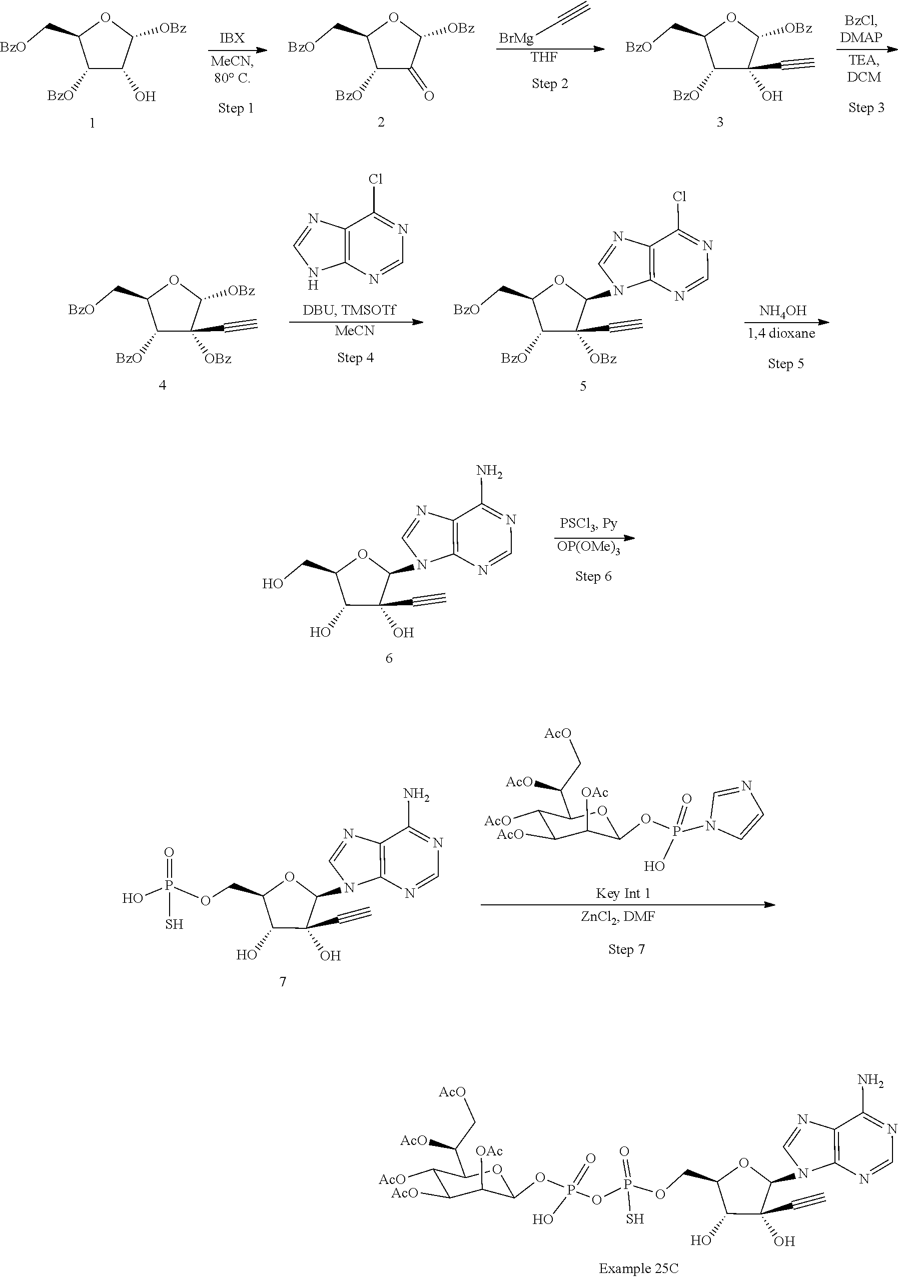

### Step 1: Synthesis of Compound 2

To a solution of compound 1 (30 g, 65 mmol) in ACN (300 mL) was added IBX (36.35 g, 0.13 mol). The reaction was stirred at 80° C. for 3 h. Then it was cooled to rt, filtered and the filtrate was concentrated without purification to give the title compound (27 g, 90.3%) as a yellow oil. ¹H NMR (400 MHz, MeOD) δ 8.18-8.03 (m, 6H), 7.66-7.55 (m, 3H), 7.52-7.36 (m, 6H), 6.40 (s, 1H), 5.37-5.29 (m, 1H), 4.74-4.66 (m, 1H), 4.65-4.52 (m, 2H). MS (ESI) m z [M+H₂O+Na]⁺500.8.

### Step 2: Synthesis of Compound 3

To a solution of compound 2 (12 g, 26 mmol) in THF (120 mL) was added bromo (ethynyl)magnesium (188 mL, 94 mmol) at −78° C. The solution was stirred at −78° C. for 0.5 h, and then poured into an ice-cold saturated NH₄Cl solution (300 mL). After extraction with EtOAc (100 mL×3), the combined organics were died over anhydrous Na₂SO₄, filtered and the filtrate was concentrated under reduced pressure to give the crude title compound as a yellow oil (12 g). MS (ESI) m/z [M+Na]⁺508.8.

### Step 3: Synthesis of Compound 4

To a solution of compound 3 (11.5 g, 23.6 mmol) in DCM (120 mL) was added 4-Dimethylaminopyridine (5.77 g, 47.2 mmol), TEA (7.15 g, 70.8 mmol) and BzCl (9.95 g, 70.8 mmol) at 0° C. The solution was stirred for 16 h at 25° C., then diluted with brine and carefully quenched with saturated NaHCO₃ (80 mL). After extraction with EtOAc (100 mL×3), the combined organics were died over Na₂SO₄, evaporated to give the crude and purified with column chromatography (EA/PE=0˜60%) to give the title compound (8.6 g, 55.5%) as a yellow oil. ¹H NMR (400 MHz, CDCl₃) δ 8.20-8.08 (m, 5H), 8.04-7.95 (m, 2H), 7.93-7.88 (m, 1H), 7.62-7.39 (m, 9H), 7.35-7.25 (m, 2H), 7.22-7.10 (m, 2H), 6.47-5.95 (m, 1H), 5.05-4.82 (m, 1H), 4.80-4.60 (m, 2H), 4.60-4.42 (m, 1H), 2.78-2.71 (m, 1H). MS (ESI) m/z [M+Na]⁺612.7.

### Step 4: Synthesis of Compound 5

To a solution of compound 4 (8.6 g, 14.56 mmol) and 6-chloro-9H-purine-(4.5 g, 29.12 mmol) in ACN (90 mL) was added DBU (12.4 g, 81.5 mmol) at 0° C. and stirred for 15 min. To this solution was added TMSOTf (25.9 g, 116.5 mmol) at 0° C. The solution was stirred for 15 mins at 0° C. and then 16 h at 70° C. The resulting reaction was diluted with brine and carefully quenched with saturated NaHCO₃ (100 mL). After extraction with EtOAc (100 mL×3), the combined organics were died over Na₂SO₄, evaporated to give the crude and purified with column chromatography (EA/PE=0˜70%) to give the title compound (2.8 g, 27.8%) as a yellow solid. MS (ESI) m/z [M+H]⁺623.0.

### Step 5: Synthesis of Compound 6

To a solution of compound 5 (2 g, 3.21 mmol) in dioxane (20 mL) was added NH₃H₂O 2O (60 mL). The mixture was stirred at 110° C. in sealed tube overnight. Then it was concentrated to give the crude. The residue was applied onto a silica gel column With DCM/MeOH (10:1) to give the title compound (760 mg, 73%) as a brown solid. ¹H NMR (400 MHz, MeOD) δ 8.48 (s, 1H), 8.17 (s, 1H), 6.16 (s, 1H), 4.57 (d, J=8.8 Hz, 1H), 4.09-3.94 (m, 2H), 3.86-3.77 (m, 1H), 2.65 (s, 1H). MS (ESI) m/z [M+H]⁺292.1.

### Steps 6 and 7: Synthesis of Example 25C

The title compound was obtained with similar procedures to those described for Example 8C(11 mg, 4% for two steps). ¹H NMR (400 MHz, D₂O) δ 8.65 (s, 1H), 8.36 (s, 1H), 6.17 (s, 1H), 5.56 (d, J=10.8 Hz, 2H), 5.27-5.17 (m, 2H), 5.03 (t, J=9.9 Hz, 1H), 4.53 (d, J=8.5 Hz, 1H), 4.42-4.36 (m, 2H), 4.28-4.20 (m, 3H), 4.09-4.04 (m, 1H), 2.58 (d, J=4.3 Hz, 1H), 2.14 (s, 3H), 2.05 (s, 3H), 1.96 (s, 3H), 1.92 (s, 3H), 1.89 (s, 3H). ³¹P NMR (162 MHz, D₂O) δ 44.00, −14.81. MS (ESI) m/z [M−H]⁻867.8.

### Example 25D

### Synthesis of Example 25D

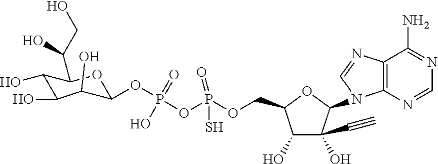 [SMILES: C#C[C@@]1(O)[C@H](O)[C@@H](COP(=O)(S)OP(=O)(O)OC2OC([C@@H](O)CO)C(O)C(O)C2O)O[C@H]1n1cnc2c(N)ncnc21]

The title compound was obtained from Example 25C with similar procedures to those described for Example 1D (1.58 mg, 10.4%). ¹H NMR (400 MHz, D₂O) δ 8.66 (s, 1H), 8.34 (s, 1H), 6.21 (s, 1H), 5.20 (d, J=8.4 Hz, 1H), 4.58 (d, J=8.5 Hz, 1H), 4.40-4.36 (m, 1H), 4.31-4.22 (m, 2H), 4.04 (s, 1H), 3.84 (t, J=6.5 Hz, 1H), 3.78-3.64 (m, 2H), 3.64-3.54 (m, 2H), 3.29-3.26 (m, 1H), 3.09 (q, J=7.3 Hz, 5H), 2.58 (s, 1H), 1.17 (t, J=7.3 Hz, 7H). ³¹P NMR (162 MHz, D₂O) δ 43.77, −14.25. MS (ESI) m/z [M−H]⁻658.0.

### Example 26C

### Synthesis of Example 26C

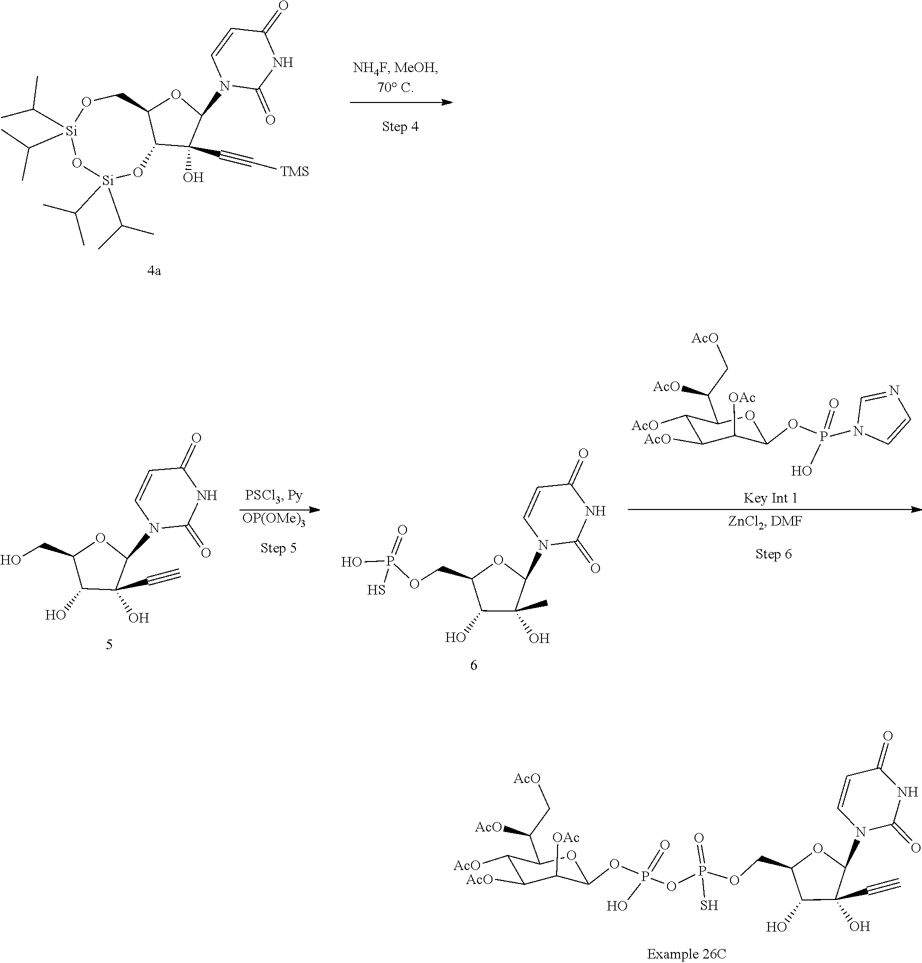

The starting material compound 4a was isolated in step 3 of Example 22C.

### Step 4: Synthesis of Compound 5

To a solution of compound 4a (400 mg, 0.69 mmol) in MeOH (10 mL) was added Ammonium fluoride (254 mg, 6.9 mmol). The resulting solution was stirred at 70° C. for 3 h. Then the solvent was removed in vacuo. The crude product was purified by silica gel column chromatography (MeOH/DCM 0˜10%) to give the title compound (139 mg, 74.2%) as an off-white solid. ¹H NMR (400 MHz, DMSO-d₆) δ 11.31 (s, 1H), 7.67 (d, J=8.2 Hz, 1H), 6.44 (s, 1H), 6.10 (s, 1H), 5.85 (d, J=5.7 Hz, 1H), 5.59 (d, J=8.2 Hz, 1H), 5.10 (t, J=5.4 Hz, 1H), 3.88-3.83 (m, 1H), 3.79-3.73 (m, 1H), 3.66-3.54 (m, 3H). MS (ESI) m/z [M+H]⁺268.9.

### Steps 5 and 6: Synthesis of Example 26C

The title compound was obtained with similar procedures to those described for Example 8C(7.5 mg, 1.7% for two steps). ¹H NMR (400 MHz, D₂O) δ 7.97-7.93 (m, 1H), 5.93-5.92 (m, 1H), 5.85-5.83 (m, 1H), 5.52-5.47 (m, 2H), 5.17-5.12 (m, 2H), 5.00-4.95 (m, 1H), 4.39-4.33 (m, 1H), 4.32-4.25 (m, 1H), 4.20-4.17 (m, 2H), 4.13-4.02 (m, 2H), 4.00-3.96 (m, 1H), 2.88-2.87 (m, 1H), 2.10-2.09 (m, 3H), 2.01-2.00 (m, 3H), 1.92-1.91 (m, 3H), 1.90-1.89 (m, 3H), 1.85-1.84 (m, 3H). ³¹P NMR (162 MHz, D₂O) δ 44.11, −14.63. MS (ESI) m z [M−H]⁻844.9.

### Example 26E

### Synthesis of Example 26E

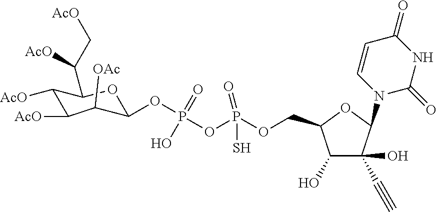 [SMILES: C#C[C@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1ccc(=O)[nH]c1=O]

The starting material compound 4b was isolated in step 3 of Example 22C, and the title compound was obtained by following the same procedures as described in Example 26C(9 mg, 8.8%). ¹H NMR (400 MHz, D₂O) δ 7.92 (d, J=8.0 Hz, 1H), 6.21 (s, 1H), 5.83 (d, J=8.1 Hz, 1H), 5.53-5.47 (m, 2H), 5.23-5.14 (m, 2H), 5.02 (t, J=10.0 Hz, 1H), 4.44-4.35 (m, 1H), 4.28-4.11 (m, 4H), 4.08-4.03 (m, 1H), 3.98 (d, J=10.0 Hz, 1H), 3.04 (s, 1H), 2.14 (s, 3H), 2.05 (s, 3H), 1.97-1.92 (m, 6H), 1.89 (s, 3H). MS (ESI) m/z [M−H]⁻845.0.

### Example 31C

### Synthesis of Example 31C

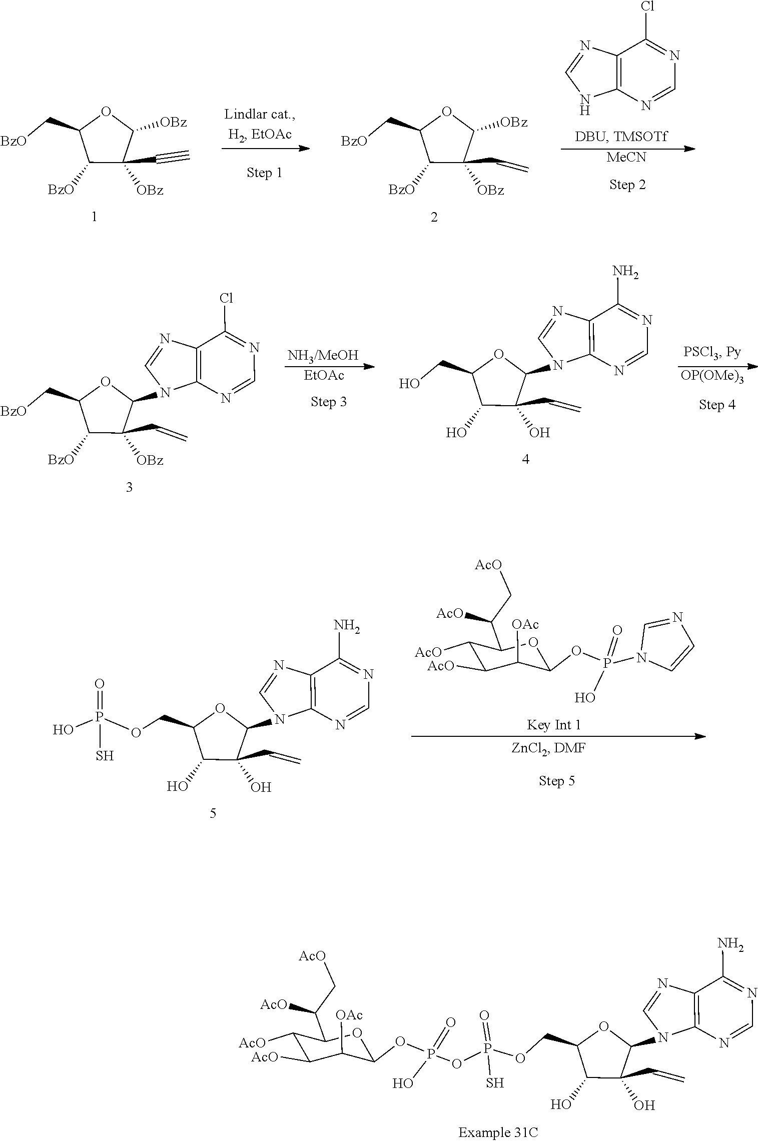

### Step 1: Synthesis of Compound 2

To a solution of compound 1 (obtained from step 3 in Example 25C, 1 g, 1.6 mmol) in EtOAc (10 mL), Lindlar catalyst (20%, 20 mg) was added. The mixture was stirred vigorously for 2 h under H₂ atmosphere at room temperature, then quenched by filtering the catalyst. The organic filtrate was concentrated under reduced pressure affording the title compound (900 mg, 89%) which was used for the next step directly without further purification. MS (ESI) m/z [M+Na]⁺615.0.

### Step 2: Synthesis of Compound 3

To a solution of compound 2 (900 mg, 1.5 mmol) and 6-chloro-9H-purine (255 mg, 1.65 mmol) in ACN (10 mL), DBU (770 mg, 5 mmol), TMSOTf (1.3 g, 5.8 mmol) were added successively, the crude mixture was stirred vigorously at 80° C. for 2 h, then the reaction was cooled to r.t. and quenched by adding saturated NaHCO₃ solution (15 mL), the mixture was washed with water (30 mL) and brine (30 mL), dried with Na₂SO₄, filtered and concentrated under reduced pressure, purified by chromatography (EA/PE=25%) to afford the title compound as a colorless oil. MS (ESI) m/z [M+H]⁺625.0.

### Step 3: Synthesis of Compound 4

To a solution of compound 3 (700 mg, 1.21 mmol) in EtOAc (1 mL), NH₃ (7 M in methanol, 1.7 mL) was added. The mixture was stirred vigorously in sealed tube for 12 h at 100° C., then concentrated under reduced pressure. The residue was purified by flash column chromatography (EA/MeOH=10/1) affording the title compound as a white solid (250 mg, 70%). ¹H NMR (400 MHz, MeOD) § 8.48 (s, 1H), 8.16 (s, 1H), 6.09 (s, 1H), 5.42-5.01 (m, 3H), 4.59 (d, J=9.0 Hz, 1H), 4.14-3.80 (m, 3H). MS (ESI) m/z [M+H]⁺294.0.

### Step 4 and 5: Synthesis of Example 31C

The title compound was obtained with similar procedures to those described for Example 8C(9.7 mg, 3.3% for two steps). ¹H NMR (400 MHz, D₂O) δ 8.71 (s, 1H), 8.33 (s, 1H), 6.14 (s, 1H), 5.66-5.51 (m, 2H), 5.43-5.14 (m, 4H), 5.08-5.03 (m, 2H), 4.60-4.58 (m, 1H), 4.47-4.40 (m, 2H), 4.37-4.21 (m, 3H), 4.13-4.01 (m, 1H), 2.18 (s, 3H), 2.09 (s, 3H), 2.00 (s, 3H), 1.96-1.91 (m, 6H). ³¹P NMR (162 MHz, D₂O) δ 43.85, −15.16. MS (ESI) m/z [M−H]⁻870.1.

### Example 31D

### Synthesis of Example 31D

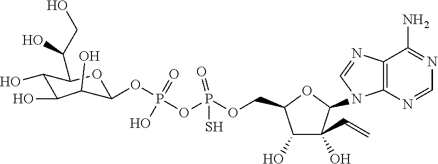 [SMILES: C=C[C@@]1(O)[C@H](O)[C@@H](COP(=O)(S)OP(=O)(O)OC2OC([C@@H](O)CO)C(O)C(O)C2O)O[C@H]1n1cnc2c(N)ncnc21]

The title compound was obtained from Example 31C with similar procedures to those described for Example 1D (9 mg, 59%). ¹H NMR (400 MHz, D₂O) δ 8.77-8.61 (m, 1H), 8.32 (s, 1H), 6.19-6.07 (m, 1H), 5.41-5.16 (m, 3H), 5.04-5.01 (m, 1H), 4.63-4.60 (m, 1H), 4.50-4.42 (m, 1H), 4.38-4.31 (m, 2H), 4.10-4.02 (m, 1H), 3.90-3.86 (m, 1H), 3.78-3.55 (m, 4H), 3.33-3.31 (m, 1H). ³¹P NMR (162 MHz, D₂O) δ 43.54, −14.32. MS (ESI) m/z [M−H]⁻660.1.

### Example 31E

### Synthesis of Example 31E

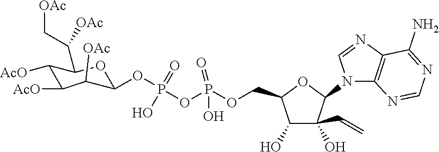 [SMILES: C=C[C@@]1(O)[C@H](O)[C@@H](COP(=O)(O)OP(=O)(O)OC2OC([C@H](COC(C)=O)OC(C)=O)C(OC(C)=O)C(OC(C)=O)C2OC(C)=O)O[C@H]1n1cnc2c(N)ncnc21]

The title compound was obtained with similar procedures to those described for Example 31C(90 mg, 19.7%). ¹H NMR (400 MHz, D₂O) δ 8.57 (s, 1H), 8.29 (s, 1H), 6.16-6.09 (m, 1H), 5.50-5.43 (m, 2H), 5.34-5.23 (m, 2H), 5.20-5.10 (m, 2H), 5.06-4.97 (m, 2H), 4.57-4.52 (m, 1H), 4.43-4.19 (m, 5H), 3.99-3.91 (m, 1H), 2.15 (s, 3H), 2.06 (s, 3H), 1.96 (s, 3H), 1.93 (s, 3H), 1.89 (s, 3H). ³¹P NMR (162 MHz, D₂O) δ 11.20, −14.14. MS (ESI) m/z [M−H]⁻854.1.

### Example 31F

### Synthesis of Example 31F

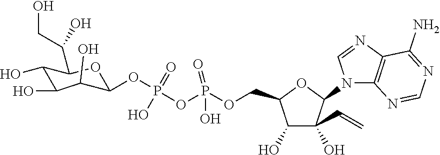 [SMILES: C=C[C@@]1(O)[C@H](O)[C@@H](COP(=O)(O)OP(=O)(O)OC2OC([C@@H](O)CO)C(O)C(O)C2O)O[C@H]1n1cnc2c(N)ncnc21]

The title compound was obtained from Example 31E with similar procedures to those described for Example 1D (2.8 mg, 3.6%). ¹H NMR (400 MHz, D₂O) δ 8.48 (s, 1H), 8.19 (s, 1H), 6.06 (s, 1H), 5.31-5.15 (m, 2H), 5.09 (d, J=8.0 Hz, 1H), 4.94 (dd, J=8.0, 1.6 Hz, 1H), 4.51 (d, J=8.0 Hz, 1H), 4.36-4.30 (m, 1H), 4.25-4.16 (m, 2H), 3.93 (d, J=3.2 Hz, 1H), 3.81-3.76 (m, 1H), 3.69-3.49 (m, 4H), 3.22-3.18 (m, 1H). MS (ESI) m/z [M−H]⁻644.1.

### Example 31K

### Synthesis of Example 31K

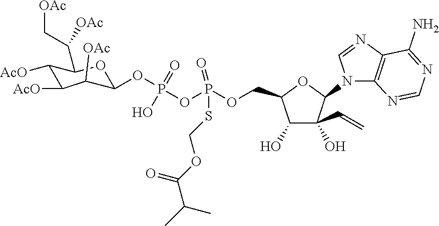 [SMILES: C=C[C@@]1(O)[C@H](O)[C@@H](COP(=O)(OP(=O)(O)OC2OC([C@H](COC(C)=O)OC(C)=O)C(OC(C)=O)C(OC(C)=O)C2OC(C)=O)SCOC(=O)C(C)C)O[C@H]1n1cnc2c(N)ncnc21]

A mixture of Example 31C(70 mg, 80.31 μmol), iodomethyl isobutyrate (36.62 mg, 160.61 μmol) and silver carbonate (26.57 mg, 96.37 μmol) in DMF (1.5 mL) was stirred at rt for 10 min. Filtered to remove the silver salt, the filtrate was purified by prep-HPLC with ACN in water (0.1% FA) 0˜40% to afford the title compound (7.2 mg, 9.2%) as a white solid. ¹H NMR (400 MHz, DMSO-d₆) δ 8.40-8.24 (m, 1H), 8.13-8.12 (m, 1H), 5.98-5.94 (m, 1H), 5.62-5.57 (m, 1H), 5.49-5.39 (m, 2H), 5.37-5.19 (m, 4H), 5.16-5.04 (m, 2H), 4.99-4.90 (m, 1H), 4.51-4.44 (m, 1H), 4.34-4.29 (m, 1H), 4.25-4.20 (m, 1H), 4.16-4.01 (m, 3H), 2.62-2.54 (m, 1H), 2.14-2.11 (m, 3H), 2.02-2.01 (m, 3H), 1.98-1.91 (m, 6H), 1.90-1.88 (m, 3H), 1.12-1.06 (m, 6H). MS (ESI) m/z [M−H]⁻970.1.

### Example 31L

### Synthesis of Example 31L

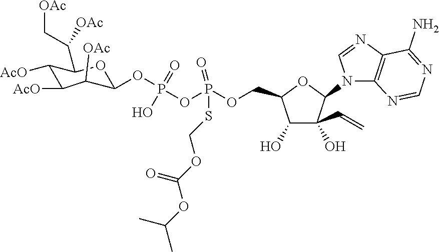 [SMILES: C=C[C@@]1(O)[C@H](O)[C@@H](COP(=O)(OP(=O)(O)OC2OC([C@H](COC(C)=O)OC(C)=O)C(OC(C)=O)C(OC(C)=O)C2OC(C)=O)SCOC(=O)OC(C)C)O[C@H]1n1cnc2c(N)ncnc21]

The title compound was obtained with similar procedures to those described for Example 31K (1.2 mg, 8.2%) using iodomethyl isopropyl carbonate instead of iodomethyl isobutyrate. ¹H NMR (400 MHz, D₂O) δ 8.61-8.53 (m, 1H), 8.23-8.22 (m, 1H), 6.20-6.19 (m, 1H), 5.76-5.62 (m, 3H), 5.60-5.57 (m, 1H), 5.43-5.40 (m, 1H), 5.38-5.35 (m, 1H), 5.25-5.21 (m, 1H), 5.12-5.03 (m, 3H), 4.92-4.86 (m, 1H), 4.67-4.62 (m, 1H), 4.61-4.57 (m, 1H), 4.45-4.38 (m, 2H), 4.35-4.32 (m, 1H), 4.26-4.20 (m, 1H), 3.97-3.87 (m, 1H), 2.24-2.22 (m, 3H), 2.12-2.09 (m, 3H), 2.05 (s, 3H), 2.03-1.98 (m, 6H), 1.33-1.27 (m, 6H). ³¹P NMR (162 MHz, D₂O) δ 45.91-45.67 (m, 1P), −17.84-−18.17 (m, 1P). MS (ESI) m/z [M−H]⁻986.1.

### Example 31H

### Synthesis of Example 31H

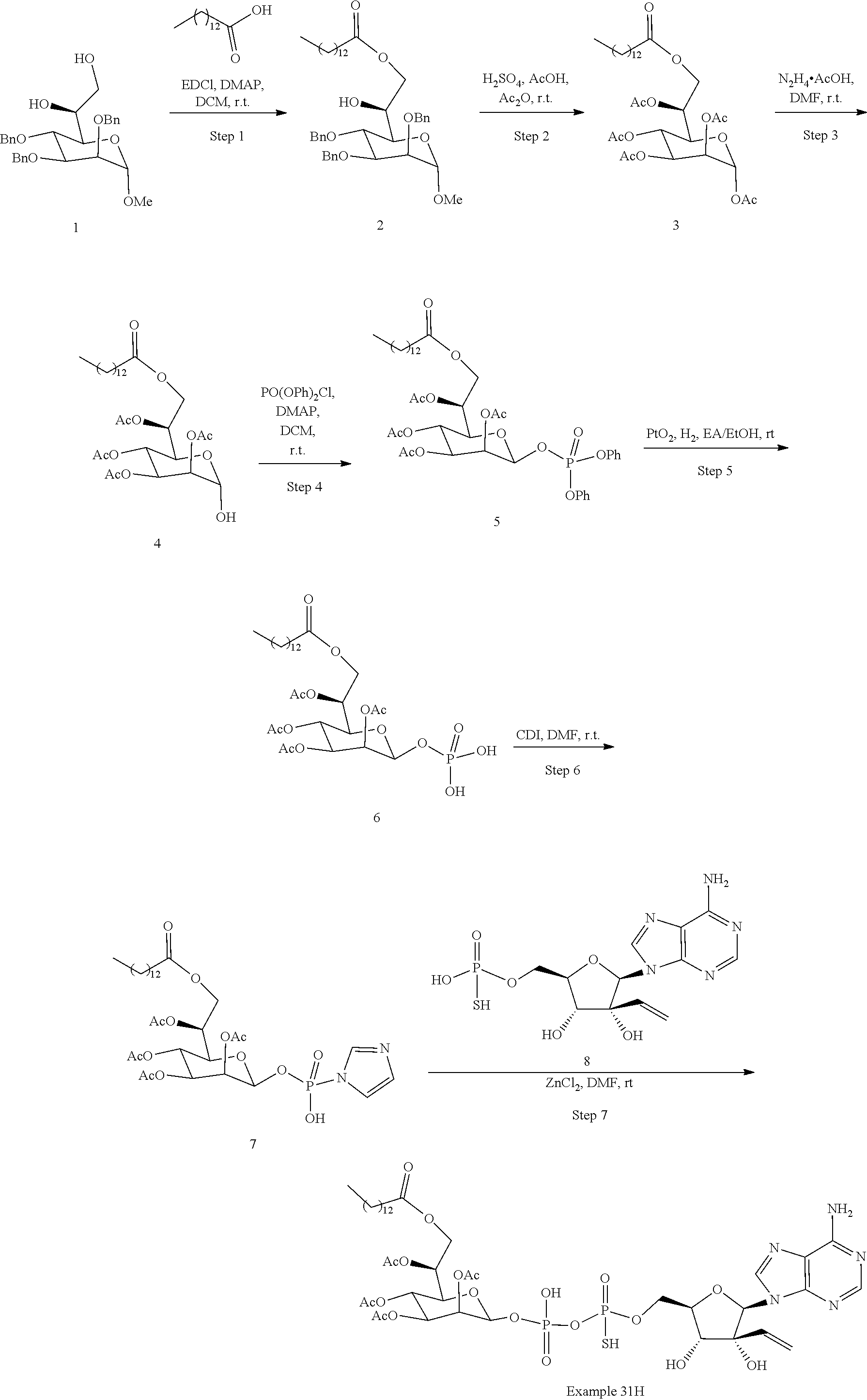

### Step 1: Synthesis of Compound 2

To a solution of compound 1 (1 g, 1.02 mmol) in DCM (10 mL) charged with N₂ was added DMAP (370.52 mg, 3.03 mmol), EDCI (465.13 mg, 2.43 mmol) and myristic acid (554.10 mg, 2.43 mmol), the mixture was stirred at r.t. for 10 h. The reaction was quenched with MeOH (1 mL) and stirred at r.t. for 1 h, then concentrated under reduced pressure, the residue was purified on silica gel chromatography with PE/EA=6/1 to afford the title compound (1.04 g, 72%) as a yellow oil. MS (ESI) m/z [M+Na]⁺727.1.

### Step 2: Synthesis of Compound 3

To a solution of compound 2 (250 mg, 354.65 μmol) in AcOH (1 mL) was added Ac₂O (724.11 mg, 7.09 mmol) and H₂SO₄ (208.70 mg, 2.13 mmol). The mixture was stirred at rt for 24 h, then poured into ice water and extracted with EA, the organic layer was dried over anhydrous Na₂SO₄, filtered and concentrated under reduced pressure. The residue was purified on silica gel chromatography with PE/EA=4/1 to afford the title compound (1.04 g, 72%) as a yellow oil. ¹H NMR (400 MHz, CDCl₃) δ 6.03 (d, J=1.6 Hz, 1H), 5.31-5.13 (m, 4H), 4.22-4.17 (m, 1H), 4.13-4.01 (m, 2H), 2.20 (t, J=7.6 Hz, 2H), 2.13 (s, 3H), 2.10 (s, 3H), 2.06 (s, 3H), 1.96 (s, 3H), 1.93 (s, 3H), 1.56-1.44 (m, 2H), 1.27-1.14 (m, 20H), 0.81 (t, J=6.8 Hz, 3H). MS (ESI) m/z [M+Na]⁺653.1.

### Step 3: Synthesis of Compound 4

To a solution of compound 3 (150 mg, 237.82 μmol) in DMF (3 mL) was added N2H₄·AcOH (32.85 mg, 356.74 μmol), the mixture was stirred at rt for 0.5 h, then the reaction was quenched by adding 10 mL water and extracted with EA, the organic layer was dried over anhydrous Na₂SO₄, concentrated under reduced pressure. The residue was purified on silica gel chromatography with PE/EA=2/1 to afford the title compound (120 mg, 85%) as a colorless oil. ¹H NMR (400 MHz, CDCl₃) δ 5.35-5.29 (m, 1H), 5.27-5.15 (m, 4H), 4.38-4.31 (m, 1H), 4.19-4.14 (m, 1H), 4.13-4.07 (m, 1H), 2.23 (t, J=7.6 Hz, 2H), 2.11 (s, 3H), 2.08 (s, 3H), 1.96 (s, 3H), 1.92 (s, 3H), 1.58-1.44 (m, 2H), 1.26-1.12 (m, 20H), 0.81 (t, J=6.8 Hz, 3H). MS (ESI) m/z [M+Na]⁺611.1.

### Step 4: Synthesis of Compound 5

To a solution of compound 4 (127 mg, 215 μmol) in DCM (10 mL) was added DMAP (52.71 mg, 431.47 μmol) and a solution of diphenyl chlorophosphite (115.91 mg, 431.47 μmol) in DCM (5 mL) slowly. The reaction was stirred at 25° C. for 12 h, then the mixture was dispersed in DCM and water, the organic layer was isolated and concentrated under reduced pressure. The residue was purified with PE/EA=4/1 to afford the title compound (110 mg, β/α-6/1, 62%) as a colorless oil. ¹H NMR (400 MHz, CDCl₃) δ 7.34-7.02 (m, 10H), 5.50 (dd, J=7.0, 1.0 Hz, 1H), 5.45 (d, J=3.1 Hz, 1H), 5.29-5.17 (m, 2H), 4.99 (dd, J=10.1, 3.3 Hz, 1H), 4.27-4.13 (m, 1H), 4.07-4.00 (m, 1H), 3.75 (dd, J=10.0, 2.4 Hz, 1H), 2.18 (t, J=7.6 Hz, 2H), 2.04 (s, 6H), 1.94 (s, 3H), 1.90 (s, 3H), 1.55-1.43 (m, 2H), 1.26-1.13 (m, 20H), 0.80 (t, J=6.8 Hz, 3H). MS (ESI) m/z [M+Na]⁺844.1.

### Step 5: Synthesis of Compound 6

To a solution of compound 5 (150 mg, 179 μmol) in EA (3 mL) and EtOH (3 mL) was added PtO₂ (7.1 mg, 31 μmol). The reaction was stirred at 25° C. under H₂ atmosphere for 24 h, then the mixture was filtered and the filtrate was concentrated under reduced pressure to give the title compound (96 mg, 80%) as a white solid. MS (ESI) m/z [M+H]⁺669.1

### Step 6 and 7: Synthesis of Example 31H

The title compound was obtained with similar procedures to those described for Example 31C. ¹H NMR (400 MHz, MeOD) δ 8.71 (s, 1H), 8.23-8.18 (m, 1H), 6.06 (s, 1H), 5.72-5.59 (m, 2H), 5.44-5.37 (m, 1H), 5.34-5.26 (m, 2H), 5.22-5.16 (m, 2H), 4.97-4.92 (m, 1H), 4.65-4.48 (m, 3H), 4.45-4.31 (m, 2H), 4.28-4.21 (m, 1H), 4.06-3.98 (m, 1H), 2.22 (t, J=7.6 Hz, 2H), 2.17 (s, 3H), 2.05 (s, 3H), 1.96 (s, 3H), 1.91 (s, 3H), 1.56-1.48 (m, 2H), 1.35-1.24 (m, 20H), 0.89 (t, J=6.8 Hz, 3H). MS (ESI) m/z [M−H]⁻1038.0.

### Example 31M

### Synthesis of Example 31M

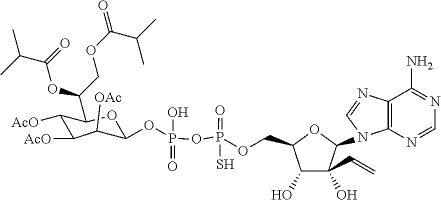 [SMILES: C=C[C@@]1(O)[C@H](O)[C@@H](COP(=O)(S)OP(=O)(O)OC2OC([C@H](COC(=O)C(C)C)OC(=O)C(C)C)C(OC(C)=O)C(OC(C)=O)C2OC(C)=O)O[C@H]1n1cnc2c(N)ncnc21]

The title compound was obtained with similar procedures to those described for Example 31H (60 mg, 39.6% for two steps) by using isobutyric acid instead of myristic acid. ¹H NMR (400 MHz, D₂O) δ 8.64-8.61 (m, 1H), 8.25 (s, 1H), 6.05-6.01 (m, 1H), 5.55-5.45 (m, 2H), 5.32-5.08 (m, 5H), 4.97-4.92 (m, 2H), 4.49 (d, J=9.1 Hz, 1H), 4.41-4.33 (m, 2H), 4.28-4.19 (m, 3H), 4.03-3.97 (m, 1H), 2.57-2.48 (m, 1H), 2.40-2.29 (m, 1H), 2.08-2.05 (m, 1H), 1.92-1.89 (m, 3H), 1.83-1.80 (m, 3H), 1.07-1.04 (m, 3H), 1.01-0.98 (m, 3H), 0.92-0.86 (m, 6H). ³¹P NMR (162 MHz, D₂O) δ 44.26-43.57 (m, 1P), −15.02-−15.26 (m, 1P). MS (ESI) m/z [M−H]⁻926.2.

### Example 310

### Synthesis of Example 310

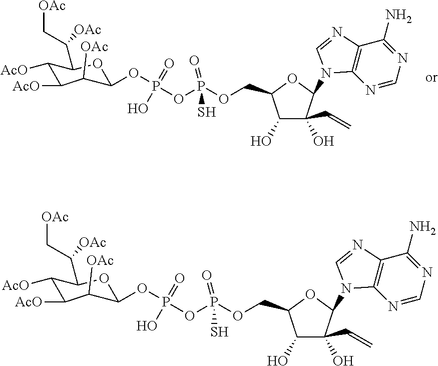 [SMILES: C=C[C@@]1(O)[C@H](O)[C@@H](CO[P@](=O)(S)OP(=O)(O)OC2OC([C@H](COC(C)=O)OC(C)=O)C(OC(C)=O)C(OC(C)=O)C2OC(C)=O)O[C@H]1n1cnc2c(N)ncnc21 ; C=C[C@@]1(O)[C@H](O)[C@@H](CO[P@@](=O)(S)OP(=O)(O)OC2OC([C@H](COC(C)=O)OC(C)=O)C(OC(C)=O)C(OC(C)=O)C2OC(C)=O)O[C@H]1n1cnc2c(N)ncnc21]

The title compound was isolated from Example 31C with prep-HPLC(C18 column, 0.1% FA in water/MeCN=95% to 70%). ¹H NMR (400 MHz, D₂O) δ 8.79 (s, 1H), 8.41 (s, 1H), 6.22 (s, 1H), 5.65 (d, J=8.0 Hz, 1H), 5.63-5.60 (m, 1H), 5.47-5.41 (m, 1H), 5.40-5.31 (m, 1H), 5.29-5.23 (m, 2H), 5.15-5.08 (m, 2H), 4.65 (d, J=8.0 Hz, 1H), 4.52-4.46 (m, 2H), 4.43-4.29 (m, 3H), 4.15-4.10 (m, 1H), 2.24 (s, 3H), 2.14 (s, 3H), 2.05 (s, 3H), 2.00 (s, 3H), 1.98 (s, 3H). MS (ESI) m/z [M−H]⁻870.1.

### Example 31P

### Synthesis of Example 31P

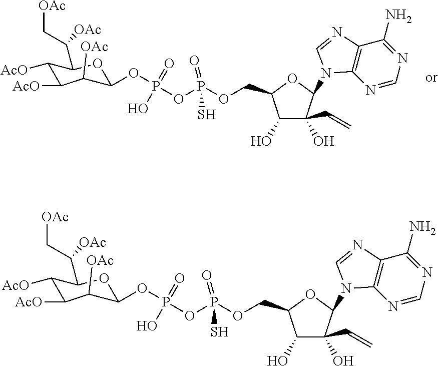 [SMILES: C=C[C@@]1(O)[C@H](O)[C@@H](CO[P@@](=O)(S)OP(=O)(O)OC2OC([C@H](COC(C)=O)OC(C)=O)C(OC(C)=O)C(OC(C)=O)C2OC(C)=O)O[C@H]1n1cnc2c(N)ncnc21 ; C=C[C@@]1(O)[C@H](O)[C@@H](CO[P@](=O)(S)OP(=O)(O)OC2OC([C@H](COC(C)=O)OC(C)=O)C(OC(C)=O)C(OC(C)=O)C2OC(C)=O)O[C@H]1n1cnc2c(N)ncnc21]

The title compound was isolated from Example 31C with prep-HPLC(C18 column, 0.1% FA in water/MeCN=95% to 70%). ¹H NMR (400 MHz, D₂O) δ 8.72 (s, 1H), 8.43 (s, 1H), 6.13 (s, 1H), 5.65 (d, J=8.0 Hz, 1H), 5.63-5.60 (m, 1H), 5.47-5.41 (m, 1H), 5.41-5.32 (m, 1H), 5.31-5.25 (m, 2H), 5.17-5.09 (m, 2H), 4.64 (d, J=8.0 Hz, 1H), 4.51-4.44 (m, 2H), 4.39-4.30 (m, 3H), 4.17-4.13 (m, 1H), 2.24 (s, 3H), 2.15 (s, 3H), 2.07 (s, 3H), 2.01 (s, 3H), 1.98 (s, 3H). MS (ESI) m/z [M−H]⁻870.1.

### Example 31Q

### Synthesis of Example 31Q

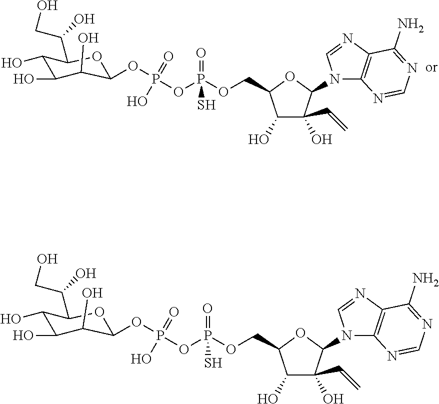 [SMILES: C=C[C@@]1(O)[C@H](O)[C@@H](CO[P@](=O)(S)OP(=O)(O)OC2OC([C@@H](O)CO)C(O)C(O)C2O)O[C@H]1n1cnc2c(N)ncnc21 ; C=C[C@@]1(O)[C@H](O)[C@@H](CO[P@@](=O)(S)OP(=O)(O)OC2OC([C@@H](O)CO)C(O)C(O)C2O)O[C@H]1n1cnc2c(N)ncnc21]

The title compound was isolated from Example 31D with prep-HPLC(C18 column, 0.1% FA in water/MeCN=100% to 95%) as TEA salt (1 equiv.). ¹H NMR (400 MHz, D₂O) δ 8.72 (s, 1H), 8.32 (s, 1H), 6.21 (s, 1H), 5.45-5.39 (m, 1H), 5.37 (dd, J=2.4, 10.4 Hz, 1H), 5.33 (d, J=8.8 Hz, 1H), 5.09 (d, J=10.4 Hz, 1H), 4.69 (d, J=8.0 Hz, 1H), 4.56-4.49 (m, 1H), 4.47-4.37 (m, 2H), 4.16 (d, J=3.2 Hz, 1H), 3.95 (t, J=8.0 Hz, 1H), 3.85-3.66 (m, 4H), 3.39 (d, J=9.8 Hz, 1H), 3.21 (d, J=7.3 Hz, 7H), 1.28 (t, J=7.3 Hz, 11H). ³¹P NMR (162 MHz, D₂O) δ 43.64 (d, J=28 Hz, 1P), −14.21 (d, J=28 Hz, 1P). MS (ESI) m/z [M−H]: 660.1.

### Example 31R

### Synthesis of Example 31R

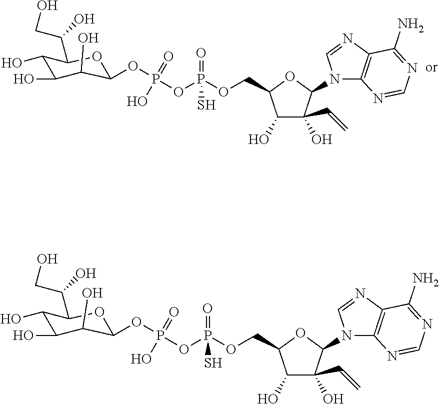 [SMILES: C=C[C@@]1(O)[C@H](O)[C@@H](CO[P@@](=O)(S)OP(=O)(O)OC2OC([C@@H](O)CO)C(O)C(O)C2O)O[C@H]1n1cnc2c(N)ncnc21 ; C=C[C@@]1(O)[C@H](O)[C@@H](CO[P@](=O)(S)OP(=O)(O)OC2OC([C@@H](O)CO)C(O)C(O)C2O)O[C@H]1n1cnc2c(N)ncnc21]

The title compound was isolated from Example 31D with prep-HPLC(C18 column, 0.1% FA in water/MeCN=100% to 95%) as TEA salt (1 equiv.). ¹H NMR (400 MHz, D₂O) δ 8.67 (s, 1H), 8.41 (s, 1H), 6.09 (s, 1H), 5.43-5.36 (m, 1H), 5.34 (d, J=8.2 Hz, 1H), 5.31-5.22 (m, 1H), 5.06 (d, J=10.4 Hz, 1H), 4.61 (d, J=8.0 Hz, 1H), 4.58-4.53 (m, 1H), 4.45-4.36 (m, 2H), 4.16 (d, J=3.2 Hz, 1H), 3.97 (t, J=8.0 Hz, 1H), 3.87-3.69 (m, 4H), 3.42 (d, J=9.6 Hz, 1H), 3.20 (d, J=7.3 Hz, 5H), 1.28 (t, J=7.3 Hz, 8H). ³¹P NMR (162 MHz, D₂O) δ 43.52 (d, J=27 Hz, 1P), −14.29 (d, J=27 Hz, 1P). MS (ESI) m/z [M−H]⁻660.1.

### Example 33C

### Synthesis of Example 33C

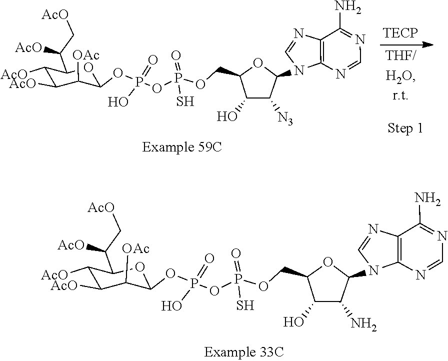 [SMILES: CC(=O)OC[C@H](OC(C)=O)C1OC(OP(=O)(O)OP(=O)(S)OC[C@H]2O[C@@H](n3cnc4c(N)ncnc43)[C@H](N=[N+]=[N-])[C@@H]2O)C(OC(C)=O)C(OC(C)=O)C1OC(C)=O ; CC(=O)OC[C@H](OC(C)=O)C1OC(OP(=O)(O)OP(=O)(S)OC[C@H]2O[C@@H](n3cnc4c(N)ncnc43)[C@H](N)[C@@H]2O)C(OC(C)=O)C(OC(C)=O)C1OC(C)=O]

To a solution of Example 59C(50 mg, 0.057 mmol) in THF/H₂O (1 mL/1 mL) was added TECP (66 mg, 0.23 mmol). The mixture was stirred at room temperature for 2 h. The mixture was purified by prep-HPLC(Daisogel-C18-5-100, 0˜20% acetonitrile in 0.1% FA aqueous solution, retention time: 25-35 min) followed with lyophilization to afford the title compound (42 mg, 87%) as a white solid. ¹H NMR (400 MHz, D₂O) δ 8.70-8.63 (m, 1H), 8.35 (s, 1H), 6.47 (d, J=7.2 Hz, 1H), 5.64-5.54 (m, 2H), 5.28-5.18 (m, 2H), 5.10-5.03 (m, 1H), 4.82-4.80 (m, 1H), 4.60-4.55 (m, 1H), 4.52-4.48 (m, 1H), 4.43-4.36 (m, 1H), 4.32-4.18 (m, 3H), 4.11-4.03 (m, 1H), 2.17 (s, 3H), 2.13-2.08 (m, 3H), 2.04-2.00 (m, 3H), 1.99-1.95 (m, 3H), 1.93 (s, 3H). ³¹P NMR (162 MHz, D₂O) δ 43.85, −15.22. MS (ESI) m/z [M−H]⁻842.8.

### Example 33E

### Synthesis of Example 33E

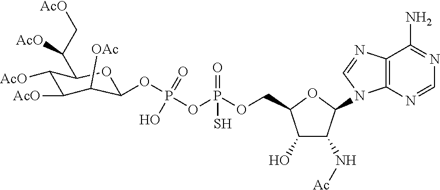 [SMILES: CC(=O)N[C@@H]1[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1cnc2c(N)ncnc21]

A solution of Example 33C(20 mg, 0.23 mmol) in Ac₂O (2 mL) was stirred at 25° C. for 4 h. Concentrated under reduced pressure and the residue was purified by prep-HPLC with ACN in H₂O 0˜25% to give the title compound (1.2 mg, 5.5%) as white solid. ¹H NMR (400 MHz, D₂O) δ 8.66 (s, 1H), 8.33 (s, 1H), 6.10 (d, J=7.5 Hz, 1H), 5.55-5.49 (m, 2H), 5.20-5.14 (m, 2H), 5.05-4.98 (m, 1H), 4.92-4.87 (m, 1H), 4.53-4.49 (m, 1H), 4.41-4.32 (m, 2H), 4.26-4.14 (m, 3H), 4.04-3.98 (m, 1H), 2.13 (s, 3H), 2.05 (s, 3H), 1.96 (s, 3H), 1.92 (s, 3H), 1.88 (s, 3H), 1.83 (s, 3H). MS (ESI) m/z [M−H]⁻843.1.

### Example 34C

### Synthesis of Example 34C

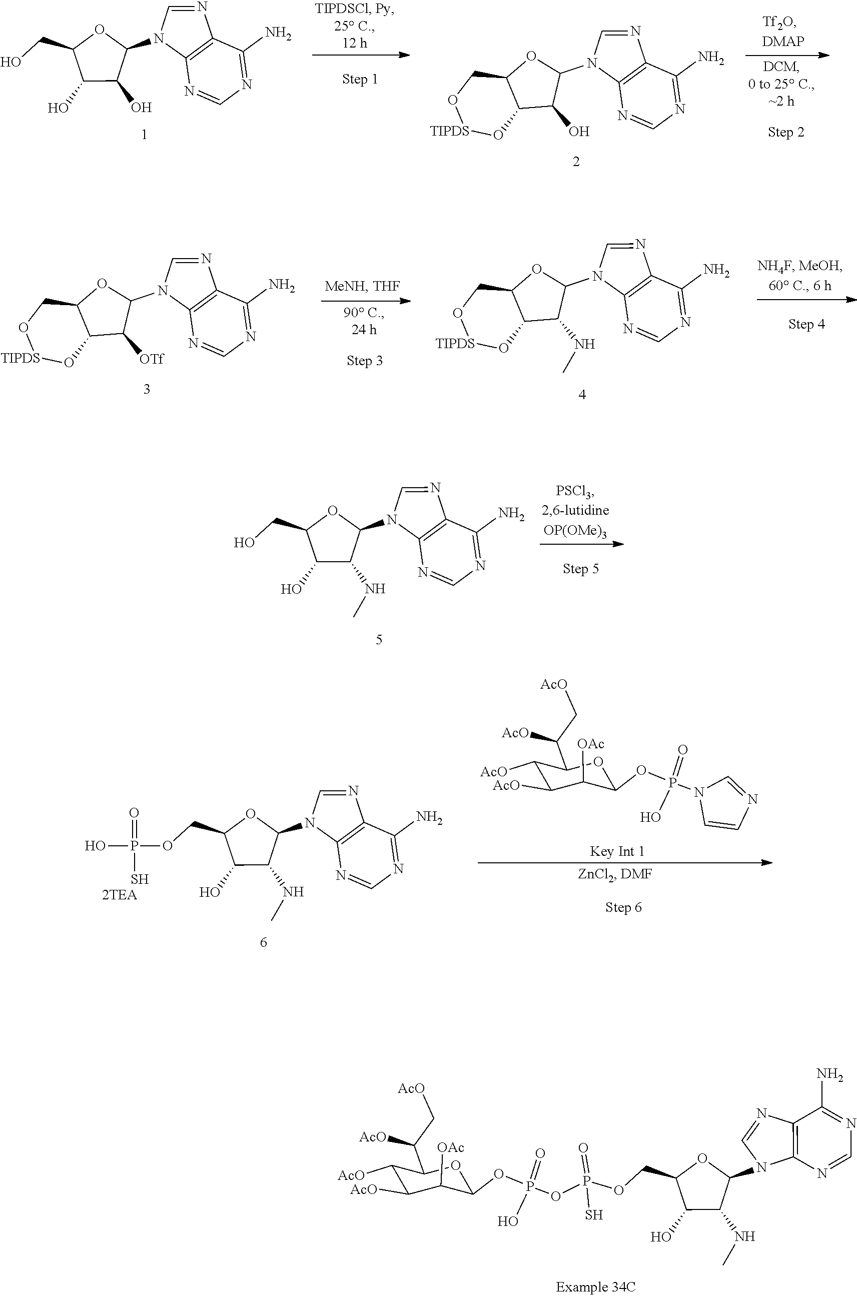

### Step 1: Synthesis of Compound 2

To a solution of compound 1 (44.0 g, 165 mmol) in Pyridine (300 mL) was added TIPDSCI (57.1 g, 181 mmol, 57.9 mL) at 25° C., the reaction mixture was stirred at 25° C. for 12 hrs. The reaction mixture was diluted with Sat. NaHCO₃ (600 mL) and EtOAc (900 mL), the organic phase was separated, washed with water (300 mL×2) and brine (300 mL), dried over Na₂SO₄, filtered and concentrated under reduced pressure to afford the title compound (65.0 g, 72.9%) as a yellow solid. The crude product was used in the next step without further purification. ¹H NMR (400 MHz, CDCl₃) δ 8.59 (d, J=4.0 Hz, 1H), 8.18 (s, 1H), 8.11 (s, 1H), 6.20 (d, J=6.0 Hz, 1H), 6.09 (brs, 2H), 4.62 (d, J=7.6 Hz, 2H), 4.04 (d, J=3.2 Hz, 2H), 3.90-3.80 (m, 1H), 1.20-1.00 (m, 28H).

### Step 2: Synthesis of Compound 3

To a solution of compound 2 (56.5 g, 111 mmol) in DCM (600 mL) was added DMAP (40.6 g, 333 mmol) at −30 to −50° C., after stirred for 30 mins, then Tf₂O (39.1 g, 139 mmol, 22.9 mL) in DCM (100 mL) was added at −30 to −50° C., and the reaction mixture was stirred at 25° C. for 2 h. The reaction mixture was washed with 20% of Citric acid solution (100 mL×5) and organic layer was washed with brine (50 mL), dried over Na₂SO₄, filtered and concentrated under reduced pressure to give the title compound (70.0 g, 69.8%) as a yellow oil. The crude product was used in the next step without further purification. ¹H NMR (400 MHz, CDCl₃) δ 8.33 (s, 1H), 7.93 (s, 1H), 6.40 (d, J=6.0 Hz, 1H), 5.69 (br s, 2H), 5.51-5.46 (m, 1H), 5.41 (d, J=7.2 Hz, 1H), 4.27-4.19 (m, 1H), 4.09 (dd, J=3.3, 12.3 Hz, 1H), 3.97 (br dd, J=3.6, 6.8 Hz, 1H), 1.12-1.04 (m, 28H).

### Step 3: Synthesis of Compound 4

A mixture of compound 3 (3 g, 4.67 mmol) and 2M Methylamine in THF (14 mL) was stirred at 90° C. for 24 h in a sealed tube. Then the reaction was cooled to r.t. and the solvent was removed in vacuo. The crude product was purified by silica gel column chromatography (MeOH/DCM 0˜10%) to give the title compound (1.11 g, 41.9%) as a yellow solid. ¹H NMR (400 MHz, DMSO-d₆) δ 8.22 (s, 1H), 8.04 (s, 1H), 7.29 (s, 2H), 5.85 (d, J=2.2 Hz, 1H), 5.01-4.96 (m, 1H), 3.99-3.95 (m, 2H), 3.92-3.85 (m, 1H), 3.48-3.44 (m, 1H), 2.38 (s, 3H), 1.05-0.99 (m, 28H). MS (ESI) m/z [M+H]⁺522.9.

### Step 4: Synthesis of Compound 5

To a solution of compound 4 (250 mg, 0.48 mmol) in MeOH (4 mL) was added Ammonium fluoride (178 mg, 4.8 mmol) at r.t., the reaction mixture was stirred at 60° C. for 6 h. After evaporated, the crude product was purified by column chromatography (MeOH in EA 0˜30%) to afford the title compound (70 mg, 52.2%) as a white solid. ¹H NMR (400 MHz, DMSO-d₆) δ 8.33 (s, 1H), 8.12 (s, 1H), 7.34 (s, 2H), 5.76 (d, J=7.7 Hz, 1H), 5.47 (s, 1H), 4.29 (dd, J=5.1, 1.9 Hz, 1H), 4.00-3.97 (m, 1H), 3.70-3.63 (m, 2H), 3.59-3.52 (m, 1H), 2.22 (s, 3H). MS (ESI) m/z [M+H]⁺281.1.

### Step 5 and 6: Synthesis of Example 34C

The title compound was obtained with similar procedures to those described for Example 8C(24 mg, 11.2% for two steps). ¹H NMR (400 MHz, D₂O) δ 8.59 (s, 1H), 8.19 (s, 1H), 6.42-6.40 (m, 1H), 5.47-5.42 (m, 2H), 5.12-5.10 (m, 1H), 5.06-5.03 (m, 1H), 4.98-4.92 (m, 1H), 4.79-4.77 (m, 1H), 4.47-4.44 (m, 1H), 4.40 (s, 1H), 4.31-4.27 (m, 1H), 4.18-4.02 (m, 3H), 3.90-3.88 (m, 1H), 2.61 (s, 3H), 2.11-2.07 (m, 3H), 2.04-1.97 (m, 3H), 1.93-1.91 (m, 3H), 1.86-1.83 (m, 6H). ³¹P NMR (162 MHz, D₂O) δ 43.94, −15.25. MS (ESI) m/z [M−H]⁻857.1.

### Example 34D

### Synthesis of Example 34D

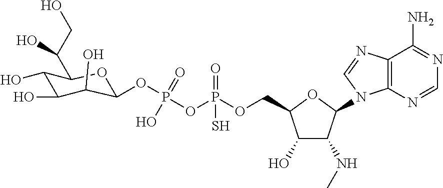 [SMILES: CN[C@@H]1[C@H](O)[C@@H](COP(=O)(S)OP(=O)(O)OC2OC([C@@H](O)CO)C(O)C(O)C2O)O[C@H]1n1cnc2c(N)ncnc21]

Two isomers of the title compound were obtained from Example 34C with similar procedures to those described for Example 1D.

Isomer 1 (3.5 mg, 23.2%): ¹H NMR (400 MHz, D₂O) δ 8.80 (s, 1H), 8.42 (s, 1H), 6.58 (d, J=7.4 Hz, 1H), 5.29 (d, J=8.5 Hz, 1H), 4.96 (d, J=5.3 Hz, 1H), 4.69-4.61 (m, 1H), 4.57 (s, 1H), 4.35-4.18 (m, 2H), 4.11 (d, J=3.2 Hz, 1H), 3.94 (t, J=6.6 Hz, 1H), 3.82-3.63 (m, 4H), 3.38 (d, J=9.7 Hz, 1H), 2.76 (s, 3H). ³¹P NMR (162 MHz, D₂O) δ 43.67, −14.24. MS (ESI) m z [M−H]⁻647.0.

Isomer 2 (1.9 mg, 12.6%): ¹H NMR (400 MHz, D₂O) δ 8.74 (s, 1H), 8.41 (s, 1H), 6.58 (d, J=7.6 Hz, 1H), 5.27 (d, J=8.4 Hz, 1H), 4.96 (dd, J=5.3, 1.4 Hz, 1H), 4.67 (dd, J=7.6, 5.3 Hz, 1H), 4.57 (s, 1H), 4.35-4.20 (m, 2H), 4.11 (d, J=3.2 Hz, 1H), 3.94 (td, J=6.5, 1.6 Hz, 1H), 3.83-3.65 (m, 4H), 3.38 (dd, J=9.7, 1.8 Hz, 1H), 2.76 (s, 3H). ³¹P NMR (162 MHz, D₂O) δ 43.52, −14.31. MS (ESI) m/z [M−H]⁻647.1.

### Example 35C

### Synthesis of Example 35C

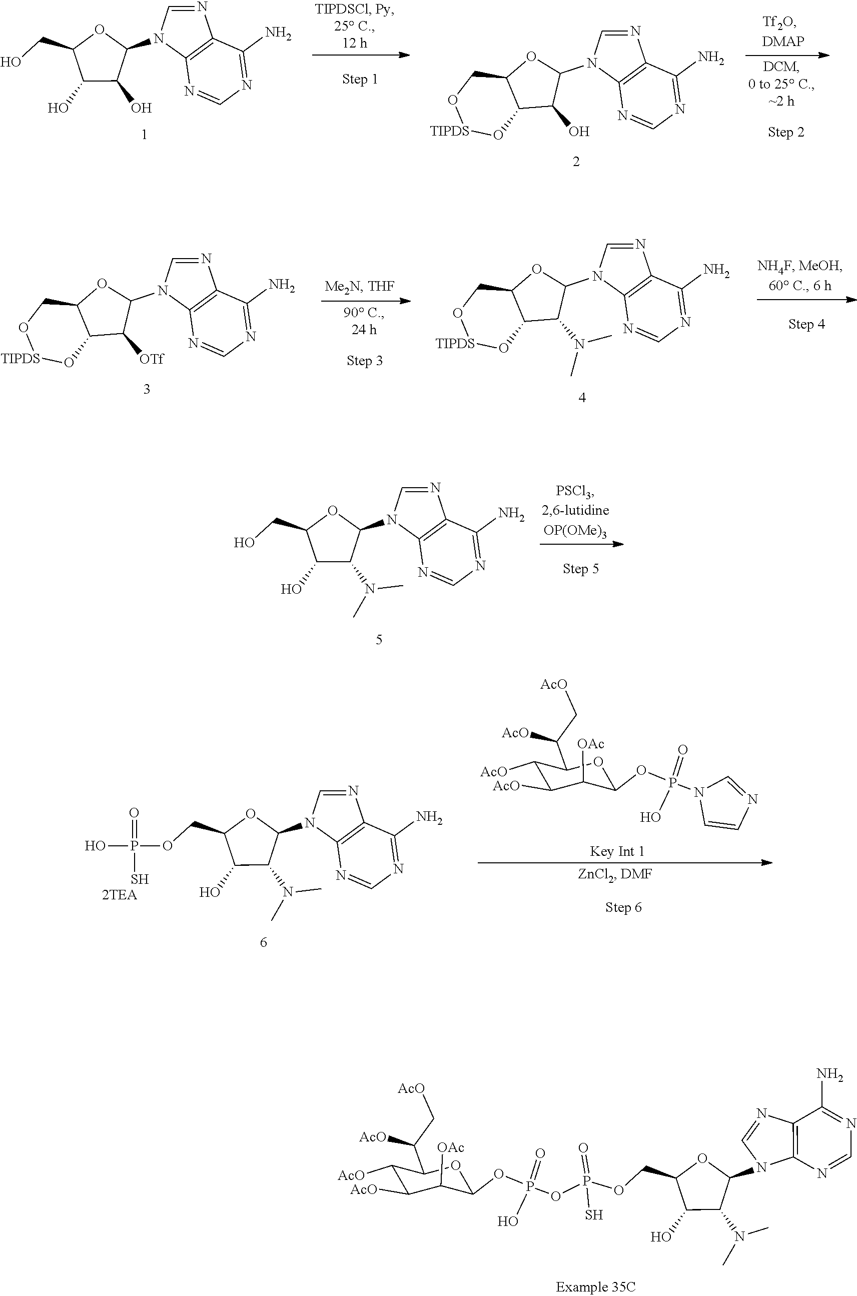

### Step 1: Synthesis of Compound 2

To a solution of compound 1 (44.0 g, 165 mmol) in Pyridine (300 mL) was added TIPDSCI (57.1 g, 181 mmol, 57.9 mL) at 25° C., the reaction mixture was stirred at 25° C. for 12 hrs. The reaction mixture was diluted with Sat. NaHCO₃ (600 mL) and EtOAc (900 mL), the organic phase was separated, washed with water (300 mL×2) and brine (300 mL), dried over Na₂SO₄, filtered and concentrated under reduced pressure to afford the title compound (65.0 g, 72.9%) as a yellow solid. The crude product was used in the next step without further purification. ¹H NMR (400 MHz, CDCl₃) δ 8.59 (d, J=4.0 Hz, 1H), 8.18 (s, 1H), 8.11 (s, 1H), 6.20 (d, J=6.0 Hz, 1H), 6.09 (brs, 2H), 4.62 (d, J=7.6 Hz, 2H), 4.04 (d, J=3.2 Hz, 2H), 3.90-3.80 (m, 1H), 1.20-1.00 (m, 28H).

### Step 2: Synthesis of Compound 3

To a solution of compound 2 (56.5 g, 111 mmol) in DCM (600 mL) was added DMAP (40.6 g, 333 mmol) at −30 to −50° C., after stirred for 30 mins, then Tf₂O (39.1 g, 139 mmol, 22.9 mL) in DCM (100 mL) was added at −30 to −50° C., and the reaction mixture was stirred at 25° C. for 2 h. The reaction mixture was washed with 20% of Citric acid solution (100 mL×5) and organic layer was washed with brine (50 mL), dried over Na₂SO₄, filtered and concentrated under reduced pressure to give the title compound (70.0 g, 69.8%) as a yellow oil. The crude product was used in the next step without further purification. ¹H NMR (400 MHz, CDCl₃) δ 8.33 (s, 1H), 7.93 (s, 1H), 6.40 (d, J=6.0 Hz, 1H), 5.69 (br s, 2H), 5.51-5.46 (m, 1H), 5.41 (d, J=7.2 Hz, 1H), 4.27-4.19 (m, 1H), 4.09 (dd, J=3.3, 12.3 Hz, 1H), 3.97 (br dd, J=3.6, 6.8 Hz, 1H), 1.12-1.04 (m, 28H).

### Step 3: Synthesis of Compound 4

Compound 3 (1.4 g, 2.2 mmol) was added to 2M dimethylamine in THF (14 mL). The resulting solution was stirred at 90° C. for 24 h in a sealed tube. Then the solvent was removed in vacuo. The crude product was purified by silica gel column chromatography, using MeOH/DCM (0˜10%) as eluent to give the title compound (1 g, 85.4%) as a yellow solid. ¹H NMR (400 MHz, DMSO-d₆) δ 8.31 (s, 1H), 8.08 (s, 1H), 7.32 (s, 2H), 6.15 (d, J=3.6 Hz, 1H), 5.19 (t, J=7.2 Hz, 1H), 4.09-4.00 (m, 1H), 3.91 (d, J=5.2 Hz, 2H), 3.88-3.84 (m, 1H), 2.51 (s, 6H), 1.10-1.01 (m, 28H). MS (ESI) m/z [M+H]⁺537.0.

### Step 4: Synthesis of Compound 5

To a solution of Compound 4 (1 g, 1.9 mmol) in MeOH (10 ml) was added NH₄F (700 mg, 1.9 mmol). The resulting solution was stirred at 60° C. for 6 h. Then the solvent was removed in vacuo. The crude product was purified by silica gel column chromatography, using MeOH/DCM (0˜20%) as eluent to give the title compound (321.3 mg, 57.9%) as a yellow solid. ¹H NMR (400 MHz, MeOD) δ 8.44 (s, 1H), 8.20 (s, 1H), 6.31 (d, J=8.4 Hz, 1H), 4.47 (d, J=4.8 Hz, 1H), 4.15 (t, J=2.9 Hz, 1H), 3.84-3.73 (m, 2H), 3.66 (dd, J=8.4, 4.8 Hz, 1H), 2.22 (s, 6H). MS (ESI) m/z [M+H]⁺295.0.

### Step 5 and 6: Synthesis of Example 35C

The title compound was obtained with similar procedures to those described for Example 8C(30 mg, 16.8% for two steps). ¹H NMR (400 MHz, D₂O) δ 8.82 (s, 1H), 8.39 (s, 1H), 6.65 (d, J=8.2 Hz, 1H), 5.65-5.57 (m, 2H), 5.31-5.17 (m, 2H), 5.08 (t, J=10.0 Hz, 1H), 4.94 (d, J=4.9 Hz, 1H), 4.83 (dd, J=8.1, 4.9 Hz, 1H), 4.53-4.49 (m, 1H), 4.44-4.38 (m, 1H), 4.33-4.20 (m, 2H), 4.16-4.09 (m, 2H), 2.84 (brs, 6H), 2.17 (s, 3H), 2.11 (s, 3H), 2.02 (s, 3H), 1.98 (s, 3H), 1.93 (s, 3H) ³¹P NMR (162 MHz, D₂O) δ 43.66, −14.98. MS (ESI) m/z [M−H]⁻871.1.

### Example 35D

### Synthesis of Example 35D

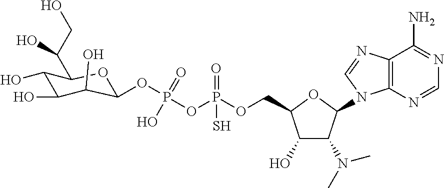 [SMILES: CN(C)[C@@H]1[C@H](O)[C@@H](COP(=O)(S)OP(=O)(O)OC2OC([C@@H](O)CO)C(O)C(O)C2O)O[C@H]1n1cnc2c(N)ncnc21]

The title compound was obtained from Example 35C with similar procedures to those described for Example 1D (7.6 mg, 19.8%). ¹H NMR (400 MHz, D₂O) δ 8.68 (s, 1H), 8.22 (s, 1H), 6.54 (d, J=8.4 Hz, 1H), 5.20 (d, J=8.4 Hz, 1H), 4.92 (d, J=4.6 Hz, 1H), 4.77-4.73 (m, 1H), 4.47 (s, 1H), 4.28-4.17 (m, 1H), 4.15-3.98 (m, 2H), 3.86 (t, J=6.3 Hz, 1H), 3.76-3.58 (m, 4H), 3.31 (d, J=9.7 Hz, 1H), 2.77 (brs, 6H). ³¹P NMR (162 MHz, D₂O) δ 43.53, −14.23. MS (ESI) m/z [M−H]⁻661.2.

### Example 35E

### Synthesis of Example 35E

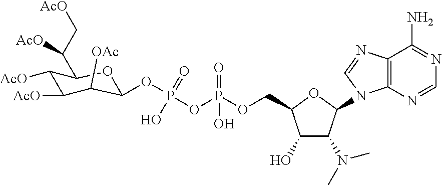 [SMILES: CC(=O)OC[C@H](OC(C)=O)C1OC(OP(=O)(O)OP(=O)(O)OC[C@H]2O[C@@H](n3cnc4c(N)ncnc43)[C@H](N(C)C)[C@@H]2O)C(OC(C)=O)C(OC(C)=O)C1OC(C)=O]

The title compound was obtained from the preparation of Example 35C(340 mg, 64.6%). ¹H NMR (400 MHz, D₂O) δ 8.64 (s, 1H), 8.32 (s, 1H), 6.57 (d, J=8.2 Hz, 1H), 5.45-5.40 (m, 2H), 5.20-5.15 (m, 1H), 5.14-5.09 (m, 1H), 5.02-4.94 (m, 1H), 4.83 (d, J=4.9 Hz, 1H), 4.74-4.68 (m, 1H), 4.43-4.39 (m, 1H), 4.32-4.26 (m, 1H), 4.22-4.15 (m, 1H), 4.14-4.08 (m, 1H), 4.07-3.98 (m, 2H), 2.74 (brs, 6H), 2.07 (s, 3H), 2.01 (s, 3H), 1.92 (s, 3H), 1.89 (s, 3H), 1.83 (s, 3H) ³¹P NMR (162 MHz, D₂O) δ-11.52 (d, 1P), −14.10 (d, 1P). MS (ESI) m/z [M−H]⁻855.1.

### Example 35F

### Synthesis of Example 35F

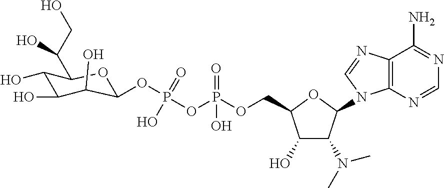 [SMILES: CN(C)[C@@H]1[C@H](O)[C@@H](COP(=O)(O)OP(=O)(O)OC2OC([C@@H](O)CO)C(O)C(O)C2O)O[C@H]1n1cnc2c(N)ncnc21]

The title compound was obtained from Example 35E with similar procedures to those described for Example 1D (49 mg, 64.9%). ¹H NMR (400 MHz, D₂O) δ 8.58 (s, 1H), 8.23 (s, 1H), 6.52 (d, J=8.2 Hz, 1H), 5.11 (d, J=8.2 Hz, 1H), 4.85 (d, J=5.0 Hz, 1H), 4.74-4.67 (m, 1H), 4.18-4.01 (m, 2H), 3.94 (d, J=3.2 Hz, 1H), 3.86-3.78 (m, 1H), 3.72-3.64 (m, 1H), 3.64-3.52 (m, 3H), 3.30-3.22 (m, 1H), 2.73 (brs, 6H). ³¹P NMR (162 MHz, D₂O) δ-11.56 (d, 1P), −13.16. MS (ESI) m/z [M−H]⁻645.1.

### Example 35G

### Synthesis of Example 35G

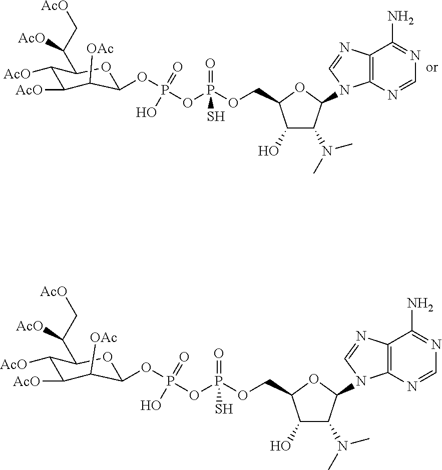 [SMILES: CC(=O)OC[C@H](OC(C)=O)C1OC(OP(=O)(O)O[P@@](=O)(S)OC[C@H]2O[C@@H](n3cnc4c(N)ncnc43)[C@H](N(C)C)[C@@H]2O)C(OC(C)=O)C(OC(C)=O)C1OC(C)=O ; CC(=O)OC[C@H](OC(C)=O)C1OC(OP(=O)(O)O[P@](=O)(S)OC[C@H]2O[C@@H](n3cnc4c(N)ncnc43)[C@H](N(C)C)[C@@H]2O)C(OC(C)=O)C(OC(C)=O)C1OC(C)=O]

The title compound was isolated from Example 35C with prep-HPLC(C18 column, 0.1% FA in water/MeCN=95% to 70%). ¹H NMR (400 MHz, D₂O) δ 8.84 (s, 1H), 8.32 (s, 1H), 6.58 (d, J=8.2 Hz, 1H), 5.53-5.46 (m, 2H), 5.18-5.10 (m, 2H), 5.01-4.94 (m, 1H), 4.87 (d, J=5.2 Hz, 1H), 4.72-4.67 (m, 1H), 4.44-4.41 (m, 1H), 4.33-4.27 (m, 1H), 4.21-4.12 (m, 2H), 4.06-3.98 (m, 2H), 2.74 (brs, 6H), 2.08 (s, 3H), 2.00 (s, 3H), 1.92 (s, 3H), 1.87 (s, 3H), 1.84 (s, 3H). ³¹P NMR (162 MHz, D₂O) δ 43.94, −15.09. MS (ESI) m/z [M−H]⁻871.1.

### Example 35H

### Synthesis of Example 35H

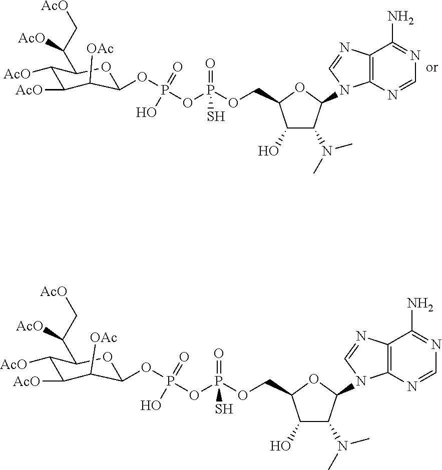 [SMILES: CC(=O)OC[C@H](OC(C)=O)C1OC(OP(=O)(O)O[P@](=O)(S)OC[C@H]2O[C@@H](n3cnc4c(N)ncnc43)[C@H](N(C)C)[C@@H]2O)C(OC(C)=O)C(OC(C)=O)C1OC(C)=O ; CC(=O)OC[C@H](OC(C)=O)C1OC(OP(=O)(O)O[P@@](=O)(S)OC[C@H]2O[C@@H](n3cnc4c(N)ncnc43)[C@H](N(C)C)[C@@H]2O)C(OC(C)=O)C(OC(C)=O)C1OC(C)=O]

The title compound was isolated from Example 35C with prep-HPLC(C18 column, 0.1% FA in water/MeCN=95% to 70%). ¹H NMR (400 MHz, D₂O) δ 8.74 (s, 1H), 8.33 (s, 1H), 6.57 (d, J=8.2 Hz, 1H), 5.56-5.49 (m, 2H), 5.20-5.11 (m, 2H), 5.03-4.96 (m, 1H), 4.86 (d, J=5.2 Hz, 1H), 4.77-4.72 (m, 1H), 4.44-4.40 (m, 1H), 4.35-4.29 (m, 1H), 4.24-4.11 (m, 2H), 4.08-4.00 (m, 2H), 2.75 (brs, 6H), 2.08 (s, 3H), 2.02 (s, 3H), 1.93 (s, 3H), 1.89 (s, 3H), 1.84 (s, 3H). ³¹P NMR (162 MHz, D₂O) δ 43.50, −15.11. MS (ESI) m/z [M−H]⁻871.1.

### Example 35I

### Synthesis of Example 35I

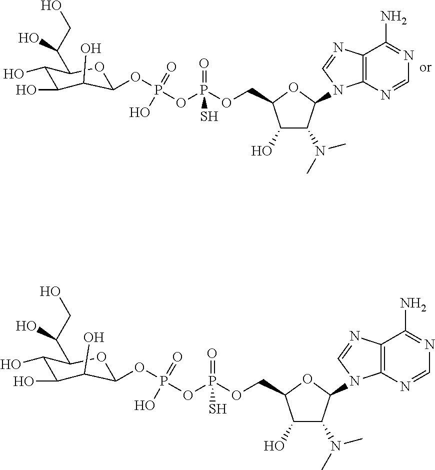 [SMILES: CN(C)[C@@H]1[C@H](O)[C@@H](CO[P@](=O)(S)OP(=O)(O)OC2OC([C@@H](O)CO)C(O)C(O)C2O)O[C@H]1n1cnc2c(N)ncnc21 ; CN(C)[C@@H]1[C@H](O)[C@@H](CO[P@@](=O)(S)OP(=O)(O)OC2OC([C@@H](O)CO)C(O)C(O)C2O)O[C@H]1n1cnc2c(N)ncnc21]

The title compound was isolated from Example 35D with prep-HPLC(C18 column, 0.1% FA in water/MeCN=98% to 90%). ¹H NMR (400 MHz, D₂O) δ 8.81 (s, 1H), 8.31 (s, 1H), 6.56 (d, J=8.4 Hz, 1H), 5.18 (d, J=8.6 Hz, 1H), 4.88 (d, J=5.0 Hz, 1H), 4.76-4.71 (m, 1H), 4.47-4.42 (m, 1H), 4.23-4.15 (m, 1H), 4.10-4.03 (m, 1H), 3.99 (d, J=3.1 Hz, 1H), 3.85-3.77 (m, 1H), 3.72-3.52 (m, 4H), 3.26 (dd, J=9.7, 1.6 Hz, 1H), 2.74 (brs, 6H). ³¹P NMR (162 MHz, D₂O) δ 43.79 (d, 1P), −14.17 (d, 1P). MS (ESI) m/z [M−H]⁻661.1.

### Example 35J

### Synthesis of Example 35J

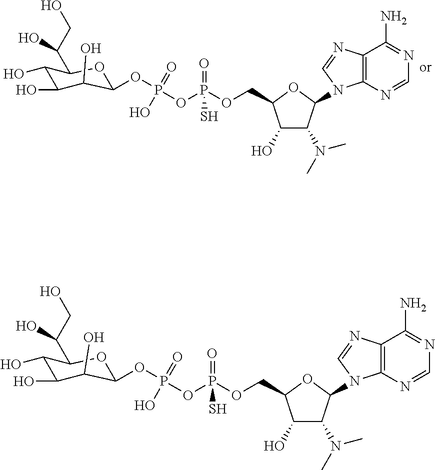 [SMILES: CN(C)[C@@H]1[C@H](O)[C@@H](CO[P@@](=O)(S)OP(=O)(O)OC2OC([C@@H](O)CO)C(O)C(O)C2O)O[C@H]1n1cnc2c(N)ncnc21 ; CN(C)[C@@H]1[C@H](O)[C@@H](CO[P@](=O)(S)OP(=O)(O)OC2OC([C@@H](O)CO)C(O)C(O)C2O)O[C@H]1n1cnc2c(N)ncnc21]

The title compound was isolated from Example 35D with prep-HPLC(C18 column, 0.1% FA in water/MeCN=98% to 90%). ¹H NMR (400 MHz, D₂O) δ 8.72 (s, 1H), 8.29 (s, 1H), 6.56 (d, J=8.2 Hz, 1H), 5.16 (d, J=8.4 Hz, 1H), 4.89 (d, J=5.0 Hz, 1H), 4.79-4.74 (m, 1H), 4.45-4.41 (m, 1H), 4.22-4.15 (m, 1H), 4.09-4.03 (m, 1H), 3.99 (d, J=3.1 Hz, 1H), 3.86-3.77 (m, 1H), 3.71-3.54 (m, 4H), 3.26 (dd, J=9.7, 1.6 Hz, 1H), 2.76 (brs, 6H). ³¹P NMR (162 MHz, D₂O) δ 43.56 (d, 1P), −14.20 (d, 1P). MS (ESI) m/z [M−H]⁻661.1.

### Example 35K

### Synthesis of Example 35K

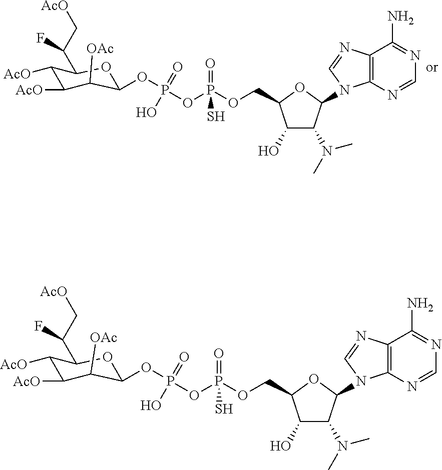 [SMILES: CC(=O)OC[C@H](F)C1OC(OP(=O)(O)O[P@@](=O)(S)OC[C@H]2O[C@@H](n3cnc4c(N)ncnc43)[C@H](N(C)C)[C@@H]2O)C(OC(C)=O)C(OC(C)=O)C1OC(C)=O ; CC(=O)OC[C@H](F)C1OC(OP(=O)(O)O[P@](=O)(S)OC[C@H]2O[C@@H](n3cnc4c(N)ncnc43)[C@H](N(C)C)[C@@H]2O)C(OC(C)=O)C(OC(C)=O)C1OC(C)=O]

The title compound was prepared from key Int 2 with similar procedures to those described for Example 35C and was isolated with prep-HPLC(C18 column, 0.1% FA in water/MeCN=95% to 70%). ¹H NMR (400 MHz, D₂O) δ 8.97 (s, 1H), 8.47 (s, 1H), 6.73 (d, J=8.2 Hz, 1H), 5.69 (d, J=8.0 Hz, 1H), 5.64 (d, J=2.8 Hz, 1H), 5.41-5.29 (m, 2H), 5.03 (d, J=5.2 Hz, 1H), 4.90-4.85 (m, 1H), 4.60-4.58 (m, 1H), 4.51-4.47 (m, 1H), 4.45-4.41 (m, 1H), 4.38-4.31 (m, 1H), 4.23-4.16 (m, 1H), 4.14-4.03 (m, 1H), 2.94 (brs, 6H), 2.24 (s, 3H), 2.14 (s, 3H), 2.10 (s, 3H), 2.03 (s, 3H). ³¹P NMR (162 MHz, D₂O) δ 43.89 (d, 1P), −15.26 (d, 1P). MS (ESI) m/z [M−H]⁻831.1.

### Example 35L

### Synthesis of Example 35L

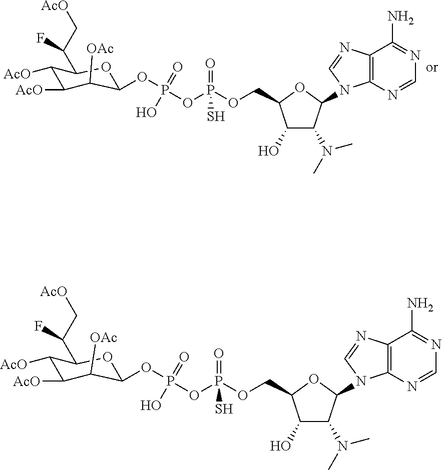 [SMILES: CC(=O)OC[C@H](F)C1OC(OP(=O)(O)O[P@](=O)(S)OC[C@H]2O[C@@H](n3cnc4c(N)ncnc43)[C@H](N(C)C)[C@@H]2O)C(OC(C)=O)C(OC(C)=O)C1OC(C)=O ; CC(=O)OC[C@H](F)C1OC(OP(=O)(O)O[P@@](=O)(S)OC[C@H]2O[C@@H](n3cnc4c(N)ncnc43)[C@H](N(C)C)[C@@H]2O)C(OC(C)=O)C(OC(C)=O)C1OC(C)=O]

The title compound was prepared from key Int 2 with similar procedures to those described for Example 35C and was isolated with prep-HPLC(C18 column, 0.1% FA in water/MeCN=95% to 70%). ¹H NMR (400 MHz, D₂O) δ 8.71 (s, 1H), 8.32-8.28 (m, 1H), 6.56 (d, J=8.2 Hz, 1H), 5.56-5.50 (m, 2H), 5.26-5.12 (m, 2H), 4.85 (d, J=4.8 Hz, 1H), 4.77-4.73 (m, 1H), 4.70-4.65 (m, 1H), 4.43-4.39 (m, 1H), 4.37-4.22 (m, 2H), 4.18-4.10 (m, 1H), 4.07-3.99 (m, 1H), 3.99-3.87 (m, 1H), 2.74 (brs, 6H), 2.07 (s, 3H), 1.97 (s, 3H), 1.95 (s, 3H), 1.86 (s, 3H). ³¹P NMR (162 MHz, D₂O) δ 43.69 (d, 1P), −15.19 (d, 1P). MS (ESI) m/z [M−H]⁺831.1.

### Example 36C

### Synthesis of Example 36C

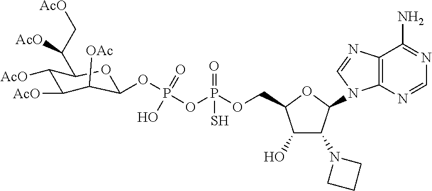 [SMILES: CC(=O)OC[C@H](OC(C)=O)C1OC(OP(=O)(O)OP(=O)(S)OC[C@H]2O[C@@H](n3cnc4c(N)ncnc43)[C@H](N3CCC3)[C@@H]2O)C(OC(C)=O)C(OC(C)=O)C1OC(C)=O]

The title compound was obtained with similar procedures to those described for Example 34C(1.5 mg, 0.4%) by using azetidine instead of methylamine. MS (ESI) m/z [M−H]⁻883.1.

### Example 37C

### Synthesis of Example 37C

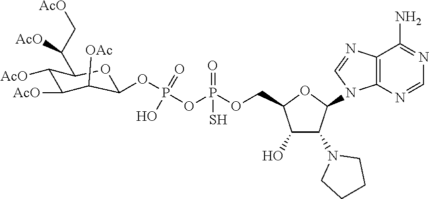 [SMILES: CC(=O)OC[C@H](OC(C)=O)C1OC(OP(=O)(O)OP(=O)(S)OC[C@H]2O[C@@H](n3cnc4c(N)ncnc43)[C@H](N3CCCC3)[C@@H]2O)C(OC(C)=O)C(OC(C)=O)C1OC(C)=O]

The title compound was obtained with similar procedures to those described for Example 34C(50 mg, 34.8%) by using pyrrolidine instead of methylamine. ¹H NMR (400 MHz, D₂O) δ 8.74-8.75 (m, 1H), 8.34 (s, 1H), 6.56 (d, J=8.0 Hz, 1H), 5.59-5.52 (m, 2H), 5.26-5.12 (m, 2H), 5.05-4.99 (m, 1H), 4.88-4.83 (m, 1H), 4.67-4.62 (m, 1H), 4.46 (s, 1H), 4.40-4.31 (m, 1H), 4.28-4.17 (m, 2H), 4.15-4.03 (m, 2H), 3.83 (brs, 1H), 3.33 (brs, 1H), 3.11 (brs, 1H), 2.55 (brs, 1H), 2.12 (s, 3H), 2.06-2.05 (m, 3H), 2.01-1.72 (m, 13H). ³¹P NMR (162 MHz, D₂O) δ 43.79 (dd, J=28.1, 10.2 Hz, 1P), −15.24 (dd, J=28.0, 14.3 Hz, 1P). MS (ESI) m/z [M−H]⁻897.1.

### Example 37D

### Synthesis of Example 37D

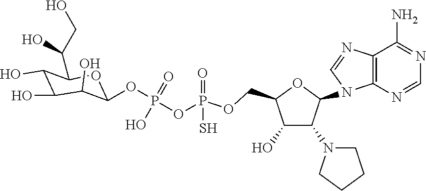 [SMILES: Nc1ncnc2c1ncn2[C@@H]1O[C@H](COP(=O)(S)OP(=O)(O)OC2OC([C@@H](O)CO)C(O)C(O)C2O)[C@@H](O)[C@H]1N1CCCC1]

The title compound was obtained from Example 37C with similar procedures to those described for Example 1D (6 mg, 31.3%). ¹H NMR (400 MHz, D₂O) δ 8.79-8.67 (m, 1H), 8.28 (s, 1H), 6.53-6.48 (m, 1H), 5.19-5.13 (m, 1H), 4.86-4.80 (m, 1H), 4.70-4.65 (m, 1H), 4.45-4.41 (m, 1H), 4.24-4.14 (m, 1H), 4.13-4.04 (m, 1H), 3.99-3.96 (m, 1H), 3.86-3.73 (m, 2H), 3.71-3.53 (m, 4H), 3.39-3.19 (m, 2H), 3.12-2.96 (m, 1H), 2.47 (brs, 1H), 2.00-1.65 (m, 4H). ³¹P NMR (162 MHz, D₂O) δ 43.68, −14.20. MS (ESI) m/z [M−H]⁻687.1.

### Example 39E

### Synthesis of Example 39E

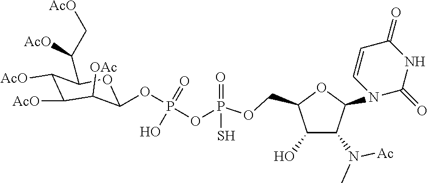 [SMILES: CC(=O)OC[C@H](OC(C)=O)C1OC(OP(=O)(O)OP(=O)(S)OC[C@H]2O[C@@H](n3ccc(=O)[nH]c3=O)[C@H](N(C)C(C)=O)[C@@H]2O)C(OC(C)=O)C(OC(C)=O)C1OC(C)=O]

The title compound was obtained with similar procedures to those described for Example 40C(4 mg, 3.3%) by using N-methylacetamide instead of methylamine. ¹H NMR (400 MHz, D₂O) δ 8.04-7.93 (m, 1H), 6.40-6.33 (m, 1H), 5.96-5.91 (m, 1H), 5.64-5.46 (m, 2H), 5.34-5.17 (m, 2H), 5.08-5.02 (m, 1H), 4.77 (t, J=7.0 Hz, 1H), 4.49-4.37 (m, 2H), 4.31-4.25 (m, 2H), 4.25-4.12 (m, 2H), 4.11-4.05 (m, 1H), 3.12-2.97 (m, 3H), 2.16 (s, 3H), 2.11-2.07 (m, 6H), 2.02-1.95 (m, 6H), 1.93-1.89 (m, 3H). ³¹P NMR (162 MHz, D₂O) δ 44.34-43.76 (m, 1P), −15.11-−15.42 (m, 1P). MS (ESI) m/z [M−H]⁻986.1.

### Example 40C

### Synthesis of Example 40C

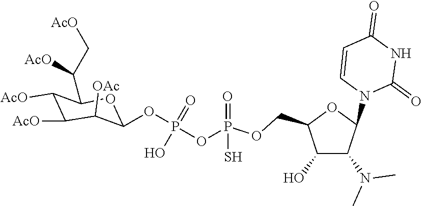 [SMILES: CC(=O)OC[C@H](OC(C)=O)C1OC(OP(=O)(O)OP(=O)(S)OC[C@H]2O[C@@H](n3ccc(=O)[nH]c3=O)[C@H](N(C)C)[C@@H]2O)C(OC(C)=O)C(OC(C)=O)C1OC(C)=O]

The title compound was obtained with similar procedures to those described for Example 34C(21 mg, 13%) by using dimethylamine instead of methylamine and using 1-((2R,3S,4S,5R)-3,4-dihydroxy-5-(hydroxymethyl)tetrahydrofuran-2-yl)pyrimidine-2,4 (1H,3H)-dione as the starting material. ¹H NMR (400 MHz, D₂O) δ 8.01 (dd, J=28.4, 8.4 Hz, 1H), 6.48 (d, J=8.4 Hz, 1H), 6.03 (d, J=8.0 Hz, 1H), 5.65-5.47 (m, 2H), 5.26-5.20 (m, 2H), 5.00-5.00 (m, 1H), 4.78 (t, J=4.0 Hz, 1H), 4.40-4.00 (m, 7H), 2.99 (s, 3H), 2.76 (s, 3H), 2.14 (s, 3H), 2.08-2.03 (m, 3H), 1.99-1.96 (m, 3H), 1.95-1.92 (m, 3H), 1.90 (s, 3H). ³¹P NMR (162 MHz, D₂O) δ 43.95-43.61, −15.15-−15.52. MS (ESI) m/z [M−H]⁻848.0.

### Example 40D

### Synthesis of Example 40D

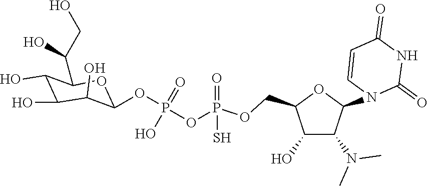 [SMILES: CN(C)[C@@H]1[C@H](O)[C@@H](COP(=O)(S)OP(=O)(O)OC2OC([C@@H](O)CO)C(O)C(O)C2O)O[C@H]1n1ccc(=O)[nH]c1=O]

The title compound was obtained from Example 40C with similar procedures to those described for Example 1D (2.8 mg, 47%). ¹H NMR (400 MHz, D₂O) δ 8.01 (dd, J=26.8, 8.0 Hz, 1H), 6.44 (d, J=8.4 Hz, 1H), 5.93 (d, J=8.0 Hz, 1H), 5.16 (t, J=8.0 Hz, 1H), 4.75 (d, J=4.2 Hz, 1H), 4.30 (d, J=2.0 Hz, 1H), 4.26-4.09 (m, 2H), 4.09-3.96 (m, 2H), 3.85-3.79 (m, 1H), 3.71-3.52 (m, 4H), 3.29-3.23 (m, 1H), 2.92 (s, 3H), 2.74 (s, 3H). ³¹P NMR (162 MHz, D₂O) δ 43.63, −14.27. MS (ESI) m/z [M−H]⁻638.1.

### Example 41C

### Synthesis of Example 41C

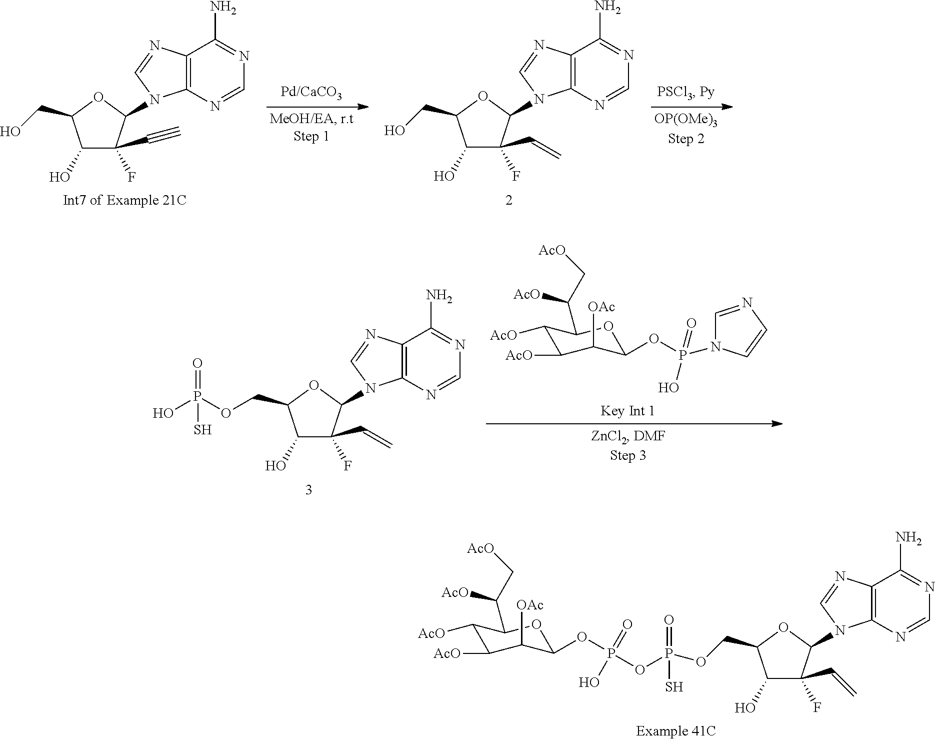 [SMILES: C#C[C@@]1(F)[C@H](O)[C@@H](CO)O[C@H]1n1cnc2c(N)ncnc21 ; C=C[C@@]1(F)[C@H](O)[C@@H](CO)O[C@H]1n1cnc2c(N)ncnc21 ; C=C[C@@]1(F)[C@H](O)[C@@H](COP(=O)(O)S)O[C@H]1n1cnc2c(N)ncnc21 ; CC(=O)OC[C@H](OC(C)=O)C1OC(OP(=O)(O)n2ccnc2)C(OC(C)=O)C(OC(C)=O)C1OC(C)=O ; C=C[C@@]1(F)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1cnc2c(N)ncnc21]

### Step 1: Synthesis of Compound 2

Lindlar catalyst (400.78 mg, 954.81 μmol) was added to a solution of compound 7 of Example 21C(280 mg, 954.81 μmol) in Ethyl Acetate (10 mL) and Methanol (10 mL) at r.t., the reaction mixture was stirred at r.t under H₂ for 5 min, filtered, the solvent was removed under reduced pressure to afford the title compound (220 mg, 78%) as a white solid. ¹H NMR (400 MHz, DMSO-d₆) δ 8.39 (s, 1H), 8.13 (s, 1H), 7.35 (s, 2H), 6.24-6.19 (m, 1H), 5.73 (d, J=7.7 Hz, 1H), 5.42-5.36 (m, 2H), 5.28 (t, J=5.1 Hz, 1H), 5.18-5.14 (m, 1H), 4.71-4.61 (m, 1H), 4.01-3.99 (m, 1H), 3.90-3.84 (m, 1H), 3.79-3.69 (m, 1H). ¹⁹F NMR (376 MHz, DMSO-d₆) δ-169.87. MS (ESI) m/z [M+H]⁺296.1.

### Step 2 and 3: Synthesis of Example 41C

The title compound was obtained with similar procedures to those described for Example 8C(90 mg, 13.7% for two steps). ¹H NMR (400 MHz, D₂O) δ 8.66-8.65 (m, 1H), 8.36 (s, 1H), 6.41-6.37 (m, 1H), 5.63-5.55 (m, 2H), 5.51-5.46 (m, 1H), 5.44-5.36 (m, 1H), 5.28-5.16 (m, 3H), 5.10-5.04 (m, 1H), 4.51-4.47 (m, 1H), 4.45-4.27 (m, 5H), 4.11-4.07 (m, 1H), 2.18 (s, 3H), 2.09 (s, 3H), 2.01 (s, 3H), 1.97-1.96 (m, 3H), 1.92 (s, 3H). ³¹P NMR (162 MHz, D₂O) δ 44.16-43.59 (m),43.69; −14.19-−15.20 (m). ¹⁹F NMR (376 MHz, D₂O) δ-171.86, −171.92. MS (ESI) m/z [M−H]⁻872.1.

### Example 41D

### Synthesis of Example 41D

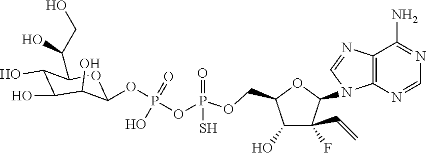 [SMILES: C=C[C@@]1(F)[C@H](O)[C@@H](COP(=O)(S)OP(=O)(O)OC2OC([C@@H](O)CO)C(O)C(O)C2O)O[C@H]1n1cnc2c(N)ncnc21]

Two isomers of the title compound were obtained from Example 41C with similar procedures to those described for Example 1D.

Isomer 1 (5.1 mg, 16.8%): ¹H NMR (400 MHz, D₂O) δ 8.64 (s, 1H), 8.36 (s, 1H), 6.44-6.35 (m, 1H), 5.52-5.36 (m, 2H), 5.27-5.17 (m, 2H), 4.54-4.44 (m, 1H), 4.43-4.32 (m, 2H), 4.09 (d, J=2.8 Hz, 1H), 3.90-3.87 (m, 1H), 3.82-3.71 (m, 2H), 3.71-3.57 (m, 3H), 3.33 (d, J=8.7 Hz, 1H). ³¹P NMR (162 MHz, D₂O) δ 43.92, −14.26. MS (ESI) m/z [M−H]⁻662.1.

Isomer 2 (3.3 mg, 10.9%): ¹H NMR (400 MHz, D₂O) δ 8.66-8.65 (m, 1H), 8.35 (s, 1H), 6.45-6.38 (m, 1H), 5.53-5.35 (m, 2H), 5.28-5.15 (m, 2H), 4.82-4.73 (m, 1H), 4.54-4.45 (m, 1H), 4.38-4.33 (m, 2H), 4.07 (d, J=3.1 Hz, 1H), 3.90-3.87 (m, 1H), 3.82-3.56 (m, 4H), 3.33-3.30 (m, 1H). ³¹P NMR (162 MHz, D₂O) δ 43.54 (d, J=27.6 Hz, 1P), −14.26 (d, J=27.6 Hz, 1P). MS (ESI) m/z [M−H]⁻662.1.

### Example 42C

### Synthesis of Example 42C

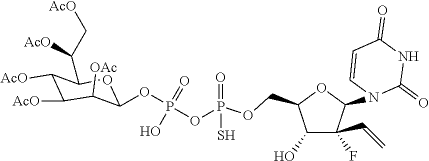 [SMILES: C=C[C@@]1(F)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1ccc(=O)[nH]c1=O]

The title compound was obtained with similar procedures to those described for Example 41C(76.5 mg, 71.9%) by using compound 6 in the preparation of Example 22C as the starting material. ¹H NMR (400 MHz, D₂O) δ 7.91-7.88 (m, 1H), 6.09-6.05 (m, 1H), 5.86-5.83 (m, 1H), 5.70-5.58 (m, 1H), 5.51-5.38 (m, 3H), 5.36-5.31 (m, 1H), 5.18-5.12 (m, 2H), 4.98 (t, J=10.0 Hz, 1H), 4.38-4.25 (m, 3H), 4.22-4.12 (m, 3H), 4.01-3.96 (m, 1H), 2.09 (s, 3H), 2.02 (s, 3H), 1.93 (s, 3H), 1.91 (s, 3H), 1.86 (s, 3H). ³¹P NMR (162 MHz, D₂O) δ 44.04-43.61,-15.17-−15.34. MS (ESI) m/z [M−H]⁻849.1.

### Example 42D

### Synthesis of Example 42D

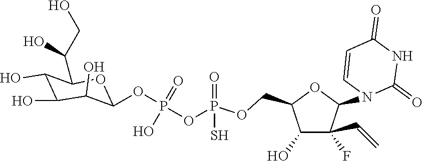 [SMILES: C=C[C@@]1(F)[C@H](O)[C@@H](COP(=O)(S)OP(=O)(O)OC2OC([C@@H](O)CO)C(O)C(O)C2O)O[C@H]1n1ccc(=O)[nH]c1=O]

The title compound was obtained from Example 42C with similar procedures to those described for Example 1D (2 equiv. of TEA salt, 3 mg, 8.7%). ¹H NMR (400 MHz, D₂O) δ 7.96-7.94 (m, 1H), 6.16-6.11 (m, 1H), 5.90-5.88 (m, 1H), 5.75-5.63 (m, 1H), 5.49-5.36 (m, 2H), 5.21 (d, J=8.0 Hz, 1H), 4.45-4.36 (m, 2H), 4.27-4.20 (m, 2H), 4.04 (t, J=2.4 Hz, 1H), 3.86 (t, J=6.4 Hz, 1H), 3.75-3.55 (m, 4H), 3.30 (d, J=9.6 Hz, 1H), 3.10 (q, J=7.2 Hz, 8H), 1.17 (t, J=7.2 Hz, 12H). ³¹P NMR (162 MHz, D₂O) δ 43.67, −14.36. MS (ESI) m/z [M−H]⁻639.0.

### Example 43C

### Synthesis of Example 43C

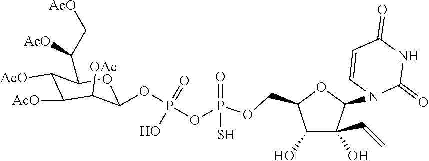 [SMILES: C=C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1ccc(=O)[nH]c1=O]

The title compound was obtained with similar procedures to those described for Example 31C(38 mg, 15.2%) by using compound 6 in the preparation of Example 22C as the starting material. ¹H NMR (400 MHz, D₂O) δ 8.05 (dd, J=7.9, 3.6 Hz, 1H), 5.96-5.89 (m, 2H), 5.66 -5.52 (m, 2H), 5.44-5.36 (m, 1H), 5.34-5.16 (m, 3H), 5.07 (t, J=9.9 Hz, 1H), 4.51-4.36 (m, 3H), 4.31-4.16 (m, 4H), 4.13-4.04 (m, 1H), 2.18 (s, 3H), 2.09 (s, 3H), 2.02-1.97 (m, 6H), 1.93 (s, 3H). ³¹P NMR (162 MHz, D₂O) δ 43.72, −15.28. MS (ESI) m/z [M−H]⁻847.1.

### Example 43D

### Synthesis of Example 43D

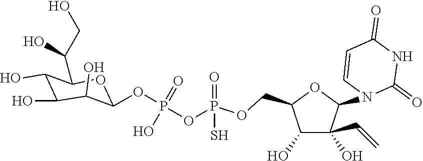 [SMILES: C=C[C@@]1(O)[C@H](O)[C@@H](COP(=O)(S)OP(=O)(O)OC2OC([C@@H](O)CO)C(O)C(O)C2O)O[C@H]1n1ccc(=O)[nH]c1=O]

The title compound was obtained from Example 43C with similar procedures to those described for Example 1D (1.5 equiv. of TEA salt, 4.4 mg, 29.6%). ¹H NMR (400 MHz, D₂O) δ 8.07-7.89 (m, 1H), 5.93-5.84 (m, 2H), 5.66-5.56 (m, 1H), 5.40-5.32 (m, 1H), 5.28-5.22 (m, 2H), 4.44-4.34 (m, 1H), 4.33-4.08 (m, 3H), 4.06-4.01 (m, 1H), 3.89-3.82 (m, 1H), 3.75-3.57 (m, 4H), 3.30 (d, J=9.6 Hz, 1H), 3.10 (q, J=7.3 Hz, 9H), 1.18 (t, J=7.3 Hz, 14H). ³¹P NMR (162 MHz, D₂O) δ 43.55-43.25 (m), −14.22-−14.45 (m). MS (ESI) m/z [M−H]⁻637.1.

### Example 44C

### Synthesis of Example 44C

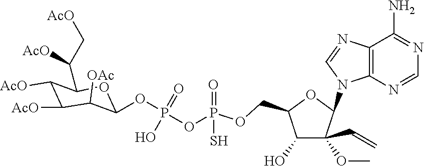 [SMILES: C=C[C@@]1(OC)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1cnc2c(N)ncnc21]

The title compound was obtained with similar procedures to those described for Example 45C(35.8 mg, 31%) by using 6-chloro-9H-purine in stead of 1H-pyrimidine-2,4-dione. ¹H NMR (400 MHz, D₂O) δ 8.76-8.60 (m, 1H), 8.51-8.42 (m, 1H), 6.17-6.07 (m, 1H), 5.64-5.52 (m, 2H), 5.39-5.19 (m, 5H), 5.10-5.01 (m, 1H), 4.67-4.61 (m, 1H), 4.44-4.25 (m, 5H), 4.13-4.06 (m, 1H), 3.46-3.26 (m, 3H), 2.16-2.13 (m, 3H), 2.07 (s, 3H), 1.98 (s, 3H), 1.96-1.92 (m, 3H), 1.89 (s, 3H). ³¹P NMR (162 MHz, D₂O) δ 43.87, −14.32. MS (ESI) m/z [M−H]⁻884.1.

### Example 45C

### Synthesis of Example 45C

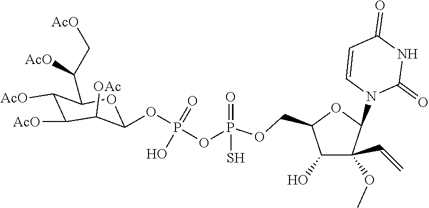 [SMILES: C=C[C@@]1(OC)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1ccc(=O)[nH]c1=O]

The title compound was obtained from Compound 11 of Example 95C with similar procedures to those described for Example 41C(39.2 mg, 38.4%). ¹H NMR (400 MHz, D₂O) δ 7.97-7.91 (m, 1H), 6.07 (s, 1H), 5.85-5.79 (m, 1H), 5.69-5.60 (m, 1H), 5.52-5.44 (m, 2H), 5.35-5.28 (m, 2H), 5.18-5.11 (m, 2H), 5.02-4.94 (m, 1H), 4.38-4.27 (m, 2H), 4.25-4.07 (m, 4H), 4.01-3.95 (m, 1H), 3.40 (s, 3H), 2.11-2.07 (m, 3H), 2.02-1.98 (m, 3H), 1.92 (s, 3H), 1.91-1.87 (m, 3H), 1.85 (s, 3H). ³¹P NMR (162 MHz, D₂O) δ 43.75, −15.29. MS (ESI) m/z [M−H]⁻861.1.

### Example 45E

### Synthesis of Example 45E

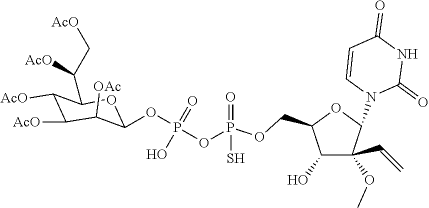 [SMILES: C=C[C@@]1(OC)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@@H]1n1ccc(=O)[nH]c1=O]

The title compound was isolated from the preparation of Example 45C(20 mg, 36%). ¹H NMR (400 MHz, D₂O) δ 7.85-7.77 (m, 1H), 6.25-6.10 (m, 1H), 6.02-5.91 (m, 1H), 5.85-5.78 (m, 1H), 5.66-5.58 (m, 1H), 5.58-5.43 (m, 3H), 5.29-5.13 (m, 2H), 5.08-5.01 (m, 1H), 4.44-4.38 (m, 2H), 4.36-4.14 (m, 3H), 4.11-3.94 (m, 2H), 3.23-3.13 (m, 3H), 2.19-2.13 (m, 3H), 2.10-2.06 (m, 3H), 2.00-1.95 (m, 6H), 1.93-1.89 (m, 3H). ³¹P NMR (162 MHz, D₂O) δ 44.00, −15.21. MS (ESI) m/z [M−H]⁻861.1.

### Example 94C

### Synthesis of Example 94C

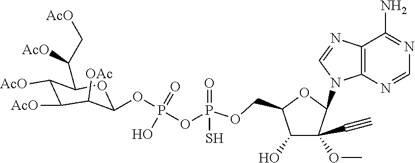 [SMILES: C#C[C@@]1(OC)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1cnc2c(N)ncnc21]

The title compound was obtained with similar procedures to those described for Example 95C(48.7 mg, 23.4%) by using 6-chloro-9H-purine instead of 1H-pyrimidine-2,4-dione. ¹H NMR (400 MHz, D₂O) δ 8.73 (s, 1H), 8.47 (s, 1H), 6.16 (s, 1H), 5.66-5.49 (m, 2H), 5.26-5.17 (m, 2H), 5.09-5.00 (m, 1H), 4.58-4.50 (m, 1H), 4.44-4.22 (m, 5H), 4.11-4.03 (m, 1H), 3.43 (s, 3H), 3.08-3.01 (m, 1H), 2.19-2.12 (m, 3H), 2.06 (s, 3H), 1.99-1.92 (m, 6H), 1.89 (s, 3H). ³¹P NMR (162 MHz, D₂O) δ 44.01, −15.24. MS (ESI) m/z [M−H]⁻882.1.

### Example 95C

### Synthesis of Example 95C

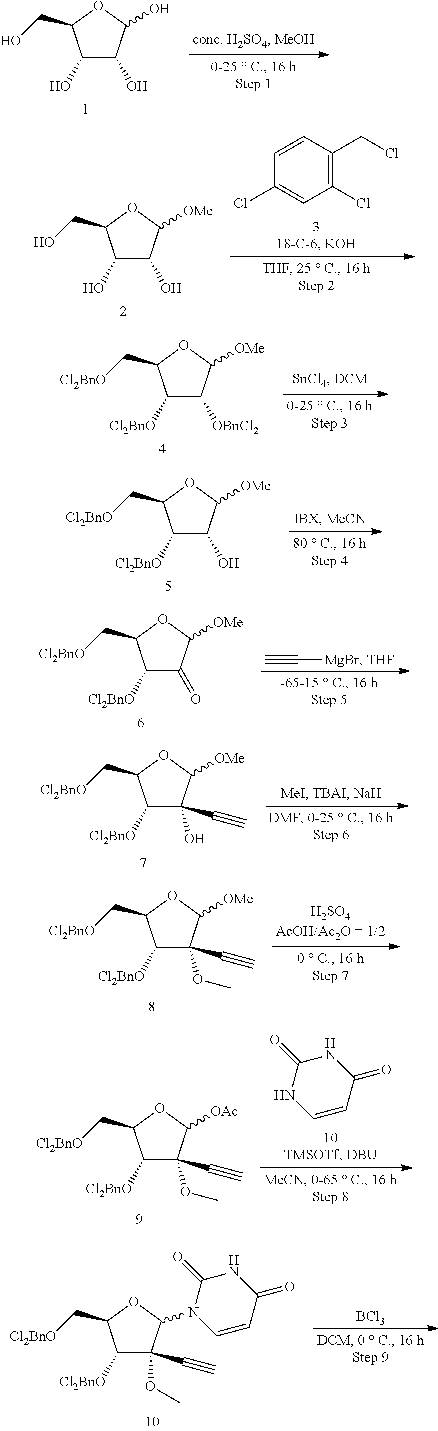

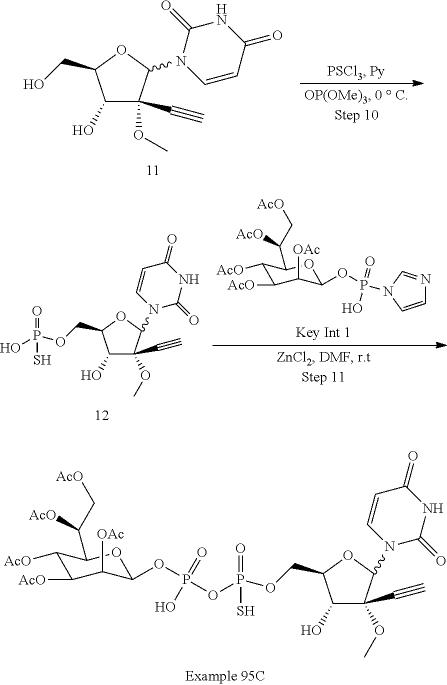 [SMILES: C#C[C@]1(OC)C(n2ccc(=O)[nH]c2=O)O[C@H](CO)[C@H]1O ; "PSCl3, Py" ; C#C[C@]1(OC)C(n2ccc(=O)[nH]c2=O)O[C@H](COP(=O)(O)S)[C@H]1O ; CC(=O)OC[C@H](OC(C)=O)C1OC(OP(=O)(O)n2ccnc2)C(OC(C)=O)C(OC(C)=O)C1OC(C)=O ; C#C[C@]1(OC)C(n2ccc(=O)[nH]c2=O)O[C@H](COP(=O)(S)OP(=O)(O)OC2OC([C@H](COC(C)=O)OC(C)=O)C(OC(C)=O)C(OC(C)=O)C2OC(C)=O)[C@H]1O]

### Step 1: Synthesis of Compound 2

To a solution of compound 1 (210 g, 1.40 mol) in MeOH (1.50 L) was added conc. H₂SO₄ (13.7 g, 140 mmol, 7.46 mL) at 0° C. The reaction mixture was stirred at 25° C. for 16 h. Solid NaHCO₃ was added to adjust pH=7˜8. The mixture was filtered and the filtrate was concentrated under reduced pressure to give the title compound (459 g, crude) as a yellow oil. The crude product was used to next step without further purification. ¹H NMR (400 MHz CDCl₃) δ 5.01-4.91 (m, 1H), 4.16-4.01 (m, 3H), 3.80-3.62 (m, 2H), 3.44-3.41 (m, 3H).

### Step 2: Synthesis of Compound 4

To a solution of compound 2 (50.0 g, 305 mmol) and 18-C-6 (6.44 g, 24.4 mmol) in THF (500 mL) was added KOH (256 g, 4.57 mol) at 25° C. The reaction mixture was stirred at 25° C. for 1 h, then was added to a solution of 2,4-dichloro-1-(chloromethyl)benzene (268 g, 1.37 mol, 190 mL) in THF at 25° C. The resulting mixture was stirred at 25° C. for 15 h. Filtered and the filtrate was concentrated under reduced pressure to give a residue. The residue was purified by column chromatography (EA in PE=20˜50%) to give the title compound (112 g, 57.1%) as a yellow oil. ¹H NMR (400 MHz CDCl₃) δ 7.43-7.34 (m, 6H), 7.23-7.17 (m, 3H), 5.00 (s, 1H), 4.76-4.70 (m, 2H), 4.64-4.62 (m, 4H), 4.43-4.33 (m, 1H), 4.20-4.17 (m, 1H), 3.99-3.98 (m, 1H), 3.74-3.67 (m, 2H), 3.38 (s, 3H).

### Step 3: Synthesis of Compound 5

To a solution of compound 4 (112 g, 174 mmol) in DCM (560 mL) was added SnCl₄ (46.2 g, 177 mmol, 20.7 mL) at 0° C. The mixture was stirred at 25° C. for 16 h. The mixture was neutralized with aqueous NaHCO₃ (1.50 L) to pH=7˜8, filtered, the filtrate was washed with brine (1.50 L). The organic layer was separated and dried to give a residue which was purified by column chromatography (EA in PE=2˜100%) to give the title compound (58.0 g, 69.1%) as white solid. ¹H NMR (400 MHz CDCl₃) δ 7.47 (d, J=8.4 Hz, 1H), 7.40-7.32 (m, 3H), 7.27-7.17 (m, 2H), 5.02 (s, 1H), 4.91 (d, J=12.8 Hz, 1H), 4.70 (d, J=8.4 Hz, 1H), 4.65-4.59 (m, 2H), 4.33-4.29 (m, 1H), 4.04-4.02 (m, 1H), 3.72 (d, J=5.2 Hz, 2H), 3.58 (s, 3H), 3.50 (s, 3H), 2.63 (s, 1H).

### Step 4: Synthesis of Compound 6

To a solution of compound 5 (58.0 g, 120 mmol) in CH₃CN (410 mL) was added IBX (50.5 g, 180 mmol) at 25° C. The mixture was stirred at 80° C. for 16 h, cooled to r.t. and was filtered, the filtrate was concentrated under reduced pressure. The residue was purified by column chromatography (EA in PE=1˜50%) to give the title compound (57.0 g, 98.7%) as a yellow oil. ¹H NMR (400 MHz CDCl₃) δ 7.41-7.32 (m, 4H), 7.25-7.23 (m, 2H), 5.04 (d, J=12.4 Hz, 1H), 4.87 (s, 1H), 4.70 (d, J=12.4 Hz, 1H), 4.70-4.57 (m, 2H), 4.39-4.32 (m, 1H), 4.23-4.21 (m, 1H), 3.95 (dd, J=11.2, 2.0 Hz, 1H), 3.82 (dd, J=11.2, 3.6 Hz, 1H), 3.50 (s, 3H).

### Step 5: Synthesis of Compound 7

To a solution of compound 6 (57.0 g, 119 mmol) in THF (300 mL) was added bromo (ethynyl)magnesium (0.5 M, 475 mL) at −65° C. The mixture was stirred at −65° C. for 1 h and then was warmed to 15° C. for 15 h. The reaction was quenched by aq. NH₄Cl (250 mL), extracted by ethyl acetate (200 mL×2), the organic layers were combined and washed with brine, dried over Na₂SO₄. Evaporated, the residue was purified by column chromatography (EA in PE=1˜50%) to give the title compound (40.7 g, 67.7%) as a yellow solid. ¹H NMR (400 MHz CDCl₃) δ 7.41-7.33 (m, 4H), 7.26-7.22 (m, 2H), 4.89 (s, 1H), 4.85-4.65 (m, 4H), 4.44-4.33 (m, 2H), 3.95 (t, J=5.0 Hz, 1H), 3.72 (d, J=5.2 Hz, 2H), 3.52 (s, 3H), 3.50 (s, 1H), 2.59 (s, 1H).

### Step 6: Synthesis of Compound 8

To a solution of compound 7 (35.4 g, 69.9 mmol) in DMF (250 mL) was added NaH (3.36 g, 83.9 mmol, 60% purity) at 0° C., the mixture was stirred at 0° C. for 1 h. Then Mel (19.9 g, 140 mmol, 8.71 mL) and TBAI (5.17 g, 14.0 mmol) was added, the reaction mixture was stirred at 25° C. for 2 h. The reaction mixture was quenched with saturated NH₄Cl solution (20.0 mL) and extracted with EtOAc (100 mL×3), the combined organic layers were washed with brine (100 mL), dried over anhydrous Na₂SO₄, filtered and concentrated to give the title compound (35.0 g, crude) as yellow oil. ¹H NMR (400 MHz CDCl₃) δ 7.47 (d, J=8.4 Hz, 1H), 7.40 (d, J=8.4 Hz, 1H), 7.36 (d, J=2.0 Hz, 1H), 7.32 (d, J=2.0 Hz, 1H), 7.22 (dd, J=2.0, 8.4 Hz, 1H), 7.18 (dd, J=8.4, 2.0 Hz, 1H), 5.02 (s, 1H), 4.89 (d, J=13.6 Hz, 1H), 4.68 (d, J=13.6 Hz, 1H), 4.65-4.55 (m, 2H), 4.34-4.28 (m, 1H), 4.03 (d, J=4.8 Hz, 1H), 3.72 (d, J=5.2 Hz, 2H), 3.57 (s, 3H), 3.50 (s, 3H), 3.00-2.84 (m, 1H), 2.63 (s, 1H).

### Step 7: Synthesis of Compound 9

To a solution of compound 8 (23.8 g, 45.8 mmol) in AcOH (24.0 mL) and Ac₂O (48.0 mL) was added H₂SO₄ (4.49 g, 45.8 mmol, 2.44 mL). The mixture was stirred at 0° C. for 2 h. The reaction mixture was adjusted to pH=7 with saturated NaHCO₃at 0° C., and then extracted with EtOAc (300 mL×3). The combined organic layers were washed with brine (300 mL), dried over Na₂SO₄, filtered and concentrated under reduced pressure to give a residue which was purified by column chromatography (EA in PE=1˜50%) to give the title compound (17.1 g, 68.2%) as a white solid. ¹H NMR (400 MHz CDCl₃) δ 7.38-7.26 (m, 4H), 7.17-7.11 (m, 2H), 6.32-6.17 (m, 1H), 4.94-4.92 (m, 1H), 4.82-4.73 (m, 1H), 4.54-4.51 (m, 1H), 4.48-4.37 (m, 2H), 3.71 (dd, J=11.2, 2.8 Hz, 1H), 3.65 (dd, J=5.6, 2.8 Hz, 1H), 3.61-3.44 (m, 4H), 2.66-2.59 (m, 1H), 2.04-1.89 (m, 3H). MS (ESI) m/z [M+Na]⁺571.0.

### Step 8: Synthesis of Compound 10

To a solution of compound 9 (10.0 g, 18.2 mmol) and 1H-pyrimidine-2,4-dione (2.04 g, 18.2 mmol) in MeCN (250 mL) was added TMSOTf (11.8 g, 52.9 mmol, 9.56 mL) and DBU (6.94 g, 45.6 mmol, 6.87 mL) at 0° C., then stirred at 65° C. for 16 h. The reaction mixture was quenched with saturated NaHCO₃ (80.0 mL) and extracted with EtOAc (20.0 mL×3), dried over anhydrous Na₂SO₄, filtered and concentrated under reduced pressure to give a residue which was purified by column chromatography (EA in PE=5˜50%) to give the title compound (6.90 g, 63.0%) as a white solid. MS (ESI) m/z [M+Na]⁺601.1.

### Step 9: Synthesis of Compound 11

To a solution of compound 10 in DCM (100 mL) was added BCl₃ (1 M, 130 mL) at 0° C. The mixture was stirred at 0° C. for 16 h and then was quenched with MeOH (100 mL) and adjusted to pH=7 with saturated NH₃·H₂O (130 mL) at 0° C., and concentrated under reduced pressure to give a residue which was purified by column chromatography (EA in PE=3˜50%) to give the title compound (1.74 g, 47.4%) as white solid. ¹H NMR (400 MHz MeOD) δ 8.09-7.68 (m, 1H), 6.36-6.11 (m, 1H), 5.71-5.67 (m, 1H), 4.40-4.23 (m, 1H), 4.21-3.64 (m, 3H), 3.63-3.53 (m, 3H), 3.31-3.25 (m, 1H).

### Step 10 and 11: Synthesis of Example 95C

The title compound was obtained as diastereomeric mixture of 1′-alfa and 1′-beta isomers with similar procedures to those described for Example 8C(23 mg, 12.4% for two steps). ¹H NMR (400 MHz, D₂O) δ 8.07-7.71 (m, 1H), 6.41-6.09 (m, 1H), 5.94-5.79 (m, 1H), 5.61-5.43 (m, 2H), 5.29-5.15 (m, 2H), 5.11-5.01 (m, 1H), 4.51-4.22 (m, 5H), 4.11-3.95 (m, 2H), 3.54-3.31 (m, 3H), 3.18-3.04 (m, 1H), 2.17-2.16 (m, 3H), 2.08 (s, 3H), 2.00-1.96 (m, 6H), 1.93-1.91 (m, 3H). MS (ESI) m/z [M−H]⁻859.1.

### Example 96C

### Synthesis of Example 96C

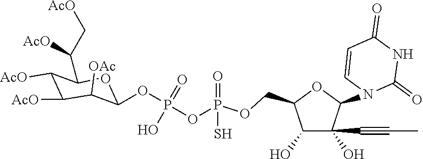 [SMILES: CC#C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1ccc(=O)[nH]c1=O]

The title compound was obtained with similar procedures to those described for Example 26C(77 mg, 37.6%) by using BrMgC≡CCH₃ instead of BrMgC≡C. ¹H NMR (400 MHz, D₂O) δ 8.05-7.99 (m, 1H), 6.00 (s, 1H), 5.93 (d, J=8.1 Hz, 1H), 5.61-5.54 (m, 2H), 5.27-5.20 (m, 2H), 5.11-5.04 (m, 1H), 4.50-4.41 (m, 1H), 4.39-4.33 (m, 1H), 4.32-4.25 (m, 1H), 4.25-4.15 (m, 2H), 4.14-4.04 (m, 2H), 2.22-2.16 (m, 3H), 2.10 (s, 3H), 2.01 (s, 3H), 2.00-1.97 (m, 3H), 1.94 (s, 3H), 1.74-1.68 (m, 3H). ³¹P NMR (162 MHz, D₂O) δ 43.87, −15.24. MS (ESI) m/z [M−H]⁻859.1.

### Example 96D

### Synthesis of Example 96D

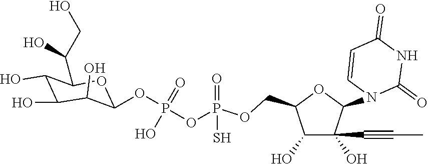 [SMILES: CC#C[C@@]1(O)[C@H](O)[C@@H](COP(=O)(S)OP(=O)(O)OC2OC([C@@H](O)CO)C(O)C(O)C2O)O[C@H]1n1ccc(=O)[nH]c1=O]

The title compound was obtained from Example 96C with similar procedures to those described for Example 1D as TEA salt (18.1 mg, 58.9%). ¹H NMR (400 MHz, D₂O) δ 8.05-7.96 (m, 1H), 6.01 (s, 1H), 5.93 (d, J=8.1 Hz, 1H), 5.27-5.20 (m, 2H), 4.43-4.33 (m, 1H), 4.28-4.17 (m, 2H), 4.15-4.04 (m, 2H), 3.93-3.87 (m, 1H), 3.80-3.60 (m, 4H), 3.38-3.31 (m, 1H), 3.14 (q, J=7.3 Hz, 11H), 1.70 (s, 3H), 1.22 (t, J=7.3 Hz, 16H). ³¹P NMR (162 MHz, D₂O) δ 43.51, −14.42. MS (ESI) m/z [M−H]⁻649.0.

### Example 97C

### Synthesis of Example 97C

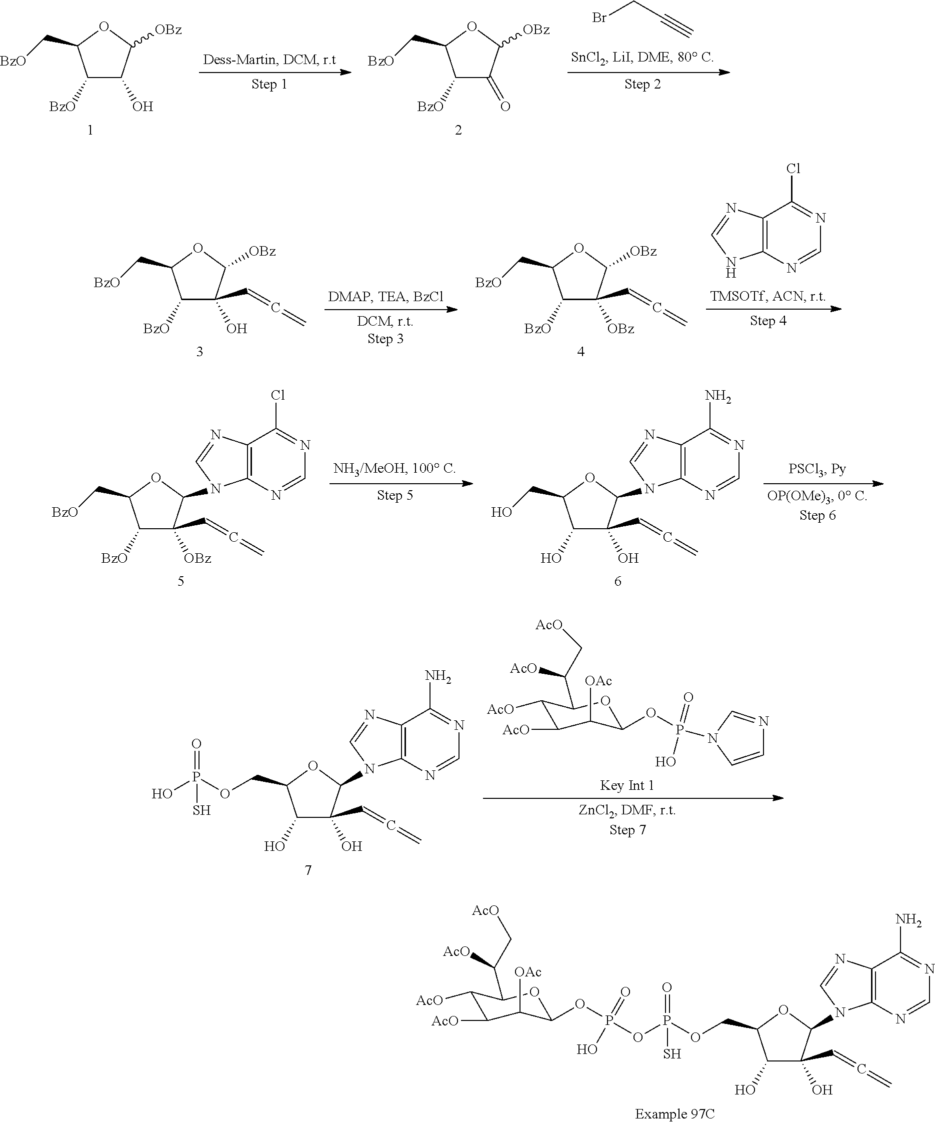

### Step 1: Synthesis of Compound 2

The title compound was obtained with the same procedures as described for Example 25C.

### Step 2: Synthesis of Compound 3

3-Bromoprop-1-yne (736.31 mg, 6.19 mmol) was added to a solution of compound 2 (1 g, 2.06 mmol), SnCl₂ (586.82 mg, 3.09 mmol) and LiI (552.29 mg, 4.13 mmol) in DME (15 mL) at r.t., the reaction mixture was stirred at 80° C. for 1 h. The crude product was purified by flash silica gel chromatography (0˜25% EA in PE) to give the title compound (780 mg, 72%) as an oil. ¹H NMR (400 MHz, CDCl₃) δ 8.15-8.05 (m, 6H), 7.66-7.53 (m, 3H), 7.49-7.40 (m, 6H), 6.52 (s, 1H), 5.62 (t, J=6.7 Hz, 1H), 5.45 (d, J=3.2 Hz, 1H), 5.15 (d, J=6.7 Hz, 2H), 4.85-4.79 (m, 1H), 4.70 (dd, J=11.9, 4.5 Hz, 1H), 4.65-4.57 (m, 1H). MS (ESI) m/z [M+Na]⁺522.9. Step 3: Compound 4 Benzoyl chloride (438.13 mg, 3.12 mmol) was added to a solution of compound 3 (0.78 g, 1.56 mmol), DMAP (190.39 mg, 1.56 mmol) and TEA (473.10 mg, 4.68 mmol, 651.65 μL) in DCM (20.18 mL). The reaction mixture was stirred at room temperature for 5 h. The residue was purified by column chromatography (0˜20% EA in PE) to afford the title compound (0.8 g, 84.9%) as a white solid. ¹H NMR (400 MHz, CDCl₃) δ 8.17 (dd, J=7.5, 6.2 Hz, 2H), 8.13-8.08 (m, 2H), 8.08-8.04 (m, 2H), 7.92 (dd, J=8.2, 1.1 Hz, 2H), 7.66-7.60 (m, 3H), 7.55-7.43 (m, 7H), 7.18 (dd, J=13.9, 6.2 Hz, 2H), 7.12 (s, 1H), 6.23 (d, J=7.7 Hz, 1H), 6.01 (t, J=6.8 Hz, 1H), 4.86-4.73 (m, 4H), 4.53 (dd, J=13.2, 6.0 Hz, 1H). MS (ESI) m/z [M+Na]⁺627.0.

### Step 4: Synthesis of Compound 5

Trimethylsilyl trifluoromethanesulfonate (441.13 mg, 1.98 mmol, 383.59 μL) was added to a solution of compound 4 (400 mg, 0.66 mmol) and 6-chloro-9H-purine (203 mg, 1.32 mmol) in Acetonitrile (10 mL). The reaction mixture was stirred at room temperature for 5 h. The residue was purified by column chromatography (0˜50% EA in PE) to afford the title compound (130 mg, 32%) as a white solid. MS (ESI) m/z [M+H]⁺637.0.

### Step 5: Synthesis of Compound 6

A solution of compound 5 (130 mg, 0.21 mmol) in NH₃ (7 M in MeOH, 3 mL) was stirred at 100° C. for 6 h. The solvent was removed under reduced pressure, the crude product was purified by column chromatography (0˜10% EA in MeOH) to afford the title compound (60 mg, 93%) as a white solid. ¹H NMR (400 MHz, MeOD) δ 8.50 (s, 1H), 8.18 (s, 1H), 6.13 (s, 1H), 4.73 (dd, J=11.5, 6.8 Hz, 1H), 4.55 (d, J=9.1 Hz, 1H), 4.44 (dd, J=11.5, 6.7 Hz, 1H), 4.07 (ddd, J=14.7, 11.2, 2.5 Hz, 3H), 3.89 (d, J=2.7 Hz, 1H). MS (ESI) m/z [M+H]⁺306.0.

### Step 6 and 7: Synthesis of Example 97C

The title compound was obtained with similar procedures to those described for Example 8C(30 mg, 11.4% for two steps). ¹H NMR (400 MHz, D₂O) δ 8.65-8.55 (m, 1H), 8.33-8.32 (m, 1H), 6.14-6.11 (m, 1H), 5.61-5.49 (m, 2H), 5.20-5.11 (m, 2H), 5.04-4.97 (m, 1H), 4.80-4.73 (m, 2H), 4.63-4.54 (m, 1H), 4.46-4.41 (m, 1H), 4.40-4.32 (m, 2H), 4.31-4.15 (m, 3H), 4.05-3.97 (m, 1H), 2.13-2.11 (m, 3H), 2.03 (s, 3H), 1.94 (s, 3H), 1.91-1.88 (m, 3H), 1.86 (s, 3H). ³¹P NMR (162 MHz, D₂O) δ 43.57, −15.24. MS (ESI) m/z [M−H]⁻881.7.

### Example 97D

### Synthesis of Example 97D

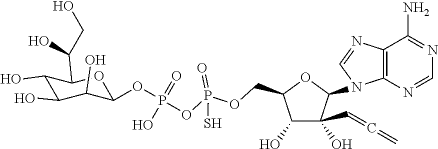 [SMILES: C=C=C[C@@]1(O)[C@H](O)[C@@H](COP(=O)(S)OP(=O)(O)OC2OC([C@@H](O)CO)C(O)C(O)C2O)O[C@H]1n1cnc2c(N)ncnc21]

Two isomers of the title compound were obtained from Example 97C with similar procedures to those described for Example 1D.

Isomer 1 (1.0 mg, 8.7%): ¹H NMR (400 MHz, D₂O) δ 8.64 (s, 1H), 8.34 (s, 1H), 6.19 (s, 1H), 5.20 (d, J=8.5 Hz, 1H), 4.58 (d, J=9.0 Hz, 2H), 4.43-4.39 (m, 2H), 4.32-4.24 (m, 3H), 4.03 (d, J=2.8 Hz, 1H), 3.87-3.83 (m, 1H), 3.74-3.60 (m, 3H), 3.56 (dd, J=9.7, 3.2 Hz, 1H), 3.27 (d, J=9.8 Hz, 1H). ³¹P NMR (162 MHz, D₂O) δ 43.47, −14.38. MS (ESI) m/z [M−H]⁻672.1.

Isomer 2 (1.5 mg, 13.1%): ¹H NMR (400 MHz, D₂O) δ 8.60 (s, 1H), 8.31 (s, 1H), 6.13 (s, 1H), 5.18 (d, J=8.5 Hz, 2H), 4.58-4.54 (m, 1H), 4.47 (d, J=9.2 Hz, 1H), 4.43-4.39 (m, 1H), 4.29-4.24 (m, 2H), 4.00 (d, J=3.1 Hz, 1H), 3.85-3.80 (m, 1H), 3.71-3.52 (m, 4H), 3.56 (dd, J=9.7, 3.2 Hz, 1H), 3.29-3.21 (m, 1H). ³¹P NMR (162 MHz, D₂O) δ 43.38, −14.30. MS (ESI) m/z [M−H]⁻672.1.

### Example 98C

### Synthesis of Example 98C

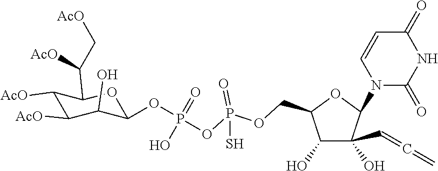 [SMILES: C=C=C[C@@]1(O)[C@H](O)[C@@H](COP(=O)(S)OP(=O)(O)OC2OC([C@H](COC(C)=O)OC(C)=O)C(OC(C)=O)C(OC(C)=O)C2O)O[C@H]1n1ccc(=O)[nH]c1=O]

The title compound was obtained with similar procedures to those described for Example 97C(3.7 mg, 10.8%) by using 1H-pyrimidine-2,4-dione instead of 6-chloro-9H-purine. ¹H NMR (400 MHz, D₂O) δ 7.95 (d, J=7.7 Hz, 1H), 6.02-5.84 (m, 2H), 5.60-5.50 (m, 2H), 5.24-5.17 (m, 2H), 5.12-4.98 (m, 2H), 4.97-4.90 (m, 1H), 4.88-4.81 (m, 1H), 4.47-4.31 (m, 2H), 4.29-4.01 (m, 5H), 2.15 (s, 3H), 2.06 (s, 3H), 1.97 (s, 3H), 1.96-1.93 (m, 3H), 1.90 (s, 3H). ³¹P NMR (162 MHz, D₂O) δ 43.46, −15.40. MS (ESI) m/z [M−H]⁻859.1.

### Example 108C

### Synthesis of Example 108C

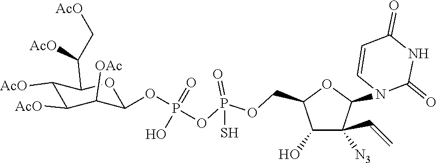 [SMILES: C=C[C@@]1(N=[N+]=[N-])[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1ccc(=O)[nH]c1=O]

The title compound was obtained with similar procedures to those described for Example 41C(42 mg, 23.5%) by using compound 7 in the preparation of Example 110C as the starting material. ¹H NMR (400 MHz, D₂O) δ 6.84-6.81 (m, 1H), 6.01-5.91 (m, 3H), 5.55-5.38 (m, 4H), 5.22-5.14 (m, 2H), 5.03-4.96 (m, 1H), 4.36-4.29 (m, 1H), 4.27-4.18 (m, 2H), 4.05-3.94 (m, 2H), 3.90-3.72 (m, 2H), 2.09 (s, 3H), 2.02 (s, 3H), 1.93 (s, 3H), 1.91 (s, 3H), 1.86 (s, 3H). ³¹P NMR (162 MHz, D₂O) δ 43.95, −15.21. MS (ESI) m/z [M−H]⁻872.1.

### Example 110C

### Synthesis of Example 110C

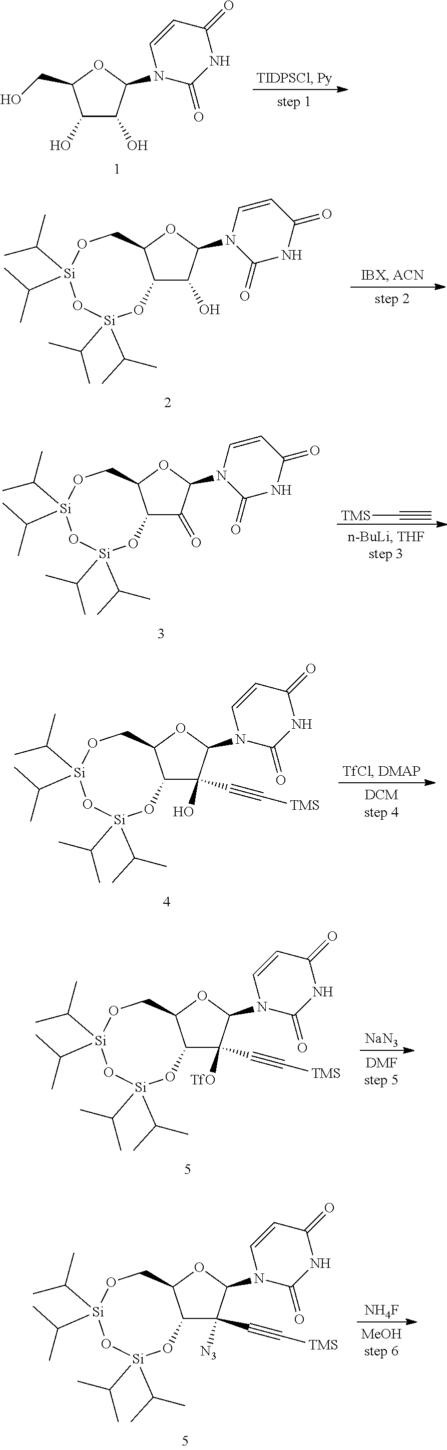

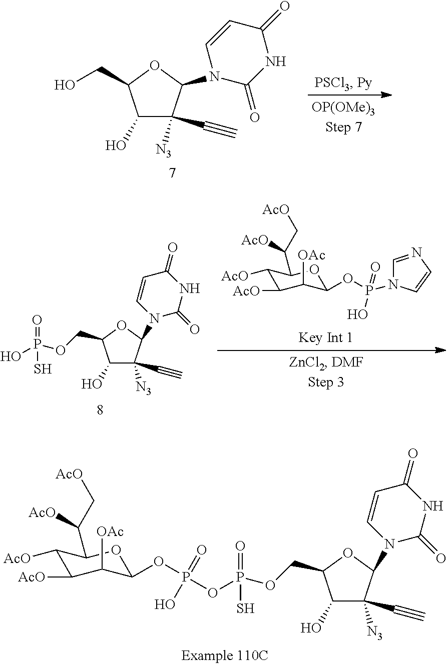 [SMILES: C#C[C@@]1(N=[N+]=[N-])[C@H](O)[C@@H](CO)O[C@H]1n1ccc(=O)[nH]c1=O ; "PSCl3, Py" ; C#C[C@@]1(N=[N+]=[N-])[C@H](O)[C@@H](COP(=O)(O)S)O[C@H]1n1ccc(=O)[nH]c1=O ; CC(=O)OC[C@H](OC(C)=O)C1OC(OP(=O)(O)n2ccnc2)C(OC(C)=O)C(OC(C)=O)C1OC(C)=O ; C#C[C@@]1(N=[N+]=[N-])[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1ccc(=O)[nH]c1=O]

### Step 1: Synthesis of Compound 2

To a solution of compound 1 (25 g, 0.102 mol) in dry Pyridine (100 mL) was added TIDPSCl (32.3 g, 0.102 mol). The resulting solution was stirred at rt for 4 h. Then the solvent was removed in vacuo. The crude product was purified by silica gel column chromatography (EA/PE=0˜40%) to give the title compound (33 g, 62.8%) as a white solid. MS (ESI) m/z [M+H]⁺486.9.

### Step 2: Synthesis of Compound 3

To a solution of compound 2 (33 g, 67.8 mmol) in dry ACN (160 mL) was added IBX (37.9 g, 135.6 mmol). The resulting solution was stirred at 80° C. for 5 h. The solid was filtered out. The filtrate was concentrated under vacuum to give the title compound (33g, crude), which was used for the next step without further purification. MS (ESI) m/z [M+H]⁺485.0. Step 3: Synthesis of Compound 4

To a solution of ethynyltrimethylsilane (10.1 g, 103.1 mmol) in THF (100 mL) was added n-BuLi (43 mL, 2.4M) at −78° C., stirred for 30 min at −78° C., then stirred for 30 min at −55° C. A solution of compound 3 (10 g, 20.63 mmol) in THF (40 mL) was added, the reaction was stirred for 2h. Sat. aqueous NH₄Cl (100 mL) was added to the mixture, extracted with EA (100 mL×2). The organic layers were combined and dried over anhydrous Na₂SO₄, filtered, and the filtrate was concentrated and purified by flash chromatography (EA/PE=0˜60%) to give the title compound (5 g, 41.5%) as a brown oil. ¹H NMR (400 MHz, DMSO-d₆) δ 11.41-11.34 (m, 1H), 7.45 (d, J=8.2 Hz, 1H), 6.57 (s, 1H), 6.03 (s, 1H), 5.55 (dd, J=8.1, 2.1 Hz, 1H), 4.09 (d, J=7.6 Hz, 1H), 4.00-3.95 (m, 1H), 3.82-3.72 (m, 1H), 1.09-1.00 (m, 28H), 0.18-0.11 (m, 9H). MS (ESI) m/z [M+H]⁺583.3.

### Step 4: Synthesis of Compound 5

To a mixture of compound 4 (5 g, 8.5 mmol) and DMAP (3.14 g, 25.7 mmol) in DCM (50 mL) was added trifluoromethanesulfonyl chloride (1.4 g, 8.6 mmol) at 0° C., the resulting mixture was stirred at 0° C. for 3h. Partition the reaction between ice cold 1% AcOH (200 mL) and DCM (200 mL). The organic phase was washed with ice cold saturated NaHCO₃ (100 mL) and ice cold saturated NaCl (100 mL), dried over anhydrous Na₂SO₄, filtered. The filtrate was concentrated and the residue was purified by silica gel column chromatography (MeOH/DCM=0˜20%) to give the title compound (5 g,73%) as a yellow oil. ¹H NMR (400 MHz, MeOD) δ 8.28 (d, J=7.4 Hz, 1H), 6.89 (d, J=7.5 Hz, 1H), 6.13 (s, 1H), 4.03-3.88 (m, 1H), 3.84-3.80 (m, 1H), 3.27-3.17 (m, 1H), 3.07-2.98 (m, 1H), 1.00-0.83 (m, 28H), 0.04-−0.06 (m, 9H).

### Step 5: Synthesis of Compound 6

To a solution of compound 5 (5 g, 6.99 mmol) in DMF (40 mL) was added azidosodium (2.27 g, 34.96 mmol) at 25° C., the reaction was stirred for 3h. Quenched with water (100 mL), extracted with EA (150 mL). The organic layer was dried over anhydrous Na₂SO₄, filtered, and the filtrate was concentrated and purified by flash chromatography (MeOH/DCM=0˜20%) to give the title compound (4 g, 84.7%) as a brown oil. MS (ESI) m/z [M+H]⁺608.3.

### Step 6: Synthesis of Compound 7

To a solution of compound 6 (3.5 g, 5.76 mmol) in MeOH (30 mL) was added Ammonium fluoride (2.13 g, 57.5 mmol). The resulting solution was stirred at 70° C. for 1 h. Then the solvent was removed in vacuo. The crude product was purified by CombiFlash to give the title compound (1.92 g, 96.6%) as a brown solid. ¹H NMR (400 MHz, DMSO-d₆) δ 8.26 (s, 1H), 7.18-7.11 (m, 1H), 7.00-6.95 (m, 1H), 5.02-4.97 (m, 1H), 4.63-4.58 (m, 1H), 4.20-4.17 (m, 1H), 3.40-3.34 (m, 2H), 2.88 (s, 1H). MS (ESI) m/z [M+H]⁺294.0.

### Step 7 and 8: Synthesis of Example 110C

The title compound was obtained with similar procedures to those described for Example 8C(20 mg, 6.7% for two steps). ¹H NMR (400 MHz, D₂O) δ 6.92 (d, J=8.0 Hz, 1H), 6.13 (s, 1H), 6.12-6.03 (m, 1H), 5.65-5.50 (m, 2H), 5.27 (d, J=7.2 Hz, 2H), 5.08 (t, J=9.9 Hz, 1H), 4.44-4.36 (m, 2H), 4.35-4.27 (m, 1H), 4.12 (d, J=9.7 Hz, 1H), 4.08-4.02 (m, 1H), 3.98-3.91 (m, 1H), 3.90-3.82 (m, 1H), 3.47 (s, 1H), 2.18 (s, 3H), 2.11 (s, 3H), 2.02 (s, 3H), 2.00 (s, 3H), 1.94 (s, 3H). MS (ESI) m/z [M−H]⁻870.1.

### Example 112C

### Synthesis of Example 112C

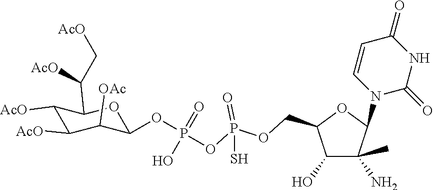 [SMILES: CC(=O)OC[C@H](OC(C)=O)C1OC(OP(=O)(O)OP(=O)(S)OC[C@H]2O[C@@H](n3ccc(=O)[nH]c3=O)[C@](C)(N)[C@@H]2O)C(OC(C)=O)C(OC(C)=O)C1OC(C)=O]

The title compound was obtained from Example 104C with similar procedures to those described for Example 33C(16 mg, 38.4%) as white solid. ¹H NMR (400 MHz, D₂O) δ 7.94 (dd, J=8.2, 1.8 Hz, 1H), 6.12 (s, 1H), 5.93-5.90 (m, 1H), 5.61-5.52 (m, 2H), 5.26-5.16 (m, 2H), 5.08-5.02 (m, 1H), 4.43-5.37 (m, 1H), 4.34-4.27 (m, 2H), 4.25-4.22 (m, 1H), 4.21-4.16 (m, 1H), 4.15-4.11 (m, 1H), 4.09-4.04 (m, 1H), 2.16 (s, 3H), 2.07-2.06 (m, 3H), 1.98 (s, 3H), 1.97-1.96 (m, 3H), 1.91 (s, 3H), 1.28 (s, 3H). ³¹P NMR (162 MHz, D₂O) δ 43.78, −15.22. MS (ESI) m/z [M−H]⁻834.1.

### Example 120C

### Synthesis of Example 120C

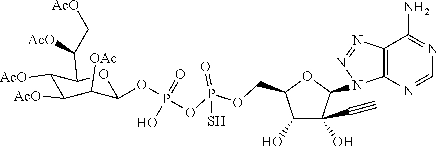 [SMILES: C#C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1nnc2c(N)ncnc21]

The title compound was obtained with similar procedures to those described for Example 25C(25 mg, 74.3%) by using 3H-[1,2,3]triazolo[4,5-d]pyrimidin-7-amine instead of 6-chloro-9H-purine. ¹H NMR (400 MHz, D₂O) δ 8.49 (s, 1H), 6.48 (s, 1H), 5.62-5.54 (m, 2H), 5.23-5.15 (m, 2H), 5.08-5.01 (m, 1H), 4.93-4.85 (m, 1H), 4.47-4.32 (m, 4H), 4.31-4.23 (m, 1H), 4.05 (d, J=10.0 Hz, 1H), 2.61 (d, J=5.2 Hz, 1H), 2.16 (s, 3H), 2.10-2.07 (m, 3H), 2.00 (s, 3H), 1.96 (s, 3H), 1.92 (s, 3H). MS (ESI) m/z [M−H]⁻869.0.

### Example 120D

### Synthesis of Example 120D

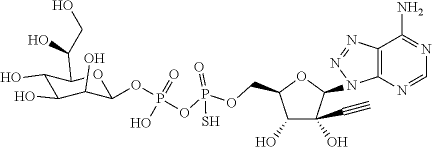 [SMILES: C#C[C@@]1(O)[C@H](O)[C@@H](COP(=O)(S)OP(=O)(O)OC2OC([C@@H](O)CO)C(O)C(O)C2O)O[C@H]1n1nnc2c(N)ncnc21]

The title compound was obtained from Example 120C with similar procedures to those described for Example 1D (0.9 equiv. of TEA salt, 3.6 mg, 23.7%). ¹H NMR (400 MHz, D₂O) δ 8.36 (s, 1H), 6.46 (s, 1H), 5.23-5.15 (m, 1H), 4.95-4.86 (m, 1H), 4.51-4.36 (m, 3H), 4.06 (dd, J=9.2, 2.8 Hz, 1H), 3.89-3.82 (m, 1H), 3.76-3.60 (m, 3H), 3.58-3.47 (m, 1H), 3.29-3.19 (m, 1H), 3.13 (q, J=7.2 Hz, 6H), 2.58 (s, 1H), 1.21 (t, J=7.2 Hz, 8H). 1P NMR (162 MHz, D₂O) δ 43.56, −14.52. MS (ESI) m/z [M−H]⁻659.1.

### Example 122C

### Synthesis of Example 122C

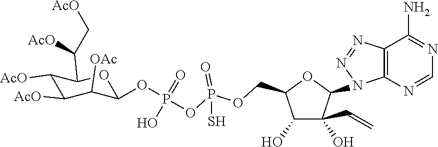 [SMILES: C=C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1nnc2c(N)ncnc21]

The title compound was obtained with similar procedures to those described for Example 31C(55 mg, 41%) by using 3H-[1,2,3]triazolo[4,5-d]pyrimidin-7-amine instead of 6-chloro-9H-purine. ¹H NMR (400 MHz, D₂O) δ 8.43 (s, 1H), 6.33 (s, 1H), 5.63-5.53 (m, 2H), 5.44-5.37 (m, 1H), 5.22-5.08 (m, 4H), 5.06-5.01 (m, 1H), 4.83-4.77 (m, 1H), 4.50-4.34 (m, 4H), 4.30-4.22 (m, 1H), 4.04 (dd, J=10.0, 1.6 Hz, 1H), 2.16 (s, 3H), 2.10-2.06 (m, 3H), 2.01-1.98 (m, 3H), 1.97-1.94 (m, 3H), 1.93-1.90 (m, 3H). 1P NMR (162 MHz, D₂O) δ 43.90-43.45, −15.16-−15.34. MS (ESI) m/z [M−H]⁻871.1.

### Example 122D

### Synthesis of Example 122D

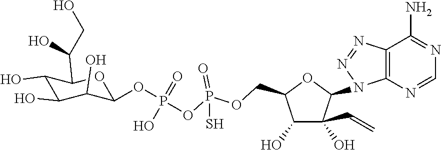 [SMILES: C=C[C@@]1(O)[C@H](O)[C@@H](COP(=O)(S)OP(=O)(O)OC2OC([C@@H](O)CO)C(O)C(O)C2O)O[C@H]1n1nnc2c(N)ncnc21]

The title compound was obtained from Example 122C with similar procedures to those described for Example 1D (0.7 equiv. of TEA salt, 9 mg, 26.4%). ¹H NMR (400 MHz, D₂O) δ 8.42 (s, 1H), 6.33 (s, 1H), 5.44-5.36 (m, 1H), 5.24-5.07 (m, 2H), 4.88-4.80 (m, 1H), 4.51-4.36 (m, 3H), 4.09-4.04 (m, 1H), 3.89-3.83 (m, 1H), 3.77-3.54 (m, 4H), 3.32-3.23 (m, 1H), 3.13 (q, J=7.2 Hz, 4H), 1.20 (t, J=7.2 Hz, 6H). 1P NMR (162 MHz, D₂O) δ 43.41, −14.40. MS (ESI) m/z [M−H]⁻661.0.

### Example 125C

### Synthesis of Example 125C

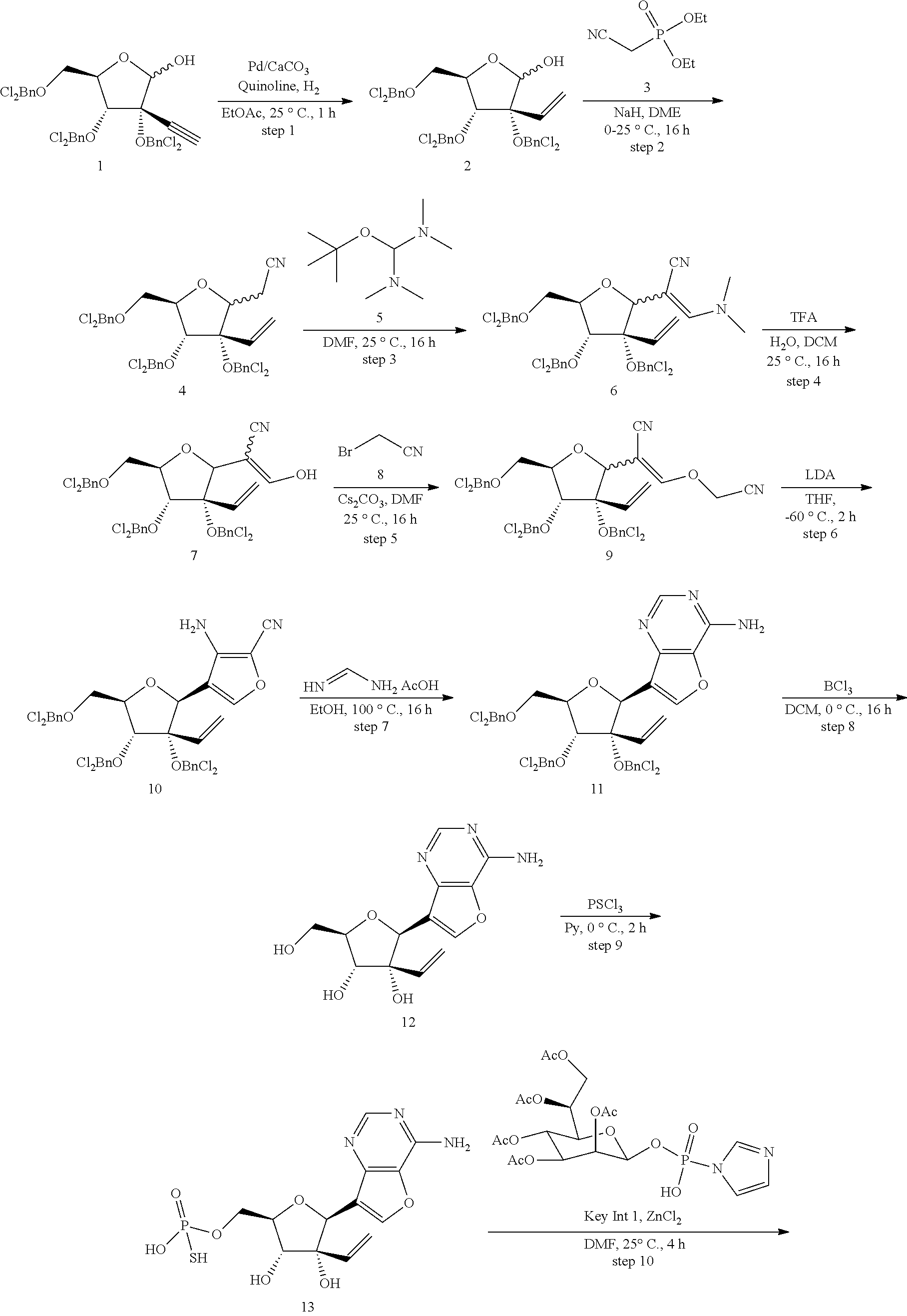

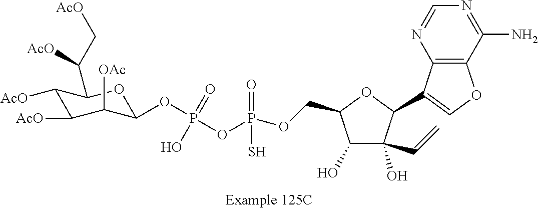 [SMILES: C=C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1coc2c(N)ncnc12]

### Step 1: Synthesis of Compound 2

To a suspension of Pd/CaCO₃ (6.34 g, 30.7 mmol) in EA (400 mL) was added compound 1 (40.0 g, 61.4 mmol) and quinoline (15.9 g, 123 mmol). The reaction was degassed and purged with H₂ for 3 times, then stirred at 25° C. for 1 h under H₂ (15 Psi) atmosphere. The reaction mixture was then filtered and concentrated under reduced pressure. The residue was purified by column chromatography on silica gel (PE/EA=100/1 to 0/1) to give the title compound 2 (30.0 g, 45.9 mmol, 74.8% yield, 90.0% purity) as a yellow oil. MS (ESI) m/z [M+H₂O]⁺670.2.

### Step 2: Synthesis of Compound 4

To a solution of compound 3 (14.2 g, 80.4 mmol) in DME (280 mL) was added NaH (3.21 g, 80.4 mmol, 60% w/w) at 0° C. The reaction mixture was stirred for 0.5 h at 0° C., followed by the addition of a solution of compound 2 (35.0 g, 53.6 mmol) in DME (70.0 mL) dropwise. The reaction mixture was stirred for 15.5 h at 25° C., then quenched by icy aq. NH₄Cl (200 mL) and extracted with EA (150 mL×3). The combined organic layers were washed with brine (300 mL), dried over Na₂SO₄, filtered and concentrated under reduced pressure. The residue was purified by column chromatography on silica gel (PE/EA=4/1) to give the title compound 4 (33.0 g, 91.1% yield) as a yellow solid. MS (ESI) m/z [M+Na]⁺695.0. Step 3: Synthesis of Compound 6

To a solution of compound 4 (33.0 g, 48.8 mmol) in DMF (330 mL) was added compound (42.5 g, 244 mmol). The resulting solution was stirred for 16 h at 25° C. The reaction mixture was quenched by water (1.0 L) at 25° C. and extracted with EA (600 mL×3). The combined organic layers were washed with brine (1.2 L), dried over Na₂SO₄, filtered and concentrated under reduced pressure. The residue was purified by column chromatography on silica gel (PE/EA=3/1) to give the title compound 6 (23.0 g, 59.9% yield, mixture of isomers) as a white solid. ¹H NMR (400 MHz, CDCl₃) δ 7.77-7.27 (m, 6H), 7.26-7.13 (m, 3H), 6.63-6.42 (m, 1H), 5.99-5.95 (m, 1H), 5.55-5.42 (m, 2H), 5.13-4.42 (m, 7H), 4.37-4.26 (m, 2H), 3.91-3.68 (m, 2H), 3.08 (d, J=8.2 Hz, 6H). MS (ESI) m/z [M+H]⁺731.2.

### Step 4: Synthesis of Compound 7

To a solution of compound 6 (20.0 g, 27.4 mmol) in DCM (200 mL) was added TFA (40 mL) and water (160 mL). The reaction mixture was stirred at 25° C. for 16 h. The reaction mixture was extracted with DCM (150 mL×2). The combined organic layers were dried over Na₂SO₄, filtered and concentrated under reduced pressure to give the title compound 7 (21.0 g, crude) as a yellow solid. MS (ESI) m/z [M+H]⁺704.1.

### Step 5: Synthesis of Compound 9

To a solution of compound 7 (10.0 g, 14.2 mmol) in DMF (100 mL) was added compound 8 (5.11 g, 42.6 mmol) and Cs2CO₃ (10.2 g, 31.2 mmol). The reaction mixture was stirred at 25° C. for 16 h. Two reactions were carried out and combined for workup. The combined reaction mixture was quenched by aq. NH₄Cl (500 mL) at 25° C., then diluted with water (300 mL) and extracted with EA (600 mL×2). The combined organic layers were washed with brine (900 mL), dried over Na₂SO₄, filtered and concentrated under reduced pressure to give the title compound 9 (21.0 g, crude) as a yellow oil. MS (ESI) m/z [M+H]⁺743.2.

### Step 6: Synthesis of Compound 10

To a solution of compound 9 (20.0 g, 26.9 mmol) in THF (200 mL) was added LDA (2.00 M, 20.2 mL, 40.2 mmol) dropwise at −60° C. The reaction mixture was stirred at −60° C. for 2 h, then quenched by icy water (1.0 L) at 0° C. and extracted with EA (600 mL×2). The combined organic layers were concentrated under reduced pressure. The residue was purified by column chromatography on silica gel (PE/EA=4/1) to give the title compound 10 (3.00 g, 24.1% yield, 80.3% purity) as a yellow solid. ¹H NMR (400 MHz, CDCl₃) δ 7.45-7.42 (m, 1H), 7.41-7.37 (m, 2H), 7.36-7.35 (m, 1H), 7.33 (m, 1H), 7.29 (s, 1H), 7.25 (m, 1H), 7.20-7.14 (m, 3H), 5.72-5.63 (m, 1H), 5.40-5.30 (m, 2H), 5.09 (s, 1H), 4.77 (m, 1H), 4.74-4.68 (m, 2H), 4.66 (m, 2H), 4.63-4.59 (m, 2H), 4.41-4.37 (m, 1H), 4.37-4.33 (m, 1H), 4.31 (s, 1H), 3.88-3.74 (m, 2H).

### Step 7: Synthesis of Compound 11

To a solution of compound 10 (2.00 g, 2.69 mmol) in EtOH (20 mL) was added acetic acid and methanimidamide (8.40 g, 80.7 mmol). The reaction mixture was stirred at 100° C. for 16 h and then concentrated under reduced pressure. The residue was purified by column chromatography on silica gel (PE/EA=1/2) to give the title compound 11 (0.80 g, 37.4% yield, 96.8% purity) as a yellow solid. ¹H NMR (400 MHz, DMSO-d₆) δ 8.24 (s, 1H), 8.11 (s, 1H), 7.73 (d, J=8.4 Hz, 1H), 7.62-7.52 (m, 4H), 7.48-7.34 (m, 6H), 5.82-5.73 (m, 2H), 5.34 (s, 1H), 5.27-5.12 (m, 2H), 4.90-4.84 (m, 1H), 4.78-4.70 (m, 4H), 4.68-4.58 (m, 2H), 4.34-4.28 (m, 1H), 3.93-3.81 (m, 2H), 1.91 (s, 1H). MS (ESI) m/z [M+H]⁺770.2.

### Step 8: Synthesis of Compound12

To a solution of compound 11 (0.90 g, 1.17 mmol) in DCM (9 mL) was added BCl₃ (1 M, 11.7 mL, 11.7 mmol) dropwise at 0° C. The reaction mixture was stirred at 0° C. for 16 h, then quenched with MeOH (10.0 mL) and adjusted to pH-7 with sat. NH₃H₂O 2O (2 mL) at 0° C., concentrated under reduced pressure. The residue was purified by prep-HPLC(column: Welch Xtimate C18, 250×100 mm #10 um; mobile phase: [water (NH₄HCO₃)-CAN]; B %: 1%-20%, 20 min) to give the title compound 12 (73.6 mg, 21.5% yield) as a yellow solid. ¹H NMR (400 MHz, D₂O) δ 8.20 (br s, 1H), 8.00 (br s, 1H), 5.63-5.49 (m, 1H), 5.31 (m, 1H), 5.20 (br s, 1H), 5.04 (m, 1H), 4.28 m, 1H), 4.15-3.97 (m, 2H), 3.92-3.82 (m, 1H). MS (ESI) m/z [M+H]⁺294.1.

### Step 9 and 10: Synthesis of Example 125C

The title compound was obtained from compound 12 with similar procedures to those described for Example 8C(isomers, dr=1:1). ¹H NMR (400 MHz, D₂O) δ 8.63 (d, J=1.6 Hz, 2H), 8.38 (d, J=1.0 Hz, 1H), 8.36 (d, J=1.0 Hz, 1H), 5.73-5.60 (m, 7H), 5.47-5.41 (m, 2H), 5.37-5.27 (m, 4H), 5.26-5.09 (m, 6H), 4.51-4.46 (m, 4H), 4.41-4.33 (m, 3H), 4.32-4.22 (m, 4H), 4.20-4.15 (m, 2H), 2.24 (s, 6H), 2.16 (s, 3H), 2.15 (s, 3H), 2.08 (s, 3H), 2.07 (s, 3H), 2.05 (s, 3H), 2.03 (s, 3H), 2.00 (s, 3H), 1.99 (s, 3H). ³¹P NMR (150 MHz, D₂O) δ 44.19, −15.06,-15.26. MS (ESI) m/z [M−H]⁻870.0.

### Example 126C

### Synthesis of Example 126C

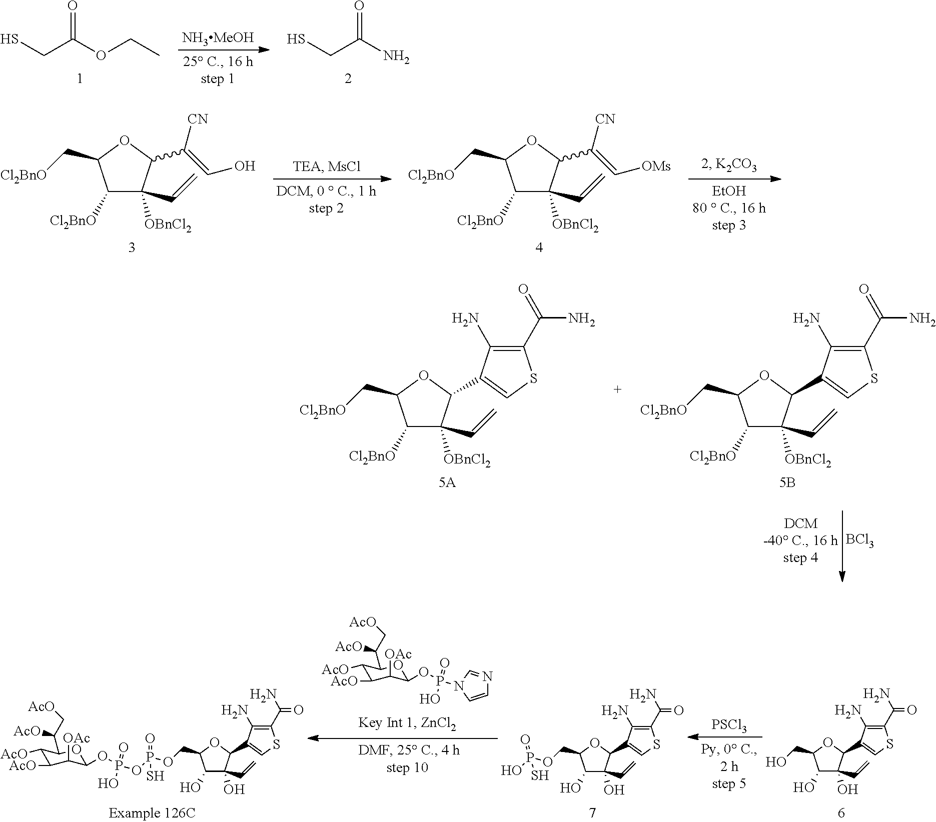

### Step 1: Synthesis of Compound 2

A solution of compound 1 (10.0 g, 94.2 mmol) in NH₃·MeOH (100 mL) was stirred for 16 h at 25° C. and then concentrated under reduced pressure. The residue was triturated with isopropyl ether (200 mL) at 20° C. for 30 min and then filtered to give the title compound 2 (5.00 g, 58.2% yield) as a white solid. ¹H NMR (400 MHz, DMSO-d₆) δ 7.64-7.35 (m, 1H), 7.04 (br s, 1H), 3.06 (s, 2H), 2.78 (br s, 1H)

### Step 2: Synthesis of Compound 4

To a solution of compound 3 (10.0 g, 14.2 mmol) in DCM (300 mL) was added TEA (4.31 g, 42.6 mmol) and MsCl (2.44 g, 21.3 mmol) at 0° C. The reaction mixture was stirred at 0° C. for 1 h, then diluted with water (200 mL) and extracted with DCM (200 mL×2). The organic phase was concentrated under reduced pressure to give the title compound 4 (11.0 g, crude) as a yellow oil. MS (ESI) m/z [M+H]⁺782.2.

### Step 3: Synthesis of Compound 5A and 5B

To a solution of compound 4 (11.0 g, 14.0 mmol) and compound 2 (2.56 g, 28.1 mmol) in EtOH (100 mL) was added K₂CO₃ (9.72 g, 70.3 mmol). The resulting solution was stirred for 16 h at 80° C., and then concentrated under reduced pressure. The residue was diluted with water (500 mL), extracted with EA (500 mL×2). The organic layers were concentrated and purified by column chromatography on silica gel (PE/EA=1/1) to give the title compound 5A (2.50 g, 45.7% yield) and compound 5B (2.50 g, 3.22 mmol, 45.7% yield) as a yellow solid. Compound 5B: ¹H NMR (400 MHz, DMSO-d₆) δ 7.70 (d, J=2.0 Hz, 1H), 7.68-7.62 (m, 2H), 7.62-7.58 (m, 2H), 7.51 (dd, J=2.0, 8.2 Hz, 1H), 7.47-7.40 (m, 2H), 7.40-7.35 (m, 2H), 6.99 (br s, 2H), 6.24 (br s, 2H), 5.68 (dd, J=11.2, 17.6 Hz, 1H), 5.40-5.29 (m, 2H), 5.18 (s, 1H), 4.76-4.65 (m, 6H), 4.48 (d, J=6.2 Hz, 1H), 4.40-4.29 (m, 1H), 3.96-3.83 (m, 2H).

### Step 4: Synthesis of Compound 6

The title compound was obtained from compound 5B with similar procedures to those described for compound 12 in the synthesis of Example 125C, which was carried out at a temperature of −40° C.(150 mg, 15.5% yield). ¹H NMR (400 MHz, D₂O) δ 7.41 (s, 1H), 5.53 (dd, J=10.8, 17.2 Hz, 1H), 5.34-5.23 (m, 1H), 5.06 (d, J=10.8 Hz, 1H). MS (ESI) m/z [M+H]⁺301.0.

### Step 5 and 6: Synthesis of Example 126C

The title compound was obtained from compound 6 with similar procedures to those described for Example 8C(isomers, dr=5:4). ¹H NMR (400 MHz, D₂O) δ 7.71 (d, J=0.9 Hz, 1H), 7.70 (d, J=0.9 Hz, 1H), 5.71-5.54 (m, 7H), 5.42-5.33 (m, 3H), 5.27-5.14 (m, 7H), 5.12-4.97 (m, 4H), 4.48-4.38 (m, 4H), 4.37-4.26 (m, 3H), 4.21 (s, 4H), 3.89-3.79 (m, 2H), 2.24 (s, 6H), 2.14 (s, 3H), 2.13 (s, 3H), 2.04-2.03 (m, 9H), 2.03 (s, 3H), 2.00 (s, 6H). ³¹P NMR (150 MHz, D₂O) δ 43.75, −15.34. MS (ESI) m/z [M−H]⁻877.0.

### Example 127C

### Synthesis of Example 127C

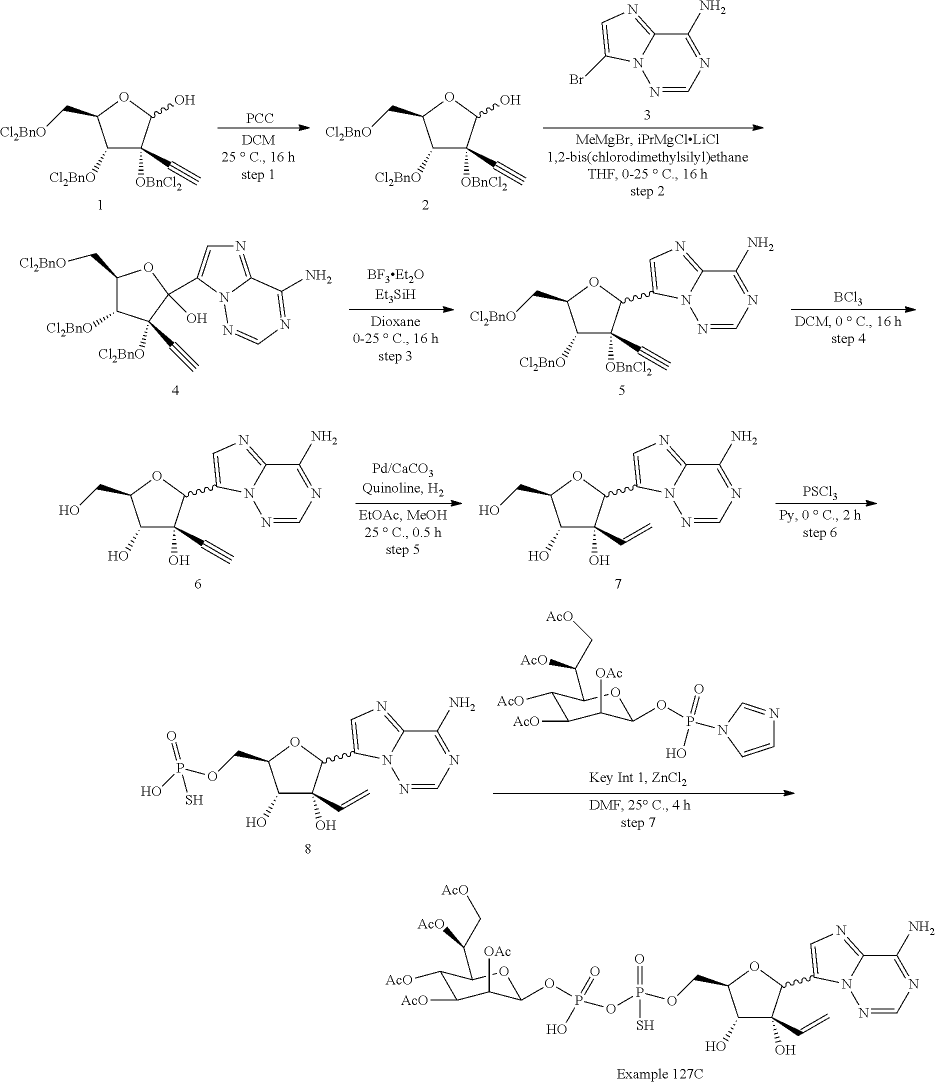

### Step 1: Synthesis of Compound 2

To a solution of compound 1 (54.0 g, 82.9 mmol) in DCM (1.0 L) was added PCC (39.3 g, 182. mmol). The reaction mixture was stirred for 16 h at 25° C., and then filtered through a pad of Celite. The filtrate was dried over Na₂SO₄, filtered and concentrated under reduced pressure. The residue was purified by column chromatography on silica gel (PE/EA=4/1) to give the title compound 2 (37 g, 68.7% yield) as a yellow oil. ¹H NMR (400 MHz, CDCl₃) δ 7.40-7.35 (m, 4H), 7.35-7.29 (m, 2H), 7.27-7.22 (m, 2H), 7.21-7.17 (m, 1H), 5.13-4.95 (m, 2H), 4.82 (dd, J=9.8, 12.0 Hz, 2H), 4.66-4.50 (m, 4H), 3.92 (dd, J=2.2, 11.6 Hz, 1H), 3.76 (dd, J=3.4, 11.6 Hz, 1H), 2.93 (s, 1H).

### Step 2: Synthesis of Compound 4

To a solution of compound 3 (6.00 g, 28.0 mmol) in THF (100 mL) was added MeMgBr (3.00 M, 10.3 mL, 30.9 mmol) at 0° C. The reaction mixture was stirred for 30 min at 0° C., followed by the addition of 1,2-Bis(chlorodimethylsilyl) ethane (6.64 g, 30.8 mmol) at 0° C. After being stirred for 30 min at 0° C., extra MeMgBr (3.00 M, 10.3 mL, 30.9 mmol) was added. The resulting mixture was stirred for further 30 min at 0° C., followed by the addition of iPrMgCl-LiCl (1.30 M, 23.7 mL, 30.9 mmol) at 0° C. After being stirred for further 1.5 h at 0° C., compound 2 (20.0 g, 30.8 mmol) was added to the reaction mixture at 0° C. and stirred at 25° C. for 16 h, then quenched by aq. NH₄Cl (200 mL), extracted with EA (200 mL×2). The combined organic layers were dried over Na₂SO₄, filtered and concentrated under reduced pressure. The residue was purified by column chromatography on silica gel (PE/EA=1/3) to give the title compound 4 (7.30 g, 33.2% yield) as a yellow solid. ¹H NMR (400 MHz, CDCl₃) δ 8.08 (s, 1H), 7.73-7.69 (m, 1H), 7.54-7.31 (m, 6H), 7.24-7.07 (m, 5H), 5.12-5.02 (m, 2H), 4.94-4.79 (m, 3H), 4.71-4.47 (m, 5H), 3.95-3.79 (m, 2H). MS (ESI) m/z [M+H]⁺806.1.

### Step 3: Synthesis of Compound 5

To a solution of compound 4 (5.50 g, 7.01 mmol) and Et₃SiH (4.97 g, 42.8 mmol) in dioxane (201 mL) was added BF₃·Et₂O (9.95 g, 70.1 mmol) at 0° C. The reaction mixture was stirred at 25° C. for 16 h, and then quenched by sat. aq. NaHCO₃ (50 mL), extracted with EA (50.0 mL×2). The organic layers were dried over Na₂SO₄, filtered and concentrated under reduced pressure. The residue was purified by column chromatography on silica gel (DCM/MeOH=1/3) to give the title compound 5 (2.60 g, 48.3% yield) as a white solid. ¹H NMR (400 MHz, CDCl₃) δ 8.25-8.12 (m, 1H), 7.83-7.78 (m, 1H), 7.63-7.50 (m, 1H), 7.46-7.28 (m, 5H), 7.27-7.14 (m, 3H), 5.97-5.80 (m, 1H), 5.12-4.79 (m, 4H), 4.73-4.50 (m, 3H), 4.44-4.33 (m, 1H), 3.97-3.72 (m, 2H), 2.78-2.42 (m, 1H). MS (ESI) m/z [M+H]⁺768.1.

### Step 4: Synthesis of Compound 6

The title compound was obtained from compound 5 with similar procedures to those described for compound 12 in the synthesis of Example 125C(500 mg, 94.2% yield). ¹H NMR (400 MHz, DMSO-d₆) δ 8.09 (s, 1H), 7.84-7.69 (m, 1H), 6.19-5.97 (m, 1H), 5.57-5.33 (m, 2H), 4.33-4.13 (m, 1H), 3.84-3.50 (m, 2H), 3.35-3.13 (m, 1H). MS (ESI) m/z [M+H]⁺292.2.

### Step 5: Synthesis of Compound 7

To a solution of compound 6 (300 mg, 1.03 mmol) and quinoline (133 mg, 1.03 mmol) in EA (15 mL) and MeOH (15 mL) was added Pd/CaCO₃ (127 mg, 618 μmol). The suspension was degassed and purged with H₂ for 3 times. The reaction mixture was stirred at 25° C. under H₂ (15.0 Psi) atmosphere for 30 min. The reaction mixture was then filtered and concentrated under reduced pressure. The residue was purified by prep-HPLC(column: Waters Xbridge BEH C18, 100×30 mm #10 um; mobile phase: [water (NH₄HCO₃)-ACN]; B %: 1%-18%, 7 min) to give the title compound 7 (500 mg, 94.2% yield) as a white solid. ¹H NMR (400 MHz, D₂O) δ 7.95 (s, 1H), 7.60 (s, 1H), 5.47-5.35 (m, 2H), 5.31-5.19 (m, 1H), 4.96 (d, J=10.8 Hz, 1H), 4.25 (d, J=8.8 Hz, 1H), 4.10-4.02 (m, 1H), 3.99-3.90 (m, 1H), 3.85-3.77 (m, 1H). MS (ESI) m z [M+H]⁺294.2.

### Step 6 and 7: Synthesis of Example 127C

The title compound was obtained with similar procedures to those described for Example 8C(isomers, dr=1:1). ¹H NMR (400 MHz, D₂O) δ 8.18 (s, 2H), 8.09 (s, 2H), 8.07 (s, 2H), 5.64-5.55 (m, 4H), 5.48 (s, 2H), 5.46-5.27 (m, 5H), 5.20-5.11 (m, 4H), 5.07-4.96 (m, 4H), 4.22-4.31 (m, 6H), 4.30-4.17 (m, 7H), 4.04-4.95 (m, 2H), 2.16 (s, 6H), 2.08 (s, 3H), 2.07 (s, 3H), 1.97 (s, 3H), 1.96 (s, 3H), 1.95 (s, 3H), 1.94 (s, 3H), 1.90 (s, 6H). ³¹P NMR (150 MHz, D₂O) δ 43.68, −15.13, −15.30. MS (ESI) m/z [M−H]⁻870.0.

### Example 127D

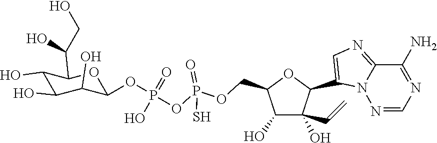 [SMILES: C=C[C@@]1(O)[C@H](O)[C@@H](COP(=O)(S)OP(=O)(O)OC2OC([C@@H](O)CO)C(O)C(O)C2O)O[C@H]1c1cnc2c(N)ncnn12]

The title compound was obtained from Example 127C with similar procedures to those described for Example 1D as TEA salt (1 eq.). ¹H NMR (400 MHz, D₂O) δ 8.19 (s, 1H), 8.08 (d, J=6.1 Hz, 1H), 5.62-5.26 (m, 4H), 5.08 (dd, J=10.4, 1.4 Hz, 1H), 4.57-4.26 (m, 4H), 4.15 (dd, J=8.7, 3.3 Hz, 1H), 3.99-3.90 (m, 1H), 3.86-3.61 (m, 4H), 3.40-3.33 (m, 1H), 3.20 (q, J=7.3 Hz, 6H), 1.28 (t, J=7.3 Hz, 9H). MS (ESI) m/z [M−H]⁻660.0.

### Example 128C

### Synthesis of Example 128C

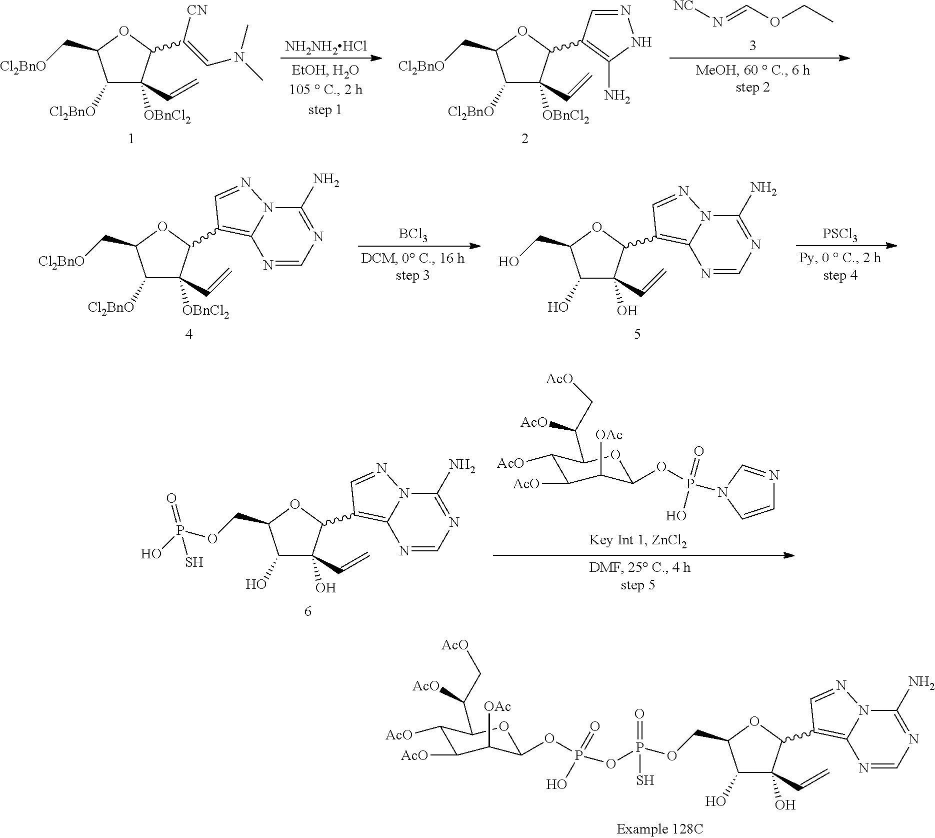

### Step 1: Synthesis of Compound 2

To a solution of compound 1 (10.0 g, 13.7 mmol) in EtOH (80 mL) and water (20 mL) was added hydrazine hydrochloride (4.68 g, 68.4 mmol). The reaction mixture was stirred for 2 h at 105° C., and then quenched by aq. NaHCO₃ (100.0 mL) at 20° C., extracted with EA (60 mL×3). The combined organic layers were washed with brine (120 mL), dried over Na₂SO₄, filtered and concentrated under reduced pressure. The residue was purified by column chromatography on silica gel (DCM/MeOH=10/1) to give the title compound 2 (6.40 g, 65.2% yield, 98.0% purity) as a white solid. ¹H NMR (400 MHz, CDCl₃) δ 7.53-7.35 (m, 3H), 7.35-7.28 (m, 3H), 7.27-7.12 (m, 4H), 5.97-5.67 (m, 1H), 5.47-5.25 (m, 2H), 5.11-4.88 (m, 1H), 4.84-4.57 (m, 6H), 4.46-4.28 (m, 1H), 3.92-3.72 (m, 2H). MS (ESI) m/z [M+H]⁺718.2.

### Step 2: Synthesis of Compound 4

To a solution of compound 2 (6.40 g, 8.91 mmol) in MeOH (64 mL) was added compound 3 (2.62 g, 26.73 mmol). The reaction mixture was stirred for 6 h at 60° C. and then concentrated under reduced pressure. The residue was purified by column chromatography on silica gel (PE/EA=1/1) to give the title compound 4 (1.30 g, 31.3% yield, 82.6% purity) as a white solid. ¹H NMR (400 MHz, CDCl₃) δ 8.18 (s, 1H), 8.13 (s, 1H), 7.67-7.28 (m, 6H), 7.27-7.14 (m, 3H), 6.61 (br s, 2H), 5.78 (dd, J=11.0, 17.6 Hz, 1H), 5.34-5.19 (m, 2H), 4.88-4.63 (m, 6H), 4.50-4.38 (m, 2H), 3.96-3.84 (m, 2H). MS (ESI) m/z [M+H]⁺770.2.

### Step 3: Synthesis of Compound 5

The title compound was obtained from compound 4 with similar procedures to those described for compound 12 in the synthesis of Example 125C(45 mg, 8.08% yield). ¹H NMR (400 MHz, D₂O) δ 8.14 (s, 1H), 8.03 (s, 1H), 5.57 (dd, J=10.8, 17.4 Hz, 1H), 5.32-5.20 (m, 2H), 5.07 (d, J=10.8 Hz, 1H), 4.26 (d, J=8.4 Hz, 1H), 4.11-3.97 (m, 2H), 3.88-3.82 (m, 1H), MS (ESI) m/z [M+H]⁺294.2.

### Step 4 and 5: Synthesis of Example 128C

The title compound was obtained with similar procedures to those described for Example 8C(isomers, dr=1:1). ¹H NMR (400 MHz, D₂O) δ 8.28 (s, 1H), 8.25 (s, 1H), 8.20 (s, 1H), 8.18 (s, 1H), 5.57-5.37 (m, 6H), 5.28-5.17 (m, 2H), 5.14-4.90 (m, 10H), 4.36-4.05 (m, 12H), 3.93-3.83 (m, 2H), 2.09 (s, 3H) 2.08 (s, 3H), 2.00 (s, 3H), 1.98 (s, 3H), 1.90 (s, 3H), 1.89 (s, 3H), 1.88 (s, 3H), 1.87 (s, 3H), 1.84 (s, 3H), 1.83 (s, 3H). ³¹P NMR (150 MHz, D₂O) δ 44.09, −15.14, −15.25. MS (ESI) m/z [M−H]⁻870.0.

### Example 142C

### Synthesis of Example 142C

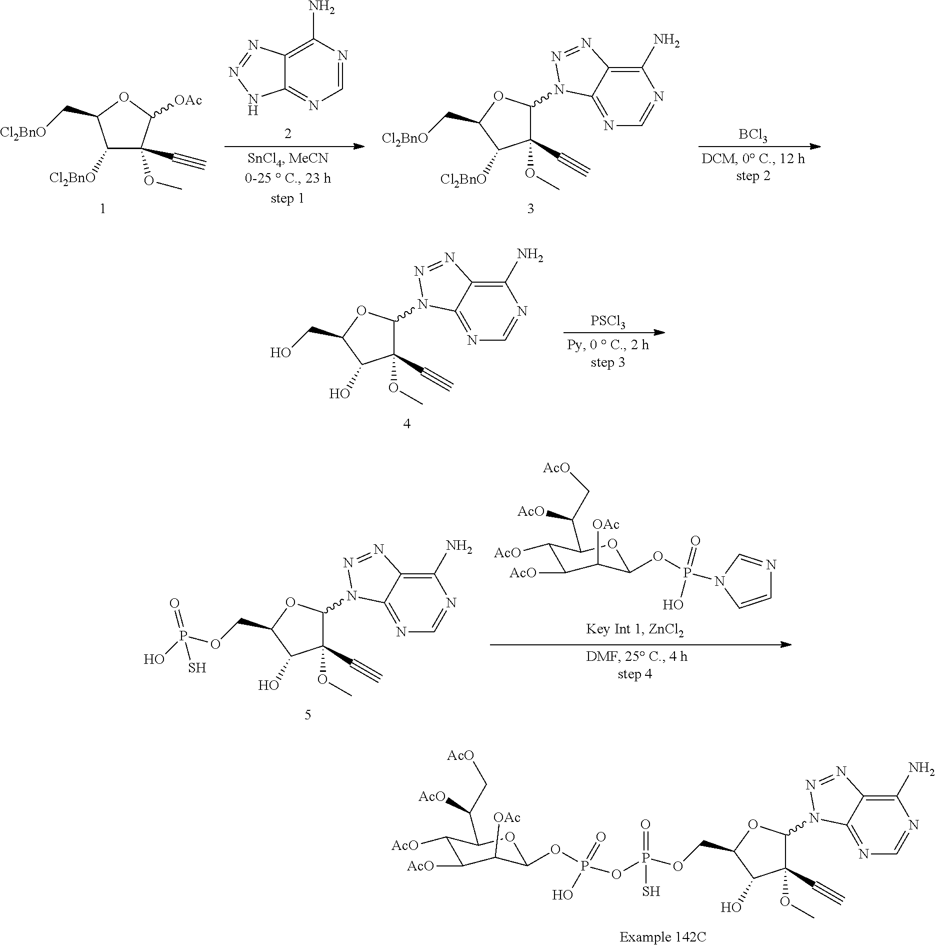

### Step 1: Synthesis of Compound 3

To a solution of compound 1 (2.00 g, 3.65 mmol) and compound 2 (497 mg, 3.65 mmol) in MeCN (50.0 mL) was added SnCl₄ (2.85 g, 10.9 mmol) at 0° C. The mixture was stirred at 25° C. for 23 h, then quenched with water (75 mL) and extracted with DCM (35 mL×3). The combined organic layers were washed with brine (15 mL), dried over Na₂SO₄, filtered and concentrated under reduced pressure. The residue was purified by prep-HPLC(column: Phenomenex luna C18, 250×70 mm #10 μm); mobile phase: [water (TFA)-ACN]; B %: 57%-87%, 20 min) to give the title compound 3 (500 mg, crude) as a white solid. ¹H NMR (400 MHz, CDCl₃) δ 8.46 (s, 1H), 7.47-7.41 (m, 2H), 7.39-7.36 (m, 1H), 7.36-7.32 (m, 1H), 7.24-7.19 (m, 2H), 6.64 (s, 1H), 6.17 (br s, 2H), 5.16 (d, J=9.2 Hz, 1H), 5.03 (d, J=12.4 Hz, 1H), 4.88-4.82 (m, 1H), 4.57 (s, 3H), 4.03-3.96 (m, 1H), 3.93-3.88 (m, 1H), 3.74 (s, 3H), 2.31 (s, 1H). MS (ESI) m/z [M+H]⁺625.1.

### Step 2: Synthesis of Compound 4

The title compound was obtained from compound 3 with similar procedures to those described for compound 12 in the synthesis of Example 125C(16.0 mg, 52.2 μmol, 7.25% yield, 100% purity). ¹H NMR (400 MHz, D₂O) δ 8.24 (s, 1H), 6.55 (s, 1H), 4.81 (d, J=9.2 Hz, 1H), 4.27-4.19 (m, 1H), 3.99-3.94 (m, 2H), 3.59 (s, 3H), 2.99 (s, 1H). MS (ESI) m/z [M+H]⁺307.1.

### Step 3 and 4: Synthesis of Example 142C

The title compound was obtained with similar procedures to those described for Example 8C. ¹H NMR (400 MHz, D₂O) δ 8.53 (s, 2H), 6.73 (s, 2H), 5.68-5.56 (m, 3H), 5.25-5.17 (m, 4H), 5.13-4.93 (m, 3H), 4.56-4.40 (m, 8H), 4.37-4.27 (m, 2H), 4.05 (d, J=10.0 Hz, 2H), 3.71 (s, 6H), 2.81 (s, 2H), 2.79 (s, 2H), 2.24 (s, 6H), 2.16 (s, 3H), 2.15 (s, 3H), 2.06 (s, 3H), 2.05 (s, 3H), 2.03 (s, 6H), 1.99 (s, 6H). MS (ESI) m/z [M−H]⁻883.0.

### Example 262C

### Synthesis of Example 262C

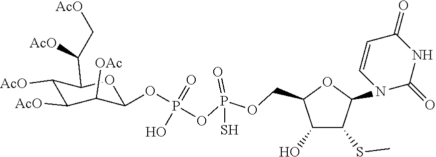 [SMILES: CS[C@@H]1[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1ccc(=O)[nH]c1=O]

The title compound was obtained with similar procedures to those described for Example 34C(26 mg, 31.3%) by using 1-beta-D-Arabinofuranosyluracil as the starting material and Sodium thiomethoxide instead of methylamine in step 3. ¹H NMR (400 MHz, D₂O) δ 8.00-7.95 (m, 1H), 6.09-6.07 (m, 1H), 5.97-5.93 (m, 1H), 5.64-5.48 (m, 2H), 5.28-5.19 (m, 2H), 5.10-5.02 (m, 1H), 4.52-4.47 (m, 1H), 4.44-4.34 (m, 1H), 4.31-4.11 (m, 4H), 4.10-4.04 (m, 1H), 3.54-3.45 (m, 1H), 2.16 (s, 3H), 2.11-2.04 (m, 6H), 2.02-1.94 (m, 6H), 1.91 (s, 3H). ³¹P NMR (162 MHz, D₂O) δ 43.85, −15.30. MS (ESI) m/z [M−H]⁻851.0.

### Example 262D

### Synthesis of Example 262D

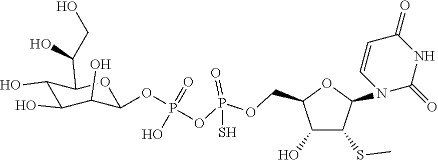 [SMILES: CS[C@@H]1[C@H](O)[C@@H](COP(=O)(S)OP(=O)(O)OC2OC([C@@H](O)CO)C(O)C(O)C2O)O[C@H]1n1ccc(=O)[nH]c1=O]

The title compound was obtained from Example 262C with similar procedures to those described for Example 1D as TEA salt (2.9 mg, 38.5%). ¹H NMR (400 MHz, D₂O) δ 8.00-7.95 (m, 1H), 6.10-6.08 (m, 1H), 5.95-5.93 (m, 1H), 5.24-5.17 (m, 2H), 4.56-4.51 (m, 1H), 4.28-4.23 (m, 1H), 4.21-4.12 (m, 2H), 4.07-4.02 (m, 1H), 3.90-3.84 (m, 1H), 3.77-3.58 (m, 4H), 3.56-3.48 (m, 1H), 3.35-3.29 (m, 1H), 3.11 (q, J=7.3 Hz, 6H), 2.06 (s, 3H), 1.19 (t, J=7.3 Hz, 9H). ³¹P NMR (162 MHz, D₂O) δ 43.12, −13.00. MS (ESI) m/z [M−H]⁻641.0.

### Example 263C

### Synthesis of Example 263C

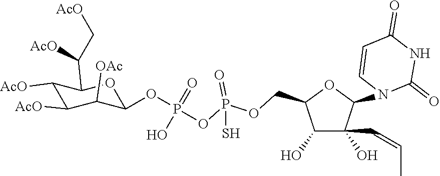 [SMILES: C/C=C\[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1ccc(=O)[nH]c1=O]

The title compound was obtained from relevant intermediate in preparing Example 96C with similar procedures to those described for Example 41C(9.3 mg, 13.7%). ¹H NMR (400 MHz, D₂O) δ 8.07-7.98 (m, 1H), 5.95-5.80 (m, 3H), 5.64-5.52 (m, 2H), 5.33-5.20 (m, 3H), 5.12-5.03 (m, 1H), 4.49-4.35 (m, 2H), 4.32-4.04 (m, 5H), 2.23-2.16 (m, 3H), 2.11-2.09 (m, 3H), 2.01 (s, 3H), 2.00-1.97 (m, 3H), 1.95-1.91 (m, 3H), 1.62-1.57 (m, 3H). ³¹P NMR (162 MHz, D₂O) δ 44.01, −14.96. MS (ESI) m/z [M−H]⁻861.1.

### Example 264C

### Synthesis of Example 264C

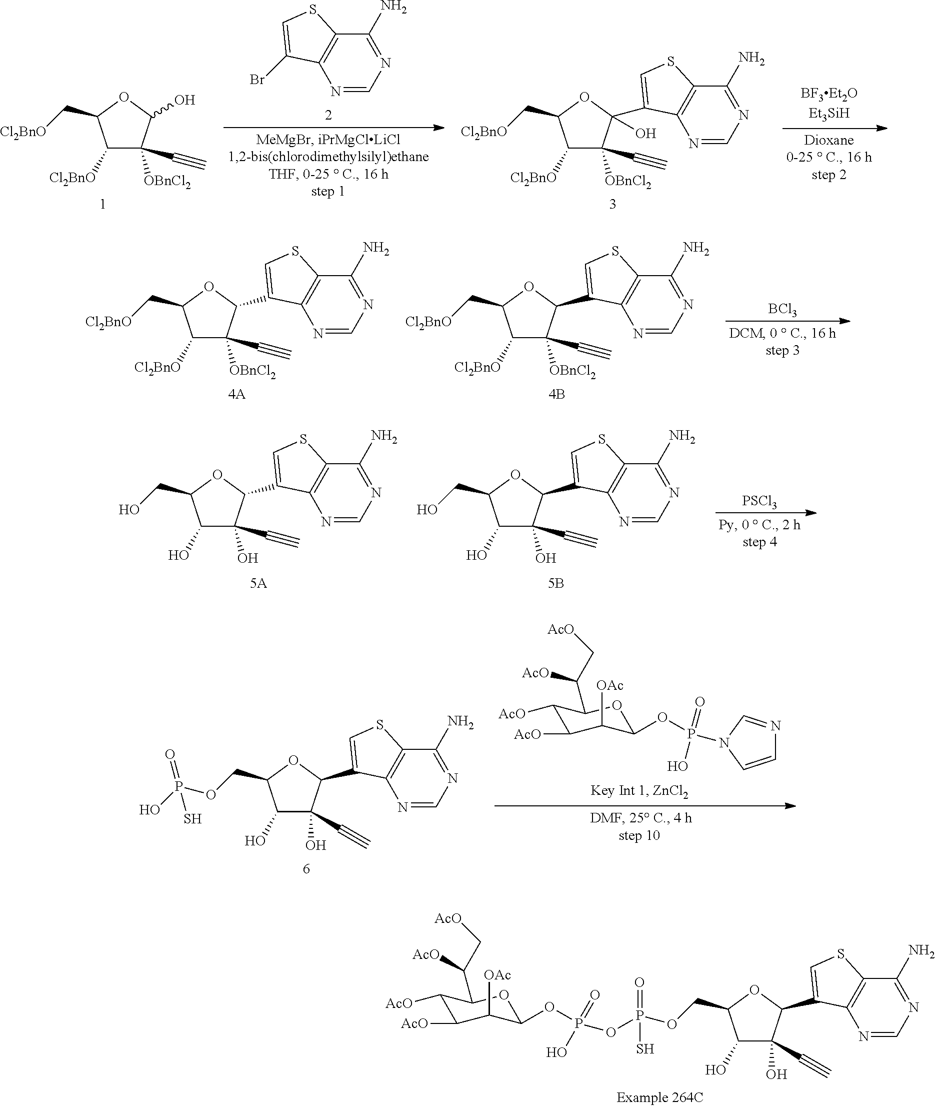

### Step 1: Synthesis of Compound 3

The title compound was obtained from compound 1 and 2 with similar procedures to those described for compound 4 in the synthesis of Example 127C(2.00 g, 7.77% yield). ¹H NMR (400 MHz, CDCl₃) δ 8.42 (s, 1H), 7.85 (s, 1H), 7.43-7.32 (m, 1H), 7.29-7.18 (m, 3H), 7.10-7.02 (m, 4H), 6.99-6.93 (m, 1H), 5.38-5.06 (m, 2H), 4.94-4.81 (m, 2H), 4.77-4.66 (m, 2H), 4.59-4.44 (m, 3H), 4.42-4.34 (m, 1H), 3.77-3.65 (m, 2H), 2.48 (s, 1H).

### Step 2: Synthesis of Compound 4A and 4B

The title compound was obtained from compound 3 with similar procedures to those described for compound 5 in the synthesis of Example 127C, which was carried out in DCM. Compound 4A (500 mg, 28.5% yield) and compound 4B (700 mg, 39.7% yield) were obtained as white solids.

Compound 4A: ¹H NMR (400 MHz, CDCl₃) δ 8.67 (s, 1H), 8.09 (s, 1H), 7.69-7.37 (m, 6H), 7.27-7.18 (m, 2H), 5.89 (s, 1H), 5.26-4.94 (m, 5H), 4.88-4.65 (m, 3H), 4.56-4.35 (m, 2H), 4.23-4.11 (m, 1H), 4.08-3.85 (m, 2H), 2.39 (s, 1H), 2.09 (d, J=1.8 Hz, 1H), 1.78-1.55 (m, 2H), 1.37-1.21 (m, 1H). MS (ESI) m/z [M+H]⁺784.0.

Compound 4B: ¹H NMR (400 MHz, CDCl₃) δ 8.67 (s, 1H), 8.09 (s, 1H), 7.69-7.37 (m, 6H), 7.27-7.18 (m, 2H), 5.89 (s, 1H), 5.26-4.94 (m, 5H), 4.88-4.65 (m, 3H), 4.56-4.35 (m, 2H), 4.23-4.11 (m, 1H), 4.08-3.85 (m, 2H), 2.39 (s, 1H), 2.09 (d, J=1.7 Hz, 1H), 1.78-1.55 (m, 2H), 1.37-1.21 (m, 1H). MS (ESI) m/z [M+H]⁺784.0.

### Step 3: Synthesis of Compound 5A and 5B

The title compound 5A and 5B were obtained respectively from compound 4A and 4B with similar procedures to those described for compound 12 in the synthesis of Example 125C. Compound 5A (100 mg, 51.1% yield) was obtained as a white solid and compound 5B (120 mg, 51.0% yield) was obtained as a white solid.

Compound 5A: ¹H NMR (400 MHz, D₂O) δ 8.24 (s, 1H), 8.11 (s, 1H), 5.54 (s, 1H), 4.66 (br s, 1H), 4.48 (br d, J=8.6 Hz, 1H), 4.10-3.98 (m, 1H), 3.82 (br d, J=2.2 Hz, 1H), 3.75-3.65 (m, 1H). MS (ESI) m/z [M+H]⁺308.1.

Compound 5B: ¹H NMR (400 MHz, DMSO-d₆) δ 8.37 (s, 1H), 8.02 (s, 1H), 7.56 (s, 2H), 6.81 (s, 1H), 5.37-5.35 (m, 1H), 5.25-5.15 (m, 1H), 5.05-4.95 (m, 1H), 4.11-4.09 (m, 1H), 3.90-3.80 (m, 1H), 3.75-3.50 (m, 2H), 3.05 (s, 1H). MS (ESI) m/z [M+H]⁺308.1.

### Step 4 and 5: Synthesis of Example 264C

The title compound was obtained from compound 5B with similar procedures to those described for Example 8C. ¹H NMR (400 MHz, D₂O) 88.75 (s, 1H), 8.74 (s, 1H), 8.51 (s, 1H), 8.48 (s, 1H), 5.72-5.42 (m, 5H), 5.40-5.22 (m, 6H), 5.19-5.08 (m, 2H), 4.54-4.23 (m, 11H), 4.21-4.05 (m, 2H), 2.81 (s, 1H), 2.80 (s, 1H), 2.25 (s, 6H), 2.14 (s, 6H), 2.06 (s, 3H), 2.05 (s, 3H), 2.04 (s, 3H), 2.02 (s, 3H), 2.00 (s, 3H), 1.99 (s, 3H). ³¹P NMR (150 MHz, D₂O) δ 43.97, −15.08. MS (ESI) m/z [M−H]⁻884.0.

### Example 265C

### Synthesis of Example 265C

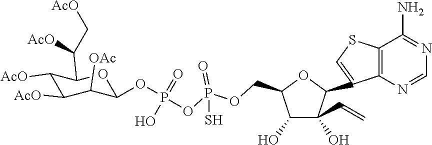 [SMILES: C=C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1csc2c(N)ncnc12]

The title compound was obtained with similar procedures to those described for Example 31C(7.2 mg, 12.9%) by using 7-bromothieno[3,2-d]pyrimidin-4-amine instead of 6-chloro-9H-purine. ¹H NMR (400 MHz, D₂O) δ 8.58-8.53 (m, 1H), 8.34-8.29 (m, 1H), 5.56-5.44 (m, 2H), 5.35-5.26 (m, 1H), 5.24-5.16 (m, 2H), 5.16-5.09 (m, 2H), 5.00-4.88 (m, 2H), 4.40-4.28 (m, 2H), 4.26-4.08 (m, 4H), 4.00-3.92 (m, 1H), 2.08 (s, 3H), 2.00-3.97 (m, 3H), 1.91-1.88 (m, 3H), 1.88-1.84 (m, 3H), 1.83 (s, 3H). ³¹P NMR (162 MHz, D₂O) δ 45.28, −14.12. MS (ESI) m/z [M−H]⁻886.0.

### Example 271C

### Synthesis of Example 271C

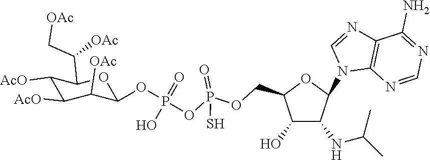 [SMILES: CC(=O)OC[C@H](OC(C)=O)C1OC(OP(=O)(O)OP(=O)(S)OC[C@H]2O[C@@H](n3cnc4c(N)ncnc43)[C@H](NC(C)C)[C@@H]2O)C(OC(C)=O)C(OC(C)=O)C1OC(C)=O]

The title compound was obtained with similar procedures to those described for Example 34C(60 mg, 37.5%) by using propan-2-amine instead of methylamine. ¹H NMR (400 MHz, D₂O) δ 8.72-8.67 (m, 1H), 8.35 (s, 1H), 6.53-6.47 (m, 1H), 5.61-5.51 (m, 2H), 5.26-5.12 (m, 2H), 5.08-5.00 (m, 1H), 4.88-4.83 (m, 1H), 4.69-4.60 (m, 1H), 4.50-4.45 (m, 1H), 4.42-4.34 (m, 1H), 4.30-4.12 (m, 3H), 4.08-3.99 (m, 1H), 3.51-3.42 (m, 1H), 2.17-2.13 (m, 3H), 2.08 (s, 3H), 1.99 (s, 3H), 1.97-1.93 (m, 3H), 1.92-1.89 (m, 3H), 1.27 (d, J=6.5 Hz, 3H), 1.20 (d, J=6.5 Hz, 3H). ³¹P NMR (162 MHz, D₂O) δ 43.76-43.32 (m, 1P), −15.21-−15.38 (m, 1P). MS (ESI) m/z [M−H]⁻885.2.

### Example 272C

### Synthesis of Example 272C

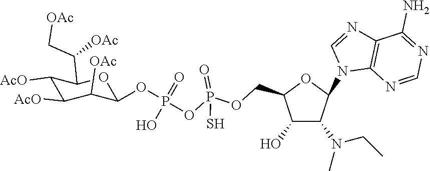 [SMILES: CCN(C)[C@@H]1[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1cnc2c(N)ncnc21]

The title compound was obtained with similar procedures to those described for Example 34C(56.4 mg, 45.6%). ¹H NMR (400 MHz, D₂O) δ 8.83-8.72 (m, 1H), 8.32 (s, 1H), 6.62-6.53 (m, 1H), 5.56-5.45 (m, 2H), 5.21-5.09 (m, 2H), 5.03-4.94 (m, 1H), 4.90-4.84 (m, 1H), 4.78-4.67 (m, 1H), 4.45-4.39 (m, 1H), 4.36-4.27 (m, 1H), 4.24-4.11 (m, 2H), 4.09-3.97 (m, 2H), 3.55-2.35 (m, 5H), 2.11-2.06 (m, 3H), 2.04-2.00 (m, 3H), 1.95-1.91 (m, 3H), 1.90-1.86 (m, 3H), 1.84 (s, 3H), 1.35-0.71 (m, 3H). ³¹P NMR (162 MHz, D₂O) δ 43.94-43.57 (m, 1P), −15.11-−15.28 (m, 1P). MS (ESI) m/z [M−H]⁻885.1.

### Example 321C

### Synthesis of Example 321C

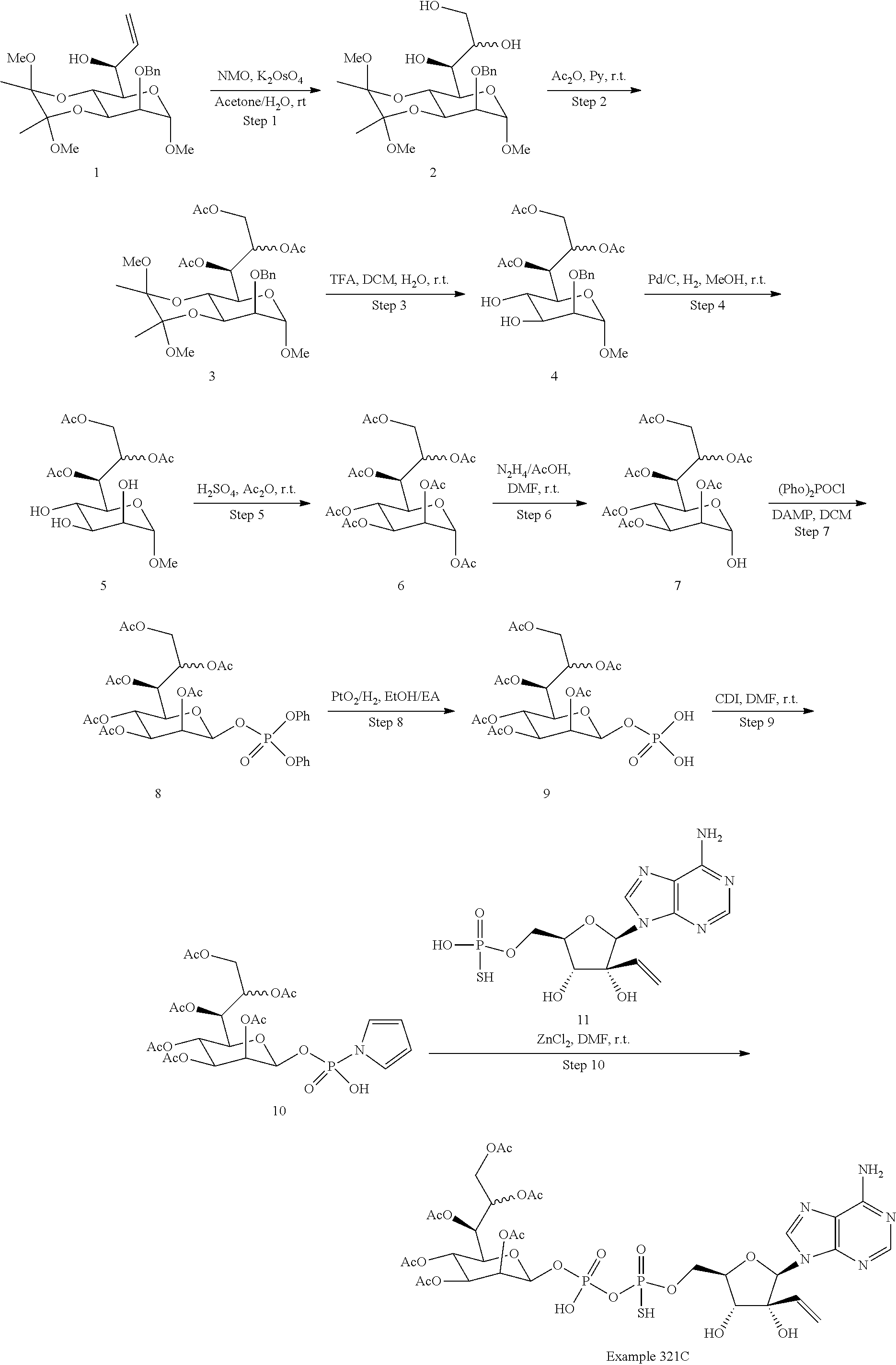

### Step 1: Synthesis of Compound 2

To a solution of(S)-1-((2S,3S,4aR,5R,7S,8S,8aS)-8-(benzyloxy)-2,3,7-trimethoxy-2,3-dimethylhexahydro-5H-pyrano[3,4-b][1,4]dioxin-5-yl) prop-2-en-1-ol (6 g, 11.66 mmol) in Acetone (20 mL) and H₂O 2O (20 mL) was added NMO (4.10 g, 34.98 mmol) and K₂OsO₄ (121.99 mg, 583.00 μmol). The reaction was stirred at 25° C. for 12 h, then saturated NaS₂O₃ solution (30 mL) was added to the mixture. The Acetone was removed in vacuum and the resulting aqueous mixture was extracted with EA. The organic layer was dried over anhydrous Na₂SO₄, filtered and concentrated under reduced pressure to afford the title compound (6.1 g, R/S-3/1, 95%) as a yellow oil. MS (ESI) m/z [M+Na]⁺481.1.

### Step 2: Synthesis of Compound 3

To a solution of compound 2 (7 g, 15.27 mmol) in Pyridine (20 mL) was added Ac₂O (6.23 g, 61.07 mmol) and DMAP (1.87 g, 15.27 mmol). The reaction was stirred at 25° C. for 12 h, then MeOH (5 mL) was added to the mixture and stirred for 20 min. The solution was concentrated and the residue was dispersed in water and EA, The organic layer was isolated and dried over anhydrous Na₂SO₄, filtered and concentrated under reduced pressure. The residue was purified with PE/EA=6/1 to afford the title compound (7.2 g, R/S=3/1, 80%) as a colorless oil. MS (ESI) m/z [M+Na]⁺607.1.

### Step 3: Synthesis of Compound 4

To a solution of compound 3 (1 g, 1.71 mmol) in DCM (10 mL) was added TFA (2.5 mL) and H₂O 2O (1 mL). The reaction was stirred at 25° C. for 12 h, then the mixture was concentrated under reduced pressure. The residue was dispersed in H₂O 2O and EA, the organic layer was isolated and dried over anhydrous Na₂SO₄, filtered and concentrated, the residue was purified with PE/EA=1/1 to afford the title compound (624 mg, R/S=3/1, 77%) as a yellow solid. MS (ESI) m/z [M+Na]⁺493.1.

### Step 4: Synthesis of Compound 5

To a solution of compound 4 (600 mg, 1.28 mmol) in MeOH (10 mL) was added Pd/C (20.43 mg, 192.00 μmol). The reaction was stirred at 25° C. under H₂ atmosphere for 12 h, then the mixture was filtered and the filtrate was concentrated under reduced pressure. The residue was purified with PE/EA=1/4 to afford the title compound (401 mg, R/S=3/1, 82%) as a colorless oil. MS (ESI) m/z [M+Na]⁺403.3.

### Step 5: Synthesis of Compound 6

To a solution of compound 5 (500 mg, 1.31 mmol) in Ac₂O (2.68 g, 26.29 mmol) was added H₂SO₄ (386.80 mg, 3.94 mmol). The reaction was stirred at 25° C. for 2 h, then the mixture was poured into ice water, and extracted with EA, the organic layer was concentrated under reduced pressure. The residue was purified with PE/EA=4/1 to afford the title compound (531 mg, R/S=3/1, 75%) as a colorless oil. MS (ESI) m/z [M+Na]⁺557.1.

### Step 6: Synthesis of Compound 7

To a solution of compound 6 (800 mg, 1.50 mmol) in DMF (5 mL) was added N2H₄AcOH (206.78 mg, 2.25 mmol). The reaction was stirred at 25° C. for 30 min, then the mixture was dispersed in EA and water, the organic layer was isolated and concentrated under reduced pressure. The residue was purified with PE/EA=4/1 to afford the title compound (700 mg, R/S=3/1, 94%) as a colorless oil. MS (ESI) m/z [M+Na]⁺515.2.

### Step 7: Synthesis of Compound 8

To a solution of compound 7 (700 mg, 1.42 mmol) in DCM (10 mL) was added DMAP (347.33 mg, 2.84 mmol) and then a solution of diphenyl chlorophosphite (763.74 mg, 2.84 mmol) in DCM (5 mL) was added slowly. The reaction was stirred at 25° C. for 12 h, then the mixture was dispersed in DCM and water, the organic layer was isolated and concentrated under reduced pressure. The residue was purified with PE/EA (4/1) afforded the title compound (1 g, R/S=3/1, 87%) as a colorless oil. MS (ESI) m/z [M+Na]⁺747.1.

### Step 8: Synthesis of Compound 9

To a solution of compound 8 (450 mg, 621.03 μmol) in EA (5 mL) and EtOH (5 mL) was added PtO₂ (28.20 mg, 124.21 μmol). The reaction was stirred at 25° C. under H₂ atmosphere for 24 h, then the mixture was filtered and concentrated under reduced pressure to afford the title compound (267 mg, R/S=3/1, 75% yield) as a white solid. MS (ESI) m/z [M+H]⁺573.1

### Step 9 and 10: Synthesis of Example 321C

The title compound was obtained with similar procedures to those described for Example 8C(60 mg, 39.6% for two steps). ¹H NMR (400 MHz, D₂O) δ 8.71-8.47 (m, 1H), 8.24 (s, 1H), 6.06-5.97 (m, 1H), 5.60-5.47 (m, 2H), 5.32-5.07 (m, 5H), 5.00-4.84 (m, 2H), 4.53-4.44 (m, 1H), 4.43-4.30 (m, 2H), 4.27-4.16 (m, 3H), 4.12-4.03 (m, 1H), 2.11-2.07 (m, 3H), 2.05-2.01 (m, 3H), 1.99-1.95 (m, 3H), 1.95-1.89 (m, 6H), 1.84-1.81 (m, 3H). ³¹P NMR (162 MHz, D₂O) δ 43.73, −15.46. MS (ESI) m/z [M−H]⁻870.1.

### Example 25A

### Synthesis of Example 25A

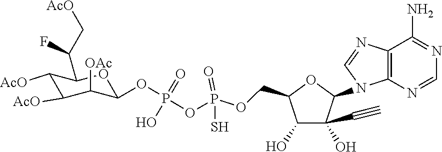 [SMILES: C#C[C@@]1(O)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1cnc2c(N)ncnc21]

The title compound was obtained from key Int 2 with similar procedures to those described for Example 25C(48 mg, 32%). ¹H NMR (400 MHz, D₂O) δ 8.72-8.70 (m, 1H), 8.44 (s, 1H), 6.24 (s, 1H), 5.68-5.64 (m, 2H), 5.37-5.28 (m, 2H), 4.93-4.91 (m, 1H), 4.68-4.54 (m, 1H), 4.54-4.22 (m, 5H), 4.10-4.01 (m, 1H), 2.66-2.65 (m, 1H), 2.22 (s, 3H), 2.14-2.06 (m, 6H), 1.99 (s, 3H). ³¹P NMR (162 MHz, D₂O) δ 44.19, −15.23. ¹⁹F NMR (376 MHz, D₂O) δ−206.37, −206.40. MS (ESI) m/z [M−H]⁻827.8.

### Example 25B

### Synthesis of Example 25B

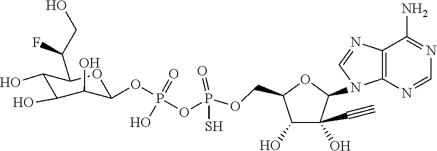 [SMILES: C#C[C@@]1(O)[C@H](O)[C@@H](COP(=O)(S)OP(=O)(O)OC2OC([C@@H](F)CO)C(O)C(O)C2O)O[C@H]1n1cnc2c(N)ncnc21]

The title compound was obtained from Example 25A with similar procedures to those described for Example 1D (48 mg, 32%). ¹H NMR (400 MHz, D₂O) δ 8.67-8.59 (m, 1H), 8.25 (s, 1H), 6.25 (s, 1H), 5.28-5.19 (m, 1H), 4.64-4.58 (m, 1H), 4.48-4.38 (m, 1H), 4.36-4.24 (m, 2H), 4.11-4.05 (m, 1H), 3.97-3.86 (m, 1H), 3.85-3.71 (m, 2H), 3.65-3.55 (m, 1H), 3.45-3.27 (m, 1H), 2.58 (s, 1H). ³¹P NMR (162 MHz, D₂O) δ 43.67, −14.58. ¹⁹F NMR (376 MHz, D₂O) δ −208.48, −208.51. MS (ESI) m/z [M−H]⁻659.8.

### Example 33A

### Synthesis of Example 33A

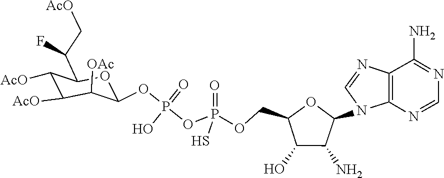 [SMILES: CC(=O)OC[C@H](F)C1OC(OP(=O)(O)OP(=O)(S)OC[C@H]2O[C@@H](n3cnc4c(N)ncnc43)[C@H](N)[C@@H]2O)C(OC(C)=O)C(OC(C)=O)C1OC(C)=O]

Two isomers of the title compound were obtained from Example 59A with similar procedures to those described for Example 33C.

Isomer 1 (2 mg, 10.3%): ¹H NMR (400 MHz, D₂O) δ 8.71 (s, 1H), 8.38 (s, 1H), 6.48 (d, J=7.3 Hz, 1H), 5.59 (d, J=9.6 Hz, 1H), 5.56 (d, J=2.7 Hz, 1H), 5.31-5.22 (m, 2H), 4.87 (t, J=5.5 Hz, 1H), 4.81-4.79 (m, 1H), 4.63-4.58 (m, 1H), 4.51-4.48 (m, 1H), 4.41 (d, J=5.6 Hz, 1H), 4.39-4.30 (m, 1H), 4.26-4.12 (m, 2H), 4.03-3.94 (m, 1H), 2.16 (s, 3H), 2.05 (s, 3H), 2.02 (s, 3H), 1.95 (s, 3H). ³¹P NMR (162 MHz, D₂O) δ 43.76, −15.32. ¹⁹F NMR (376 MHz, D₂O) δ-206.40. MS (ESI) m/z [M−H]⁻802.8.

Isomer 2 (3 mg, 15.5%): ¹H NMR (400 MHz, D₂O) δ 8.55 (s, 1H), 8.24 (s, 1H), 6.40 (d, J=7.2 Hz, 1H), 5.56 (d, J=2.8 Hz, 1H), 5.52 (d, J=9.6 Hz, 1H), 5.29-5.21 (m, 1H), 5.18-5.15 (m, 1H), 4.83-4.79 (m, 1H), 4.75-4.73 (m, 1H), 4.53-4.49 (m, 1H), 4.44-4.41 (m, 1H), 4.39-4.28 (m, 2H), 4.20-4.08 (m, 2H), 3.96-3.87 (m, 1H), 2.10 (s, 3H), 2.01 (s, 3H), 1.99 (s, 3H), 1.89 (s, 3H). ³¹P NMR (162 MHz, D₂O) δ 43.48, −15.32. ¹⁹F NMR (376 MHz, D₂O) δ-206.32. MS (ESI) m/z [M−H]⁻802.8.

### Biological Assays

### Biological Example 1. The Compounds Disclosed Herein are Potent Agonists of ALPK1.

The compounds disclosed herein activate NF-κB through ALPK1-TIFA axis as determined by the NF-κB luciferase reporter and TIFA phosphorylation. For NF-κB luciferase assay, the plasmids (pNL2.2-BII-5RE-Luc), and control vector (pRL-TK) were transfected into 293T cells with the Jetprime reagents (Polyplus). NF-κB luciferase activity was determined using the dual luciferase assay kit (Promega) according to the manufacturer's instructions. TIFA phosphorylation upon the treatment with the compounds disclosed herein described in Table 1, were assayed with immunoblotting. Briefly, for the immunoblotting assay, the 293T cells were seeded in 12-well-plates and cultured for 16 h, and treated with indicated compounds for another 2 hours. Subsequently, the cells were collected and sampled as the regular western-blotting method, immunoblotted by the TIFA phosphorylation specific antibody (ab214815, Abcam).

The EC₅₀ of the compounds disclosed herein were determined by the NF-κB luciferase reporter assay using 293T cells described supra. Compounds with indicated concentrations were added to the culture medium of 293T and the NF-κB luciferase activity was determined using the dual luciferase assay kit (Promega) according to the manufacturer's instructions. The results indicate that compounds provided herein exhibit activity for the activation of ALPK1. Thus, in light of the foregoing, chemical entities of this disclosure exhibit agonistic activity against ALPK1. These chemical entities induce strong activation of the immune regulator NF-κB.

### Biological Example 2. Assessment of PK/Tissue Distribution in Sprague-Dawley Rats and Mice.

Tissue distribution study of the compounds disclosed herein were conducted in SD rats and mice to assess their liver-targeting properties. The compounds disclosed herein were orally administered to SD rats and mice, and the concentration of the compound and its metabolites were tested in plasma and liver at different time points.

### Rat PK Studies

The pharmacokinetics of the compounds disclosed herein were evaluated in female Sprague-Dawley rats after administration via oral gavage and intravenous bolus (IV) injection. The test compound was dissolved in 0.5% methylcellulose for oral gavage and in 30% HP-β-CD for IV injection. The animals used for oral administration were fasted overnight prior to dosing and fed normal daily feed at 4 hours post-dose, but the animals used for IV dosing had free access to food during the study. Plasma samples were collected at pre-dose, 0.033 (IV only), 0.083, 0.25, 0.5, 1, 2, 4, 7 and 24 hours post-dose. Liver samples were collected at 1, 3 and 6 hours post-dose. The samples were analyzed by LC/MS/MS and the concentrations of test compound and its metabolites at each time point were determined. Pharmacokinetic parameters were calculated from the plasma concentrations using Pheonix WinNonlin.

Mouse PK studies

The pharmacokinetics of the compounds disclosed herein were evaluated in female C57BL/6 mice after administration via oral gavage, intravenous bolus (IV) injection, intraperitoneal (IP) injection and subcutaneous (SC) injection. The test compound was dissolved in 0.5% methylcellulose for oral gavage and in 30% HP-β-CD for IV, IP and SC injections. All animals had free access to food during the whole study. Plasma samples were collected at pre-dose, 0.033 (IV only), 0.083, 0.25, 0.5, 1, 2, 4, 7, 24 and 48 hours post-dose. Liver samples were collected at 1, 3 and 6 hours post-dose. Samples were analyzed by LC/MS/MS and the concentrations of test compound and its metabolites at each time point were determined. Pharmacokinetic parameters were calculated from the plasma concentrations using Pheonix WinNonlin.

The result shows that the compounds disclosed herein showed higher concentration in liver than in plasma, especially those with vinyl group or amine group, and those with both vinyl and hydroxyl groups. Particularly, preferable compounds have a ratio for liver concentration to plasma concentration of greater than 2, more preferable compounds have a ratio for liver concentration to plasma concentration of greater than 5, and most preferable compounds have a ratio for liver concentration to plasma concentration of greater than 10. The PK properties and tissue exposure of representative compounds and control compound ADPS-Heptose in Rats are given below.

| | PK properties in Rats | | Tissue Exposure in Rats | | |
|---|---|---|---|---|---|
| | T_{1/2} post PO | CL post IV | | Cₘₐₓ in | Cₘₐₓ in |
| | Dosing | Dosing | Dose, | Plasma, | Liver, |
| Compound | @5 mg/kg, h | @1 mg/kg, mL/min/kg | mg/kg | ng/ml | ng/g |
| Example 31C | 1.73 | 14.6 | PO, 5 | <1 | 30.5 |
| Example 35C | 0.45 | 7.67 | PO, 5 | 75.5 | 1331 |
| Example 41C | 0.85 | 9.76 | PO, 5 | <1 | 18.7 |
| Example 42C | 0.63 | 18.6 | PO, 5 | 2.4 | 31.7 |
| Example 43C | 2.34 | 8.25 | PO, 5 | <1 | 22.4 |
| Example 127C | 0.64 | 17.6 | PO, 5 | 0.4 | 48.5 |
| ADPS-Heptose | 0.72 | 42.9 | PO, 5 | <1 | 2.0 |
| 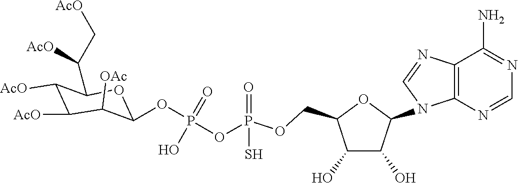 [SMILES: CC(=O)OC[C@H](OC(C)=O)C1OC(OP(=O)(O)OP(=O)(S)OC[C@H]2O[C@@H](n3cnc4c(N)ncnc43)[C@H](O)[C@@H]2O)C(OC(C)=O)C(OC(C)=O)C1OC(C)=O] | | | | | |

TABLE 1
Representative compounds of the present disclosure

| Examples | Structures |
|---|---|
| Example 21A | 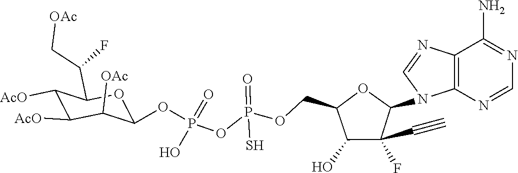 [SMILES: C#C[C@@]1(F)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1cnc2c(N)ncnc21] |
| Example 21B | 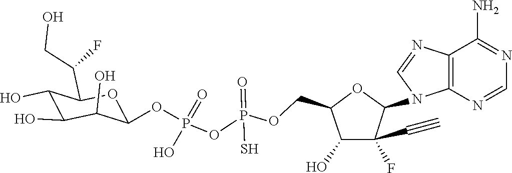 [SMILES: C#C[C@@]1(F)[C@H](O)[C@@H](COP(=O)(S)OP(=O)(O)OC2OC([C@@H](F)CO)C(O)C(O)C2O)O[C@H]1n1cnc2c(N)ncnc21] |
| Example 21C | 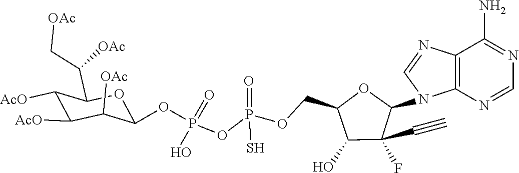 [SMILES: C#C[C@@]1(F)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1cnc2c(N)ncnc21] |
| Example 21D | 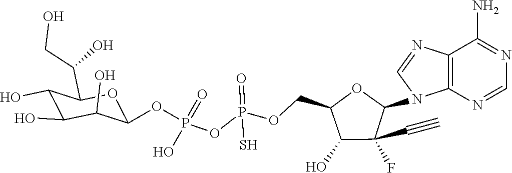 [SMILES: C#C[C@@]1(F)[C@H](O)[C@@H](COP(=O)(S)OP(=O)(O)OC2OC([C@@H](O)CO)C(O)C(O)C2O)O[C@H]1n1cnc2c(N)ncnc21] |
| Example 22A | 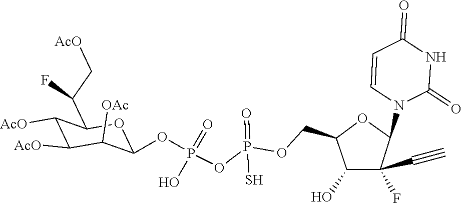 [SMILES: C#C[C@@]1(F)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1ccc(=O)[nH]c1=O] |
| Example 22B | 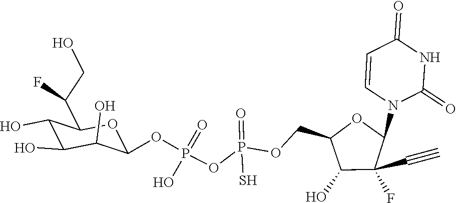 [SMILES: C#C[C@@]1(F)[C@H](O)[C@@H](COP(=O)(S)OP(=O)(O)OC2OC([C@@H](F)CO)C(O)C(O)C2O)O[C@H]1n1ccc(=O)[nH]c1=O] |
| Example 22C | 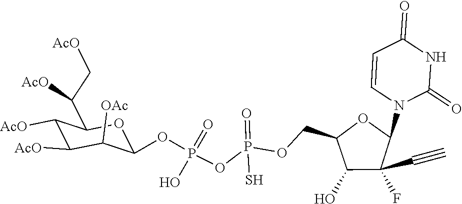 [SMILES: C#C[C@@]1(F)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1ccc(=O)[nH]c1=O] |
| Example 22D | 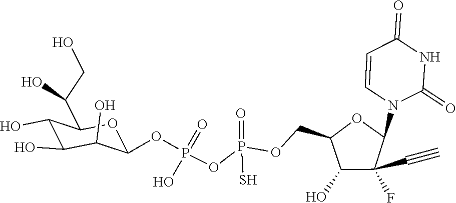 [SMILES: C#C[C@@]1(F)[C@H](O)[C@@H](COP(=O)(S)OP(=O)(O)OC2OC([C@@H](O)CO)C(O)C(O)C2O)O[C@H]1n1ccc(=O)[nH]c1=O] |
| Example 25A | 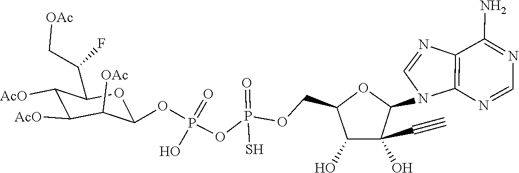 [SMILES: C#C[C@@]1(O)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1cnc2c(N)ncnc21] |
| Example 25B | 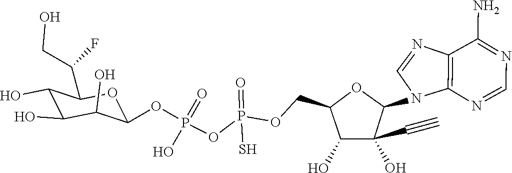 [SMILES: C#C[C@@]1(O)[C@H](O)[C@@H](COP(=O)(S)OP(=O)(O)OC2OC([C@@H](F)CO)C(O)C(O)C2O)O[C@H]1n1cnc2c(N)ncnc21] |
| Example 25C | 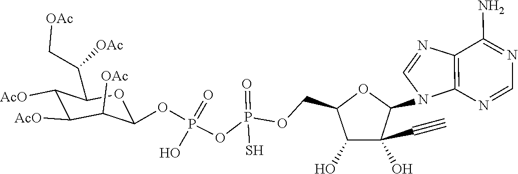 [SMILES: C#C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1cnc2c(N)ncnc21] |
| Example 25D | 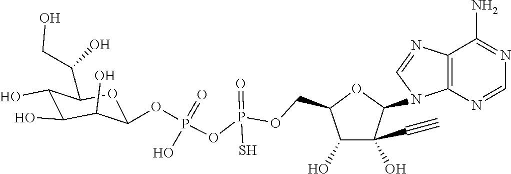 [SMILES: C#C[C@@]1(O)[C@H](O)[C@@H](COP(=O)(S)OP(=O)(O)OC2OC([C@@H](O)CO)C(O)C(O)C2O)O[C@H]1n1cnc2c(N)ncnc21] |
| Example 26A | 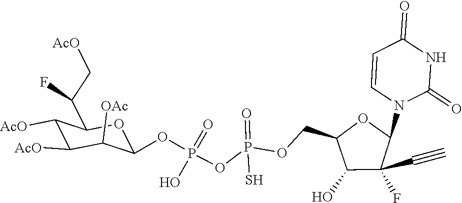 [SMILES: C#C[C@@]1(F)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1ccc(=O)[nH]c1=O] |
| Example 26B | 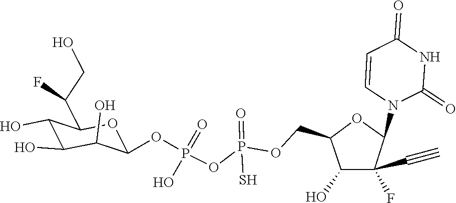 [SMILES: C#C[C@@]1(F)[C@H](O)[C@@H](COP(=O)(S)OP(=O)(O)OC2OC([C@@H](F)CO)C(O)C(O)C2O)O[C@H]1n1ccc(=O)[nH]c1=O] |
| Example 26C | 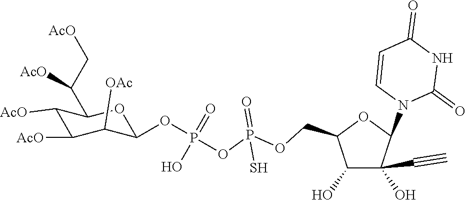 [SMILES: C#C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1ccc(=O)[nH]c1=O] |
| Example 26D | 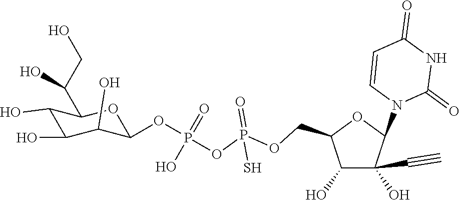 [SMILES: C#C[C@@]1(O)[C@H](O)[C@@H](COP(=O)(S)OP(=O)(O)OC2OC([C@@H](O)CO)C(O)C(O)C2O)O[C@H]1n1ccc(=O)[nH]c1=O] |
| Example 26E | 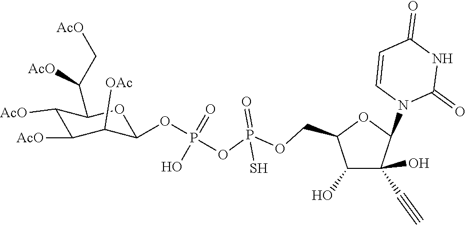 [SMILES: C#C[C@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1ccc(=O)[nH]c1=O] |
| Example 31A | 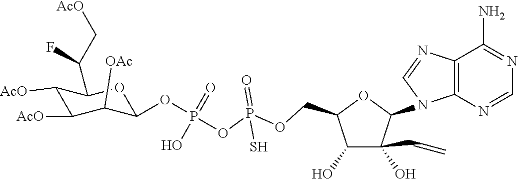 [SMILES: C=C[C@@]1(O)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1cnc2c(N)ncnc21] |
| Example 31B | 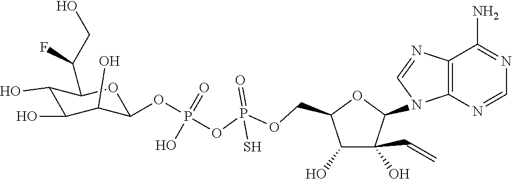 [SMILES: C=C[C@@]1(O)[C@H](O)[C@@H](COP(=O)(S)OP(=O)(O)OC2OC([C@@H](F)CO)C(O)C(O)C2O)O[C@H]1n1cnc2c(N)ncnc21] |
| Example 31C | 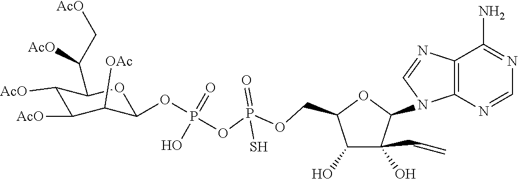 [SMILES: C=C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1cnc2c(N)ncnc21] |
| Example 31D | 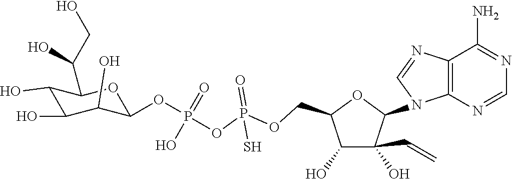 [SMILES: C=C[C@@]1(O)[C@H](O)[C@@H](COP(=O)(S)OP(=O)(O)OC2OC([C@@H](O)CO)C(O)C(O)C2O)O[C@H]1n1cnc2c(N)ncnc21] |
| Example 31E | 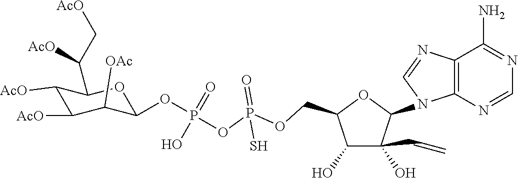 [SMILES: C=C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1cnc2c(N)ncnc21] |
| Example 31F | 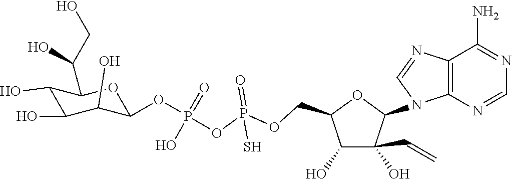 [SMILES: C=C[C@@]1(O)[C@H](O)[C@@H](COP(=O)(S)OP(=O)(O)OC2OC([C@@H](O)CO)C(O)C(O)C2O)O[C@H]1n1cnc2c(N)ncnc21] |
| Example 31G | 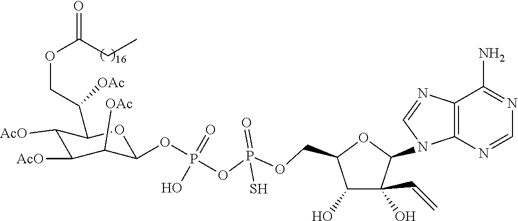 [SMILES: C=C[C@@]1(O)[C@H](O)[C@@H](COP(=O)(S)OP(=O)(O)OC2OC([C@H](COC(=O)CC)OC(C)=O)C(OC(C)=O)C(OC(C)=O)C2OC(C)=O)O[C@H]1n1cnc2c(N)ncnc21] |
| Example 31H | 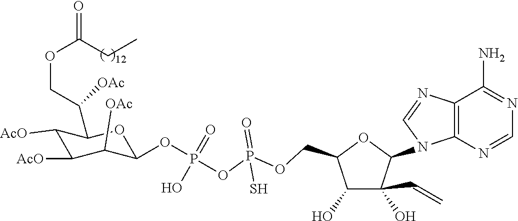 [SMILES: C=C[C@@]1(O)[C@H](O)[C@@H](COP(=O)(S)OP(=O)(O)OC2OC([C@H](COC(=O)CC)OC(C)=O)C(OC(C)=O)C(OC(C)=O)C2OC(C)=O)O[C@H]1n1cnc2c(N)ncnc21] |
| Example 31I | 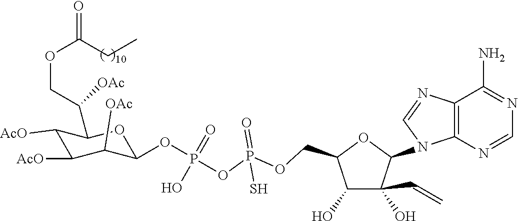 [SMILES: C=C[C@@]1(O)[C@H](O)[C@@H](COP(=O)(S)OP(=O)(O)OC2OC([C@H](COC(=O)CC)OC(C)=O)C(OC(C)=O)C(OC(C)=O)C2OC(C)=O)O[C@H]1n1cnc2c(N)ncnc21] |
| Example 31J | 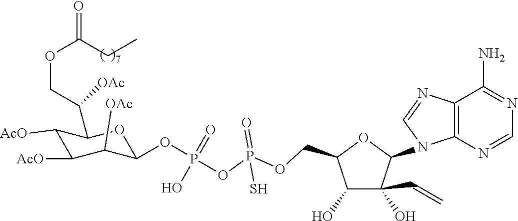 [SMILES: C=C[C@@]1(O)[C@H](O)[C@@H](COP(=O)(S)OP(=O)(O)OC2OC([C@H](COC(=O)CC)OC(C)=O)C(OC(C)=O)C(OC(C)=O)C2OC(C)=O)O[C@H]1n1cnc2c(N)ncnc21] |
| Example 31K | 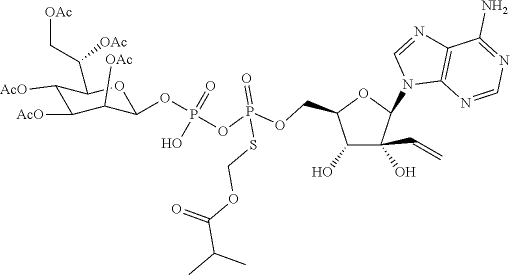 [SMILES: C=C[C@@]1(O)[C@H](O)[C@@H](COP(=O)(OP(=O)(O)OC2OC([C@H](COC(C)=O)OC(C)=O)C(OC(C)=O)C(OC(C)=O)C2OC(C)=O)SCOC(=O)C(C)C)O[C@H]1n1cnc2c(N)ncnc21] |
| Example 31L | 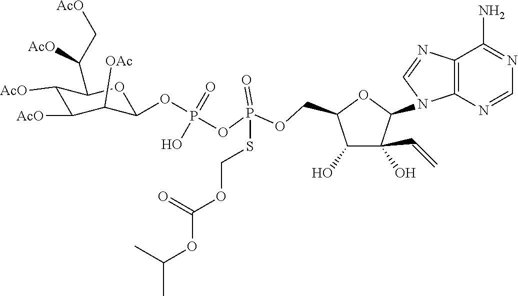 [SMILES: C=C[C@@]1(O)[C@H](O)[C@@H](COP(=O)(OP(=O)(O)OC2OC([C@H](COC(C)=O)OC(C)=O)C(OC(C)=O)C(OC(C)=O)C2OC(C)=O)SCOC(=O)OC(C)C)O[C@H]1n1cnc2c(N)ncnc21] |
| Example 31M | 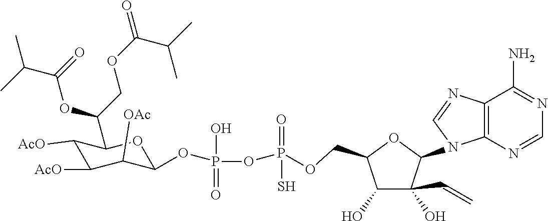 [SMILES: C=C[C@@]1(O)[C@H](O)[C@@H](COP(=O)(S)OP(=O)(O)OC2OC([C@H](COC(=O)C(C)C)OC(=O)C(C)C)C(OC(C)=O)C(OC(C)=O)C2OC(C)=O)O[C@H]1n1cnc2c(N)ncnc21] |
| Example 31O | 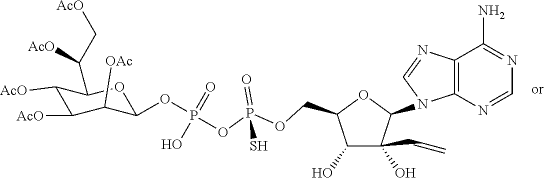 [SMILES: C=C[C@@]1(O)[C@H](O)[C@@H](CO[P@](=O)(S)OP(=O)(O)OC2OC([C@H](COC(C)=O)OC(C)=O)C(OC(C)=O)C(OC(C)=O)C2OC(C)=O)O[C@H]1n1cnc2c(N)ncnc21] |
| | 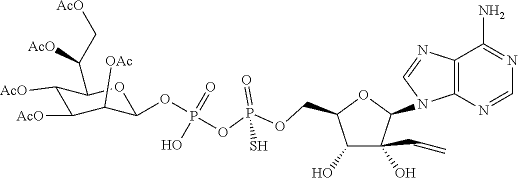 [SMILES: C=C[C@@]1(O)[C@H](O)[C@@H](CO[P@@](=O)(S)OP(=O)(O)OC2OC([C@H](COC(C)=O)OC(C)=O)C(OC(C)=O)C(OC(C)=O)C2OC(C)=O)O[C@H]1n1cnc2c(N)ncnc21] |
| Example 31P | 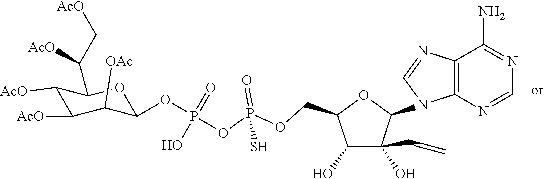 [SMILES: C=C[C@@]1(O)[C@H](O)[C@@H](CO[P@@](=O)(S)OP(=O)(O)OC2OC([C@H](COC(C)=O)OC(C)=O)C(OC(C)=O)C(OC(C)=O)C2OC(C)=O)O[C@H]1n1cnc2c(N)ncnc21] |
| | 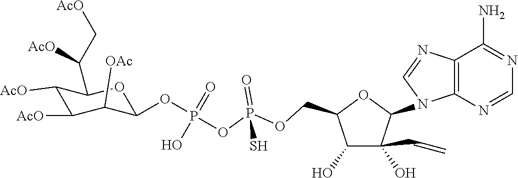 [SMILES: C=C[C@@]1(O)[C@H](O)[C@@H](CO[P@](=O)(S)OP(=O)(O)OC2OC([C@H](COC(C)=O)OC(C)=O)C(OC(C)=O)C(OC(C)=O)C2OC(C)=O)O[C@H]1n1cnc2c(N)ncnc21] |
| Example 31Q | 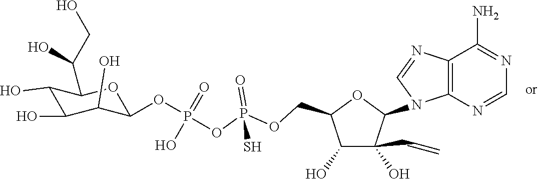 [SMILES: C=C[C@@]1(O)[C@H](O)[C@@H](CO[P@](=O)(S)OP(=O)(O)OC2OC([C@@H](O)CO)C(O)C(O)C2O)O[C@H]1n1cnc2c(N)ncnc21] |
| | 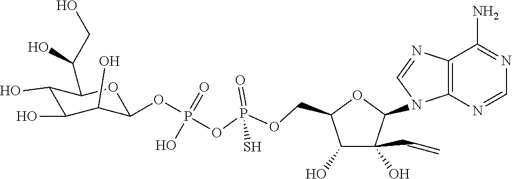 [SMILES: C=C[C@@]1(O)[C@H](O)[C@@H](CO[P@@](=O)(S)OP(=O)(O)OC2OC([C@@H](O)CO)C(O)C(O)C2O)O[C@H]1n1cnc2c(N)ncnc21] |
| Example 31R | 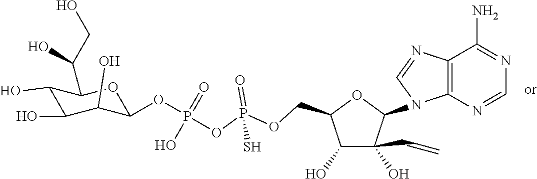 [SMILES: C=C[C@@]1(O)[C@H](O)[C@@H](CO[P@@](=O)(S)OP(=O)(O)OC2OC([C@@H](O)CO)C(O)C(O)C2O)O[C@H]1n1cnc2c(N)ncnc21] |
| | 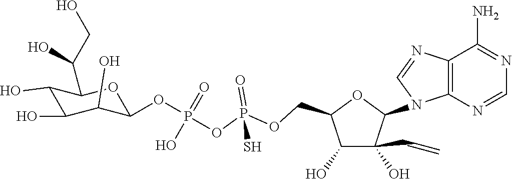 [SMILES: C=C[C@@]1(O)[C@H](O)[C@@H](CO[P@](=O)(S)OP(=O)(O)OC2OC([C@@H](O)CO)C(O)C(O)C2O)O[C@H]1n1cnc2c(N)ncnc21] |
| Example 33A | 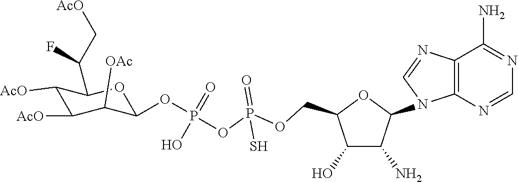 [SMILES: CC(=O)OC[C@H](F)C1OC(OP(=O)(O)OP(=O)(S)OC[C@H]2O[C@@H](n3cnc4c(N)ncnc43)[C@H](N)[C@@H]2O)C(OC(C)=O)C(OC(C)=O)C1OC(C)=O] |
| Example 33B | 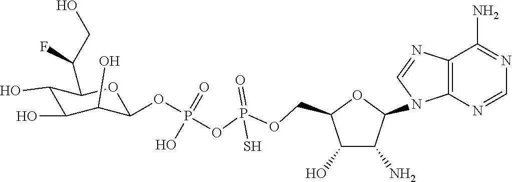 [SMILES: Nc1ncnc2c1ncn2[C@@H]1O[C@H](COP(=O)(S)OP(=O)(O)OC2OC([C@@H](F)CO)C(O)C(O)C2O)[C@@H](O)[C@H]1N] |
| Example 33C | 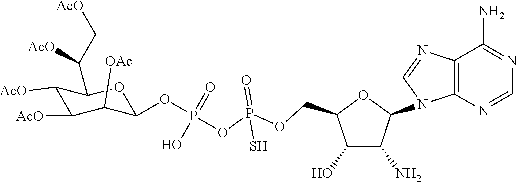 [SMILES: CC(=O)OC[C@H](OC(C)=O)C1OC(OP(=O)(O)OP(=O)(S)OC[C@H]2O[C@@H](n3cnc4c(N)ncnc43)[C@H](N)[C@@H]2O)C(OC(C)=O)C(OC(C)=O)C1OC(C)=O] |
| Example 33D | 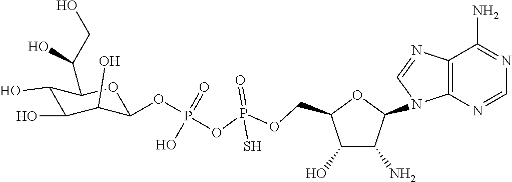 [SMILES: Nc1ncnc2c1ncn2[C@@H]1O[C@H](COP(=O)(S)OP(=O)(O)OC2OC([C@@H](O)CO)C(O)C(O)C2O)[C@@H](O)[C@H]1N] |
| Example 33E | 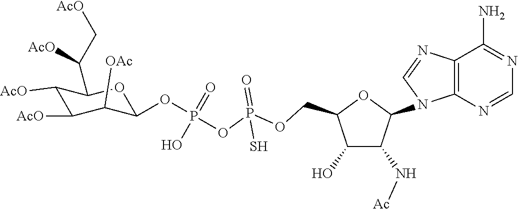 [SMILES: CC(=O)N[C@@H]1[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1cnc2c(N)ncnc21] |
| Example 34A | 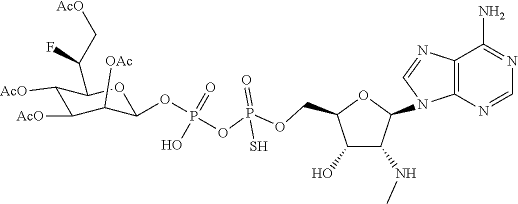 [SMILES: CN[C@@H]1[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1cnc2c(N)ncnc21] |
| Example 34B | 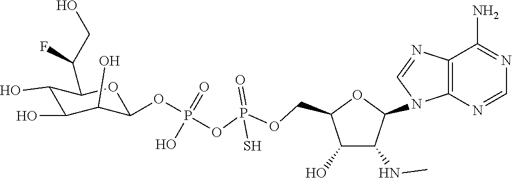 [SMILES: CN[C@@H]1[C@H](O)[C@@H](COP(=O)(S)OP(=O)(O)OC2OC([C@@H](F)CO)C(O)C(O)C2O)O[C@H]1n1cnc2c(N)ncnc21] |
| Example 34C | 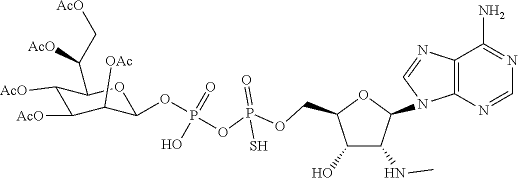 [SMILES: CN[C@@H]1[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1cnc2c(N)ncnc21] |
| Example 34D | 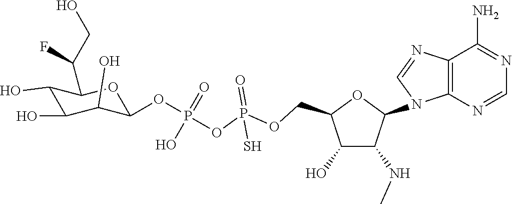 [SMILES: CN[C@@H]1[C@H](O)[C@@H](COP(=O)(S)OP(=O)(O)OC2OC([C@@H](F)CO)C(O)C(O)C2O)O[C@H]1n1cnc2c(N)ncnc21] |
| Example 35A | 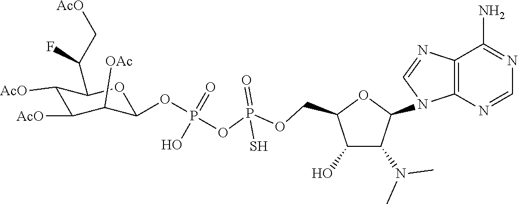 [SMILES: CC(=O)OC[C@H](F)C1OC(OP(=O)(O)OP(=O)(S)OC[C@H]2O[C@@H](n3cnc4c(N)ncnc43)[C@H](N(C)C)[C@@H]2O)C(OC(C)=O)C(OC(C)=O)C1OC(C)=O] |
| Example 35B | 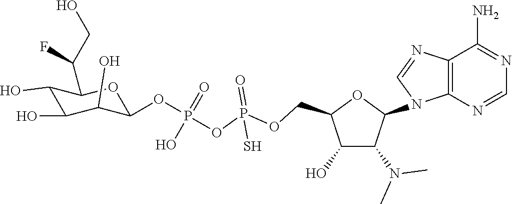 [SMILES: CN(C)[C@@H]1[C@H](O)[C@@H](COP(=O)(S)OP(=O)(O)OC2OC([C@@H](F)CO)C(O)C(O)C2O)O[C@H]1n1cnc2c(N)ncnc21] |
| Example 35C | 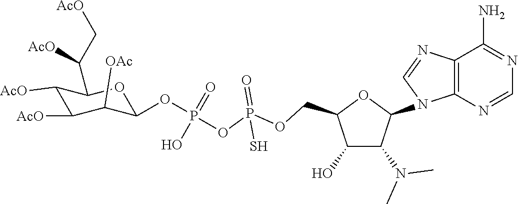 [SMILES: CC(=O)OC[C@H](OC(C)=O)C1OC(OP(=O)(O)OP(=O)(S)OC[C@H]2O[C@@H](n3cnc4c(N)ncnc43)[C@H](N(C)C)[C@@H]2O)C(OC(C)=O)C(OC(C)=O)C1OC(C)=O] |
| Example 35D | 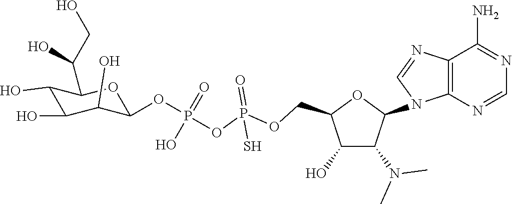 [SMILES: CN(C)[C@@H]1[C@H](O)[C@@H](COP(=O)(S)OP(=O)(O)OC2OC([C@@H](O)CO)C(O)C(O)C2O)O[C@H]1n1cnc2c(N)ncnc21] |
| Example 35E | 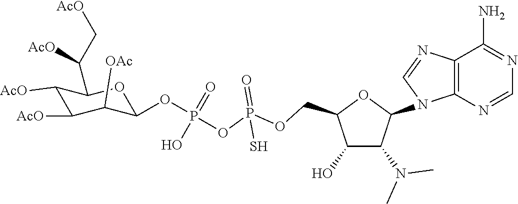 [SMILES: CC(=O)OC[C@H](OC(C)=O)C1OC(OP(=O)(O)OP(=O)(S)OC[C@H]2O[C@@H](n3cnc4c(N)ncnc43)[C@H](N(C)C)[C@@H]2O)C(OC(C)=O)C(OC(C)=O)C1OC(C)=O] |
| Example 35F | 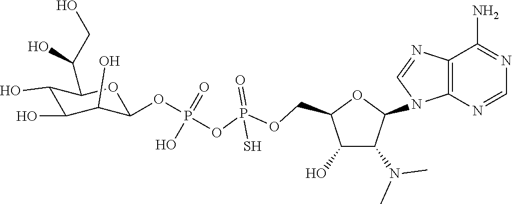 [SMILES: CN(C)[C@@H]1[C@H](O)[C@@H](COP(=O)(S)OP(=O)(O)OC2OC([C@@H](O)CO)C(O)C(O)C2O)O[C@H]1n1cnc2c(N)ncnc21] |
| Example 35G | 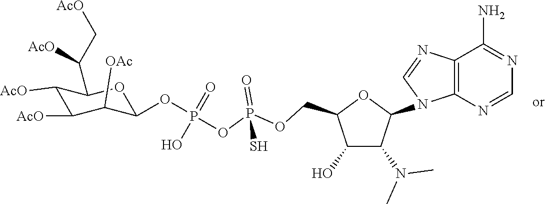 [SMILES: CC(=O)OC[C@H](OC(C)=O)C1OC(OP(=O)(O)O[P@@](=O)(S)OC[C@H]2O[C@@H](n3cnc4c(N)ncnc43)[C@H](N(C)C)[C@@H]2O)C(OC(C)=O)C(OC(C)=O)C1OC(C)=O] |
| | 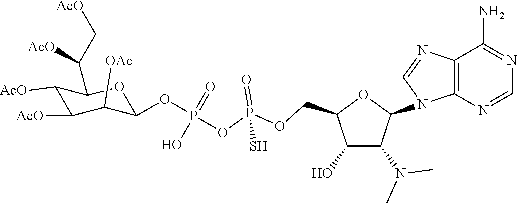 [SMILES: CC(=O)OC[C@H](OC(C)=O)C1OC(OP(=O)(O)O[P@](=O)(S)OC[C@H]2O[C@@H](n3cnc4c(N)ncnc43)[C@H](N(C)C)[C@@H]2O)C(OC(C)=O)C(OC(C)=O)C1OC(C)=O] |
| Example 35H | 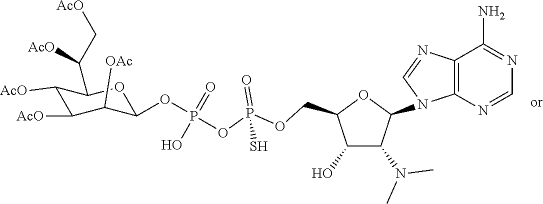 [SMILES: CC(=O)OC[C@H](OC(C)=O)C1OC(OP(=O)(O)O[P@](=O)(S)OC[C@H]2O[C@@H](n3cnc4c(N)ncnc43)[C@H](N(C)C)[C@@H]2O)C(OC(C)=O)C(OC(C)=O)C1OC(C)=O] |
| | 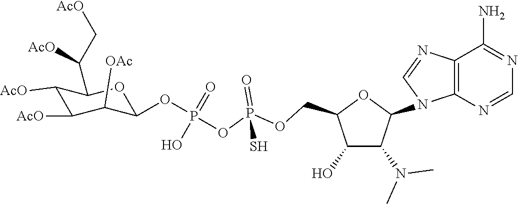 [SMILES: CC(=O)OC[C@H](OC(C)=O)C1OC(OP(=O)(O)O[P@@](=O)(S)OC[C@H]2O[C@@H](n3cnc4c(N)ncnc43)[C@H](N(C)C)[C@@H]2O)C(OC(C)=O)C(OC(C)=O)C1OC(C)=O] |
| Example 35I | 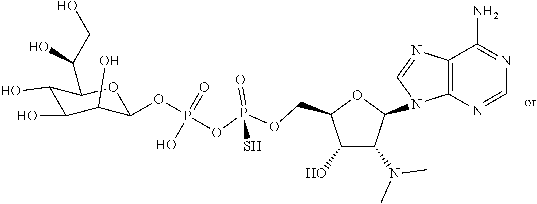 [SMILES: CN(C)[C@@H]1[C@H](O)[C@@H](CO[P@](=O)(S)OP(=O)(O)OC2OC([C@@H](O)CO)C(O)C(O)C2O)O[C@H]1n1cnc2c(N)ncnc21] |
| | 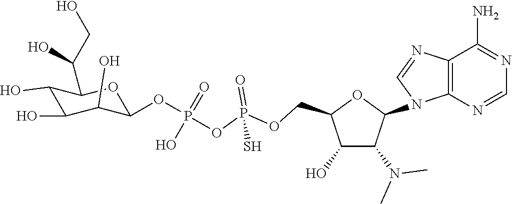 [SMILES: CN(C)[C@@H]1[C@H](O)[C@@H](CO[P@@](=O)(S)OP(=O)(O)OC2OC([C@@H](O)CO)C(O)C(O)C2O)O[C@H]1n1cnc2c(N)ncnc21] |
| Example 35J | 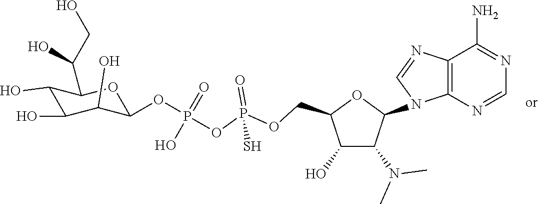 [SMILES: CN(C)[C@@H]1[C@H](O)[C@@H](CO[P@@](=O)(S)OP(=O)(O)OC2OC([C@@H](O)CO)C(O)C(O)C2O)O[C@H]1n1cnc2c(N)ncnc21] |
| | 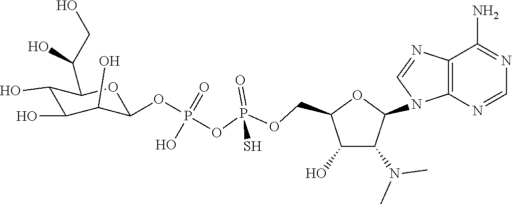 [SMILES: CN(C)[C@@H]1[C@H](O)[C@@H](CO[P@](=O)(S)OP(=O)(O)OC2OC([C@@H](O)CO)C(O)C(O)C2O)O[C@H]1n1cnc2c(N)ncnc21] |
| Example 35K | 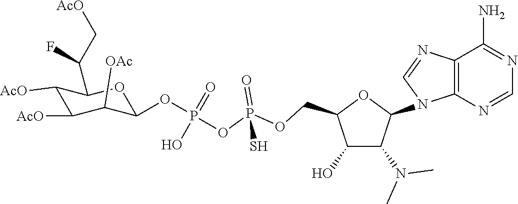 [SMILES: CC(=O)OC[C@H](F)C1OC(OP(=O)(O)O[P@@](=O)(S)OC[C@H]2O[C@@H](n3cnc4c(N)ncnc43)[C@H](N(C)C)[C@@H]2O)C(OC(C)=O)C(OC(C)=O)C1OC(C)=O] |
| Example 35L | 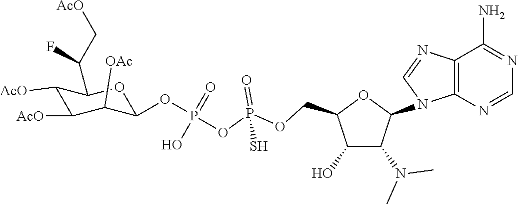 [SMILES: CC(=O)OC[C@H](F)C1OC(OP(=O)(O)O[P@](=O)(S)OC[C@H]2O[C@@H](n3cnc4c(N)ncnc43)[C@H](N(C)C)[C@@H]2O)C(OC(C)=O)C(OC(C)=O)C1OC(C)=O] |
| Example 36A | 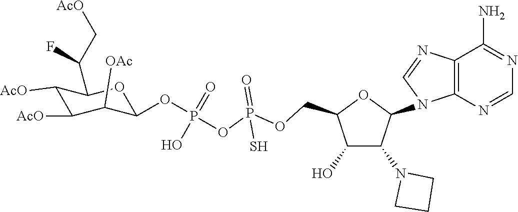 [SMILES: CC(=O)OC[C@H](F)C1OC(OP(=O)(O)OP(=O)(S)OC[C@H]2O[C@@H](n3cnc4c(N)ncnc43)[C@H](N3CCC3)[C@@H]2O)C(OC(C)=O)C(OC(C)=O)C1OC(C)=O] |
| Example 36B | 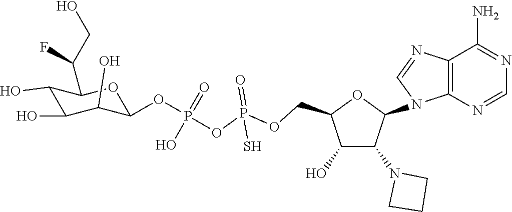 [SMILES: Nc1ncnc2c1ncn2[C@@H]1O[C@H](COP(=O)(S)OP(=O)(O)OC2OC([C@@H](F)CO)C(O)C(O)C2O)[C@@H](O)[C@H]1N1CCC1] |
| Example 36C | 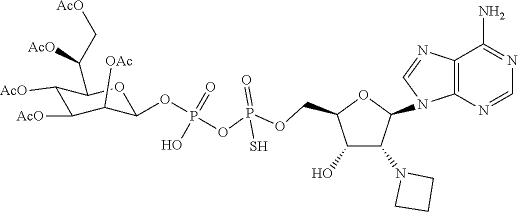 [SMILES: CC(=O)OC[C@H](OC(C)=O)C1OC(OP(=O)(O)OP(=O)(S)OC[C@H]2O[C@@H](n3cnc4c(N)ncnc43)[C@H](N3CCC3)[C@@H]2O)C(OC(C)=O)C(OC(C)=O)C1OC(C)=O] |
| Example 36D | 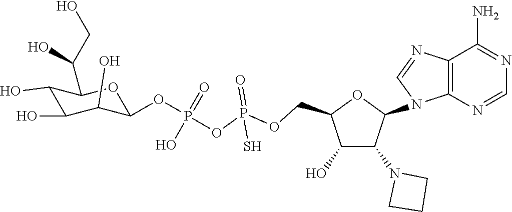 [SMILES: Nc1ncnc2c1ncn2[C@@H]1O[C@H](COP(=O)(S)OP(=O)(O)OC2OC([C@@H](O)CO)C(O)C(O)C2O)[C@@H](O)[C@H]1N1CCC1] |
| Example 37A | 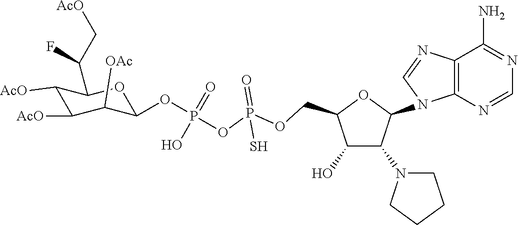 [SMILES: CC(=O)OC[C@H](F)C1OC(OP(=O)(O)OP(=O)(S)OC[C@H]2O[C@@H](n3cnc4c(N)ncnc43)[C@H](N3CCCC3)[C@@H]2O)C(OC(C)=O)C(OC(C)=O)C1OC(C)=O] |
| Example 37B | 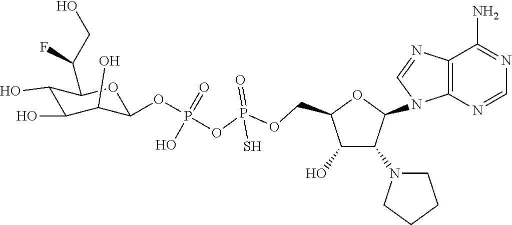 [SMILES: Nc1ncnc2c1ncn2[C@@H]1O[C@H](COP(=O)(S)OP(=O)(O)OC2OC([C@@H](F)CO)C(O)C(O)C2O)[C@@H](O)[C@H]1N1CCCC1] |
| Example 37C | 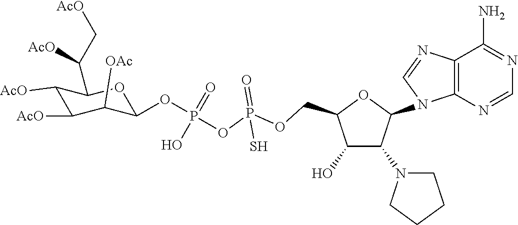 [SMILES: CC(=O)OC[C@H](OC(C)=O)C1OC(OP(=O)(O)OP(=O)(S)OC[C@H]2O[C@@H](n3cnc4c(N)ncnc43)[C@H](N3CCCC3)[C@@H]2O)C(OC(C)=O)C(OC(C)=O)C1OC(C)=O] |
| Example 37D | 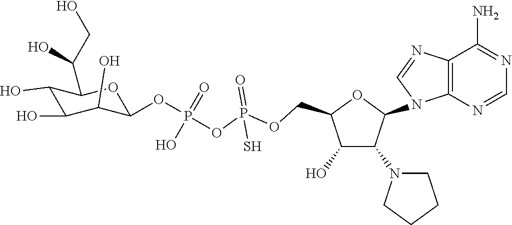 [SMILES: Nc1ncnc2c1ncn2[C@@H]1O[C@H](COP(=O)(S)OP(=O)(O)OC2OC([C@@H](O)CO)C(O)C(O)C2O)[C@@H](O)[C@H]1N1CCCC1] |
| Example 38A | 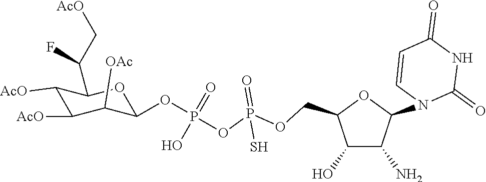 [SMILES: CC(=O)OC[C@H](F)C1OC(OP(=O)(O)OP(=O)(S)OC[C@H]2O[C@@H](n3ccc(=O)[nH]c3=O)[C@H](N)[C@@H]2O)C(OC(C)=O)C(OC(C)=O)C1OC(C)=O] |
| Example 38B | 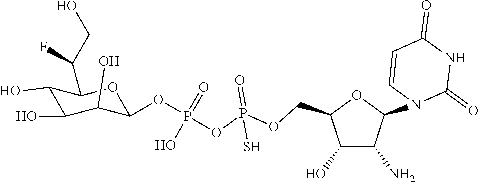 [SMILES: N[C@@H]1[C@H](O)[C@@H](COP(=O)(S)OP(=O)(O)OC2OC([C@@H](F)CO)C(O)C(O)C2O)O[C@H]1n1ccc(=O)[nH]c1=O] |
| Example 38C | 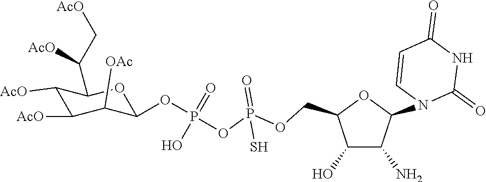 [SMILES: CC(=O)OC[C@H](OC(C)=O)C1OC(OP(=O)(O)OP(=O)(S)OC[C@H]2O[C@@H](n3ccc(=O)[nH]c3=O)[C@H](N)[C@@H]2O)C(OC(C)=O)C(OC(C)=O)C1OC(C)=O] |
| Example 38D | 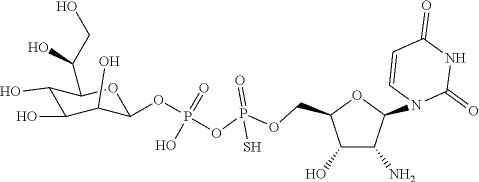 [SMILES: N[C@@H]1[C@H](O)[C@@H](COP(=O)(S)OP(=O)(O)OC2OC([C@@H](O)CO)C(O)C(O)C2O)O[C@H]1n1ccc(=O)[nH]c1=O] |
| Example 39A | 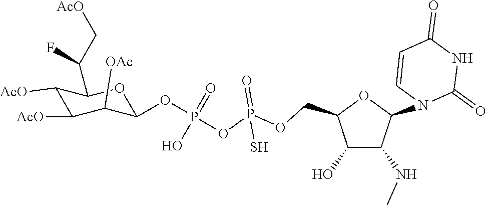 [SMILES: CN[C@@H]1[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1ccc(=O)[nH]c1=O] |
| Example 39B | 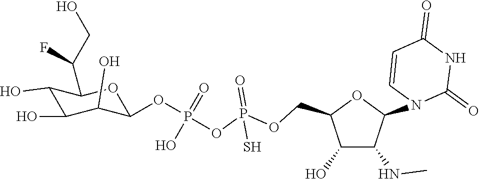 [SMILES: CN[C@@H]1[C@H](O)[C@@H](COP(=O)(S)OP(=O)(O)OC2OC([C@@H](F)CO)C(O)C(O)C2O)O[C@H]1n1ccc(=O)[nH]c1=O] |
| Example 39C | 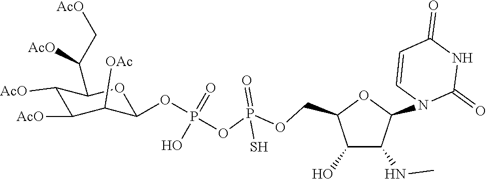 [SMILES: CN[C@@H]1[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1ccc(=O)[nH]c1=O] |
| Example 39D | 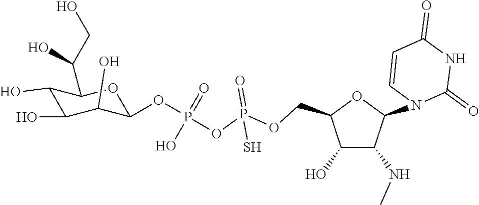 [SMILES: CN[C@@H]1[C@H](O)[C@@H](COP(=O)(S)OP(=O)(O)OC2OC([C@@H](O)CO)C(O)C(O)C2O)O[C@H]1n1ccc(=O)[nH]c1=O] |
| Example 39E | 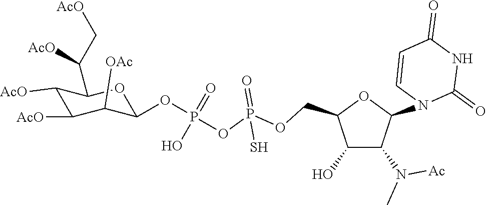 [SMILES: CC(=O)OC[C@H](OC(C)=O)C1OC(OP(=O)(O)OP(=O)(S)OC[C@H]2O[C@@H](n3ccc(=O)[nH]c3=O)[C@H](N(C)C(C)=O)[C@@H]2O)C(OC(C)=O)C(OC(C)=O)C1OC(C)=O] |
| Example 40A | 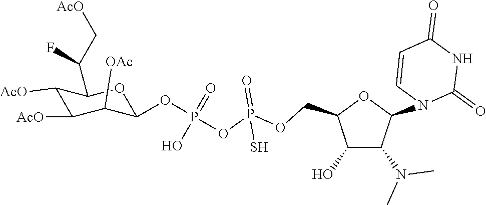 [SMILES: CC(=O)OC[C@H](F)C1OC(OP(=O)(O)OP(=O)(S)OC[C@H]2O[C@@H](n3ccc(=O)[nH]c3=O)[C@H](N(C)C)[C@@H]2O)C(OC(C)=O)C(OC(C)=O)C1OC(C)=O] |
| Example 40B | 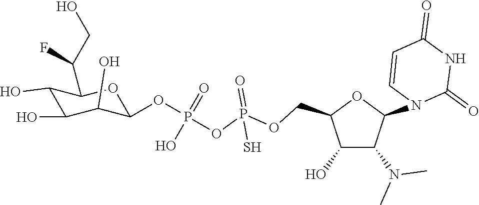 [SMILES: CN(C)[C@@H]1[C@H](O)[C@@H](COP(=O)(S)OP(=O)(O)OC2OC([C@@H](F)CO)C(O)C(O)C2O)O[C@H]1n1ccc(=O)[nH]c1=O] |
| Example 40C | 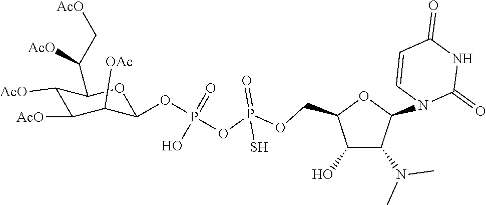 [SMILES: CC(=O)OC[C@H](OC(C)=O)C1OC(OP(=O)(O)OP(=O)(S)OC[C@H]2O[C@@H](n3ccc(=O)[nH]c3=O)[C@H](N(C)C)[C@@H]2O)C(OC(C)=O)C(OC(C)=O)C1OC(C)=O] |
| Example 40D | 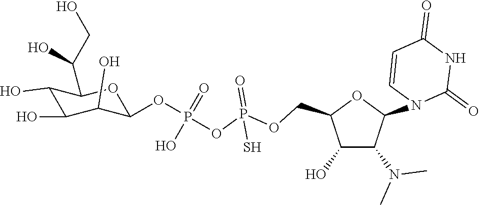 [SMILES: CN(C)[C@@H]1[C@H](O)[C@@H](COP(=O)(S)OP(=O)(O)OC2OC([C@@H](O)CO)C(O)C(O)C2O)O[C@H]1n1ccc(=O)[nH]c1=O] |
| Example 41A | 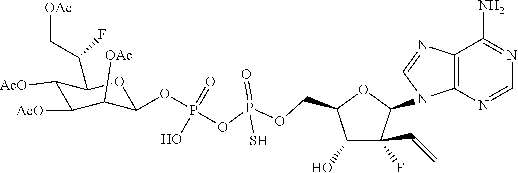 [SMILES: C=C[C@@]1(F)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1cnc2c(N)ncnc21] |
| Example 41B | 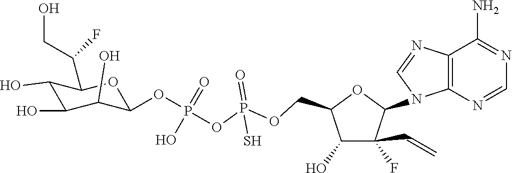 [SMILES: C=C[C@@]1(F)[C@H](O)[C@@H](COP(=O)(S)OP(=O)(O)OC2OC([C@@H](F)CO)C(O)C(O)C2O)O[C@H]1n1cnc2c(N)ncnc21] |
| Example 41C | 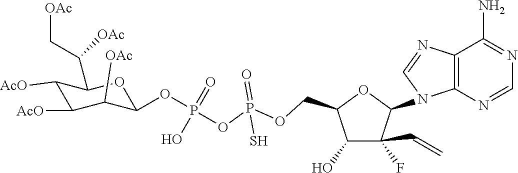 [SMILES: C=C[C@@]1(F)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1cnc2c(N)ncnc21] |
| Example 41D | 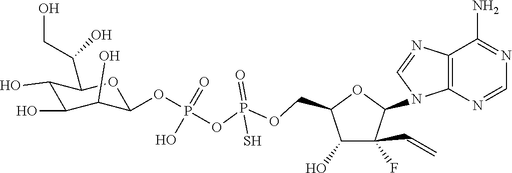 [SMILES: C=C[C@@]1(F)[C@H](O)[C@@H](COP(=O)(S)OP(=O)(O)OC2OC([C@@H](O)CO)C(O)C(O)C2O)O[C@H]1n1cnc2c(N)ncnc21] |
| Example 41G | 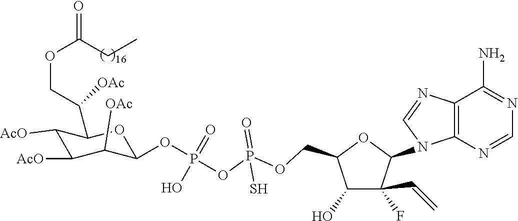 [SMILES: C=C[C@@]1(F)[C@H](O)[C@@H](COP(=O)(S)OP(=O)(O)OC2OC([C@H](COC(=O)CC)OC(C)=O)C(OC(C)=O)C(OC(C)=O)C2OC(C)=O)O[C@H]1n1cnc2c(N)ncnc21] |
| Example 41H | 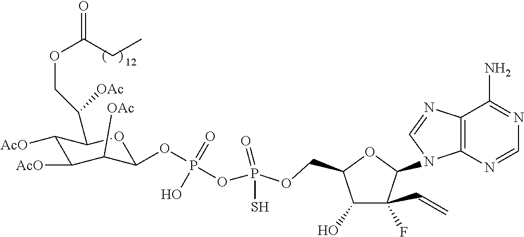 |
| Example 41I | 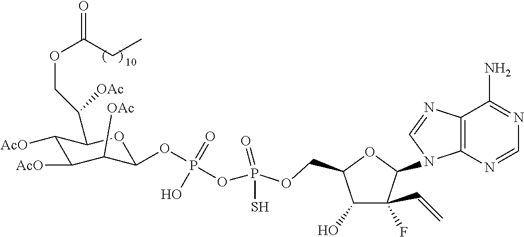 |
| Example 41J | 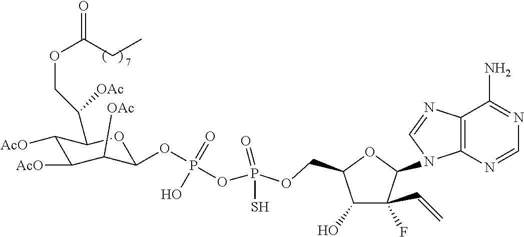 |
| Example 42A | 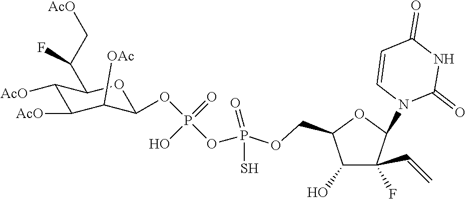 [SMILES: C=C[C@@]1(F)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1ccc(=O)[nH]c1=O] |
| Example 42B | 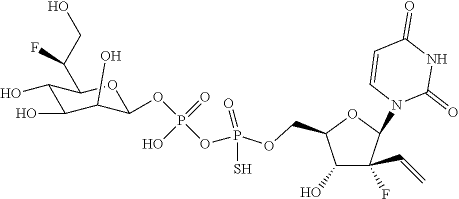 [SMILES: C=C[C@@]1(F)[C@H](O)[C@@H](COP(=O)(S)OP(=O)(O)OC2OC([C@@H](F)CO)C(O)C(O)C2O)O[C@H]1n1ccc(=O)[nH]c1=O] |
| Example 42C | 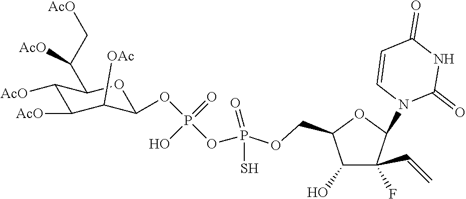 [SMILES: C=C[C@@]1(F)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1ccc(=O)[nH]c1=O] |
| Example 42D | 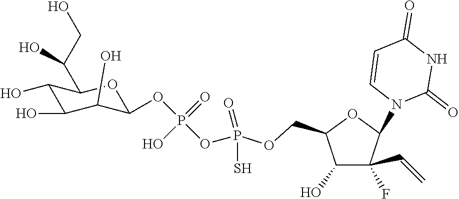 [SMILES: C=C[C@@]1(F)[C@H](O)[C@@H](COP(=O)(S)OP(=O)(O)OC2OC([C@@H](O)CO)C(O)C(O)C2O)O[C@H]1n1ccc(=O)[nH]c1=O] |
| Example 42G | 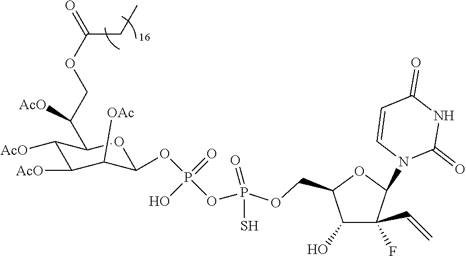 |
| Example 42H | 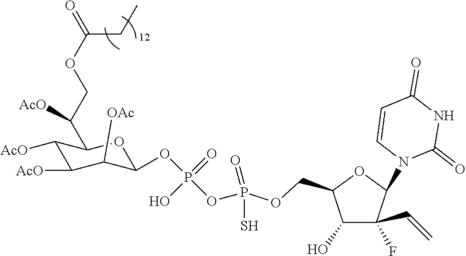 |
| Example 42I | 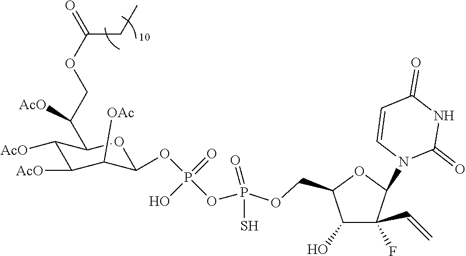 |
| Example 42J | 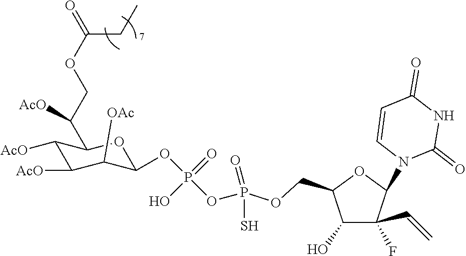 |
| Example 43A | 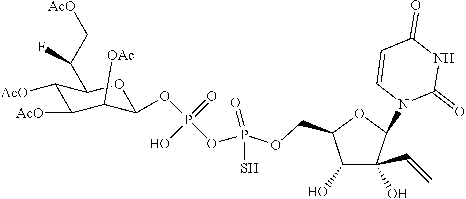 [SMILES: C=C[C@@]1(O)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1ccc(=O)[nH]c1=O] |
| Example 43B | 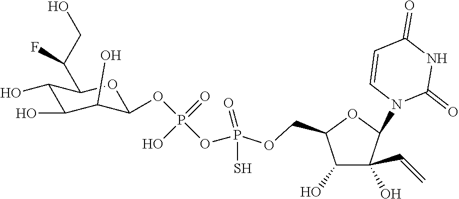 [SMILES: C=C[C@@]1(O)[C@H](O)[C@@H](COP(=O)(S)OP(=O)(O)OC2OC([C@@H](F)CO)C(O)C(O)C2O)O[C@H]1n1ccc(=O)[nH]c1=O] |
| Example 43C | 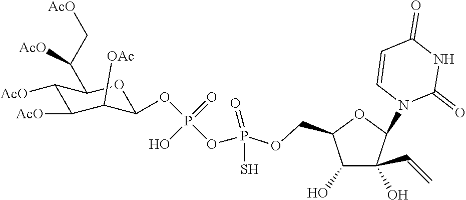 [SMILES: C=C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1ccc(=O)[nH]c1=O] |
| Example 43D | 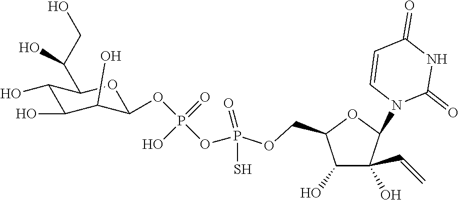 [SMILES: C=C[C@@]1(O)[C@H](O)[C@@H](COP(=O)(S)OP(=O)(O)OC2OC([C@@H](O)CO)C(O)C(O)C2O)O[C@H]1n1ccc(=O)[nH]c1=O] |
| Example 43G | 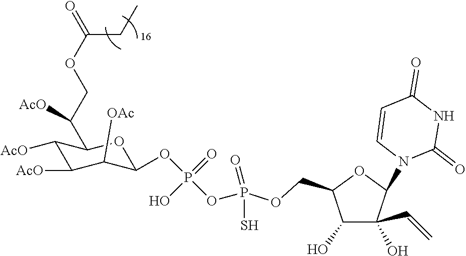 |
| Example 43H | 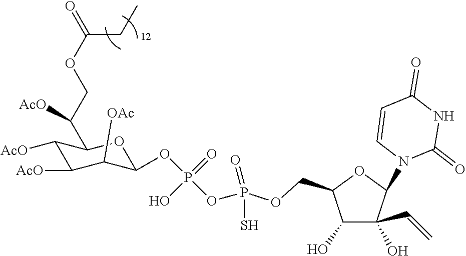 |
| Example 43I | 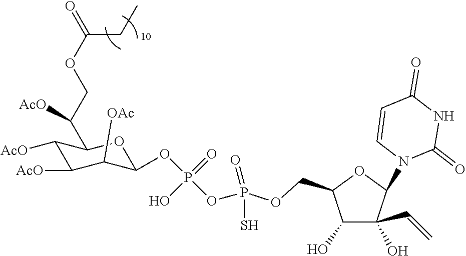 |
| Example 43J | 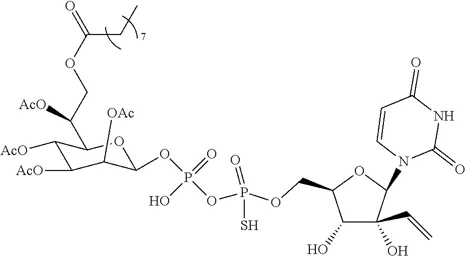 |
| Example 44A | 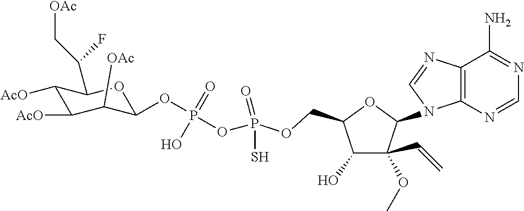 [SMILES: C=C[C@@]1(OC)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1cnc2c(N)ncnc21] |
| Example 44B | 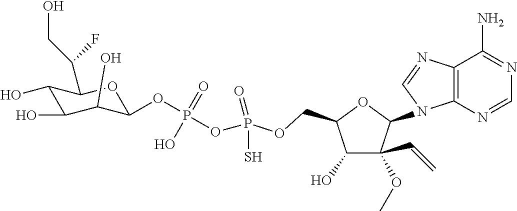 [SMILES: C=C[C@@]1(OC)[C@H](O)[C@@H](COP(=O)(S)OP(=O)(O)OC2OC([C@@H](F)CO)C(O)C(O)C2O)O[C@H]1n1cnc2c(N)ncnc21] |
| Example 44C | 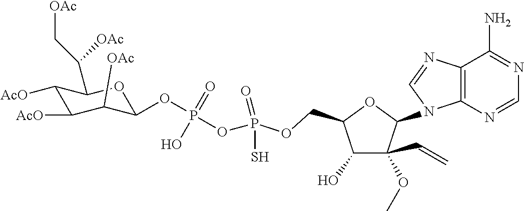 [SMILES: C=C[C@@]1(OC)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1cnc2c(N)ncnc21] |
| Example 44D | 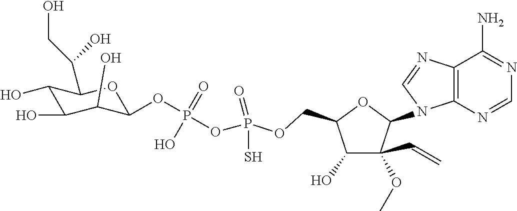 [SMILES: C=C[C@@]1(OC)[C@H](O)[C@@H](COP(=O)(S)OP(=O)(O)OC2OC([C@@H](O)CO)C(O)C(O)C2O)O[C@H]1n1cnc2c(N)ncnc21] |
| Example 44G | 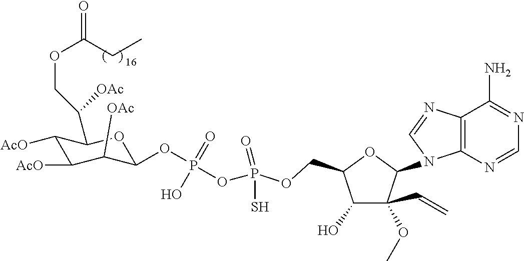 |
| Example 44H | 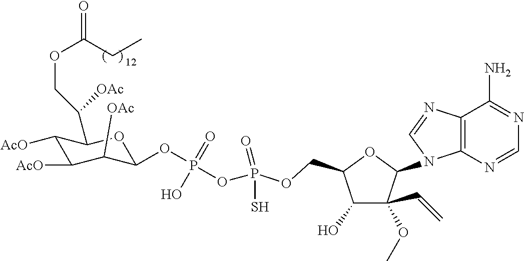 |
| Example 44I | 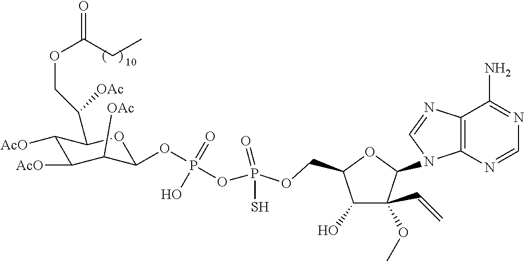 |
| Example 44J | 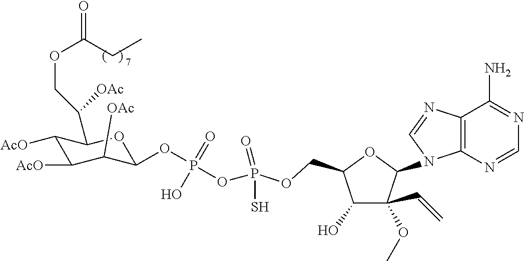 |
| Example 45A | 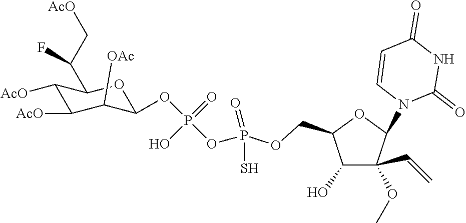 [SMILES: C=C[C@@]1(OC)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1ccc(=O)[nH]c1=O] |
| Example 45B | 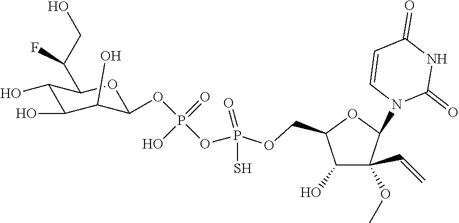 [SMILES: C=C[C@@]1(OC)[C@H](O)[C@@H](COP(=O)(S)OP(=O)(O)OC2OC([C@@H](F)CO)C(O)C(O)C2O)O[C@H]1n1ccc(=O)[nH]c1=O] |
| Example 45C | 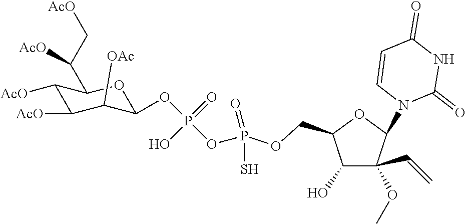 [SMILES: C=C[C@@]1(OC)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1ccc(=O)[nH]c1=O] |
| Example 45D | 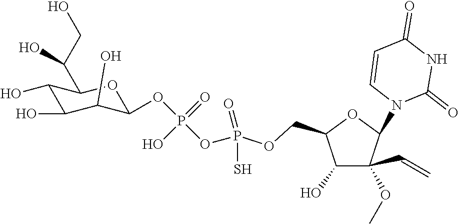 [SMILES: C=C[C@@]1(OC)[C@H](O)[C@@H](COP(=O)(S)OP(=O)(O)OC2OC([C@@H](O)CO)C(O)C(O)C2O)O[C@H]1n1ccc(=O)[nH]c1=O] |
| Example 45E | 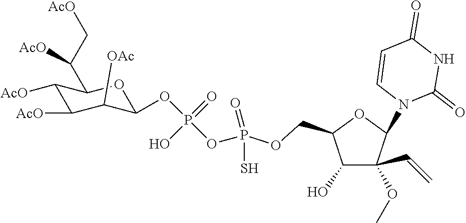 [SMILES: C=C[C@@]1(OC)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1ccc(=O)[nH]c1=O] |
| Example 45G | 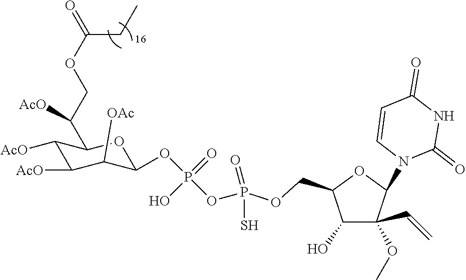 |
| Example 45H | 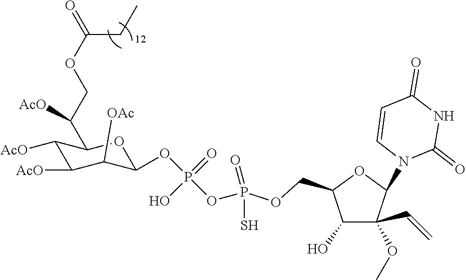 |
| Example 45I | 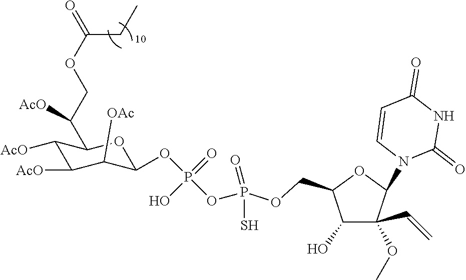 |
| Example 45J | 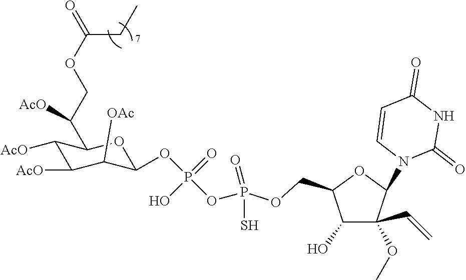 |
| Example 94A | 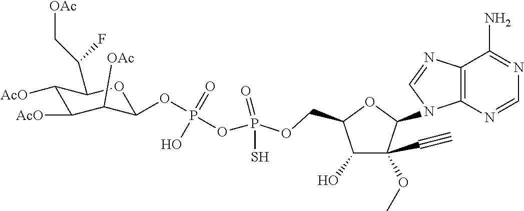 [SMILES: C#C[C@@]1(OC)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1cnc2c(N)ncnc21] |
| Example 94B | 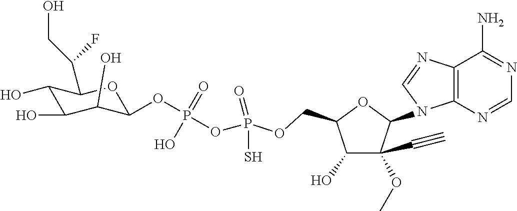 [SMILES: C#C[C@@]1(OC)[C@H](O)[C@@H](COP(=O)(S)OP(=O)(O)OC2OC([C@@H](F)CO)C(O)C(O)C2O)O[C@H]1n1cnc2c(N)ncnc21] |
| Example 94C | 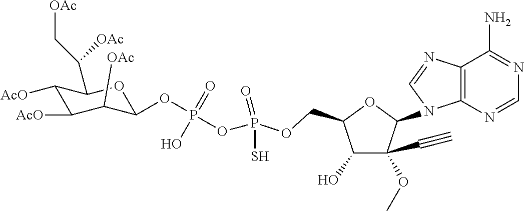 [SMILES: C#C[C@@]1(OC)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1cnc2c(N)ncnc21] |
| Example 94D | 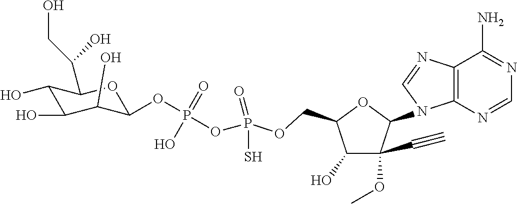 [SMILES: C#C[C@@]1(OC)[C@H](O)[C@@H](COP(=O)(S)OP(=O)(O)OC2OC([C@@H](O)CO)C(O)C(O)C2O)O[C@H]1n1cnc2c(N)ncnc21] |
| Example 95A | 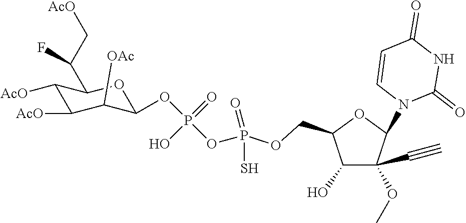 [SMILES: C#C[C@@]1(OC)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1ccc(=O)[nH]c1=O] |
| Example 95B | 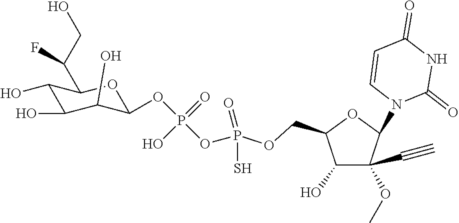 [SMILES: C#C[C@@]1(OC)[C@H](O)[C@@H](COP(=O)(S)OP(=O)(O)OC2OC([C@@H](F)CO)C(O)C(O)C2O)O[C@H]1n1ccc(=O)[nH]c1=O] |
| Example 95C | 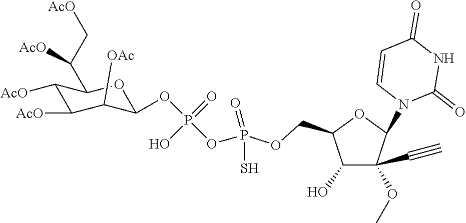 [SMILES: C#C[C@@]1(OC)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1ccc(=O)[nH]c1=O] |
| Example 95D | 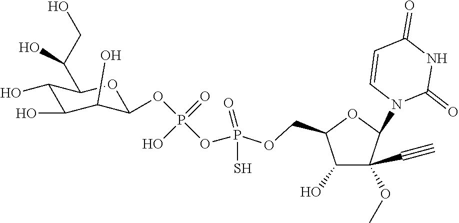 [SMILES: C#C[C@@]1(OC)[C@H](O)[C@@H](COP(=O)(S)OP(=O)(O)OC2OC([C@@H](O)CO)C(O)C(O)C2O)O[C@H]1n1ccc(=O)[nH]c1=O] |
| Example 96A | 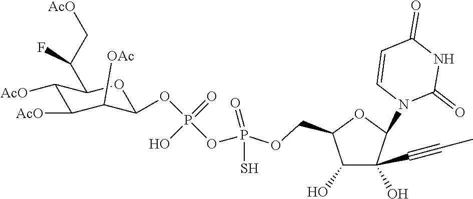 [SMILES: CC#C[C@@]1(O)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1ccc(=O)[nH]c1=O] |
| Example 96B | 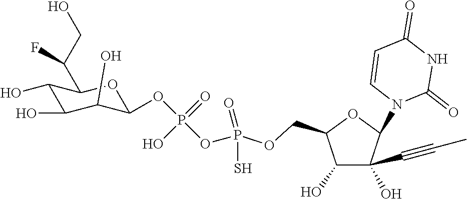 [SMILES: CC#C[C@@]1(O)[C@H](O)[C@@H](COP(=O)(S)OP(=O)(O)OC2OC([C@@H](F)CO)C(O)C(O)C2O)O[C@H]1n1ccc(=O)[nH]c1=O] |
| Example 96C | 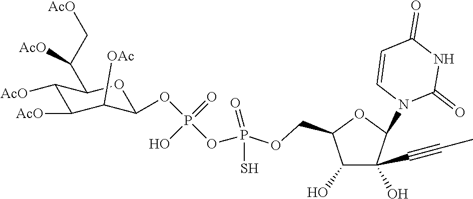 [SMILES: CC#C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1ccc(=O)[nH]c1=O] |
| Example 96D | 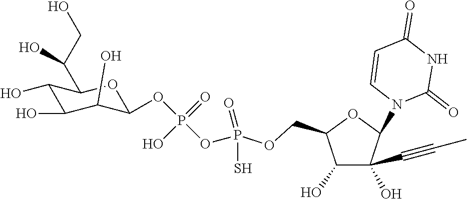 [SMILES: CC#C[C@@]1(O)[C@H](O)[C@@H](COP(=O)(S)OP(=O)(O)OC2OC([C@@H](O)CO)C(O)C(O)C2O)O[C@H]1n1ccc(=O)[nH]c1=O] |
| Example 97A | 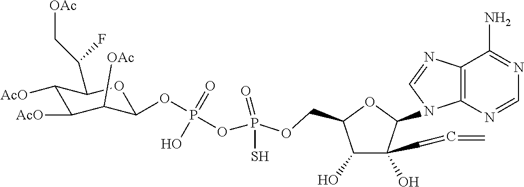 [SMILES: C=C=C[C@@]1(O)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1cnc2c(N)ncnc21] |
| Example 97B | 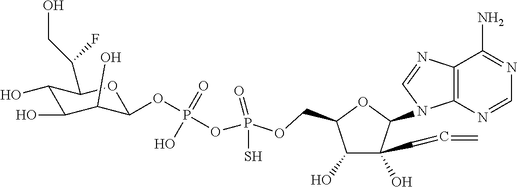 [SMILES: C=C=C[C@@]1(O)[C@H](O)[C@@H](COP(=O)(S)OP(=O)(O)OC2OC([C@@H](F)CO)C(O)C(O)C2O)O[C@H]1n1cnc2c(N)ncnc21] |
| Example 97C | 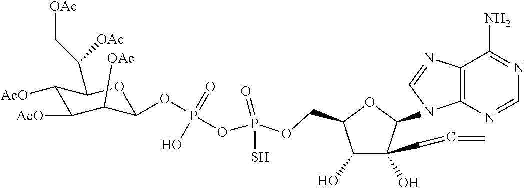 [SMILES: C=C=C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1cnc2c(N)ncnc21] |
| Example 97D | 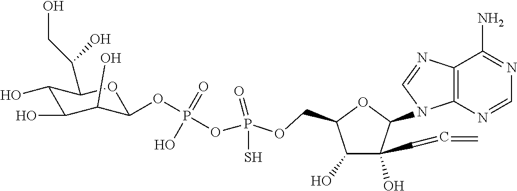 [SMILES: C=C=C[C@@]1(O)[C@H](O)[C@@H](COP(=O)(S)OP(=O)(O)OC2OC([C@@H](O)CO)C(O)C(O)C2O)O[C@H]1n1cnc2c(N)ncnc21] |
| Example 98A | 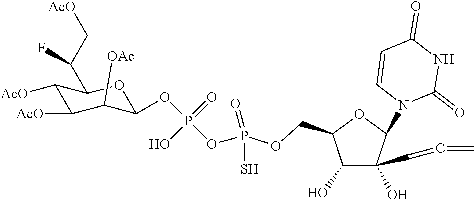 [SMILES: C=C=C[C@@]1(O)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1ccc(=O)[nH]c1=O] |
| Example 98B | 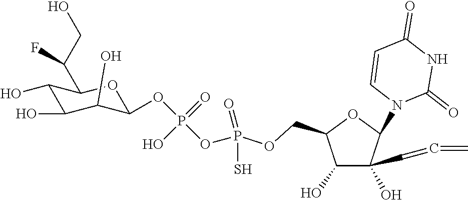 [SMILES: C=C=C[C@@]1(O)[C@H](O)[C@@H](COP(=O)(S)OP(=O)(O)OC2OC([C@@H](F)CO)C(O)C(O)C2O)O[C@H]1n1ccc(=O)[nH]c1=O] |
| Example 98C | 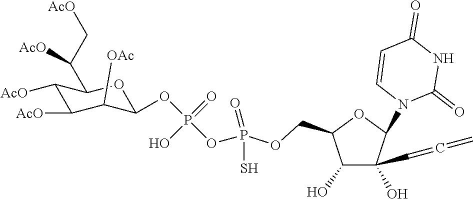 [SMILES: C=C=C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1ccc(=O)[nH]c1=O] |
| Example 98D | 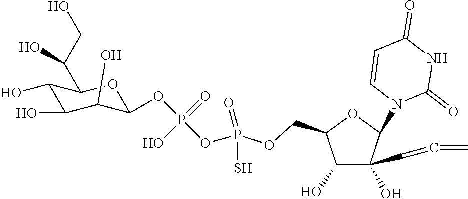 [SMILES: C=C=C[C@@]1(O)[C@H](O)[C@@H](COP(=O)(S)OP(=O)(O)OC2OC([C@@H](O)CO)C(O)C(O)C2O)O[C@H]1n1ccc(=O)[nH]c1=O] |
| Example 99A | 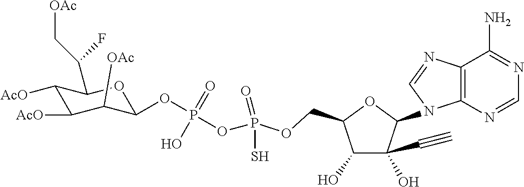 [SMILES: C#C[C@@]1(O)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1cnc2c(N)ncnc21] |
| Example 99B | 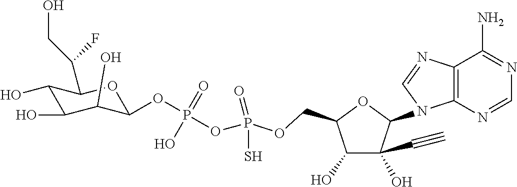 [SMILES: C#C[C@@]1(O)[C@H](O)[C@@H](COP(=O)(S)OP(=O)(O)OC2OC([C@@H](F)CO)C(O)C(O)C2O)O[C@H]1n1cnc2c(N)ncnc21] |
| Example 99C | 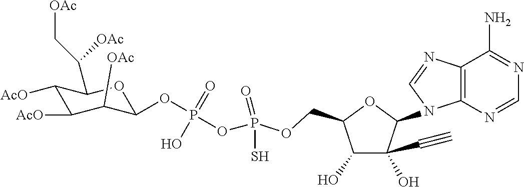 [SMILES: C#C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1cnc2c(N)ncnc21] |
| Example 99D | 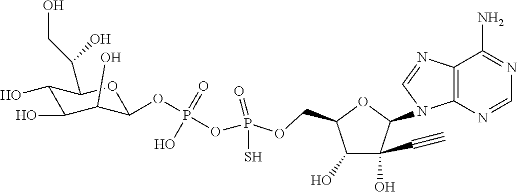 [SMILES: C#C[C@@]1(O)[C@H](O)[C@@H](COP(=O)(S)OP(=O)(O)OC2OC([C@@H](O)CO)C(O)C(O)C2O)O[C@H]1n1cnc2c(N)ncnc21] |
| Example 100A | 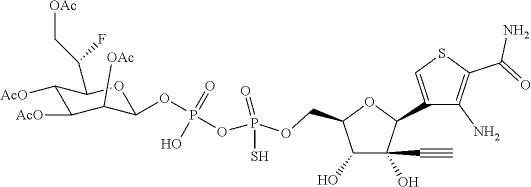 [SMILES: C#C[C@@]1(O)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1csc(C(N)=O)c1N] |
| Example 100B | 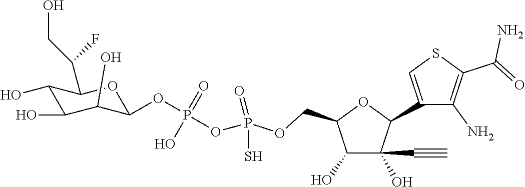 [SMILES: C#C[C@@]1(O)[C@H](O)[C@@H](COP(=O)(S)OP(=O)(O)OC2OC([C@@H](F)CO)C(O)C(O)C2O)O[C@H]1c1csc(C(N)=O)c1N] |
| Example 100C | 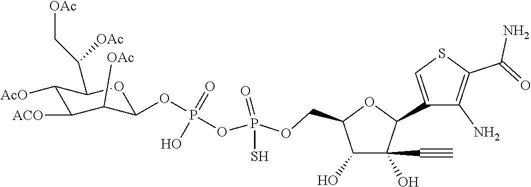 |
| Example 100D | 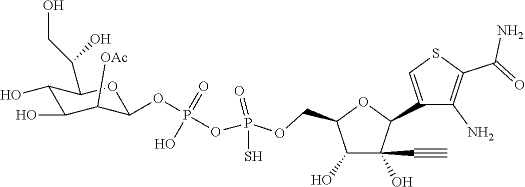 [SMILES: C#C[C@@]1(O)[C@H](O)[C@@H](COP(=O)(S)OP(=O)(O)OC2OC([C@@H](O)CO)C(O)C(O)C2OC(C)=O)O[C@H]1c1csc(C(N)=O)c1N] |
| Example 101A | 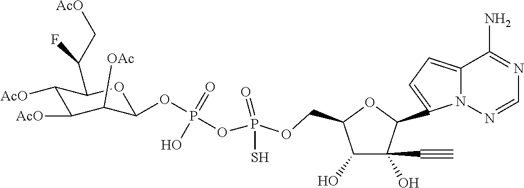 [SMILES: C#C[C@@]1(O)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1ccc2c(N)ncnn12] |
| Example 101B | 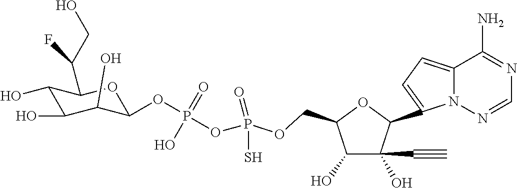 [SMILES: C#C[C@@]1(O)[C@H](O)[C@@H](COP(=O)(S)OP(=O)(O)OC2OC([C@@H](F)CO)C(O)C(O)C2O)O[C@H]1c1ccc2c(N)ncnn12] |
| Example 101C | 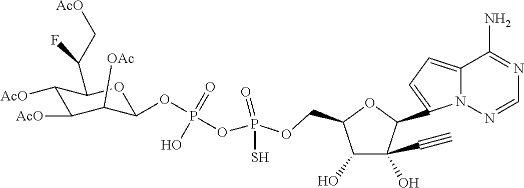 [SMILES: C#C[C@@]1(O)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1ccc2c(N)ncnn12] |
| Example 101D | 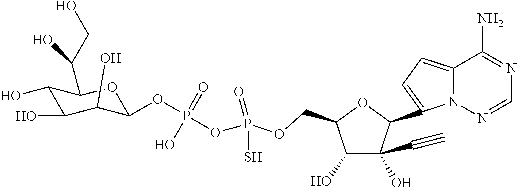 [SMILES: C#C[C@@]1(O)[C@H](O)[C@@H](COP(=O)(S)OP(=O)(O)OC2OC([C@@H](O)CO)C(O)C(O)C2O)O[C@H]1c1ccc2c(N)ncnn12] |
| Example 102A | 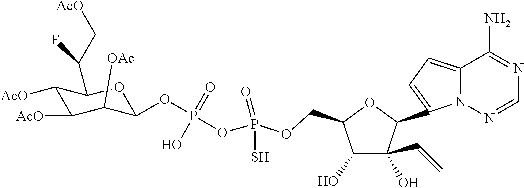 [SMILES: C=C[C@@]1(O)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1ccc2c(N)ncnn12] |
| Example 102B | 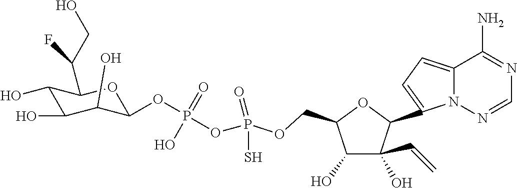 [SMILES: C=C[C@@]1(O)[C@H](O)[C@@H](COP(=O)(S)OP(=O)(O)OC2OC([C@@H](F)CO)C(O)C(O)C2O)O[C@H]1c1ccc2c(N)ncnn12] |
| Example 102C | 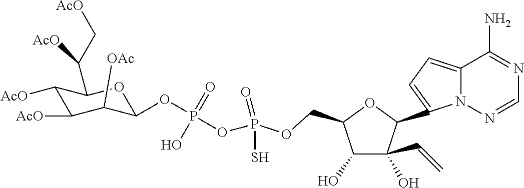 [SMILES: C=C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1ccc2c(N)ncnn12] |
| Example 102D | 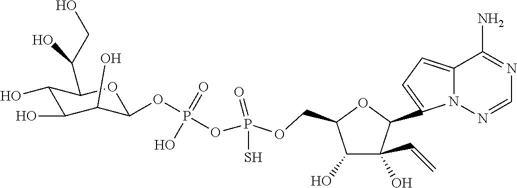 [SMILES: C=C[C@@]1(O)[C@H](O)[C@@H](COP(=O)(S)OP(=O)(O)OC2OC([C@@H](O)CO)C(O)C(O)C2O)O[C@H]1c1ccc2c(N)ncnn12] |
| Example 103A | 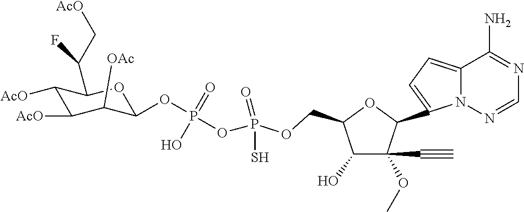 [SMILES: C#C[C@@]1(OC)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1ccc2c(N)ncnn12] |
| Example 103B | 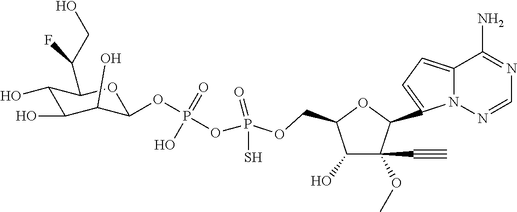 [SMILES: C#C[C@@]1(OC)[C@H](O)[C@@H](COP(=O)(S)OP(=O)(O)OC2OC([C@@H](F)CO)C(O)C(O)C2O)O[C@H]1c1ccc2c(N)ncnn12] |
| Example 103C | 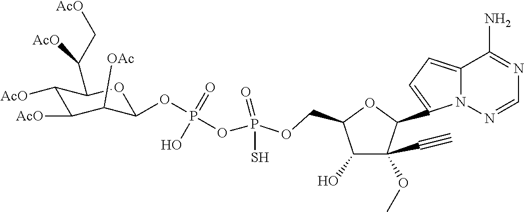 [SMILES: C#C[C@@]1(OC)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1ccc2c(N)ncnn12] |
| Example 103D | 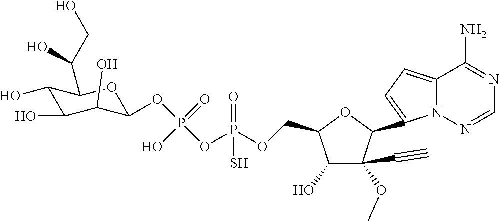 [SMILES: C#C[C@@]1(OC)[C@H](O)[C@@H](COP(=O)(S)OP(=O)(O)OC2OC([C@@H](O)CO)C(O)C(O)C2O)O[C@H]1c1ccc2c(N)ncnn12] |
| Example 108A | 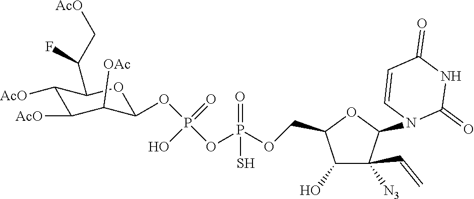 [SMILES: C=C[C@@]1(N=[N+]=[N-])[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1ccc(=O)[nH]c1=O] |
| Example 108B | 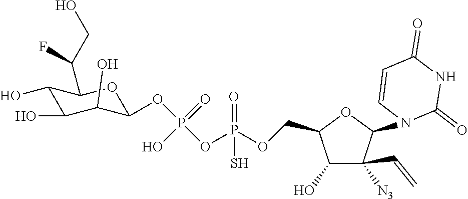 [SMILES: C=C[C@@]1(N=[N+]=[N-])[C@H](O)[C@@H](COP(=O)(S)OP(=O)(O)OC2OC([C@@H](F)CO)C(O)C(O)C2O)O[C@H]1n1ccc(=O)[nH]c1=O] |
| Example 108C | 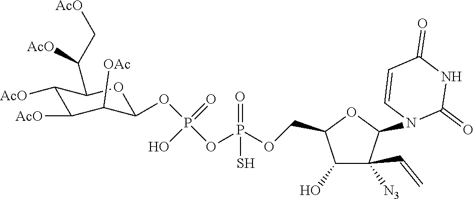 [SMILES: C=C[C@@]1(N=[N+]=[N-])[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1ccc(=O)[nH]c1=O] |
| Example 108D | 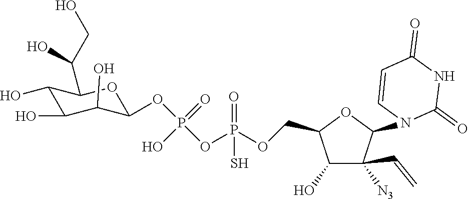 [SMILES: C=C[C@@]1(N=[N+]=[N-])[C@H](O)[C@@H](COP(=O)(S)OP(=O)(O)OC2OC([C@@H](O)CO)C(O)C(O)C2O)O[C@H]1n1ccc(=O)[nH]c1=O] |
| Example 109A | 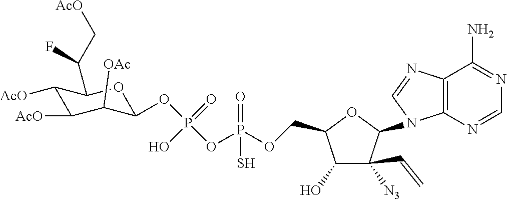 [SMILES: C=C[C@@]1(N=[N+]=[N-])[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1cnc2c(N)ncnc21] |
| Example 109B | 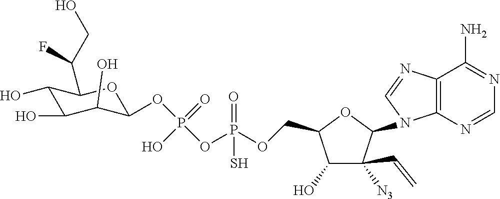 [SMILES: C=C[C@@]1(N=[N+]=[N-])[C@H](O)[C@@H](COP(=O)(S)OP(=O)(O)OC2OC([C@@H](F)CO)C(O)C(O)C2O)O[C@H]1n1cnc2c(N)ncnc21] |
| Example 109C | 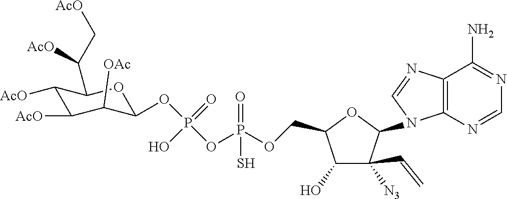 [SMILES: C=C[C@@]1(N=[N+]=[N-])[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1cnc2c(N)ncnc21] |
| Example 109D | 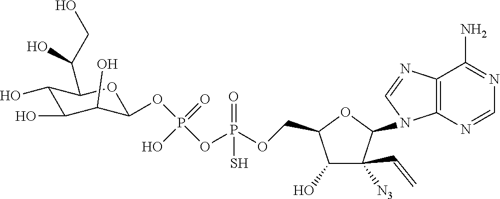 [SMILES: C=C[C@@]1(N=[N+]=[N-])[C@H](O)[C@@H](COP(=O)(S)OP(=O)(O)OC2OC([C@@H](O)CO)C(O)C(O)C2O)O[C@H]1n1cnc2c(N)ncnc21] |
| Example 110A | 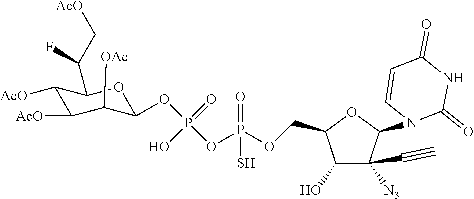 [SMILES: C#C[C@@]1(N=[N+]=[N-])[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1ccc(=O)[nH]c1=O] |
| Example 110B | 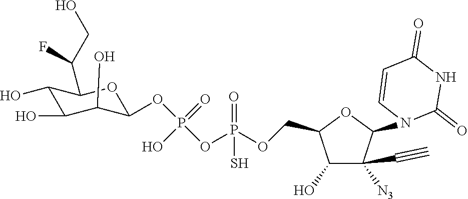 [SMILES: C#C[C@@]1(N=[N+]=[N-])[C@H](O)[C@@H](COP(=O)(S)OP(=O)(O)OC2OC([C@@H](F)CO)C(O)C(O)C2O)O[C@H]1n1ccc(=O)[nH]c1=O] |
| Example 110C | 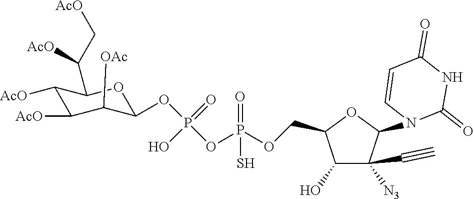 [SMILES: C#C[C@@]1(N=[N+]=[N-])[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1ccc(=O)[nH]c1=O] |
| Example 110D | 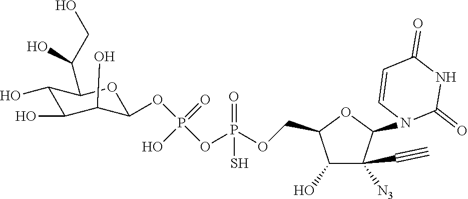 [SMILES: C#C[C@@]1(N=[N+]=[N-])[C@H](O)[C@@H](COP(=O)(S)OP(=O)(O)OC2OC([C@@H](O)CO)C(O)C(O)C2O)O[C@H]1n1ccc(=O)[nH]c1=O] |
| Example 111A | 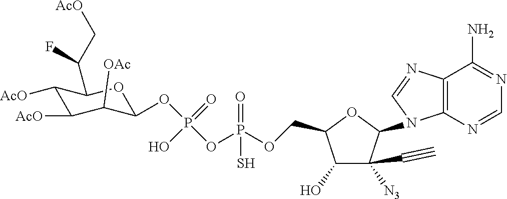 [SMILES: C#C[C@@]1(N=[N+]=[N-])[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1cnc2c(N)ncnc21] |
| Example 111B | 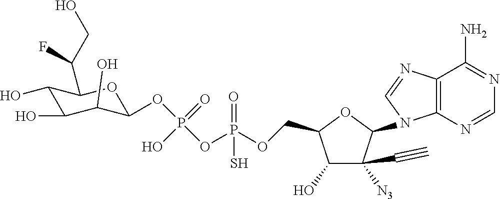 [SMILES: C#C[C@@]1(N=[N+]=[N-])[C@H](O)[C@@H](COP(=O)(S)OP(=O)(O)OC2OC([C@@H](F)CO)C(O)C(O)C2O)O[C@H]1n1cnc2c(N)ncnc21] |
| Example 111C | 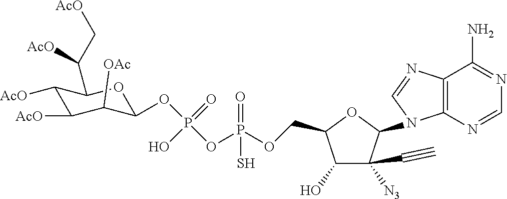 [SMILES: C#C[C@@]1(N=[N+]=[N-])[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1cnc2c(N)ncnc21] |
| Example 111D | 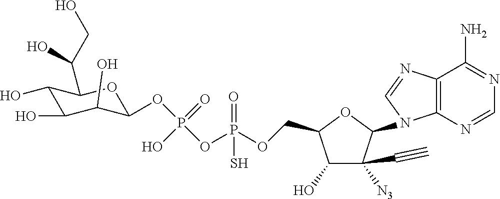 [SMILES: C#C[C@@]1(N=[N+]=[N-])[C@H](O)[C@@H](COP(=O)(S)OP(=O)(O)OC2OC([C@@H](O)CO)C(O)C(O)C2O)O[C@H]1n1cnc2c(N)ncnc21] |
| Example 112A | 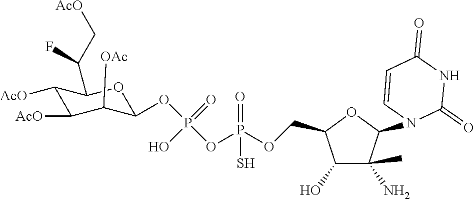 [SMILES: CC(=O)OC[C@H](F)C1OC(OP(=O)(O)OP(=O)(S)OC[C@H]2O[C@@H](n3ccc(=O)[nH]c3=O)[C@](C)(N)[C@@H]2O)C(OC(C)=O)C(OC(C)=O)C1OC(C)=O] |
| Example 112B | 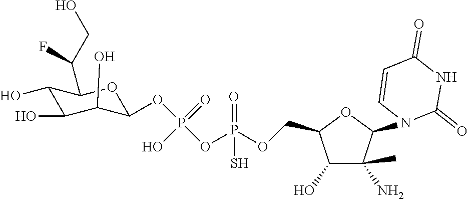 [SMILES: C[C@@]1(N)[C@H](O)[C@@H](COP(=O)(S)OP(=O)(O)OC2OC([C@@H](F)CO)C(O)C(O)C2O)O[C@H]1n1ccc(=O)[nH]c1=O] |
| Example 112C | 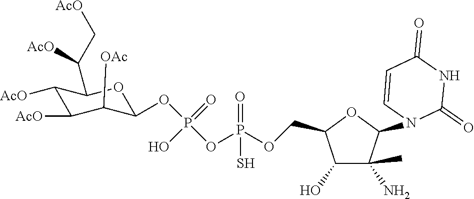 [SMILES: CC(=O)OC[C@H](OC(C)=O)C1OC(OP(=O)(O)OP(=O)(S)OC[C@H]2O[C@@H](n3ccc(=O)[nH]c3=O)[C@](C)(N)[C@@H]2O)C(OC(C)=O)C(OC(C)=O)C1OC(C)=O] |
| Example 112D | 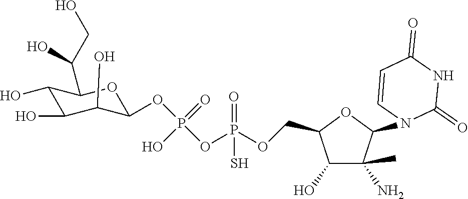 [SMILES: C[C@@]1(N)[C@H](O)[C@@H](COP(=O)(S)OP(=O)(O)OC2OC([C@@H](O)CO)C(O)C(O)C2O)O[C@H]1n1ccc(=O)[nH]c1=O] |
| Example 113A | 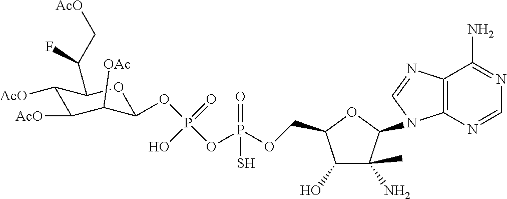 [SMILES: CC(=O)OC[C@H](F)C1OC(OP(=O)(O)OP(=O)(S)OC[C@H]2O[C@@H](n3cnc4c(N)ncnc43)[C@](C)(N)[C@@H]2O)C(OC(C)=O)C(OC(C)=O)C1OC(C)=O] |
| Example 113B | 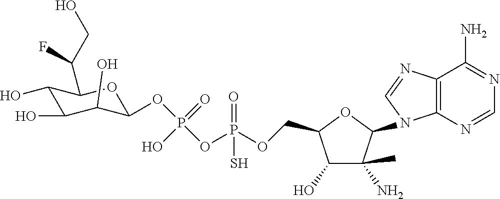 [SMILES: C[C@@]1(N)[C@H](O)[C@@H](COP(=O)(S)OP(=O)(O)OC2OC([C@@H](F)CO)C(O)C(O)C2O)O[C@H]1n1cnc2c(N)ncnc21] |
| Example 113C | 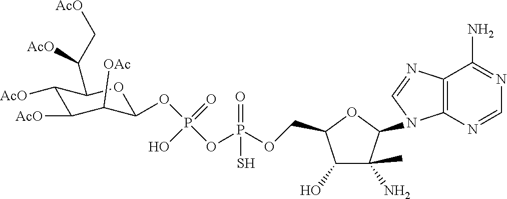 [SMILES: CC(=O)OC[C@H](OC(C)=O)C1OC(OP(=O)(O)OP(=O)(S)OC[C@H]2O[C@@H](n3cnc4c(N)ncnc43)[C@](C)(N)[C@@H]2O)C(OC(C)=O)C(OC(C)=O)C1OC(C)=O] |
| Example 113D | 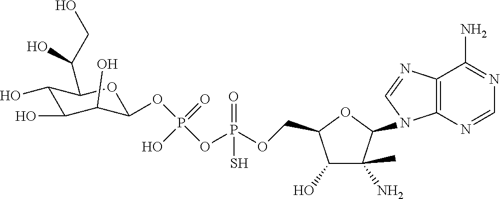 [SMILES: C[C@@]1(N)[C@H](O)[C@@H](COP(=O)(S)OP(=O)(O)OC2OC([C@@H](O)CO)C(O)C(O)C2O)O[C@H]1n1cnc2c(N)ncnc21] |
| Example 114A | 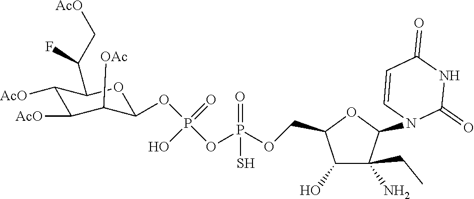 [SMILES: CC[C@@]1(N)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1ccc(=O)[nH]c1=O] |
| Example 114B | 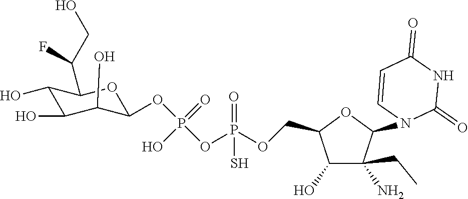 [SMILES: CC[C@@]1(N)[C@H](O)[C@@H](COP(=O)(S)OP(=O)(O)OC2OC([C@@H](F)CO)C(O)C(O)C2O)O[C@H]1n1ccc(=O)[nH]c1=O] |
| Example 114C | 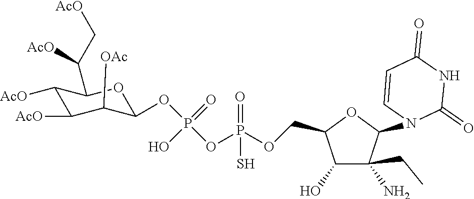 [SMILES: CC[C@@]1(N)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1ccc(=O)[nH]c1=O] |
| Example 114D | 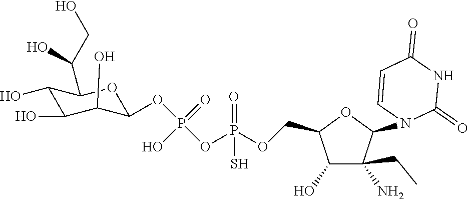 [SMILES: CC[C@@]1(N)[C@H](O)[C@@H](COP(=O)(S)OP(=O)(O)OC2OC([C@@H](O)CO)C(O)C(O)C2O)O[C@H]1n1ccc(=O)[nH]c1=O] |
| Example 115A | 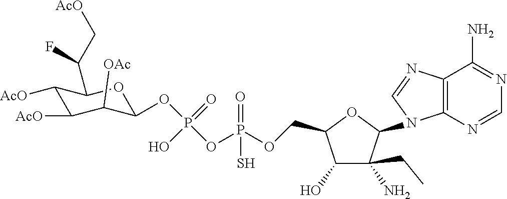 [SMILES: CC[C@@]1(N)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1cnc2c(N)ncnc21] |
| Example 115B | 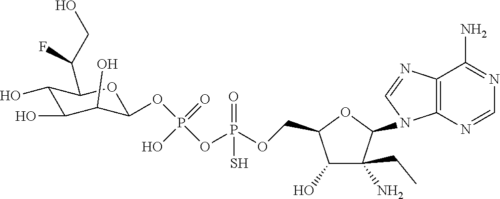 [SMILES: CC[C@@]1(N)[C@H](O)[C@@H](COP(=O)(S)OP(=O)(O)OC2OC([C@@H](F)CO)C(O)C(O)C2O)O[C@H]1n1cnc2c(N)ncnc21] |
| Example 115C | 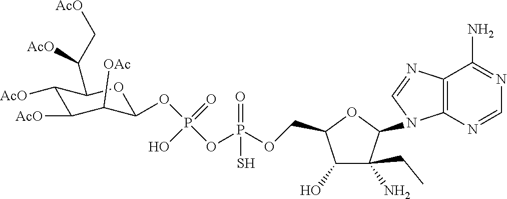 [SMILES: CC[C@@]1(N)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1cnc2c(N)ncnc21] |
| Example 115D | 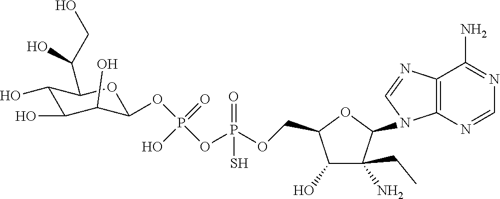 [SMILES: CC[C@@]1(N)[C@H](O)[C@@H](COP(=O)(S)OP(=O)(O)OC2OC([C@@H](O)CO)C(O)C(O)C2O)O[C@H]1n1cnc2c(N)ncnc21] |
| Example 116A | 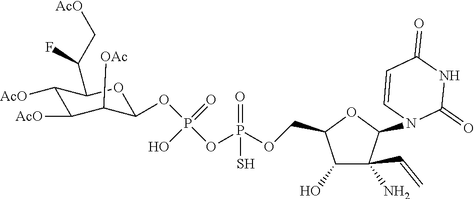 [SMILES: C=C[C@@]1(N)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1ccc(=O)[nH]c1=O] |
| Example 116B | 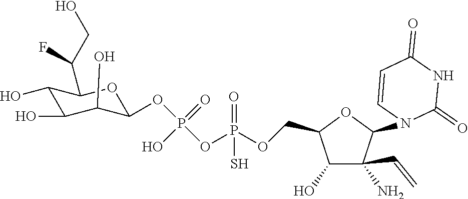 [SMILES: C=C[C@@]1(N)[C@H](O)[C@@H](COP(=O)(S)OP(=O)(O)OC2OC([C@@H](F)CO)C(O)C(O)C2O)O[C@H]1n1ccc(=O)[nH]c1=O] |
| Example 116C | 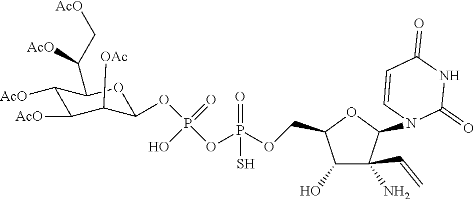 [SMILES: C=C[C@@]1(N)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1ccc(=O)[nH]c1=O] |
| Example 116D | 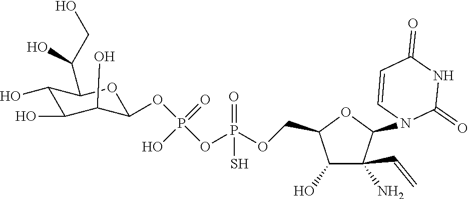 [SMILES: C=C[C@@]1(N)[C@H](O)[C@@H](COP(=O)(S)OP(=O)(O)OC2OC([C@@H](O)CO)C(O)C(O)C2O)O[C@H]1n1ccc(=O)[nH]c1=O] |
| Example 117A | 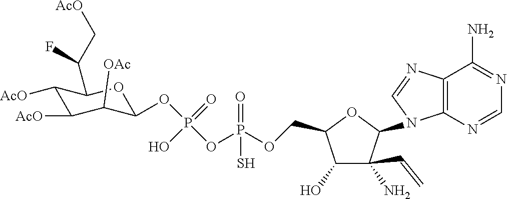 [SMILES: C=C[C@@]1(N)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1cnc2c(N)ncnc21] |
| Example 117B | 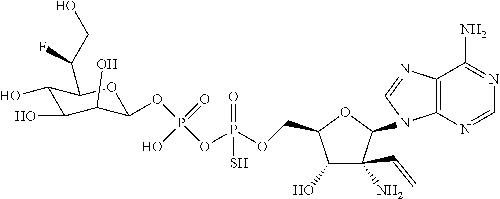 [SMILES: C=C[C@@]1(N)[C@H](O)[C@@H](COP(=O)(S)OP(=O)(O)OC2OC([C@@H](F)CO)C(O)C(O)C2O)O[C@H]1n1cnc2c(N)ncnc21] |
| Example 117C | 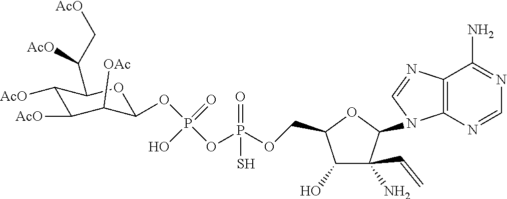 [SMILES: C=C[C@@]1(N)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1cnc2c(N)ncnc21] |
| Example 117D | 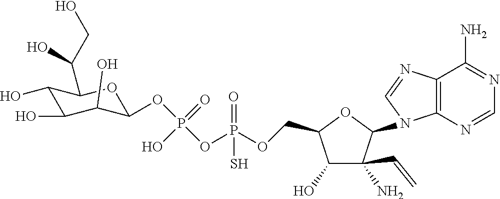 [SMILES: C=C[C@@]1(N)[C@H](O)[C@@H](COP(=O)(S)OP(=O)(O)OC2OC([C@@H](O)CO)C(O)C(O)C2O)O[C@H]1n1cnc2c(N)ncnc21] |
| Example 118A | 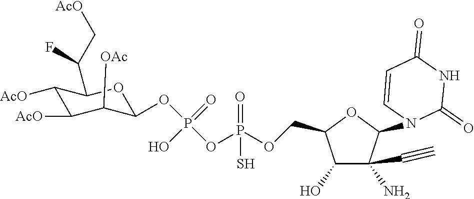 [SMILES: C#C[C@@]1(N)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1ccc(=O)[nH]c1=O] |
| Example 118B | 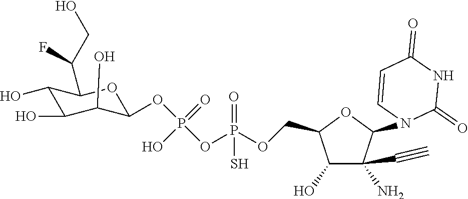 [SMILES: C#C[C@@]1(N)[C@H](O)[C@@H](COP(=O)(S)OP(=O)(O)OC2OC([C@@H](F)CO)C(O)C(O)C2O)O[C@H]1n1ccc(=O)[nH]c1=O] |
| Example 118C | 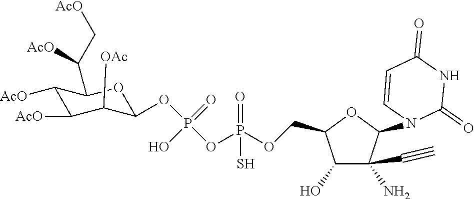 [SMILES: C#C[C@@]1(N)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1ccc(=O)[nH]c1=O] |
| Example 118D | 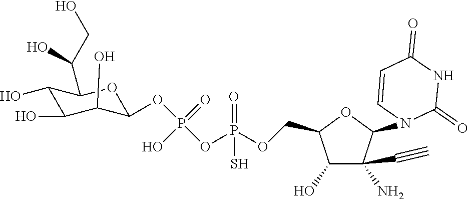 [SMILES: C#C[C@@]1(N)[C@H](O)[C@@H](COP(=O)(S)OP(=O)(O)OC2OC([C@@H](O)CO)C(O)C(O)C2O)O[C@H]1n1ccc(=O)[nH]c1=O] |
| Example 119A | 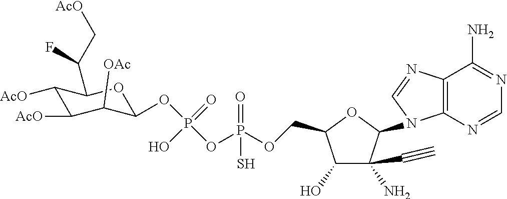 [SMILES: C#C[C@@]1(N)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1cnc2c(N)ncnc21] |
| Example 119B | 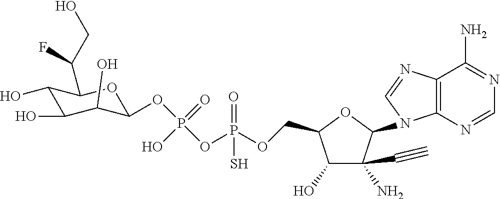 [SMILES: C#C[C@@]1(N)[C@H](O)[C@@H](COP(=O)(S)OP(=O)(O)OC2OC([C@@H](F)CO)C(O)C(O)C2O)O[C@H]1n1cnc2c(N)ncnc21] |
| Example 119C | 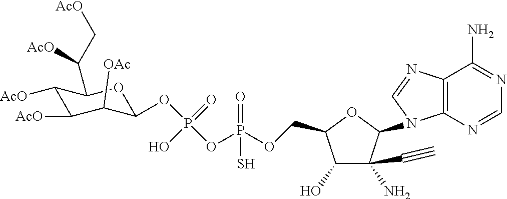 [SMILES: C#C[C@@]1(N)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1cnc2c(N)ncnc21] |
| Example 119D | 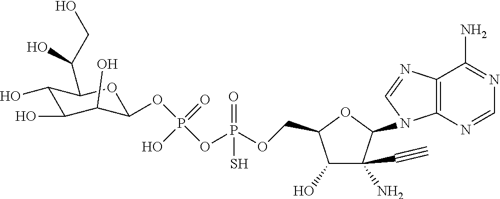 [SMILES: C#C[C@@]1(N)[C@H](O)[C@@H](COP(=O)(S)OP(=O)(O)OC2OC([C@@H](O)CO)C(O)C(O)C2O)O[C@H]1n1cnc2c(N)ncnc21] |
| Example 120A | 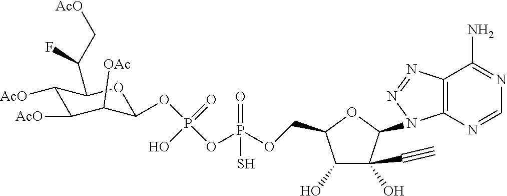 [SMILES: C#C[C@@]1(O)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1nnc2c(N)ncnc21] |
| Example 120B | 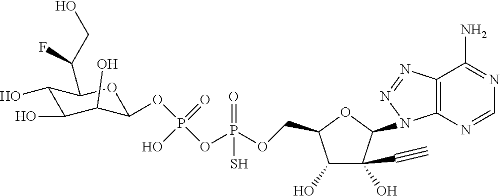 [SMILES: C#C[C@@]1(O)[C@H](O)[C@@H](COP(=O)(S)OP(=O)(O)OC2OC([C@@H](F)CO)C(O)C(O)C2O)O[C@H]1n1nnc2c(N)ncnc21] |
| Example 120C | 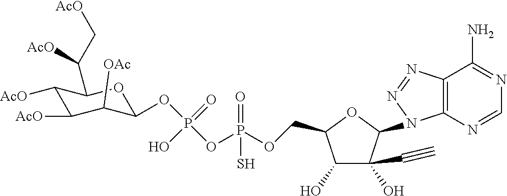 [SMILES: C#C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1nnc2c(N)ncnc21] |
| Example 120D | 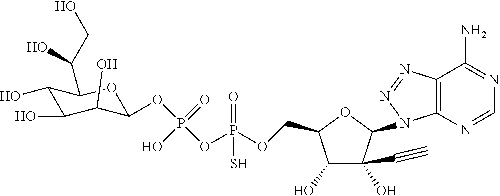 [SMILES: C#C[C@@]1(O)[C@H](O)[C@@H](COP(=O)(S)OP(=O)(O)OC2OC([C@@H](O)CO)C(O)C(O)C2O)O[C@H]1n1nnc2c(N)ncnc21] |
| Example 122A | 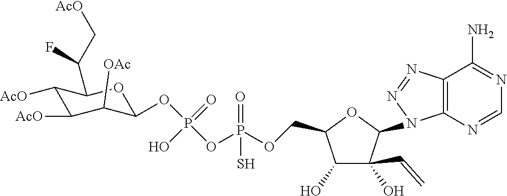 [SMILES: C=C[C@@]1(O)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1nnc2c(N)ncnc21] |
| Example 122B | 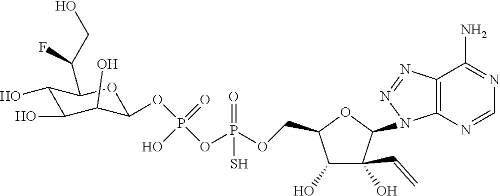 [SMILES: C=C[C@@]1(O)[C@H](O)[C@@H](COP(=O)(S)OP(=O)(O)OC2OC([C@@H](F)CO)C(O)C(O)C2O)O[C@H]1n1nnc2c(N)ncnc21] |
| Example 122C | 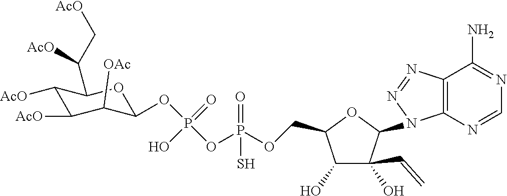 [SMILES: C=C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1nnc2c(N)ncnc21] |
| Example 122D | 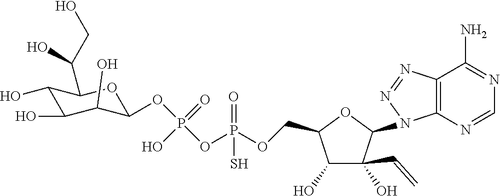 [SMILES: C=C[C@@]1(O)[C@H](O)[C@@H](COP(=O)(S)OP(=O)(O)OC2OC([C@@H](O)CO)C(O)C(O)C2O)O[C@H]1n1nnc2c(N)ncnc21] |
| Example 123A | 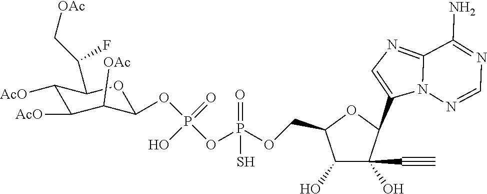 [SMILES: C#C[C@@]1(O)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1cnc2c(N)ncnn12] |
| Example 123B | 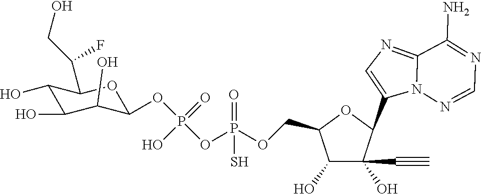 [SMILES: C#C[C@@]1(O)[C@H](O)[C@@H](COP(=O)(S)OP(=O)(O)OC2OC([C@@H](F)CO)C(O)C(O)C2O)O[C@H]1c1cnc2c(N)ncnn12] |
| Example 123C | 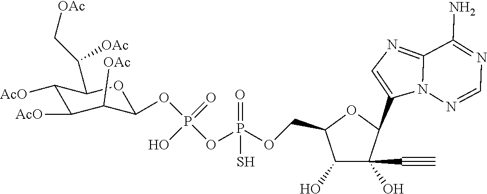 [SMILES: C#C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1cnc2c(N)ncnn12] |
| Example 123D | 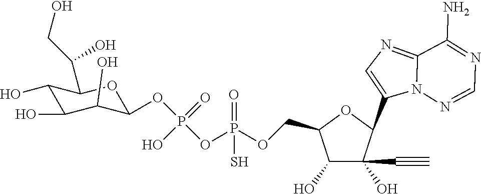 [SMILES: C#C[C@@]1(O)[C@H](O)[C@@H](COP(=O)(S)OP(=O)(O)OC2OC([C@@H](O)CO)C(O)C(O)C2O)O[C@H]1c1cnc2c(N)ncnn12] |
| Example 124A | 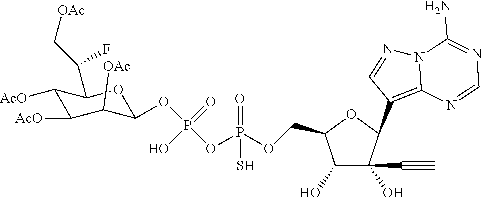 [SMILES: C#C[C@@]1(O)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1cnn2c(N)ncnc12] |
| Example 124B | 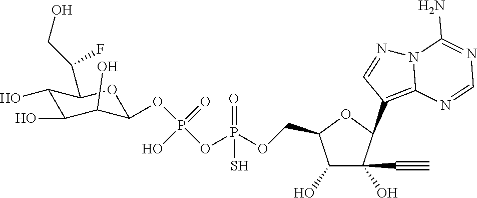 [SMILES: C#C[C@@]1(O)[C@H](O)[C@@H](COP(=O)(S)OP(=O)(O)OC2OC([C@@H](F)CO)C(O)C(O)C2O)O[C@H]1c1cnn2c(N)ncnc12] |
| Example 124C | 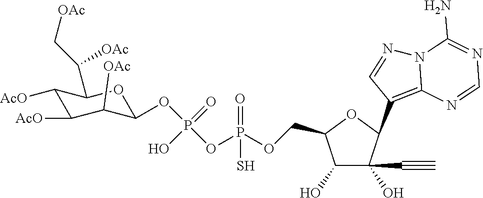 [SMILES: C#C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1cnn2c(N)ncnc12] |
| Example 124D | 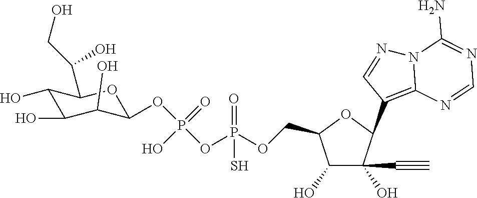 [SMILES: C#C[C@@]1(O)[C@H](O)[C@@H](COP(=O)(S)OP(=O)(O)OC2OC([C@@H](O)CO)C(O)C(O)C2O)O[C@H]1c1cnn2c(N)ncnc12] |
| Example 125A | 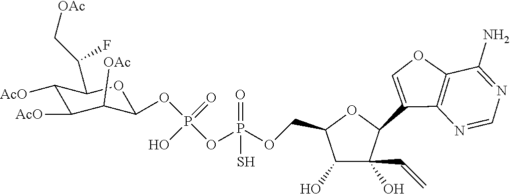 [SMILES: C=C[C@@]1(O)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1coc2c(N)ncnc12] |
| Example 125B | 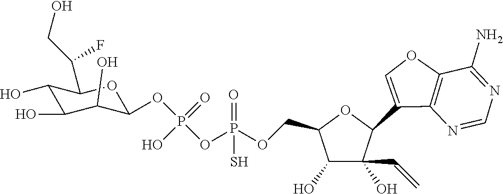 [SMILES: C=C[C@@]1(O)[C@H](O)[C@@H](COP(=O)(S)OP(=O)(O)OC2OC([C@@H](F)CO)C(O)C(O)C2O)O[C@H]1c1coc2c(N)ncnc12] |
| Example 125C | 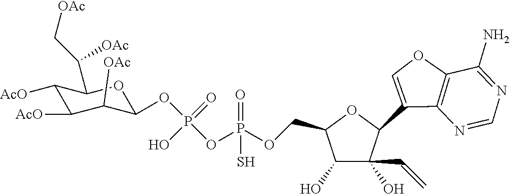 [SMILES: C=C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1coc2c(N)ncnc12] |
| Example 125D | 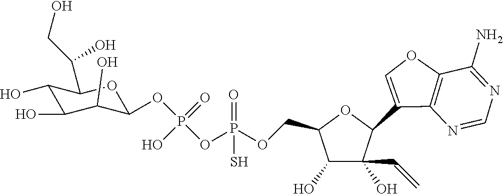 [SMILES: C=C[C@@]1(O)[C@H](O)[C@@H](COP(=O)(S)OP(=O)(O)OC2OC([C@@H](O)CO)C(O)C(O)C2O)O[C@H]1c1coc2c(N)ncnc12] |
| Example 126A | 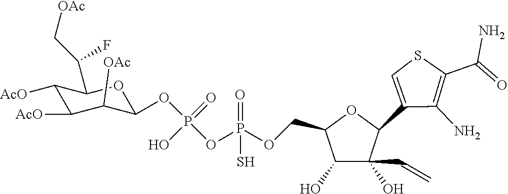 [SMILES: C=C[C@@]1(O)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1csc(C(N)=O)c1N] |
| Example 126B | 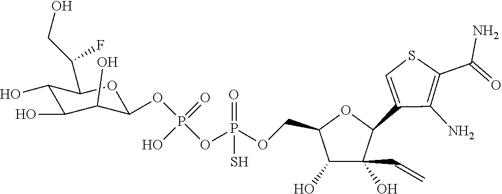 [SMILES: C=C[C@@]1(O)[C@H](O)[C@@H](COP(=O)(S)OP(=O)(O)OC2OC([C@@H](F)CO)C(O)C(O)C2O)O[C@H]1c1csc(C(N)=O)c1N] |
| Example 126C | 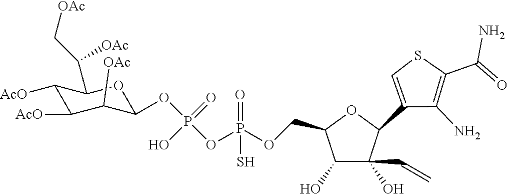 [SMILES: C=C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1csc(C(N)=O)c1N] |
| Example 126D | 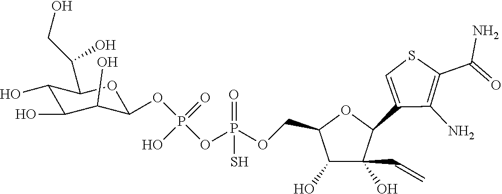 [SMILES: C=C[C@@]1(O)[C@H](O)[C@@H](COP(=O)(S)OP(=O)(O)OC2OC([C@@H](O)CO)C(O)C(O)C2O)O[C@H]1c1csc(C(N)=O)c1N] |
| Example 127A | 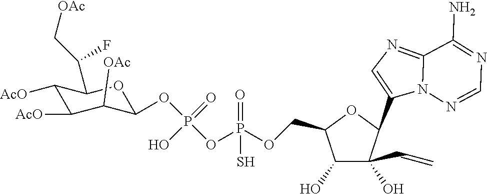 [SMILES: C=C[C@@]1(O)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1cnc2c(N)ncnn12] |
| Example 127B | 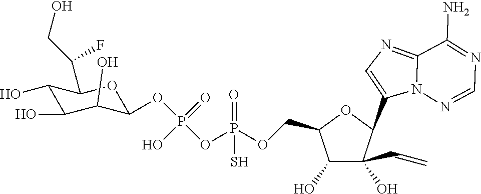 [SMILES: C=C[C@@]1(O)[C@H](O)[C@@H](COP(=O)(S)OP(=O)(O)OC2OC([C@@H](F)CO)C(O)C(O)C2O)O[C@H]1c1cnc2c(N)ncnn12] |
| Example 127C | 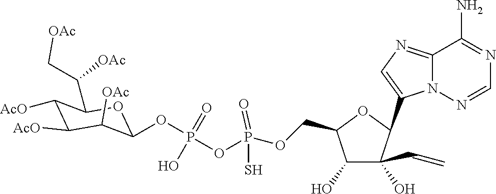 [SMILES: C=C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1cnc2c(N)ncnn12] |
| Example 127D | 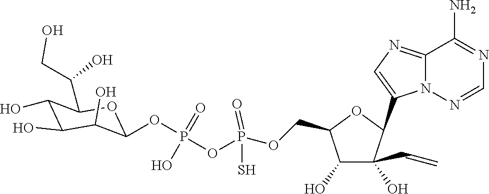 [SMILES: C=C[C@@]1(O)[C@H](O)[C@@H](COP(=O)(S)OP(=O)(O)OC2OC([C@@H](O)CO)C(O)C(O)C2O)O[C@H]1c1cnc2c(N)ncnn12] |
| Example 127E | 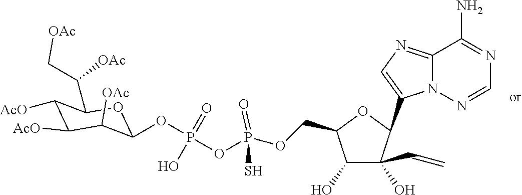 [SMILES: C=C[C@@]1(O)[C@H](O)[C@@H](CO[P@](=O)(S)OP(=O)(O)OC2OC([C@H](COC(C)=O)OC(C)=O)C(OC(C)=O)C(OC(C)=O)C2OC(C)=O)O[C@H]1c1cnc2c(N)ncnn12] |
| | 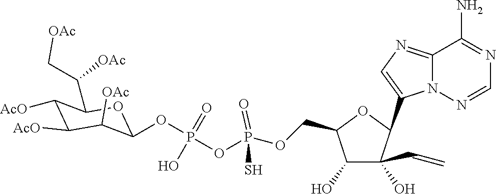 [SMILES: C=C[C@@]1(O)[C@H](O)[C@@H](CO[P@](=O)(S)OP(=O)(O)OC2OC([C@H](COC(C)=O)OC(C)=O)C(OC(C)=O)C(OC(C)=O)C2OC(C)=O)O[C@H]1c1cnc2c(N)ncnn12] |
| Example 127F | 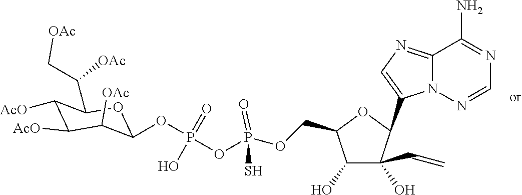 [SMILES: C=C[C@@]1(O)[C@H](O)[C@@H](CO[P@](=O)(S)OP(=O)(O)OC2OC([C@H](COC(C)=O)OC(C)=O)C(OC(C)=O)C(OC(C)=O)C2OC(C)=O)O[C@H]1c1cnc2c(N)ncnn12] |
| | 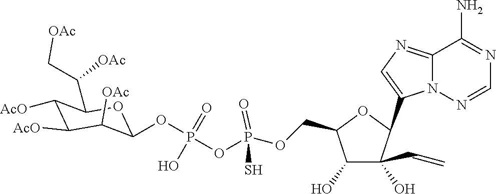 [SMILES: C=C[C@@]1(O)[C@H](O)[C@@H](CO[P@](=O)(S)OP(=O)(O)OC2OC([C@H](COC(C)=O)OC(C)=O)C(OC(C)=O)C(OC(C)=O)C2OC(C)=O)O[C@H]1c1cnc2c(N)ncnn12] |
| Example 127G | 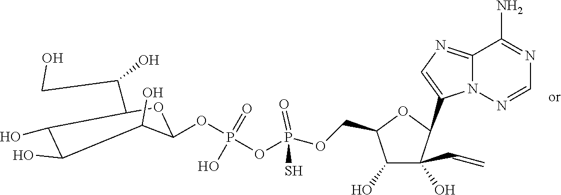 [SMILES: C=C[C@@]1(O)[C@H](O)[C@@H](CO[P@](=O)(S)OP(=O)(O)OC2OC([C@@H](O)CO)C(O)C(O)C2O)O[C@H]1c1cnc2c(N)ncnn12] |
| | 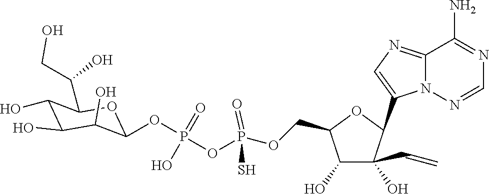 [SMILES: C=C[C@@]1(O)[C@H](O)[C@@H](CO[P@](=O)(S)OP(=O)(O)OC2OC([C@@H](O)CO)C(O)C(O)C2O)O[C@H]1c1cnc2c(N)ncnn12] |
| Example 127H | 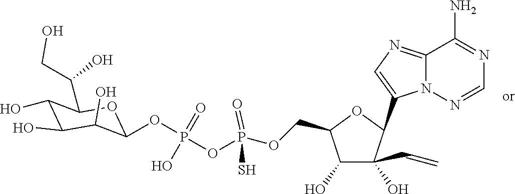 [SMILES: C=C[C@@]1(O)[C@H](O)[C@@H](CO[P@](=O)(S)OP(=O)(O)OC2OC([C@@H](O)CO)C(O)C(O)C2O)O[C@H]1c1cnc2c(N)ncnn12] |
| | 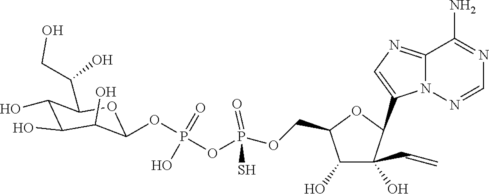 [SMILES: C=C[C@@]1(O)[C@H](O)[C@@H](CO[P@](=O)(S)OP(=O)(O)OC2OC([C@@H](O)CO)C(O)C(O)C2O)O[C@H]1c1cnc2c(N)ncnn12] |
| Example 128A | 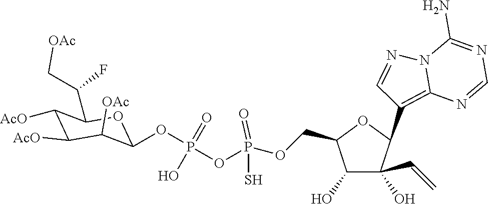 [SMILES: C=C[C@@]1(O)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1cnn2c(N)ncnc12] |
| Example 128B | 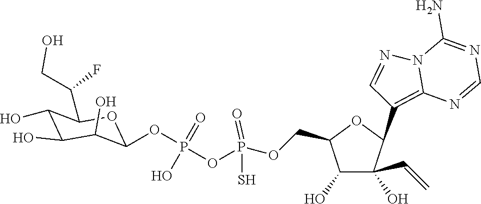 [SMILES: C=C[C@@]1(O)[C@H](O)[C@@H](COP(=O)(S)OP(=O)(O)OC2OC([C@@H](F)CO)C(O)C(O)C2O)O[C@H]1c1cnn2c(N)ncnc12] |
| Example 128C | 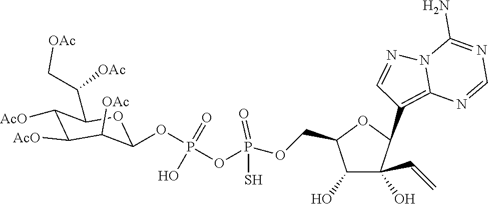 [SMILES: C=C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1cnn2c(N)ncnc12] |
| Example 128D | 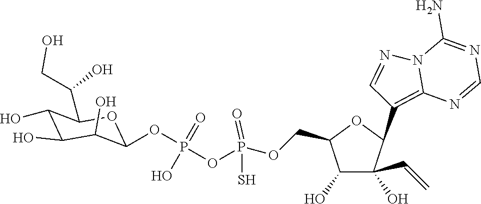 [SMILES: C=C[C@@]1(O)[C@H](O)[C@@H](COP(=O)(S)OP(=O)(O)OC2OC([C@@H](O)CO)C(O)C(O)C2O)O[C@H]1c1cnn2c(N)ncnc12] |
| Example 133A | 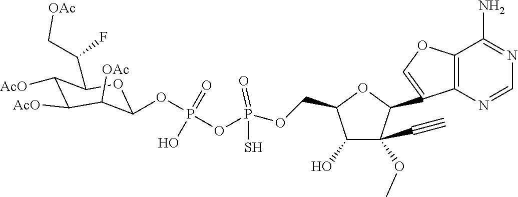 [SMILES: C#C[C@@]1(OC)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1coc2c(N)ncnc12] |
| Example 133B | 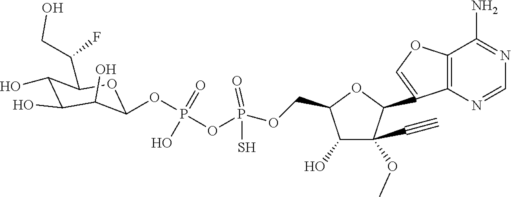 [SMILES: C#C[C@@]1(OC)[C@H](O)[C@@H](COP(=O)(S)OP(=O)(O)OC2OC([C@@H](F)CO)C(O)C(O)C2O)O[C@H]1c1coc2c(N)ncnc12] |
| Example 133C | 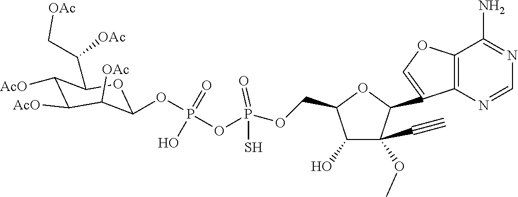 [SMILES: C#C[C@@]1(OC)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1coc2c(N)ncnc12] |
| Example 133D | 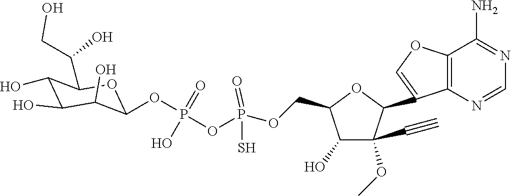 [SMILES: C#C[C@@]1(OC)[C@H](O)[C@@H](COP(=O)(S)OP(=O)(O)OC2OC([C@@H](O)CO)C(O)C(O)C2O)O[C@H]1c1coc2c(N)ncnc12] |
| Example 134A | 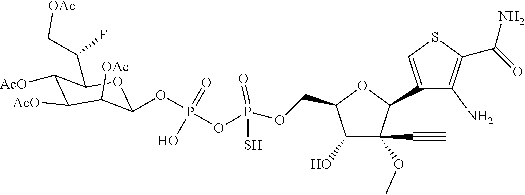 [SMILES: C#C[C@@]1(OC)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1csc(C(N)=O)c1N] |
| Example 134B | 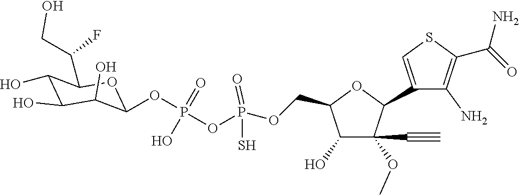 [SMILES: C#C[C@@]1(OC)[C@H](O)[C@@H](COP(=O)(S)OP(=O)(O)OC2OC([C@@H](F)CO)C(O)C(O)C2O)O[C@H]1c1csc(C(N)=O)c1N] |
| Example 134C | 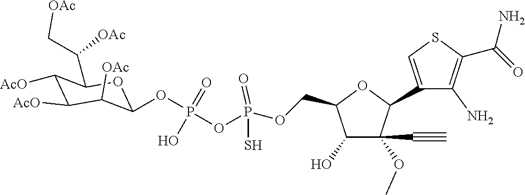 [SMILES: C#C[C@@]1(OC)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1csc(C(N)=O)c1N] |
| Example 134D | 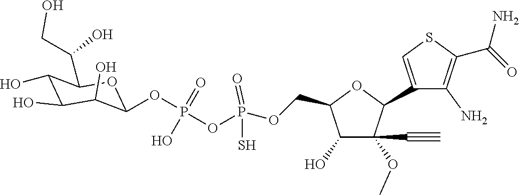 [SMILES: C#C[C@@]1(OC)[C@H](O)[C@@H](COP(=O)(S)OP(=O)(O)OC2OC([C@@H](O)CO)C(O)C(O)C2O)O[C@H]1c1csc(C(N)=O)c1N] |
| Example 135A | 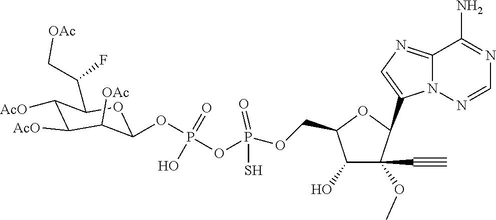 [SMILES: C#C[C@@]1(OC)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1cnc2c(N)ncnn12] |
| Example 135B | 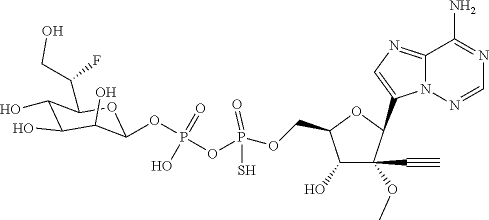 [SMILES: C#C[C@@]1(OC)[C@H](O)[C@@H](COP(=O)(S)OP(=O)(O)OC2OC([C@@H](F)CO)C(O)C(O)C2O)O[C@H]1c1cnc2c(N)ncnn12] |
| Example 135C | 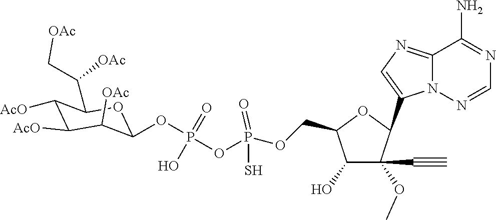 [SMILES: C#C[C@@]1(OC)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1cnc2c(N)ncnn12] |
| Example 135D | 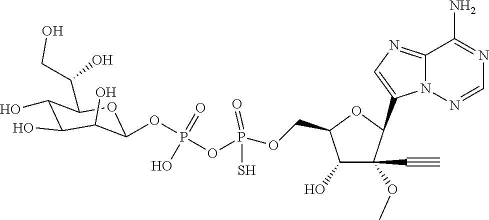 [SMILES: C#C[C@@]1(OC)[C@H](O)[C@@H](COP(=O)(S)OP(=O)(O)OC2OC([C@@H](O)CO)C(O)C(O)C2O)O[C@H]1c1cnc2c(N)ncnn12] |
| Example 136A | 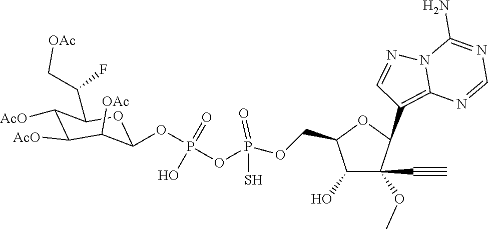 [SMILES: C#C[C@@]1(OC)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1cnn2c(N)ncnc12] |
| Example 136B | 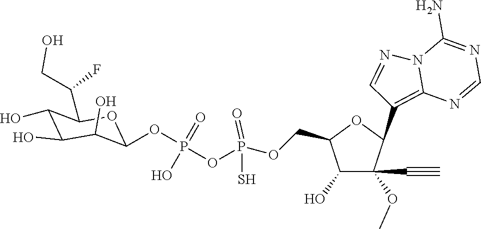 [SMILES: C#C[C@@]1(OC)[C@H](O)[C@@H](COP(=O)(S)OP(=O)(O)OC2OC([C@@H](F)CO)C(O)C(O)C2O)O[C@H]1c1cnn2c(N)ncnc12] |
| Example 136C | 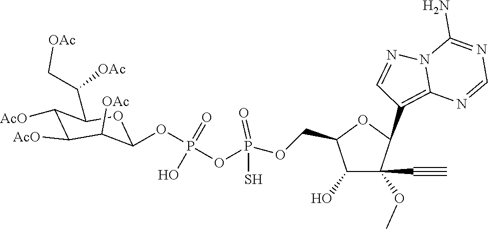 [SMILES: C#C[C@@]1(OC)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1cnn2c(N)ncnc12] |
| Example 136D | 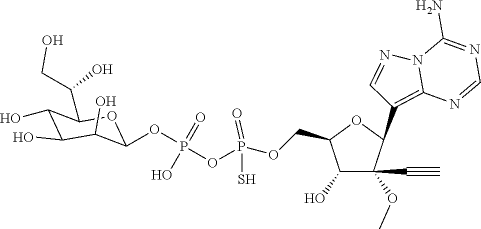 [SMILES: C#C[C@@]1(OC)[C@H](O)[C@@H](COP(=O)(S)OP(=O)(O)OC2OC([C@@H](O)CO)C(O)C(O)C2O)O[C@H]1c1cnn2c(N)ncnc12] |
| Example 137A | 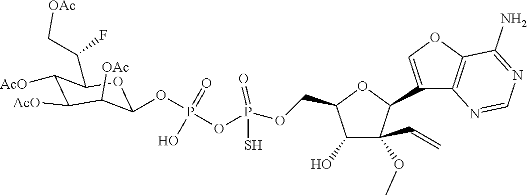 [SMILES: C=C[C@@]1(OC)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1coc2c(N)ncnc12] |
| Example 137B | 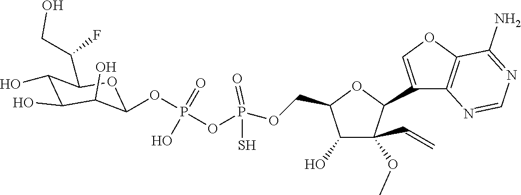 [SMILES: C=C[C@@]1(OC)[C@H](O)[C@@H](COP(=O)(S)OP(=O)(O)OC2OC([C@@H](F)CO)C(O)C(O)C2O)O[C@H]1c1coc2c(N)ncnc12] |
| Example 137C | 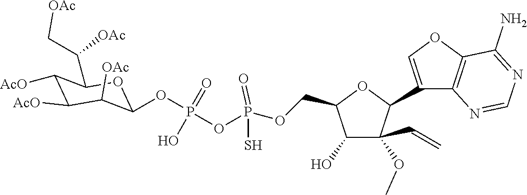 [SMILES: C=C[C@@]1(OC)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1coc2c(N)ncnc12] |
| Example 137D | 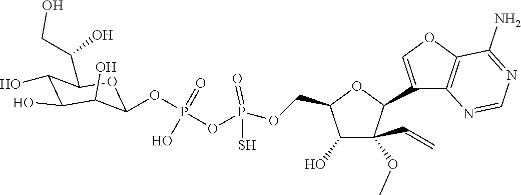 [SMILES: C=C[C@@]1(OC)[C@H](O)[C@@H](COP(=O)(S)OP(=O)(O)OC2OC([C@@H](O)CO)C(O)C(O)C2O)O[C@H]1c1coc2c(N)ncnc12] |
| Example 138A | 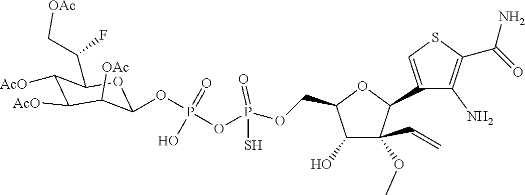 [SMILES: C=C[C@@]1(OC)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1csc(C(N)=O)c1N] |
| Example 138B | 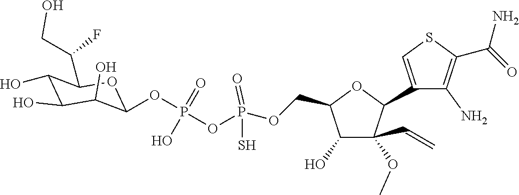 [SMILES: C=C[C@@]1(OC)[C@H](O)[C@@H](COP(=O)(S)OP(=O)(O)OC2OC([C@@H](F)CO)C(O)C(O)C2O)O[C@H]1c1csc(C(N)=O)c1N] |
| Example 138C | 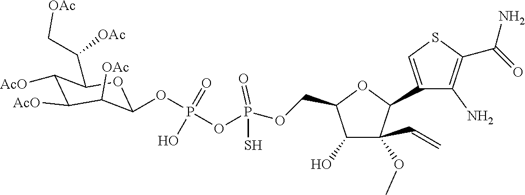 [SMILES: C=C[C@@]1(OC)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1csc(C(N)=O)c1N] |
| Example 138D | 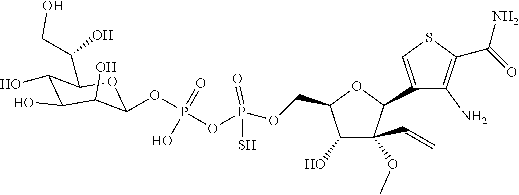 [SMILES: C=C[C@@]1(OC)[C@H](O)[C@@H](COP(=O)(S)OP(=O)(O)OC2OC([C@@H](O)CO)C(O)C(O)C2O)O[C@H]1c1csc(C(N)=O)c1N] |
| Example 139A | 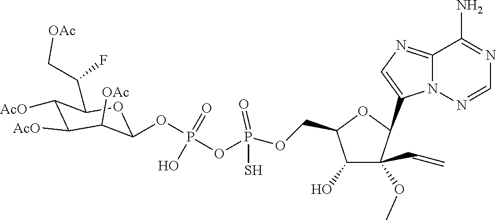 [SMILES: C=C[C@@]1(OC)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1cnc2c(N)ncnn12] |
| Example 139B | 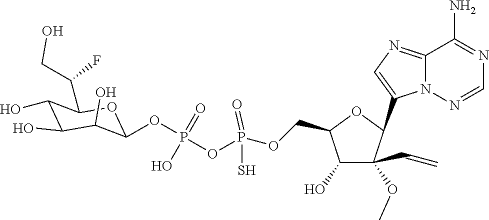 [SMILES: C=C[C@@]1(OC)[C@H](O)[C@@H](COP(=O)(S)OP(=O)(O)OC2OC([C@@H](F)CO)C(O)C(O)C2O)O[C@H]1c1cnc2c(N)ncnn12] |
| Example 139C | 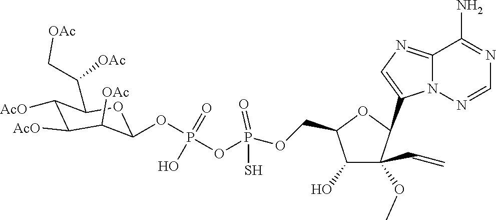 [SMILES: C=C[C@@]1(OC)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1cnc2c(N)ncnn12] |
| Example 139D | 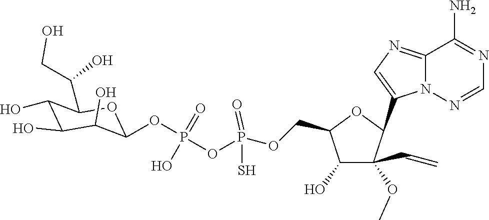 [SMILES: C=C[C@@]1(OC)[C@H](O)[C@@H](COP(=O)(S)OP(=O)(O)OC2OC([C@@H](O)CO)C(O)C(O)C2O)O[C@H]1c1cnc2c(N)ncnn12] |
| Example 140A | 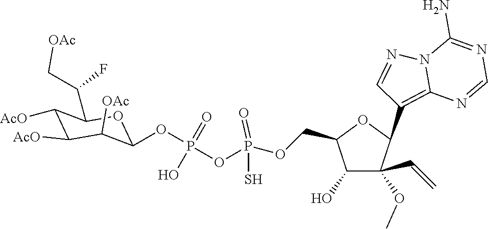 [SMILES: C=C[C@@]1(OC)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1cnn2c(N)ncnc12] |
| Example 140B | 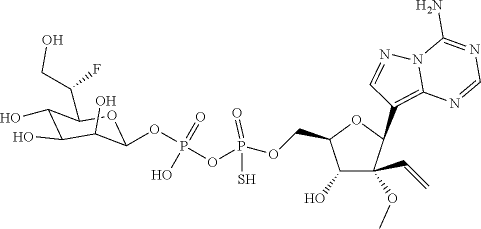 [SMILES: C=C[C@@]1(OC)[C@H](O)[C@@H](COP(=O)(S)OP(=O)(O)OC2OC([C@@H](F)CO)C(O)C(O)C2O)O[C@H]1c1cnn2c(N)ncnc12] |
| Example 140C | 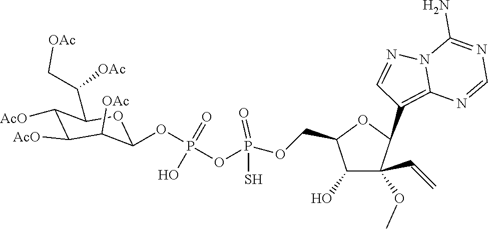 [SMILES: C=C[C@@]1(OC)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1cnn2c(N)ncnc12] |
| Example 140D | 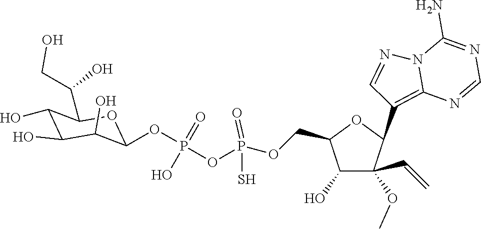 [SMILES: C=C[C@@]1(OC)[C@H](O)[C@@H](COP(=O)(S)OP(=O)(O)OC2OC([C@@H](O)CO)C(O)C(O)C2O)O[C@H]1c1cnn2c(N)ncnc12] |
| Example 141A | 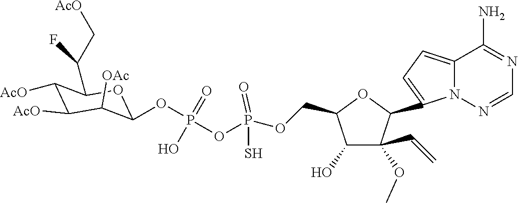 [SMILES: C=C[C@@]1(OC)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1ccc2c(N)ncnn12] |
| Example 141B | 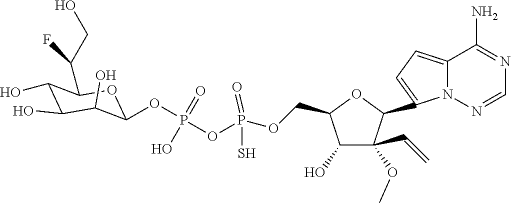 [SMILES: C=C[C@@]1(OC)[C@H](O)[C@@H](COP(=O)(S)OP(=O)(O)OC2OC([C@@H](F)CO)C(O)C(O)C2O)O[C@H]1c1ccc2c(N)ncnn12] |
| Example 141C | 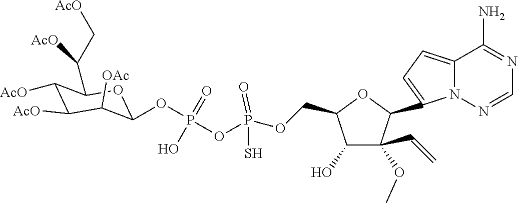 [SMILES: C=C[C@@]1(OC)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1ccc2c(N)ncnn12] |
| Example 141D | 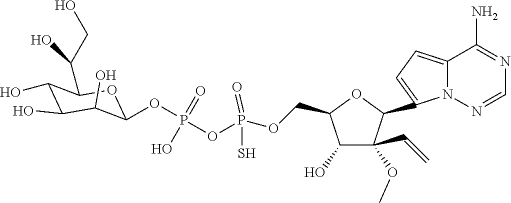 [SMILES: C=C[C@@]1(OC)[C@H](O)[C@@H](COP(=O)(S)OP(=O)(O)OC2OC([C@@H](O)CO)C(O)C(O)C2O)O[C@H]1c1ccc2c(N)ncnn12] |
| Example 142A | 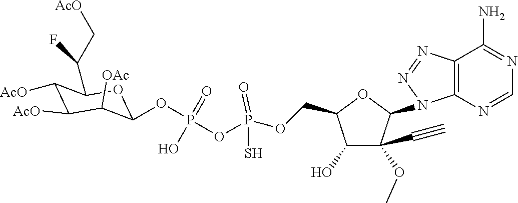 [SMILES: C#C[C@@]1(OC)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1nnc2c(N)ncnc21] |
| Example 142B | 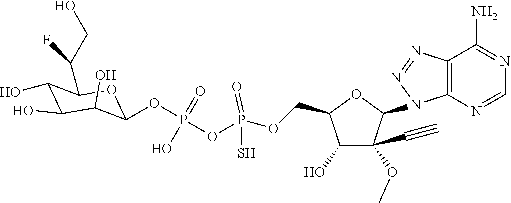 [SMILES: C#C[C@@]1(OC)[C@H](O)[C@@H](COP(=O)(S)OP(=O)(O)OC2OC([C@@H](F)CO)C(O)C(O)C2O)O[C@H]1n1nnc2c(N)ncnc21] |
| Example 142C | 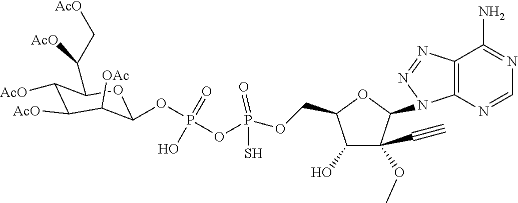 [SMILES: C#C[C@@]1(OC)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1nnc2c(N)ncnc21] |
| Example 142D | 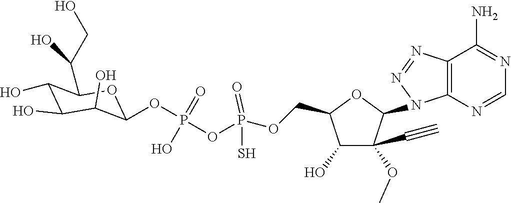 [SMILES: C#C[C@@]1(OC)[C@H](O)[C@@H](COP(=O)(S)OP(=O)(O)OC2OC([C@@H](O)CO)C(O)C(O)C2O)O[C@H]1n1nnc2c(N)ncnc21] |
| Example 143A | 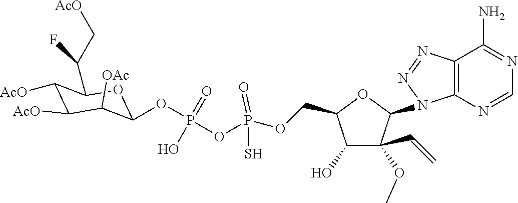 [SMILES: C=C[C@@]1(OC)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1nnc2c(N)ncnc21] |
| Example 143B | 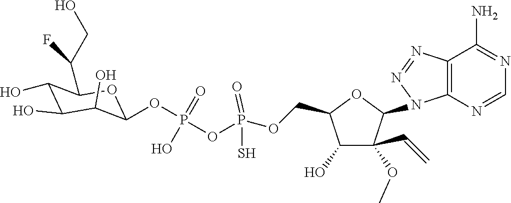 [SMILES: C=C[C@@]1(OC)[C@H](O)[C@@H](COP(=O)(S)OP(=O)(O)OC2OC([C@@H](F)CO)C(O)C(O)C2O)O[C@H]1n1nnc2c(N)ncnc21] |
| Example 143C | 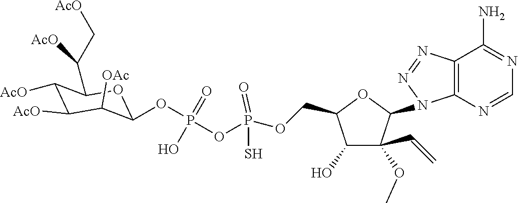 [SMILES: C=C[C@@]1(OC)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1nnc2c(N)ncnc21] |
| Example 143D | 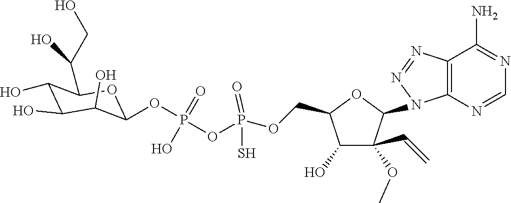 [SMILES: C=C[C@@]1(OC)[C@H](O)[C@@H](COP(=O)(S)OP(=O)(O)OC2OC([C@@H](O)CO)C(O)C(O)C2O)O[C@H]1n1nnc2c(N)ncnc21] |
| Example 162A | 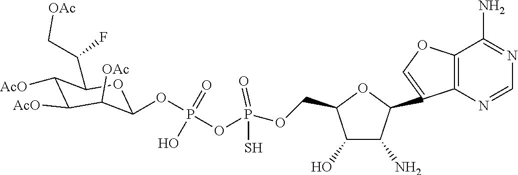 [SMILES: CC(=O)OC[C@H](F)C1OC(OP(=O)(O)OP(=O)(S)OC[C@H]2O[C@@H](c3coc4c(N)ncnc34)[C@H](N)[C@@H]2O)C(OC(C)=O)C(OC(C)=O)C1OC(C)=O] |
| Example 162B | 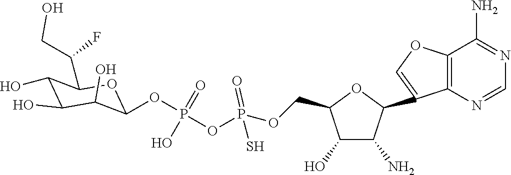 [SMILES: Nc1ncnc2c([C@@H]3O[C@H](COP(=O)(S)OP(=O)(O)OC4OC([C@@H](F)CO)C(O)C(O)C4O)[C@@H](O)[C@H]3N)coc12] |
| Example 162C | 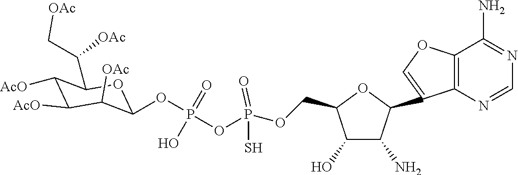 [SMILES: CC(=O)OC[C@H](OC(C)=O)C1OC(OP(=O)(O)OP(=O)(S)OC[C@H]2O[C@@H](c3coc4c(N)ncnc34)[C@H](N)[C@@H]2O)C(OC(C)=O)C(OC(C)=O)C1OC(C)=O] |
| Example 162D | 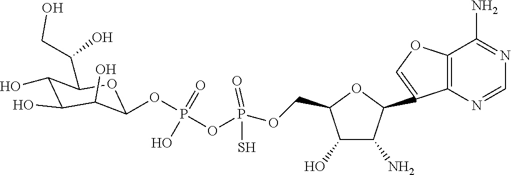 [SMILES: Nc1ncnc2c([C@@H]3O[C@H](COP(=O)(S)OP(=O)(O)OC4OC([C@@H](O)CO)C(O)C(O)C4O)[C@@H](O)[C@H]3N)coc12] |
| Example 164A | 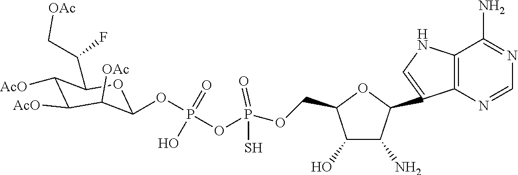 [SMILES: CC(=O)OC[C@H](F)C1OC(OP(=O)(O)OP(=O)(S)OC[C@H]2O[C@@H](c3c[nH]c4c(N)ncnc34)[C@H](N)[C@@H]2O)C(OC(C)=O)C(OC(C)=O)C1OC(C)=O] |
| Example 164B | 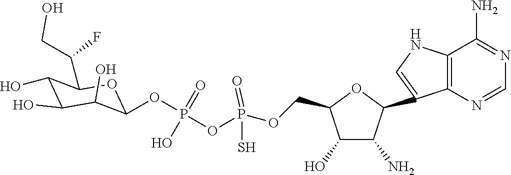 [SMILES: Nc1ncnc2c([C@@H]3O[C@H](COP(=O)(S)OP(=O)(O)OC4OC([C@@H](F)CO)C(O)C(O)C4O)[C@@H](O)[C@H]3N)c[nH]c12] |
| Example 164C | 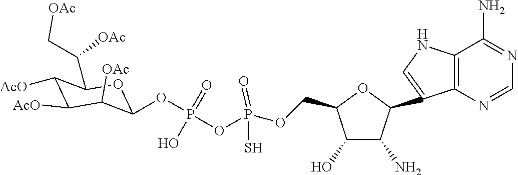 [SMILES: CC(=O)OC[C@H](OC(C)=O)C1OC(OP(=O)(O)OP(=O)(S)OC[C@H]2O[C@@H](c3c[nH]c4c(N)ncnc34)[C@H](N)[C@@H]2O)C(OC(C)=O)C(OC(C)=O)C1OC(C)=O] |
| Example 164D | 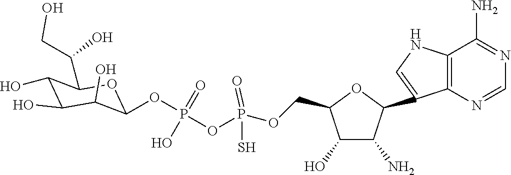 [SMILES: Nc1ncnc2c([C@@H]3O[C@H](COP(=O)(S)OP(=O)(O)OC4OC([C@@H](O)CO)C(O)C(O)C4O)[C@@H](O)[C@H]3N)c[nH]c12] |
| Example 166A | 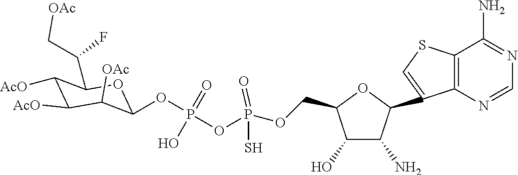 [SMILES: CC(=O)OC[C@H](F)C1OC(OP(=O)(O)OP(=O)(S)OC[C@H]2O[C@@H](c3csc4c(N)ncnc34)[C@H](N)[C@@H]2O)C(OC(C)=O)C(OC(C)=O)C1OC(C)=O] |
| Example 166B | 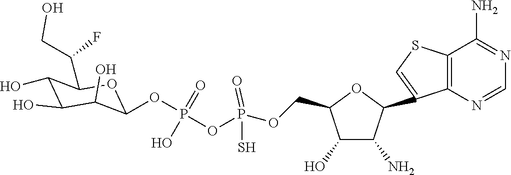 [SMILES: Nc1ncnc2c([C@@H]3O[C@H](COP(=O)(S)OP(=O)(O)OC4OC([C@@H](F)CO)C(O)C(O)C4O)[C@@H](O)[C@H]3N)csc12] |
| Example 166C | 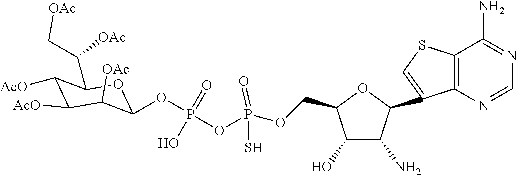 [SMILES: CC(=O)OC[C@H](OC(C)=O)C1OC(OP(=O)(O)OP(=O)(S)OC[C@H]2O[C@@H](c3csc4c(N)ncnc34)[C@H](N)[C@@H]2O)C(OC(C)=O)C(OC(C)=O)C1OC(C)=O] |
| Example 166D | 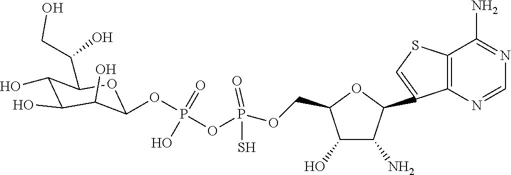 [SMILES: Nc1ncnc2c([C@@H]3O[C@H](COP(=O)(S)OP(=O)(O)OC4OC([C@@H](O)CO)C(O)C(O)C4O)[C@@H](O)[C@H]3N)csc12] |
| Example 167A | 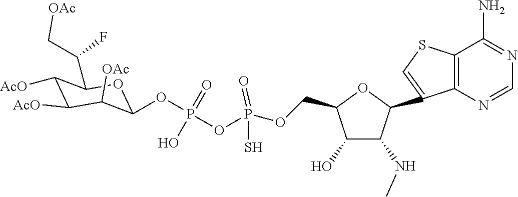 [SMILES: CN[C@@H]1[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1csc2c(N)ncnc12] |
| Example 167B | 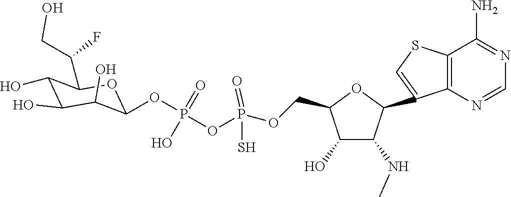 [SMILES: CN[C@@H]1[C@H](O)[C@@H](COP(=O)(S)OP(=O)(O)OC2OC([C@@H](F)CO)C(O)C(O)C2O)O[C@H]1c1csc2c(N)ncnc12] |
| Example 167C | 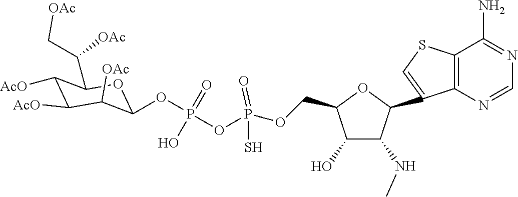 [SMILES: CN[C@@H]1[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1csc2c(N)ncnc12] |
| Example 167D | 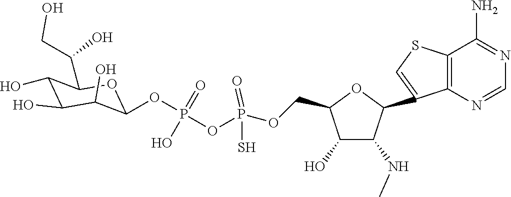 [SMILES: CN[C@@H]1[C@H](O)[C@@H](COP(=O)(S)OP(=O)(O)OC2OC([C@@H](O)CO)C(O)C(O)C2O)O[C@H]1c1csc2c(N)ncnc12] |
| Example 168A | 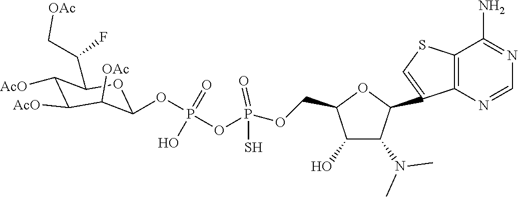 [SMILES: CC(=O)OC[C@H](F)C1OC(OP(=O)(O)OP(=O)(S)OC[C@H]2O[C@@H](c3csc4c(N)ncnc34)[C@H](N(C)C)[C@@H]2O)C(OC(C)=O)C(OC(C)=O)C1OC(C)=O] |
| Example 168B | 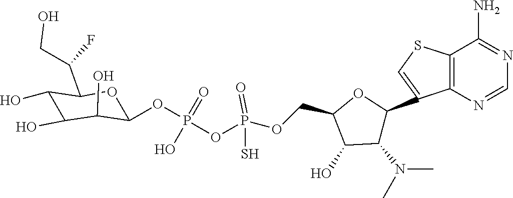 [SMILES: CN(C)[C@@H]1[C@H](O)[C@@H](COP(=O)(S)OP(=O)(O)OC2OC([C@@H](F)CO)C(O)C(O)C2O)O[C@H]1c1csc2c(N)ncnc12] |
| Example 168C | 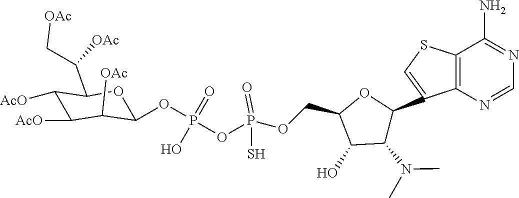 [SMILES: CC(=O)OC[C@H](OC(C)=O)C1OC(OP(=O)(O)OP(=O)(S)OC[C@H]2O[C@@H](c3csc4c(N)ncnc34)[C@H](N(C)C)[C@@H]2O)C(OC(C)=O)C(OC(C)=O)C1OC(C)=O] |
| Example 168D | 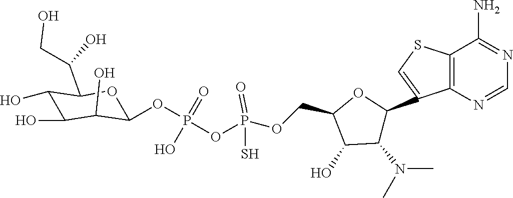 [SMILES: CN(C)[C@@H]1[C@H](O)[C@@H](COP(=O)(S)OP(=O)(O)OC2OC([C@@H](O)CO)C(O)C(O)C2O)O[C@H]1c1csc2c(N)ncnc12] |
| Example 169A | 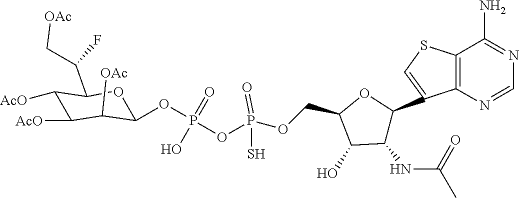 [SMILES: CC(=O)N[C@@H]1[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1csc2c(N)ncnc12] |
| Example 169B | 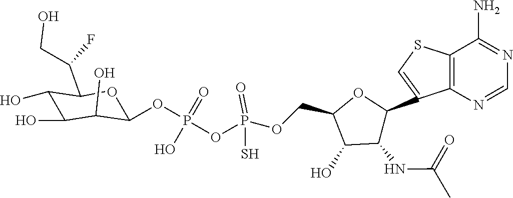 [SMILES: CC(=O)N[C@@H]1[C@H](O)[C@@H](COP(=O)(S)OP(=O)(O)OC2OC([C@@H](F)CO)C(O)C(O)C2O)O[C@H]1c1csc2c(N)ncnc12] |
| Example 169C | 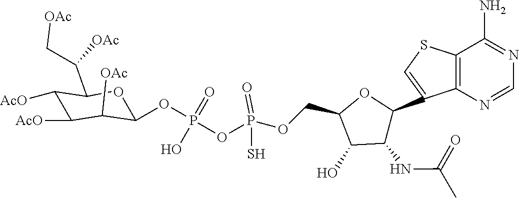 [SMILES: CC(=O)N[C@@H]1[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1csc2c(N)ncnc12] |
| Example 169D | 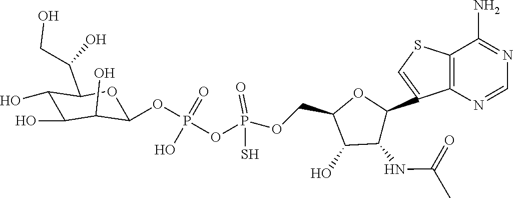 [SMILES: CC(=O)N[C@@H]1[C@H](O)[C@@H](COP(=O)(S)OP(=O)(O)OC2OC([C@@H](O)CO)C(O)C(O)C2O)O[C@H]1c1csc2c(N)ncnc12] |
| Example 170A | 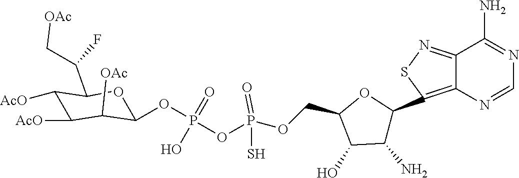 [SMILES: CC(=O)OC[C@H](F)C1OC(OP(=O)(O)OP(=O)(S)OC[C@H]2O[C@@H](c3snc4c(N)ncnc34)[C@H](N)[C@@H]2O)C(OC(C)=O)C(OC(C)=O)C1OC(C)=O] |
| Example 170B | 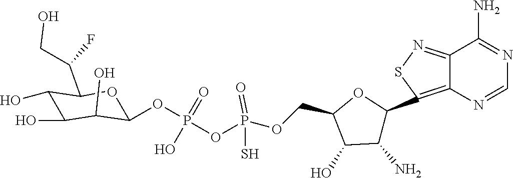 [SMILES: Nc1ncnc2c([C@@H]3O[C@H](COP(=O)(S)OP(=O)(O)OC4OC([C@@H](F)CO)C(O)C(O)C4O)[C@@H](O)[C@H]3N)snc12] |
| Example 170C | 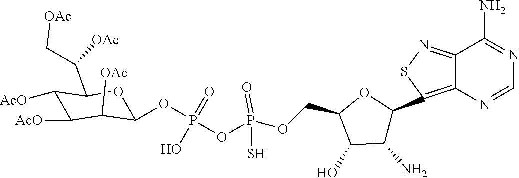 [SMILES: CC(=O)OC[C@H](OC(C)=O)C1OC(OP(=O)(O)OP(=O)(S)OC[C@H]2O[C@@H](c3snc4c(N)ncnc34)[C@H](N)[C@@H]2O)C(OC(C)=O)C(OC(C)=O)C1OC(C)=O] |
| Example 170D | 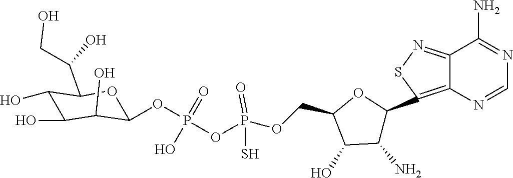 [SMILES: Nc1ncnc2c([C@@H]3O[C@H](COP(=O)(S)OP(=O)(O)OC4OC([C@@H](O)CO)C(O)C(O)C4O)[C@@H](O)[C@H]3N)snc12] |
| Example 171A | 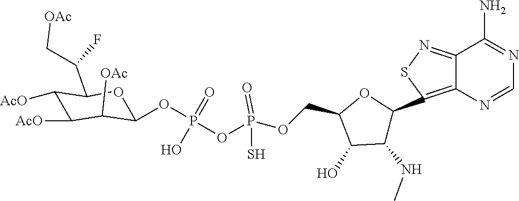 [SMILES: CN[C@@H]1[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1snc2c(N)ncnc12] |
| Example 171B | 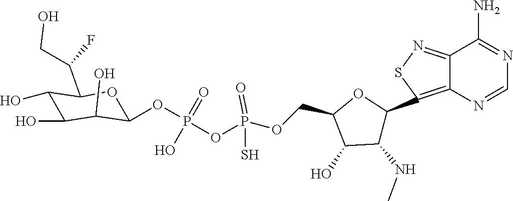 [SMILES: CN[C@@H]1[C@H](O)[C@@H](COP(=O)(S)OP(=O)(O)OC2OC([C@@H](F)CO)C(O)C(O)C2O)O[C@H]1c1snc2c(N)ncnc12] |
| Example 171C | 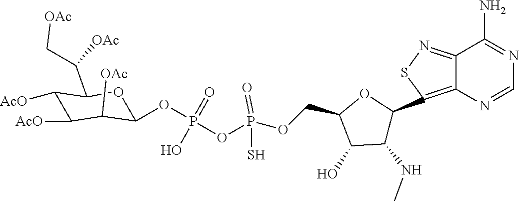 [SMILES: CN[C@@H]1[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1snc2c(N)ncnc12] |
| Example 171D | 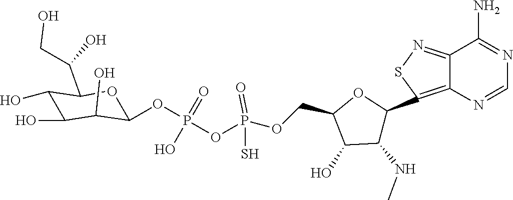 [SMILES: CN[C@@H]1[C@H](O)[C@@H](COP(=O)(S)OP(=O)(O)OC2OC([C@@H](O)CO)C(O)C(O)C2O)O[C@H]1c1snc2c(N)ncnc12] |
| Example 172A | 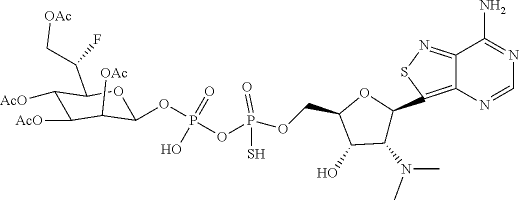 [SMILES: CC(=O)OC[C@H](F)C1OC(OP(=O)(O)OP(=O)(S)OC[C@H]2O[C@@H](c3snc4c(N)ncnc34)[C@H](N(C)C)[C@@H]2O)C(OC(C)=O)C(OC(C)=O)C1OC(C)=O] |
| Example 172B | 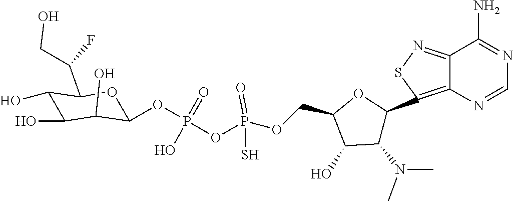 [SMILES: CN(C)[C@@H]1[C@H](O)[C@@H](COP(=O)(S)OP(=O)(O)OC2OC([C@@H](F)CO)C(O)C(O)C2O)O[C@H]1c1snc2c(N)ncnc12] |
| Example 172C | 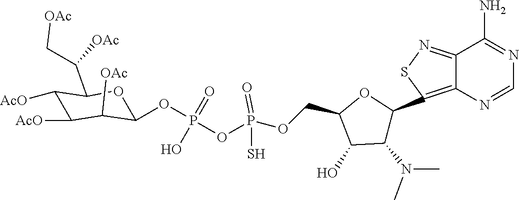 [SMILES: CC(=O)OC[C@H](OC(C)=O)C1OC(OP(=O)(O)OP(=O)(S)OC[C@H]2O[C@@H](c3snc4c(N)ncnc34)[C@H](N(C)C)[C@@H]2O)C(OC(C)=O)C(OC(C)=O)C1OC(C)=O] |
| Example 172D | 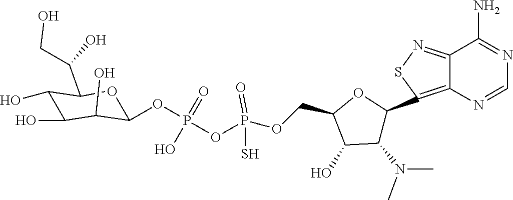 [SMILES: CN(C)[C@@H]1[C@H](O)[C@@H](COP(=O)(S)OP(=O)(O)OC2OC([C@@H](O)CO)C(O)C(O)C2O)O[C@H]1c1snc2c(N)ncnc12] |
| Example 173A | 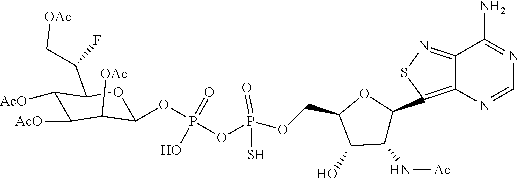 [SMILES: CC(=O)N[C@@H]1[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1snc2c(N)ncnc12] |
| Example 173B | 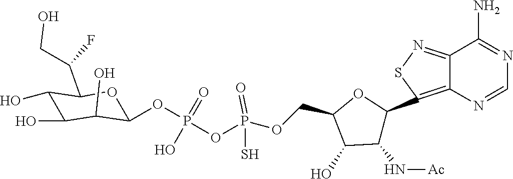 [SMILES: CC(=O)N[C@@H]1[C@H](O)[C@@H](COP(=O)(S)OP(=O)(O)OC2OC([C@@H](F)CO)C(O)C(O)C2O)O[C@H]1c1snc2c(N)ncnc12] |
| Example 173C | 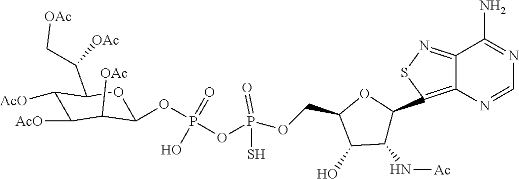 [SMILES: CC(=O)N[C@@H]1[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1snc2c(N)ncnc12] |
| Example 173D | 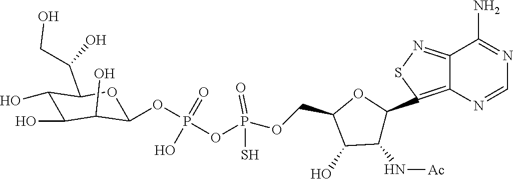 [SMILES: CC(=O)N[C@@H]1[C@H](O)[C@@H](COP(=O)(S)OP(=O)(O)OC2OC([C@@H](O)CO)C(O)C(O)C2O)O[C@H]1c1snc2c(N)ncnc12] |
| Example 174A | 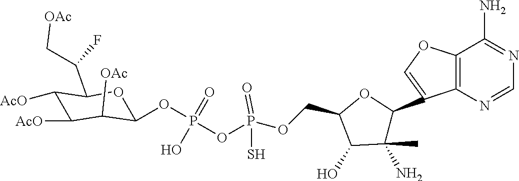 [SMILES: CC(=O)OC[C@H](F)C1OC(OP(=O)(O)OP(=O)(S)OC[C@H]2O[C@@H](c3coc4c(N)ncnc34)[C@](C)(N)[C@@H]2O)C(OC(C)=O)C(OC(C)=O)C1OC(C)=O] |
| Example 174B | 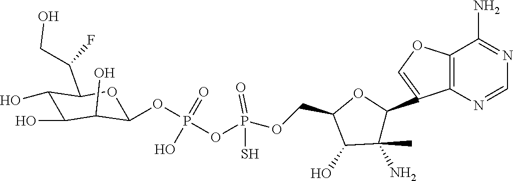 [SMILES: C[C@@]1(N)[C@H](O)[C@@H](COP(=O)(S)OP(=O)(O)OC2OC([C@@H](F)CO)C(O)C(O)C2O)O[C@H]1c1coc2c(N)ncnc12] |
| Example 174C | 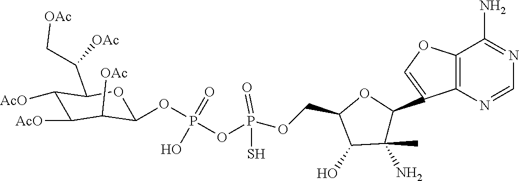 [SMILES: CC(=O)OC[C@H](OC(C)=O)C1OC(OP(=O)(O)OP(=O)(S)OC[C@H]2O[C@@H](c3coc4c(N)ncnc34)[C@](C)(N)[C@@H]2O)C(OC(C)=O)C(OC(C)=O)C1OC(C)=O] |
| Example 174D | 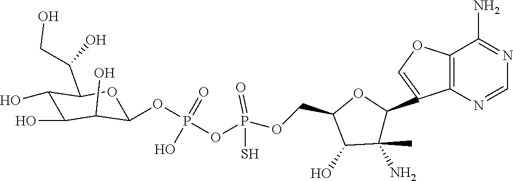 [SMILES: C[C@@]1(N)[C@H](O)[C@@H](COP(=O)(S)OP(=O)(O)OC2OC([C@@H](O)CO)C(O)C(O)C2O)O[C@H]1c1coc2c(N)ncnc12] |
| Example 175A | 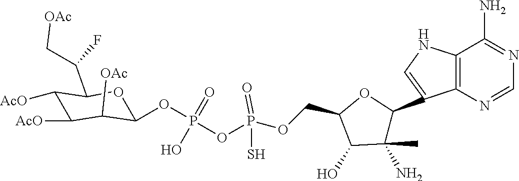 [SMILES: CC(=O)OC[C@H](F)C1OC(OP(=O)(O)OP(=O)(S)OC[C@H]2O[C@@H](c3c[nH]c4c(N)ncnc34)[C@](C)(N)[C@@H]2O)C(OC(C)=O)C(OC(C)=O)C1OC(C)=O] |
| Example 175B | 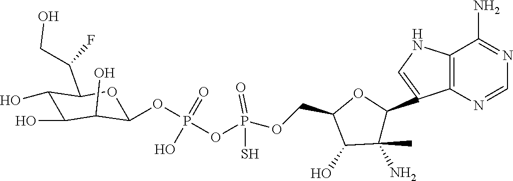 [SMILES: C[C@@]1(N)[C@H](O)[C@@H](COP(=O)(S)OP(=O)(O)OC2OC([C@@H](F)CO)C(O)C(O)C2O)O[C@H]1c1c[nH]c2c(N)ncnc12] |
| Example 175C | 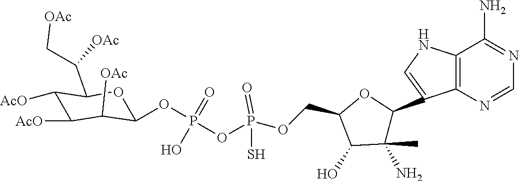 [SMILES: CC(=O)OC[C@H](OC(C)=O)C1OC(OP(=O)(O)OP(=O)(S)OC[C@H]2O[C@@H](c3c[nH]c4c(N)ncnc34)[C@](C)(N)[C@@H]2O)C(OC(C)=O)C(OC(C)=O)C1OC(C)=O] |
| Example 175D | 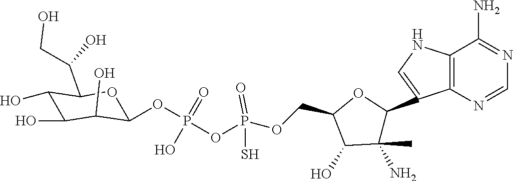 [SMILES: C[C@@]1(N)[C@H](O)[C@@H](COP(=O)(S)OP(=O)(O)OC2OC([C@@H](O)CO)C(O)C(O)C2O)O[C@H]1c1c[nH]c2c(N)ncnc12] |
| Example 176A | 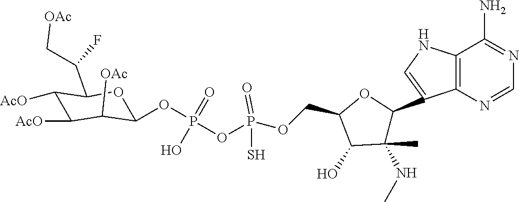 [SMILES: CN[C@]1(C)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1c[nH]c2c(N)ncnc12] |
| Example 176B | 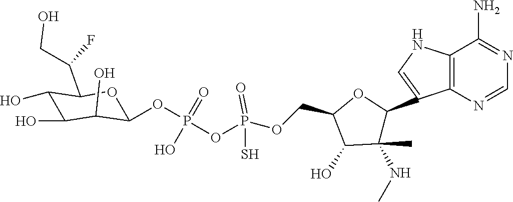 [SMILES: CN[C@]1(C)[C@H](O)[C@@H](COP(=O)(S)OP(=O)(O)OC2OC([C@@H](F)CO)C(O)C(O)C2O)O[C@H]1c1c[nH]c2c(N)ncnc12] |
| Example 176C | 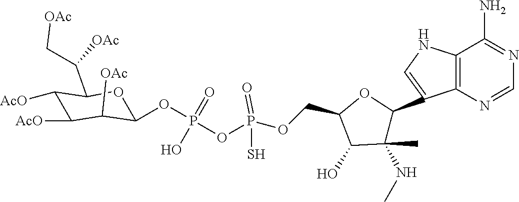 [SMILES: CN[C@]1(C)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1c[nH]c2c(N)ncnc12] |
| Example 176D | 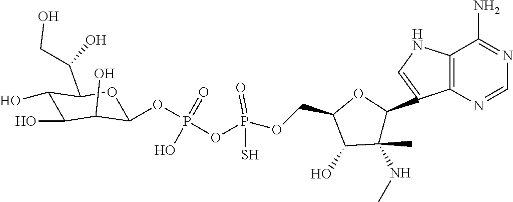 [SMILES: CN[C@]1(C)[C@H](O)[C@@H](COP(=O)(S)OP(=O)(O)OC2OC([C@@H](O)CO)C(O)C(O)C2O)O[C@H]1c1c[nH]c2c(N)ncnc12] |
| Example 177A | 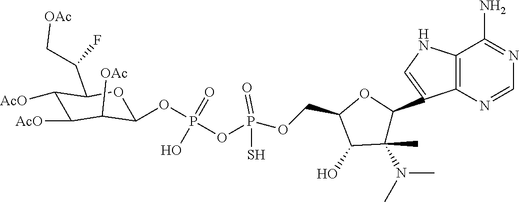 [SMILES: CC(=O)OC[C@H](F)C1OC(OP(=O)(O)OP(=O)(S)OC[C@H]2O[C@@H](c3c[nH]c4c(N)ncnc34)[C@](C)(N(C)C)[C@@H]2O)C(OC(C)=O)C(OC(C)=O)C1OC(C)=O] |
| Example 177B | 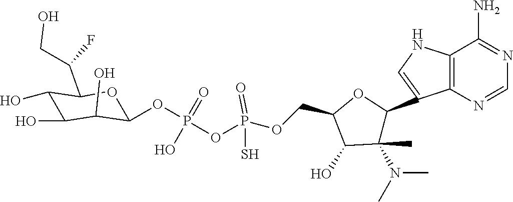 [SMILES: CN(C)[C@]1(C)[C@H](O)[C@@H](COP(=O)(S)OP(=O)(O)OC2OC([C@@H](F)CO)C(O)C(O)C2O)O[C@H]1c1c[nH]c2c(N)ncnc12] |
| Example 177C | 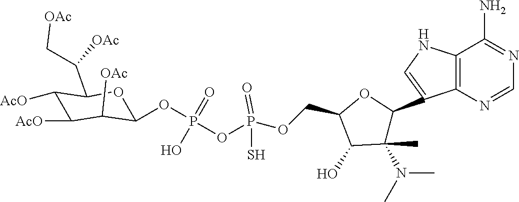 [SMILES: CC(=O)OC[C@H](OC(C)=O)C1OC(OP(=O)(O)OP(=O)(S)OC[C@H]2O[C@@H](c3c[nH]c4c(N)ncnc34)[C@](C)(N(C)C)[C@@H]2O)C(OC(C)=O)C(OC(C)=O)C1OC(C)=O] |
| Example 177D | 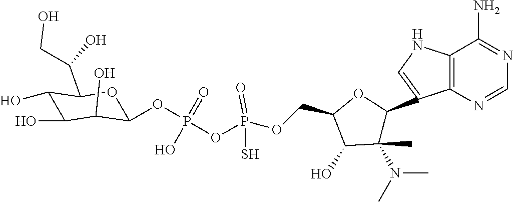 [SMILES: CN(C)[C@]1(C)[C@H](O)[C@@H](COP(=O)(S)OP(=O)(O)OC2OC([C@@H](O)CO)C(O)C(O)C2O)O[C@H]1c1c[nH]c2c(N)ncnc12] |
| Example 178A | 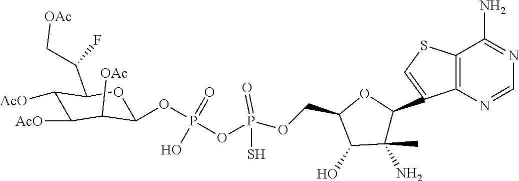 [SMILES: CC(=O)OC[C@H](F)C1OC(OP(=O)(O)OP(=O)(S)OC[C@H]2O[C@@H](c3csc4c(N)ncnc34)[C@](C)(N)[C@@H]2O)C(OC(C)=O)C(OC(C)=O)C1OC(C)=O] |
| Example 178B | 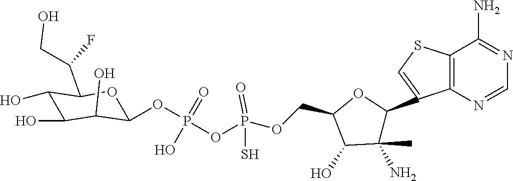 [SMILES: C[C@@]1(N)[C@H](O)[C@@H](COP(=O)(S)OP(=O)(O)OC2OC([C@@H](F)CO)C(O)C(O)C2O)O[C@H]1c1csc2c(N)ncnc12] |
| Example 178C | 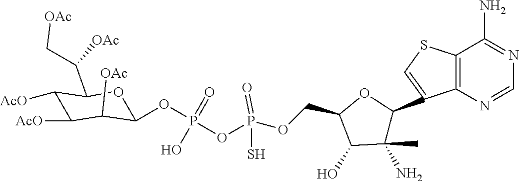 [SMILES: CC(=O)OC[C@H](OC(C)=O)C1OC(OP(=O)(O)OP(=O)(S)OC[C@H]2O[C@@H](c3csc4c(N)ncnc34)[C@](C)(N)[C@@H]2O)C(OC(C)=O)C(OC(C)=O)C1OC(C)=O] |
| Example 178D | 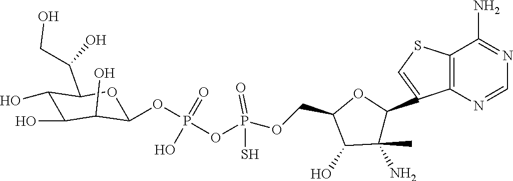 [SMILES: C[C@@]1(N)[C@H](O)[C@@H](COP(=O)(S)OP(=O)(O)OC2OC([C@@H](O)CO)C(O)C(O)C2O)O[C@H]1c1csc2c(N)ncnc12] |
| Example 179A | 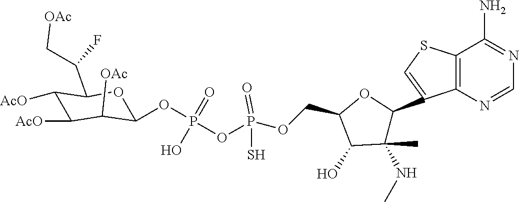 [SMILES: CN[C@]1(C)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1csc2c(N)ncnc12] |
| Example 179B | 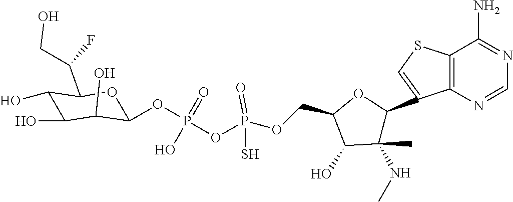 [SMILES: CN[C@]1(C)[C@H](O)[C@@H](COP(=O)(S)OP(=O)(O)OC2OC([C@@H](F)CO)C(O)C(O)C2O)O[C@H]1c1csc2c(N)ncnc12] |
| Example 179C | 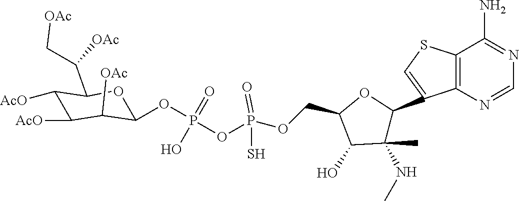 [SMILES: CN[C@]1(C)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1csc2c(N)ncnc12] |
| Example 179D | 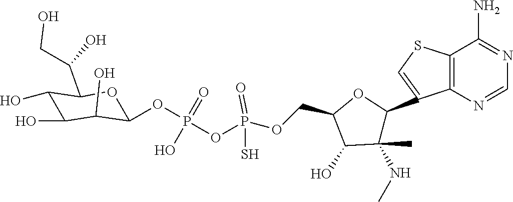 [SMILES: CN[C@]1(C)[C@H](O)[C@@H](COP(=O)(S)OP(=O)(O)OC2OC([C@@H](O)CO)C(O)C(O)C2O)O[C@H]1c1csc2c(N)ncnc12] |
| Example 180A | 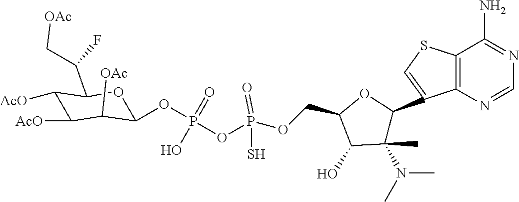 [SMILES: CC(=O)OC[C@H](F)C1OC(OP(=O)(O)OP(=O)(S)OC[C@H]2O[C@@H](c3csc4c(N)ncnc34)[C@](C)(N(C)C)[C@@H]2O)C(OC(C)=O)C(OC(C)=O)C1OC(C)=O] |
| Example 180B | 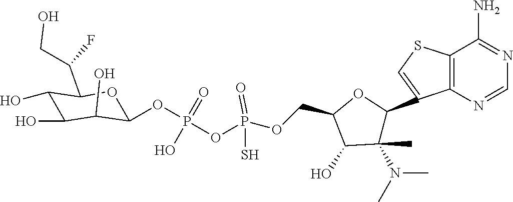 [SMILES: CN(C)[C@]1(C)[C@H](O)[C@@H](COP(=O)(S)OP(=O)(O)OC2OC([C@@H](F)CO)C(O)C(O)C2O)O[C@H]1c1csc2c(N)ncnc12] |
| Example 180C | 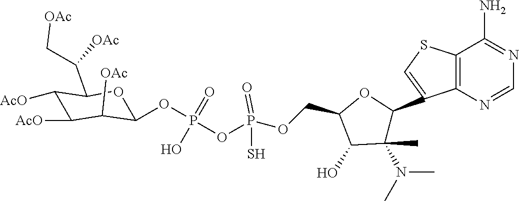 [SMILES: CC(=O)OC[C@H](OC(C)=O)C1OC(OP(=O)(O)OP(=O)(S)OC[C@H]2O[C@@H](c3csc4c(N)ncnc34)[C@](C)(N(C)C)[C@@H]2O)C(OC(C)=O)C(OC(C)=O)C1OC(C)=O] |
| Example 180D | 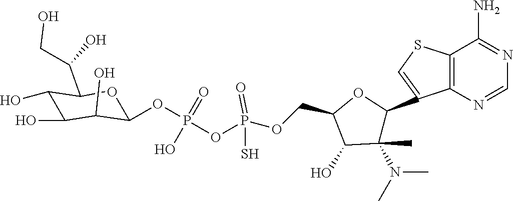 [SMILES: CN(C)[C@]1(C)[C@H](O)[C@@H](COP(=O)(S)OP(=O)(O)OC2OC([C@@H](O)CO)C(O)C(O)C2O)O[C@H]1c1csc2c(N)ncnc12] |
| Example 181A | 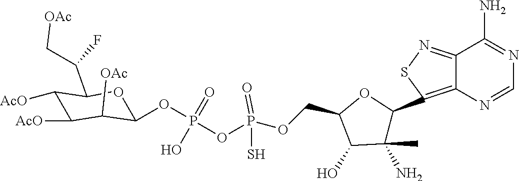 [SMILES: CC(=O)OC[C@H](F)C1OC(OP(=O)(O)OP(=O)(S)OC[C@H]2O[C@@H](c3snc4c(N)ncnc34)[C@](C)(N)[C@@H]2O)C(OC(C)=O)C(OC(C)=O)C1OC(C)=O] |
| Example 181B | 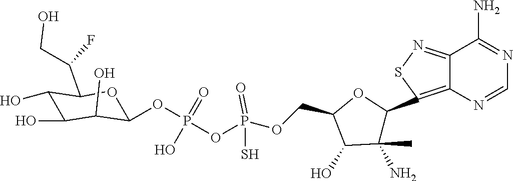 [SMILES: C[C@@]1(N)[C@H](O)[C@@H](COP(=O)(S)OP(=O)(O)OC2OC([C@@H](F)CO)C(O)C(O)C2O)O[C@H]1c1snc2c(N)ncnc12] |
| Example 181C | 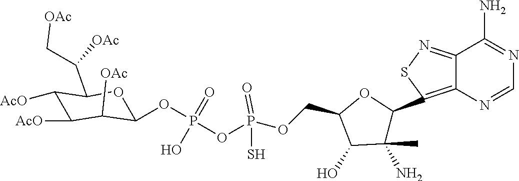 [SMILES: CC(=O)OC[C@H](OC(C)=O)C1OC(OP(=O)(O)OP(=O)(S)OC[C@H]2O[C@@H](c3snc4c(N)ncnc34)[C@](C)(N)[C@@H]2O)C(OC(C)=O)C(OC(C)=O)C1OC(C)=O] |
| Example 181D | 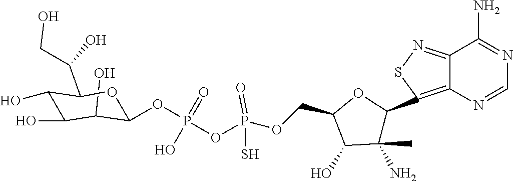 [SMILES: C[C@@]1(N)[C@H](O)[C@@H](COP(=O)(S)OP(=O)(O)OC2OC([C@@H](O)CO)C(O)C(O)C2O)O[C@H]1c1snc2c(N)ncnc12] |
| Example 182A | 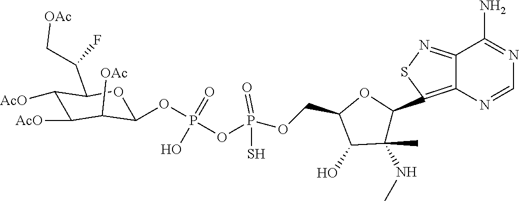 [SMILES: CN[C@]1(C)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1snc2c(N)ncnc12] |
| Example 182B | 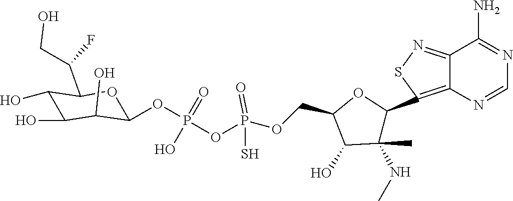 [SMILES: CN[C@]1(C)[C@H](O)[C@@H](COP(=O)(S)OP(=O)(O)OC2OC([C@@H](F)CO)C(O)C(O)C2O)O[C@H]1c1snc2c(N)ncnc12] |
| Example 182C | 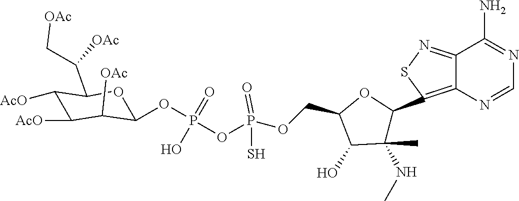 [SMILES: CN[C@]1(C)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1snc2c(N)ncnc12] |
| Example 182D | 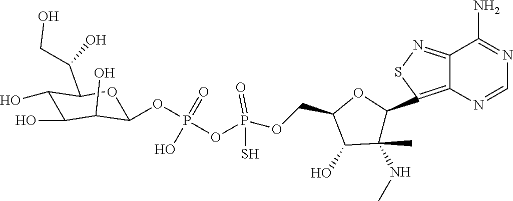 [SMILES: CN[C@]1(C)[C@H](O)[C@@H](COP(=O)(S)OP(=O)(O)OC2OC([C@@H](O)CO)C(O)C(O)C2O)O[C@H]1c1snc2c(N)ncnc12] |
| Example 183A | 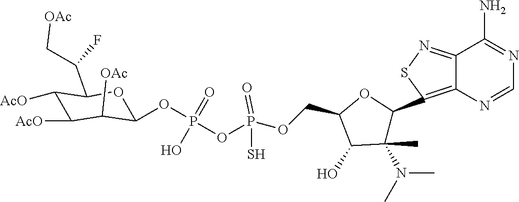 [SMILES: CC(=O)OC[C@H](F)C1OC(OP(=O)(O)OP(=O)(S)OC[C@H]2O[C@@H](c3snc4c(N)ncnc34)[C@](C)(N(C)C)[C@@H]2O)C(OC(C)=O)C(OC(C)=O)C1OC(C)=O] |
| Example 183B | 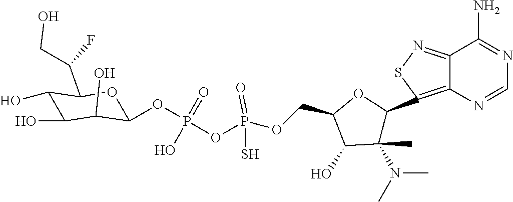 [SMILES: CN(C)[C@]1(C)[C@H](O)[C@@H](COP(=O)(S)OP(=O)(O)OC2OC([C@@H](F)CO)C(O)C(O)C2O)O[C@H]1c1snc2c(N)ncnc12] |
| Example 183C | 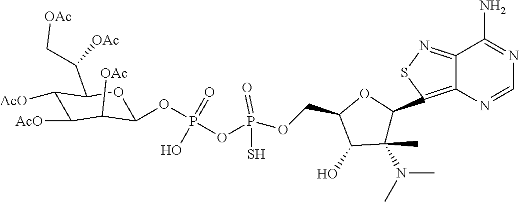 [SMILES: CC(=O)OC[C@H](OC(C)=O)C1OC(OP(=O)(O)OP(=O)(S)OC[C@H]2O[C@@H](c3snc4c(N)ncnc34)[C@](C)(N(C)C)[C@@H]2O)C(OC(C)=O)C(OC(C)=O)C1OC(C)=O] |
| Example 183D | 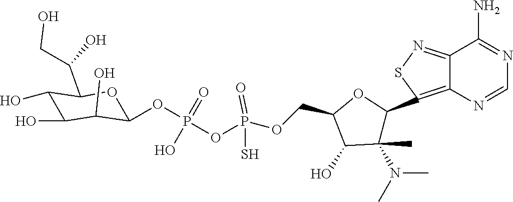 [SMILES: CN(C)[C@]1(C)[C@H](O)[C@@H](COP(=O)(S)OP(=O)(O)OC2OC([C@@H](O)CO)C(O)C(O)C2O)O[C@H]1c1snc2c(N)ncnc12] |
| Example 184A | 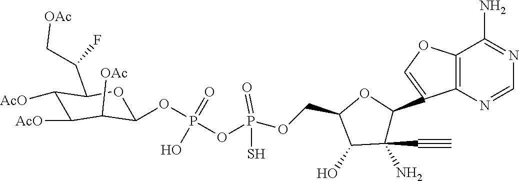 [SMILES: C#C[C@@]1(N)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1coc2c(N)ncnc12] |
| Example 184B | 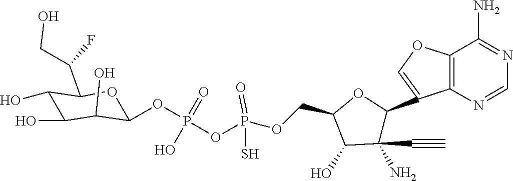 [SMILES: C#C[C@@]1(N)[C@H](O)[C@@H](COP(=O)(S)OP(=O)(O)OC2OC([C@@H](F)CO)C(O)C(O)C2O)O[C@H]1c1coc2c(N)ncnc12] |
| Example 184C | 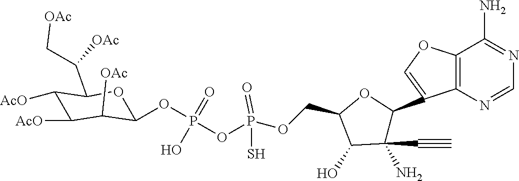 [SMILES: C#C[C@@]1(N)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1coc2c(N)ncnc12] |
| Example 184D | 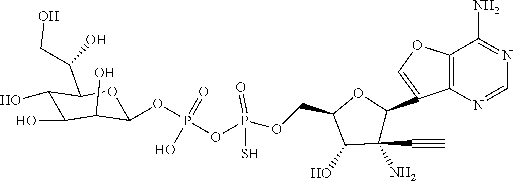 [SMILES: C#C[C@@]1(N)[C@H](O)[C@@H](COP(=O)(S)OP(=O)(O)OC2OC([C@@H](O)CO)C(O)C(O)C2O)O[C@H]1c1coc2c(N)ncnc12] |
| Example 185A | 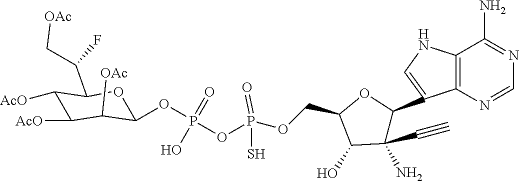 [SMILES: C#C[C@@]1(N)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1c[nH]c2c(N)ncnc12] |
| Example 185B | 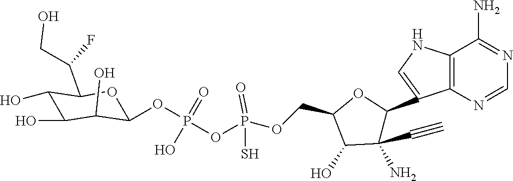 [SMILES: C#C[C@@]1(N)[C@H](O)[C@@H](COP(=O)(S)OP(=O)(O)OC2OC([C@@H](F)CO)C(O)C(O)C2O)O[C@H]1c1c[nH]c2c(N)ncnc12] |
| Example 185C | 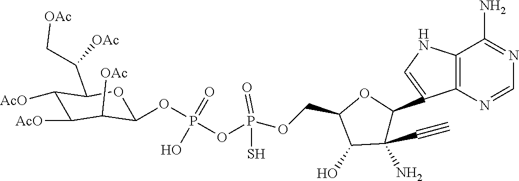 [SMILES: C#C[C@@]1(N)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1c[nH]c2c(N)ncnc12] |
| Example 185D | 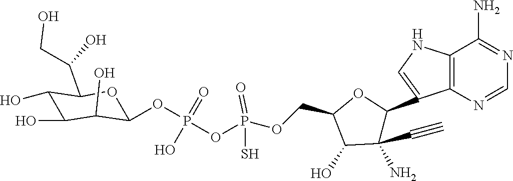 [SMILES: C#C[C@@]1(N)[C@H](O)[C@@H](COP(=O)(S)OP(=O)(O)OC2OC([C@@H](O)CO)C(O)C(O)C2O)O[C@H]1c1c[nH]c2c(N)ncnc12] |
| Example 186A | 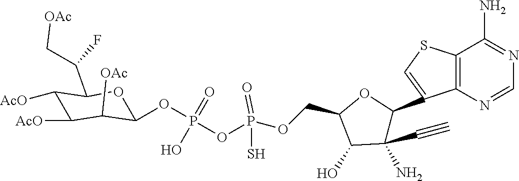 [SMILES: C#C[C@@]1(N)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1csc2c(N)ncnc12] |
| Example 186B | 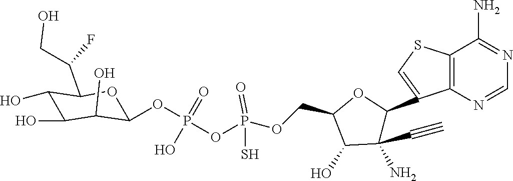 [SMILES: C#C[C@@]1(N)[C@H](O)[C@@H](COP(=O)(S)OP(=O)(O)OC2OC([C@@H](F)CO)C(O)C(O)C2O)O[C@H]1c1csc2c(N)ncnc12] |
| Example 186C | 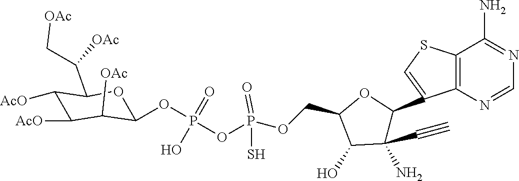 [SMILES: C#C[C@@]1(N)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1csc2c(N)ncnc12] |
| Example 186D | 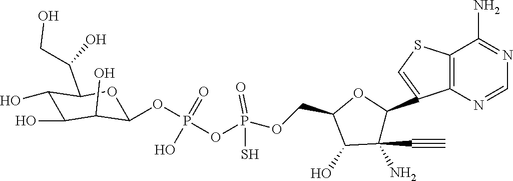 [SMILES: C#C[C@@]1(N)[C@H](O)[C@@H](COP(=O)(S)OP(=O)(O)OC2OC([C@@H](O)CO)C(O)C(O)C2O)O[C@H]1c1csc2c(N)ncnc12] |
| Example 187A | 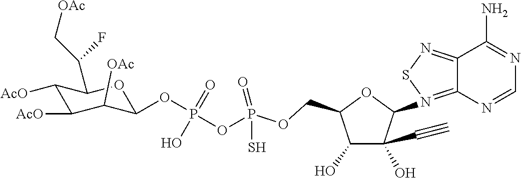 |
| Example 187B | 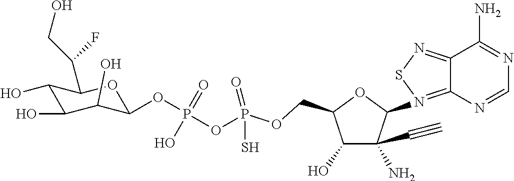 |
| Example 187C | 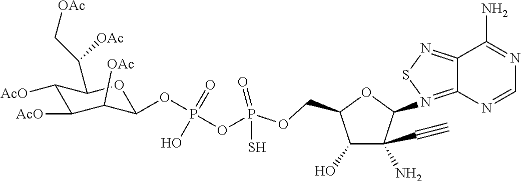 |
| Example 187D | 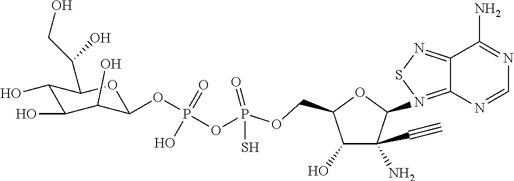 |
| Example 202A | 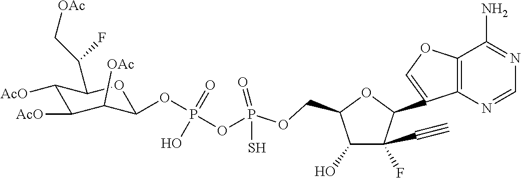 [SMILES: C#C[C@@]1(F)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1coc2c(N)ncnc12] |
| Example 202B | 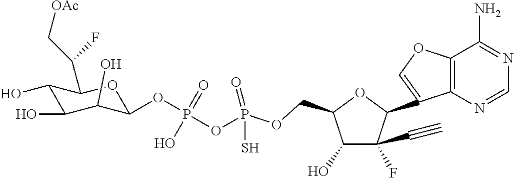 [SMILES: C#C[C@@]1(F)[C@H](O)[C@@H](COP(=O)(S)OP(=O)(O)OC2OC([C@@H](F)COC(C)=O)C(O)C(O)C2O)O[C@H]1c1coc2c(N)ncnc12] |
| Example 202C | 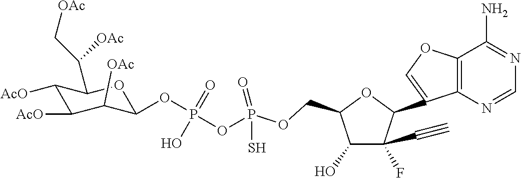 [SMILES: C#C[C@@]1(F)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1coc2c(N)ncnc12] |
| Example 202D | 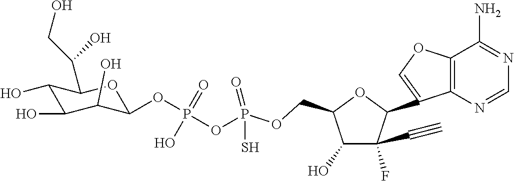 [SMILES: C#C[C@@]1(F)[C@H](O)[C@@H](COP(=O)(S)OP(=O)(O)OC2OC([C@@H](O)CO)C(O)C(O)C2O)O[C@H]1c1coc2c(N)ncnc12] |
| Example 203A | 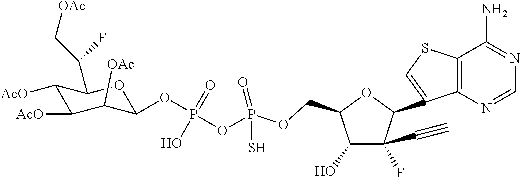 [SMILES: C#C[C@@]1(F)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1csc2c(N)ncnc12] |
| Example 203B | 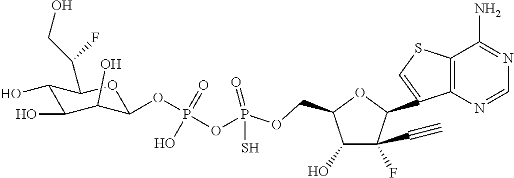 [SMILES: C#C[C@@]1(F)[C@H](O)[C@@H](COP(=O)(S)OP(=O)(O)OC2OC([C@@H](F)CO)C(O)C(O)C2O)O[C@H]1c1csc2c(N)ncnc12] |
| Example 203C | 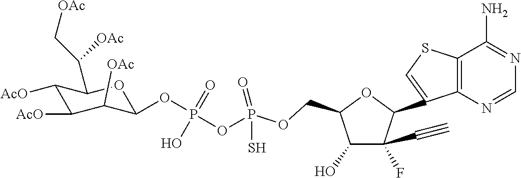 [SMILES: C#C[C@@]1(F)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1csc2c(N)ncnc12] |
| Example 203D | 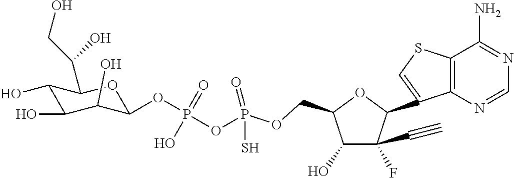 [SMILES: C#C[C@@]1(F)[C@H](O)[C@@H](COP(=O)(S)OP(=O)(O)OC2OC([C@@H](O)CO)C(O)C(O)C2O)O[C@H]1c1csc2c(N)ncnc12] |
| Example 204A | 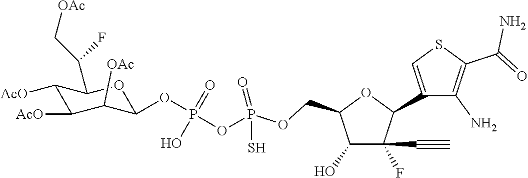 [SMILES: C#C[C@@]1(F)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1csc(C(N)=O)c1N] |
| Example 204B | 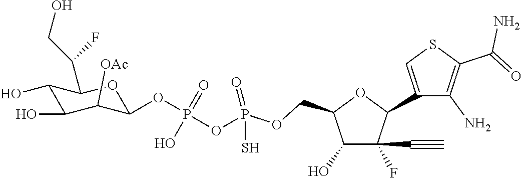 [SMILES: C#C[C@@]1(F)[C@H](O)[C@@H](COP(=O)(S)OP(=O)(O)OC2OC([C@@H](F)CO)C(O)C(O)C2OC(C)=O)O[C@H]1c1csc(C(N)=O)c1N] |
| Example 204C | 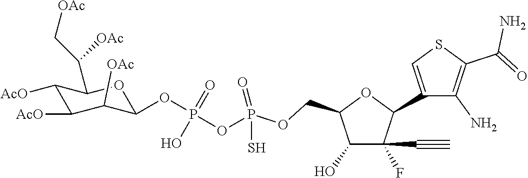 [SMILES: C#C[C@@]1(F)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1csc(C(N)=O)c1N] |
| Example 204D | 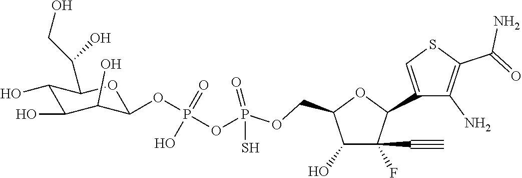 [SMILES: C#C[C@@]1(F)[C@H](O)[C@@H](COP(=O)(S)OP(=O)(O)OC2OC([C@@H](O)CO)C(O)C(O)C2O)O[C@H]1c1csc(C(N)=O)c1N] |
| Example 205A | 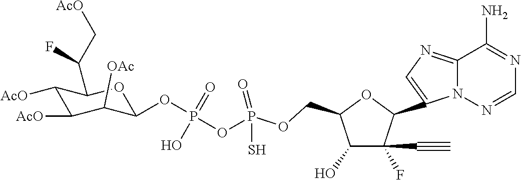 [SMILES: C#C[C@@]1(F)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1cnc2c(N)ncnn12] |
| Example 205B | 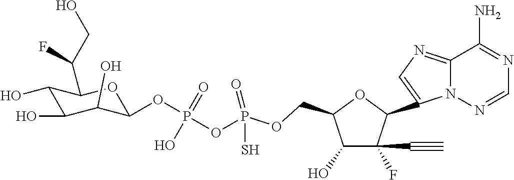 [SMILES: C#C[C@@]1(F)[C@H](O)[C@@H](COP(=O)(S)OP(=O)(O)OC2OC([C@@H](F)CO)C(O)C(O)C2O)O[C@H]1c1cnc2c(N)ncnn12] |
| Example 205C | 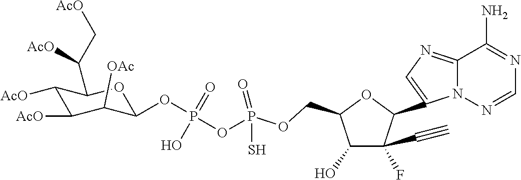 [SMILES: C#C[C@@]1(F)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1cnc2c(N)ncnn12] |
| Example 205D | 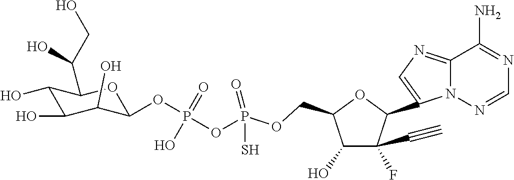 [SMILES: C#C[C@@]1(F)[C@H](O)[C@@H](COP(=O)(S)OP(=O)(O)OC2OC([C@@H](O)CO)C(O)C(O)C2O)O[C@H]1c1cnc2c(N)ncnn12] |
| Example 206A | 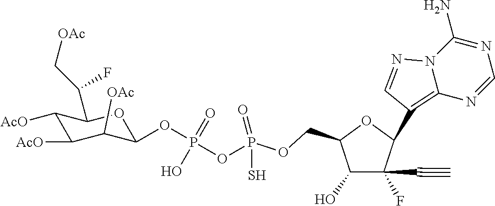 [SMILES: C#C[C@@]1(F)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1cnn2c(N)ncnc12] |
| Example 206B | 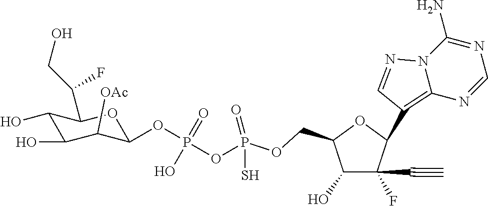 [SMILES: C#C[C@@]1(F)[C@H](O)[C@@H](COP(=O)(S)OP(=O)(O)OC2OC([C@@H](F)CO)C(O)C(O)C2OC(C)=O)O[C@H]1c1cnn2c(N)ncnc12] |
| Example 206C | 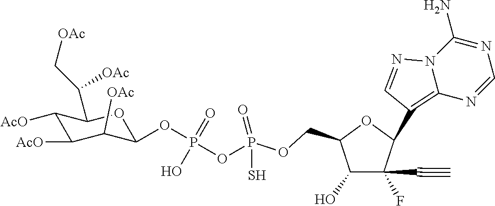 [SMILES: C#C[C@@]1(F)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1cnn2c(N)ncnc12] |
| Example 206D | 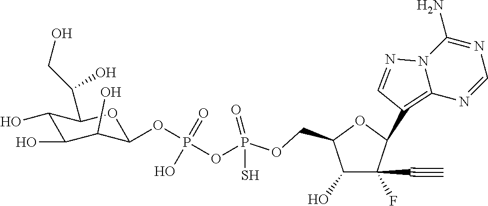 [SMILES: C#C[C@@]1(F)[C@H](O)[C@@H](COP(=O)(S)OP(=O)(O)OC2OC([C@@H](O)CO)C(O)C(O)C2O)O[C@H]1c1cnn2c(N)ncnc12] |
| Example 207A | 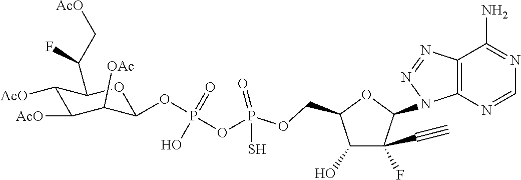 [SMILES: C#C[C@@]1(F)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1nnc2c(N)ncnc21] |
| Example 207B | 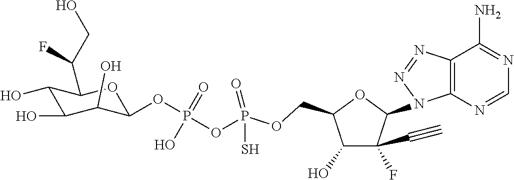 [SMILES: C#C[C@@]1(F)[C@H](O)[C@@H](COP(=O)(S)OP(=O)(O)OC2OC([C@@H](F)CO)C(O)C(O)C2O)O[C@H]1n1nnc2c(N)ncnc21] |
| Example 207C | 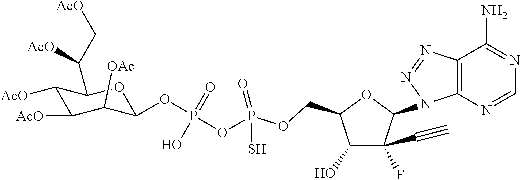 [SMILES: C#C[C@@]1(F)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1nnc2c(N)ncnc21] |
| Example 207D | 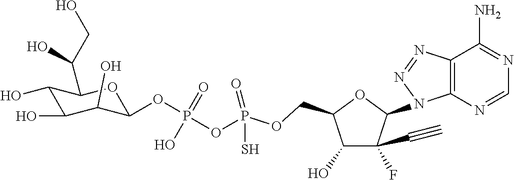 [SMILES: C#C[C@@]1(F)[C@H](O)[C@@H](COP(=O)(S)OP(=O)(O)OC2OC([C@@H](O)CO)C(O)C(O)C2O)O[C@H]1n1nnc2c(N)ncnc21] |
| Example 208A | 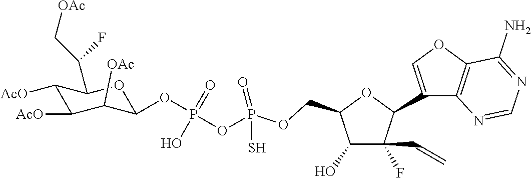 [SMILES: C=C[C@@]1(F)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1coc2c(N)ncnc12] |
| Example 208B | 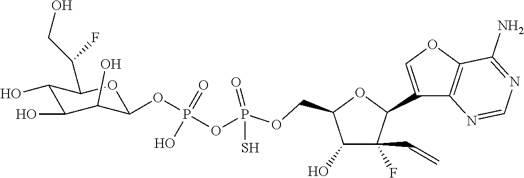 [SMILES: C=C[C@@]1(F)[C@H](O)[C@@H](COP(=O)(S)OP(=O)(O)OC2OC([C@@H](F)CO)C(O)C(O)C2O)O[C@H]1c1coc2c(N)ncnc12] |
| Example 208C | 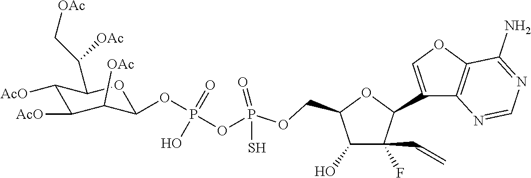 [SMILES: C=C[C@@]1(F)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1coc2c(N)ncnc12] |
| Example 208D | 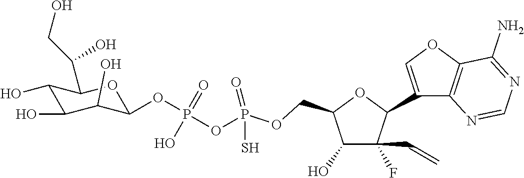 [SMILES: C=C[C@@]1(F)[C@H](O)[C@@H](COP(=O)(S)OP(=O)(O)OC2OC([C@@H](O)CO)C(O)C(O)C2O)O[C@H]1c1coc2c(N)ncnc12] |
| Example 209A | 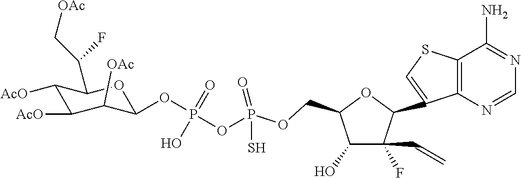 [SMILES: C=C[C@@]1(F)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1csc2c(N)ncnc12] |
| Example 209B | 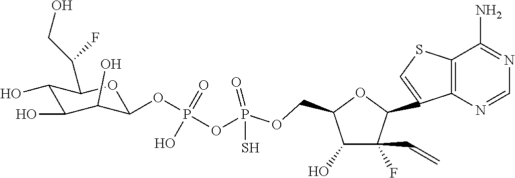 [SMILES: C=C[C@@]1(F)[C@H](O)[C@@H](COP(=O)(S)OP(=O)(O)OC2OC([C@@H](F)CO)C(O)C(O)C2O)O[C@H]1c1csc2c(N)ncnc12] |
| Example 209C | 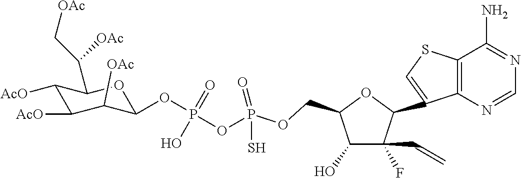 [SMILES: C=C[C@@]1(F)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1csc2c(N)ncnc12] |
| Example 209D | 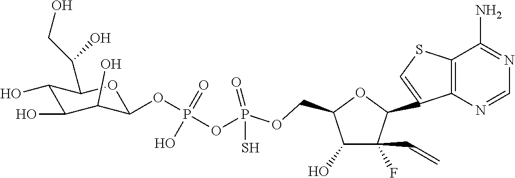 [SMILES: C=C[C@@]1(F)[C@H](O)[C@@H](COP(=O)(S)OP(=O)(O)OC2OC([C@@H](O)CO)C(O)C(O)C2O)O[C@H]1c1csc2c(N)ncnc12] |
| Example 210A | 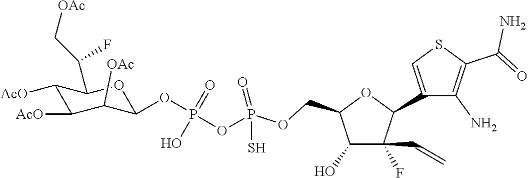 [SMILES: C=C[C@@]1(F)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1csc(C(N)=O)c1N] |
| Example 210B | 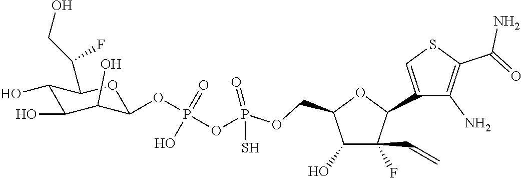 [SMILES: C=C[C@@]1(F)[C@H](O)[C@@H](COP(=O)(S)OP(=O)(O)OC2OC([C@@H](F)CO)C(O)C(O)C2O)O[C@H]1c1csc(C(N)=O)c1N] |
| Example 210C | 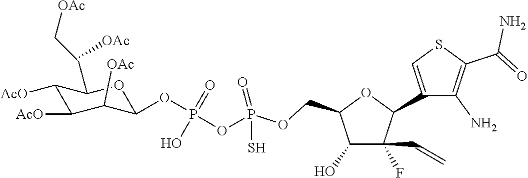 |
| Example 210D | 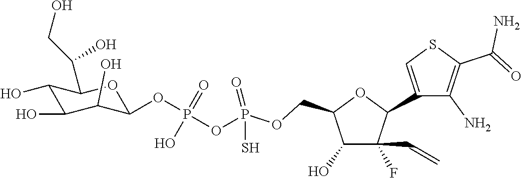 [SMILES: C=C[C@@]1(F)[C@H](O)[C@@H](COP(=O)(S)OP(=O)(O)OC2OC([C@@H](O)CO)C(O)C(O)C2O)O[C@H]1c1csc(C(N)=O)c1N] |
| Example 211A | 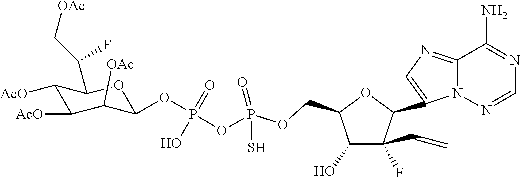 [SMILES: C=C[C@@]1(F)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1cnc2c(N)ncnn12] |
| Example 211B | 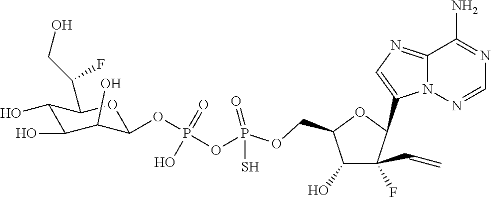 [SMILES: C=C[C@@]1(F)[C@H](O)[C@@H](COP(=O)(S)OP(=O)(O)OC2OC([C@@H](F)CO)C(O)C(O)C2O)O[C@H]1c1cnc2c(N)ncnn12] |
| Example 211C | 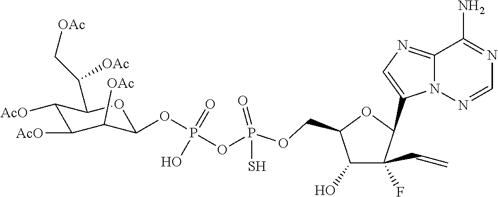 [SMILES: C=C[C@@]1(F)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1cnc2c(N)ncnn12] |
| Example 211D | 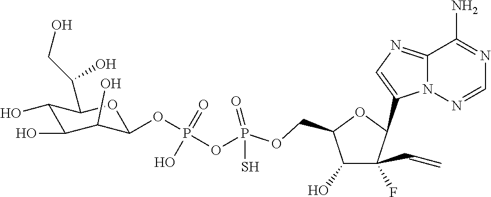 [SMILES: C=C[C@@]1(F)[C@H](O)[C@@H](COP(=O)(S)OP(=O)(O)OC2OC([C@@H](O)CO)C(O)C(O)C2O)O[C@H]1c1cnc2c(N)ncnn12] |
| Example 212A | 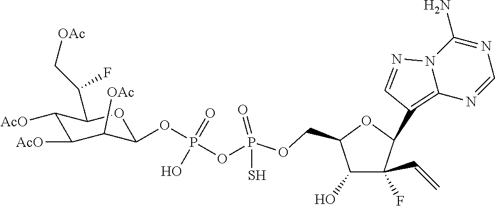 [SMILES: C=C[C@@]1(F)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1cnn2c(N)ncnc12] |
| Example 212B | 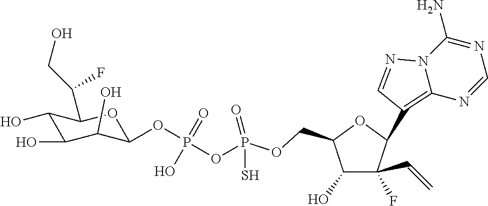 [SMILES: C=C[C@@]1(F)[C@H](O)[C@@H](COP(=O)(S)OP(=O)(O)OC2OC([C@@H](F)CO)C(O)C(O)C2O)O[C@H]1c1cnn2c(N)ncnc12] |
| Example 212C | 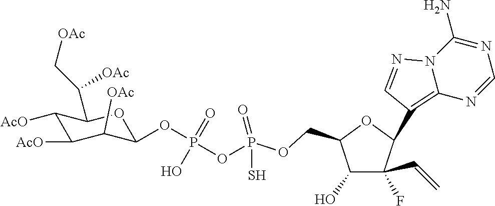 [SMILES: C=C[C@@]1(F)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1cnn2c(N)ncnc12] |
| Example 212D | 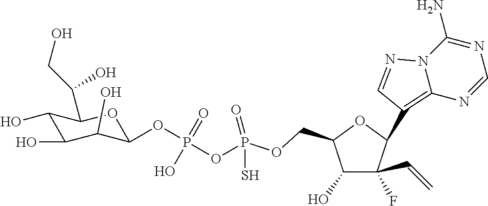 [SMILES: C=C[C@@]1(F)[C@H](O)[C@@H](COP(=O)(S)OP(=O)(O)OC2OC([C@@H](O)CO)C(O)C(O)C2O)O[C@H]1c1cnn2c(N)ncnc12] |
| Example 213A | 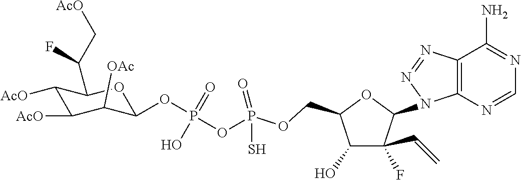 [SMILES: C=C[C@@]1(F)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1nnc2c(N)ncnc21] |
| Example 213B | 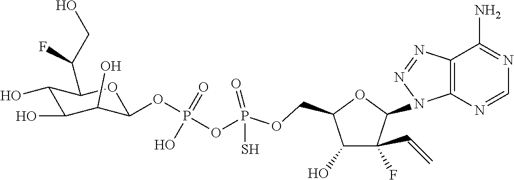 [SMILES: C=C[C@@]1(F)[C@H](O)[C@@H](COP(=O)(S)OP(=O)(O)OC2OC([C@@H](F)CO)C(O)C(O)C2O)O[C@H]1n1nnc2c(N)ncnc21] |
| Example 213C | 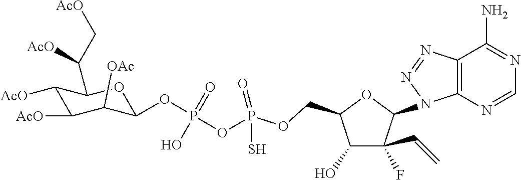 [SMILES: C=C[C@@]1(F)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1nnc2c(N)ncnc21] |
| Example 213D | 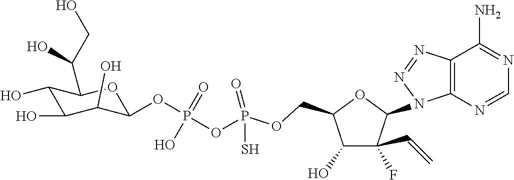 [SMILES: C=C[C@@]1(F)[C@H](O)[C@@H](COP(=O)(S)OP(=O)(O)OC2OC([C@@H](O)CO)C(O)C(O)C2O)O[C@H]1n1nnc2c(N)ncnc21] |
| Example 214A | 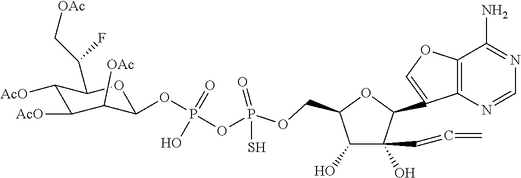 [SMILES: C=C=C[C@@]1(O)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1coc2c(N)ncnc12] |
| Example 214B | 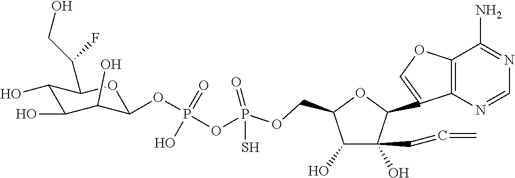 [SMILES: C=C=C[C@@]1(O)[C@H](O)[C@@H](COP(=O)(S)OP(=O)(O)OC2OC([C@@H](F)CO)C(O)C(O)C2O)O[C@H]1c1coc2c(N)ncnc12] |
| Example 214C | 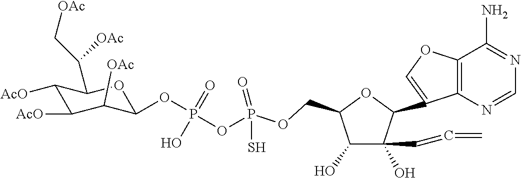 [SMILES: C=C=C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1coc2c(N)ncnc12] |
| Example 214D | 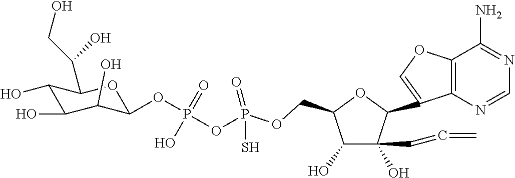 [SMILES: C=C=C[C@@]1(O)[C@H](O)[C@@H](COP(=O)(S)OP(=O)(O)OC2OC([C@@H](O)CO)C(O)C(O)C2O)O[C@H]1c1coc2c(N)ncnc12] |
| Example 215A | 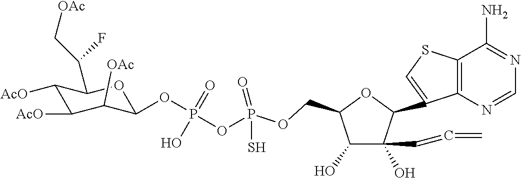 [SMILES: C=C=C[C@@]1(O)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1csc2c(N)ncnc12] |
| Example 215B | 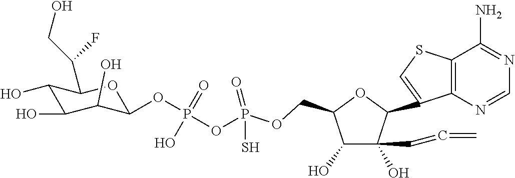 [SMILES: C=C=C[C@@]1(O)[C@H](O)[C@@H](COP(=O)(S)OP(=O)(O)OC2OC([C@@H](F)CO)C(O)C(O)C2O)O[C@H]1c1csc2c(N)ncnc12] |
| Example 215C | 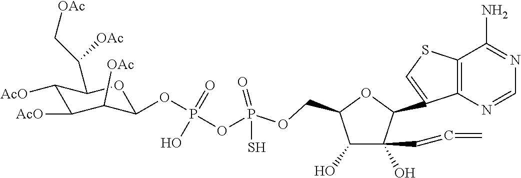 [SMILES: C=C=C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1csc2c(N)ncnc12] |
| Example 215D | 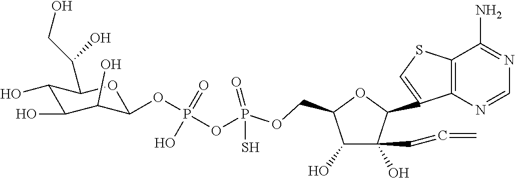 [SMILES: C=C=C[C@@]1(O)[C@H](O)[C@@H](COP(=O)(S)OP(=O)(O)OC2OC([C@@H](O)CO)C(O)C(O)C2O)O[C@H]1c1csc2c(N)ncnc12] |
| Example 216A | 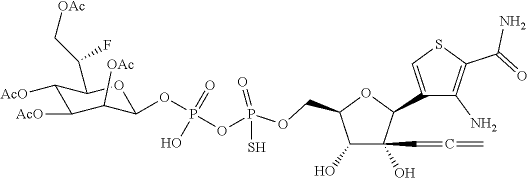 [SMILES: C=C=C[C@@]1(O)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1csc(C(N)=O)c1N] |
| Example 216B | 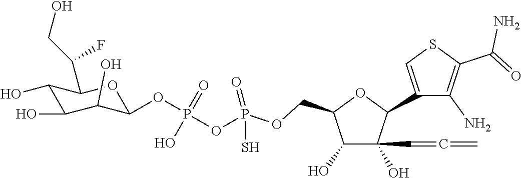 [SMILES: C=C=C[C@@]1(O)[C@H](O)[C@@H](COP(=O)(S)OP(=O)(O)OC2OC([C@@H](F)CO)C(O)C(O)C2O)O[C@H]1c1csc(C(N)=O)c1N] |
| Example 216C | 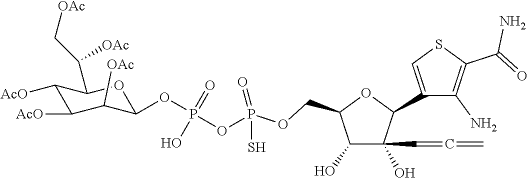 [SMILES: C=C=C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1csc(C(N)=O)c1N] |
| Example 216D | 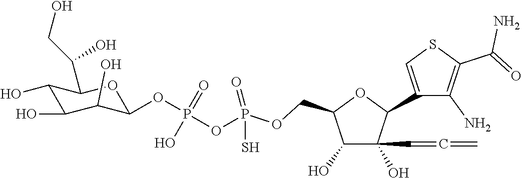 [SMILES: C=C=C[C@@]1(O)[C@H](O)[C@@H](COP(=O)(S)OP(=O)(O)OC2OC([C@@H](O)CO)C(O)C(O)C2O)O[C@H]1c1csc(C(N)=O)c1N] |
| Example 217A | 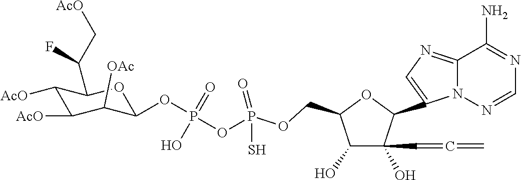 [SMILES: C=C=C[C@@]1(O)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1cnc2c(N)ncnn12] |
| Example 217B | 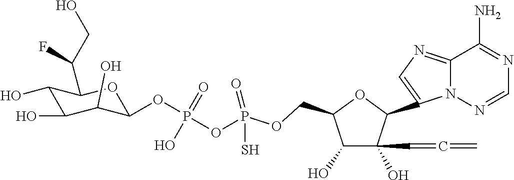 [SMILES: C=C=C[C@@]1(O)[C@H](O)[C@@H](COP(=O)(S)OP(=O)(O)OC2OC([C@@H](F)CO)C(O)C(O)C2O)O[C@H]1c1cnc2c(N)ncnn12] |
| Example 217C | 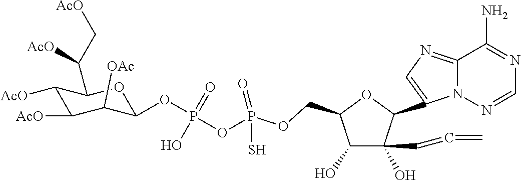 [SMILES: C=C=C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1cnc2c(N)ncnn12] |
| Example 217D | 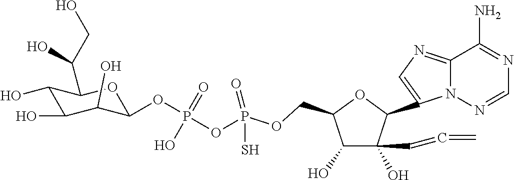 [SMILES: C=C=C[C@@]1(O)[C@H](O)[C@@H](COP(=O)(S)OP(=O)(O)OC2OC([C@@H](O)CO)C(O)C(O)C2O)O[C@H]1c1cnc2c(N)ncnn12] |
| Example 218A | 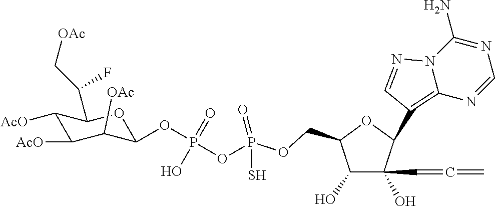 [SMILES: C=C=C[C@@]1(O)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1cnn2c(N)ncnc12] |
| Example 218B | 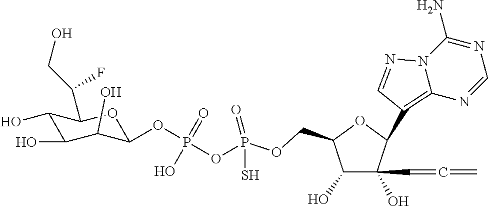 [SMILES: C=C=C[C@@]1(O)[C@H](O)[C@@H](COP(=O)(S)OP(=O)(O)OC2OC([C@@H](F)CO)C(O)C(O)C2O)O[C@H]1c1cnn2c(N)ncnc12] |
| Example 218C | 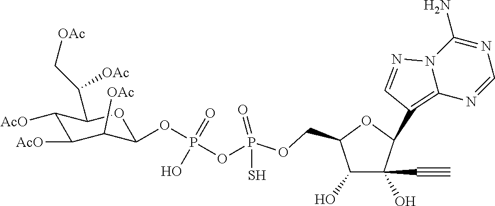 [SMILES: C#C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1cnn2c(N)ncnc12] |
| Example 218D | 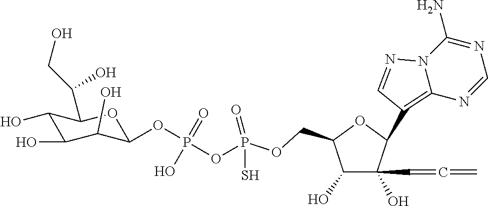 [SMILES: C=C=C[C@@]1(O)[C@H](O)[C@@H](COP(=O)(S)OP(=O)(O)OC2OC([C@@H](O)CO)C(O)C(O)C2O)O[C@H]1c1cnn2c(N)ncnc12] |
| Example 219A | 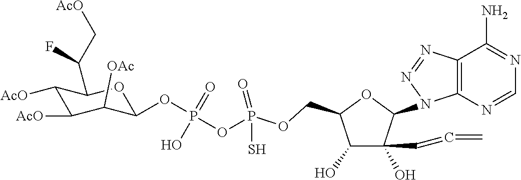 [SMILES: C=C=C[C@@]1(O)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1nnc2c(N)ncnc21] |
| Example 219B | 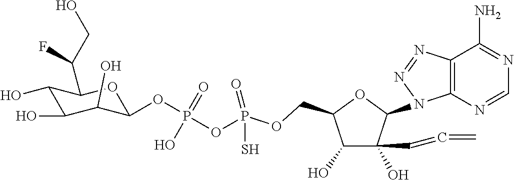 [SMILES: C=C=C[C@@]1(O)[C@H](O)[C@@H](COP(=O)(S)OP(=O)(O)OC2OC([C@@H](F)CO)C(O)C(O)C2O)O[C@H]1n1nnc2c(N)ncnc21] |
| Example 219C | 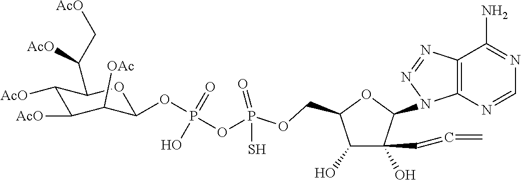 [SMILES: C=C=C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1nnc2c(N)ncnc21] |
| Example 219D | 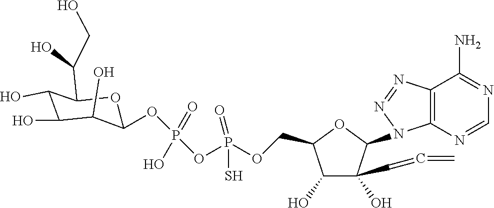 [SMILES: C=C=C[C@@]1(O)[C@H](O)[C@@H](COP(=O)(S)OP(=O)(O)OC2OC([C@@H](O)CO)C(O)C(O)C2O)O[C@H]1n1nnc2c(N)ncnc21] |
| Example 220A | 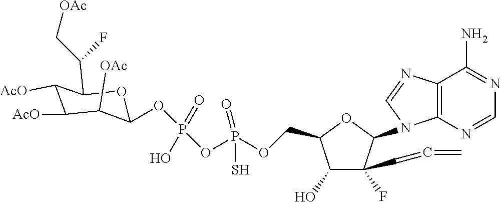 [SMILES: C=C=C[C@@]1(F)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1cnc2c(N)ncnc21] |
| Example 220B | 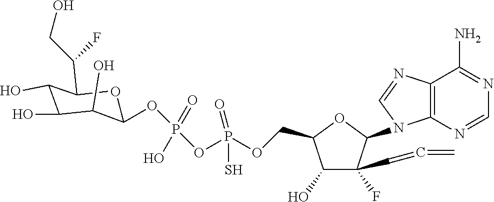 [SMILES: C=C=C[C@@]1(F)[C@H](O)[C@@H](COP(=O)(S)OP(=O)(O)OC2OC([C@@H](F)CO)C(O)C(O)C2O)O[C@H]1n1cnc2c(N)ncnc21] |
| Example 220C | 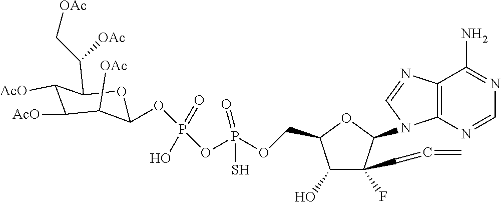 [SMILES: C=C=C[C@@]1(F)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1cnc2c(N)ncnc21] |
| Example 220D | 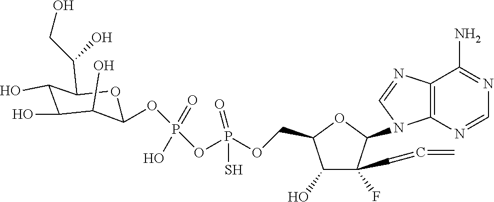 [SMILES: C=C=C[C@@]1(F)[C@H](O)[C@@H](COP(=O)(S)OP(=O)(O)OC2OC([C@@H](O)CO)C(O)C(O)C2O)O[C@H]1n1cnc2c(N)ncnc21] |
| Example 221A | 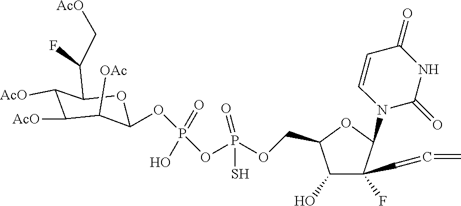 [SMILES: C=C=C[C@@]1(F)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1ccc(=O)[nH]c1=O] |
| Example 221B | 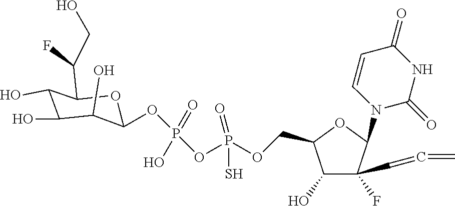 [SMILES: C=C=C[C@@]1(F)[C@H](O)[C@@H](COP(=O)(S)OP(=O)(O)OC2OC([C@@H](F)CO)C(O)C(O)C2O)O[C@H]1n1ccc(=O)[nH]c1=O] |
| Example 221C | 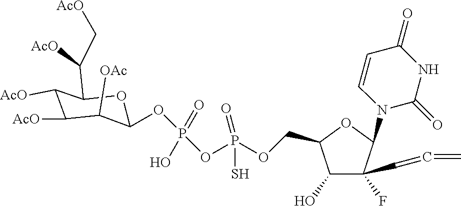 [SMILES: C=C=C[C@@]1(F)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1ccc(=O)[nH]c1=O] |
| Example 221D | 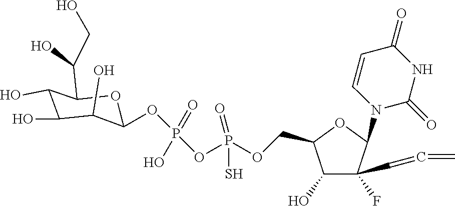 [SMILES: C=C=C[C@@]1(F)[C@H](O)[C@@H](COP(=O)(S)OP(=O)(O)OC2OC([C@@H](O)CO)C(O)C(O)C2O)O[C@H]1n1ccc(=O)[nH]c1=O] |
| Example 222A | 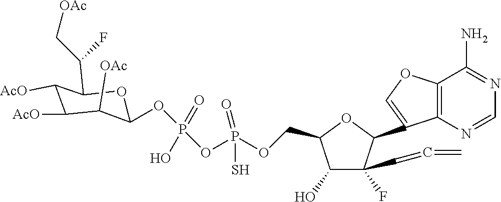 [SMILES: C=C=C[C@@]1(F)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1coc2c(N)ncnc12] |
| Example 222B | 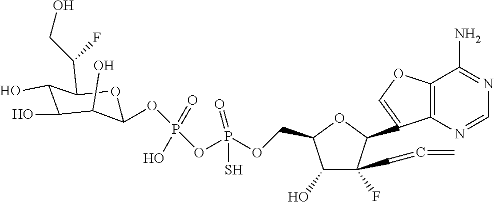 [SMILES: C=C=C[C@@]1(F)[C@H](O)[C@@H](COP(=O)(S)OP(=O)(O)OC2OC([C@@H](F)CO)C(O)C(O)C2O)O[C@H]1c1coc2c(N)ncnc12] |
| Example 222C | 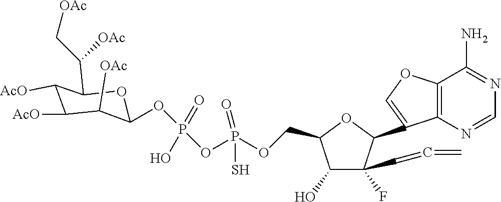 [SMILES: C=C=C[C@@]1(F)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1coc2c(N)ncnc12] |
| Example 222D | 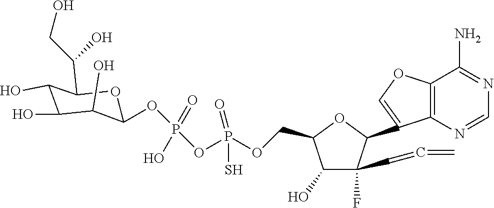 [SMILES: C=C=C[C@@]1(F)[C@H](O)[C@@H](COP(=O)(S)OP(=O)(O)OC2OC([C@@H](O)CO)C(O)C(O)C2O)O[C@H]1c1coc2c(N)ncnc12] |
| Example 223A | 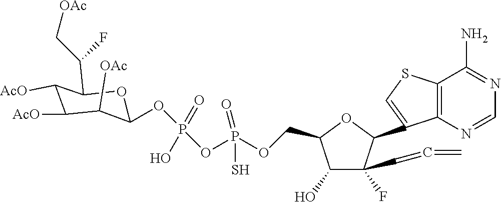 [SMILES: C=C=C[C@@]1(F)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1csc2c(N)ncnc12] |
| Example 223B | 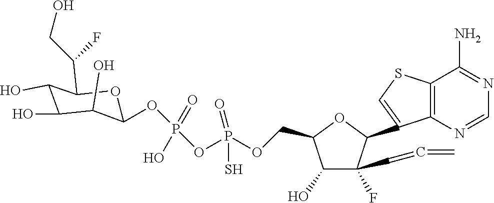 [SMILES: C=C=C[C@@]1(F)[C@H](O)[C@@H](COP(=O)(S)OP(=O)(O)OC2OC([C@@H](F)CO)C(O)C(O)C2O)O[C@H]1c1csc2c(N)ncnc12] |
| Example 223C | 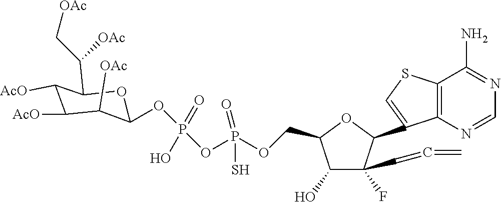 [SMILES: C=C=C[C@@]1(F)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1csc2c(N)ncnc12] |
| Example 223D | 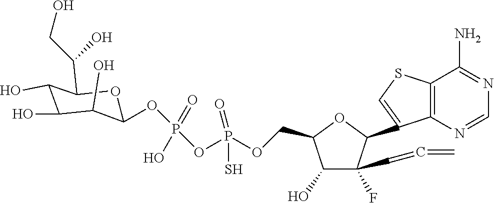 [SMILES: C=C=C[C@@]1(F)[C@H](O)[C@@H](COP(=O)(S)OP(=O)(O)OC2OC([C@@H](O)CO)C(O)C(O)C2O)O[C@H]1c1csc2c(N)ncnc12] |
| Example 224A | 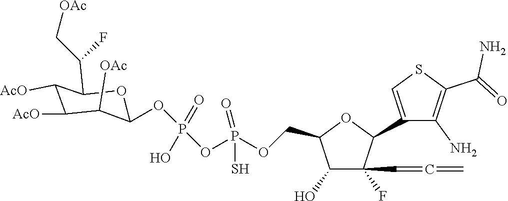 [SMILES: C=C=C[C@@]1(F)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1csc(C(N)=O)c1N] |
| Example 224B | 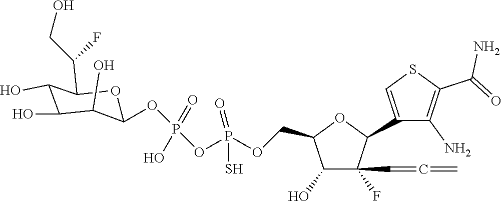 [SMILES: C=C=C[C@@]1(F)[C@H](O)[C@@H](COP(=O)(S)OP(=O)(O)OC2OC([C@@H](F)CO)C(O)C(O)C2O)O[C@H]1c1csc(C(N)=O)c1N] |
| Example 224C | 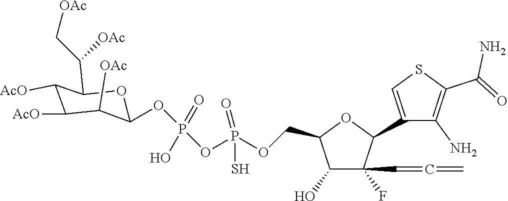 [SMILES: C=C=C[C@@]1(F)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1csc(C(N)=O)c1N] |
| Example 224D | 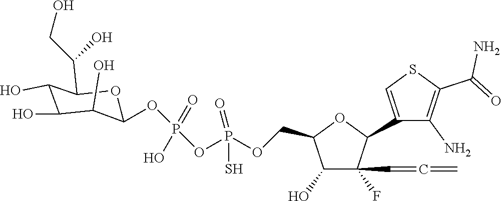 [SMILES: C=C=C[C@@]1(F)[C@H](O)[C@@H](COP(=O)(S)OP(=O)(O)OC2OC([C@@H](O)CO)C(O)C(O)C2O)O[C@H]1c1csc(C(N)=O)c1N] |
| Example 225A | 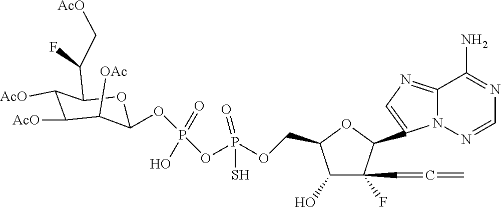 [SMILES: C=C=C[C@@]1(F)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1cnc2c(N)ncnn12] |
| Example 225B | 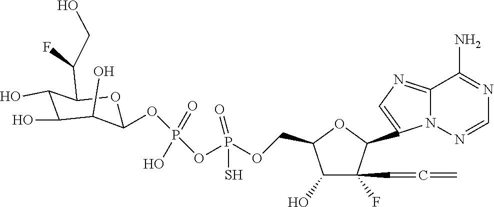 [SMILES: C=C=C[C@@]1(F)[C@H](O)[C@@H](COP(=O)(S)OP(=O)(O)OC2OC([C@@H](F)CO)C(O)C(O)C2O)O[C@H]1c1cnc2c(N)ncnn12] |
| Example 225C | 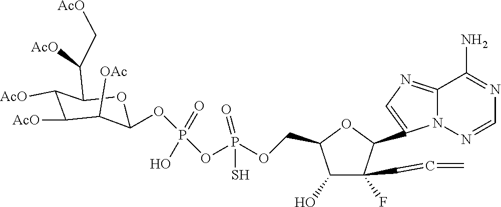 [SMILES: C=C=C[C@@]1(F)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1cnc2c(N)ncnn12] |
| Example 225D | 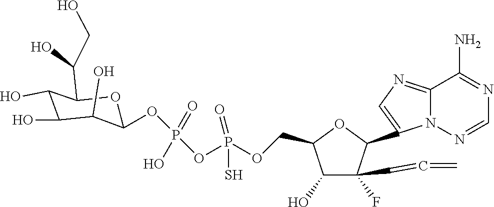 [SMILES: C=C=C[C@@]1(F)[C@H](O)[C@@H](COP(=O)(S)OP(=O)(O)OC2OC([C@@H](O)CO)C(O)C(O)C2O)O[C@H]1c1cnc2c(N)ncnn12] |
| Example 226A | 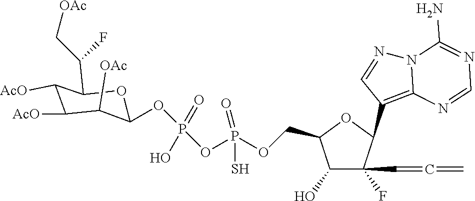 [SMILES: C=C=C[C@@]1(F)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1cnn2c(N)ncnc12] |
| Example 226B | 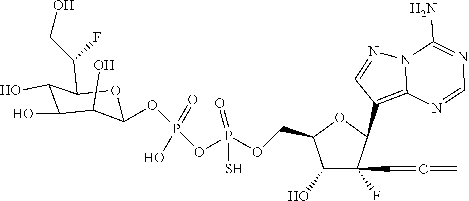 [SMILES: C=C=C[C@@]1(F)[C@H](O)[C@@H](COP(=O)(S)OP(=O)(O)OC2OC([C@@H](F)CO)C(O)C(O)C2O)O[C@H]1c1cnn2c(N)ncnc12] |
| Example 226C | 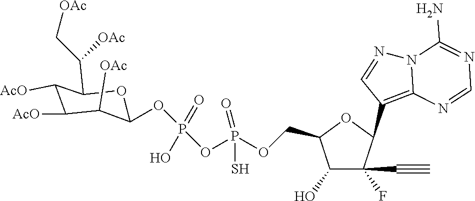 [SMILES: C#C[C@@]1(F)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1cnn2c(N)ncnc12] |
| Example 226D | 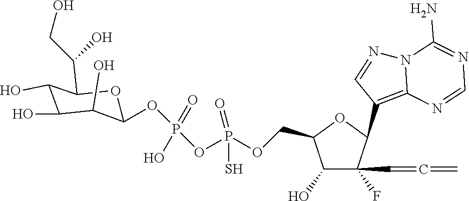 [SMILES: C=C=C[C@@]1(F)[C@H](O)[C@@H](COP(=O)(S)OP(=O)(O)OC2OC([C@@H](O)CO)C(O)C(O)C2O)O[C@H]1c1cnn2c(N)ncnc12] |
| Example 227A | 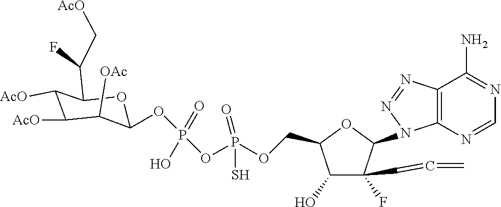 [SMILES: C=C=C[C@@]1(F)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1n1nnc2c(N)ncnc21] |
| Example 227B | 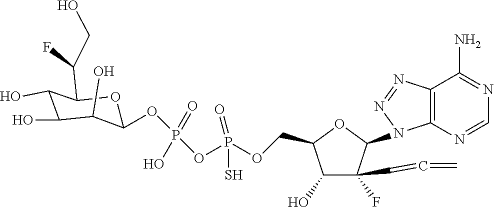 [SMILES: C=C=C[C@@]1(F)[C@H](O)[C@@H](COP(=O)(S)OP(=O)(O)OC2OC([C@@H](F)CO)C(O)C(O)C2O)O[C@H]1n1nnc2c(N)ncnc21] |
| Example 227C | 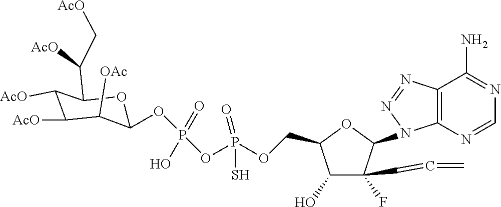 [SMILES: C=C=C[C@@]1(F)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1nnc2c(N)ncnc21] |
| Example 227D | 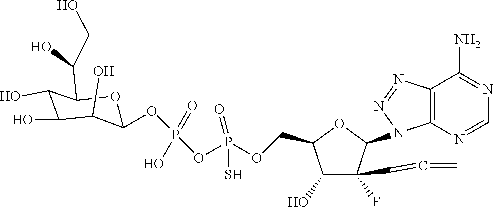 [SMILES: C=C=C[C@@]1(F)[C@H](O)[C@@H](COP(=O)(S)OP(=O)(O)OC2OC([C@@H](O)CO)C(O)C(O)C2O)O[C@H]1n1nnc2c(N)ncnc21] |
| Example 228A | 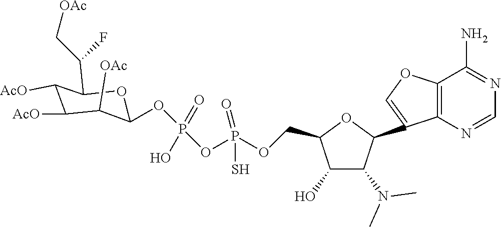 [SMILES: CC(=O)OC[C@H](F)C1OC(OP(=O)(O)OP(=O)(S)OC[C@H]2O[C@@H](c3coc4c(N)ncnc34)[C@H](N(C)C)[C@@H]2O)C(OC(C)=O)C(OC(C)=O)C1OC(C)=O] |
| Example 228B | 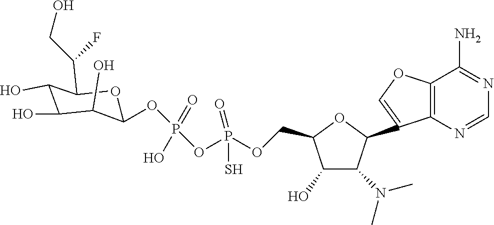 [SMILES: CN(C)[C@@H]1[C@H](O)[C@@H](COP(=O)(S)OP(=O)(O)OC2OC([C@@H](F)CO)C(O)C(O)C2O)O[C@H]1c1coc2c(N)ncnc12] |
| Example 228C | 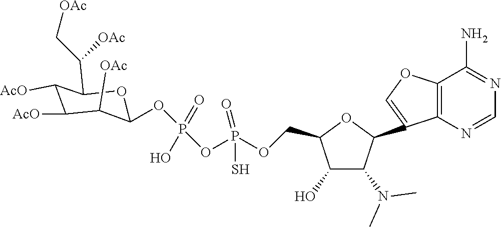 [SMILES: CC(=O)OC[C@H](OC(C)=O)C1OC(OP(=O)(O)OP(=O)(S)OC[C@H]2O[C@@H](c3coc4c(N)ncnc34)[C@H](N(C)C)[C@@H]2O)C(OC(C)=O)C(OC(C)=O)C1OC(C)=O] |
| Example 228D | 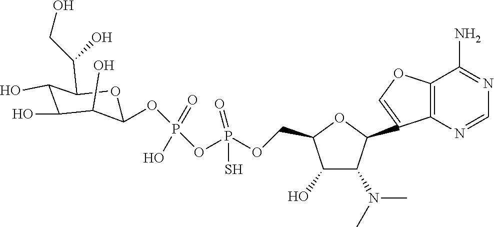 [SMILES: CN(C)[C@@H]1[C@H](O)[C@@H](COP(=O)(S)OP(=O)(O)OC2OC([C@@H](O)CO)C(O)C(O)C2O)O[C@H]1c1coc2c(N)ncnc12] |
| Example 229A | 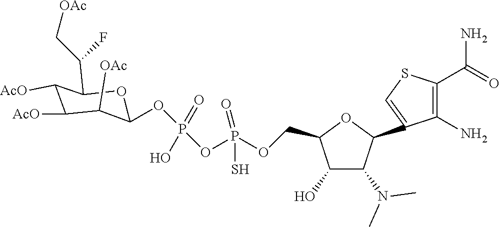 [SMILES: CC(=O)OC[C@H](F)C1OC(OP(=O)(O)OP(=O)(S)OC[C@H]2O[C@@H](c3csc(C(N)=O)c3N)[C@H](N(C)C)[C@@H]2O)C(OC(C)=O)C(OC(C)=O)C1OC(C)=O] |
| Example 229B | 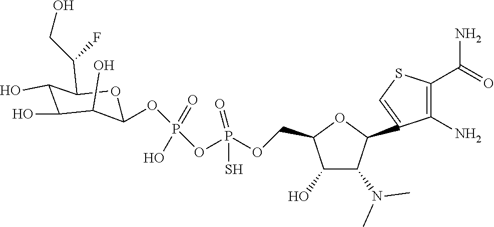 [SMILES: CN(C)[C@@H]1[C@H](O)[C@@H](COP(=O)(S)OP(=O)(O)OC2OC([C@@H](F)CO)C(O)C(O)C2O)O[C@H]1c1csc(C(N)=O)c1N] |
| Example 229C | 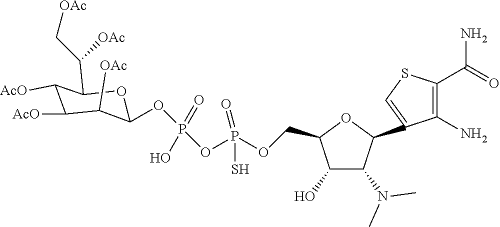 [SMILES: CC(=O)OC[C@H](OC(C)=O)C1OC(OP(=O)(O)OP(=O)(S)OC[C@H]2O[C@@H](c3csc(C(N)=O)c3N)[C@H](N(C)C)[C@@H]2O)C(OC(C)=O)C(OC(C)=O)C1OC(C)=O] |
| Example 229D | 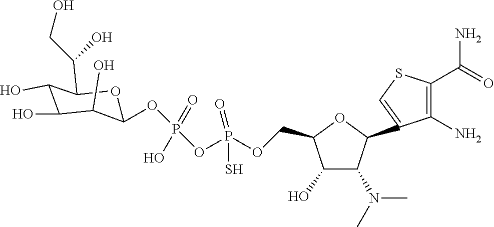 [SMILES: CN(C)[C@@H]1[C@H](O)[C@@H](COP(=O)(S)OP(=O)(O)OC2OC([C@@H](O)CO)C(O)C(O)C2O)O[C@H]1c1csc(C(N)=O)c1N] |
| Example 230A | 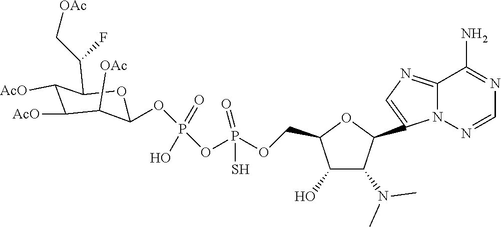 [SMILES: CC(=O)OC[C@H](F)C1OC(OP(=O)(O)OP(=O)(S)OC[C@H]2O[C@@H](c3cnc4c(N)ncnn34)[C@H](N(C)C)[C@@H]2O)C(OC(C)=O)C(OC(C)=O)C1OC(C)=O] |
| Example 230B | 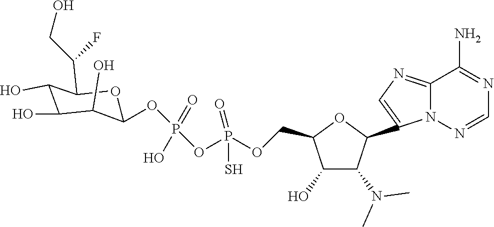 [SMILES: CN(C)[C@@H]1[C@H](O)[C@@H](COP(=O)(S)OP(=O)(O)OC2OC([C@@H](F)CO)C(O)C(O)C2O)O[C@H]1c1cnc2c(N)ncnn12] |
| Example 230C | 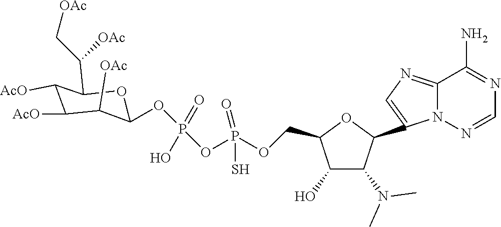 [SMILES: CC(=O)OC[C@H](OC(C)=O)C1OC(OP(=O)(O)OP(=O)(S)OC[C@H]2O[C@@H](c3cnc4c(N)ncnn34)[C@H](N(C)C)[C@@H]2O)C(OC(C)=O)C(OC(C)=O)C1OC(C)=O] |
| Example 230D | 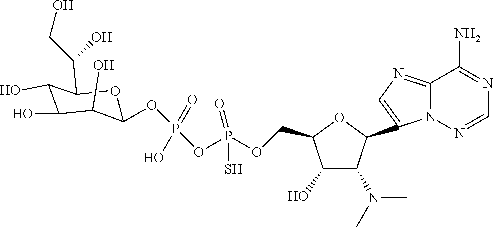 [SMILES: CN(C)[C@@H]1[C@H](O)[C@@H](COP(=O)(S)OP(=O)(O)OC2OC([C@@H](O)CO)C(O)C(O)C2O)O[C@H]1c1cnc2c(N)ncnn12] |
| Example 231A | 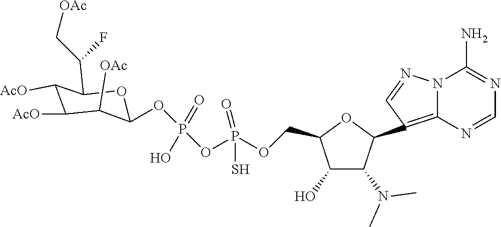 [SMILES: CC(=O)OC[C@H](F)C1OC(OP(=O)(O)OP(=O)(S)OC[C@H]2O[C@@H](c3cnn4c(N)ncnc34)[C@H](N(C)C)[C@@H]2O)C(OC(C)=O)C(OC(C)=O)C1OC(C)=O] |
| Example 231B | 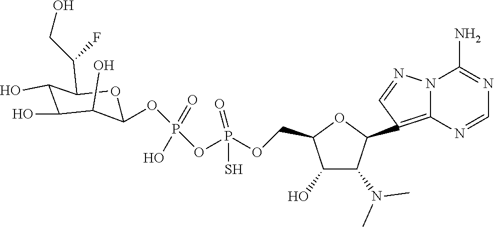 [SMILES: CN(C)[C@@H]1[C@H](O)[C@@H](COP(=O)(S)OP(=O)(O)OC2OC([C@@H](F)CO)C(O)C(O)C2O)O[C@H]1c1cnn2c(N)ncnc12] |
| Example 231C | 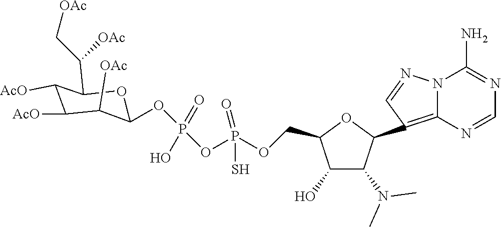 [SMILES: CC(=O)OC[C@H](OC(C)=O)C1OC(OP(=O)(O)OP(=O)(S)OC[C@H]2O[C@@H](c3cnn4c(N)ncnc34)[C@H](N(C)C)[C@@H]2O)C(OC(C)=O)C(OC(C)=O)C1OC(C)=O] |
| Example 231D | 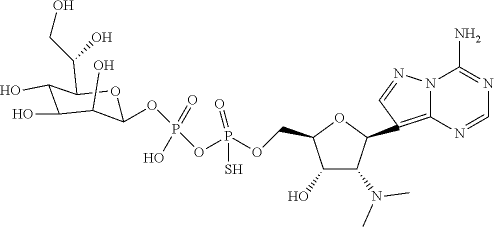 [SMILES: CN(C)[C@@H]1[C@H](O)[C@@H](COP(=O)(S)OP(=O)(O)OC2OC([C@@H](O)CO)C(O)C(O)C2O)O[C@H]1c1cnn2c(N)ncnc12] |
| Example 232A | 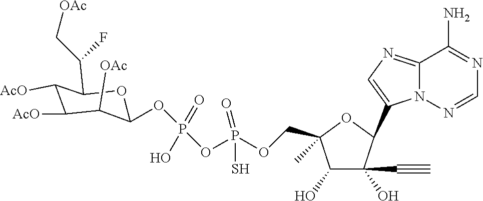 [SMILES: C#C[C@]1(O)[C@H](c2cnc3c(N)ncnn23)O[C@](C)(COP(=O)(S)OP(=O)(O)OC2OC([C@@H](F)COC(C)=O)C(OC(C)=O)C(OC(C)=O)C2OC(C)=O)[C@H]1O] |
| Example 232B | 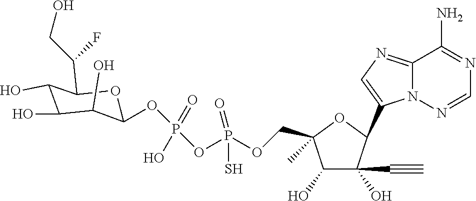 [SMILES: C#C[C@]1(O)[C@H](c2cnc3c(N)ncnn23)O[C@](C)(COP(=O)(S)OP(=O)(O)OC2OC([C@@H](F)CO)C(O)C(O)C2O)[C@H]1O] |
| Example 232C | 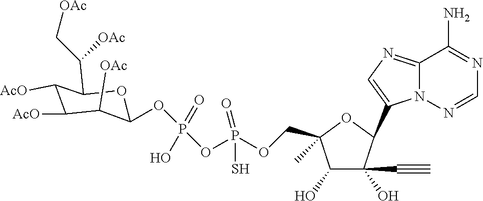 [SMILES: C#C[C@]1(O)[C@H](c2cnc3c(N)ncnn23)O[C@](C)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 232D | 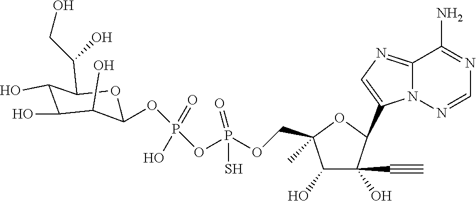 [SMILES: C#C[C@]1(O)[C@H](c2cnc3c(N)ncnn23)O[C@](C)(COP(=O)(S)OP(=O)(O)OC2OC([C@@H](O)CO)C(O)C(O)C2O)[C@H]1O] |
| Example 233A | 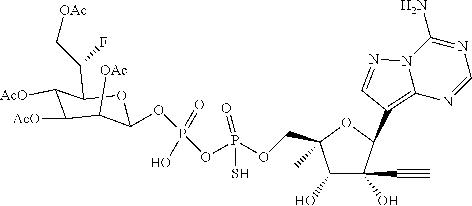 [SMILES: C#C[C@]1(O)[C@H](c2cnn3c(N)ncnc23)O[C@](C)(COP(=O)(S)OP(=O)(O)OC2OC([C@@H](F)COC(C)=O)C(OC(C)=O)C(OC(C)=O)C2OC(C)=O)[C@H]1O] |
| Example 233B | 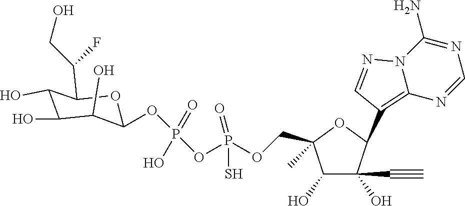 [SMILES: C#C[C@]1(O)[C@H](c2cnn3c(N)ncnc23)O[C@](C)(COP(=O)(S)OP(=O)(O)OC2OC([C@@H](F)CO)C(O)C(O)C2O)[C@H]1O] |
| Example 233C | 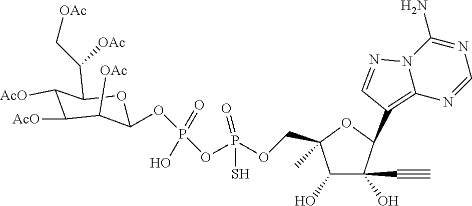 [SMILES: C#C[C@]1(O)[C@H](c2cnn3c(N)ncnc23)O[C@](C)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 233D | 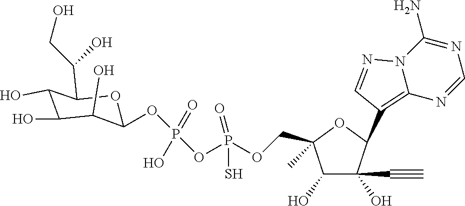 [SMILES: C#C[C@]1(O)[C@H](c2cnn3c(N)ncnc23)O[C@](C)(COP(=O)(S)OP(=O)(O)OC2OC([C@@H](O)CO)C(O)C(O)C2O)[C@H]1O] |
| Example 234A | 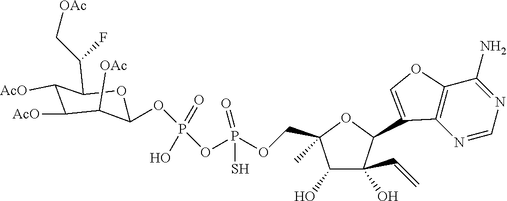 [SMILES: C=C[C@]1(O)[C@H](c2coc3c(N)ncnc23)O[C@](C)(COP(=O)(S)OP(=O)(O)OC2OC([C@@H](F)COC(C)=O)C(OC(C)=O)C(OC(C)=O)C2OC(C)=O)[C@H]1O] |
| Example 234B | 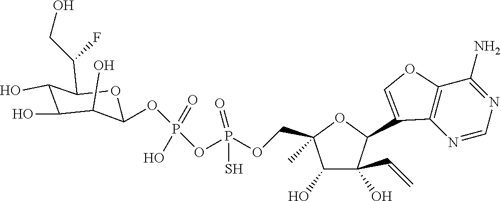 [SMILES: C=C[C@]1(O)[C@H](c2coc3c(N)ncnc23)O[C@](C)(COP(=O)(S)OP(=O)(O)OC2OC([C@@H](F)CO)C(O)C(O)C2O)[C@H]1O] |
| Example 234C | 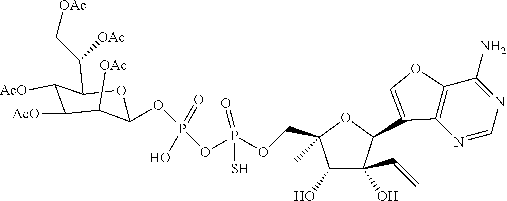 [SMILES: C=C[C@]1(O)[C@H](c2coc3c(N)ncnc23)O[C@](C)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 234D | 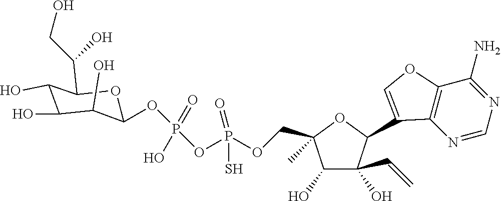 [SMILES: C=C[C@]1(O)[C@H](c2coc3c(N)ncnc23)O[C@](C)(COP(=O)(S)OP(=O)(O)OC2OC([C@@H](O)CO)C(O)C(O)C2O)[C@H]1O] |
| Example 235A | 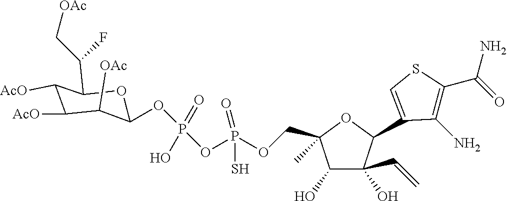 [SMILES: C=C[C@]1(O)[C@H](c2csc(C(N)=O)c2N)O[C@](C)(COP(=O)(S)OP(=O)(O)OC2OC([C@@H](F)COC(C)=O)C(OC(C)=O)C(OC(C)=O)C2OC(C)=O)[C@H]1O] |
| Example 235B | 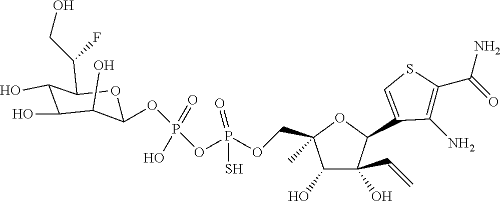 [SMILES: C=C[C@]1(O)[C@H](c2csc(C(N)=O)c2N)O[C@](C)(COP(=O)(S)OP(=O)(O)OC2OC([C@@H](F)CO)C(O)C(O)C2O)[C@H]1O] |
| Example 235C | 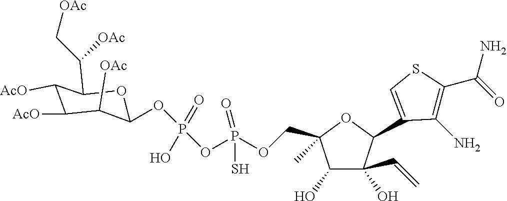 [SMILES: C=C[C@]1(O)[C@H](c2csc(C(N)=O)c2N)O[C@](C)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 235D | 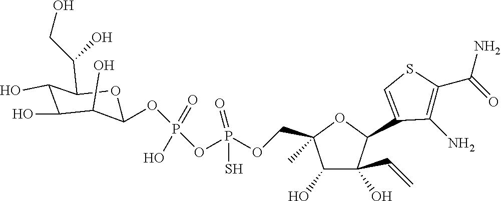 [SMILES: C=C[C@]1(O)[C@H](c2csc(C(N)=O)c2N)O[C@](C)(COP(=O)(S)OP(=O)(O)OC2OC([C@@H](O)CO)C(O)C(O)C2O)[C@H]1O] |
| Example 236A | 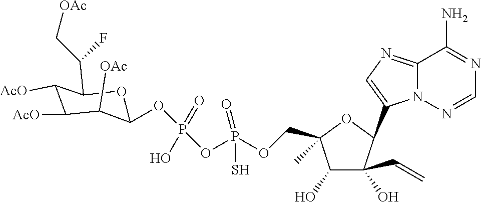 [SMILES: C=C[C@]1(O)[C@H](c2cnc3c(N)ncnn23)O[C@](C)(COP(=O)(S)OP(=O)(O)OC2OC([C@@H](F)COC(C)=O)C(OC(C)=O)C(OC(C)=O)C2OC(C)=O)[C@H]1O] |
| Example 236B | 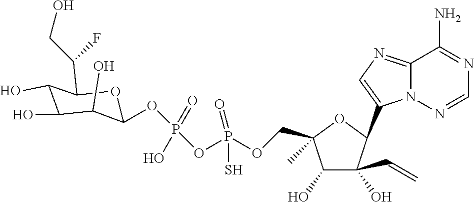 [SMILES: C=C[C@]1(O)[C@H](c2cnc3c(N)ncnn23)O[C@](C)(COP(=O)(S)OP(=O)(O)OC2OC([C@@H](F)CO)C(O)C(O)C2O)[C@H]1O] |
| Example 236C | 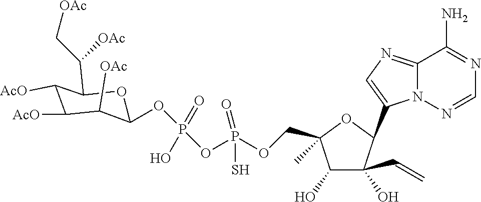 [SMILES: C=C[C@]1(O)[C@H](c2cnc3c(N)ncnn23)O[C@](C)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 236D | 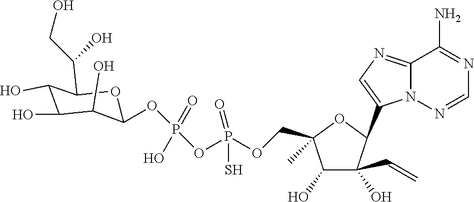 [SMILES: C=C[C@]1(O)[C@H](c2cnc3c(N)ncnn23)O[C@](C)(COP(=O)(S)OP(=O)(O)OC2OC([C@@H](O)CO)C(O)C(O)C2O)[C@H]1O] |
| Example 237A | 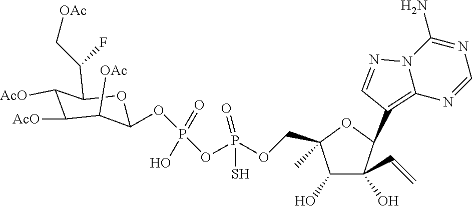 [SMILES: C=C[C@]1(O)[C@H](c2cnn3c(N)ncnc23)O[C@](C)(COP(=O)(S)OP(=O)(O)OC2OC([C@@H](F)COC(C)=O)C(OC(C)=O)C(OC(C)=O)C2OC(C)=O)[C@H]1O] |
| Example 237B | 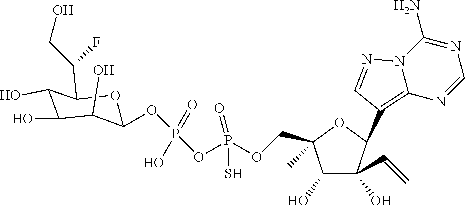 [SMILES: C=C[C@]1(O)[C@H](c2cnn3c(N)ncnc23)O[C@](C)(COP(=O)(S)OP(=O)(O)OC2OC([C@@H](F)CO)C(O)C(O)C2O)[C@H]1O] |
| Example 237C | 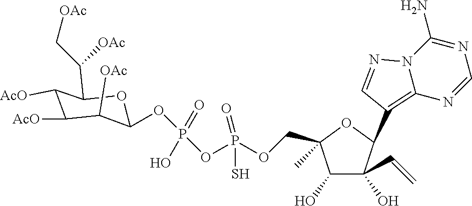 [SMILES: C=C[C@]1(O)[C@H](c2cnn3c(N)ncnc23)O[C@](C)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 237D | 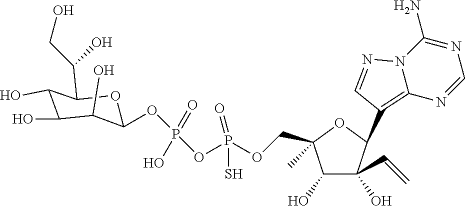 [SMILES: C=C[C@]1(O)[C@H](c2cnn3c(N)ncnc23)O[C@](C)(COP(=O)(S)OP(=O)(O)OC2OC([C@@H](O)CO)C(O)C(O)C2O)[C@H]1O] |
| Example 238A | 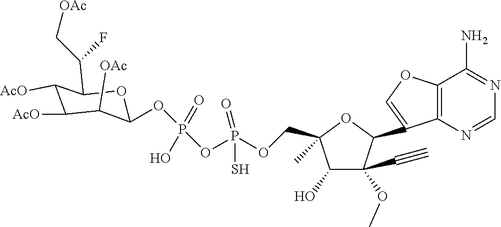 [SMILES: C#C[C@]1(OC)[C@H](c2coc3c(N)ncnc23)O[C@](C)(COP(=O)(S)OP(=O)(O)OC2OC([C@@H](F)COC(C)=O)C(OC(C)=O)C(OC(C)=O)C2OC(C)=O)[C@H]1O] |
| Example 238B | 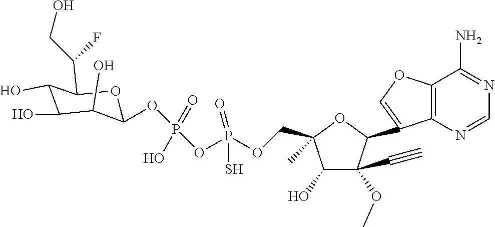 [SMILES: C#C[C@]1(OC)[C@H](c2coc3c(N)ncnc23)O[C@](C)(COP(=O)(S)OP(=O)(O)OC2OC([C@@H](F)CO)C(O)C(O)C2O)[C@H]1O] |
| Example 238C | 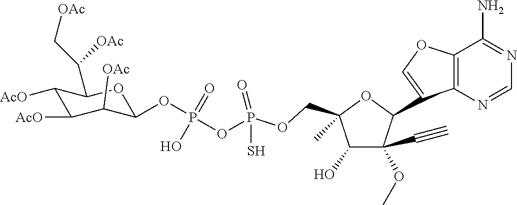 [SMILES: C#C[C@]1(OC)[C@H](c2coc3c(N)ncnc23)O[C@](C)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 238D | 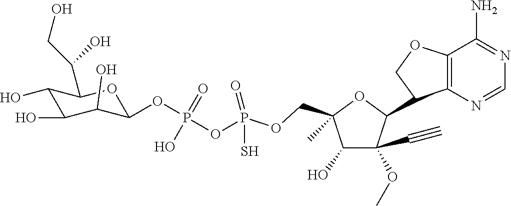 [SMILES: C#C[C@]1(OC)[C@H](C2COc3c(N)ncnc32)O[C@](C)(COP(=O)(S)OP(=O)(O)OC2OC([C@@H](O)CO)C(O)C(O)C2O)[C@H]1O] |
| Example 239A | 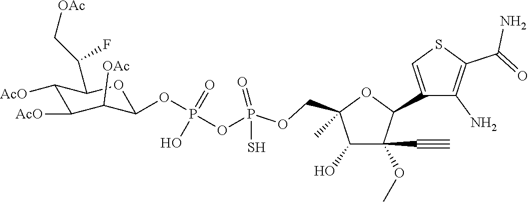 [SMILES: C#C[C@]1(OC)[C@H](c2csc(C(N)=O)c2N)O[C@](C)(COP(=O)(S)OP(=O)(O)OC2OC([C@@H](F)COC(C)=O)C(OC(C)=O)C(OC(C)=O)C2OC(C)=O)[C@H]1O] |
| Example 239B | 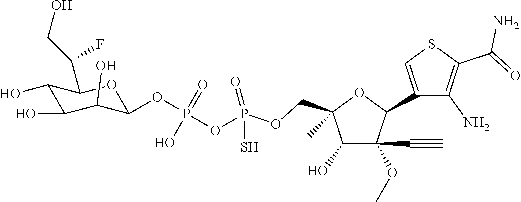 [SMILES: C#C[C@]1(OC)[C@H](c2csc(C(N)=O)c2N)O[C@](C)(COP(=O)(S)OP(=O)(O)OC2OC([C@@H](F)CO)C(O)C(O)C2O)[C@H]1O] |
| Example 239C | 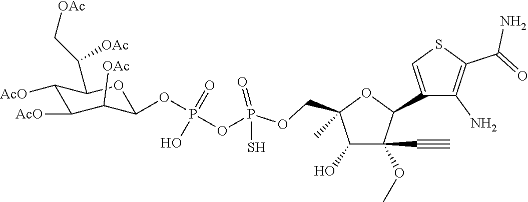 [SMILES: C#C[C@]1(OC)[C@H](c2csc(C(N)=O)c2N)O[C@](C)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 239D | 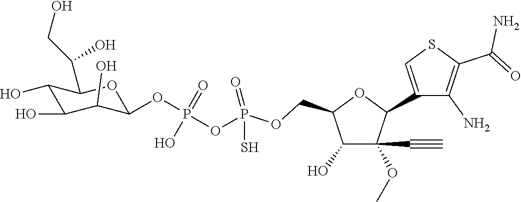 [SMILES: C#C[C@@]1(OC)[C@H](O)[C@@H](COP(=O)(S)OP(=O)(O)OC2OC([C@@H](O)CO)C(O)C(O)C2O)O[C@H]1c1csc(C(N)=O)c1N] |
| Example 240A | 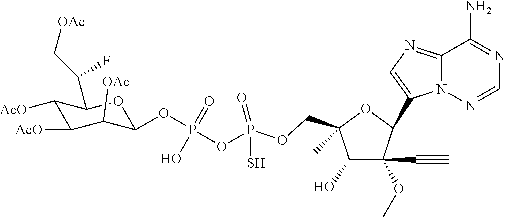 [SMILES: C#C[C@]1(OC)[C@H](c2cnc3c(N)ncnn23)O[C@](C)(COP(=O)(S)OP(=O)(O)OC2OC([C@@H](F)COC(C)=O)C(OC(C)=O)C(OC(C)=O)C2OC(C)=O)[C@H]1O] |
| Example 240B | 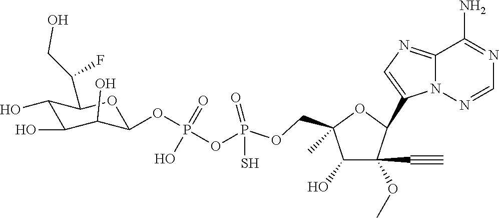 [SMILES: C#C[C@]1(OC)[C@H](c2cnc3c(N)ncnn23)O[C@](C)(COP(=O)(S)OP(=O)(O)OC2OC([C@@H](F)CO)C(O)C(O)C2O)[C@H]1O] |
| Example 240C | 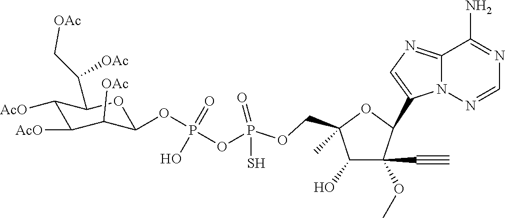 [SMILES: C#C[C@]1(OC)[C@H](c2cnc3c(N)ncnn23)O[C@](C)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 240D | 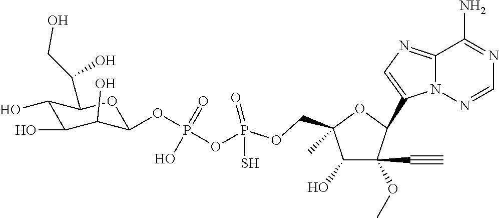 [SMILES: C#C[C@]1(OC)[C@H](c2cnc3c(N)ncnn23)O[C@](C)(COP(=O)(S)OP(=O)(O)OC2OC([C@@H](O)CO)C(O)C(O)C2O)[C@H]1O] |
| Example 241A | 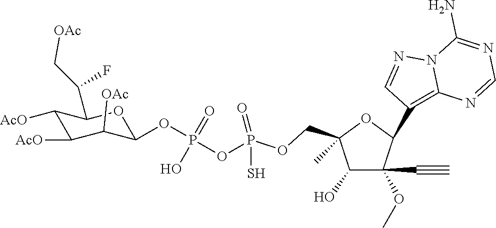 [SMILES: C#C[C@]1(OC)[C@H](c2cnn3c(N)ncnc23)O[C@](C)(COP(=O)(S)OP(=O)(O)OC2OC([C@@H](F)COC(C)=O)C(OC(C)=O)C(OC(C)=O)C2OC(C)=O)[C@H]1O] |
| Example 241B | 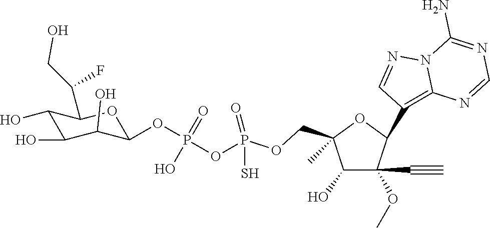 [SMILES: C#C[C@]1(OC)[C@H](c2cnn3c(N)ncnc23)O[C@](C)(COP(=O)(S)OP(=O)(O)OC2OC([C@@H](F)CO)C(O)C(O)C2O)[C@H]1O] |
| Example 241C | 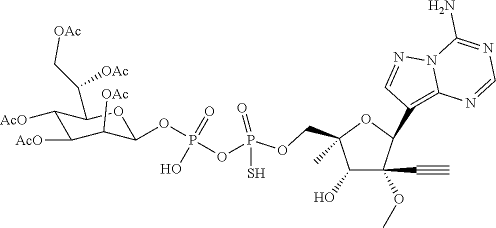 [SMILES: C#C[C@]1(OC)[C@H](c2cnn3c(N)ncnc23)O[C@](C)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 241D | 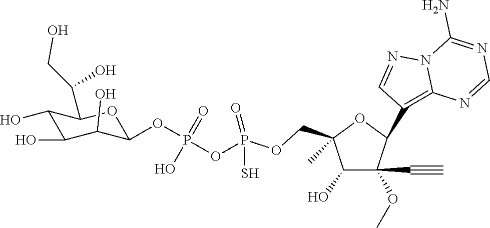 [SMILES: C#C[C@]1(OC)[C@H](c2cnn3c(N)ncnc23)O[C@](C)(COP(=O)(S)OP(=O)(O)OC2OC([C@@H](O)CO)C(O)C(O)C2O)[C@H]1O] |
| Example 242A | 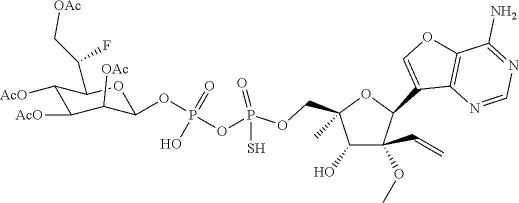 [SMILES: C=C[C@]1(OC)[C@H](c2coc3c(N)ncnc23)O[C@](C)(COP(=O)(S)OP(=O)(O)OC2OC([C@@H](F)COC(C)=O)C(OC(C)=O)C(OC(C)=O)C2OC(C)=O)[C@H]1O] |
| Example 242B | 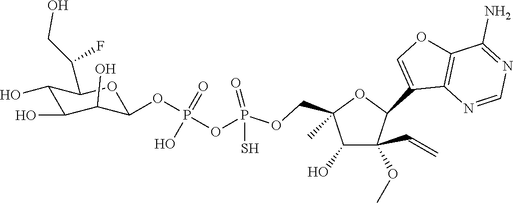 [SMILES: C=C[C@]1(OC)[C@H](c2coc3c(N)ncnc23)O[C@](C)(COP(=O)(S)OP(=O)(O)OC2OC([C@@H](F)CO)C(O)C(O)C2O)[C@H]1O] |
| Example 242C | 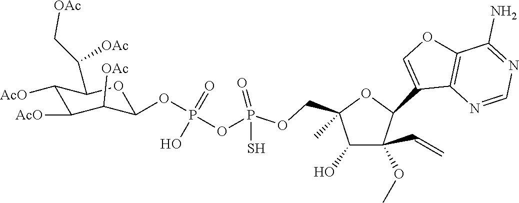 [SMILES: C=C[C@]1(OC)[C@H](c2coc3c(N)ncnc23)O[C@](C)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 242D | 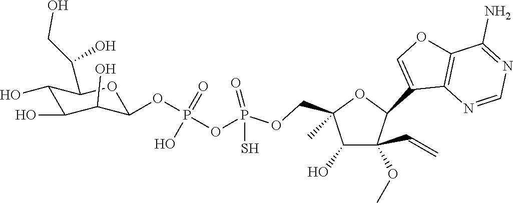 [SMILES: C=C[C@]1(OC)[C@H](c2coc3c(N)ncnc23)O[C@](C)(COP(=O)(S)OP(=O)(O)OC2OC([C@@H](O)CO)C(O)C(O)C2O)[C@H]1O] |
| Example 243A | 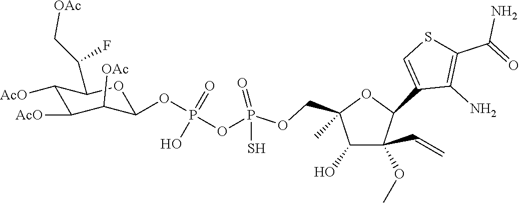 [SMILES: C=C[C@]1(OC)[C@H](c2csc(C(N)=O)c2N)O[C@](C)(COP(=O)(S)OP(=O)(O)OC2OC([C@@H](F)COC(C)=O)C(OC(C)=O)C(OC(C)=O)C2OC(C)=O)[C@H]1O] |
| Example 243B | 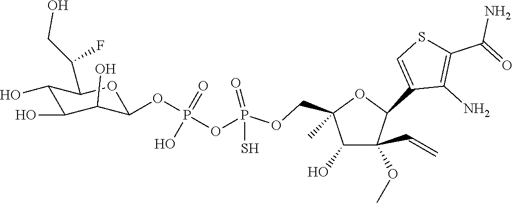 [SMILES: C=C[C@]1(OC)[C@H](c2csc(C(N)=O)c2N)O[C@](C)(COP(=O)(S)OP(=O)(O)OC2OC([C@@H](F)CO)C(O)C(O)C2O)[C@H]1O] |
| Example 243C | 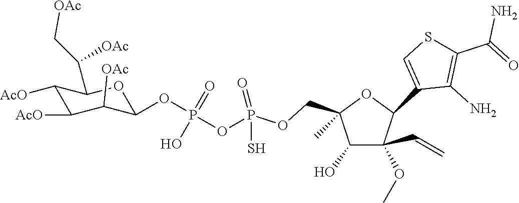 [SMILES: C=C[C@]1(OC)[C@H](c2csc(C(N)=O)c2N)O[C@](C)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 243D | 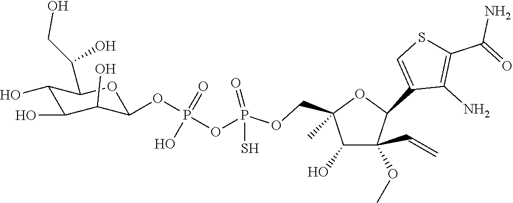 [SMILES: C=C[C@]1(OC)[C@H](c2csc(C(N)=O)c2N)O[C@](C)(COP(=O)(S)OP(=O)(O)OC2OC([C@@H](O)CO)C(O)C(O)C2O)[C@H]1O] |
| Example 244A | 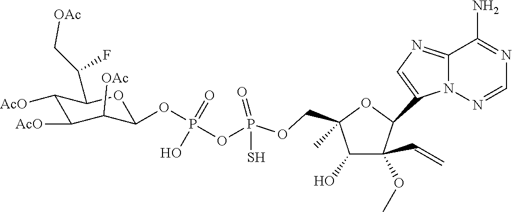 [SMILES: C=C[C@]1(OC)[C@H](c2cnc3c(N)ncnn23)O[C@](C)(COP(=O)(S)OP(=O)(O)OC2OC([C@@H](F)COC(C)=O)C(OC(C)=O)C(OC(C)=O)C2OC(C)=O)[C@H]1O] |
| Example 244B | 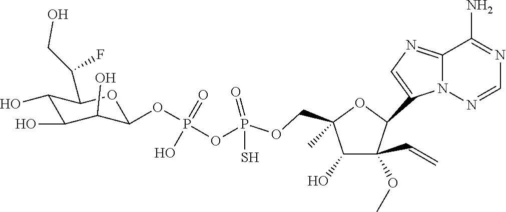 [SMILES: C=C[C@]1(OC)[C@H](c2cnc3c(N)ncnn23)O[C@](C)(COP(=O)(S)OP(=O)(O)OC2OC([C@@H](F)CO)C(O)C(O)C2O)[C@H]1O] |
| Example 244C | 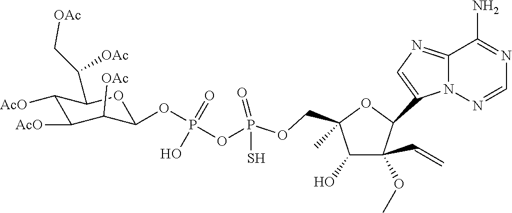 [SMILES: C=C[C@]1(OC)[C@H](c2cnc3c(N)ncnn23)O[C@](C)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 244D | 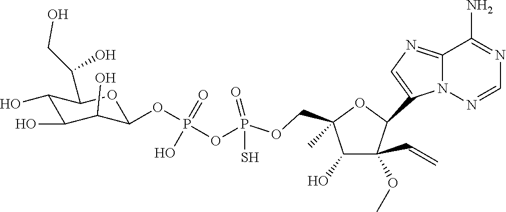 [SMILES: C=C[C@]1(OC)[C@H](c2cnc3c(N)ncnn23)O[C@](C)(COP(=O)(S)OP(=O)(O)OC2OC([C@@H](O)CO)C(O)C(O)C2O)[C@H]1O] |
| Example 245A | 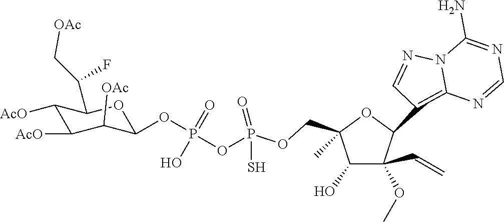 [SMILES: C=C[C@]1(OC)[C@H](c2cnn3c(N)ncnc23)O[C@](C)(COP(=O)(S)OP(=O)(O)OC2OC([C@@H](F)COC(C)=O)C(OC(C)=O)C(OC(C)=O)C2OC(C)=O)[C@H]1O] |
| Example 245B | 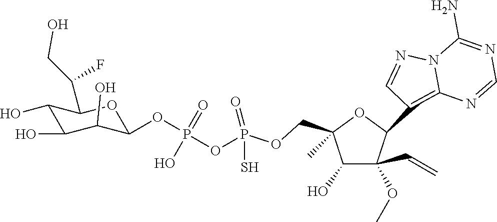 [SMILES: C=C[C@]1(OC)[C@H](c2cnn3c(N)ncnc23)O[C@](C)(COP(=O)(S)OP(=O)(O)OC2OC([C@@H](F)CO)C(O)C(O)C2O)[C@H]1O] |
| Example 245C | 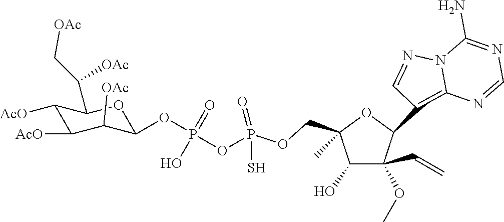 [SMILES: C=C[C@]1(OC)[C@H](c2cnn3c(N)ncnc23)O[C@](C)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 245D | 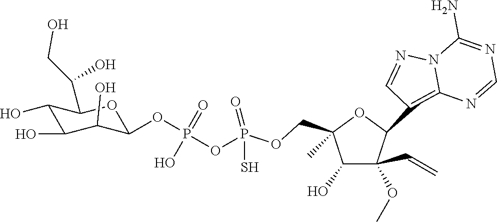 [SMILES: C=C[C@]1(OC)[C@H](c2cnn3c(N)ncnc23)O[C@](C)(COP(=O)(S)OP(=O)(O)OC2OC([C@@H](O)CO)C(O)C(O)C2O)[C@H]1O] |
| Example 246A | 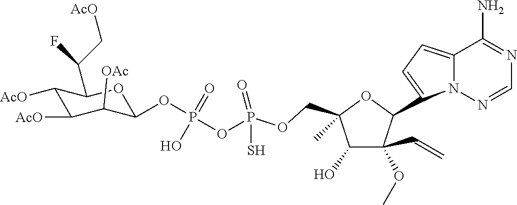 [SMILES: C=C[C@]1(OC)[C@H](c2ccc3c(N)ncnn23)O[C@](C)(COP(=O)(S)OP(=O)(O)OC2OC([C@@H](F)COC(C)=O)C(OC(C)=O)C(OC(C)=O)C2OC(C)=O)[C@H]1O] |
| Example 246B | 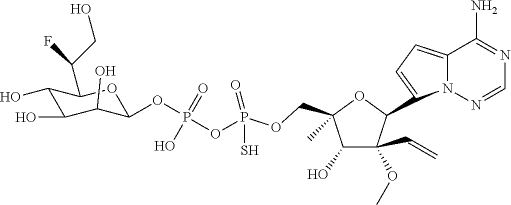 [SMILES: C=C[C@]1(OC)[C@H](c2ccc3c(N)ncnn23)O[C@](C)(COP(=O)(S)OP(=O)(O)OC2OC([C@@H](F)CO)C(O)C(O)C2O)[C@H]1O] |
| Example 246C | 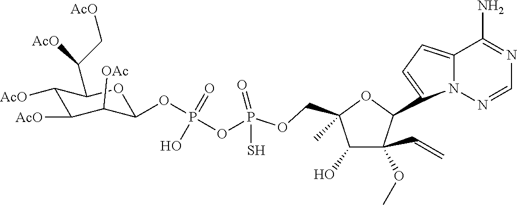 [SMILES: C=C[C@]1(OC)[C@H](c2ccc3c(N)ncnn23)O[C@](C)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 246D | 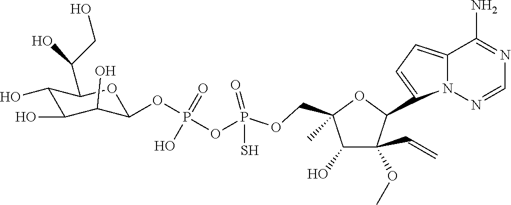 [SMILES: C=C[C@]1(OC)[C@H](c2ccc3c(N)ncnn23)O[C@](C)(COP(=O)(S)OP(=O)(O)OC2OC([C@@H](O)CO)C(O)C(O)C2O)[C@H]1O] |
| Example 247A | 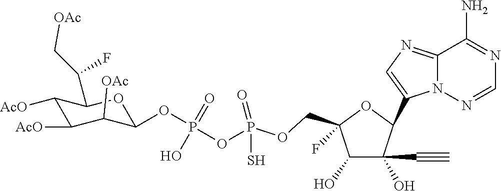 [SMILES: C#C[C@]1(O)[C@H](c2cnc3c(N)ncnn23)O[C@](F)(COP(=O)(S)OP(=O)(O)OC2OC([C@@H](F)COC(C)=O)C(OC(C)=O)C(OC(C)=O)C2OC(C)=O)[C@H]1O] |
| Example 247B | 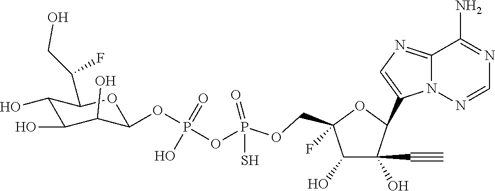 [SMILES: C#C[C@]1(O)[C@H](c2cnc3c(N)ncnn23)O[C@](F)(COP(=O)(S)OP(=O)(O)OC2OC([C@@H](F)CO)C(O)C(O)C2O)[C@H]1O] |
| Example 247C | 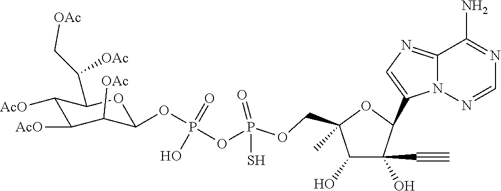 [SMILES: C#C[C@]1(O)[C@H](c2cnc3c(N)ncnn23)O[C@](C)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 247D | 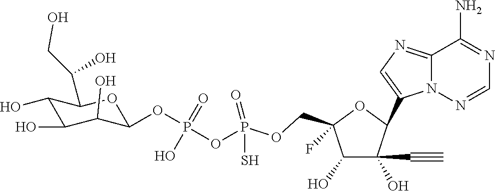 [SMILES: C#C[C@]1(O)[C@H](c2cnc3c(N)ncnn23)O[C@](F)(COP(=O)(S)OP(=O)(O)OC2OC([C@@H](O)CO)C(O)C(O)C2O)[C@H]1O] |
| Example 248A | 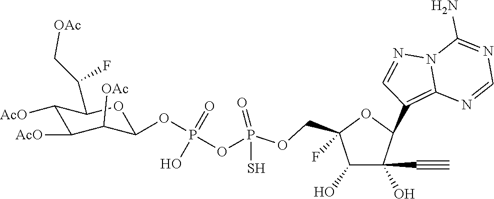 [SMILES: C#C[C@]1(O)[C@H](c2cnn3c(N)ncnc23)O[C@](F)(COP(=O)(S)OP(=O)(O)OC2OC([C@@H](F)COC(C)=O)C(OC(C)=O)C(OC(C)=O)C2OC(C)=O)[C@H]1O] |
| Example 248B | 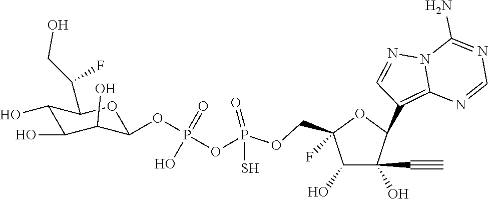 [SMILES: C#C[C@]1(O)[C@H](c2cnn3c(N)ncnc23)O[C@](F)(COP(=O)(S)OP(=O)(O)OC2OC([C@@H](F)CO)C(O)C(O)C2O)[C@H]1O] |
| Example 248C | 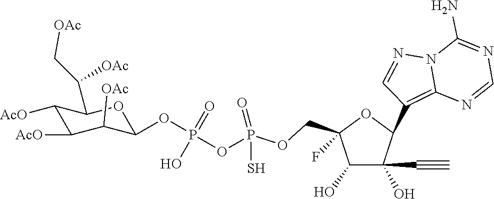 [SMILES: C#C[C@]1(O)[C@H](c2cnn3c(N)ncnc23)O[C@](F)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 248D | 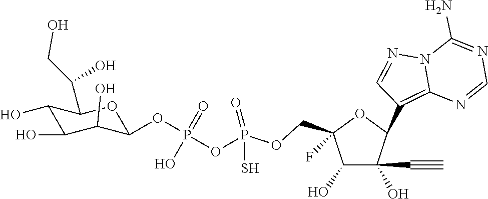 [SMILES: C#C[C@]1(O)[C@H](c2cnn3c(N)ncnc23)O[C@](F)(COP(=O)(S)OP(=O)(O)OC2OC([C@@H](O)CO)C(O)C(O)C2O)[C@H]1O] |
| Example 249A | 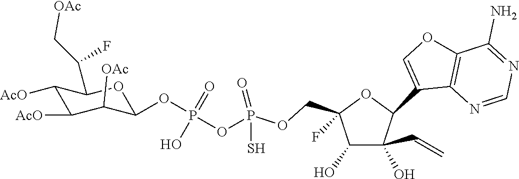 [SMILES: C=C[C@]1(O)[C@H](c2coc3c(N)ncnc23)O[C@](F)(COP(=O)(S)OP(=O)(O)OC2OC([C@@H](F)COC(C)=O)C(OC(C)=O)C(OC(C)=O)C2OC(C)=O)[C@H]1O] |
| Example 249B | 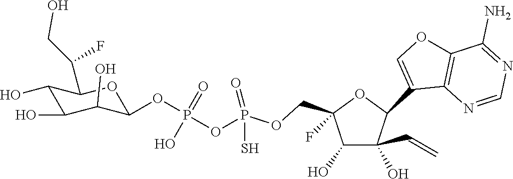 [SMILES: C=C[C@]1(O)[C@H](c2coc3c(N)ncnc23)O[C@](F)(COP(=O)(S)OP(=O)(O)OC2OC([C@@H](F)CO)C(O)C(O)C2O)[C@H]1O] |
| Example 249C | 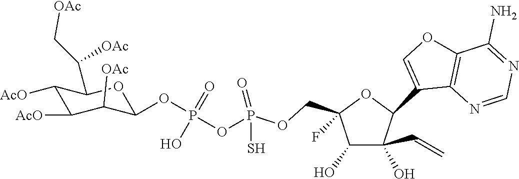 [SMILES: C=C[C@]1(O)[C@H](c2coc3c(N)ncnc23)O[C@](F)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 249D | 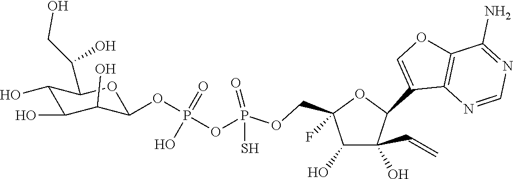 [SMILES: C=C[C@]1(O)[C@H](c2coc3c(N)ncnc23)O[C@](F)(COP(=O)(S)OP(=O)(O)OC2OC([C@@H](O)CO)C(O)C(O)C2O)[C@H]1O] |
| Example 250A | 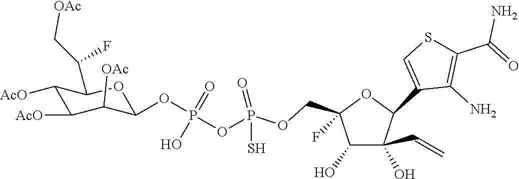 [SMILES: C=C[C@]1(O)[C@H](c2csc(C(N)=O)c2N)O[C@](F)(COP(=O)(S)OP(=O)(O)OC2OC([C@@H](F)COC(C)=O)C(OC(C)=O)C(OC(C)=O)C2OC(C)=O)[C@H]1O] |
| Example 250B | 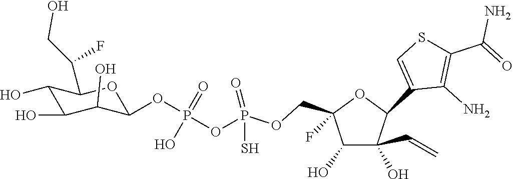 [SMILES: C=C[C@]1(O)[C@H](c2csc(C(N)=O)c2N)O[C@](F)(COP(=O)(S)OP(=O)(O)OC2OC([C@@H](F)CO)C(O)C(O)C2O)[C@H]1O] |
| Example 250C | 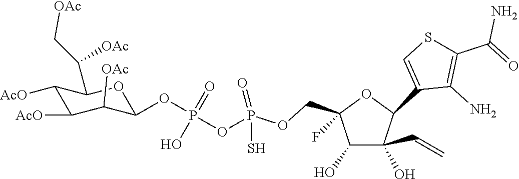 [SMILES: C=C[C@]1(O)[C@H](c2csc(C(N)=O)c2N)O[C@](F)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 250D | 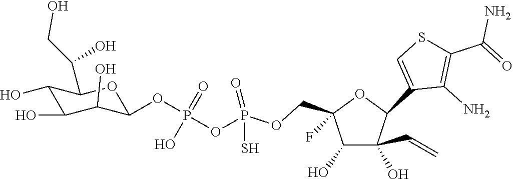 [SMILES: C=C[C@]1(O)[C@H](c2csc(C(N)=O)c2N)O[C@](F)(COP(=O)(S)OP(=O)(O)OC2OC([C@@H](O)CO)C(O)C(O)C2O)[C@H]1O] |
| Example 251A | 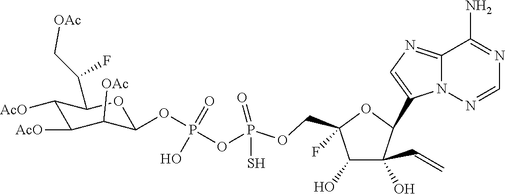 [SMILES: C=C[C@]1(O)[C@H](c2cnc3c(N)ncnn23)O[C@](F)(COP(=O)(S)OP(=O)(O)OC2OC([C@@H](F)COC(C)=O)C(OC(C)=O)C(OC(C)=O)C2OC(C)=O)[C@H]1O] |
| Example 251B | 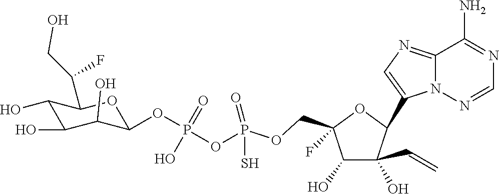 [SMILES: C=C[C@]1(O)[C@H](c2cnc3c(N)ncnn23)O[C@](F)(COP(=O)(S)OP(=O)(O)OC2OC([C@@H](F)CO)C(O)C(O)C2O)[C@H]1O] |
| Example 251C | 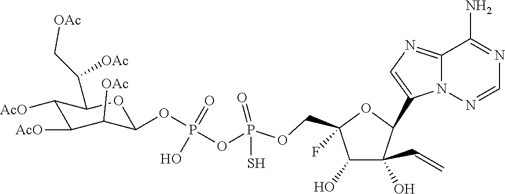 [SMILES: C=C[C@]1(O)[C@H](c2cnc3c(N)ncnn23)O[C@](F)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 251D | 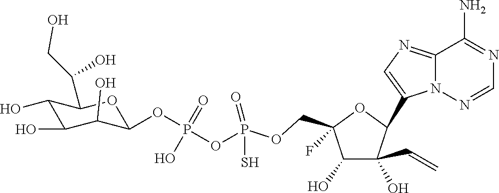 [SMILES: C=C[C@]1(O)[C@H](c2cnc3c(N)ncnn23)O[C@](F)(COP(=O)(S)OP(=O)(O)OC2OC([C@@H](O)CO)C(O)C(O)C2O)[C@H]1O] |
| Example 252A | 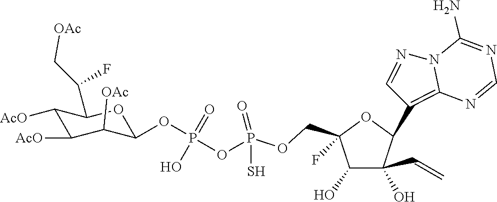 [SMILES: C=C[C@]1(O)[C@H](c2cnn3c(N)ncnc23)O[C@](F)(COP(=O)(S)OP(=O)(O)OC2OC([C@@H](F)COC(C)=O)C(OC(C)=O)C(OC(C)=O)C2OC(C)=O)[C@H]1O] |
| Example 252B | 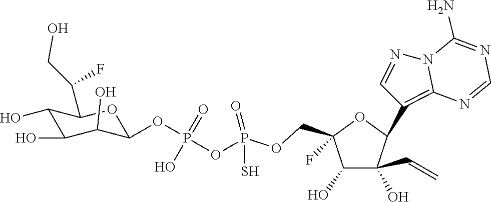 [SMILES: C=C[C@]1(O)[C@H](c2cnn3c(N)ncnc23)O[C@](F)(COP(=O)(S)OP(=O)(O)OC2OC([C@@H](F)CO)C(O)C(O)C2O)[C@H]1O] |
| Example 252C | 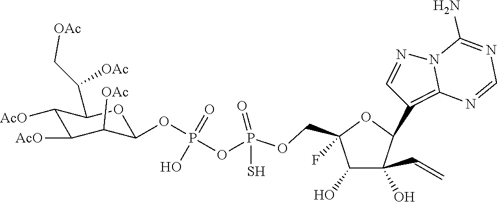 [SMILES: C=C[C@]1(O)[C@H](c2cnn3c(N)ncnc23)O[C@](F)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 252D | 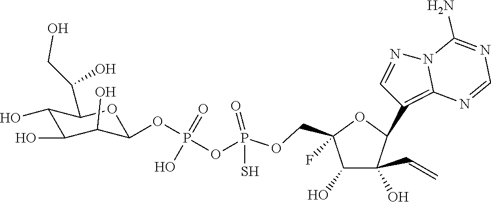 [SMILES: C=C[C@]1(O)[C@H](c2cnn3c(N)ncnc23)O[C@](F)(COP(=O)(S)OP(=O)(O)OC2OC([C@@H](O)CO)C(O)C(O)C2O)[C@H]1O] |
| Example 253A | 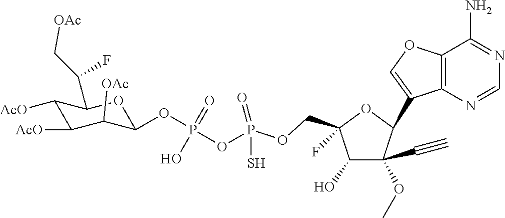 [SMILES: C#C[C@]1(OC)[C@H](c2coc3c(N)ncnc23)O[C@](F)(COP(=O)(S)OP(=O)(O)OC2OC([C@@H](F)COC(C)=O)C(OC(C)=O)C(OC(C)=O)C2OC(C)=O)[C@H]1O] |
| Example 253B | 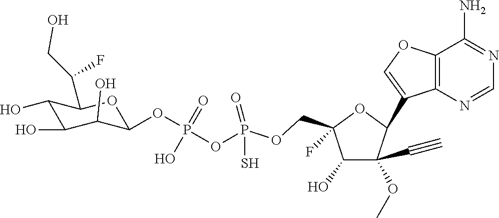 [SMILES: C#C[C@]1(OC)[C@H](c2coc3c(N)ncnc23)O[C@](F)(COP(=O)(S)OP(=O)(O)OC2OC([C@@H](F)CO)C(O)C(O)C2O)[C@H]1O] |
| Example 253C | 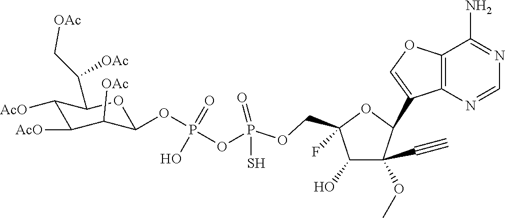 [SMILES: C#C[C@]1(OC)[C@H](c2coc3c(N)ncnc23)O[C@](F)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 253D | 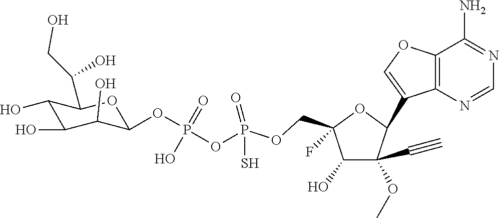 [SMILES: C#C[C@]1(OC)[C@H](c2coc3c(N)ncnc23)O[C@](F)(COP(=O)(S)OP(=O)(O)OC2OC([C@@H](O)CO)C(O)C(O)C2O)[C@H]1O] |
| Example 254A | 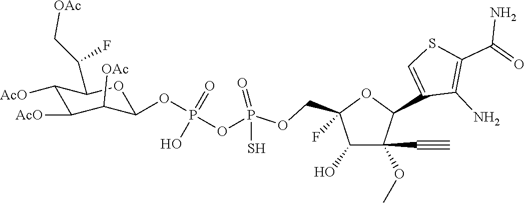 [SMILES: C#C[C@]1(OC)[C@H](c2csc(C(N)=O)c2N)O[C@](F)(COP(=O)(S)OP(=O)(O)OC2OC([C@@H](F)COC(C)=O)C(OC(C)=O)C(OC(C)=O)C2OC(C)=O)[C@H]1O] |
| Example 254B | 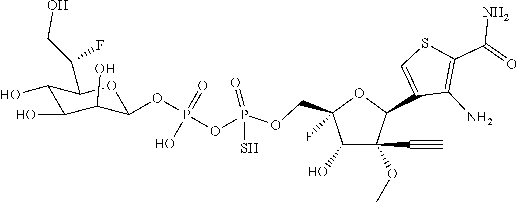 [SMILES: C#C[C@]1(OC)[C@H](c2csc(C(N)=O)c2N)O[C@](F)(COP(=O)(S)OP(=O)(O)OC2OC([C@@H](F)CO)C(O)C(O)C2O)[C@H]1O] |
| Example 254C | 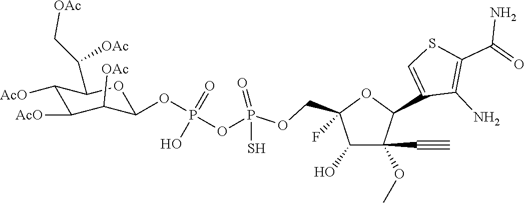 [SMILES: C#C[C@]1(OC)[C@H](c2csc(C(N)=O)c2N)O[C@](F)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 254D | 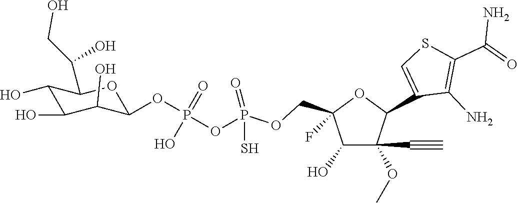 [SMILES: C#C[C@]1(OC)[C@H](c2csc(C(N)=O)c2N)O[C@](F)(COP(=O)(S)OP(=O)(O)OC2OC([C@@H](O)CO)C(O)C(O)C2O)[C@H]1O] |
| Example 255A | 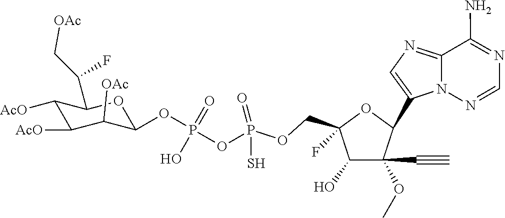 [SMILES: C#C[C@]1(OC)[C@H](c2cnc3c(N)ncnn23)O[C@](F)(COP(=O)(S)OP(=O)(O)OC2OC([C@@H](F)COC(C)=O)C(OC(C)=O)C(OC(C)=O)C2OC(C)=O)[C@H]1O] |
| Example 255B | 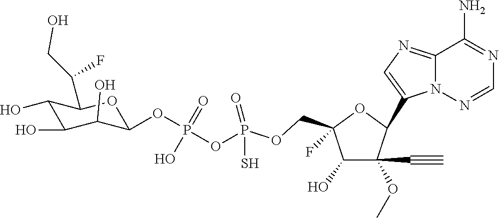 [SMILES: C#C[C@]1(OC)[C@H](c2cnc3c(N)ncnn23)O[C@](F)(COP(=O)(S)OP(=O)(O)OC2OC([C@@H](F)CO)C(O)C(O)C2O)[C@H]1O] |
| Example 255C | 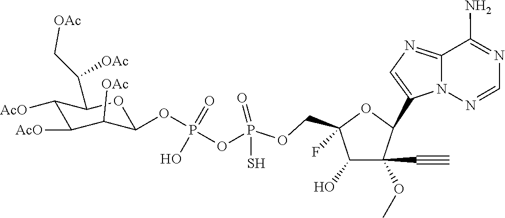 [SMILES: C#C[C@]1(OC)[C@H](c2cnc3c(N)ncnn23)O[C@](F)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 255D | 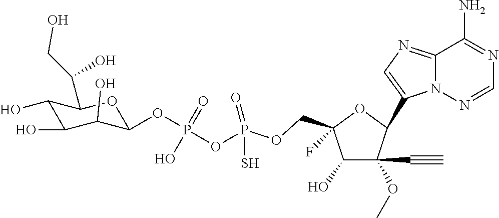 [SMILES: C#C[C@]1(OC)[C@H](c2cnc3c(N)ncnn23)O[C@](F)(COP(=O)(S)OP(=O)(O)OC2OC([C@@H](O)CO)C(O)C(O)C2O)[C@H]1O] |
| Example 256A | 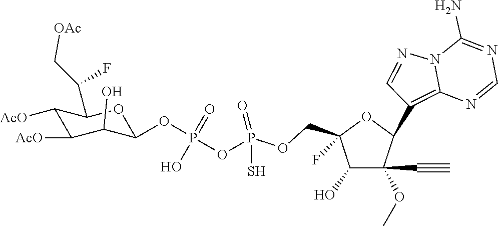 [SMILES: C#C[C@]1(OC)[C@H](c2cnn3c(N)ncnc23)O[C@](F)(COP(=O)(S)OP(=O)(O)OC2OC([C@@H](F)COC(C)=O)C(OC(C)=O)C(OC(C)=O)C2O)[C@H]1O] |
| Example 256B | 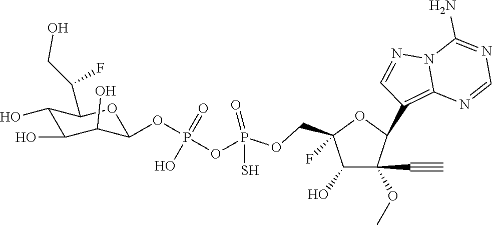 [SMILES: C#C[C@]1(OC)[C@H](c2cnn3c(N)ncnc23)O[C@](F)(COP(=O)(S)OP(=O)(O)OC2OC([C@@H](F)CO)C(O)C(O)C2O)[C@H]1O] |
| Example 256C | 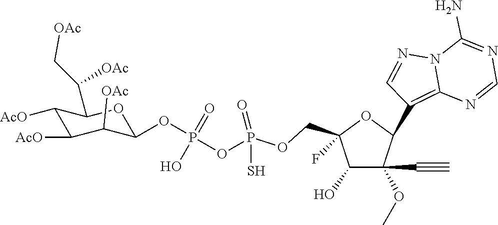 [SMILES: C#C[C@]1(OC)[C@H](c2cnn3c(N)ncnc23)O[C@](F)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 256D | 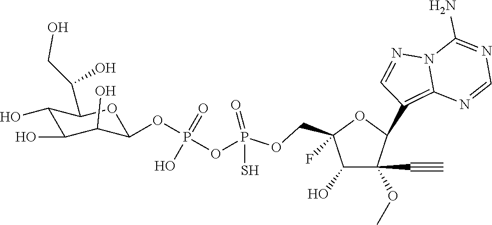 [SMILES: C#C[C@]1(OC)[C@H](c2cnn3c(N)ncnc23)O[C@](F)(COP(=O)(S)OP(=O)(O)OC2OC([C@@H](O)CO)C(O)C(O)C2O)[C@H]1O] |
| Example 257A | 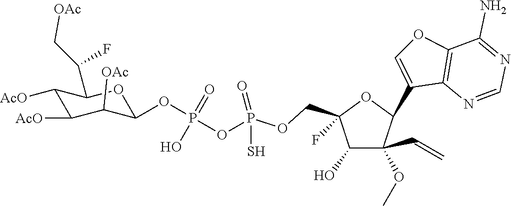 [SMILES: C=C[C@]1(OC)[C@H](c2coc3c(N)ncnc23)O[C@](F)(COP(=O)(S)OP(=O)(O)OC2OC([C@@H](F)COC(C)=O)C(OC(C)=O)C(OC(C)=O)C2OC(C)=O)[C@H]1O] |
| Example 257B | 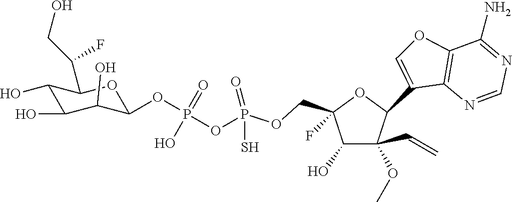 [SMILES: C=C[C@]1(OC)[C@H](c2coc3c(N)ncnc23)O[C@](F)(COP(=O)(S)OP(=O)(O)OC2OC([C@@H](F)CO)C(O)C(O)C2O)[C@H]1O] |
| Example 257C | 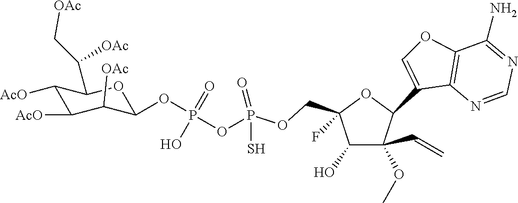 [SMILES: C=C[C@]1(OC)[C@H](c2coc3c(N)ncnc23)O[C@](F)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 257D | 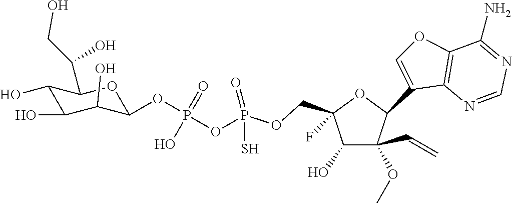 [SMILES: C=C[C@]1(OC)[C@H](c2coc3c(N)ncnc23)O[C@](F)(COP(=O)(S)OP(=O)(O)OC2OC([C@@H](O)CO)C(O)C(O)C2O)[C@H]1O] |
| Example 258A | 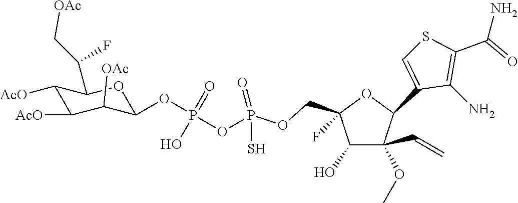 [SMILES: C=C[C@]1(OC)[C@H](c2csc(C(N)=O)c2N)O[C@](F)(COP(=O)(S)OP(=O)(O)OC2OC([C@@H](F)COC(C)=O)C(OC(C)=O)C(OC(C)=O)C2OC(C)=O)[C@H]1O] |
| Example 258B | 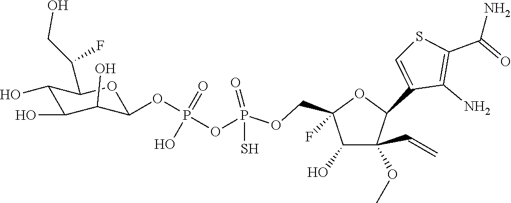 [SMILES: C=C[C@]1(OC)[C@H](c2csc(C(N)=O)c2N)O[C@](F)(COP(=O)(S)OP(=O)(O)OC2OC([C@@H](F)CO)C(O)C(O)C2O)[C@H]1O] |
| Example 258C | 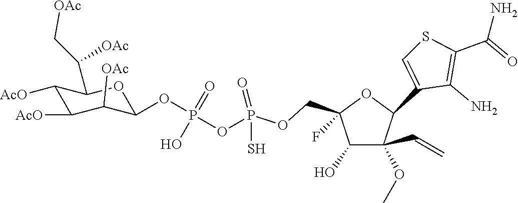 [SMILES: C=C[C@]1(OC)[C@H](c2csc(C(N)=O)c2N)O[C@](F)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 258D | 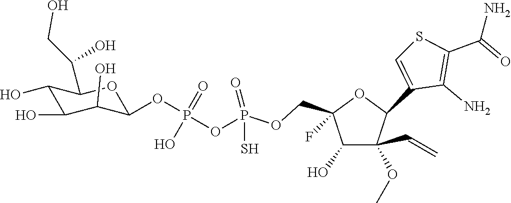 [SMILES: C=C[C@]1(OC)[C@H](c2csc(C(N)=O)c2N)O[C@](F)(COP(=O)(S)OP(=O)(O)OC2OC([C@@H](O)CO)C(O)C(O)C2O)[C@H]1O] |
| Example 259A | 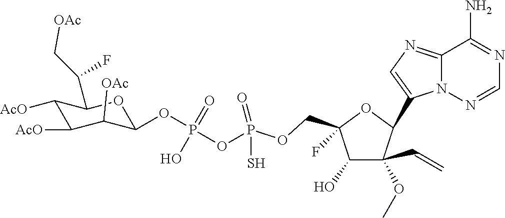 [SMILES: C=C[C@]1(OC)[C@H](c2cnc3c(N)ncnn23)O[C@](F)(COP(=O)(S)OP(=O)(O)OC2OC([C@@H](F)COC(C)=O)C(OC(C)=O)C(OC(C)=O)C2OC(C)=O)[C@H]1O] |
| Example 259B | 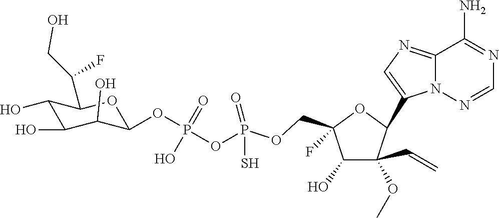 [SMILES: C=C[C@]1(OC)[C@H](c2cnc3c(N)ncnn23)O[C@](F)(COP(=O)(S)OP(=O)(O)OC2OC([C@@H](F)CO)C(O)C(O)C2O)[C@H]1O] |
| Example 259C | 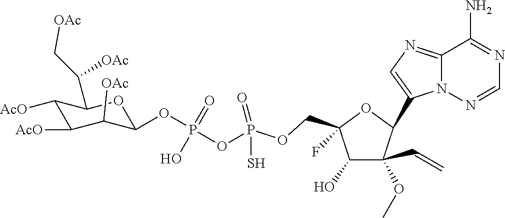 [SMILES: C=C[C@]1(OC)[C@H](c2cnc3c(N)ncnn23)O[C@](F)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 259D | 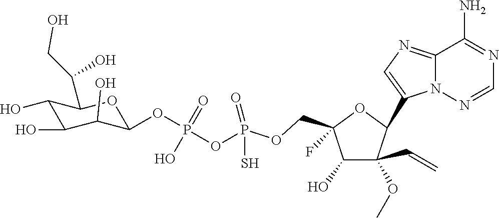 [SMILES: C=C[C@]1(OC)[C@H](c2cnc3c(N)ncnn23)O[C@](F)(COP(=O)(S)OP(=O)(O)OC2OC([C@@H](O)CO)C(O)C(O)C2O)[C@H]1O] |
| Example 260A | 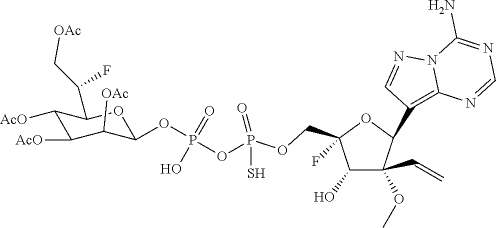 [SMILES: C=C[C@]1(OC)[C@H](c2cnn3c(N)ncnc23)O[C@](F)(COP(=O)(S)OP(=O)(O)OC2OC([C@@H](F)COC(C)=O)C(OC(C)=O)C(OC(C)=O)C2OC(C)=O)[C@H]1O] |
| Example 260B | 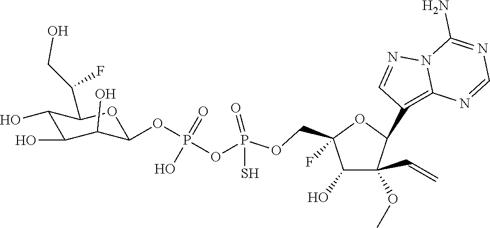 [SMILES: C=C[C@]1(OC)[C@H](c2cnn3c(N)ncnc23)O[C@](F)(COP(=O)(S)OP(=O)(O)OC2OC([C@@H](F)CO)C(O)C(O)C2O)[C@H]1O] |
| Example 260C | 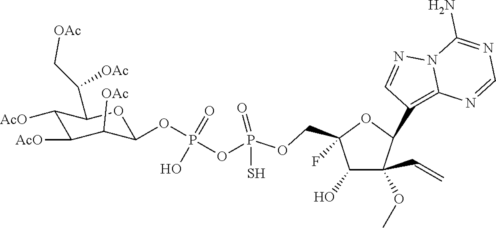 [SMILES: C=C[C@]1(OC)[C@H](c2cnn3c(N)ncnc23)O[C@](F)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 260D | 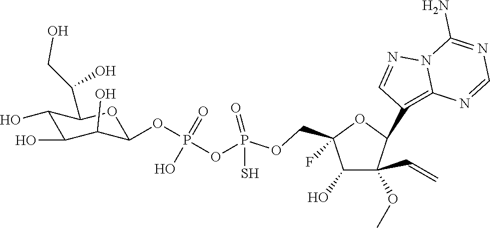 [SMILES: C=C[C@]1(OC)[C@H](c2cnn3c(N)ncnc23)O[C@](F)(COP(=O)(S)OP(=O)(O)OC2OC([C@@H](O)CO)C(O)C(O)C2O)[C@H]1O] |
| Example 261A | 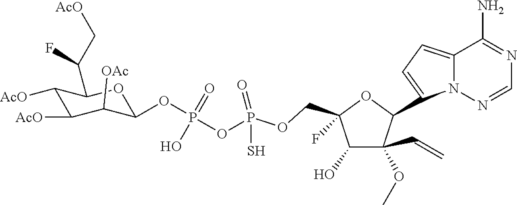 [SMILES: C=C[C@]1(OC)[C@H](c2ccc3c(N)ncnn23)O[C@](F)(COP(=O)(S)OP(=O)(O)OC2OC([C@@H](F)COC(C)=O)C(OC(C)=O)C(OC(C)=O)C2OC(C)=O)[C@H]1O] |
| Example 261B | 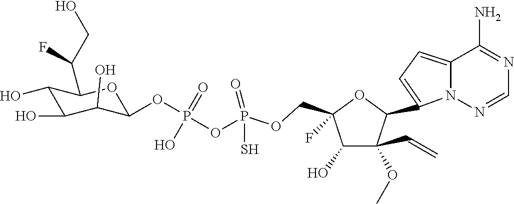 [SMILES: C=C[C@]1(OC)[C@H](c2ccc3c(N)ncnn23)O[C@](F)(COP(=O)(S)OP(=O)(O)OC2OC([C@@H](F)CO)C(O)C(O)C2O)[C@H]1O] |
| Example 261C | 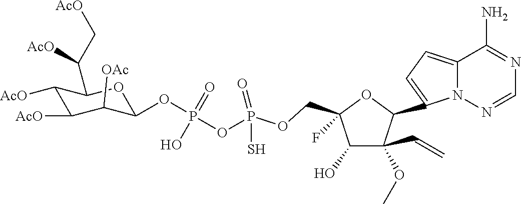 [SMILES: C=C[C@]1(OC)[C@H](c2ccc3c(N)ncnn23)O[C@](F)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 261D | 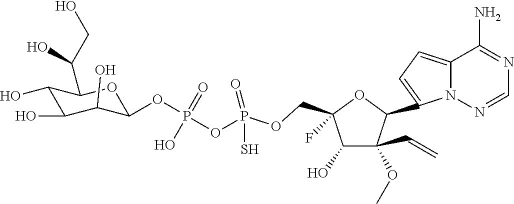 [SMILES: C=C[C@]1(OC)[C@H](c2ccc3c(N)ncnn23)O[C@](F)(COP(=O)(S)OP(=O)(O)OC2OC([C@@H](O)CO)C(O)C(O)C2O)[C@H]1O] |
| Example 262C | 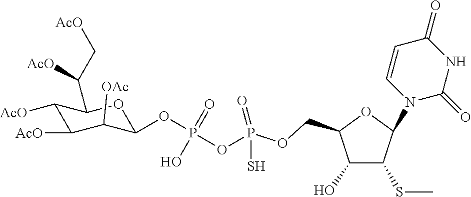 [SMILES: CS[C@@H]1[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1ccc(=O)[nH]c1=O] |
| Example 262D | 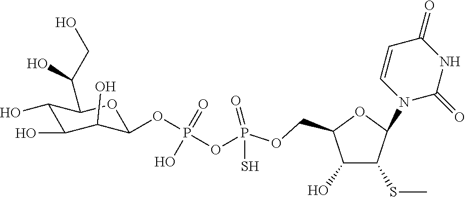 [SMILES: CS[C@@H]1[C@H](O)[C@@H](COP(=O)(S)OP(=O)(O)OC2OC([C@@H](O)CO)C(O)C(O)C2O)O[C@H]1n1ccc(=O)[nH]c1=O] |
| Example 263C | 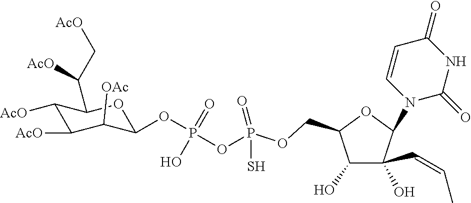 [SMILES: C/C=C\[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1ccc(=O)[nH]c1=O] |
| Example 264A | 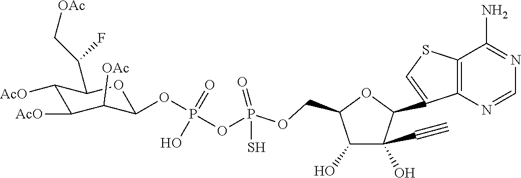 [SMILES: C#C[C@@]1(O)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1csc2c(N)ncnc12] |
| Example 264B | 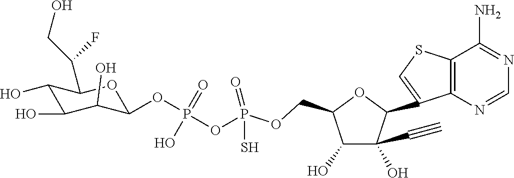 [SMILES: C#C[C@@]1(O)[C@H](O)[C@@H](COP(=O)(S)OP(=O)(O)OC2OC([C@@H](F)CO)C(O)C(O)C2O)O[C@H]1c1csc2c(N)ncnc12] |
| Example 264C | 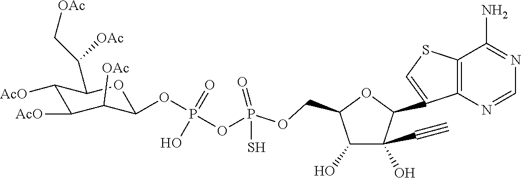 [SMILES: C#C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1csc2c(N)ncnc12] |
| Example 264D | 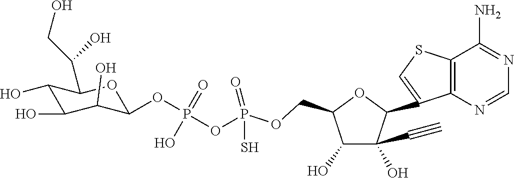 [SMILES: C#C[C@@]1(O)[C@H](O)[C@@H](COP(=O)(S)OP(=O)(O)OC2OC([C@@H](O)CO)C(O)C(O)C2O)O[C@H]1c1csc2c(N)ncnc12] |
| Example 265A | 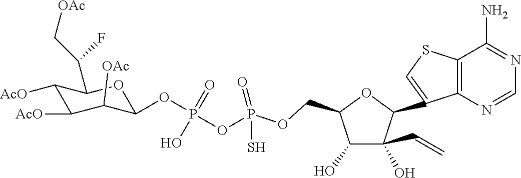 [SMILES: C=C[C@@]1(O)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1csc2c(N)ncnc12] |
| Example 265B | 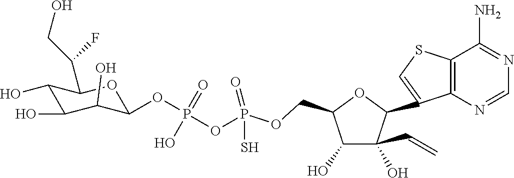 [SMILES: C=C[C@@]1(O)[C@H](O)[C@@H](COP(=O)(S)OP(=O)(O)OC2OC([C@@H](F)CO)C(O)C(O)C2O)O[C@H]1c1csc2c(N)ncnc12] |
| Example 265C | 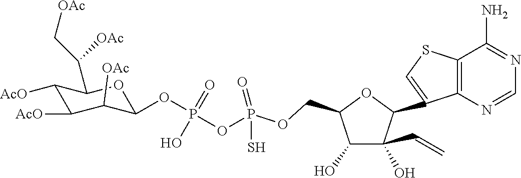 [SMILES: C=C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1csc2c(N)ncnc12] |
| Example 265D | 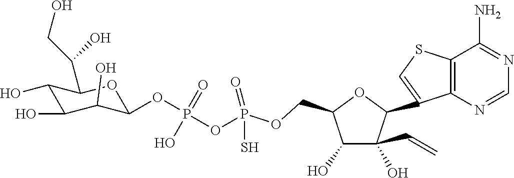 [SMILES: C=C[C@@]1(O)[C@H](O)[C@@H](COP(=O)(S)OP(=O)(O)OC2OC([C@@H](O)CO)C(O)C(O)C2O)O[C@H]1c1csc2c(N)ncnc12] |
| Example 266A | 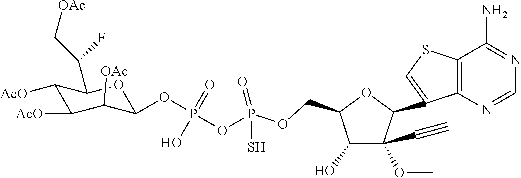 [SMILES: C#C[C@@]1(OC)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1csc2c(N)ncnc12] |
| Example 266B | 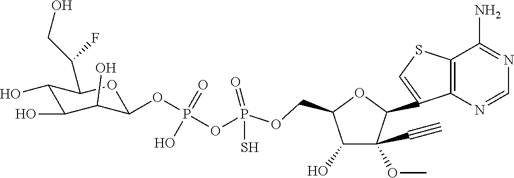 [SMILES: C#C[C@@]1(OC)[C@H](O)[C@@H](COP(=O)(S)OP(=O)(O)OC2OC([C@@H](F)CO)C(O)C(O)C2O)O[C@H]1c1csc2c(N)ncnc12] |
| Example 266C | 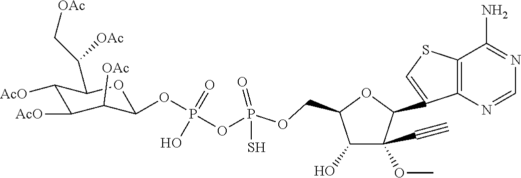 [SMILES: C#C[C@@]1(OC)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1csc2c(N)ncnc12] |
| Example 266D | 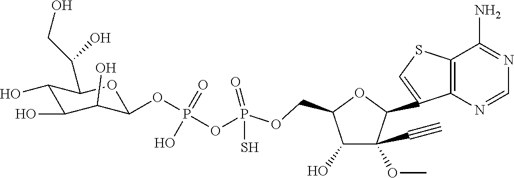 [SMILES: C#C[C@@]1(OC)[C@H](O)[C@@H](COP(=O)(S)OP(=O)(O)OC2OC([C@@H](O)CO)C(O)C(O)C2O)O[C@H]1c1csc2c(N)ncnc12] |
| Example 267A | 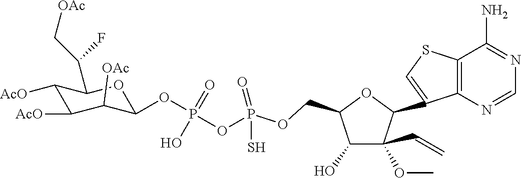 [SMILES: C=C[C@@]1(OC)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1csc2c(N)ncnc12] |
| Example 267B | 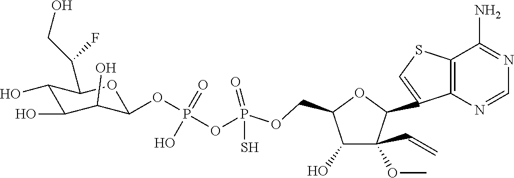 [SMILES: C=C[C@@]1(OC)[C@H](O)[C@@H](COP(=O)(S)OP(=O)(O)OC2OC([C@@H](F)CO)C(O)C(O)C2O)O[C@H]1c1csc2c(N)ncnc12] |
| Example 267C | 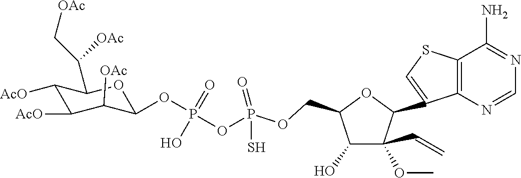 [SMILES: C=C[C@@]1(OC)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1csc2c(N)ncnc12] |
| Example 267D | 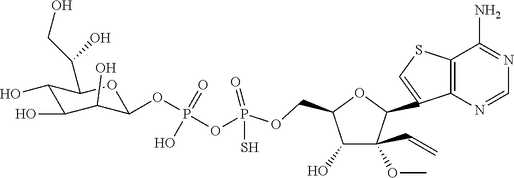 [SMILES: C=C[C@@]1(OC)[C@H](O)[C@@H](COP(=O)(S)OP(=O)(O)OC2OC([C@@H](O)CO)C(O)C(O)C2O)O[C@H]1c1csc2c(N)ncnc12] |
| Example 268A | 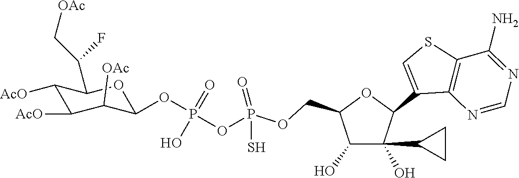 [SMILES: CC(=O)OC[C@H](F)C1OC(OP(=O)(O)OP(=O)(S)OC[C@H]2O[C@@H](c3csc4c(N)ncnc34)[C@@](O)(C3CC3)[C@@H]2O)C(OC(C)=O)C(OC(C)=O)C1OC(C)=O] |
| Example 268B | 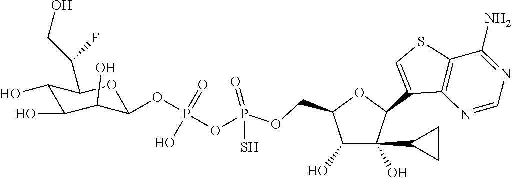 [SMILES: Nc1ncnc2c([C@@H]3O[C@H](COP(=O)(S)OP(=O)(O)OC4OC([C@@H](F)CO)C(O)C(O)C4O)[C@@H](O)[C@]3(O)C3CC3)csc12] |
| Example 268C | 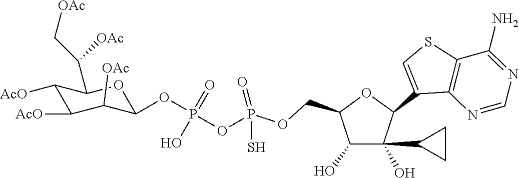 [SMILES: CC(=O)OC[C@H](OC(C)=O)C1OC(OP(=O)(O)OP(=O)(S)OC[C@H]2O[C@@H](c3csc4c(N)ncnc34)[C@@](O)(C3CC3)[C@@H]2O)C(OC(C)=O)C(OC(C)=O)C1OC(C)=O] |
| Example 268D | 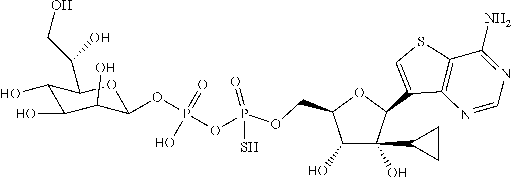 [SMILES: Nc1ncnc2c([C@@H]3O[C@H](COP(=O)(S)OP(=O)(O)OC4OC([C@@H](O)CO)C(O)C(O)C4O)[C@@H](O)[C@]3(O)C3CC3)csc12] |
| Example 269A | 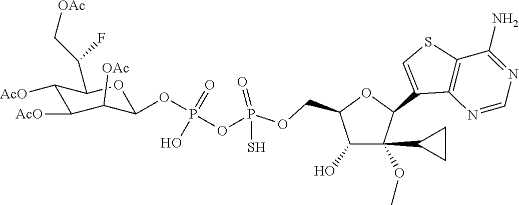 [SMILES: CO[C@]1(C2CC2)[C@H](O)[C@@H](COP(=O)(S)OP(=O)(O)OC2OC([C@@H](F)COC(C)=O)C(OC(C)=O)C(OC(C)=O)C2OC(C)=O)O[C@H]1c1csc2c(N)ncnc12] |
| Example 269B | 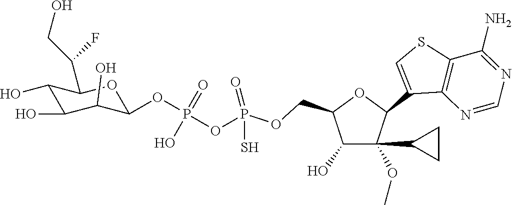 [SMILES: CO[C@]1(C2CC2)[C@H](O)[C@@H](COP(=O)(S)OP(=O)(O)OC2OC([C@@H](F)CO)C(O)C(O)C2O)O[C@H]1c1csc2c(N)ncnc12] |
| Example 269C | 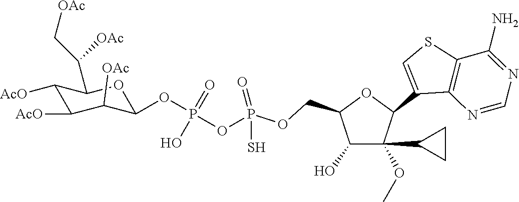 [SMILES: CO[C@]1(C2CC2)[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1csc2c(N)ncnc12] |
| Example 269D | 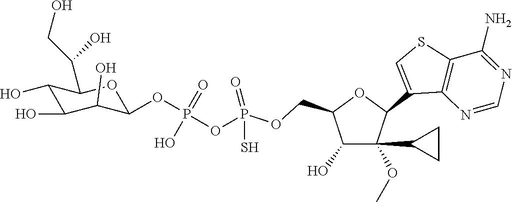 [SMILES: CO[C@]1(C2CC2)[C@H](O)[C@@H](COP(=O)(S)OP(=O)(O)OC2OC([C@@H](O)CO)C(O)C(O)C2O)O[C@H]1c1csc2c(N)ncnc12] |
| Example 270A | 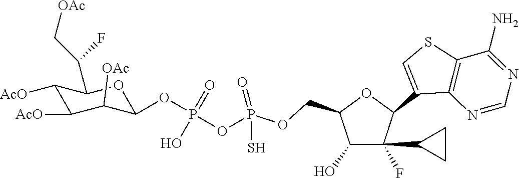 [SMILES: CC(=O)OC[C@H](F)C1OC(OP(=O)(O)OP(=O)(S)OC[C@H]2O[C@@H](c3csc4c(N)ncnc34)[C@@](F)(C3CC3)[C@@H]2O)C(OC(C)=O)C(OC(C)=O)C1OC(C)=O] |
| Example 270B | 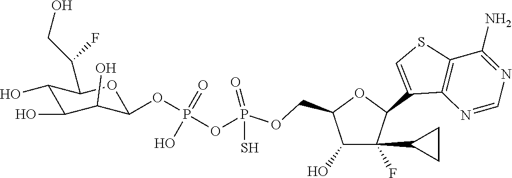 [SMILES: Nc1ncnc2c([C@@H]3O[C@H](COP(=O)(S)OP(=O)(O)OC4OC([C@@H](F)CO)C(O)C(O)C4O)[C@@H](O)[C@]3(F)C3CC3)csc12] |
| Example 270C | 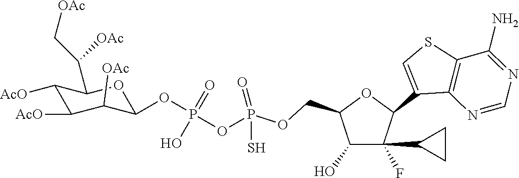 [SMILES: CC(=O)OC[C@H](OC(C)=O)C1OC(OP(=O)(O)OP(=O)(S)OC[C@H]2O[C@@H](c3csc4c(N)ncnc34)[C@@](F)(C3CC3)[C@@H]2O)C(OC(C)=O)C(OC(C)=O)C1OC(C)=O] |
| Example 270D | 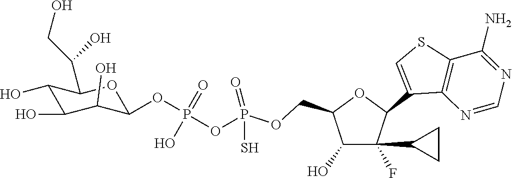 [SMILES: Nc1ncnc2c([C@@H]3O[C@H](COP(=O)(S)OP(=O)(O)OC4OC([C@@H](O)CO)C(O)C(O)C4O)[C@@H](O)[C@]3(F)C3CC3)csc12] |
| Example 271C | 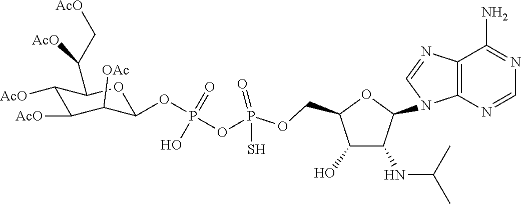 [SMILES: CC(=O)OC[C@H](OC(C)=O)C1OC(OP(=O)(O)OP(=O)(S)OC[C@H]2O[C@@H](n3cnc4c(N)ncnc43)[C@H](NC(C)C)[C@@H]2O)C(OC(C)=O)C(OC(C)=O)C1OC(C)=O] |
| Example 272C | 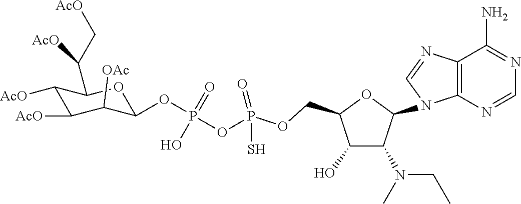 [SMILES: CCN(C)[C@@H]1[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1n1cnc2c(N)ncnc21] |
| Example 274A | 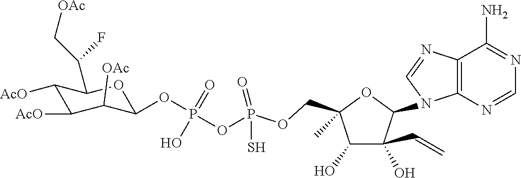 [SMILES: C=C[C@]1(O)[C@H](n2cnc3c(N)ncnc32)O[C@](C)(COP(=O)(S)OP(=O)(O)OC2OC([C@@H](F)COC(C)=O)C(OC(C)=O)C(OC(C)=O)C2OC(C)=O)[C@H]1O] |
| Example 274B | 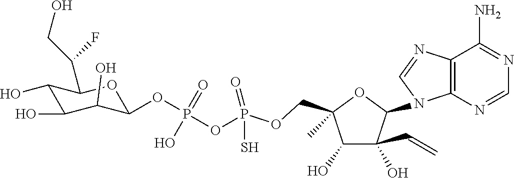 [SMILES: C=C[C@]1(O)[C@H](n2cnc3c(N)ncnc32)O[C@](C)(COP(=O)(S)OP(=O)(O)OC2OC([C@@H](F)CO)C(O)C(O)C2O)[C@H]1O] |
| Example 274C | 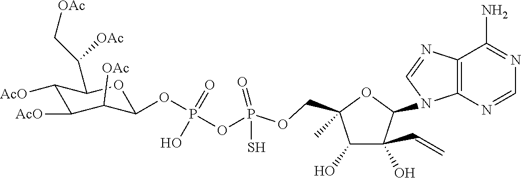 [SMILES: C=C[C@]1(O)[C@H](n2cnc3c(N)ncnc32)O[C@](C)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 274D | 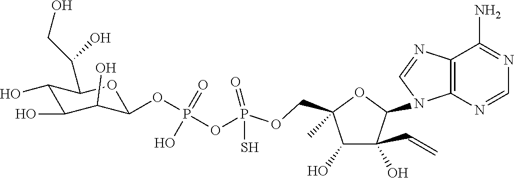 [SMILES: C=C[C@]1(O)[C@H](n2cnc3c(N)ncnc32)O[C@](C)(COP(=O)(S)OP(=O)(O)OC2OC([C@@H](O)CO)C(O)C(O)C2O)[C@H]1O] |
| Example 275A | 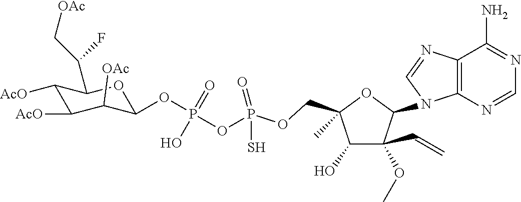 [SMILES: C=C[C@]1(OC)[C@H](n2cnc3c(N)ncnc32)O[C@](C)(COP(=O)(S)OP(=O)(O)OC2OC([C@@H](F)COC(C)=O)C(OC(C)=O)C(OC(C)=O)C2OC(C)=O)[C@H]1O] |
| Example 275B | 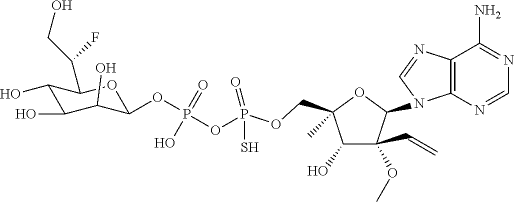 [SMILES: C=C[C@]1(OC)[C@H](n2cnc3c(N)ncnc32)O[C@](C)(COP(=O)(S)OP(=O)(O)OC2OC([C@@H](F)CO)C(O)C(O)C2O)[C@H]1O] |
| Example 275C | 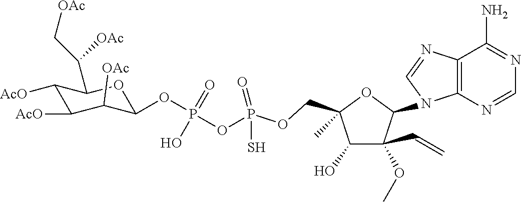 [SMILES: C=C[C@]1(OC)[C@H](n2cnc3c(N)ncnc32)O[C@](C)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 275D | 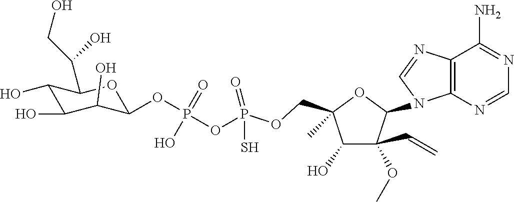 [SMILES: C=C[C@]1(OC)[C@H](n2cnc3c(N)ncnc32)O[C@](C)(COP(=O)(S)OP(=O)(O)OC2OC([C@@H](O)CO)C(O)C(O)C2O)[C@H]1O] |
| Example 276A | 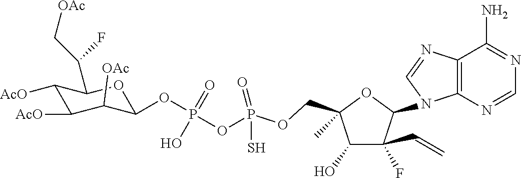 [SMILES: C=C[C@]1(F)[C@H](n2cnc3c(N)ncnc32)O[C@](C)(COP(=O)(S)OP(=O)(O)OC2OC([C@@H](F)COC(C)=O)C(OC(C)=O)C(OC(C)=O)C2OC(C)=O)[C@H]1O] |
| Example 276B | 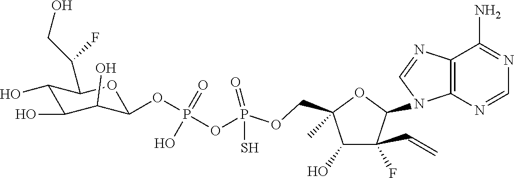 [SMILES: C=C[C@]1(F)[C@H](n2cnc3c(N)ncnc32)O[C@](C)(COP(=O)(S)OP(=O)(O)OC2OC([C@@H](F)CO)C(O)C(O)C2O)[C@H]1O] |
| Example 276C | 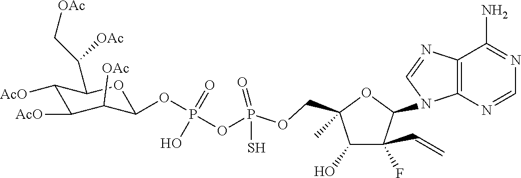 [SMILES: C=C[C@]1(F)[C@H](n2cnc3c(N)ncnc32)O[C@](C)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 276D | 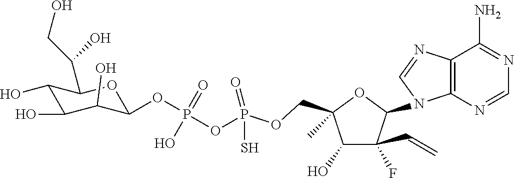 [SMILES: C=C[C@]1(F)[C@H](n2cnc3c(N)ncnc32)O[C@](C)(COP(=O)(S)OP(=O)(O)OC2OC([C@@H](O)CO)C(O)C(O)C2O)[C@H]1O] |
| Example 280A | 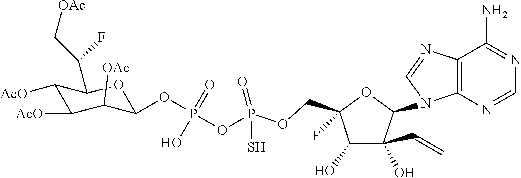 [SMILES: C=C[C@]1(O)[C@H](n2cnc3c(N)ncnc32)O[C@](F)(COP(=O)(S)OP(=O)(O)OC2OC([C@@H](F)COC(C)=O)C(OC(C)=O)C(OC(C)=O)C2OC(C)=O)[C@H]1O] |
| Example 280B | 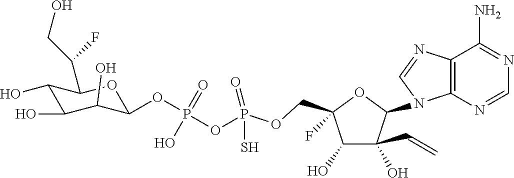 [SMILES: C=C[C@]1(O)[C@H](n2cnc3c(N)ncnc32)O[C@](F)(COP(=O)(S)OP(=O)(O)OC2OC([C@@H](F)CO)C(O)C(O)C2O)[C@H]1O] |
| Example 280C | 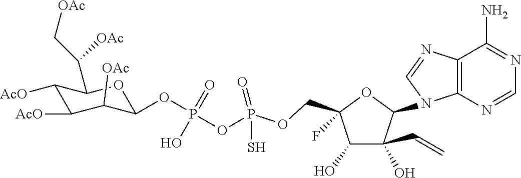 [SMILES: C=C[C@]1(O)[C@H](n2cnc3c(N)ncnc32)O[C@](F)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 280D | 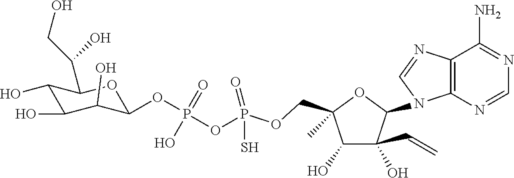 [SMILES: C=C[C@]1(O)[C@H](n2cnc3c(N)ncnc32)O[C@](C)(COP(=O)(S)OP(=O)(O)OC2OC([C@@H](O)CO)C(O)C(O)C2O)[C@H]1O] |
| Example 281A | 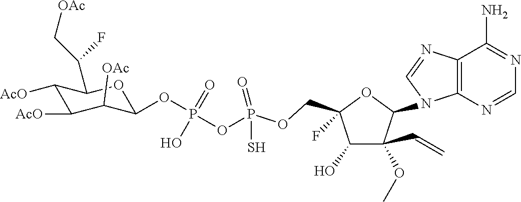 [SMILES: C=C[C@]1(OC)[C@H](n2cnc3c(N)ncnc32)O[C@](F)(COP(=O)(S)OP(=O)(O)OC2OC([C@@H](F)COC(C)=O)C(OC(C)=O)C(OC(C)=O)C2OC(C)=O)[C@H]1O] |
| Example 281B | 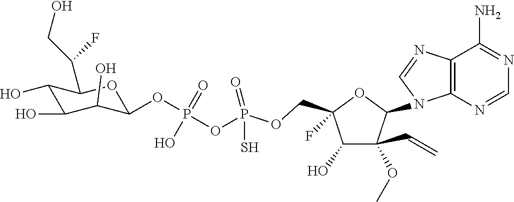 [SMILES: C=C[C@]1(OC)[C@H](n2cnc3c(N)ncnc32)O[C@](F)(COP(=O)(S)OP(=O)(O)OC2OC([C@@H](F)CO)C(O)C(O)C2O)[C@H]1O] |
| Example 281C | 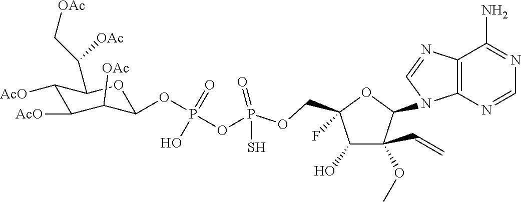 [SMILES: C=C[C@]1(OC)[C@H](n2cnc3c(N)ncnc32)O[C@](F)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 281D | 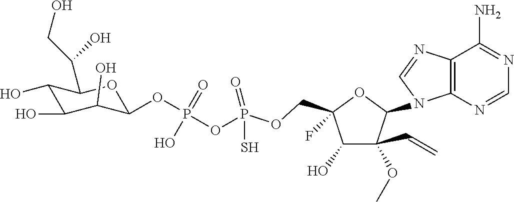 [SMILES: C=C[C@]1(OC)[C@H](n2cnc3c(N)ncnc32)O[C@](F)(COP(=O)(S)OP(=O)(O)OC2OC([C@@H](O)CO)C(O)C(O)C2O)[C@H]1O] |
| Example 282A | 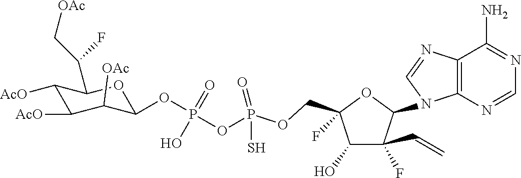 [SMILES: C=C[C@]1(F)[C@H](n2cnc3c(N)ncnc32)O[C@](F)(COP(=O)(S)OP(=O)(O)OC2OC([C@@H](F)COC(C)=O)C(OC(C)=O)C(OC(C)=O)C2OC(C)=O)[C@H]1O] |
| Example 282B | 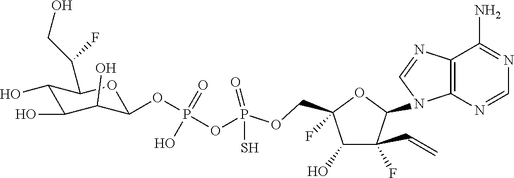 [SMILES: C=C[C@]1(F)[C@H](n2cnc3c(N)ncnc32)O[C@](F)(COP(=O)(S)OP(=O)(O)OC2OC([C@@H](F)CO)C(O)C(O)C2O)[C@H]1O] |
| Example 282C | 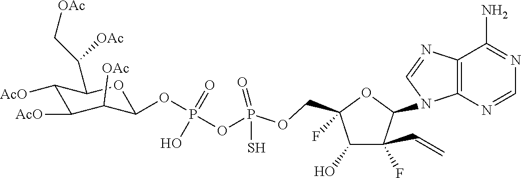 [SMILES: C=C[C@]1(F)[C@H](n2cnc3c(N)ncnc32)O[C@](F)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 282D | 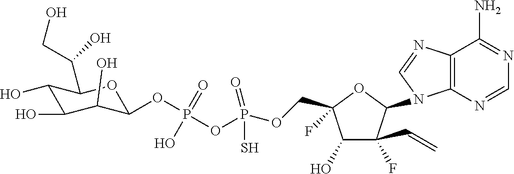 [SMILES: C=C[C@]1(F)[C@H](n2cnc3c(N)ncnc32)O[C@](F)(COP(=O)(S)OP(=O)(O)OC2OC([C@@H](O)CO)C(O)C(O)C2O)[C@H]1O] |
| Example 286A | 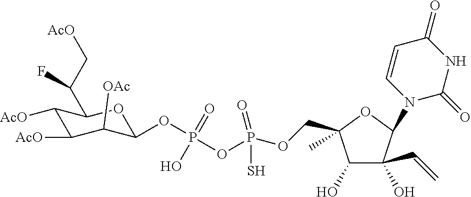 [SMILES: C=C[C@]1(O)[C@H](n2ccc(=O)[nH]c2=O)O[C@](C)(COP(=O)(S)OP(=O)(O)OC2OC([C@@H](F)COC(C)=O)C(OC(C)=O)C(OC(C)=O)C2OC(C)=O)[C@H]1O] |
| Example 286B | 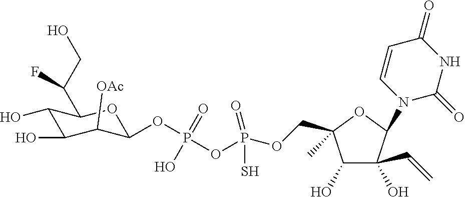 [SMILES: C=C[C@]1(O)[C@H](n2ccc(=O)[nH]c2=O)O[C@](C)(COP(=O)(S)OP(=O)(O)OC2OC([C@@H](F)CO)C(O)C(O)C2OC(C)=O)[C@H]1O] |
| Example 286C | 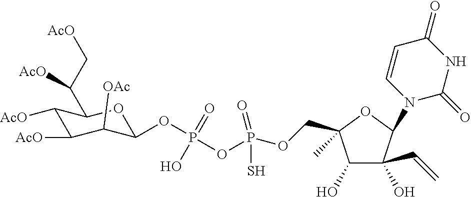 [SMILES: C=C[C@]1(O)[C@H](n2ccc(=O)[nH]c2=O)O[C@](C)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 286D | 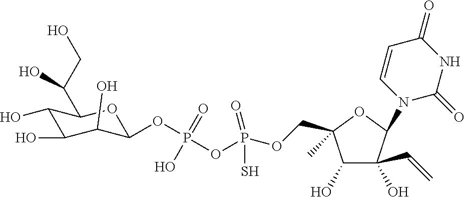 [SMILES: C=C[C@]1(O)[C@H](n2ccc(=O)[nH]c2=O)O[C@](C)(COP(=O)(S)OP(=O)(O)OC2OC([C@@H](O)CO)C(O)C(O)C2O)[C@H]1O] |
| Example 287A | 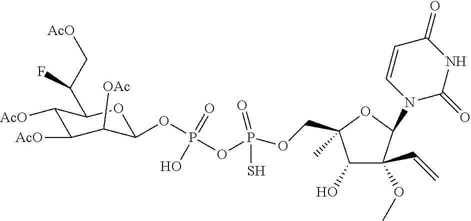 [SMILES: C=C[C@]1(OC)[C@H](n2ccc(=O)[nH]c2=O)O[C@](C)(COP(=O)(S)OP(=O)(O)OC2OC([C@@H](F)COC(C)=O)C(OC(C)=O)C(OC(C)=O)C2OC(C)=O)[C@H]1O] |
| Example 287B | 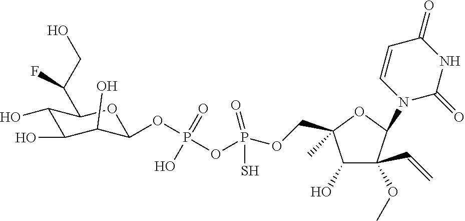 [SMILES: C=C[C@]1(OC)[C@H](n2ccc(=O)[nH]c2=O)O[C@](C)(COP(=O)(S)OP(=O)(O)OC2OC([C@@H](F)CO)C(O)C(O)C2O)[C@H]1O] |
| Example 287C | 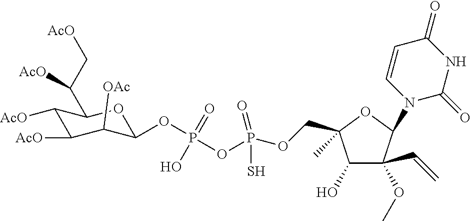 [SMILES: C=C[C@]1(OC)[C@H](n2ccc(=O)[nH]c2=O)O[C@](C)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 287D | 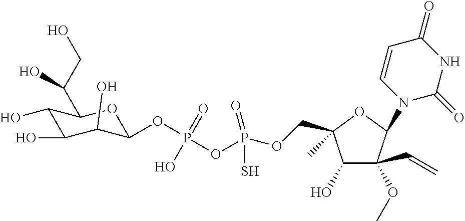 [SMILES: C=C[C@]1(OC)[C@H](n2ccc(=O)[nH]c2=O)O[C@](C)(COP(=O)(S)OP(=O)(O)OC2OC([C@@H](O)CO)C(O)C(O)C2O)[C@H]1O] |
| Example 288A | 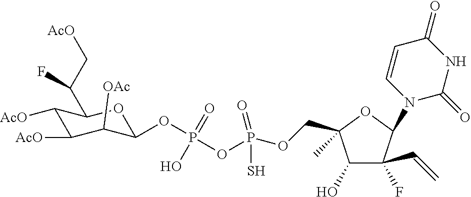 [SMILES: C=C[C@]1(F)[C@H](n2ccc(=O)[nH]c2=O)O[C@](C)(COP(=O)(S)OP(=O)(O)OC2OC([C@@H](F)COC(C)=O)C(OC(C)=O)C(OC(C)=O)C2OC(C)=O)[C@H]1O] |
| Example 288B | 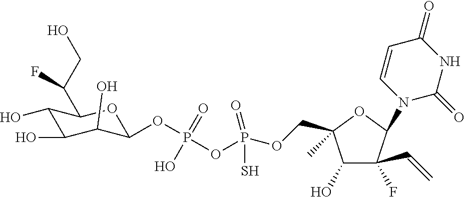 [SMILES: C=C[C@]1(F)[C@H](n2ccc(=O)[nH]c2=O)O[C@](C)(COP(=O)(S)OP(=O)(O)OC2OC([C@@H](F)CO)C(O)C(O)C2O)[C@H]1O] |
| Example 288C | 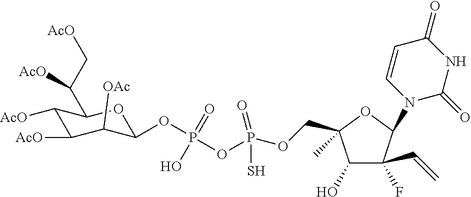 [SMILES: C=C[C@]1(F)[C@H](n2ccc(=O)[nH]c2=O)O[C@](C)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 288D | 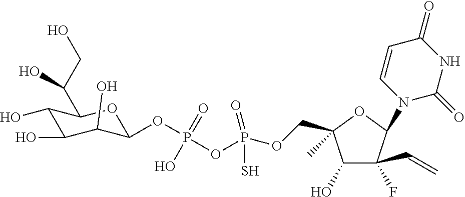 [SMILES: C=C[C@]1(F)[C@H](n2ccc(=O)[nH]c2=O)O[C@](C)(COP(=O)(S)OP(=O)(O)OC2OC([C@@H](O)CO)C(O)C(O)C2O)[C@H]1O] |
| Example 292A | 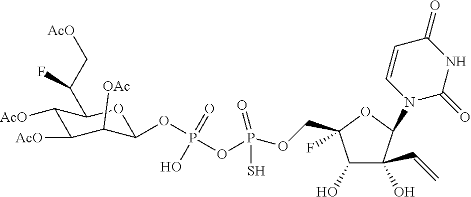 [SMILES: C=C[C@]1(O)[C@H](n2ccc(=O)[nH]c2=O)O[C@](F)(COP(=O)(S)OP(=O)(O)OC2OC([C@@H](F)COC(C)=O)C(OC(C)=O)C(OC(C)=O)C2OC(C)=O)[C@H]1O] |
| Example 292B | 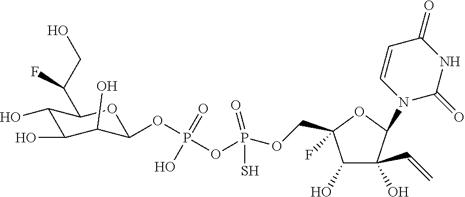 [SMILES: C=C[C@]1(O)[C@H](n2ccc(=O)[nH]c2=O)O[C@](F)(COP(=O)(S)OP(=O)(O)OC2OC([C@@H](F)CO)C(O)C(O)C2O)[C@H]1O] |
| Example 292C | 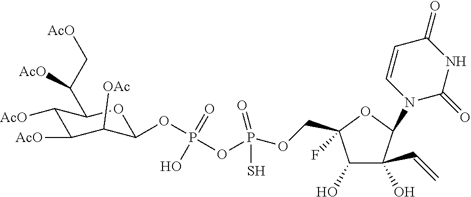 [SMILES: C=C[C@]1(O)[C@H](n2ccc(=O)[nH]c2=O)O[C@](F)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 292D | 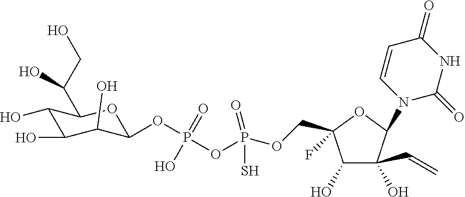 [SMILES: C=C[C@]1(O)[C@H](n2ccc(=O)[nH]c2=O)O[C@](F)(COP(=O)(S)OP(=O)(O)OC2OC([C@@H](O)CO)C(O)C(O)C2O)[C@H]1O] |
| Example 293A | 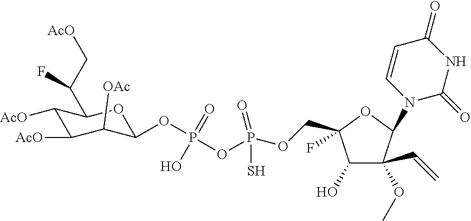 [SMILES: C=C[C@]1(OC)[C@H](n2ccc(=O)[nH]c2=O)O[C@](F)(COP(=O)(S)OP(=O)(O)OC2OC([C@@H](F)COC(C)=O)C(OC(C)=O)C(OC(C)=O)C2OC(C)=O)[C@H]1O] |
| Example 293B | 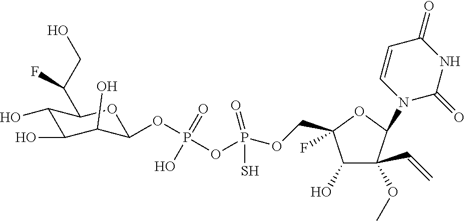 [SMILES: C=C[C@]1(OC)[C@H](n2ccc(=O)[nH]c2=O)O[C@](F)(COP(=O)(S)OP(=O)(O)OC2OC([C@@H](F)CO)C(O)C(O)C2O)[C@H]1O] |
| Example 293C | 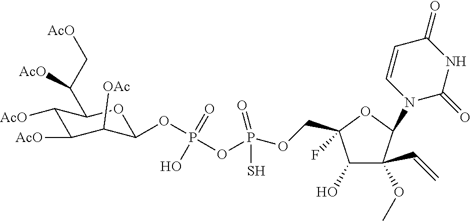 [SMILES: C=C[C@]1(OC)[C@H](n2ccc(=O)[nH]c2=O)O[C@](F)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 293D | 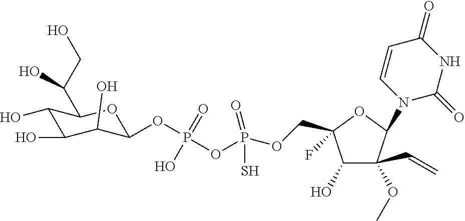 [SMILES: C=C[C@]1(OC)[C@H](n2ccc(=O)[nH]c2=O)O[C@](F)(COP(=O)(S)OP(=O)(O)OC2OC([C@@H](O)CO)C(O)C(O)C2O)[C@H]1O] |
| Example 294A | 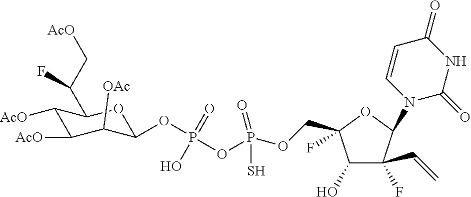 [SMILES: C=C[C@]1(F)[C@H](n2ccc(=O)[nH]c2=O)O[C@](F)(COP(=O)(S)OP(=O)(O)OC2OC([C@@H](F)COC(C)=O)C(OC(C)=O)C(OC(C)=O)C2OC(C)=O)[C@H]1O] |
| Example 294B | 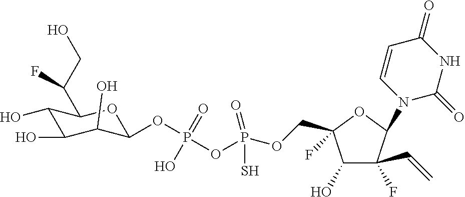 [SMILES: C=C[C@]1(F)[C@H](n2ccc(=O)[nH]c2=O)O[C@](F)(COP(=O)(S)OP(=O)(O)OC2OC([C@@H](F)CO)C(O)C(O)C2O)[C@H]1O] |
| Example 294C | 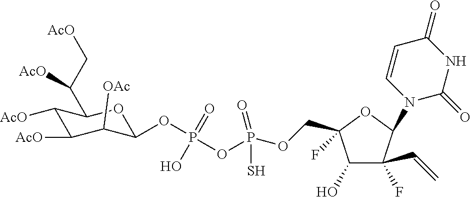 [SMILES: C=C[C@]1(F)[C@H](n2ccc(=O)[nH]c2=O)O[C@](F)(COP(=O)(S)OP(=O)(O)OC2OC([C@H](COC(C)=O)OC(C)=O)C(OC(C)=O)C(OC(C)=O)C2OC(C)=O)[C@H]1O] |
| Example 294D | 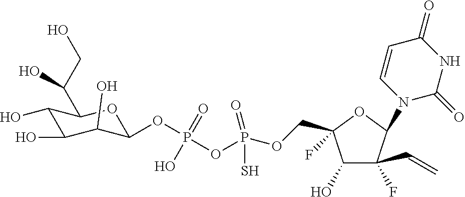 [SMILES: C=C[C@]1(F)[C@H](n2ccc(=O)[nH]c2=O)O[C@](F)(COP(=O)(S)OP(=O)(O)OC2OC([C@@H](O)CO)C(O)C(O)C2O)[C@H]1O] |
| Example 298A | 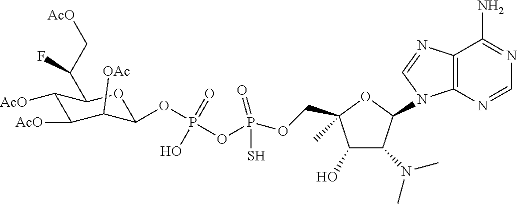 [SMILES: CC(=O)OC[C@H](F)C1OC(OP(=O)(O)OP(=O)(S)OC[C@@]2(C)O[C@@H](n3cnc4c(N)ncnc43)[C@H](N(C)C)[C@@H]2O)C(OC(C)=O)C(OC(C)=O)C1OC(C)=O] |
| Example 298B | 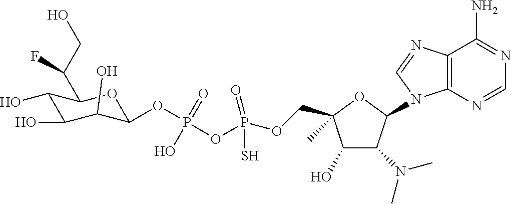 [SMILES: CN(C)[C@H]1[C@H](n2cnc3c(N)ncnc32)O[C@](C)(COP(=O)(S)OP(=O)(O)OC2OC([C@@H](F)CO)C(O)C(O)C2O)[C@H]1O] |
| Example 298C | 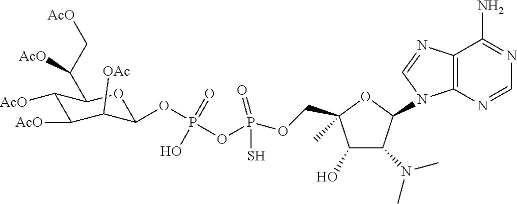 [SMILES: CC(=O)OC[C@H](OC(C)=O)C1OC(OP(=O)(O)OP(=O)(S)OC[C@@]2(C)O[C@@H](n3cnc4c(N)ncnc43)[C@H](N(C)C)[C@@H]2O)C(OC(C)=O)C(OC(C)=O)C1OC(C)=O] |
| Example 298D | 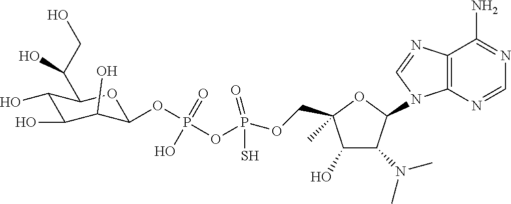 [SMILES: CN(C)[C@H]1[C@H](n2cnc3c(N)ncnc32)O[C@](C)(COP(=O)(S)OP(=O)(O)OC2OC([C@@H](O)CO)C(O)C(O)C2O)[C@H]1O] |
| Example 299A | 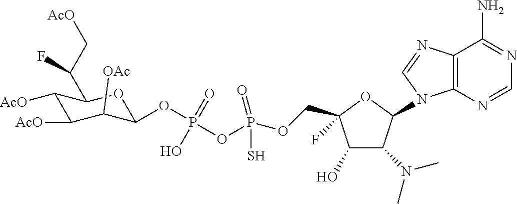 [SMILES: CC(=O)OC[C@H](F)C1OC(OP(=O)(O)OP(=O)(S)OC[C@@]2(F)O[C@@H](n3cnc4c(N)ncnc43)[C@H](N(C)C)[C@@H]2O)C(OC(C)=O)C(OC(C)=O)C1OC(C)=O] |
| Example 299B | 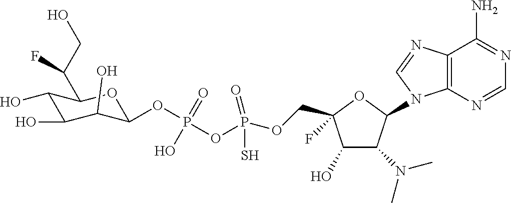 [SMILES: CN(C)[C@H]1[C@H](n2cnc3c(N)ncnc32)O[C@](F)(COP(=O)(S)OP(=O)(O)OC2OC([C@@H](F)CO)C(O)C(O)C2O)[C@H]1O] |
| Example 299C | 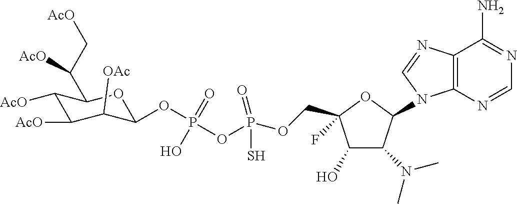 [SMILES: CC(=O)OC[C@H](OC(C)=O)C1OC(OP(=O)(O)OP(=O)(S)OC[C@@]2(F)O[C@@H](n3cnc4c(N)ncnc43)[C@H](N(C)C)[C@@H]2O)C(OC(C)=O)C(OC(C)=O)C1OC(C)=O] |
| Example 299D | 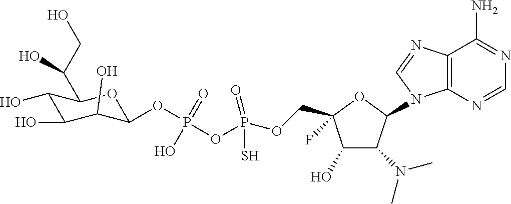 [SMILES: CN(C)[C@H]1[C@H](n2cnc3c(N)ncnc32)O[C@](F)(COP(=O)(S)OP(=O)(O)OC2OC([C@@H](O)CO)C(O)C(O)C2O)[C@H]1O] |
| Example 300A | 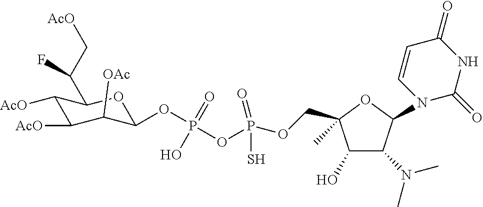 [SMILES: CC(=O)OC[C@H](F)C1OC(OP(=O)(O)OP(=O)(S)OC[C@@]2(C)O[C@@H](n3ccc(=O)[nH]c3=O)[C@H](N(C)C)[C@@H]2O)C(OC(C)=O)C(OC(C)=O)C1OC(C)=O] |
| Example 300B | 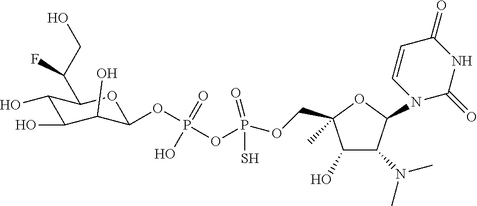 [SMILES: CN(C)[C@H]1[C@H](n2ccc(=O)[nH]c2=O)O[C@](C)(COP(=O)(S)OP(=O)(O)OC2OC([C@@H](F)CO)C(O)C(O)C2O)[C@H]1O] |
| Example 300C | 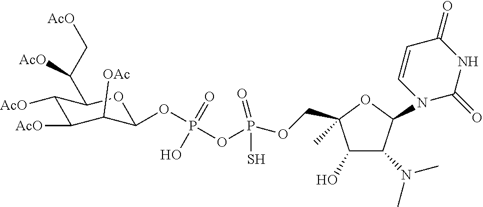 [SMILES: CC(=O)OC[C@H](OC(C)=O)C1OC(OP(=O)(O)OP(=O)(S)OC[C@@]2(C)O[C@@H](n3ccc(=O)[nH]c3=O)[C@H](N(C)C)[C@@H]2O)C(OC(C)=O)C(OC(C)=O)C1OC(C)=O] |
| Example 300D | 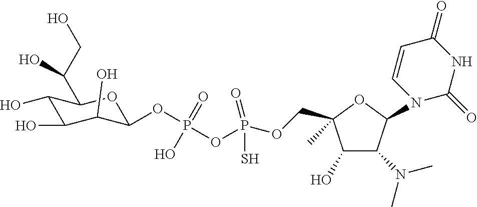 [SMILES: CN(C)[C@H]1[C@H](n2ccc(=O)[nH]c2=O)O[C@](C)(COP(=O)(S)OP(=O)(O)OC2OC([C@@H](O)CO)C(O)C(O)C2O)[C@H]1O] |
| Example 301A | 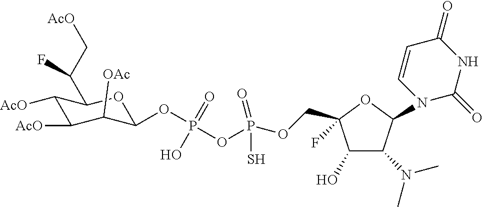 [SMILES: CC(=O)OC[C@H](F)C1OC(OP(=O)(O)OP(=O)(S)OC[C@@]2(F)O[C@@H](n3ccc(=O)[nH]c3=O)[C@H](N(C)C)[C@@H]2O)C(OC(C)=O)C(OC(C)=O)C1OC(C)=O] |
| Example 301B | 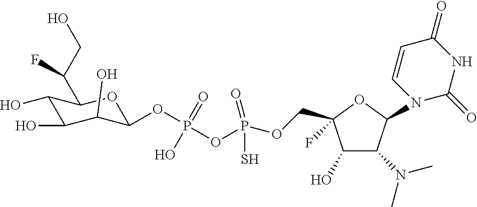 [SMILES: CN(C)[C@H]1[C@H](n2ccc(=O)[nH]c2=O)O[C@](F)(COP(=O)(S)OP(=O)(O)OC2OC([C@@H](F)CO)C(O)C(O)C2O)[C@H]1O] |
| Example 301C | 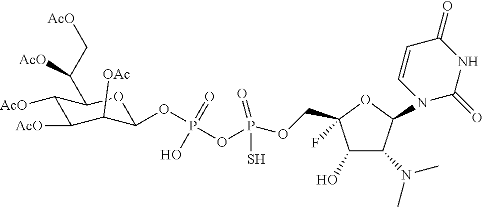 [SMILES: CC(=O)OC[C@H](OC(C)=O)C1OC(OP(=O)(O)OP(=O)(S)OC[C@@]2(F)O[C@@H](n3ccc(=O)[nH]c3=O)[C@H](N(C)C)[C@@H]2O)C(OC(C)=O)C(OC(C)=O)C1OC(C)=O] |
| Example 301D | 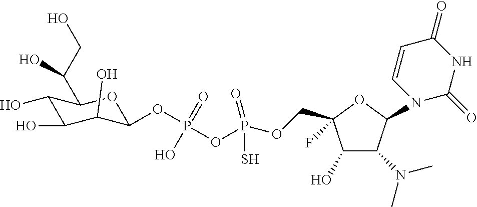 [SMILES: CN(C)[C@H]1[C@H](n2ccc(=O)[nH]c2=O)O[C@](F)(COP(=O)(S)OP(=O)(O)OC2OC([C@@H](O)CO)C(O)C(O)C2O)[C@H]1O] |
| Example 310A | 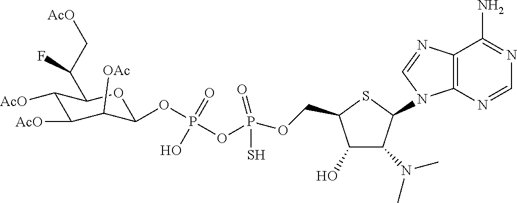 [SMILES: CC(=O)OC[C@H](F)C1OC(OP(=O)(O)OP(=O)(S)OC[C@H]2S[C@@H](n3cnc4c(N)ncnc43)[C@H](N(C)C)[C@@H]2O)C(OC(C)=O)C(OC(C)=O)C1OC(C)=O] |
| Example 310B | 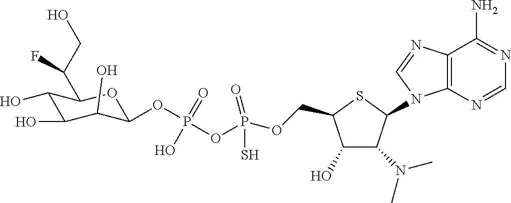 [SMILES: CN(C)[C@@H]1[C@H](O)[C@@H](COP(=O)(S)OP(=O)(O)OC2OC([C@@H](F)CO)C(O)C(O)C2O)S[C@H]1n1cnc2c(N)ncnc21] |
| Example 310C | 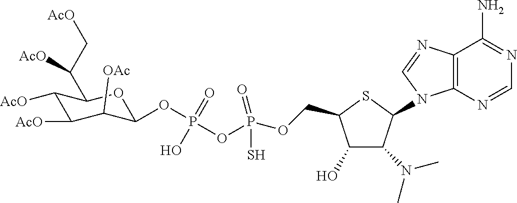 [SMILES: CC(=O)OC[C@H](OC(C)=O)C1OC(OP(=O)(O)OP(=O)(S)OC[C@H]2S[C@@H](n3cnc4c(N)ncnc43)[C@H](N(C)C)[C@@H]2O)C(OC(C)=O)C(OC(C)=O)C1OC(C)=O] |
| Example 310D | 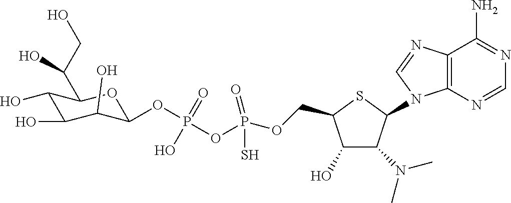 [SMILES: CN(C)[C@@H]1[C@H](O)[C@@H](COP(=O)(S)OP(=O)(O)OC2OC([C@@H](O)CO)C(O)C(O)C2O)S[C@H]1n1cnc2c(N)ncnc21] |
| Example 311A | 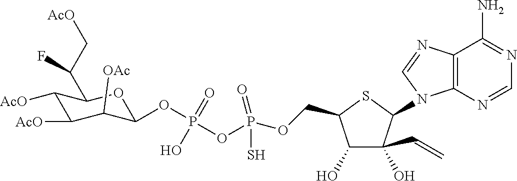 [SMILES: C=C[C@@]1(O)[C@H](O)[C@@H](COP(=O)(S)OP(=O)(O)OC2OC([C@@H](F)COC(C)=O)C(OC(C)=O)C(OC(C)=O)C2OC(C)=O)S[C@H]1n1cnc2c(N)ncnc21] |
| Example 311B | 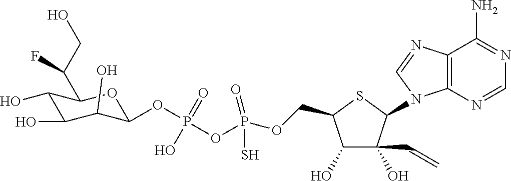 [SMILES: C=C[C@@]1(O)[C@H](O)[C@@H](COP(=O)(S)OP(=O)(O)OC2OC([C@@H](F)CO)C(O)C(O)C2O)S[C@H]1n1cnc2c(N)ncnc21] |
| Example 311C | 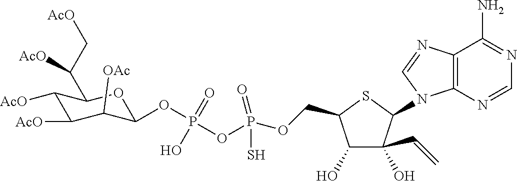 [SMILES: C=C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)S[C@H]1n1cnc2c(N)ncnc21] |
| Example 311D | 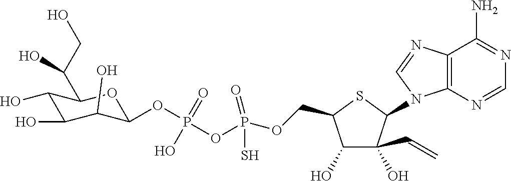 [SMILES: C=C[C@@]1(O)[C@H](O)[C@@H](COP(=O)(S)OP(=O)(O)OC2OC([C@@H](O)CO)C(O)C(O)C2O)S[C@H]1n1cnc2c(N)ncnc21] |
| Example 312A | 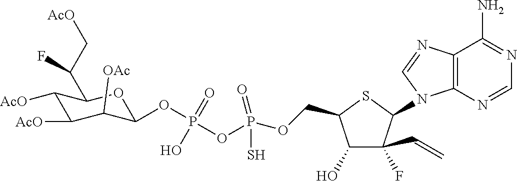 [SMILES: C=C[C@@]1(F)[C@H](O)[C@@H](COP(=O)(S)OP(=O)(O)OC2OC([C@@H](F)COC(C)=O)C(OC(C)=O)C(OC(C)=O)C2OC(C)=O)S[C@H]1n1cnc2c(N)ncnc21] |
| Example 312B | 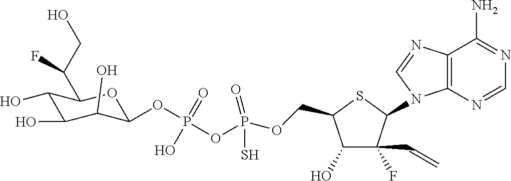 [SMILES: C=C[C@@]1(F)[C@H](O)[C@@H](COP(=O)(S)OP(=O)(O)OC2OC([C@@H](F)CO)C(O)C(O)C2O)S[C@H]1n1cnc2c(N)ncnc21] |
| Example 312C | 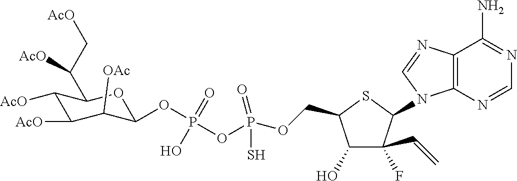 [SMILES: C=C[C@@]1(F)[C@H](O)[C@@H](COP(=O)(S)OP(=O)(O)OC2OC([C@H](COC(C)=O)OC(C)=O)C(OC(C)=O)C(OC(C)=O)C2OC(C)=O)S[C@H]1n1cnc2c(N)ncnc21] |
| Example 312D | 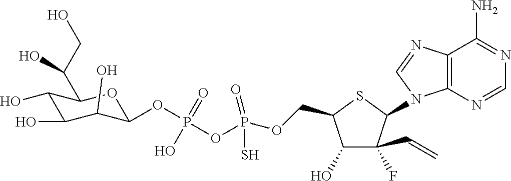 [SMILES: C=C[C@@]1(F)[C@H](O)[C@@H](COP(=O)(S)OP(=O)(O)OC2OC([C@@H](O)CO)C(O)C(O)C2O)S[C@H]1n1cnc2c(N)ncnc21] |
| Example 313A | 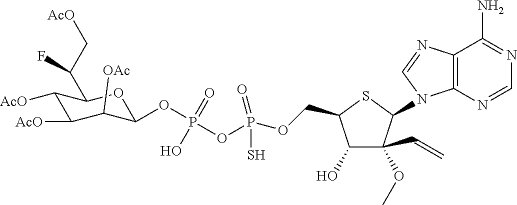 [SMILES: C=C[C@@]1(OC)[C@H](O)[C@@H](COP(=O)(S)OP(=O)(O)OC2OC([C@@H](F)COC(C)=O)C(OC(C)=O)C(OC(C)=O)C2OC(C)=O)S[C@H]1n1cnc2c(N)ncnc21] |
| Example 313B | 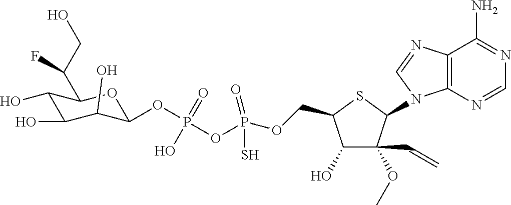 [SMILES: C=C[C@@]1(OC)[C@H](O)[C@@H](COP(=O)(S)OP(=O)(O)OC2OC([C@@H](F)CO)C(O)C(O)C2O)S[C@H]1n1cnc2c(N)ncnc21] |
| Example 313C | 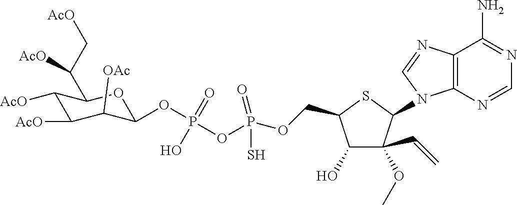 [SMILES: C=C[C@@]1(OC)[C@H](O)[C@@H](COP(=O)(S)OP(=O)(O)OC2OC([C@H](COC(C)=O)OC(C)=O)C(OC(C)=O)C(OC(C)=O)C2OC(C)=O)S[C@H]1n1cnc2c(N)ncnc21] |
| Example 313D | 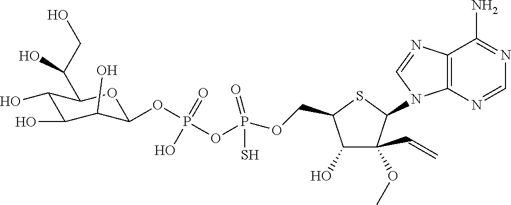 [SMILES: C=C[C@@]1(OC)[C@H](O)[C@@H](COP(=O)(S)OP(=O)(O)OC2OC([C@@H](O)CO)C(O)C(O)C2O)S[C@H]1n1cnc2c(N)ncnc21] |
| Example 316A | 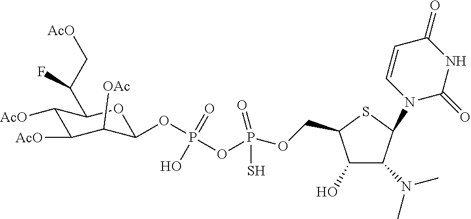 [SMILES: CC(=O)OC[C@H](F)C1OC(OP(=O)(O)OP(=O)(S)OC[C@H]2S[C@@H](n3ccc(=O)[nH]c3=O)[C@H](N(C)C)[C@@H]2O)C(OC(C)=O)C(OC(C)=O)C1OC(C)=O] |
| Example 316B | 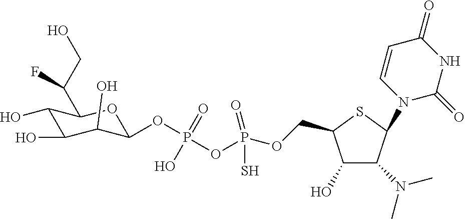 [SMILES: CN(C)[C@@H]1[C@H](O)[C@@H](COP(=O)(S)OP(=O)(O)OC2OC([C@@H](F)CO)C(O)C(O)C2O)S[C@H]1n1ccc(=O)[nH]c1=O] |
| Example 316C | 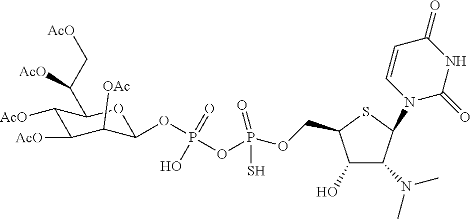 [SMILES: CC(=O)OC[C@H](OC(C)=O)C1OC(OP(=O)(O)OP(=O)(S)OC[C@H]2S[C@@H](n3ccc(=O)[nH]c3=O)[C@H](N(C)C)[C@@H]2O)C(OC(C)=O)C(OC(C)=O)C1OC(C)=O] |
| Example 316D | 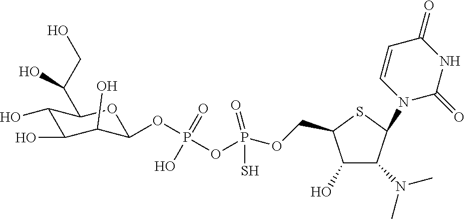 [SMILES: CN(C)[C@@H]1[C@H](O)[C@@H](COP(=O)(S)OP(=O)(O)OC2OC([C@@H](O)CO)C(O)C(O)C2O)S[C@H]1n1ccc(=O)[nH]c1=O] |
| Example 317A | 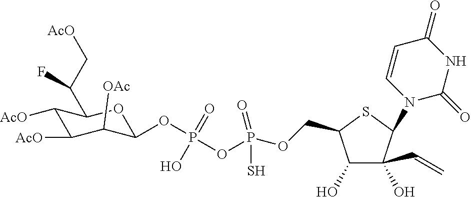 [SMILES: C=C[C@@]1(O)[C@H](O)[C@@H](COP(=O)(S)OP(=O)(O)OC2OC([C@@H](F)COC(C)=O)C(OC(C)=O)C(OC(C)=O)C2OC(C)=O)S[C@H]1n1ccc(=O)[nH]c1=O] |
| Example 317B | 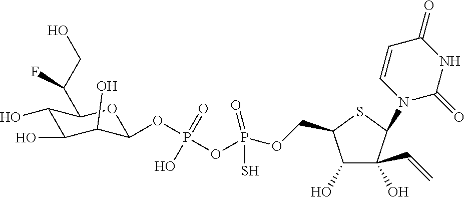 [SMILES: C=C[C@@]1(O)[C@H](O)[C@@H](COP(=O)(S)OP(=O)(O)OC2OC([C@@H](F)CO)C(O)C(O)C2O)S[C@H]1n1ccc(=O)[nH]c1=O] |
| Example 317C | 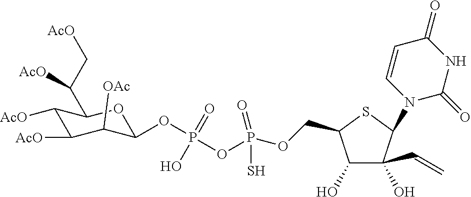 [SMILES: C=C[C@@]1(O)[C@H](O)[C@@H](COP(=O)(S)OP(=O)(O)OC2OC([C@H](COC(C)=O)OC(C)=O)C(OC(C)=O)C(OC(C)=O)C2OC(C)=O)S[C@H]1n1ccc(=O)[nH]c1=O] |
| Example 317D | 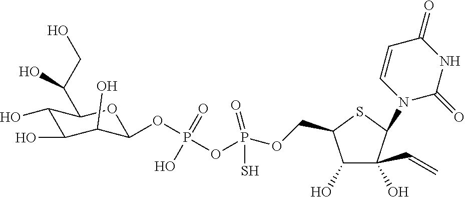 [SMILES: C=C[C@@]1(O)[C@H](O)[C@@H](COP(=O)(S)OP(=O)(O)OC2OC([C@@H](O)CO)C(O)C(O)C2O)S[C@H]1n1ccc(=O)[nH]c1=O] |
| Example 318A | 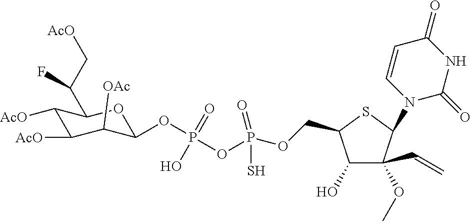 [SMILES: C=C[C@@]1(OC)[C@H](O)[C@@H](COP(=O)(S)OP(=O)(O)OC2OC([C@@H](F)COC(C)=O)C(OC(C)=O)C(OC(C)=O)C2OC(C)=O)S[C@H]1n1ccc(=O)[nH]c1=O] |
| Example 318B | 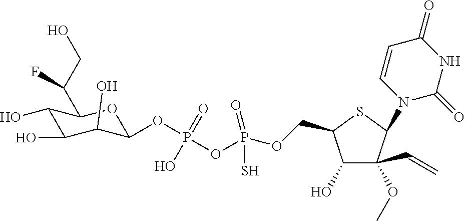 [SMILES: C=C[C@@]1(OC)[C@H](O)[C@@H](COP(=O)(S)OP(=O)(O)OC2OC([C@@H](F)CO)C(O)C(O)C2O)S[C@H]1n1ccc(=O)[nH]c1=O] |
| Example 318C | 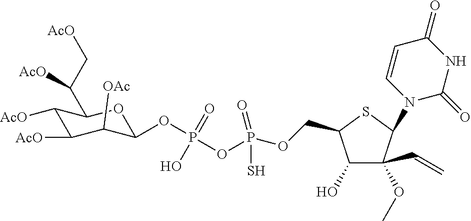 [SMILES: C=C[C@@]1(OC)[C@H](O)[C@@H](COP(=O)(S)OP(=O)(O)OC2OC([C@H](COC(C)=O)OC(C)=O)C(OC(C)=O)C(OC(C)=O)C2OC(C)=O)S[C@H]1n1ccc(=O)[nH]c1=O] |
| Example 318D | 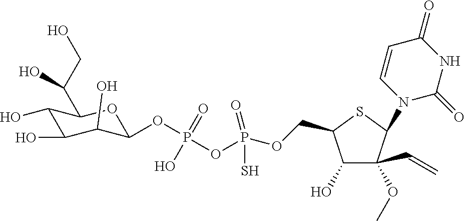 [SMILES: C=C[C@@]1(OC)[C@H](O)[C@@H](COP(=O)(S)OP(=O)(O)OC2OC([C@@H](O)CO)C(O)C(O)C2O)S[C@H]1n1ccc(=O)[nH]c1=O] |
| Example 319A | 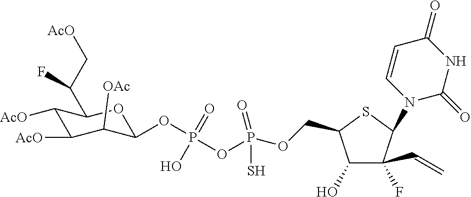 [SMILES: C=C[C@@]1(F)[C@H](O)[C@@H](COP(=O)(S)OP(=O)(O)OC2OC([C@@H](F)COC(C)=O)C(OC(C)=O)C(OC(C)=O)C2OC(C)=O)S[C@H]1n1ccc(=O)[nH]c1=O] |
| Example 319B | 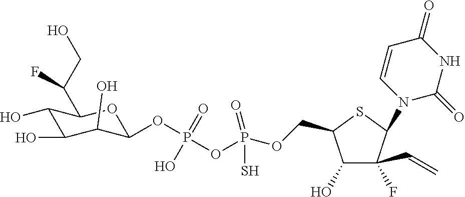 [SMILES: C=C[C@@]1(F)[C@H](O)[C@@H](COP(=O)(S)OP(=O)(O)OC2OC([C@@H](F)CO)C(O)C(O)C2O)S[C@H]1n1ccc(=O)[nH]c1=O] |
| Example 319C | 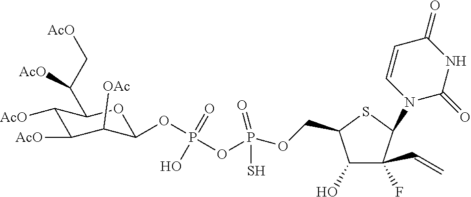 [SMILES: C=C[C@@]1(F)[C@H](O)[C@@H](COP(=O)(S)OP(=O)(O)OC2OC([C@H](COC(C)=O)OC(C)=O)C(OC(C)=O)C(OC(C)=O)C2OC(C)=O)S[C@H]1n1ccc(=O)[nH]c1=O] |
| Example 319D | 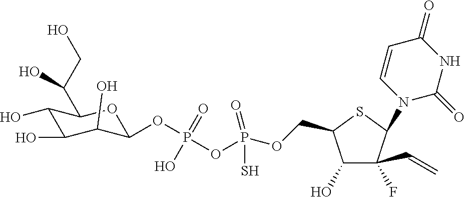 [SMILES: C=C[C@@]1(F)[C@H](O)[C@@H](COP(=O)(S)OP(=O)(O)OC2OC([C@@H](O)CO)C(O)C(O)C2O)S[C@H]1n1ccc(=O)[nH]c1=O] |
| Example 321C | 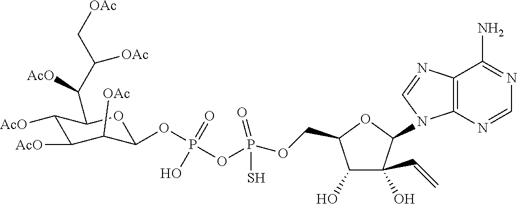 [SMILES: C=C[C@@]1(O)[C@H](O)[C@@H](COP(=O)(S)OP(=O)(O)OC2OC([C@@H](OC(C)=O)C(COC(C)=O)OC(C)=O)C(OC(C)=O)C(OC(C)=O)C2OC(C)=O)O[C@H]1n1cnc2c(N)ncnc21] |
| Example 322C | 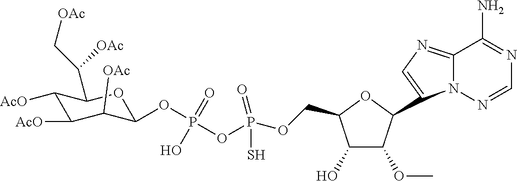 [SMILES: CO[C@@H]1[C@H](O)[C@@H](COP(=O)(S)OP(=O)(O)OC2OC([C@H](COC(C)=O)OC(C)=O)C(OC(C)=O)C(OC(C)=O)C2OC(C)=O)O[C@H]1c1cnc2c(N)ncnn12] |

TABLE 2
Enzymic and cell potency for representative
compounds described herein

| | Activity on | Activity on |
|---|---|---|
| | Enzymatic | Cellular |
| | Assay | Assay |
| Examples | (EC₅₀, nM) | (EC₅₀, nM) |
| Example 21C | | 15.17 |
| Example 21D | 26 | 4.08 |
| Example 22C | | 49.14 |
| Example 22D | 64 | 5.66 |
| Example 25A | | 244 |
| Example 25C | | 20 |
| Example 25D | 17 | 8.41 |
| Example 26C | | 45.50 |
| Example 26E | | >3000 |
| Example 31C | | 16.93 |
| Example 31D | 24 | 16.04 |
| Example 31E | | 68.28 |
| Example 31H | | 0.15 |
| Example 31K | | 57.61 |
| Example 31L | | 13.83 |
| Example 31M | | 2.99 |
| Example 31O | | 21.00 |
| Example 31P | | 7.23 |
| Example 31Q | 11 | 7.01 |
| Example 31R | 12 | 13.63 |
| Example 33C | | 40.15 |
| Example 33E | | 433.5 |
| Example 34C | | 344.9 |
| Example 34D | | 166.7 |
| Example 35C | | 18.93 |
| Example 35D | | 38.67 |
| Example 35E | | 197.9 |
| Example 35G | | 16.91 |
| Example 35H | | 16.72 |
| Example 35I | | 10.87 |
| Example 35J | | 10.33 |
| Example 35K | | 34.24 |
| Example 36C | | 1624 |
| Example 37C | | >3000 |
| Example 37D | | >3000 |
| Example 40C | | 431.2 |
| Example 40D | | 713 |
| Example 41C | | 8.31 |
| Example 41D | 4.1 | 5.71 |
| Example 42C | | 7.57 |
| Example 42D | 11 | 13.97 |
| Example 43C | | 20.68 |
| Example 43D | 48 | 18.26 |
| Example 45C | | 9.58 |
| Example 45E | | 7.77 |
| Example 95C | | 34.83 |
| Example 97C | | 1061 |
| Example 97D | | >1000 |
| Example 98C | | 2307 |
| Example 108C | | >3000 |
| Example 110C | | >3000 |
| Example 112C | | 360.4 |
| Example 125C | | 2.89 |
| Example 126C | | 230.9 |
| Example 127C | | 2.94 |
| Example 128C | | 3.62 |
| Example 262C | | 36.27 |
| Example 262D | | 101.2 |
| Example 264C | | 48.28 |
| Example 265C | | 45.35 |
| Example 321C | | >1000 |
| Example 322C | | 6.76 |

A number of embodiments of the invention have been described. Nevertheless, it will be understood that various modifications may be made without departing from the spirit and scope of the invention. Accordingly, other embodiments are within the scope of the following claims.

1. A compound of Formula (X): 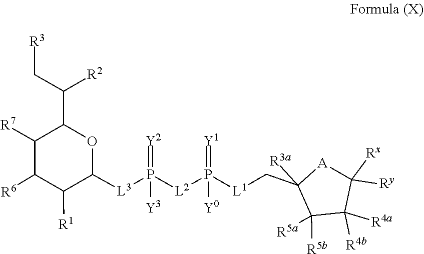
or a pharmaceutically acceptable salt, a stereoisomer, stable isotope, prodrug, or a tautomer thereof, wherein:
R^{X} is:
(A) a moiety having formula (X-Ia), (X-Ib), or (X-Ic): 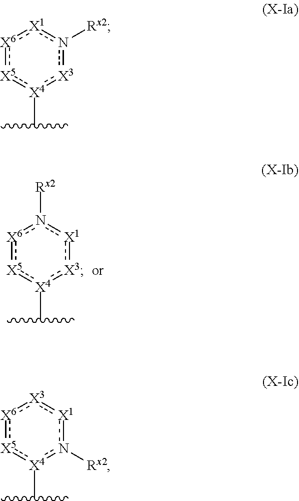
wherein:
X¹ is selected from the group consisting of: C(═O), C—OH, C═S, C—SH, C—NH₂, and C(═NH);
X³, X⁵, and X⁶ are each independently selected from the group consisting of: N, NH, N(R^{Xn}), CH, CR^{Xc}, C(═O), C(═S), C(—NH), and C(═NR^{Xn});
X⁴ is N or C;
R^{X2} is —H, R^{Xn}, or is absent when a double bond is present between NR^{X2} and an adjacent ring atom; and
each occurrence of [symbol] is independently a single bond or a double bond;
provided that formulas (X-Ia), (X-Ib), and (X-Ic) each include from 1-2 endocyclic double bonds;
provided that when X⁴ is C, then a double bond is present between X⁴ and an adjacent ring atom; and
provided that when formulas (X-Ia), (X-Ib), and (X-Ic) each include only 1 endocyclic double bond, then X⁴ is N and/or one or more of X³, X⁵, and X⁶ are each independently selected from the group consisting of: N, NH, N(R^{Xn}), C(═O), C(═S), C(═NH), and C(═NR^{Xn});
(B) pyridinyl, pyrmidinyl, pyrazinyl, pyridazinyl, or triazinyl each of which is optionally substituted with from 1-3 R^{Xc}, provided that any R^{Xc} group that is ortho or para to a ring nitrogen of (B) is other than —OH, —SH, or NH₂;
(C) a moiety having formula (X-II): 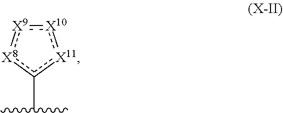
wherein:
X⁷ is C or N;
X⁸, X⁹, X¹⁰, and X¹¹ are each independently selected from the group consisting of: CH, C(R^{Xc}), N, N(H), N(R^{Xn}), O, S, C(═O), C(═S), C(═NH), and C(═NR^{Xn}); and
each is [symbol] independently a single bond or a double bond,
provided that from 1-4 of X⁷—X¹¹ is independently selected from group consisting of C, CH, C(R^{Xc}), C(═O), C(═S), C(═NH), and C(═NR^{Xn}), and (X-II) is aromatic;
(D) C₆₋₁₀ aryl optionally substituted with from 1-4 R^{Xc}; or
(E) bicyclic heteroaryl having 8-12 ring atoms, wherein from 1-5 ring atoms are heteroatoms each independently selected from the group consisting of: N, N(H), N(R^{Xn}), O, and S(═O)₀₋₂, and wherein one or more ring carbon atoms of the heteroaryl is optionally substituted with from 1-4 substituents each independently selected from the group consisting of oxo and R^{Xc};
each occurrence of R^{Xc} is independently selected from the group consisting of: R^{c}, R^{b}, and -(L^{b})_{b}-R^{b};
each occurrence of Ran is independently selected from the group consisting of: R^{d}, R^{b}, and -(L^{b})_{b}-R^{b};
R^{Y}, R^{4a}, R^{4b}, R^{5a}, and R^{5b} are each independently selected from the group consisting of:
—H, —OH, —SH, -halo, cyano, or azido;
C₁₋₆ alkyl, C₁₋₆ haloalkyl, C₂₋₆ alkenyl, C₂₋₆ haloalkenyl, C₂₋₆ alkynyl, or C₂₋₆ haloalkynyl, each of which is optionally substituted with from 1-6 R^{a};
C₁₋₄ alkoxy or C₁₋₄ thioalkoxy, each of which is optionally substituted with from 1-6 R^{a};
—OR⁹, —NR^{e}R^{f};
—R^{b} or -(L^{b})_{b}-R^{b};
—OP(═O)(OR′)(OR″); and
—OC(═O) (C₁₋₆ alkyl) optionally substituted with from 1-6 R^{a}; or
L¹, L², L³ and A are each independently selected from the group consisting of: —O—, —S—, —NR^{L1}—, and —C(R^{L2}) (R^{L2})—;
Y¹ and Y² are each independently selected from the group consisting of: O and S;
Y⁰ and Y³ are each independently selected from the group consisting of: —OH, —OR⁹, —SH, and —SR⁹,
R¹, R², R⁶, and R⁷ are each independently selected from the group consisting of: H, D, -halo, —OH, —SH, cyano, —OR⁹, —OC(═O)R′, —NR^{e}R^{f}, —NR^{e}C(═O)R′, —OP(—O)(OR′)(OR″), —OS(═O)₁₋₂R⁹, C₁₋₆ alkyl, C₁₋₆ haloalkyl, and —OR⁸;
R³ is selected from the group consisting of: H, D, -halo, —OH, —SH, cyano, —C(═O)OH, —C(═O)O(C₁₋₄ alkyl), —C(═O)NR′R″, —OR¹⁰, —OC(═O)R¹⁰, —NR^{e}R^{f}, —NR^{e}C(═O)R¹⁰, —OP(═O)(OR′)(OR″), —OS(═O)₁₋₂R¹⁰, C₁₋₆ alkyl, C₁₋₆ haloalkyl, and —OR⁸;
R^{3a} is selected from the group consisting of: —OH, —SH, —H, -halo, cyano, C₁₋₆ alkyl, C₁₋₆ haloalkyl, —C(═O)OH, —C(═O)O(C₁₋₄ alkyl), —C(═O)NR′R″, —OP(═O)(OR′)(OR″), C₁₋₄ alkoxy, C₁₋₄ haloalkoxy, —OR⁸, and —NR^{e}R^{f};
each occurrence of R⁸ is independently selected from the group consisting of:
—C(═O) C₁₋₂₀ alkyl optionally substituted with from 110 substituents independently selected from the group consisting of: R^{a}, R^{b}, and -(L^{b})_{b}-R^{b};
—C(═O)—(R^{b2})ₘ₁—R^{8b}, wherein each R^{b2} is independently a divalent R^{b} group, m1 is an integer from 1 to 6, and R^{8b} is —H or R^{e}; 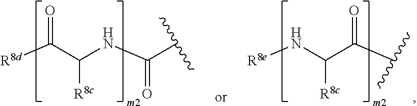
wherein:
m2 is an integer from 1 to 10;
each R^{8c} is independently selected from the group consisting of: —H; C₁₋₆ alkyl, which is optionally substituted with from 1-4 R^{a}; —R^{b}; and —(C₁₋₆ alkylene)-R^{b};
R^{8d} is selected from the group consisting of: —H, —OH, —C₁₋₄ alkoxy, and NR^{e}R^{f}; and
R^{8e} is selected from the group consisting of: —H, C₁₋₄ alkyl, C(═O) C₁₋₄ alkyl, and C(═O)OC₁₋₄ alkyl;
each occurrence of R⁹ is independently selected from the group consisting of: C₁₋₆ alkyl, 7-membered heterocyclyl, C₆₋₁₀ aryl, or 5- to 10-membered heteroaryl;
each occurrence of R¹⁰ is independently selected from the group consisting of: C₁₋₂₀ alkyl, C₁₋₂₀ haloalkyl, C₂₋₂₀ alkenyl, C₂₋₂₀ haloalkenyl, C₂₋₂₀ alkynyl, C₂₋₂₀ haloalkynyl, C₃₋₇ cycloalkyl, 3- to 7-membered heterocyclyl, C₆₋₁₀ aryl, or 5- to 10-membered heteroaryl;
each occurrence of R^{L1} is independently selected from the group consisting of: —H; C₁₋₆ alkyl, C₁₋₆ haloalkyl, C₂₋₆ alkenyl, C₂₋₆ haloalkenyl, C₂₋₆ alkynyl, or C₂₋₆ haloalkynyl, which is optionally substituted with 1-3 substituents each independently selected from the group consisting of NR′R″, —OH, C₁₋₄ alkoxy, and C₁₋₄ haloalkoxy; and —C(═O)R′;
each occurrence of R^{L2} is independently selected from the group consisting of: —H; -halo; —OH; —OR⁹; C₁₋₆ alkyl, C₁₋₆ haloalkyl, C₂₋₆ alkenyl, C₂₋₆ haloalkenyl, C₂₋₆ alkynyl, or C₂₋₆ haloalkynyl, which is optionally substituted with 1-3 substituents each independently selected from the group consisting of NR′R″, —OH, C₁₋₄ alkoxy, and C₁₋₄ haloalkoxy; and —C(═O)R′;
each occurrence of R^{a} is independently selected from the group consisting of: —H, —OH, -halo, —NR^{e}R^{f}, C₁₋₄ alkoxy, C₁₋₄ haloalkoxy, —C(═O)O(C₁₋₄ alkyl), —C(═O) (C₁₋₄ alkyl), —C(═O)OH, —C(═O)NR′R″, —S(═O)₁₋₂NR′R″, —S(═O)₁₋₂ (C₁₋₄ alkyl), and cyano;
each occurrence of R^{b} is independently selected from the group consisting of:
C₃₋₁₀ cycloalkyl or C₃₋₁₀ cycloalkenyl, each of which is optionally substituted with 1-4 R^{c};
heterocyclyl or heterocycloalkenyl of 3-10 ring atoms, wherein 1-3 ring atoms are heteroatoms, each independently selected from the group consisting of N, N(H), N(R^{d}), O, and S(═O)₀₋₂, and wherein the heterocyclyl or heterocycloalkenyl is optionally substituted with 1-4 R^{c};
heteroaryl of 5-10 ring atoms, wherein 1-3 ring atoms are heteroatoms, each independently selected from the group consisting of N, N(H), N(R^{d}), O, and S(═O)₀₋₂, and wherein the heteroaryl is optionally substituted with 1-4 R^{c}; and
C₆₋₁₀ aryl optionally substituted with 1-4 R^{c};
each occurrence of L^{b} is independently selected from the group consisting of: —O—, —NH—, —NR^{d}, —S(═O)₀₋₂, C(═O), and C₁₋₃ alkylene optionally substituted with 1-3 R^{a};
each occurrence of b is independently 1, 2, 3, or 4;
each occurrence of R^{e} is independently selected from the group consisting of: halo;
cyano; C₁₋₁₀ alkyl, which is optionally substituted with 1-6 independently selected R^{a}; C₂₋₆ alkenyl; C₂₋₆ alkynyl; C₁₋₄ alkoxy; C₁₋₄ haloalkoxy; —S(—O)₁₋₂ (C₁₋₄ alkyl); —NR^{e}R^{f}; —OH; —SH; —S(═O)₁₋₂NR′R″; —C₁₋₄ thioalkoxy; —NO₂; —OC(═O) (C₁₋₄ alkyl); —OC(═O) H; —C(═O) (C₁₋₄ alkyl); —C(═O) H; —C(═O)O(C₁₋₄ alkyl); —C(═O)OH; and —C(═O)NR′R″;
each occurrence of R^{d} is independently selected from the group consisting of: C₁₋₆ alkyl optionally substituted with 1-3 independently selected R^{a}; —C(═O) (C₁₋₄ alkyl); —C(═O)O(C₁₋₄ alkyl); —C(═O)NR′R″; —S(═O)₁₋₂NR′R″; —S(═O)₁₋₂ (C₁₋₄ alkyl); —OH; and C₁₋₄ alkoxy;
each occurrence of R^{e} and R^{f} is independently selected from the group consisting of: —H; C₁₋₆ alkyl or C₁₋₆ haloalkyl, which is optionally substituted with 1-3 substituents each independently selected from the group consisting of NR′R″, —OH, halo, C₁₋₄ alkoxy, and C₁₋₄ haloalkoxy; —C(═O)R′; —C(═O)OR′; —C(═O)NR′R″; C(═NR″)NR′R″; —C(═O) C(═O)R′; —S(═O)₁₋₂NR′R″; —S(═O)₁₋₂R′; —OH; and C₁₋₄ alkoxy; or
R^{e}, and R^{f} taken together with the N atom connecting them form a saturated or unsaturated 3- to 7-membered heterocyclyl; and
each occurrence of R′ and R″ is independently selected from the group consisting of: —H; C₁₋₄ alkyl or C₁₋₄ haloalkyl, which is optionally substituted with 1-3 substituents each independently selected from the group consisting of: halo, cyano, C₁₋₄ alkoxy, C₁₋₄ haloalkoxy, and —OH;
provided that at least one of the following is true:
a) R^{4a} is selected from the group consisting of: C₂₋₆ alkenyl, C₂₋₆ haloalkenyl, C₂₋₆ alkynyl, and C₂₋₆ haloalkynyl;
b) R^{4b} is NR^{e}R^{f}.

2.-27. (canceled)

28. The compound of Formula (X) according to claim 1, which is a compound of Formula (I-h), (I-h-1), (I-h-2), (I-h-3), (I-h-4) or (I-h-5): 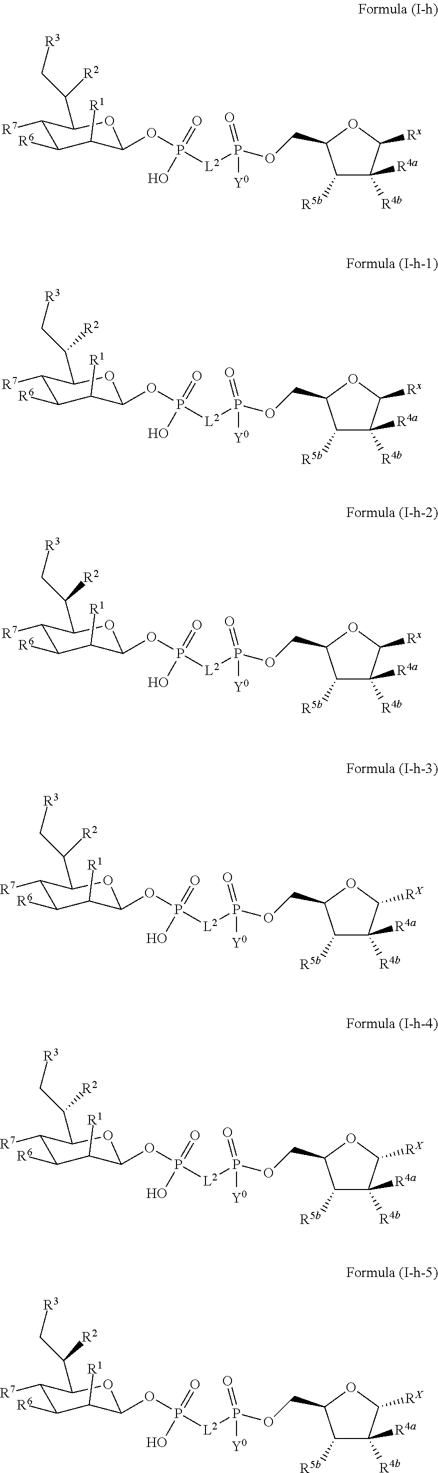
or a pharmaceutically acceptable salt, a stereoisomer, a stable isotope, a prodrug, or a tautomer thereof, wherein:
R^{X} is:
(A) a moiety having formula (X-Ia), (X-Ib), or (X-Ic): 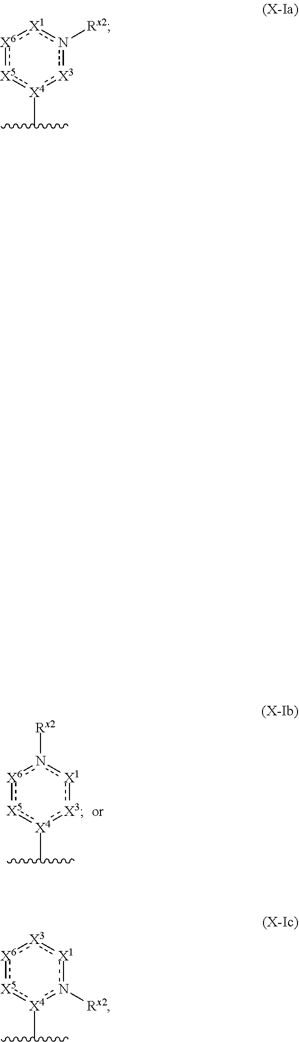
wherein:
is selected from the group consisting of: C(═O), C—OH, C—S, C—SH, C—NH₂, and C(═NH);
X³, X³, and X′ are each independently selected from the group consisting of: N, NH, N(R^{Xn}), CH, CR^{Xc}, C(═O), C(═S), C(═NH), and C(═NR^{Xn});
X⁴ is N or C;
R^{X2} is —H, R^{Xn}, or is absent when a double bond is present between NR^{X2} and an adjacent ring atom; and
each occurrence of [symbol] is independently a single bond or a double bond;
provided that formulas (X-Ia), (X-Ib), and (X-Ic) each include from 12 endocyclic double bonds;
provided that when X⁴ is C, then a double bond is present between X⁴ and an adjacent ring atom; and
provided that when formulas (X-Ia), (X-Ib), and (X-Ic) each include only 1 endocyclic double bond, then X⁴ is N and/or one or more of X³, X⁵, and X⁶ are each independently selected from the group consisting of: N, NH, N(Rn), C(═O), C(═S), C(═NH), and C(═NR^{Xn});
(B) pyridinyl, pyrmidinyl, pyrazinyl, pyridazinyl, or triazinyl each of which is optionally substituted with from 1-3 R^{Xc}, provided that any R^{Xc} group that is ortho or para to a ring nitrogen of (B) is other than —OH, —SH, or NH₂;
(C) a moiety having formula (X-II): 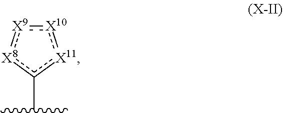
wherein:
X⁷ is C or N;
X⁸, X⁹, X¹⁰, and X¹¹ are each independently selected from the group consisting of: CH, C(R^{Xc}), N, N(H), N(R^{Xn}), O, S, C(═O), C(═S), C(═NH), and C(═NR^{Xn}); and
each [symbol] is independently a single bond or a double bond,
provided that from 1-4 of X⁷—X¹¹ is independently selected from group consisting of C, CH, C(R^{Xc}), C(═O), C(═S), C(═NH), and C(═NR^{Xn}), and (X-II) is aromatic;
(D) C₆₋₁₀ aryl optionally substituted with from 1-4 R^{Xc}, or
(E) bicyclic heteroaryl having 8-12 ring atoms, wherein from 1-5 ring atoms are heteroatoms each independently selected from the group consisting of: N, N(H), N(R^{Xn}), O, and S(═O)₀₋₂, and wherein one or more ring carbon atoms of the heteroaryl is optionally substituted with from 1-4 substituents each independently selected from the group consisting of oxo and R^{Xc};
each occurrence of R^{Xc} is independently selected from the group consisting of: R^{c}, R^{b}, and -(L^{b})_{b}-R^{b};
each occurrence of R^{Xn} is independently selected from the group consisting of: R^{d}, R^{b}, and -(L^{b})_{b}-R^{b};
R^{a} is selected from the group consisting of: C₂₋₆ alkenyl, C₂₋₆ haloalkenyl, C₂₋₆ alkynyl, and C₂₋₆ haloalkynyl;
R^{4b} and R^{5b} are each independently selected from the group consisting of:
—H, —OH, —SH, -halo, cyano, or azido;
C₁₋₆ alkyl, C₁₋₆ haloalkyl, C₂₋₆ alkenyl, C₂₋₆ haloalkenyl, C₂₋₆ alkynyl, or C₂₋₆ haloalkynyl, each of which is optionally substituted with from 1-6 R^{a};
C₁₋₄ alkoxy or C₁₋₄ thioalkoxy, each of which is optionally substituted with from 1-6 R^{a};
—OR⁹, —NR^{e}R^{f};
—R^{b} or -(L^{b})_{b}-R^{b};
—OP(═O)(OR′)(OR″); and
—OC(═O) (C₁₋₆ alkyl) optionally substituted with from 1-6 R^{a}; or
L² is selected from the group consisting of: —O—, —S—, —NR^{L1}—, and —C(R^{L2}) (R^{L2})—;
Y⁰ is selected from the group consisting of: —OH and —SH;
R³ is selected from the group consisting of: H, D, -halo, —OH, —SH, cyano, —OR¹⁰, —OC(═O)R¹⁰, —NR^{e}R^{f}, —NR^{e}C(═O)R¹⁰, —OP(═O)(OR′)(OR″), —OS(═O)₁₋₂R¹⁰, C₁₋₆ alkyl, and C₁₋₆ haloalkyl;
R¹, R², R⁶, and R⁷ are each independently selected from the group consisting of: H, D, -halo, —OH, —SH, cyano, —OR⁹, —OC(═O)R⁹, —NR^{e}R^{f}, —NR^{e}C(═O)R⁹, —OP(═O)(OR′)(OR″), —OS(═O)₁₋₂R⁹, C₁₋₆ alkyl, and C₁₋₆ haloalkyl;
each occurrence of R⁹ is independently selected from the group consisting of: C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl, 3- to 7-membered heterocyclyl, C₆₋₁₀ aryl, and 5- to 10-membered heteroaryl;
each occurrence of R¹⁰ is independently selected from the group consisting of: C₁₋₂₀ alkyl, C₁₋₂₀ haloalkyl, C₂₋₂₀ alkenyl, C₂₋₂₀ haloalkenyl, C₂₋₂₀ alkynyl, C₂₋₂₀ haloalkynyl, C₃₋₇ cycloalkyl, 3- to 7-membered heterocyclyl, C₆₋₁₀ aryl, and 5- to 10-membered heteroaryl;
each occurrence of R^{L1} is independently selected from the group consisting of: —H; C₁₋₆ alkyl, C₁₋₆ haloalkyl, C₂₋₆ alkenyl, C₂₋₆ haloalkenyl, C₂₋₆ alkynyl, or C₂₋₆ haloalkynyl, which is optionally substituted with 1-3 substituents each independently selected from the group consisting of NR′R″, —OH, C₁₋₄ alkoxy, and C₁₋₄ haloalkoxy; and —C(═O)R′;
each occurrence of R^{L2} is independently selected from the group consisting of: —H; -halo; —OH; —OR⁹; C₁₋₆ alkyl, C₁₋₆ haloalkyl, C₂₋₆ alkenyl, C₂₋₆ haloalkenyl, C₂₋₆ alkynyl, or C₂₋₆ haloalkynyl, which is optionally substituted with 1-3 substituents each independently selected from the group consisting of NR′R″, —OH, C₁₋₄ alkoxy, and C₁₋₄ haloalkoxy; and —C(═O)R′;
each occurrence of R^{a} is independently selected from the group consisting of: —H, —OH, -halo, —NR^{e}R^{f}, C₁₋₄ alkoxy, C₁₋₄ haloalkoxy, —C(═O)O(C₁₋₄ alkyl), —C(═O) (C₁₋₄ alkyl), —C(═O)OH, —C(═O)NR′R″, —S(═O)₁₋₂NR′R″, —S(═O)₁₋₂ (C₁₋₄ alkyl), and cyano;
each occurrence of R^{b} is independently selected from the group consisting of:
C₃₋₁₀ cycloalkyl or C₃₋₁₀ cycloalkenyl, each of which is optionally substituted with 1-4 R^{c};
heterocyclyl or heterocycloalkenyl of 3-10 ring atoms, wherein 1-3 ring atoms are heteroatoms, each independently selected from the group consisting of N, N(H), N(R^{d}), O, and S(═O)₀₋₂, and wherein the heterocyclyl or heterocycloalkenyl is optionally substituted with 1-4 R^{c};
heteroaryl of 5-10 ring atoms, wherein 1-3 ring atoms are heteroatoms, each independently selected from the group consisting of N, N(H), N(R^{d}), O, and S(═O)₀₋₂, and wherein the heteroaryl is optionally substituted with 1-4 R^{e}; and
C₆₋₁₀ aryl optionally substituted with 1-4 R^{c};
each occurrence of L^{b} is independently selected from the group consisting of: —O—, —NH—, —NR^{d}, —S(═O)₀₋₂, C(═O), and C₁₋₃ alkylene optionally substituted with 1-3 R^{a};
each occurrence of b is independently 1, 2, 3, or 4;
each occurrence of R^{e} is independently selected from the group consisting of: halo; cyano; C₁₋₁₀ alkyl, which is optionally substituted with 1-6 independently selected R^{a}; C₂₋₆ alkenyl; C₂₋₆ alkynyl; C₁₋₄ alkoxy; C₁₋₄ haloalkoxy; —S(—O)₁₋₂ (C₁₋₄ alkyl); —NR^{e}R^{f}; —OH; —SH; —S(═O)₁₋₂NR′R″; —C₁₋₄ thioalkoxy; —NO₂; —OC(═O) (C₁₋₄ alkyl); —OC(═O) H; —C(═O) (C₁₋₄ alkyl); —C(═O) H; —C(═O)O(C₁₋₄ alkyl); —C(═O)OH; and —C(═O)NR′R″;
each occurrence of R^{d} is independently selected from the group consisting of: C₁₋₆ alkyl optionally substituted with 1-3 independently selected R^{a}; —C(═O) (C₁₋₄ alkyl); —C(═O)O(C₁₋₄ alkyl); —C(═O)NR′R″; —S(═O)₁₋₂NR′R″; —S(═O)₁₋₂ (C₁₋₄ alkyl); —OH; and C₁₋₄ alkoxy;
each occurrence of R^{e} and R^{f} is independently selected from the group consisting of: —H; C₁₋₆ alkyl or C₁₋₆ haloalkyl, which is optionally substituted with 1-3 substituents each independently selected from the group consisting of NR′R″, —OH, halo, C₁₋₄ alkoxy, and C₁₋₄ haloalkoxy; —C(═O)R′; —C(═O)OR′; —C(═O)NR′R″; C(═NR″)NR′R″; —C(═O) C(═O)R′; —S(═O)₁₋₂NR′R″; —S(═O)₁₋₂R′; —OH; and C₁₋₄ alkoxy; and
each occurrence of R′ and R″ is independently selected from the group consisting of: —H; C₁₋₄ alkyl or C₁₋₄ haloalkyl, which is optionally substituted with 1-3 substituents each independently selected from the group consisting of: halo, cyano, C₁₋₄ alkoxy, C₁₋₄ haloalkoxy, and —OH.

29. The compound of claim 28, wherein:
R^{X} is selected from the group consisting of: 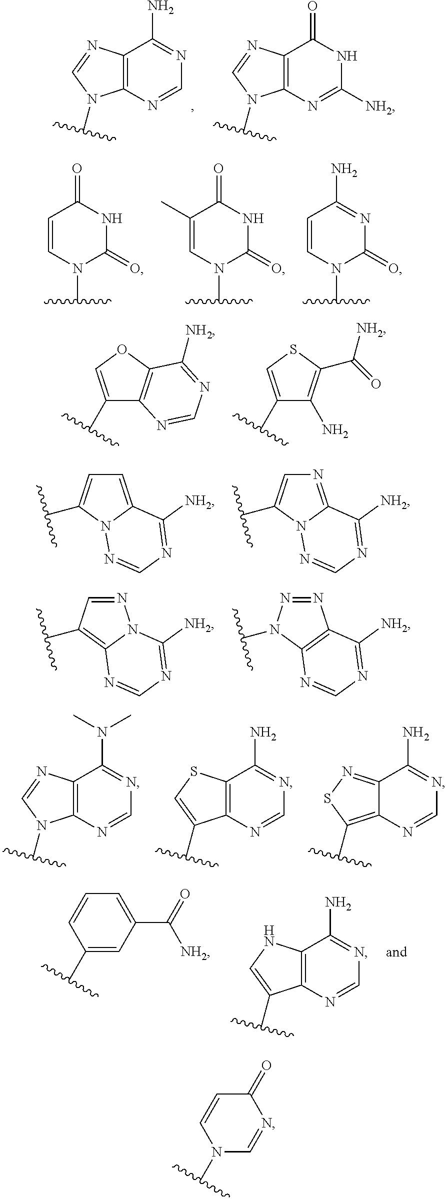
preferably, 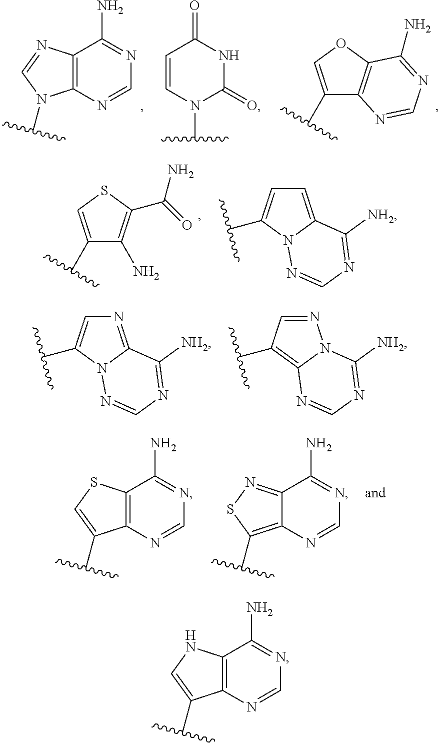
preferably, 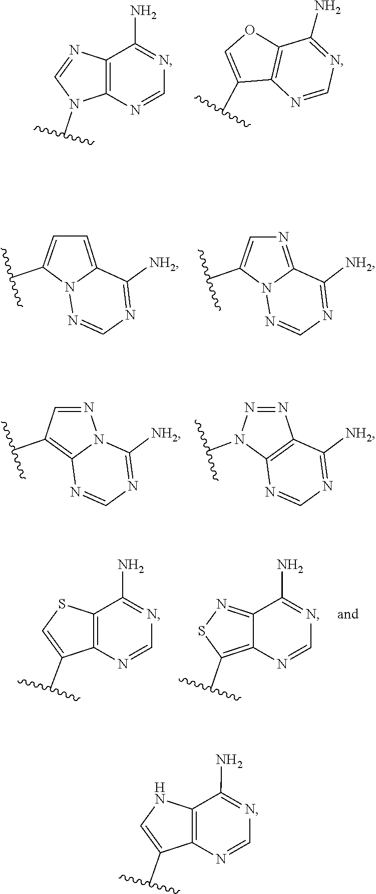
preferably, 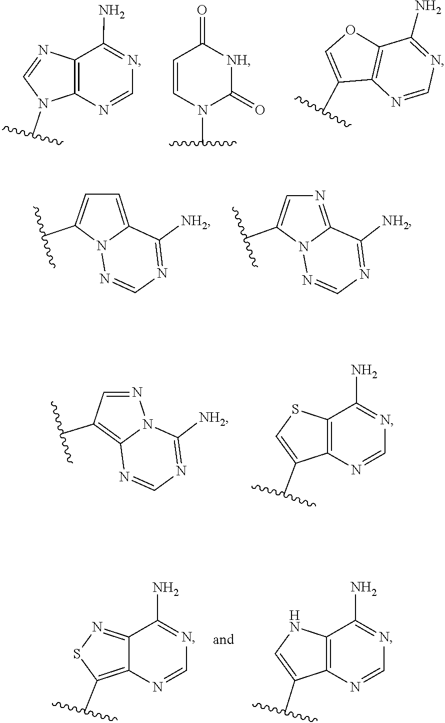
preferably, 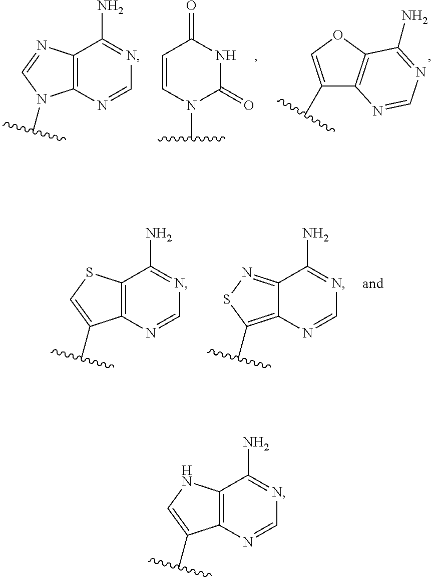
preferably, 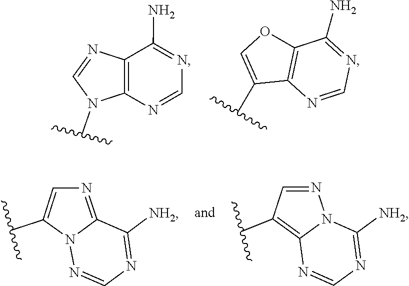 [SMILES: *n1cnc2c(N)ncnc21 ; *c1coc2c(N)ncnc12 ; *c1cnc2c(N)ncnn12 ; *c1cnn2c(N)ncnc12]
preferably, 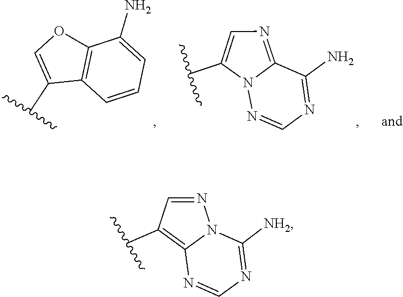
preferably, 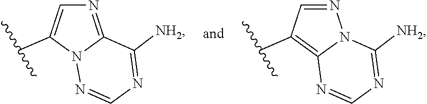 [SMILES: *c1cnc2c(N)ncnn12 ; *c1cnn2c(N)ncnc12]
preferably, 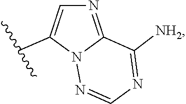 [SMILES: *c1cnc2c(N)ncnn12]
preferably, 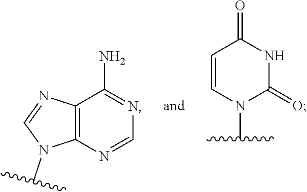 [SMILES: *n1cnc2c(N)ncnc21 ; *n1ccc(=O)[nH]c1=O]
R^{4a} is selected from the group consisting of: C₂₋₆ alkenyl, C₂₋₆ haloalkenyl, C₂₋₆ alkynyl, and C₂₋₆ haloalkynyl, preferably, C₂₋₆ alkenyl, C₂₋₆ haloalkenyl, C₂₋₆ alkynyl, and C₂₋₆ haloalkynyl that containing one to three double bond or triple bond, preferably C₂₋₆ alkenyl, and C₂₋₆ haloalkenyl that containing cumulated double bonds, preferably C₂₋₆ alkenyl, and C₂₋₆ haloalkenyl that containing conjugate double bonds, preferably C₂₋₆ alkenyl, and C₂₋₆ haloalkenyl that containing independent double bonds, preferably, ethenyl, propenyl, ethynyl, and propynyl, preferably, ethenyl, and ethynyl;
R^{4b} is selected from the group consisting of: —H, —OH, —OR⁹, —OC(═O)R⁹, —NR^{e}R^{f}, and -halo, preferably, —F, —OH, —OR⁹, and —NR^{e}R^{f}, preferably, —F, —OH, —OMe, and —NH₂, preferably, —F, —OH, and —OMe;
R^{5b} is independently selected from the group consisting of:
—H, —OH, —SH, -halo, cyano, or azido;
C₁₋₆ alkyl, C₁₋₆ haloalkyl, C₂₋₆ alkenyl, C₂₋₆ haloalkenyl, C₂₋₆ alkynyl, or C₂₋₆ haloalkynyl, each of which is optionally substituted with from 1-6 R^{a};
C₁₋₄ alkoxy or C₁₋₄ thioalkoxy, each of which is optionally substituted with from 1-6 R^{a};
—OR⁹, —NR^{e}R^{f};
—OC(═O) (C₁₋₆ alkyl) optionally substituted with from 1-6 R^{a};
preferably, R^{5b} is —OH;
L² is selected from the group consisting of: —O—, —S—, —NR^{L1}, and —C(R^{L2}) (R^{L2})—, preferably, —O—;
Y⁰ is selected from the group consisting of: —OH and —SH, preferably, —SH;
R³ is selected from the group consisting of: H, D, -halo, —OH, —SH, cyano, —OR¹⁰, —OC(═O)R¹⁰, —NR^{e}R^{f}, —NR^{e}C(═O)R¹⁰, —OP(═O)(OR′)(OR″), —OS(═O)₁₋₂R¹⁰, C₁₋₆ alkyl, and C₁₋₆ haloalkyl;
R¹, R², R⁶, and R⁷ are each independently selected from the group consisting of: H, D, -halo, —OH, —SH, cyano, —OR⁹, —OC(═O)R⁹, —NR^{e}R^{f}, —NR^{e}C(═O)R⁹, —OP(═O)(OR′)(OR″), —OS(═O)₁₋₂R⁹, C₁₋₆ alkyl, and C₁₋₆ haloalkyl;
preferably, R² is -halo, —OH, or —OC(═O)R⁹, preferably, —OH, or —OC(═O)R⁹, preferably, —OH, or —OC(═O) C₁₋₆ alkyl, preferably, —OH;
preferably, R³ is selected from the group consisting of: —OH, —OR¹⁰, and —OC(═O)R¹⁰, preferably, —OH, or —OC(═O) C₁₋₂₀ alkyl, preferably, —OH;
preferably, R¹, R⁶, and R⁷ are each independently —OH, or —OC(═O)R⁹, preferably, —OH, or —OC(═O) C₁₋₆ alkyl, preferably, —OH;
each occurrence of R⁹ is independently selected from the group consisting of: C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl, 3- to 7-membered heterocyclyl, C₆₋₁₀ aryl, or 5- to 10-membered heteroaryl;
each occurrence of R¹⁰ is independently selected from the group consisting of: C₁₋₂₀ alkyl, C₁₋₂₀ haloalkyl, C₂₋₂₀ alkenyl, C₂₋₂₀ haloalkenyl, C₂₋₂₀ alkynyl, C₂₋₂₀ haloalkynyl, C₃₋₇ cycloalkyl, 3- to 7-membered heterocyclyl, C₆₋₁₀ aryl, or 5- to 10-membered heteroaryl;
each occurrence of R^{L1} is independently selected from the group consisting of: —H; C₁₋₆ alkyl, C₁₋₆ haloalkyl, C₂₋₆ alkenyl, C₂₋₆ haloalkenyl, C₂₋₆ alkynyl, or C₂₋₆ haloalkynyl, which is optionally substituted with 1-3 substituents each independently selected from the group consisting of NR′R″, —OH, C₁₋₄ alkoxy, and C₁₋₄ haloalkoxy; and —C(═O)R′;
each occurrence of R^{L2} is independently selected from the group consisting of: —H; -halo; —OH; —OR⁹; C₁₋₆ alkyl, C₁₋₆ haloalkyl, C₂₋₆ alkenyl, C₂₋₆ haloalkenyl, C₂₋₆ alkynyl, or C₂₋₆ haloalkynyl, which is optionally substituted with 1-3 substituents each independently selected from the group consisting of NR′R″, —OH, C₁₋₄ alkoxy, and C₁₋₄ haloalkoxy; and —C(═O)R′;
each occurrence of R^{a} is independently selected from the group consisting of: —H; —OH; -halo; —NR^{e}R^{f}; C₁₋₄ alkoxy; C₁₋₄ haloalkoxy; —C(═O)O(C₁₋₄ alkyl); —C(═O) (C₁₋₄ alkyl); —C(═O)OH; —C(═O)NR′R″; —S(═O)₁₋₂NR′R″; —S(═O)₁₋₂ (C₁₋₄ alkyl); and cyano;
each occurrence of R^{e} and R^{f} is independently selected from the group consisting of: —H; C₁₋₆ alkyl or C₁₋₆ haloalkyl, which is optionally substituted with 1-3 substituents each independently selected from the group consisting of NR′R″, —OH, halo, C₁₋₄ alkoxy, and C₁₋₄ haloalkoxy; —C(═O)R′; —C(═O)OR′; —C(═O)NR′R″; C(═NR″)NR′R″; —C(═O) C(═O)R′; —S(═O)₁₋₂NR′R″; —S(═O)₁₋₂R′; —OH; and C₁₋₄ alkoxy; and
each occurrence of R′ and R″ is independently selected from the group consisting of: —H; C₁₋₄ alkyl or C₁₋₄ haloalkyl, which is optionally substituted with 1-3 substituents each independently selected from the group consisting of: halo, cyano, C₁₋₄ alkoxy, C₁₋₄ haloalkoxy, and —OH.

30. The compound of claim 29, wherein:
R^{X} is as defined in claim 29;
R^{4a} is selected from the group consisting of: C₂₋₆ alkenyl, C₂₋₆ haloalkenyl, C₂₋₆ alkynyl, and C₂₋₆ haloalkynyl, preferably, C₂₋₆ alkenyl, C₂₋₆ haloalkenyl, C₂₋₆ alkynyl, and C₂₋₆ haloalkynyl that containing one to three double bond or triple bond, preferably C₂₋₆ alkenyl, and C₂₋₆ haloalkenyl that containing cumulated double bonds, preferably C₂₋₆ alkenyl, and C₂₋₆ haloalkenyl that containing conjugate double bonds, preferably C₂₋₆ alkenyl, and C₂₋₆ haloalkenyl that containing independent double bonds, preferably, ethenyl, propenyl, ethynyl, and propynyl, preferably, ethenyl, and ethynyl;
R^{4b} is selected from the group consisting of: —H, —OH, —OR⁹, —OC(═O)R⁹, —NR^{e}R^{f}, and -halo, preferably, —F, —OH, —OR⁹, and —NR^{e}R^{f}, preferably, —F, —OH, —OMe, and —NH₂, preferably, —F, —OH, and —OMe;
R^{5b} is independently selected from the group consisting of:
—H, —OH, —SH, -halo, cyano, or azido;
C₁₋₆ alkyl, C₁₋₆ haloalkyl, C₂₋₆ alkenyl, C₂₋₆ haloalkenyl, C₂₋₆ alkynyl, or C₂₋₆ haloalkynyl, each of which is optionally substituted with from 1-6 R^{a};
—OR⁹, —NR^{e}R^{f};
preferably, R^{5b} is —OH;
L² is selected from the group consisting of: —O—, —S—, —NH—, —N(C₁₋₃ alkyl)-, —CH₂—, —CF₂—, —CHF—, —CH(C₁₋₃ alkyl)-, and —C(C₁₋₃ alkyl)OH—, preferably, —O—;
Y⁰ is selected from the group consisting of: —OH and —SH, preferably, —SH;
R² is selected from the group consisting of: -halo, —OH, —OR⁹, and —OC(═O)R′, preferably, -halo, —OH, or —OC(═O)R⁹, preferably, —OH, or —OC(═O)R⁹, preferably, —OH, or —OC(═O) C₁₋₆ alkyl, preferably, —OH;
R³ is selected from the group consisting of: —OH, —OR¹⁰, and —OC(═O)R¹⁰, preferably, —OH, or —OC(═O) C₁₋₂₀ alkyl, preferably, —OH;
R¹, R⁶, and R⁷ are each independently selected from the group consisting of: —OH, —OR⁹, and —OC(═O)R⁹, preferably, —OH, or —OC(═O) C₁₋₆ alkyl, preferably, —OH;
each occurrence of R⁹ is independently selected from the group consisting of: C₁₋₆ alkyl, and C₁₋₆ haloalkyl;
each occurrence of R¹⁰ is independently selected from the group consisting of: C₁₋₂₀ alkyl, C₁₋₂₀ haloalkyl, C₂₋₂₀ alkenyl, C₂₋₂₀ haloalkenyl, C₂₋₂₀ alkynyl, C₂₋₂₀ haloalkynyl, C₃₋₇ cycloalkyl, 3- to 7-membered heterocyclyl, C₆₋₁₀ aryl, and 5- to 10-membered heteroaryl;
each occurrence of R^{a} is independently selected from the group consisting of: —H; —OH; -halo; —NR^{e}R^{f}; C₁₋₄ alkoxy; C₁₋₄ haloalkoxy; —C(═O)O(C₁₋₄ alkyl); —C(═O) (C₁₋₄ alkyl); —C(═O)OH; —C(═O)NR′R″; —S(═O)₁₋₂NR′R″; —S(═O)₁₋₂ (C₁₋₄ alkyl); and cyano;
each occurrence of R^{e} and R^{f} is independently selected from the group consisting of: —H; C₁₋₆ alkyl or C₁₋₆ haloalkyl, which is optionally substituted with 1-3 substituents each independently selected from the group consisting of NR′R″, —OH, halo, C₁₋₄ alkoxy, and C₁₋₄ haloalkoxy; —C(═O)R′; —C(═O)OR′; —C(═O)NR′R″; C(═NR″)NR′R″; —C(═O) C(═O)R′; —S(═O)₁₋₂NR′R″; —S(═O)₁₋₂R′; —OH; and C₁₋₄ alkoxy; and
each occurrence of R′ and R″ is independently selected from the group consisting of: —H; C₁₋₄ alkyl or C₁₋₄ haloalkyl, which is optionally substituted with 1-3 substituents each independently selected from the group consisting of: halo, cyano, C₁₋₄ alkoxy, C₁₋₄ haloalkoxy, and —OH.

31. The compound of claim 29, wherein:
R^{X} is as defined in claim 29;
R^{4a} is selected from the group consisting of: C₂₋₆ alkenyl, C₂₋₆ haloalkenyl, C₂₋₆ alkynyl, and C₂₋₆ haloalkynyl, preferably, C₂₋₆ alkenyl, C₂₋₆ haloalkenyl, C₂₋₆ alkynyl, and C₂₋₆ haloalkynyl that containing one to three double bond or triple bond, preferably C₂₋₆ alkenyl, and C₂₋₆ haloalkenyl that containing cumulated double bonds, preferably C₂₋₆ alkenyl, and C₂₋₆ haloalkenyl that containing conjugate double bonds, preferably C₂₋₆ alkenyl, and C₂₋₆ haloalkenyl that containing independent double bonds, preferably, ethenyl, propenyl, ethynyl, and propynyl, preferably, ethenyl, and ethynyl;
R^{4b} is selected from the group consisting of: —OH, —OR⁹, —OC(═O)R⁹, —NR^{e}R^{f}, and -halo;
R^{5b} is selected from the group consisting of: —OH, —OR⁹, —NR^{e}R^{f}, and -halo;
L² is —O—;
Y⁰ is selected from the group consisting of: —OH and —SH;
R³ is selected from the group consisting of: H, D, -halo, —OH, —SH, cyano, —OR¹⁰, —OC(═O)R¹⁰, —NR^{e}R^{f}, —NR^{e}C(═O)R¹⁰, —OP(═O)(OR′)(OR″), —OS(═O)₁₋₂R¹⁰, C₁₋₆ alkyl, and C₁₋₆ haloalkyl;
R¹, R², R⁶, and R⁷ are each independently selected from the group consisting of: H, D, -halo, —OH, —SH, cyano, —OR⁹, —OC(═O)R⁹, —NR^{e}R^{f}, —NR^{e}C(═O)R⁹, —OP(—O)(OR′)(OR″), —OS(═O)₁₋₂R⁹, C₁₋₆ alkyl, and C₁₋₆ haloalkyl;
each occurrence of R⁹ is independently selected from the group consisting of: C₁₋₆ alkyl, 7-membered heterocyclyl, C₆₋₁₀ aryl, and 5- to 10-membered heteroaryl;
each occurrence of R¹⁰ is independently selected from the group consisting of: C₁₋₂₀ alkyl, C₁₋₂₀ haloalkyl, C₂₋₂₀ alkenyl, C₂₋₂₀ haloalkenyl, C₂₋₂₀ alkynyl, C₂₋₂₀ haloalkynyl, C₃₋₇ cycloalkyl, 3- to 7-membered heterocyclyl, C₆₋₁₀ aryl, and 5- to 10-membered heteroaryl;
each occurrence of R^{e} and R^{f} is independently selected from the group consisting of: —H; C₁₋₆ alkyl or C₁₋₆ haloalkyl, which is optionally substituted with 1-3 substituents each independently selected from the group consisting of NR′R″, —OH, halo, C₁₋₄ alkoxy, and C₁₋₄ haloalkoxy; and —C(═O)R^{b}; and
each occurrence of R′ and R″ is independently selected from the group consisting of: —H; C₁₋₄ alkyl or C₁₋₄ haloalkyl, which is optionally substituted with 1-3 substituents each independently selected from the group consisting of: halo, cyano, C₁₋₄ alkoxy, C₁₋₄ haloalkoxy, and —OH.

32. The compound of claim 29, wherein:
R^{X} is as defined in claim 29;
R^{4a} is selected from the group consisting of: C₂₋₆ alkenyl, C₂₋₆ haloalkenyl, C₂₋₆ alkynyl, and C₂₋₆ haloalkynyl, preferably, C₂₋₆ alkenyl, C₂₋₆ haloalkenyl, C₂₋₆ alkynyl, and C₂₋₆ haloalkynyl that containing one to three double bond or triple bond, preferably C₂₋₆ alkenyl, and C₂₋₆ haloalkenyl that containing cumulated double bonds, preferably C₂₋₆ alkenyl, and C₂₋₆ haloalkenyl that containing conjugate double bonds, preferably C₂₋₆ alkenyl, and C₂₋₆ haloalkenyl that containing independent double bonds, preferably, ethenyl, propenyl, ethynyl, and propynyl, preferably, ethenyl, and ethynyl;
R^{4b} is selected from the group consisting of: —OH, —OR⁹, —NR^{e}R^{f}, and -halo, preferably —OH, and -halo;
R^{5b} is selected from the group consisting of: —OH, —OR⁹, and —NR^{e}R^{f};
L² is —O—;
Y⁰ is selected from the group consisting of: —OH and —SH;
R² is selected from the group consisting of: -halo, —OH, —OR⁹, and —OC(═O)R⁹;
R³ is selected from the group consisting of: —OH, —OR¹⁰, and —OC(═O)R¹⁰;
R¹, R⁶, and R⁷ are each independently selected from the group consisting of: —OH, —OR⁹, and —OC(═O)R⁹;
each occurrence of R′ is independently selected from the group consisting of: C₁₋₆ alkyl, and C₁₋₆ haloalkyl;
each occurrence of R¹⁰ is independently selected from the group consisting of: C₁₋₂₀ alkyl, C₁₋₂₀ haloalkyl, C₂₋₂₀ alkenyl, C₂₋₂₀ haloalkenyl, C₂₋₂₀ alkynyl, C₂₋₂₀ haloalkynyl, C₃₋₇ cycloalkyl, 3- to 7-membered heterocyclyl, C₆₋₁₀ aryl, and 5- to 10-membered heteroaryl;
each occurrence of R^{e} and R^{f} is independently selected from the group consisting of: —H; C₁₋₆ alkyl or C₁₋₆ haloalkyl, which is optionally substituted with 1-3 substituents each independently selected from the group consisting of NR′R″, —OH, halo, C₁₋₄ alkoxy, and C₁₋₄ haloalkoxy; and —C(═O)R^{b}; and
each occurrence of R′ and R″ is independently selected from the group consisting of: —H; C₁₋₄ alkyl or C₁₋₄ haloalkyl, which is optionally substituted with 1-3 substituents each independently selected from the group consisting of: halo, cyano, C₁₋₄ alkoxy, C₁₋₄ haloalkoxy, and —OH.

33. The compound of claim 29, wherein:
R^{X} is as defined in claim 29;
R^{4a} is C₂₋₆ alkenyl, C₂₋₆ haloalkenyl, C₂₋₆ alkynyl, or C₂₋₆ haloalkynyl, preferably, C₂₋₆ alkenyl, C₂₋₆ haloalkenyl, C₂₋₆ alkynyl, or C₂₋₆ haloalkynyl that containing one to three double bond or triple bond, preferably C₂₋₆ alkenyl, or C₂₋₆ haloalkenyl that containing cumulated double bonds, preferably C₂₋₆ alkenyl, or C₂₋₆ haloalkenyl that containing conjugate double bonds, preferably C₂₋₆ alkenyl, or C₂₋₆ haloalkenyl that containing independent double bonds, preferably, ethenyl, propenyl, ethynyl, or propynyl, preferably, ethenyl, or ethynyl;
R^{4b} is selected from the group consisting of: —OH, —OMe, —NH₂, and —F, preferably, —F;
R^{5b} is selected from the group consisting of: —OH, —NH₂, —NHMe, —NMe₂, and —NHAc, preferably, —OH;
L² is —O—;
Y⁰ is selected from the group consisting of: —OH and —SH, preferably-SH;
R² is selected from the group consisting of: —F, —OH, and —OAc, preferably —OH, or —OAc, preferably, —OH;
R³ is selected from the group consisting of: —OH, and —OC(═O) C₁₋₂₀ alkyl, preferably —OH, and —OAc, preferably, —OH;
R¹, R⁶, and R⁷ are each independently selected from the group consisting of: —OH, and —OAc, preferably, —OH.

34. The compound of claim 28, wherein:
R^{X} is selected from the group consisting of: 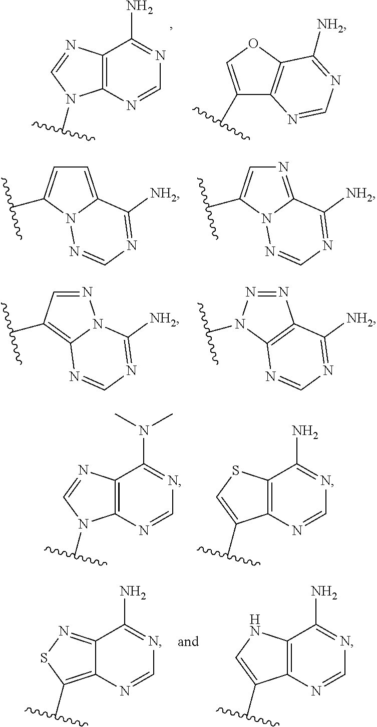
preferably, 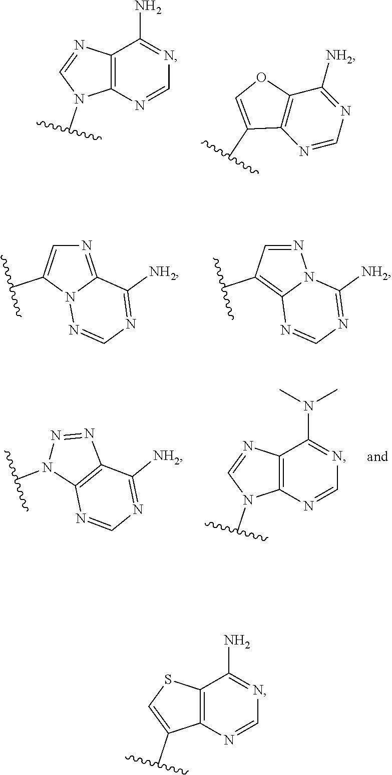
preferably, 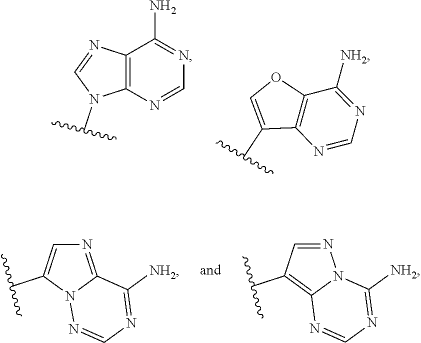 [SMILES: *n1cnc2c(N)ncnc21 ; *c1coc2c(N)ncnc12 ; *c1cnc2c(N)ncnn12 ; *c1cnn2c(N)ncnc12]
preferably, 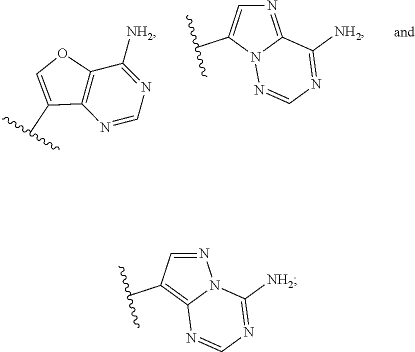 [SMILES: *c1coc2c(N)ncnc12 ; *c1cnc2c(N)ncnn12 ; *c1cnn2c(N)ncnc12]
R^{4a} is C₂₋₆ alkenyl, or C₂₋₆ haloalkenyl, preferably, ethenyl, propenyl, ethynyl, or propynyl, preferably, ethenyl, or ethynyl, preferably, ethenyl;
R^{4b} is selected from the group consisting of: H, —OH, —OMe, —NH₂, and —F, preferably, —OH, and —F, preferably, —OH;
R^{5b} is selected from the group consisting of: —OH, —NH₂, —NHMe, —NMe₂, and —NHAc, preferably, —OH;
L² is —O—;
Y⁰ is selected from the group consisting of: —OH and —SH, preferably-SH;
R² is selected from the group consisting of: —F, —OH, and —OC(═O) C₁₋₆ alkyl, preferably —OH, or —OAc, preferably, —OAc;
R³ is selected from the group consisting of: —OH, and —OC(═O) C₁₋₂₀ alkyl, preferably —OH, and —OAc, preferably, —OAc;
R¹, R⁶, and R⁷ are each independently selected from the group consisting of: —OH, and —OAc, preferably, —OAc.

35. The compound of Formula (X) according to claim 1, which is a compound of Formula (I-k), (I-k-1), (I-k-2), (I-k-3), (I-k-4) or (I-k-5): 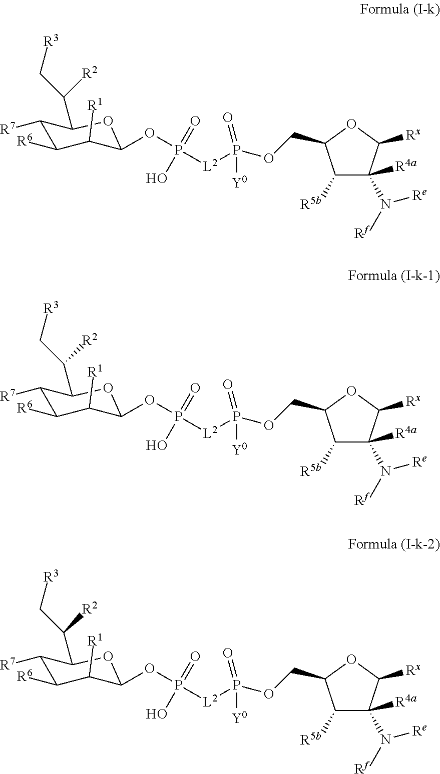 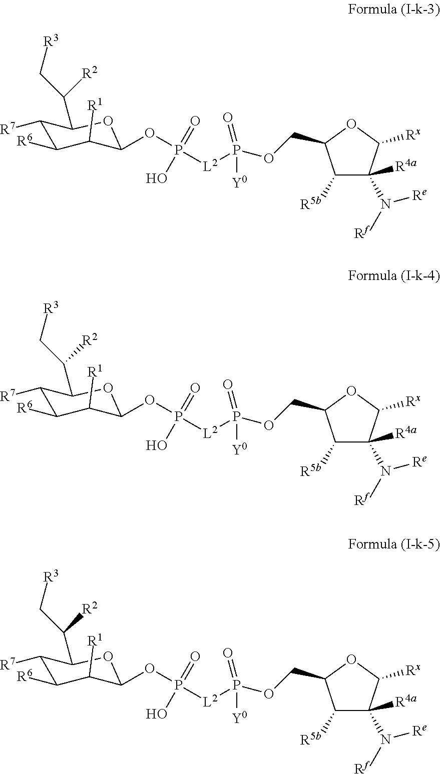
or a pharmaceutically acceptable salt, a stereoisomer, a stable isotope, a prodrug, or a tautomer thereof, wherein:
R^{X} is:
(A) a moiety having formula (X-Ia), (X-Ib), or (X-Ic): 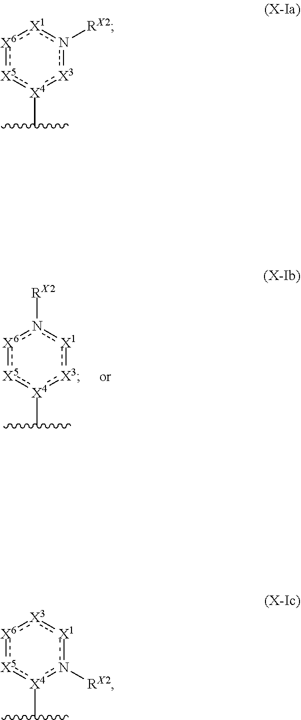
wherein:
X¹ is selected from the group consisting of: C(═O), C—OH, C═S, C—SH, C—NH₂, and C(═NH);
X³, X⁵, and X⁶ are each independently selected from the group consisting of: N, NH, N(R^{Xn}), CH, CR^{Xc}, C(═O), C(═S), C(═NH), and C(═NR^{Xn});
X⁴ is N or C;
R^{X2} is —H, R^{Xn}, or is absent when a double bond is present between NR^{X2} and an adjacent ring atom; and
each occurrence of [symbol] is independently a single bond or a double bond;
provided that formulas (X-Ia), (X-Ib), and (X-Ic) each include from 12 endocyclic double bonds;
provided that when X⁴ is C, then a double bond is present between X⁴ and an adjacent ring atom; and
provided that when formulas (X-Ia), (X-Ib), and (X-Ic) each include only 1 endocyclic double bond, then X⁴ is N and/or one or more of X³, X⁵, and X⁶ are each independently selected from the group consisting of: N, NH, N(R^{Xn}), C(═O), C(═S), C(═NH), and C(═NR^{Xn});
(B) pyridinyl, pyrmidinyl, pyrazinyl, pyridazinyl, or triazinyl each of which is optionally substituted with from 1-3 R^{Xc}, provided that any R^{Xc} group that is ortho or para to a ring nitrogen of (B) is other than —OH, —SH, or NH₂;
(C) a moiety having formula (X-II): 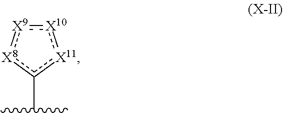
wherein:
X⁷ is C or N;
X⁸, X⁹, X¹⁰, and X¹¹ are each independently selected from the group consisting of: CH, C(R^{Xc}), N, N(H), N(R^{Xn}), O, S, C(═O), C(═S), C(═NH), and C(═NR^{Xn}); and
each [symbol] is independently a single bond or a double bond,
provided that from 1-4 of X⁷—X¹¹ is independently selected from group consisting of C, CH, C(R^{Xc}), C(═O), C(═S), C(═NH), and C(═NR^{Xn}), and (X-II) is aromatic;
(D) C₆₋₁₀ aryl optionally substituted with from 1-4 R^{Xc}; or
(E) bicyclic heteroaryl having 8-12 ring atoms, wherein from 1-5 ring atoms are heteroatoms each independently selected from the group consisting of: N, N(H), N(R^{Xn}), O, and S(═O)₀₋₂, and wherein one or more ring carbon atoms of the heteroaryl is optionally substituted with from 1-4 substituents each independently selected from the group consisting of oxo and R^{Xc};
each occurrence of R^{Xc} is independently selected from the group consisting of: R^{c}, R^{b}, and -(L^{b})_{b}-R^{b};
each occurrence of R^{Xn} is independently selected from the group consisting of: R^{d}, R^{b}, and -(L^{b})_{b}-R^{b};
R^{4a} and R^{5b} are independently selected from the group consisting of:
—H, —OH, —SH, -halo, cyano, or azido;
C₁₋₆ alkyl, C₁₋₆ haloalkyl, C₂₋₆ alkenyl, C₂₋₆ haloalkenyl, C₂₋₆ alkynyl, or C₂₋₆ haloalkynyl, each of which is optionally substituted with from 1-6 R^{a};
C₁₋₄ alkoxy or C₁₋₄ thioalkoxy, each of which is optionally substituted with from 1-6 R^{a};
—OR⁹, —NR^{e}R^{f};
—R^{b} or -(L^{b})_{b}-R^{b};
—OP(═O)(OR′)(OR″); and
—OC(═O) (C₁₋₆ alkyl) optionally substituted with from 1-6 R^{a};
L² is selected from the group consisting of: —O—, —S—, —NR^{L1}, and —C(R^{L2}) (R^{L2})—;
Y⁰ is selected from the group consisting of: —OH and —SH;
R³ is selected from the group consisting of: H, D, -halo, —OH, —SH, cyano, —OR¹⁰, —OC(═O)R¹⁰, —NR^{e}R^{f}, —NR^{e}C(═O)R¹⁰, —OP(═O)(OR′)(OR″), —OS(═O)₁₋₂R¹⁰, C₁₋₆ alkyl, and C₁₋₆ haloalkyl;
R¹, R², R⁶, and R⁷ are each independently selected from the group consisting of: H, D, -halo, —OH, —SH, cyano, —OR⁹, —OC(═O)R⁹, —NR^{e}R^{f}, —NR^{e}C(═O)R⁹, —OP(═O)(OR′)(OR″), —OS(═O)₁₋₂R⁹, C₁₋₆ alkyl, and C₁₋₆ haloalkyl;
each occurrence of R⁹ is independently selected from the group consisting of: C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl, 3- to 7-membered heterocyclyl, C₆₋₁₀ aryl, and 5- to 10-membered heteroaryl;
each occurrence of R¹⁰ is independently selected from the group consisting of: C₁₋₂₀ alkyl, C₁₋₂₀ haloalkyl, C₂₋₂₀ alkenyl, C₂₋₂₀ haloalkenyl, C₂₋₂₀ alkynyl, C₂₋₂₀ haloalkynyl, C₃₋₇ cycloalkyl, 3- to 7-membered heterocyclyl, C₆₋₁₀ aryl, and 5- to 10-membered heteroaryl;
each occurrence of R^{L1} is independently selected from the group consisting of: —H; C₁₋₆ alkyl, C₁₋₆ haloalkyl, C₂₋₆ alkenyl, C₂₋₆ haloalkenyl, C₂₋₆ alkynyl, or C₂₋₆ haloalkynyl, which is optionally substituted with 1-3 substituents each independently selected from the group consisting of NR′R″, —OH, C₁₋₄ alkoxy, and C₁₋₄ haloalkoxy; and —C(═O)R′;
each occurrence of R^{L2} is independently selected from the group consisting of: —H; -halo;
—OH; —OR⁹; C₁₋₆ alkyl, C₁₋₆ haloalkyl, C₂₋₆ alkenyl, C₂₋₆ haloalkenyl, C₂₋₆ alkynyl, or C₂₋₆ haloalkynyl, which is optionally substituted with 1-3 substituents each independently selected from the group consisting of NR′R″, —OH, C₁₋₄ alkoxy, and C₁₋₄ haloalkoxy; and —C(═O)R′;
each occurrence of R^{a} is independently selected from the group consisting of: —H; —OH; -halo; —NR^{e}R^{f}; C₁₋₄ alkoxy; C₁₋₄ haloalkoxy; —C(═O)O(C₁₋₄ alkyl); —C(═O) (C₁₋₄ alkyl); —C(═O)OH; —C(═O)NR′R″; —S(—O)₁₋₂NR′R″; —S(═O)₁₋₂ (C₁₋₄ alkyl); and cyano;
each occurrence of R^{b} is independently selected from the group consisting of:
C₃₋₁₀ cycloalkyl or C₃₋₁₀ cycloalkenyl, each of which is optionally substituted with 1-4 R^{c};
heterocyclyl or heterocycloalkenyl of 3-10 ring atoms, wherein 1-3 ring atoms are heteroatoms, each independently selected from the group consisting of N, N(H), N(R^{d}), O, and S(═O)₀₋₂, and wherein the heterocyclyl or heterocycloalkenyl is optionally substituted with 1-4 R^{c};
heteroaryl of 5-10 ring atoms, wherein 1-3 ring atoms are heteroatoms, each independently selected from the group consisting of N, N(H), N(R^{d}), O, and S(═O)₀₋₂, and wherein the heteroaryl is optionally substituted with 1-4 R^{c}; and
C₆₋₁₀ aryl optionally substituted with 1-4 R^{c};
each occurrence of L^{b} is independently selected from the group consisting of: —O—, —NH—, —NR^{d}, —S(═O)₀₋₂, C(═O), and C₁₋₃ alkylene optionally substituted with 1-3 R^{a};
each occurrence of b is independently 1, 2, 3, or 4;
each occurrence of R^{e} is independently selected from the group consisting of: halo; cyano; C₁₋₁₀ alkyl which is optionally substituted with 1-6 independently selected R^{a}; C₂₋₆ alkenyl; C₂₋₆ alkynyl; C₁₋₄ alkoxy; C₁₋₄ haloalkoxy; —S(═O)₁₋₂ (C₁₋₄ alkyl); —NR^{e}R^{f}; —OH; —SH; —S(═O)₁₋₂NR′R″; —C₁₋₄ thioalkoxy; —NO₂; —OC(═O) (C₁₋₄ alkyl); —OC(═O) H; —C(═O) (C₁₋₄ alkyl); —C(═O) H; —C(═O)O(C₁₋₄ alkyl); —C(═O)OH; and —C(═O)NR′R″;
each occurrence of R^{d} is independently selected from the group consisting of: C₁₋₆ alkyl optionally substituted with 1-3 independently selected R^{a}; —C(═O) (C₁₋₄ alkyl); —C(═O)O(C₁₋₄ alkyl); —C(═O)NR′R″; —S(═O)₁₋₂NR′R″; —S(═O)₁₋₂ (C₁₋₄ alkyl); —OH; and C₁₋₄ alkoxy;
each occurrence of R^{e} and R^{f} is independently selected from the group consisting of: —H;
C₁₋₆ alkyl or C₁₋₆ haloalkyl, which is optionally substituted with 1-3 substituents each independently selected from the group consisting of NR′R″, —OH, halo, C₁₋₄ alkoxy, and C₁₋₄ haloalkoxy; —C(═O)R′; —C(═O)OR′; —C(═O)NR′R″; C(═NR″)NR′R″; —C(═O) C(═O)R′; —S(═O)₁₋₂NR′R″; —S(═O)₁₋₂R′; —OH; and C₁₋₄ alkoxy; or
R^{e}, and R^{f} taken together with the N atom connecting them form a saturated or unsaturated 3- to 7-membered heterocyclyl; and
each occurrence of R′ and R″ is independently selected from the group consisting of: —H; C₁₋₄ alkyl or C₁₋₄ haloalkyl, which is optionally substituted with 1-3 substituents each independently selected from the group consisting of: halo, cyano, C₁₋₄ alkoxy, C₁₋₄ haloalkoxy, and —OH.

36. The compound of claim 35, wherein:
R^{X} is selected from the group consisting of: 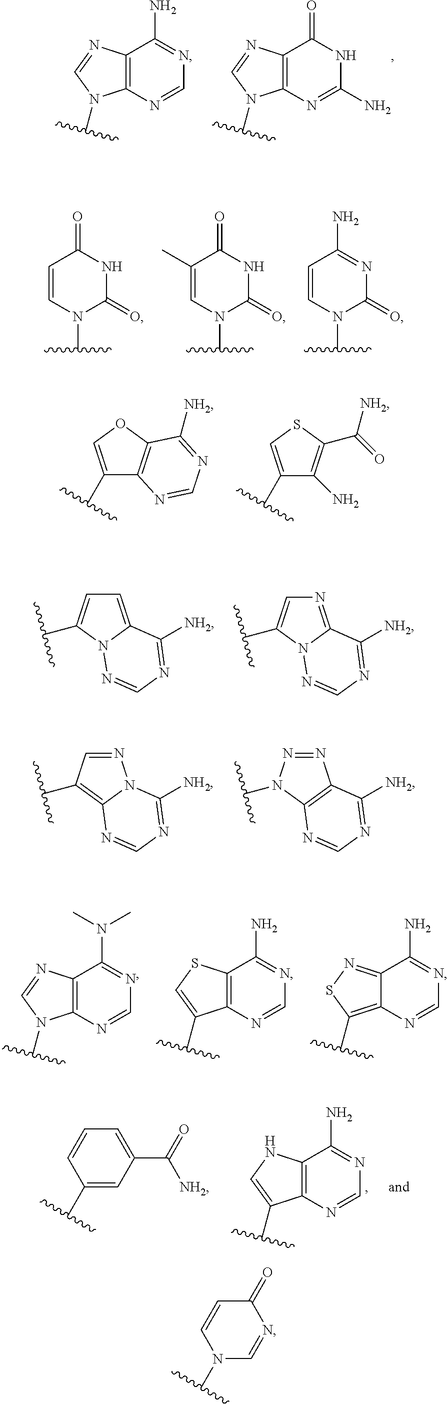
preferably, 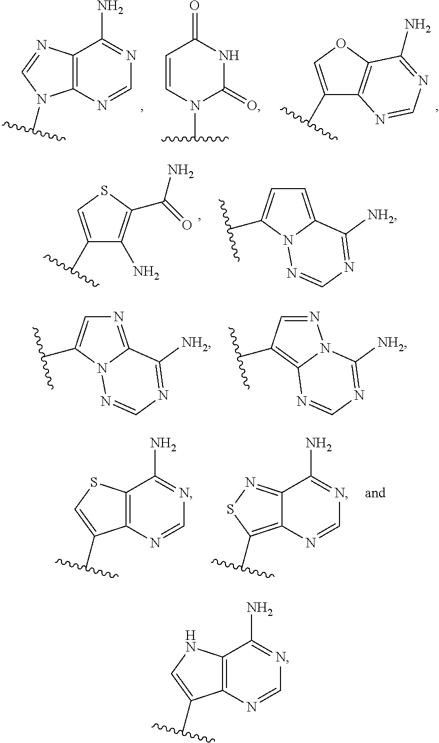
preferably, 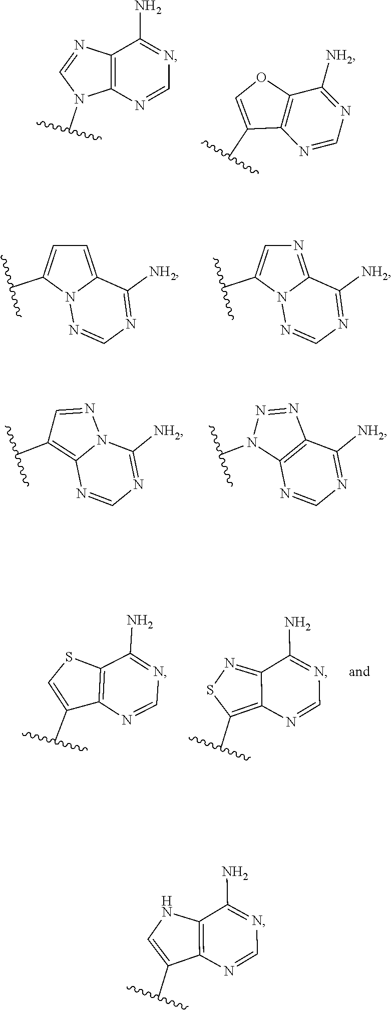
preferably, 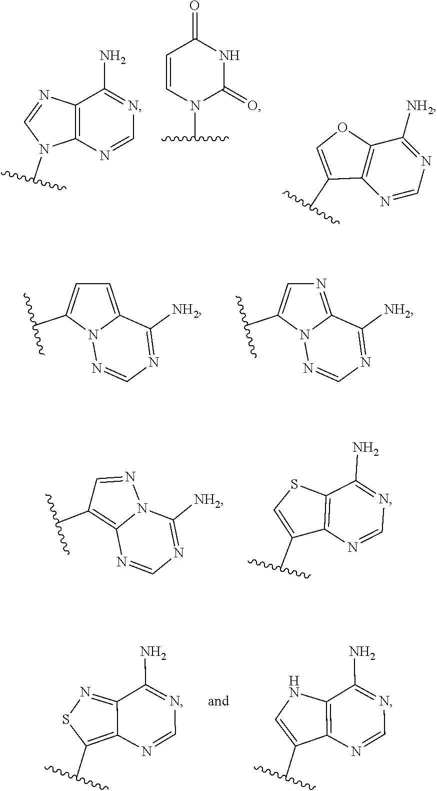
preferably, 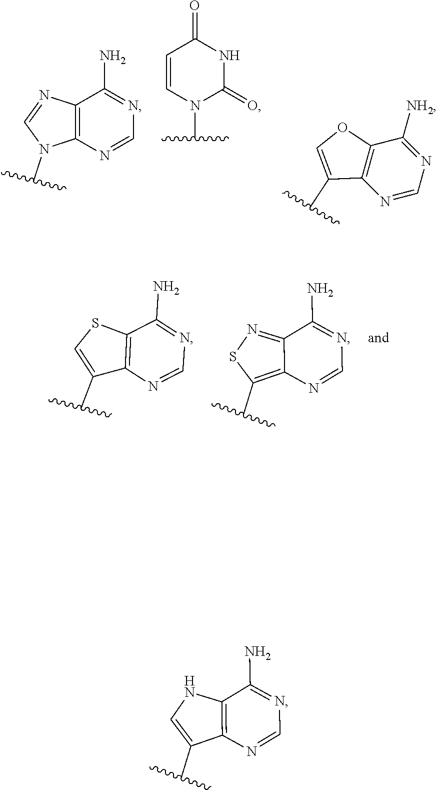
preferably, 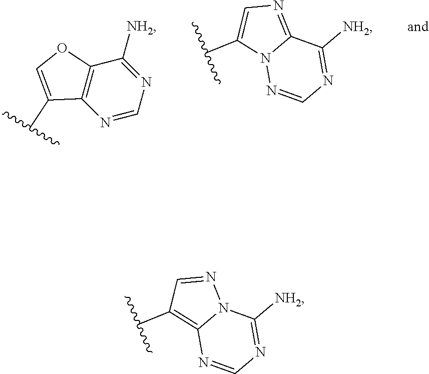 [SMILES: *c1coc2c(N)ncnc12 ; *c1cnc2c(N)ncnn12 ; *c1cnn2c(N)ncnc12]
preferably, 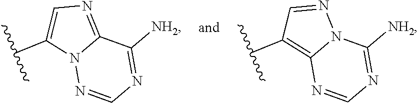 [SMILES: *c1cnc2c(N)ncnn12 ; *c1cnn2c(N)ncnc12]
preferably, 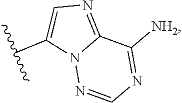 [SMILES: *c1cnc2c(N)ncnn12]
preferably, 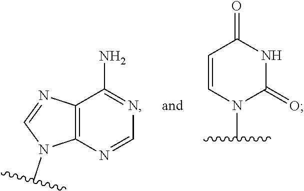 [SMILES: *n1cnc2c(N)ncnc21 ; *n1ccc(=O)[nH]c1=O]
R^{4a} and R^{5b} are independently selected from the group consisting of:
—H, —OH, —SH, -halo, cyano, or azido;
C₁₋₆ alkyl, C₁₋₆ haloalkyl, C₂₋₆ alkenyl, C₂₋₆ haloalkenyl, C₂₋₆ alkynyl, or C₂₋₆ haloalkynyl, each of which is optionally substituted with from 1-6 R^{a};
C₁₋₄ alkoxy or C₁₋₄ thioalkoxy, each of which is optionally substituted with from 1-6 R^{a};
—OR⁹, —NR^{e}R^{f};
—OC(═O) (C₁₋₆ alkyl) optionally substituted with from 1-6 R^{a};
preferably, R^{4a} is —H, and R^{5b} is —OH;
L² is selected from the group consisting of: —O—, —S—, —NR^{L1}—, and —C(R^{L2}) (R^{L2})—;
Y⁰ is selected from the group consisting of: —OH and —SH;
R³ is selected from the group consisting of: H, D, -halo, —OH, —SH, cyano, —OR¹⁰, —OC(═O)R¹⁰, —NR^{e}R^{f}, —NR^{e}C(═O)R¹⁰, —OP(═O)(OR′)(OR″), —OS(═O)₁₋₂R¹⁰, C₁₋₆ alkyl, and C₁₋₆ haloalkyl;
R¹, R², R⁶, and R⁷ are each independently selected from the group consisting of: H, D, -halo, —OH, —SH, cyano, —OR⁹, —OC(═O)R⁹, —NR^{e}R^{f}, —NR^{e}C(═O)R⁹, —OP(═O)(OR′)(OR″), —OS(═O)₁₋₂R⁹, C₁₋₆ alkyl, and C₁₋₆ haloalkyl;
each occurrence of R⁹ is independently selected from the group consisting of: C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl, 3- to 7-membered heterocyclyl, C₆₋₁₀ aryl, or 5- to 10-membered heteroaryl;
each occurrence of R¹⁰ is independently selected from the group consisting of: C₁₋₂₀ alkyl, C₁₋₂₀ haloalkyl, C₂₋₂₀ alkenyl, C₂₋₂₀ haloalkenyl, C₂₋₂₀ alkynyl, C₂₋₂₀ haloalkynyl, C₃₋₇ cycloalkyl, 3- to 7-membered heterocyclyl, C₆₋₁₀ aryl, or 5- to 10-membered heteroaryl;
each occurrence of R^{L1} is independently selected from the group consisting of: —H; C₁₋₆ alkyl, C₁₋₆ haloalkyl, C₂₋₆ alkenyl, C₂₋₆ haloalkenyl, C₂₋₆ alkynyl, or C₂₋₆ haloalkynyl, which is optionally substituted with 1-3 substituents each independently selected from the group consisting of NR′R″, —OH, C₁₋₄ alkoxy, and C₁₋₄ haloalkoxy; and —C(═O)R′;
each occurrence of R^{L2} is independently selected from the group consisting of: —H; -halo; —OH; —OR⁹; C₁₋₆ alkyl, C₁₋₆ haloalkyl, C₂₋₆ alkenyl, C₂₋₆ haloalkenyl, C₂₋₆ alkynyl, or C₂₋₆ haloalkynyl, which is optionally substituted with 1-3 substituents each independently selected from the group consisting of NR′R″, —OH, C₁₋₄ alkoxy, and C₁₋₄ haloalkoxy; and —C(═O)R′;
each occurrence of R^{a} is independently selected from the group consisting of: —H; —OH; -halo; —NR^{e}R^{f}; C₁₋₄ alkoxy; C₁₋₄ haloalkoxy; —C(═O)O(C₁₋₄ alkyl); —C(═O) (C₁₋₄ alkyl); —C(═O)OH; —C(═O)NR′R″; —S(═O)₁₋₂NR′R″; —S(═O)₁₋₂ (C₁₋₄ alkyl); and cyano;
each occurrence of R^{e} and R^{f} is independently selected from the group consisting of: —H; C₁₋₆ alkyl or C₁₋₆ haloalkyl, which is optionally substituted with 1-3 substituents each independently selected from the group consisting of NR′R″, —OH, halo, C₁₋₄ alkoxy, and C₁₋₄ haloalkoxy; —C(═O)R′; —C(═O)OR′; —C(═O)NR′R″; C(═NR″)NR′R″; —C(═O) C(═O)R′; —S(═O)₁₋₂NR′R″; —S(—O)₁₋₂R′; —OH; and C₁₋₄ alkoxy; or
R^{e}, and R^{f} taken together with the N atom connecting them form a saturated or unsaturated 3- to 7-membered heterocyclyl; and
each occurrence of R′ and R″ is independently selected from the group consisting of: —H; C₁₋₄ alkyl or C₁₋₄ haloalkyl, which is optionally substituted with 1-3 substituents each independently selected from the group consisting of: halo, cyano, C₁₋₄ alkoxy, C₁₋₄ haloalkoxy, and —OH;
preferably, both R^{e} and R^{f} are C₁₋₆ alkyl, such as -Me.

37. The compound of claim 36, wherein:
R^{X} is as defined in claim 36;
R^{a} is selected from the group consisting of: —H, -halo, C₁₋₆ alkyl, C₁₋₆ haloalkyl, C₂₋₆ alkenyl, C₂₋₆ haloalkenyl, C₂₋₆ alkynyl, and C₂₋₆ haloalkynyl;
R^{5b} is independently selected from the group consisting of:
—H, —OH, —SH, -halo, cyano, or azido;
C₁₋₆ alkyl, C₁₋₆ haloalkyl, C₂₋₆ alkenyl, C₂₋₆ haloalkenyl, C₂₋₆ alkynyl, or C₂₋₆ haloalkynyl, each of which is optionally substituted with from 1-6 R^{a};
—OR⁹, —NR^{e}R^{f};
preferably, R^{4a} is —H, and R^{5b} is —OH;
L² is selected from the group consisting of: —O—, —S—, —NH—, —N(C₁₋₃ alkyl)-, —CH₂—, —CF₂—, —CHF—, —CH(C₁₋₃ alkyl)-, and —C(C₁₋₃ alkyl)OH—;
Y⁰ is selected from the group consisting of: —OH and —SH;
R² is selected from the group consisting of: -halo, —OH, —OR⁹, and —OC(═O)R⁹;
R³ is selected from the group consisting of: —OH, —OR¹⁰, and —OC(═O)R¹⁰;
R¹, R⁶, and R⁷ are each independently selected from the group consisting of: —OH, —OR⁹, and —OC(═O)R⁹;
each occurrence of R′ is independently selected from the group consisting of: C₁₋₆ alkyl, and C₁₋₆ haloalkyl;
each occurrence of R¹⁰ is independently selected from the group consisting of: C₁₋₂₀ alkyl, C₁₋₂₀ haloalkyl, C₂₋₂₀ alkenyl, C₂₋₂₀ haloalkenyl, C₂₋₂₀ alkynyl, C₂₋₂₀ haloalkynyl, C₃₋₇ cycloalkyl, 3- to 7-membered heterocyclyl, C₆₋₁₀ aryl, and 5- to 10-membered heteroaryl;
each occurrence of R^{a} is independently selected from the group consisting of: —H; —OH; -halo; —NR^{e}R^{f}; C₁₋₄ alkoxy; C₁₋₄ haloalkoxy; —C(═O)O(C₁₋₄ alkyl); —C(═O) (C₁₋₄ alkyl); —C(═O)OH; —C(═O)NR′R″; —S(═O)₁₋₂NR′R″; —S(═O)₁₋₂ (C₁₋₄ alkyl); and cyano;
each occurrence of R^{e} and R^{f} is independently selected from the group consisting of: —H; C₁₋₆ alkyl or C₁₋₆ haloalkyl, which is optionally substituted with 1-3 substituents each independently selected from the group consisting of NR′R″, —OH, halo, C₁₋₄ alkoxy, and C₁₋₄ haloalkoxy; —C(═O)R′; —C(═O)OR′; —C(═O)NR′R″; C(═NR″)NR′R″; —C(═O) C(═O)R′; —S(═O)₁₋₂NR′R″; —S(═O)₁₋₂R′; —OH; and C₁₋₄ alkoxy; and
each occurrence of R′ and R″ is independently selected from the group consisting of: —H; C₁₋₄ alkyl or C₁₋₄ haloalkyl, which is optionally substituted with 1-3 substituents each independently selected from the group consisting of: halo, cyano, C₁₋₄ alkoxy, C₁₋₄ haloalkoxy, and —OH;
preferably, both R^{e} and R^{f} are C₁₋₆ alkyl, such as -Me.

38. The compound of claim 36, wherein:
R^{X} is as defined in claim 36;
R^{4a} is selected from the group consisting of: —H, -halo, C₁₋₆ alkyl, C₁₋₆ haloalkyl, C₂₋₆ alkenyl, C₂₋₆ haloalkenyl, C₂₋₆ alkynyl, and C₂₋₆ haloalkynyl;
R^{5b} is selected from the group consisting of: —OH, —OR⁹, —NR^{e}R^{f}, and -halo;
L² is —O—;
Y⁰ is selected from the group consisting of: —OH and —SH;
R³ is selected from the group consisting of: H, D, -halo, —OH, —SH, cyano, —OR¹⁰, —OC(═O)R¹⁰, —NR^{e}R^{f}, —NR^{e}C(═O)R¹⁰, —OP(═O)(OR′)(OR″), —OS(═O)₁₋₂R¹⁰, C₁₋₆ alkyl, and C₁₋₆ haloalkyl;
R¹, R², R⁶, and R⁷ are each independently selected from the group consisting of: H, D, -halo, —OH, —SH, cyano, —OR⁹, —OC(═O)R⁹, —NR^{e}R^{f}, —NR^{e}C(═O)R⁹, —OP(═O)(OR′)(OR″), —OS(═O)₁₋₂R⁹, C₁₋₆ alkyl, and C₁₋₆ haloalkyl;
each occurrence of R⁹ is independently selected from the group consisting of: C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl, 3- to 7-membered heterocyclyl, C₆₋₁₀ aryl, and 5- to 10-membered heteroaryl;
each occurrence of R¹⁰ is independently selected from the group consisting of: C₁₋₂₀ alkyl, C₁₋₂₀ haloalkyl, C₂₋₂₀ alkenyl, C₂₋₂₀ haloalkenyl, C₂₋₂₀ alkynyl, C₂₋₂₀ haloalkynyl, C₃₋₇ cycloalkyl, 3- to 7-membered heterocyclyl, C₆₋₁₀ aryl, and 5- to 10-membered heteroaryl;
each occurrence of R^{e} and R^{f} is independently selected from the group consisting of: —H; C₁₋₆ alkyl or C₁₋₆ haloalkyl, which is optionally substituted with 1-3 substituents each independently selected from the group consisting of NR′R″, —OH, halo, C₁₋₄ alkoxy, and C₁₋₄ haloalkoxy; and —C(═O)R^{b}; and
each occurrence of R′ and R″ is independently selected from the group consisting of: —H; C₁₋₄ alkyl or C₁₋₄ haloalkyl, which is optionally substituted with 1-3 substituents each independently selected from the group consisting of: halo, cyano, C₁₋₄ alkoxy, C₁₋₄ haloalkoxy, and —OH;
preferably, both R^{e} and R^{f} are C₁₋₆ alkyl, such as -Me.

39. The compound of claim 36, wherein:
R^{X} is as defined in claim 36;
R^{4a} is selected from the group consisting of: —H, -halo, C₁₋₆ alkyl, C₁₋₆ haloalkyl, C₂₋₆ alkenyl, C₂₋₆ haloalkenyl, C₂₋₆ alkynyl, and C₂₋₆ haloalkynyl;
R^{5b} is selected from the group consisting of: —OH, —OR⁹, and —NR^{e}R^{f};
L² is —O—;
Y⁰ is selected from the group consisting of: —OH and —SH;
R² is selected from the group consisting of: -halo, —OH, —OR⁹, and —OC(═O)R⁹;
R³ is selected from the group consisting of: —OH, —OR¹⁰, and —OC(═O)R¹⁰;
R¹, R⁶, and R⁷ are each independently selected from the group consisting of: —OH, —OR⁹, and —OC(═O)R⁹;
each occurrence of R′ is independently selected from the group consisting of: C₁₋₆ alkyl, and C₁₋₆ haloalkyl;
each occurrence of R¹⁰ is independently selected from the group consisting of: C₁₋₂₀ alkyl, C₁₋₂₀ haloalkyl, C₂₋₂₀ alkenyl, C₂₋₂₀ haloalkenyl, C₂₋₂₀ alkynyl, C₂₋₂₀ haloalkynyl, C₃₋₇ cycloalkyl, 3- to 7-membered heterocyclyl, C₆₋₁₀ aryl, and 5- to 10-membered heteroaryl;
each occurrence of R^{e} and R^{f} is independently selected from the group consisting of: —H; C₁₋₆ alkyl or C₁₋₆ haloalkyl, which is optionally substituted with 1-3 substituents each independently selected from the group consisting of NR′R″, —OH, halo, C₁₋₄ alkoxy, and C₁₋₄ haloalkoxy; and —C(═O)R^{b}; and
each occurrence of R′ and R″ is independently selected from the group consisting of: —H;
C₁₋₄ alkyl or C₁₋₄ haloalkyl, which is optionally substituted with 1-3 substituents each independently selected from the group consisting of: halo, cyano, C₁₋₄ alkoxy, C₁₋₄ haloalkoxy, and —OH;
preferably, both R^{e} and R^{f} are C₁₋₆ alkyl, such as -Me.

40. The compound of claim 36, wherein:
R^{X} is as defined in claim 36;
R^{4a} is —H, or Me, preferably-H;
R^{5b} is selected from the group consisting of: —OH, —NH₂, —NHMe, —NMe₂, and —NHAc, preferably —OH;
L² is —O—;
Y⁰ is selected from the group consisting of: —OH and —SH, preferably-SH;
R² is selected from the group consisting of: —F, —OH, and —OAc, preferably —OH;
R¹, R³, R⁶, and R⁷ are each independently selected from the group consisting of: —OH, and —OAc, preferably —OH;
each occurrence of R^{e} and R^{f} is-H; C₁₋₆ alkyl or —C(═O) C₁₋₄ alkyl, preferably-H or C₁₋₆ alkyl; preferably, both R^{e} and R^{f} are C₁₋₆ alkyl, such as -Me.

41. The compound of claim 35, wherein:
R^{X} is selected from the group consisting of: 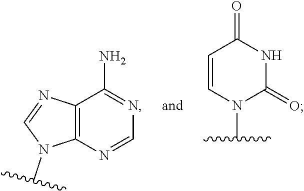 [SMILES: *n1cnc2c(N)ncnc21 ; *n1ccc(=O)[nH]c1=O]
preferably 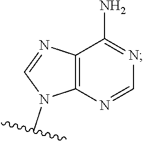 [SMILES: *n1cnc2c(N)ncnc21]
R^{4a} is —H, or Me, preferably-H;
R^{5b} is selected from the group consisting of: —OH, —NH₂, —NHMe, —NMe₂, and —NHAc, preferably —OH;
L² is —O—;
Y⁰ is selected from the group consisting of: —OH and —SH, preferably-SH;
R² is selected from the group consisting of: —F, —OH, and —OAc, preferably —OAc;
R³ is selected from the group consisting of: —OH, and —OC(═O) C₁₋₂₀ alkyl, preferably —OH, and —OAc, preferably, —OAc;
R¹, R⁶, and R⁷ are each independently selected from the group consisting of: —OH, and —OAc, preferably-OAc;
each occurrence of R^{e} and R^{f} is-H; C₁₋₆ alkyl or —C(═O) C₁₋₄ alkyl, preferably-H or C₁₋₆ alkyl; preferably, both R^{e} and R^{f} are C₁₋₆ alkyl, such as -Me.

42. A compound selected from the group consisting of the compounds delineated in Table 1, or a pharmaceutically acceptable salt, a stereoisomer, a stable isotope, a prodrug, or a tautomer thereof.

43. A pharmaceutical composition, comprising:
the compound, or the pharmaceutically acceptable salt, the stereoisomer, the stable isotope, the prodrug, or the tautomer thereof according to claim 1;
pharmaceutically acceptable excipient(s); and
optionally, one or more other therapeutic agents.

44. A kit, comprising:
a first container which contains the compound, or the pharmaceutically acceptable salt, the stereoisomer, the stable isotope, the prodrug, or the tautomer thereof according to claim 1; and
optionally, a second container which contains one or more other therapeutic agents; and
optionally, a third container which contains pharmaceutically acceptable excipient(s) for diluting or suspending the said compound and/or other therapeutic agent(s).

45.-46. (canceled)

47. A method of treating an immune and/or inflammatory related disease, or treating a cancer, or enhancing innate immunity in a subject in need thereof, or enhancing the efficacy of a vaccine, comprising administering to the subject a therapeutically effective amount of a compound, or a pharmaceutically acceptable salt, a stereoisomer, a stable isotope, a prodrug, or a tautomer thereof according to claim 1.

48. The method of claim 47, wherein the immune and/or inflammatory related disease is inflammatory bowel disease, ulcerative colitis, or Crohn's disease; wherein the cancer is selected from the group consisting of brain cancer, skin cancer, bladder cancer, ovarian cancer, breast cancer, gastric cancer, pancreatic cancer, hepatocellular cancer, prostate cancer, colorectal cancer, blood cancer, lung cancer, and bone cancer, or selected from the group consisting of: small cell lung cancer, non-small cell lung cancer, colorectal cancer, melanoma, renal cell carcinoma, head and neck cancer, Hodgkin's lymphoma, and bladder cancer; wherein the vaccine is a cancer vaccine, bacterial vaccine, a viral vaccine, or a parasite vaccine; or wherein the compound is an adjuvant; wherein for enhancing innate immunity, the therapeutically effective amount of a compound, or a pharmaceutically acceptable salt, a stereoisomer, a stable isotope, a prodrug, or a tautomer thereof is administered intramuscularly, intraperitoneally, intratumorally, or intravenously; or is administered comprising one or more immunotherapeutic agents; alternatively, wherein the one or more immunotherapeutic agents comprises a small molecule, an antibody, or a cytokine.

49.-67. (canceled)
